SEQUENCE LISTING (714 sequences)

SEQ ID NO: 1 - protein, 28 aa, synthetic construct
EIQALEEENAQLEQENAALEEEIAQLEY

SEQ ID NO: 2 - protein, 28 aa, synthetic construct
KIAQLKEKNAALKEKNQQLKEKIQALKY

SEQ ID NO: 3 - protein, 28 aa, synthetic construct
EIQQLEEEIAQLEQKNAALKEKNQALKY

SEQ ID NO: 4 - protein, 28 aa, synthetic construct
KIAQLKQKIQALKQENQQLEEENAALEY

SEQ ID NO: 5 - protein, 28 aa, synthetic construct
EIQQLEEEISQLEQKNSQLKEKNQQLKY

SEQ ID NO: 6 - protein, 28 aa, synthetic construct
KISQLKQKIQQLKQENQQLEEENSQLEY

SEQ ID NO: 7 - protein, 28 aa, synthetic construct
ENAALEEKIAQLKQKNAALKEEIQALEY

SEQ ID NO: 8 - protein, 28 aa, synthetic construct
KNAALKEEIQALEEENQALEEKIAQLKY

SEQ ID NO: 9 - protein, 28 aa, synthetic construct
EIQALEEKNAQLKQEIAALEEKNQALKY

SEQ ID NO: 10 - protein, 28 aa, synthetic construct
KIAQLKEENQQLEQKIQALKEENAALEY

SEQ ID NO: 11 - protein, 28 aa, synthetic construct
ENQALEQKNAQLKQEIAALEQEIAQLEY

SEQ ID NO: 12 - protein, 28 aa, synthetic construct
KNAQLKEENAALEEKIQQLKEKIQALKY

SEQ ID NO: 13 - protein, 28 aa, synthetic construct
ENQALEQEIAQLEQEIAALEQKNAQLKY

SEQ ID NO: 14 - protein, 28 aa, synthetic construct
KNAQLKEKIAALKEKIQQLKEENQALEY

SEQ ID NO: 15 - protein, 28 aa, synthetic construct
EIAALEAKIAALKAKNAALKAEIAALEA

SEQ ID NO: 16 - protein, 28 aa, synthetic construct
KIAALKAEIAALEAENAALEAKIAALKA

SEQ ID NO: 17 - protein, 28 aa, synthetic construct
ELAANEEELQQNEQKLAQIKQKLQAIKY

SEQ ID NO: 18 - protein, 22 aa, synthetic construct
LLPKNYHLENEVARLKKLVGER

SEQ ID NO: 19 - protein, 19 aa, synthetic construct
EELLSKNYHLENEVARLKK

SEQ ID NO: 20 - protein, 246 aa, synthetic construct
GPDIVMTQSPSSLSASVGDRVTITCRSSTGAVTTSNYASWVQEKPGKLFKGLIGGTNNRAPGVPSRFSGSLIGDKATLTISSLQPEDFATYFCALWYSNHWVFGQGTKVELKRGGGGSGGGGSGGGGSSGGGSEVKLLESGGGLVQPGGSLKLSCAVSGFSLTDYGVNWVRQAPGRGLEWIGVIWGDGITDYNSALKDRFIISKDNGKNTVYLQMSKVRSDDTALYYCVTGLFDYWGQGTLVTVSS

SEQ ID NO: 21 - protein, 117 aa, Porcine circovirus 2
MSPSKKNGRSGPQPHKRWVFTLNNPSEDERKKIRDLPISLFDYFIVGEEGNEEGRTPHLQGFANFVKKQTFNKVKWYLGARCHIEKAKGTDQQNKEYCSKEGNLLMECGAPRSQGQR

SEQ ID NO: 22 - protein, 120 aa, Duck circovirus
MAKSGNYSYKRWVFTINNPTFEDYVHVLEFCTLDNCKFAIVGEEKGANGTPHLQGFLNLRSNARAAALEESLGGRAWLSRARGSDEDNEEYCAKESTYLRVGEPVSKGRSSDLAEATSAV

SEQ ID NO: 23 - protein, 98 aa, unidentified
  source 1..98: note = Fava bean necrosis yellow virus
MARQVICWCFTLNNPLSPLSLHDSMKYLVYQTEQGEAGNIHFQGYIEMKKRTSLAGMKKLIPGAHFEKRRGTQGEARAYSMKEDTRLEGPWEYGEFVP

SEQ ID NO: 24 - protein, 130 aa, synthetic construct
MSEKKEIVKGRDWTFLVYPESAPENWRTILDETFMRWVESPLHDKDVNADGEIKKPHWHILLSSDGPITQTAVQKIIGPLNAPNAQKVGSAKGLVRYMVHLDNPEKYQYSLDEIVGHNGADVASYFELTA

SEQ ID NO: 25 - protein, 330 aa, synthetic construct
MMSIAQVRSAGSAGNYYTDKDNYYVLGSMGERWAGRGAEQLGLQGSVDKDVFTRLLEGRLPDGADLSRMQDGSNRHRPGYDLTFSAPKSVSMMAMLGGDKRLIDAHNQAVDFAVRQVEALASTRVMTDGQSETVLTGNLVMALFNHDTSRDQEPQLHTHAVVANVTQHNGEWKTLSSDKVGKTGFIENVYANQIAFGRLYREKLKEQVEALGYETEVVGKHGMWEMPGVPVEAFSGRSQTIREAVGEDASLKSRDVAALDTRKSKQHVDPEIKMAEWMQTLKETGFDIRAYRDAADQRADLRTLTPGPASQDGPDVQQAVTQAIAGLSER

SEQ ID NO: 26 - protein, 117 aa, synthetic construct
MSPSKKNGRSGPQPHKRWVFTLNNPSEDERKKIRDLPISLFDYFIVGEEGNEEGRTPHLQGFANFVKKQTFNKVKWYLGARCHIEKAKGTDQQNKEFCSKEGNLLMECGAPRSQGQR

SEQ ID NO: 27 - protein, 131 aa, synthetic construct
MAKEKARYFTFLLYPESIPSDWELKLETLGVPMAISPLHDKDKSSIKGQKYKKAHYHVLYIAKNPVTADSVRKKIKLLLGEKSLAMVQVVLNVENMYLYLTHESKDAIAKKKHVYDKADIKLINNFDIDRY

SEQ ID NO: 28 - protein, 286 aa, synthetic construct
MARYVVCWMFTINNPTTLPVMRDEIKYMVYQVERGQEGTRHVQGYVEMKRRSSLKQMRVFFPGAHLEKRKGSQEEARSYCMKEDTRIEGPFEFGAFKLSCNDNLFDVIQDMRETHKRPLEYLYDCPNTFDRSKDTLYRVQAELNKTRAMNSWRTSFSAWTSEVENIMAQPCHRRIIRVYGPNGGEGKTTYAKHLMKTKNAFYSPGGKSLDICRLYNYEDIVIFDIPRCKEDYLNYGLLEEFKNGIIQSGKYEPVLKVVEYVEVIVMANFLPKEGIFSEDRIKLVSC

SEQ ID NO: 29 - protein, 351 aa, synthetic construct
MASSSAPRFRVYSKYLFLTYPQCTLEPQYALDSLRTLLNKYEPLYIAAVRELHEDGSPHLHVLVQNKLRASITNPNALNLRMDTSPFSIFHPNIQAAKDCNQVRDYITKEVDSDANTAEWGTFVAVSTPGRKDRDADMKQIIESSSSREEFLSMVCNRFPFEWSIRLKDFEYTARHLFPDPVATYTPEFPTESLICHETIESWKNEHLYSESPGRHKSIYICGPTRTGKTSWARSLGTHNYYNSLVDFTTYDVNAKYNIIDDIPFKFTPNWKCFVGAQRDFTVNPKYGKRKVIRGGIPCIILVNPDEDWLKDMTPEQSDYMYSNAVVHYMYEGESFINYSFASGEDVTASQ

SEQ ID NO: 30 - protein, 357 aa, synthetic construct
MPRLFKIYAKNYFLTYPNCSLSKEEALSQLKNLETPTNKKYIKVCRELHENGEPHLHVLIQFEGKYQCKNQRFFDLVSPNRSAHFHPNIQAAKSSTDVKTYVEKDGDFIDFGVFQIDGRSARGGQQSANDAYAEALNSGNKSEALNILKEKAPKDYILQFHNLSSNLDRIFSPPLEVYVSPFLSSSFNQVPDELEEWVAENVVSSAARPWRPNSIVIEGDSRTGKTMWARSLGPHNYLCGHLDLSPKVYSNDAWYNVIDDVDPHYLKHFKEFMGAQRDWQSNTKYGKPIQIKGGIPTIFLCNPGPTSSYREYLDEEKNISLKNWALKNATFVTLYEPLFASINQGPTQDSQEETNKA

SEQ ID NO: 31 - protein, 334 aa, synthetic construct
MPSANKNFRFQSKYVFLTYPKCSSQRDDLFEFLWEKLTPFLIFFLGVATELHQDGTTHYHALIQLDKKPWIRDPSFFDFEGNHPNIQPARNSKQVLEYISKDGDIKTRGDFRDHKVSPRKSDARWRTIIQTATSKEEYLDMIKEEFPHEWATKLQWLEYSANKLFPPQPEAYVSPFTESDLRCHEDLAQWRDTHLYQEPRRTGARVPSLYICGPTRTGKTTWARSLGRHNYWNGTIDFTTYDEHATYNVIDDIPFKFVPLWKQLIGCQFDFTVNPKYGKKKKIKGGVPSIILTNRDEDWIPAMSEHQKEYFTDNCEIHYMDDGETFFARESSSH

SEQ ID NO: 32 - protein, 343 aa, Maize striate mosaic virus
MSHTSFRFRAKNVFLTYPRCPIGPEFLCDHLWNLVTPYDPLYVHVAQENHKDGGLHSHVLIQTRIEISTFDPTYFDYTGTSIPGAVVFHPNIQACRNVRDCLAYIRKNTINEVSKGAFKCSGAGRPKKQDSAPSRDAKMCQIMSSATSRSDYLSMVRGAFPFEWATKLAQFEYSASKLFPDAPTTNAIPSNIDLTCHENIMPWLRDDLYTERGQGRRPRSLYICGRTRTGKTTWARSLGPHNYFQHQVDYFAWSEEATYVVIDDIPFKFCPNWKAIVGSQHDYTVNPKYGKKRKLKGGIPAIILVNEDEDWLMSMTTSQIDWFLENCVVYHMTPGESFFNYAE

SEQ ID NO: 33 - protein, 349 aa, synthetic construct
MPRNPKSFRLAARNIFLTYPQCDIPKDEALQMLQTLSWSVVKPTYIRVAREEHSDGSPHLHCLIQLSGKSNIKDARLFDITHPRRSANFHPNIQAAKDTNAVKNYITKDGDYCESGQYKVSGGTKANKDDVYHNAVNAGCVEEALAIIKAGDPKTFIVSYHNVRANIERLFTKAPEPWAPPFQLSSFTNVPDEMRSWADDYFGRSAAARPERPISIIVEGDSRTGKTMWARALGPHNYLSGHLDFNSKVFSNNVEYNVIDDIAPHYLKLKHWKELIGAQRDWQSNCKYGRPVQIKGGIPSIVLCNPGEGSSYISFLNKEENASLRAWTIKNAKFITLEAPLYQSTAQDC

SEQ ID NO: 34 - protein, 352 aa, Tomato golden mosaic virus
MPSHPKRFQINAKNYFLTYPQCSLSKEESLSQLQALNTPINKKFIKICRELHEDGQPHLHVLIQFEGKYCCQNQRFFDLVSPTRSAHFHPNIQRAKSSSDVKTYIDKDGDTLVWGEFQVDGRSARGGCQTSNDAAAEALNASSKEEALQIIREKIPEKYLFQFHNLNSNLDRIFDKTPEPWLPPFHVSSFTNVPDEMRQWAENYFGKSSAARPERPISIIIEGDSRTGKTMWARSLGPHNYLSGHLDLNSRVYSNKVEYNVIDDVTPQYLKLKHWKELIGAQRDWQTNCKYGKPVQIKGGIPSIVLCNPGEGASYKVFLDKEENTPLKNWTFHNAKFVFLNSPLYQSSTQSS

SEQ ID NO: 35 - protein, 298 aa, synthetic construct
MLSHMVLTRQDIGRAASYYEDGADDYYAKDGDASEWQGKGAEELGLSGEVDSKRFRELLAGNIGEGHRIMRSATRQDSKERIGLDLTFSAPKSVSLQALVAGDAEIIKAHDRAVARTLEQAEARAQARQKIQGKTRIETTGNLVIGKFRHETSRERDPQLHTHAVILNMTKRSDGQWRALKNDEIVKRTRYLGAVYNAELAHELQKLGYQLRYGKDGNFDLAHIDRQQIEGFSKRTEQIAEWYAARGLDPNSVSLEQKQAAKVLSRAKKTSVDREALRAEWQATAKELGIDFSRREWS

SEQ ID NO: 36 - protein, 220 aa, synthetic construct
MAMYHFQNKFVSKANGQSATAKSAYNSASRIKDFKENEFKDYSNKQCDYSEILLPNNADDKFKDREYLWNKVHDVENRKNSQVAREIIIGLPNEFDPNSNIELAKEFAESLSNEGMIVDLNIHKINEENPHAHLLCTLRGLDKNNEFEPKRKGNDYIRDWNTKEKHNEWRKRWENVQNKHLEKNGFSVRVSADSYKNQNIDLEPTKKEGWKARKFEDETG

SEQ ID NO: 37 - protein, 186 aa, synthetic construct
MAIYHLTAKTGSRSGGQSARAKADYIQREGKYARDMDEVLHAESGHMPEFVERPADYWDAADLYERANGRLFKEVEFALPVELTLDQQKALASEFAQHLTGAERLPYTLAIHAGGGENPHCHLMISERINDGIERPAAQWFKRYNGKTPEKGGAQKTEALKPKAWLEQTREAWADHANRALERAGH

SEQ ID NO: 38 - protein, 271 aa, synthetic construct
MHLFTDVVSVDIMTTMQRTHTNAELIRWFKIFRDLDIHKWVIGLEEGKGGYGHWQVRCNVRVEVVNDWTAYLRAVFGWLGPISIWTEECSDKYTYETKEGKYWASWDTMGARQQRFGKMRWNQEGAVQALQRTNDREIVVWYDEQGNMGKSWLCGHLFETGQAYYIPPYMTSIQSMIQTVASLVLQDRDSGYPPRPLIVIDIPRSWKWSTELYTAIEAIKDGLIMDPRYGARPVNIHGIKVIVLTNTKPKLDKLSEDRWVLYDPMDYLMML

SEQ ID NO: 39 - protein, 271 aa, synthetic construct
MHLFTEGISVDIMTTMQRTHTNAELIRWFKIFRDLDIHKWVIGLEEGKGEYGHWQVRCNVRVEVVNDWTAYLRAVFGWLGPISIWTEECSDKYTYETKEGKYWASWDTMGARQQRFGKMRWNQEGAVQALQRTNDREIVVWYDEGGNMGKSWLCGHLFETGQAYYIPPYMTSIQSMIQTVASLVLQDRETGYPPRPLIVIDIPRSWKWSTELYTAIEAIKGGLIMDPRYGARPVNIHGTKVIVLTNTKPKLDKLSEDRWVLYDPMDYLMML

SEQ ID NO: 40 - protein, 163 aa, synthetic construct
MGARQQRFGKMRWNQEGALQALQRTNDREIVVWYDQDGNMGKSWLCGHLYETGQAYYIPPYMTSIQSMIQTVASLVLQDRESGYPPRPLIVIDIPRSRKWSTELYTAIEAIKDGLIMDPRYGARPVNIHGTKVIVLTNTKPKLDKLSEDRWVLYDPLLYMMGH

SEQ ID NO: 41 - DNA, 17 bp, Porcine circovirus 2
  source 1..17: mol_type = other DNA
ctgtaagtattaccagc

SEQ ID NO: 42 - DNA, 17 bp, Duck circovirus
  source 1..17: mol_type = other DNA
gttatattattaccggc

SEQ ID NO: 43 - DNA, 17 bp, unidentified
  source 1..17: mol_type = other DNA; note = Fava bean necrosis yellow virus
ggcttagtattaccccc

SEQ ID NO: 44 - DNA, 23 bp, synthetic construct
  source 1..23: mol_type = other DNA
tgcttccgtactacgacccccca

SEQ ID NO: 45 - DNA, 20 bp, synthetic construct
  source 1..20: mol_type = other DNA
tttgcgtggggtgtggtgct

SEQ ID NO: 46 - DNA, 17 bp, synthetic construct
  source 1..17: mol_type = other DNA
ggcttattattaccccc

SEQ ID NO: 47 - DNA, 17 bp, synthetic construct
  source 1..17: mol_type = other DNA
gttataatattaccccc

SEQ ID NO: 48 - DNA, 17 bp, synthetic construct
  source 1..17: mol_type = other DNA
cgtataatattaccgga

SEQ ID NO: 49 - DNA, 17 bp, synthetic construct
  source 1..17: mol_type = other DNA
aaattaatattaccggc

SEQ ID NO: 50 - DNA, 17 bp, Maize striate mosaic virus
  source 1..17: mol_type = other DNA
ataataatattacgcgc

SEQ ID NO: 51 - DNA, 17 bp, synthetic construct
  source 1..17: mol_type = other DNA
gcaataatattaccgga

SEQ ID NO: 52 - DNA, 17 bp, Tomato golden mosaic virus
  source 1..17: mol_type = other DNA
cgtttaatattaccgga

SEQ ID NO: 53 - DNA, 27 bp, synthetic construct
  source 1..27: mol_type = other DNA
gcgcaccgaaaggtgcgtattgtctat

SEQ ID NO: 54 - DNA, 45 bp, synthetic construct
  source 1..45: mol_type = other DNA
acgcgaacggaacgttcgcataagtgcgcccttacgggatttaac

SEQ ID NO: 55 - DNA, 38 bp, synthetic construct
  source 1..38: mol_type = other DNA
ccagtttctcgaagagaaaccggtaagtgcaccctccc

SEQ ID NO: 56 - DNA, 14 bp, synthetic construct
  source 1..14: mol_type = other DNA
aatagttacgaaaa

SEQ ID NO: 57 - protein, 182 aa, synthetic construct
MDKDCEMKRTTLDSPLGKLELSGCEQGLHRIIFLGKGTSAADAVEVPAPAAVLGGPEPLMQATAWLNAYFHQPEAIEEFPVPALHHPVFQQESFTRQVLWKLLKVVKFGEVISYSHLAALAGNPAATAAVKTALSGNPVPILIPCHRVVQGDLDVGGYEGGLAVKEWLLAHEGHRLGKPGLG

SEQ ID NO: 58 - protein, 182 aa, synthetic construct
MDKDCEMKRTTLDSPLGKLELSGCEQGLHRIIFLGKGTSAADAVEVPAPAAVLGGPEPLIQATAWLNAYFHQPEAIEEFPVPALHHPVFQQESFTRQVLWKLLKVVKFGEVISESHLAALVGNPAATAAVNTALDGNPVPILIPCHRVVQGDSDVGPYLGGLAVKEWLLAHEGHRLGKPGLG

SEQ ID NO: 59 - protein, 297 aa, synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 60 - protein, 120 aa, synthetic construct
MAEAGITGTWYNQHGSTFTVTAGADGNLTGQYENRAQGTGCQNSPYTLTGRYNGTKLEWRVEWNNSTENCHSRTEWRGQYQGGAEARINTQWNLTYEGGSGPATEQGQDTFTKVKPSAAS

SEQ ID NO: 61 - protein, 129 aa, synthetic construct
MFDASNFKDFSSIASASSSWQNQSGSTMIIQVDSFGNVSGQYVNRAQGTGCQNSPYPLTGRVNGTFIAFSVGWNNSTENCNSATGWTGYAQVNGNNTEIVTSWNLAYEGGSGPAIEQGQDTFQYVPTTE

SEQ ID NO: 62 - protein, 117 aa, synthetic construct
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIY

SEQ ID NO: 63 - protein, 130 aa, synthetic construct
MASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIY

SEQ ID NO: 64 - protein, 128 aa, synthetic construct
MSKTIVLSVGEATRTLTEIQSTADRQIFEEKVGPLVGRLRLTASLRQNGAKTAYRVNLKLDQADVVDCSTSVCGELPKVRYTQVWSHDVTIVANSTEASRKSLYDLTKSLVATSQVEDLVVNLVPLGR

SEQ ID NO: 65 - protein, 89 aa, synthetic construct
MGSMKSIRCKNCNKLLFKADSFDHIEIRCPRCKRHIIMLNACEHPTEKHCGKREKITHSDETVRYGSTSGHKLNGGGGGMDAKSLTAWS

SEQ ID NO: 66 - protein, 22 aa, synthetic construct
ESKGTAKSRYKARRAELIAERR

SEQ ID NO: 67 - protein, 30 aa, synthetic construct
ATVITYGKSTFAGNAKTRRHERRRKLAIER

SEQ ID NO: 68 - protein, 22 aa, synthetic construct
MDAQTRRRERRAEKQAQWKAAN

SEQ ID NO: 69 - RNA, 19 nt, synthetic construct
  source 1..19: mol_type = other RNA
acatgaggatcacccatgt

SEQ ID NO: 70 - RNA, 25 nt, synthetic construct
  source 1..25: mol_type = other RNA
taaggagtttatatggaaaccctta

SEQ ID NO: 71 - RNA, 19 nt, synthetic construct
  source 1..19: mol_type = other RNA
ctgaatgcctgcgagcatc

SEQ ID NO: 72 - RNA, 24 nt, synthetic construct
  source 1..24: mol_type = other RNA
ggttcacctctaaccgggtgagcc

SEQ ID NO: 73 - RNA, 16 nt, synthetic construct
  source 1..16: mol_type = other RNA
ggcgctgacaaagcgc

SEQ ID NO: 74 - RNA, 16 nt, synthetic construct
  source 1..16: mol_type = other RNA
ggccctgaaaaagggc

SEQ ID NO: 75 - protein, 495 aa, synthetic construct
MAVVYVISKSGKPLMPTTRCGHVRILLKEGKARVVERKPFTIQLTYESAEETQPLVLGIDPGRTNIGMSVVTESGESVFNAQIETRNKDVPKLMKDRKQYRMAHRRLKRRCKRRRRAKAAGTAFEEGEKQRLLPGCFKPITCKSIRNKEARFNNRKRPVGWLTPTANHLLVTHLNVVKKVQKILPVAKVVLELNRFSFMAMNNPKVQRWQYQRGPLYGKGSVEEAVSMQQDGHCLFCKHGIDHYHHVVPRRKNGSETLENRVGLCEEHHRLVHTDKEWEANLASKKSGMNKKYHALSVLNQIIPYLADQLADMFPGNFCVTSGQDTYLFREEHGIPKDHYLDAYCIACSALTDAKKVSSPKGRPYMVHQFRRHDRQACHKANLNRSYYMGGKLVATNRHKAMDQKTDSLEEYRAAHSAADVSKLTVKHPSAQYKDMSRIMPGSILVSGEGKLFTLSRSEGRNKGQVNYFVSTEGIKYWARKCQYLRNNGGLQIYV

SEQ ID NO: 76 - protein, 427 aa, Eubacterium rectale
MDTSNLMEQILSSDNLNRAYLQVVRNKGAEGVDGMKYTELKEHLAKNGETIKGQLRTRKYKPQPARRVEIPKPDGGVRNLGVPTVTDRFIQQAIAQVLTPIYEEQFHDHSYGFRPNRCAQQAILTALNIMNDGNDWIVDIDLEKFFDTVNHDKLMTLIGRTIKDGDVISIVRKYLVSGIMIDDEYEDSIVGTPQGGNLSPLLANIMLNELDKEMEKRGLNFVRYADDCIIMVGSEMSANRVMRNISRFIEEKLGLKVNMTKSKVDRPSGLKYLGFGFYFDPRAHQFKAKPHAKSVAKFKKRMKELTCRSWGVSNSYKVEKLNQLIRGWINYFKIGSMKTLCKELDSRIRYRLRMCIWKQWKTPQNQEKNLVKLGIDRNTARRVAYTGKRIAYVCNKGAVNVAISNKRLASFGLISMLDYYIEKCVTC

SEQ ID NO: 77 - protein, 597 aa, synthetic construct
MKSRKRRNRVSFLGAATVEPPKPIPLTWKTEKPVWVNQWPLPKQKLEALHLLANEQLEKGHIEPSFSPWNSPVFVIQKKSGKWRMLTDLRAVNAVIQPMGPLQPGLPSPAMIPKDWPLIIIDLKDCFFTIPLAEQDCEKFAFTIPAINNKEPATRFQWKVLPQGMLNSPTICQTFVGRALQPVREKFSDCYIIHYIDDILCAAETKDKLIDCYTFLQAEVANAGLAIASDKIQTSTPFHYLGMQIENRKIKPQKIEIRKDTLKTLNDFQKLLGDINWIRPTLGIPTYAMSNLFSILRGDSDLNSKRMLTPEATKEIKLVEEKIQSAQINRIDPLAPLQLLIFATAHSPTGIIIQNTDLVEWSFLPHSTVKTFTLYLDQIATLIGQTRLRIIKLCGNDPDKIVVPLTKEQVRQAFINSGAWQIGLANFVGIIDNHYPKTKIFQFLKLTTWILPKITRREPLENALTVFTDGSSNGKAAYTGPKERVIKTPYQSAQRAELVAVITVLQDFDQPINIISDSAYVVQATRDVETALIKYSMDDQLNQLFNLLQQTVRKRNFPFYITHIRAHTNLPGPLTKANEQADLLVSSALIKAQELHA

SEQ ID NO: 78 - protein, 420 aa, Geobacillus stearothermophilus
MALLERILARDNLITALKRVEANQGAPGIDGVSTDQLRDYIRAHWSTIHAQLLAGTYRPAPVRRVEIPKPGGGTRQLGIPTVVDRLIQQAILQELTPIFDPDFSSSSFGFRPGRNAHDAVRQAQGYIQEGYRYVVDMDLEKFFDRVNHDILMSRVARKVKDKRVLKLIRAYLQAGVMIEGVKVQTEEGTPQGGPLSPLLANILLDDLDKELEKRGLKFCRYADDCNIYVKSLRAGQRVKQSIQRFLEKTLKLKVNEEKSAVDRPWKRAFLGFSFTPERKARIRLAPRSIQRLKQRIRQLTNPNWSISMPERIHRVNQYVMGWIGYFRLVETPSVLQTIEGWIRRRLRLCQWLQWKRVRTRIRELRALGLKETAVMEIANTRKGAWRTTKTPQLHQALGKTYWTAQGLKSLTQRYFELRQG

SEQ ID NO: 79 - protein, 677 aa, synthetic construct
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSP

SEQ ID NO: 80 - protein, 8 aa, synthetic construct
GGSGGSGG

SEQ ID NO: 81 - protein, 6 aa, synthetic construct
  VARIANT 1..3: note = The amino acid residues 1-3 can be repeated one or more times.
GGSGGS

SEQ ID NO: 82 - protein, 4 aa, synthetic construct
  VARIANT 1..2: note = The amino acid residues 1-2 can be repeated one or more times.
APAP

SEQ ID NO: 83 - protein, 5 aa, synthetic construct
  REGION >5: note = The entire sequence of amino acids 1-5 can be repeated one or more times.
EAAAK

SEQ ID NO: 84 - protein, 16 aa, synthetic construct
SGSETPGTSESATPES

SEQ ID NO: 85 - protein, 24 aa, synthetic construct
SGGSSGSETPGTSESATPESSGGS

SEQ ID NO: 86 - protein, 346 aa, synthetic construct
MNIFNTNPFKAVSFVESAVKKALETSGYLIADCKYDGVRGNIVVDNVAEAAWLSRVSKFIPALEHLNGFDKRWQQLLNDDRCIFPDGFMLDGELMVKGVDFNTGSGLLRTKWVKRDNMGFHLTNVPTKLTPKGREVIDGKFEFHLDPKRLSVRLYAVMPIHIAESGEDYDVQNLLMPYHVEAMRSLLVEYFPEIEWLIAETYEVYDMDSLTELYEEKRAEGHEGLIVKDPQGIYKRGKKSGWWKLKPECEADGIIQGVNWGTEGLANEGKVIGFSVLLETGRLVDANNISRALMDEFTSNVKAHGEDFYNGWACQVNYMEATPDGSLRHPSFEKFRGTEDNPQEKM

SEQ ID NO: 87 - protein, 487 aa, synthetic construct
MILKILNEIASIGSTKQKQAILEKNKDNELLKRVYRLTYSRGLQYYIKKWPKPGIATQSFGMLTLTDMLDFIEFTLATRKLTGNAAIEELTGYITDGKKDDVEVLRRVMMRDLECGASVSIANKVWPGLIPEQPQMLASSYDEKGINKNIKFPAFAQLKADGARCFAEVRGDELDDVRLLSRAGNEYLGLDLLKEELIKMTAEARQIHPEGVLIDGELVYHEQVKKEPEGLDFLFDAYPENSKAKEFAEVAESRTASNGIANKSLKGTISEKEAQCMKFQVWDYVPLVEIYSLPAFRLKYDVRFSKLEQMTSGYDKVILIENQVVNNLDEAKVIYKKYIDQGLEGIILKNIDGLWENARSKNLYKFKEVIDVDLKIVGIYPHRKDPTKAGGFILESECGKIKVNAGSGLKDKAGVKSHELDRTRIMENQNYYIGKILECECNGWLKSDGRTDYVKLFLPIAIRLREDKTKANTFEDVFGDFHEVTGL

SEQ ID NO: 88 - protein, 358 aa, synthetic construct
MNIKTNPFKAVSFVESAIKKALDNAGYLIAEIKYDGVRGNICVDNTANSYWLSRVSKTIPALEHLNGFDVRWKRLLNDDRCFYKDGFMLDGELMVKGVDFNTGSGLLRTKWTDTKNQEFHEELFVEPIRKKDKVPFKLHTGHLHIKLYAILPLHIVESGEDCDVMTLLMQEHVKNMLPLLQEYFPEIEWQAAESYEVYDMVELQQLYEQKRAEGHEGLIVKDPMCIYKRGKKSGWWKMKPENEADGIIQGLVWGTKGLANEGKVIGFEVLLESGRLVNATNISRALMDEFTETVKEATLSQWGFFSPYGIGDNDACTINPYDGWACQISYMEETPDGSLRHPSFVMFRGTEDNPQEKM

SEQ ID NO: 89 - protein, 298 aa, synthetic construct
MAITKPLLAATLENIEDVQFPCLATPKIDGIRSVKQTQMLSRTFKPIRNSVMNRLLTELLPEGSDGEISIEGATFQDTTSAVMTGHKMYNAKFSYYWFDYVTDDPLKKYIDRVEDMKNYITVHPHILEHAQVKIIPLIPVEINNITELLQYERDVLSKGFEGVMIRKPDGKYKFGRSTLKEGILLKMKQFKDAEATIISMTALFKNTNTKTKDNFGYSKRSTHKSGKVEEDVMGSIEVDYDGVVFSIGTGFDADQRRDFWQNKESYIGKMVKFKYFEMGSKDCPRFPVFIGIRHEEDR

SEQ ID NO: 90 - protein, 315 aa, synthetic construct
MSKRDVVLDIEKGIWRGVDQNDKAVEAIIKKNGYVIVEPKIDGCRAIVGAHGVVSRSGRRFPALDGLEDRIIAKLSQSGLDSGLVLDCEMYLEGMPFSEATGRMARKTPLTKAELKCLHFAVFDATHIGVLRKSRKSHLVYDERRAMVRSLMEDCRRGDTPYFFQVAAQSCRSMEAVLRWYGYHRAMGFEGSMEKDPSLTYRNGKVAGCYKRKPEITVDGRIVGYVMGKTGKNVGRVVGYRVELEDGSGTVAATGLSEEHIQLLTCAYLNAHIDEAMPNYGRIVEVSAMERSANTLRHPSFSRFRDLASNPGVKV

SEQ ID NO: 91 - protein, 552 aa, synthetic construct
MTSLREFRKLCCDIYHASGYKEKSKLIRDFITDRDDKYLIIKLLLPGLDDRIYNMNDKQIIKLYSIIFKQSQEDMLQDLGYGYIGDTIRTFFKENTEIRPRDKSILTLEDVDSFLTTLSSVTKESHQIKLLTDIASVCTCNDLKCVVMLIDKDLKIKAGPRYVLNAISPNAYDVFRKSNNLKEIIENASKQNLDSISISVMTPINPMLAESCDSVNKAFKKFPSGMFAEVKYDGERVQVHKNNNEFAFFSRNMKPVLSHKVDYLKEYIPKAFKKATSIVLDSEIVLVDEHNVPLPFGSLGIHKKKEYKNSNMCLFVFDCLYFDGFDMTDIPLYERRSFLKDVMVEIPNRIVFSELTNISNESQLTDVLDDALTRKLEGLVLKDINGVYEPGKRRWLKIKRDYLNEGSMADSADLVVLGAYYGKGAKGGIMAVFLMGCYDDESGKWKTVTKCSGHDDNTLRVLQDQLTMVKINKDPKKIPEWLVVNKIYIPDFVVEDPKQSQIWEISGAEFTSSKSHTANGISIRFPRFTRIREDKTWKESTHLNDLVNLTKS

SEQ ID NO: 92 - protein, 487 aa, synthetic construct
MILKILNEIASIGSTKQKQAILEKNKDNELLKRVYRLTYSRGLQYYIKKWPKPGIATQSFGMLTLTDMLDFIEFTLATRKLTGNAAIEELTGYITDGKKDDVEVLRRVMMRDLECGASVSIANKVWPGLIPEQPQMLASSYDEKGINKNIKFPAFAQLAADGAACFAEVRGDELDDVRLLSRAGNEYLGLDLLKEELIKMTAEARQIHPEGVLIDGELVYHEQVKKEPEGLDFLFDAYPENSKAKEFAEVAESRTASNGIANKSLKGTISEKEAQCMKFQVWDYVPLVEIYSLPAFRLKYDVRFSKLEQMTSGYDKVILIENQVVNNLDEAKVIYKKYIDQGLEGIILKNIDGLWENARSKNLYKFKEVIDVDLKIVGIYPHRKDPTKAGGFILESECGKIKVNAGSGLKDKAGVKSHELDRTRIMENQNYYIGKILECECNGWLKSDGRTDYVKLFLPIAIRLREDKTKANTFEDVFGDFHEVTGL

SEQ ID NO: 93 - protein, 606 aa, synthetic construct
MVISYDNYVTILDEETLKAWIAKLEKAPVFAFATATDSLDNISANLVGLSFAIEPGVAAYIPVAHDYLDAPDQISRERALELLKPLLEDEKALKVGQNLKYDRGILANYGIELRGIAFDTMLESYILNSVAGRHDMDSLAERWLKHKTITFEEIAGKGKNQLTFNQIALEEAGRYAAEDADVTLQLHLKMWPDLQKHKGPLNVFENIEMPLVPVLSRIERNGVKIDPKVLHNHSEELTLRLAELEKKAHEIAGEEFNLSSTKQLQTILFEKQGIKPLKKTPGGAPSTSEEVLEELALDYPLPKVILEYRGLAKLKSTYTDKLPLMINPKTGRVHTSYHQAVTATGRLSSTDPNLQNIPVRNEEGRRIRQAFIAPEDYVIVSADYSQIELRIMAHLSRDKGLLTAFAEGKDIHRATAAEVFGLPLETVTSEQRRSAKAINFGLIYGMSAFGLARQLNIPRKEAQKYMDLYFERYPGVLEYMERTRAQAKEQGYVETLDGRRLYLPDIKSSNGARRAAAERAAINAPMQGTAADIIKRAMIAVDAWLQAEQPRVRMIMQVHDELVFEVHKDDVDAVAKQIHQLMENCTRLDVPLLVEVGSGENWDQAH

SEQ ID NO: 94 - protein, 541 aa, synthetic construct
MALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKE

SEQ ID NO: 95 - protein, 678 aa, synthetic construct
MTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSP

SEQ ID NO: 96 - protein, 335 aa, synthetic construct
MSKRKAPQETLNGGITDMLTELANFEKNVSQAIHKYNAYRKAASVIAKYPHKIKSGAEAKKLPGVGTKIAEKIDEFLATGKLRKLEKIRQDDTSSSINFLTRVSGIGPSAARKFVDEGIKTLEDLRKNEDKLNHHQRIGLKYFGDFEKRIPREEMLQMQDIVLNEVKKVDSEYIATVCGSFRRGAESSGDMDVLLTHPSFTSESTKQPKLLHQVVEQLQKVHFITDTLSKGETKFMGVCQLPSKNDEKEYPHRRIDIRLIPKDQYYCGVLYFTGSDIFNKNMRAHALEKGFTINEYTIRPLGVTGVAGEPLPVDSEKDIFDYIQWKYREPKDRSE

SEQ ID NO: 97 - protein, 406 aa, synthetic construct
MSKRKAPQETLNGGITDMLTELANFEKNVSQAIHKYNAYRKAASVIAKYPHKIKSGAEAKKLPGVGTKIAEKIDEFLATGKLRKLEKIRQDDTSSSINFLTRVSGIGPSAARKFVDEGIKTLEDLRKNEDKLNHHQRIGLKYFGDFEKRIPREEMLQMQDIVLNEVKKVDSEYIATVCGSFRRGAESSGDMDVLLTHPSFTSESTKQPKLLHQVVEQLQKVHFITDTLSKGETKFMGVCQLPSKNDEKEYPHRRIDIRLIPKDQYYCGVLYFTGSDIFNKNMRAHALEKGFTINEYTIRPLGVTGVAGEPLPVDSEKDIFDYIQWKYREPKDRSEGGGSGGGSATVKFKYKGEEKEVDISKIKKVWRVGKMISFTYDEGGGKTGRGAVSEKDAPKELLQMLEKQKK

SEQ ID NO: 98 - protein, 575 aa, synthetic construct
MKHMPRKMYSCDFETTTKVEDCRVWAYGYMNIEDHSEYKIGNSLDEFMAWVLKVQADLYFHNLKFDGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNAIQIIAEALLIQFKQGLDRMTAGSDSLKGFKDIITTKKFKKVFPTLSLGLDKEVRYAYRGGFTWLNDRFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSEGAIKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYMKEVDGKLVEGSPDDYTDIKFSVKCAGMTDKIKKEVTFENFKVGFSRKMKPKPVQVPGGVVLVDDTFTIK

SEQ ID NO: 99 - protein, 676 aa, synthetic construct
MIVSDIEANALLESVTKFHCGVIYDYSTAEYVSYRPSDFGAYLDALEAEVARGGLIVFHNGHKYDVPALTKLAKLQLNREFHLPRENCIDTLVLSRLIHSNLKDTDMGLLRSGKLPGMLEEQGEEYVDGMEWWNFNEEMMDYNVQDVVVTKALLEKLLSDKHYFPPEIDFTDVGYTTFWSESLEAVDIEHRAAWLLAKQERNGFPFDTKAIEELYVELAARRSELLRKLTETFGSWYQPKGGTEMFCHPRTGKPLPKYPRIKTPKVGGIFKKPKNKAQREGREPCELDTREYVAGAPYTPVEHVVFNPSSRDHIQKKLQEAGWVPTKYTDKGAPVVDDEVLEGVRVDDPEKQAAIDLIKEYLMIQKRIGQSAEGDKAWLRYVAEDGKIHGSVNPNGAVTGRATHAFPNLAQIPGVRSPYGEQCRAAFGAEHHLDGITGKPWVQAGIDASGLELRCLAHFMARFDNGEYAHEILNGDIHTKNQIAAELPTRDNAKTFIYGFLYGAGDEKIGQIVGAGKERGKELKKKFLENTPAIAALRESIQQTLVESSQWVAGEQQVKWKRRWIKGLDGRKVHVRSPHAALNTLLQSAGALICKLWIIKTEEMLVEKGLKHGWDGDFAYMAWVHDEIQVGCRTEEIAQVVIETAQEAMRWVGDHWNFRCLLDTEGKMGPNWAICH

SEQ ID NO: 100 - protein, 606 aa, synthetic construct
MVISYDNYVTILDEETLKAWIAKLEKAPVFAFATATDSLDNISANLVGLSFAIEPGVAAYIPVAHDYLDAPDQISRERALELLKPLLEDEKALKVGQNLKYDRGILANYGIELRGIAFDTMLESYILNSVAGRHDMDSLAERWLKHKTITFEEIAGKGKNQLTFNQIALEEAGRYAAEDADVTLQLHLKMWPDLQKHKGPLNVFENIEMPLVPVLSRIERNGVKIDPKVLHNHSEELTLRLAELEKKAHEIAGEEFNLSSTKQLQTILFEKQGIKPLKKTPGGAPSTSEEVLEELALDYPLPKVILEYRGLAKLKSTYTDKLPLMINPKTGRVHTSYHQAVTATGRLSSTDPNLQNIPVRNEEGRRIRQAFIAPEDYVIVSAAYSQIELRIMAHLSRDKGLLTAFAEGKDIHRATAAEVFGLPLETVTSEQRRSAKAINFGLIYGMSAFGLARQLNIPRKEAQKYMDLYFERYPGVLEYMERTRAQAKEQGYVETLDGRRLYLPDIKSSNGARRAAAERAAINAPMQGTAADIIKRAMIAVDAWLQAEQPRVRMIMQVHAELVFEVHKDDVDAVAKQIHQLMENCTRLDVPLLVEVGSGENWDQAH

SEQ ID NO: 101 - protein, 898 aa, synthetic construct
MKEFYISIETVGNNIVERYIDENGKERTREVEYLPTMFRHCKEESKYKDIYGKNCAPQKFPSMKDARDWMKRMEDIGLEALGMNDFKLAYISDTYGSEIVYDRKFVRVANCDIEVTGDKFPDPMKAEYEIDAITHYDSIDDRFYVFDLLNSMYGSVSKWDAKLAAKLDCEGGDEVPQEILDRVIYMPFANARDMLMEYINLWEQKRPAIFTGWNIEGFDVPYIMNRVKMILGERSMKRFSPIGRVKSKLIQNMYGSKEIYSIDGVSILDYLDLYKKFAFTNLPSFSLESVAQHETKKGKLPYDGPINKLRETNHQRYISYNIIDVESVQAIDKIRGFIDLVLSMSYYAKMPFSGVMSPIKTWDAIIFNSLKGEHKVIPQQGSHVKQSFPGAFVFEPKPIARRYIMSFDLTSMYPSIIRQVNISPETIRGQFKVHPIHEYIAGTAPKPSDEYSCSPNGWMYDKHQEGIIPKEIAKVFFQRKDWKKKMFAEEMNAEAIKKIIMKGAGSCSTKPEVERYVKFSDDFLNELSNYTESVLNSLIEECEKAATLANTNQLNRKILINSLYGALGNIHFRYYDLRNATAITIFGQVGIQWIARKINEYLNKVCGTNDEDFIAAGDTDSVYVCVDKVIEKVGLDRFKEQNDLVEFMNQFGKKKMEPMIDVAYRELCDYMNNREHLMHMDREAISCPPLGSKGVGGFWKAKKRYALNVYDMEDKRFAEPHLKIMGMETQQSSTPKAVQEALEESIRRILQEGEESVQEYYKNFEKEYRQLDYKVIAEVKTANDIAKYDDKGWPGFKCPFHIRGVLTYRRAVSGLGVAPILDGNKVMVLPLREGNPFGDKCIAWPSGTELPKEIRSDVLSWIDHSTLFQKSFVKPLAGMCESAGMDYEEKASLDFLFG

SEQ ID NO: 102 - protein, 543 aa, synthetic construct
SPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTKKTGKRSTSAAVLEALREAHPIVEKILQYRELTKLKNTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIRRREAQAFIERYFQSFPKVRAWIEKTLEEGRRRGYVETLFGRRRYVPDLEARVKSVRRKAERMAFNMPVQGTAADLMKLAMVKLFPRLEEMGARMLLQVHDELVLEAPKERAEAVARLAKEVMEGVYPLAVPLEVEVGIGEDWLSAKE

SEQ ID NO: 103 - protein, 543 aa, synthetic construct
SPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTKKTGKRSTSAAVLEALREAHPIVEKILQYRELTKLKNTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAYRLSQELAIRRREAQAFIERYFQSFPKVRAWIEKTLEEGRRRGYVETLFGRRRYVPDLEARVKSVRRKAERMAFNMPVQGTAADLMKLAMVKLFPRLEEMGARMLLQVHDELVLEAPKERAEAVARLAKEVMEGVYPLAVPLEVEVGIGEDWLSAKE

SEQ ID NO: 104 - protein, 551 aa, synthetic construct
SPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTKKTGKRSTSAAVLEALREAHPIVEKILQYRELTKLKNTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIRRREAQAFIERYFQSFPKVRAWIEKTLEEGRRRGYVETLFGRRRYVPDLEARGPGQAPRRLVKSVRRKAERMAFNMPVQGTAADLMKLAMVKLFPRLEEMGARMLLQVHDELVLEAPKERAEAVARLAKEVMEGVYPLAVPLEVEVGIGEDWLSAK

SEQ ID NO: 105 - protein, 550 aa, synthetic construct
SPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTKKTGKRSTSAAVLEALREAHPIVEKILQYRELTKLKNTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAYRLSQELAIRRREAQAFIERYFQSFPKVRAWIEKTLEEGRRRGYVETLFGRRRYVPDLEARGPGQAPRRLVKSVRRKAERMAFNMPVQGTAADLMKLAMVKLFPRLEEMGARMLLQVHDELVLEAPKERAEAVARLAKEVMEGVYPLAVPLEVEVGIGEDWLSA

SEQ ID NO: 106 - protein, 552 aa, synthetic construct
SPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTKKTGKRSTSAAVLEALREAHPIVEKILQYRELTKLKNTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARGPGQAPRRLVKSVRRKAERMAFNMPVQGTAADLMKLAMVKLFPRLEEMGARMLLQVHDELVLEAPKERAEAVARLAKEVMEGVYPLAVPLEVEVGIGEDWLSAKE

SEQ ID NO: 107 - protein, 605 aa, synthetic construct
VISYDNYVTILDEETLKAWIAKLEKAPVFAFATATDSLDNISANLVGLSFAIEPGVAAYIPVAHDYLDAPDQISRERALELLKPLLEDEKALKVGQNLKYDRGILANYGIELRGIAFDTMLESYILNSVAGRHDMDSLAERWLKHKTITFEEIAGKGKNQLTFNQIALEEAGRYAAEDADVTLQLHLKMWPDLQKHKGPLNVFENIEMPLVPVLSRIERNGVKIDPKVLHNHSEELTLRLAELEKKAHEIAGEEFNLSSTKQLQTILFEKQGIKPLKKTPGGAPSTSEEVLEELALDYPLPKVILEYRGLAKLKSTYTDKLPLMINPKTGRVHTSYHQAVTATGRLSSTDPNLQNIPVRNEEGRRIRQAFIAPEDYVIVSADYSQIELRIMAHLSRDKGLLTAFAEGKDIHRATAAEVFGLPLETVTSEQRRSAKAINFGLIYGMSAFGLARQLNIRRKEAQKYMDLYFERYPGVLEYMERTRAQAKEQGYVETLDGRRLYLPDIKSSNGARRRKAERAAINAPMQGTAADIIKRAMIAVDAWLQAEQPRVRMIMQVHDELVFEVHKDDVDAVAKQIHQLMENCTRLDVPLLVEVGSGENWDQAH

SEQ ID NO: 108 - protein, 614 aa, synthetic construct
VISYDNYVTILDEETLKAWIAKLEKAPVFAFATATDSLDNISANLVGLSFAIEPGVAAYIPVAHDYLDAPDQISRERALELLKPLLEDEKALKVGQNLKYDRGILANYGIELRGIAFDTMLESYILNSVAGRHDMDSLAERWLKHKTITFEEIAGKGKNQLTFNQIALEEAGRYAAEDADVTLQLHLKMWPDLQKHKGPLNVFENIEMPLVPVLSRIERNGVKIDPKVLHNHSEELTLRLAELEKKAHEIAGEEFNLSSTKQLQTILFEKQGIKPLKKTPGGAPSTSEEVLEELALDYPLPKVILEYRGLAKLKSTYTDKLPLMINPKTGRVHTSYHQAVTATGRLSSTDPNLQNIPVRNEEGRRIRQAFIAPEDYVIVSADYSQIELRIMAHLSRDKGLLTAFAEGKDIHRATAAEVFGLPLETVTSEQRRSAKAINFGLIYGMSAFGLARQLNIRRKEAQKYMDLYFERYPGVLEYMERTRAQAKEQGYVETLDGRRLYLPDIKSSGPGQAPRRLNGARRRKAERAAINAPMQGTAADIIKRAMIAVDAWLQAEQPRVRMIMQVHDELVFEVHKDDVDAVAKQIHQLMENCTRLDVPLLVEVGSGENWDQAH

SEQ ID NO: 109 - protein, 614 aa, synthetic construct
VISYDNYVTILDEETLKAWIAKLEKAPVFAFATATDSLDNISANLVGLSFAIEPGVAAYIPVAHDYLDAPDQISRERALELLKPLLEDEKALKVGQNLKYDRGILANYGIELRGIAFDTMLESYILNSVAGRHDMDSLAERWLKHKTITFEEIAGKGKNQLTFNQIALEEAGRYAAEDADVTLQLHLKMWPDLQKHKGPLNVFENIEMPLVPVLSRIERNGVKIDPKVLHNHSEELTLRLAELEKKAHEIAGEEFNLSSTKQLQTILFEKQGIKPLKKTPGGAPSTSEEVLEELALDYPLPKVILEYRGLAKLKSTYTDKLPLMINPKTGRVHTSYHQAVTATGRLSSTDPNLQNIPVRNEEGRRIRQAFIAPEDYVIVSADYSQIELRIMAHLSRDKGLLTAFAEGKDIHRATAAEVFGLPLETVTSEQRRSAKAINFGLIYGMSAFGLARQLNIPRKEAQKYMDLYFERYPGVLEYMERTRAQAKEQGYVETLDGRRLYLPDIKSSGPGQAPRRLNGARRRKAERAAINAPMQGTAADIIKRAMIAVDAWLQAEQPRVRMIMQVHDELVFEVHKDDVDAVAKQIHQLMENCTRLDVPLLVEVGSGENWDQAH

SEQ ID NO: 110 - protein, 605 aa, synthetic construct
VISYDNYVTILDEETLKAWIAKLEKAPVFAFATATDSLDNISANLVGLSFAIEPGVAAYIPVAHDYLDAPDQISRERALELLKPLLEDEKALKVGQNLKYDRGILANYGIELRGIAFDTMLESYILNSVAGRHDMDSLAERWLKHKTITFEEIAGKGKNQLTFNQIALEEAGRYAAEDADVTLQLHLKMWPDLQKHKGPLNVFENIEMPLVPVLSRIERNGVKIDPKVLHNHSEELTLRLAELEKKAHEIAGEEFNLSSTKQLQTILFEKQGIKPLKKTKGGAPSTSEEVLEELALDYPLPKVILEYRGLAKLKNTYTDKLPLMINPKTGRVHTSYHQAVTATGRLSSTDPNLQNIPVRNEEGRRIRQAFIAPEDYVIVSADYSQIELRIMAHLSRDKGLLTAFAEGKDIHRATAAEVFGLPLETVTSEQRRSAKAINFGLIYGMSAFGLARQLNIRRKEAQKYMDLYFERYPGVLEYMERTRAQAKEQGYVETLDGRRLYLPDIKSSNGARRRKAERAAINAPMQGTAADIIKRAMIAVDAWLQAEQPRVRMIMQVHDELVFEVHKDDVDAVAKQIHQLMENCTRLDVPLLVEVGSGENWDQAH

SEQ ID NO: 111 - protein, 614 aa, synthetic construct
VISYDNYVTILDEETLKAWIAKLEKAPVFAFATATDSLDNISANLVGLSFAIEPGVAAYIPVAHDYLDAPDQISRERALELLKPLLEDEKALKVGQNLKYDRGILANYGIELRGIAFDTMLESYILNSVAGRHDMDSLAERWLKHKTITFEEIAGKGKNQLTFNQIALEEAGRYAAEDADVTLQLHLKMWPDLQKHKGPLNVFENIEMPLVPVLSRIERNGVKIDPKVLHNHSEELTLRLAELEKKAHEIAGEEFNLSSTKQLQTILFEKQGIKPLKKTKGGAPSTSEEVLEELALDYPLPKVILEYRGLAKLKNTYTDKLPLMINPKTGRVHTSYHQAVTATGRLSSTDPNLQNIPVRNEEGRRIRQAFIAPEDYVIVSADYSQIELRIMAHLSRDKGLLTAFAEGKDIHRATAAEVFGLPLETVTSEQRRSAKAINFGLIYGMSAFGLARQLNIRRKEAQKYMDLYFERYPGVLEYMERTRAQAKEQGYVETLDGRRLYLPDIKSSGPGQAPRRLNGARRRKAERAAINAPMQGTAADIIKRAMIAVDAWLQAEQPRVRMIMQVHDELVFEVHKDDVDAVAKQIHQLMENCTRLDVPLLVEVGSGENWDQAH

SEQ ID NO: 112 - protein, 614 aa, synthetic construct
VISYDNYVTILDEETLKAWIAKLEKAPVFAFATATDSLDNISANLVGLSFAIEPGVAAYIPVAHDYLDAPDQISRERALELLKPLLEDEKALKVGQNLKYDRGILANYGIELRGIAFDTMLESYILNSVAGRHDMDSLAERWLKHKTITFEEIAGKGKNQLTFNQIALEEAGRYAAEDADVTLQLHLKMWPDLQKHKGPLNVFENIEMPLVPVLSRIERNGVKIDPKVLHNHSEELTLRLAELEKKAHEIAGEEFNLSSTKQLQTILFEKQGIKPLKKTKGGAPSTSEEVLEELALDYPLPKVILEYRGLAKLKNTYTDKLPLMINPKTGRVHTSYHQAVTATGRLSSTDPNLQNIPVRNEEGRRIRQAFIAPEDYVIVSADYSQIELRIMAHLSRDKGLLTAFAEGKDIHRATAAEVFGLPLETVTSEQRRSAKAINFGLIYGMSAFGLARQLNIPRKEAQKYMDLYFERYPGVLEYMERTRAQAKEQGYVETLDGRRLYLPDIKSSGPGQAPRRLNGARRRKAERAAINAPMQGTAADIIKRAMIAVDAWLQAEQPRVRMIMQVHDELVFEVHKDDVDAVAKQIHQLMENCTRLDVPLLVEVGSGENWDQAH

SEQ ID NO: 113 - protein, 677 aa, synthetic construct
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGLLTSEGKEIKNKDEILALLKALFLPKRLSIIHCPGHQKGHSAEARGNRMADQAARKAAITETPDTSTLLIENSSP

SEQ ID NO: 114 - protein, 604 aa, synthetic construct
VFTLWGRDIMKDIKVRLMTDSPDDSQDLMIGAIESNLFADQISWKSDQPVWLNQWPLKQEKLQALQQLVTEQLQLGHLEESNSPWNTPVFVIKKKSGKWRLLQDLRAVNATMHDMGALQPGLPSPVAVPKGWEIIIIDLQDCFFNIKLHPEDCKRFAFSVPSPNFKRPYQRFQWKVLPQGMKNSPTLCQKFVDKAILTVRDKYQDSYIVHYMDDILLAHPSRSIVDEILTSMIQALNKHGLVVSTEKIQKYDNLKYLGTHIQGDSVSYQKLQIRTDKLRTLNDFQKLLGNINWIRPFLKLTTGELKPLFEILNGDSNPISTRKLTPEACKALQLMNERLSTARVKRLDLSQPWSLCILKTEYTPTACLWQDGVVEWIHLPHISPKVITPYDIFCTQLIIKGRHRSKELFSKDPDYIVVPYTKVQFDLLLQEKEDWPISLLGFLGEVHFHLPKDPLLTFTLQTAIIFPHMTSTTPLEKGIVIFTDGSANGRSVTYIQGREPIIKENTQNTAQQAEIVAVITAFEEVSQPFNLYTDSKYVTGLFPEIETATLSPRTKIYTELKHLQRLIHKRQEKFYIGHIRGHTGLPGPLAQGNAYADSLTRILT

SEQ ID NO: 115 - protein, 572 aa, synthetic construct
TVALHLAIPLKWKPDHTPVWIDQWPLPEGKLVALTQLVEKELQLGHIEPSLSCWNTPVFVIRKASGSYRLLHDLRAVNAKLVPFGAVQQGAPVLSALPRGWPLMVLDLKDCFFSIPLAEQDREAFAFTLPSVNNQAPARRFQWKVLPQGMTCSPTICQLVVGQVLEPLRLKHPSLRMLHYMDDLLLAASSHDGLEAAGEEVISTLERAGFTISPDKIQREPGVQYLGYKLGSTYVAPVGLVAEPRIATLWDVQKLVGSLQWLRPALGIPPRLMGPFYEQLRGSDPNEAREWNLDMKMAWREIVQLSTTAALERWDPALPLEGAVARCEQGAIGVLGQGLSTHPRPCLWLFSTQPTKAFTAWLEVLTLLITKLRASAVRTFGKEVDILLLPACFREDLPLPEGILLALKGFAGKIRSSDTPSIFDIARPLHVSLKVRVTDHPVPGPTVFTDASSSTHKGVVVWREGPRWEIKEIADSGASVQQLEARAVAMALLLWPTTPTNVVTDSAFVAKMLLKMGQEGVPSTAAAFILEDALSQRSAMAAVLHVRSHSEVPGFFTEGNDVADSQATFQAY

SEQ ID NO: 116 - protein, 667 aa, synthetic construct
TLQLDDEYRLYSPQVKPDQDIQSWLEQFPQAWAETAGMGLAKQVPPQVIQLKASATPVSVRQYPLSREAREGIWPHVQRLIQQGILVPVQSPWNTPLLPVRKPGTNDYRPVQDLREVNKRVQDIHPTVPNPYNLLSALPPERNWYTVLDLKDAFFCLRLHPTSQPLFAFEWRDPGTGRTGQLTWTRLPQGFKNSPTIFDEALHRDLANFRIQHPQVTLLQYVDDLLLAGATKQDCLEGTKALLLELSDLGYRASAKKAQICRREVTYLGYSLRGGQRWLTEARKKTVVQIPAPTTAKQVREFLGTAGFCRLWIPGFATLAAPLYPLTKEKGEFSWAPEHQKAFDAIKKALLSAPALALPDVTKPFTLYVDERKGVARGVLTQTLGPWRRPVAYLSKKLDPVASGWPVCLKAIAAVAILVKDADKLTLGQNITVIAPHALENIVRQPPDRWMTNARMTHYQSLLLTERVTFAPPAALNPATLLPEETDEPVTHDCHQLLIEETGVRKDLTDIPLTGEVLTWFTDGSSYVVEGKRMAGAAVVDGTHTIWASSLPEGTSAQKAELMALTQALRLAEGKSINIYTDSRYAFATAHVHGAIYKQRGLLTSAGREIKNKEEILSLLEALHLPKRLAIIHCPGHQKAKDLISRGNQMADRVAKQAAQAVNLLPI

SEQ ID NO: 117 - protein, 693 aa, synthetic construct
APLEEEYRLFLEAPIQNVTLLEQWKREIPKVWAEINPPGLASTQAPIHVQLLSTALPVRVRQYPITLEAKRSLRETIRKFRAAGILRPVHSPWNTPLLPVRKSGTSEYRMVQDLREVNKRVETIHPTVPNPYTLLSLLPPDRIWYSVLDLKDAFFCIPLAPESQLIFAFEWADAEEGESGQLTWTRLPQGFKNSPTLFDEALNRDLQGFRLDHPSVSLLQYVDDLLIAADTQAACLSATRDLLMTLAELGYRVSGKKAQLCQEEVTYLGFKIHKGSRSLSNSRTQAILQIPVPKTKRQVREFLGTIGYCRLWIPGFAELAQPLYAATRGGNDPLVWGEKEEEAFQSLKLALTQPPALALPSLDKPFQLFVEETSGAAKGVLTQALGPWKRPVAYLSKRLDPVAAGWPRCLRAIAAAALLTREASKLTFGQDIEITSSHNLESLLRSPPDKWLTNARITQYQVLLLDPPRVRFKQTAALNPATLLPETDDTLPIHHCLDTLDSLTSTRPDLTDQPLAQAEATLFTDGSSYIRDGKRYAGAAVVTLDSVIWAEPLPIGTSAQKAELIALTKALEWSKDKSVNIYTDSRYAFATLHVHGMIYRERGLLTAGGKAIKNAPEILALLTAVWLPKRVAVMHCKGHQKDDAPTSTGNRRADEVAREVAIRPLSTQATISDAPDMPDTETPQYSNVEEALG

SEQ ID NO: 118 - protein, 689 aa, synthetic construct
VSLQDEHRLFDIPVTTSLPDVWLQDFPQAWAETGGLGRAKCQAPIIIDLKPTAVPVSIKQYPMSLEAHMGIRQHIIKFLELGVLRPCRSPWNTPLLPVKKPGTQDYRPVQDLREINKRTVDIHPTVPNPYNLLSTLKPDYSWYTVLDLKDAFFCLPLAPQSQELFAFEWKDPERGISGQLTWTRLPQGFKNSPTLFDEALHRDLTDFRTQHPEVTLLQYVDDLLLAAPTKKACTQGTRHLLQELGEKGYRASAKKAQICQTKVTYLGYILSEGKRWLTPGRIETVARIPPPRNPREVREFLGTAGFCRLWIPGFAELAAPLYALTKESTPFTWQTEHQLAFEALKKALLSAPALGLPDTSKPFTLFLDERQGIAKGVLTQKLGPWKRPVAYLSKKLDPVAAGWPPCLRIMAATAMLVKDSAKLTLGQPLTVITPHTLEAIVRQPPDRWITNARLTHYQALLLDTDRVQFGPPVTLNPATLLPVPENQPSPHDCRQVLAETHGTREDLKDQELPDADHTWYTDGSSYLDSGTRRAGAAVVDGHNTIWAQSLPPGTSAQKAELIALTKALELSKGKKANIYTDSRYAFATAHTHGSIYERRGLLTSEGKEIKNKAEIIALLKALFLPQEVAIIHCPGHQKGQDPVAVGNRQADRVARQAAMAEVLTLATEPDNTSHITIEHTYTSEDQEEA

SEQ ID NO: 119 - protein, 500 aa, synthetic construct [sequence omitted]

SEQ ID NO: 120 - protein, 526 aa, synthetic construct
MGPSAFSYVRFSSGKQAKGSSEHRQRAMLGQWLEQHPSFTLSDLRFEDLGRSGFSGEHLDHGLGQLLAAIDSGAIKSGDVILVEAVDRIGRLEPLEMLPLFSRIVKAGVSVITLEDGHVYDRSSVNETSLFLLVAKIQQAHEYSNRLSRRINASYTARREKAKAGLGIKRETPVWLTTDGQLVPHVAPHIAQAFQDYADGLGERRICRKLRESGLEEFSKTNATTVRRWLKNRTAIGYWNDIPDVYPHVVDPALFYQVQQRLDAPKVDRAKPSAHYLTGLVKCAVCGRNYNYKQRKHTDPAMLCTSRARLAGEGCSNSKTYPVIVLDQVRKLTSLPFLQHAMESASSQADPSSQRLAVIDGEIGELSRKISEATKALLVLGFTPEIQESLEQLKTAREALEEERATLLLPQAEKLTTAQLEAFSNGLLDDEPMKLNHVLQTAGYSMVVHPDGSIDVDGKRFVYEGASRKEKVYKLRLIGEDKQWSLPILTPQMATYKSLFMAAVRLPGDPSEEELRRFEEAKHSER

SEQ ID NO: 121 - protein, 535 aa, synthetic construct
MPKKKRKVYPYDVPDYAGSYRPESLDVCIYLRKSRKDVEEERRAIEEGSSYNALERHRKRLFAIAKAENHNIIDIFEEVASGESIQERPQMQQLLRKLEGNEIDGVLVIDLDRLGRGDMLDAGMIDRAFRYSSTKIITPTDVYDPDDESWELVFGIKSLISRQELKSITKRLQNGRIDSVKEGKHIGKKPPYGYLKDENLRLYPDPEKAWIVKKIFELMCDGKGRQMIAAELDRLGIDPPVTKRGAWDSSTITSIIKNEVYTGVIVWGKFKHKKRNGKYTRHKNPQEKWIMYENAHEPIISKELFDAANEAHSSRHKPAVITSKKLTNPLAGILKCKLCGYTMLIQTRKDRPHNYLRCNNPACKGKQKQSVFNLVEEKLLYSLQQIVDEYQAQKVEEVEIDDSKLISFKEKAIISKEKELKELQAQKGNLHDLLEQGIYTVEIFLERQKNLVERITSIENDIEVLQKEIETEQIKEHNKTEFIPALKTVIESYHKTTNIELKNQLLKTILSTVTYYRHPDWKTNEFEIQVYFKIS

SEQ ID NO: 122 - protein, 1909 aa, synthetic construct
MSPSKKNGRSGPQPHKRWVFTLNNPSEDERKKIRDLPISLFDYFIVGEEGNEEGRTPHLQGFANFVKKQTFNKVKWYLGARCHIEKAKGTDQQNKEYCSKEGNLLMECGAPRSQGQRSGGSSGSETPGTSESATPESSGGSDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSKRTADGSEFESPKKKRKVSGGSSGGSNIFNTNPFKAVSFVESAVKKALETSGYLIADCKYDGVRGNIVVDNVAEAAWLSRVSKFIPALEHLNGFDKRWQQLLNDDRCIFPDGFMLDGELMVKGVDFNTGSGLLRTKWVKRDNMGFHLTNVPTKLTPKGREVIDGKFEFHLDPKRLSVRLYAVMPIHIAESGEDYDVQNLLMPYHVEAMRSLLVEYFPEIEWLIAETYEVYDMDSLTELYEEKRAEGHEGLIVKDPQGIYKRGKKSGWWKLKPECEADGIIQGVNWGTEGLANEGKVIGFSVLLETGRLVDANNISRALMDEFTSNVKAHGEDFYNGWACQVNYMEATPDGSLRHPSFEKFRGTEDNPQEKMSGGSKRTADGSEFESPKKKRKV

SEQ ID NO: 123 - protein, 2050 aa, synthetic construct
MSPSKKNGRSGPQPHKRWVFTLNNPSEDERKKIRDLPISLFDYFIVGEEGNEEGRTPHLQGFANFVKKQTFNKVKWYLGARCHIEKAKGTDQQNKEYCSKEGNLLMECGAPRSQGQRSGGSSGSETPGTSESATPESSGGSDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSKRTADGSEFESPKKKRKVSGGSSGGSILKILNEIASIGSTKQKQAILEKNKDNELLKRVYRLTYSRGLQYYIKKWPKPGIATQSFGMLTLTDMLDFIEFTLATRKLTGNAAIEELTGYITDGKKDDVEVLRRVMMRDLECGASVSIANKVWPGLIPEQPQMLASSYDEKGINKNIKFPAFAQLKADGARCFAEVRGDELDDVRLLSRAGNEYLGLDLLKEELIKMTAEARQIHPEGVLIDGELVYHEQVKKEPEGLDFLFDAYPENSKAKEFAEVAESRTASNGIANKSLKGTISEKEAQCMKFQVWDYVPLVEIYSLPAFRLKYDVRFSKLEQMTSGYDKVILIENQVVNNLDEAKVIYKKYIDQGLEGIILKNIDGLWENARSKNLYKFKEVIDVDLKIVGIYPHRKDPTKAGGFILESECGKIKVNAGSGLKDKAGVKSHELDRTRIMENQNYYIGKILECECNGWLKSDGRTDYVKLFLPIAIRLREDKTKANTFEDVFGDFHEVTGLSGGSKRTADGSEFESPKKKRKV

SEQ ID NO: 124 - protein, 1921 aa, synthetic construct
MSPSKKNGRSGPQPHKRWVFTLNNPSEDERKKIRDLPISLFDYFIVGEEGNEEGRTPHLQGFANFVKKQTFNKVKWYLGARCHIEKAKGTDQQNKEYCSKEGNLLMECGAPRSQGQRSGGSSGSETPGTSESATPESSGGSDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSKRTADGSEFESPKKKRKVSGGSSGGSNIKTNPFKAVSFVESAIKKALDNAGYLIAEIKYDGVRGNICVDNTANSYWLSRVSKTIPALEHLNGFDVRWKRLLNDDRCFYKDGFMLDGELMVKGVDFNTGSGLLRTKWTDTKNQEFHEELFVEPIRKKDKVPFKLHTGHLHIKLYAILPLHIVESGEDCDVMTLLMQEHVKNMLPLLQEYFPEIEWQAAESYEVYDMVELQQLYEQKRAEGHEGLIVKDPMCIYKRGKKSGWWKMKPENEADGIIQGLVWGTKGLANEGKVIGFEVLLESGRLVNATNISRALMDEFTETVKEATLSQWGFFSPYGIGDNDACTINPYDGWACQISYMEETPDGSLRHPSFVMFRGTEDNPQEKMSGGSKRTADGSEFESPKKKRKV

SEQ ID NO: 125 - protein, 1861 aa, synthetic construct
MSPSKKNGRSGPQPHKRWVFTLNNPSEDERKKIRDLPISLFDYFIVGEEGNEEGRTPHLQGFANFVKKQTFNKVKWYLGARCHIEKAKGTDQQNKEYCSKEGNLLMECGAPRSQGQRSGGSSGSETPGTSESATPESSGGSDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSKRTADGSEFESPKKKRKVSGGSSGGSAITKPLLAATLENIEDVQFPCLATPKIDGIRSVKQTQMLSRTFKPIRNSVMNRLLTELLPEGSDGEISIEGATFQDTTSAVMTGHKMYNAKFSYYWFDYVTDDPLKKYIDRVEDMKNYITVHPHILEHAQVKIIPLIPVEINNITELLQYERDVLSKGFEGVMIRKPDGKYKFGRSTLKEGILLKMKQFKDAEATIISMTALFKNTNTKTKDNFGYSKRSTHKSGKVEEDVMGSIEVDYDGVVFSIGTGFDADQRRDFWQNKESYIGKMVKFKYFEMGSKDCPRFPVFIGIRHEEDRSGGSKRTADGSEFESPKKKRKV

SEQ ID NO: 126 - protein, 1878 aa, synthetic construct
MSPSKKNGRSGPQPHKRWVFTLNNPSEDERKKIRDLPISLFDYFIVGEEGNEEGRTPHLQGFANFVKKQTFNKVKWYLGARCHIEKAKGTDQQNKEYCSKEGNLLMECGAPRSQGQRSGGSSGSETPGTSESATPESSGGSDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSKRTADGSEFESPKKKRKVSGGSSGGSSKRDVVLDIEKGIWRGVDQNDKAVEAIIKKNGYVIVEPKIDGCRAIVGAHGVVSRSGRRFPALDGLEDRIIAKLSQSGLDSGLVLDCEMYLEGMPFSEATGRMARKTPLTKAELKCLHFAVFDATHIGVLRKSRKSHLVYDERRAMVRSLMEDCRRGDTPYFFQVAAQSCRSMEAVLRWYGYHRAMGFEGSMEKDPSLTYRNGKVAGCYKRKPEITVDGRIVGYVMGKTGKNVGRVVGYRVELEDGSGTVAATGLSEEHIQLLTCAYLNAHIDEAMPNYGRIVEVSAMERSANTLRHPSFSRFRDLASNPGVKVSGGSKRTADGSEFESPKKKRKV

SEQ ID NO: 127 - protein, 2115 aa, synthetic construct
MSPSKKNGRSGPQPHKRWVFTLNNPSEDERKKIRDLPISLFDYFIVGEEGNEEGRTPHLQGFANFVKKQTFNKVKWYLGARCHIEKAKGTDQQNKEYCSKEGNLLMECGAPRSQGQRSGGSSGSETPGTSESATPESSGGSDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSKRTADGSEFESPKKKRKVSGGSSGGSTSLREFRKLCCDIYHASGYKEKSKLIRDFITDRDDKYLIIKLLLPGLDDRIYNMNDKQIIKLYSIIFKQSQEDMLQDLGYGYIGDTIRTFFKENTEIRPRDKSILTLEDVDSFLTTLSSVTKESHQIKLLTDIASVCTCNDLKCVVMLIDKDLKIKAGPRYVLNAISPNAYDVFRKSNNLKEIIENASKQNLDSISISVMTPINPMLAESCDSVNKAFKKFPSGMFAEVKYDGERVQVHKNNNEFAFFSRNMKPVLSHKVDYLKEYIPKAFKKATSIVLDSEIVLVDEHNVPLPFGSLGIHKKKEYKNSNMCLFVFDCLYFDGFDMTDIPLYERRSFLKDVMVEIPNRIVFSELTNISNESQLTDVLDDALTRKLEGLVLKDINGVYEPGKRRWLKIKRDYLNEGSMADSADLVVLGAYYGKGAKGGIMAVFLMGCYDDESGKWKTVTKCSGHDDNTLRVLQDQLTMVKINKDPKKIPEWLVVNKIYIPDFVVEDPKQSQIWEISGAEFTSSKSHTANGISIRFPRFTRIREDKTWKESTHLNDLVNLTKSSGGSKRTADGSEFESPKKKRKV

SEQ ID NO: 128 - protein, 1932 aa, synthetic construct
MKRTADGSEFESPKKKRKVSGGSDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSKRTADGSEFESPKKKRKVSGGSSGGSILKILNEIASIGSTKQKQAILEKNKDNELLKRVYRLTYSRGLQYYIKKWPKPGIATQSFGMLTLTDMLDFIEFTLATRKLTGNAAIEELTGYITDGKKDDVEVLRRVMMRDLECGASVSIANKVWPGLIPEQPQMLASSYDEKGINKNIKFPAFAQLKADGARCFAEVRGDELDDVRLLSRAGNEYLGLDLLKEELIKMTAEARQIHPEGVLIDGELVYHEQVKKEPEGLDFLFDAYPENSKAKEFAEVAESRTASNGIANKSLKGTISEKEAQCMKFQVWDYVPLVEIYSLPAFRLKYDVRFSKLEQMTSGYDKVILIENQVVNNLDEAKVIYKKYIDQGLEGIILKNIDGLWENARSKNLYKFKEVIDVDLKIVGIYPHRKDPTKAGGFILESECGKIKVNAGSGLKDKAGVKSHELDRTRIMENQNYYIGKILECECNGWLKSDGRTDYVKLFLPIAIRLREDKTKANTFEDVFGDFHEVTGLSGGSKRTADGSEFESPKKKRKV

SEQ ID NO: 129 - protein, 2053 aa, synthetic construct
MAEAGITGTWYNQHGSTFTVTAGADGNLTGQYENRAQGTGCQNSPYTLTGRYNGTKLEWRVEWNNSTENCHSRTEWRGQYQGGAEARINTQWNLTYEGGSGPATEQGQDTFTKVKPSAASSGGSSGSETPGTSESATPESSGGSDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRKLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLKREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSKRTADGSEFESPKKKRKVSGGSSGGSILKILNEIASIGSTKQKQAILEKNKDNELLKRVYRLTYSRGLQYYIKKWPKPGIATQSFGMLTLTDMLDFIEFTLATRKLTGNAAIEELTGYITDGKKDDVEVLRRVMMRDLECGASVSIANKVWPGLIPEQPQMLASSYDEKGINKNIKFPAFAQLKADGARCFAEVRGDELDDVRLLSRAGNEYLGLDLLKEELIKMTAEARQIHPEGVLIDGELVYHEQVKKEPEGLDFLFDAYPENSKAKEFAEVAESRTASNGIANKSLKGTISEKEAQCMKFQVWDYVPLVEIYSLPAFRLKYDVRFSKLEQMTSGYDKVILIENQVVNNLDEAKVIYKKYIDQGLEGIILKNIDGLWENARSKNLYKFKEVIDVDLKIVGIYPHRKDPTKAGGFILESECGKIKVNAGSGLKDKAGVKSHELDRTRIMENQNYYIGKILECECNGWLKSDGRTDYVKLFLPIAIRLREDKTKANTFEDVFGDFHEVTGLSGGSKRTADGSEFESPKKKRKV

SEQ ID NO: 130 - protein, 2050 aa, synthetic construct
MSPSKKNGRSGPQPHKRWVFTLNNPSEDERKKIRDLPISLFDYFIVGEEGNEEGRTPHLQGFANFVKKQTFNKVKWYLGARCHIEKAKGTDQQNKEYCSKEGNLLMECGAPRSQGQRSGGSSGSETPGTSESATPESSGGSDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRKLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLKREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSKRTADGSEFESPKKKRKVSGGSSGGSILKILNEIASIGSTKQKQAILEKNKDNELLKRVYRLTYSRGLQYYIKKWPKPGIATQSFGMLTLTDMLDFIEFTLATRKLTGNAAIEELTGYITDGKKDDVEVLRRVMMRDLECGASVSIANKVWPGLIPEQPQMLASSYDEKGINKNIKFPAFAQLAADGAACFAEVRGDELDDVRLLSRAGNEYLGLDLLKEELIKMTAEARQIHPEGVLIDGELVYHEQVKKEPEGLDFLFDAYPENSKAKEFAEVAESRTASNGIANKSLKGTISEKEAQCMKFQVWDYVPLVEIYSLPAFRLKYDVRFSKLEQMTSGYDKVILIENQVVNNLDEAKVIYKKYIDQGLEGIILKNIDGLWENARSKNLYKFKEVIDVDLKIVGIYPHRKDPTKAGGFILESECGKIKVNAGSGLKDKAGVKSHELDRTRIMENQNYYIGKILECECNGWLKSDGRTDYVKLFLPIAIRLREDKTKANTFEDVFGDFHEVTGLSGGSKRTADGSEFESPKKKRKV

SEQ ID NO: 131 - protein, 2169 aa, synthetic construct
MSPSKKNGRSGPQPHKRWVFTLNNPSEDERKKIRDLPISLFDYFIVGEEGNEEGRTPHLQGFANFVKKQTFNKVKWYLGARCHIEKAKGTDQQNKEYCSKEGNLLMECGAPRSQGQRSGGSSGSETPGTSESATPESSGGSDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSKRTADGSEFESPKKKRKVSGGSSGGSVISYDNYVTILDEETLKAWIAKLEKAPVFAFATATDSLDNISANLVGLSFAIEPGVAAYIPVAHDYLDAPDQISRERALELLKPLLEDEKALKVGQNLKYDRGILANYGIELRGIAFDTMLESYILNSVAGRHDMDSLAERWLKHKTITFEEIAGKGKNQLTFNQIALEEAGRYAAEDADVTLQLHLKMWPDLQKHKGPLNVFENIEMPLVPVLSRIERNGVKIDPKVLHNHSEELTLRLAELEKKAHEIAGEEFNLSSTKQLQTILFEKQGIKPLKKTPGGAPSTSEEVLEELALDYPLPKVILEYRGLAKLKSTYTDKLPLMINPKTGRVHTSYHQAVTATGRLSSTDPNLQNIPVRNEEGRRIRQAFIAPEDYVIVSADYSQIELRIMAHLSRDKGLLTAFAEGKDIHRATAAEVFGLPLETVTSEQRRSAKAINFGLIYGMSAFGLARQLNIPRKEAQKYMDLYFERYPGVLEYMERTRAQAKEQGYVETLDGRRLYLPDIKSSNGARRAAAERAAINAPMQGTAADIIKRAMIAVDAWLQAEQPRVRMIMQVHDELVFEVHKDDVDAVAKQIHQLMENCTRLDVPLLVEVGSGENWDQAHSGGSKRTADGSEFESPKKKRKV

SEQ ID NO: 132 - protein, 2169 aa, synthetic construct
MSPSKKNGRSGPQPHKRWVFTLNNPSEDERKKIRDLPISLFDYFIVGEEGNEEGRTPHLQGFANFVKKQTFNKVKWYLGARCHIEKAKGTDQQNKEYCSKEGNLLMECGAPRSQGQRSGGSSGSETPGTSESATPESSGGSDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSKRTADGSEFESPKKKRKVSGGSSGGSVISYDNYVTILDEETLKAWIAKLEKAPVFAFATATDSLDNISANLVGLSFAIEPGVAAYIPVAHDYLDAPDQISRERALELLKPLLEDEKALKVGQNLKYDRGILANYGIELRGIAFDTMLESYILNSVAGRHDMDSLAERWLKHKTITFEEIAGKGKNQLTFNQIALEEAGRYAAEDADVTLQLHLKMWPDLQKHKGPLNVFENIEMPLVPVLSRIERNGVKIDPKVLHNHSEELTLRLAELEKKAHEIAGEEFNLSSTKQLQTILFEKQGIKPLKKTPGGAPSTSEEVLEELALDYPLPKVILEYRGLAKLKSTYTDKLPLMINPKTGRVHTSYHQAVTATGRLSSTDPNLQNIPVRNEEGRRIRQAFIAPEDYVIVSAAYSQIELRIMAHLSRDKGLLTAFAEGKDIHRATAAEVFGLPLETVTSEQRRSAKAINFGLIYGMSAFGLARQLNIPRKEAQKYMDLYFERYPGVLEYMERTRAQAKEQGYVETLDGRRLYLPDIKSSNGARRAAAERAAINAPMQGTAADIIKRAMIAVDAWLQAEQPRVRMIMQVHAELVFEVHKDDVDAVAKQIHQLMENCTRLDVPLLVEVGSGENWDQAHSGGSKRTADGSEFESPKKKRKV

SEQ ID NO: 133 - protein, 2104 aa, synthetic construct
MSPSKKNGRSGPQPHKRWVFTLNNPSEDERKKIRDLPISLFDYFIVGEEGNEEGRTPHLQGFANFVKKQTFNKVKWYLGARCHIEKAKGTDQQNKEYCSKEGNLLMECGAPRSQGQRSGGSSGSETPGTSESATPESSGGSDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSKRTADGSEFESPKKKRKVSGGSSGGSALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKESGGSKRTADGSEFESPKKKRKV

SEQ ID NO: 134 - protein, 2241 aa, synthetic construct
MSPSKKNGRSGPQPHKRWVFTLNNPSEDERKKIRDLPISLFDYFIVGEEGNEEGRTPHLQGFANFVKKQTFNKVKWYLGARCHIEKAKGTDQQNKEYCSKEGNLLMECGAPRSQGQRSGGSSGSETPGTSESATPESSGGSDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSKRTADGSEFESPKKKRKVSGGSSGGSTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSPSGGSKRTADGSEFESPKKKRKV

SEQ ID NO: 135 - protein, 1898 aa, synthetic construct
MSPSKKNGRSGPQPHKRWVFTLNNPSEDERKKIRDLPISLFDYFIVGEEGNEEGRTPHLQGFANFVKKQTFNKVKWYLGARCHIEKAKGTDQQNKEYCSKEGNLLMECGAPRSQGQRSGGSSGSETPGTSESATPESSGGSDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSKRTADGSEFESPKKKRKVSGGSSGGSSKRKAPQETLNGGITDMLTELANFEKNVSQAIHKYNAYRKAASVIAKYPHKIKSGAEAKKLPGVGTKIAEKIDEFLATGKLRKLEKIRQDDTSSSINFLTRVSGIGPSAARKFVDEGIKTLEDLRKNEDKLNHHQRIGLKYFGDFEKRIPREEMLQMQDIVLNEVKKVDSEYIATVCGSFRRGAESSGDMDVLLTHPSFTSESTKQPKLLHQVVEQLQKVHFITDTLSKGETKFMGVCQLPSKNDEKEYPHRRIDIRLIPKDQYYCGVLYFTGSDIFNKNMRAHALEKGFTINEYTIRPLGVTGVAGEPLPVDSEKDIFDYIQWKYREPKDRSESGGSKRTADGSEFESPKKKRKV

SEQ ID NO: 136 - protein, 1969 aa, synthetic construct
MSPSKKNGRSGPQPHKRWVFTLNNPSEDERKKIRDLPISLFDYFIVGEEGNEEGRTPHLQGFANFVKKQTFNKVKWYLGARCHIEKAKGTDQQNKEYCSKEGNLLMECGAPRSQGQRSGGSSGSETPGTSESATPESSGGSDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSKRTADGSEFESPKKKRKVSGGSSGGSSKRKAPQETLNGGITDMLTELANFEKNVSQAIHKYNAYRKAASVIAKYPHKIKSGAEAKKLPGVGTKIAEKIDEFLATGKLRKLEKIRQDDTSSSINFLTRVSGIGPSAARKFVDEGIKTLEDLRKNEDKLNHHQRIGLKYFGDFEKRIPREEMLQMQDIVLNEVKKVDSEYIATVCGSFRRGAESSGDMDVLLTHPSFTSESTKQPKLLHQVVEQLQKVHFITDTLSKGETKFMGVCQLPSKNDEKEYPHRRIDIRLIPKDQYYCGVLYFTGSDIFNKNMRAHALEKGFTINEYTIRPLGVTGVAGEPLPVDSEKDIFDYIQWKYREPKDRSEGGGSGGGSATVKFKYKGEEKEVDISKIKKVWRVGKMISFTYDEGGGKTGRGAVSEKDAPKELLQMLEKQKKSGGSKRTADGSEFESPKKKRKV

SEQ ID NO: 137 - protein, 2239 aa, synthetic construct
MSPSKKNGRSGPQPHKRWVFTLNNPSEDERKKIRDLPISLFDYFIVGEEGNEEGRTPHLQGFANFVKKQTFNKVKWYLGARCHIEKAKGTDQQNKEYCSKEGNLLMECGAPRSQGQRSGGSSGSETPGTSESATPESSGGSDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSKRTADGSEFESPKKKRKVSGGSSGGSIVSDIEANALLESVTKFHCGVIYDYSTAEYVSYRPSDFGAYLDALEAEVARGGLIVFHNGHKYDVPALTKLAKLQLNREFHLPRENCIDTLVLSRLIHSNLKDTDMGLLRSGKLPGMLEEQGEEYVDGMEWWNFNEEMMDYNVQDVVVTKALLEKLLSDKHYFPPEIDFTDVGYTTFWSESLEAVDIEHRAAWLLAKQERNGFPFDTKAIEELYVELAARRSELLRKLTETFGSWYQPKGGTEMFCHPRTGKPLPKYPRIKTPKVGGIFKKPKNKAQREGREPCELDTREYVAGAPYTPVEHVVFNPSSRDHIQKKLQEAGWVPTKYTDKGAPVVDDEVLEGVRVDDPEKQAAIDLIKEYLMIQKRIGQSAEGDKAWLRYVAEDGKIHGSVNPNGAVTGRATHAFPNLAQIPGVRSPYGEQCRAAFGAEHHLDGITGKPWVQAGIDASGLELRCLAHFMARFDNGEYAHEILNGDIHTKNQIAAELPTRDNAKTFIYGFLYGAGDEKIGQIVGAGKERGKELKKKFLENTPAIAALRESIQQTLVESSQWVAGEQQVKWKRRWIKGLDGRKVHVRSPHAALNTLLQSAGALICKLWIIKTEEMLVEKGLKHGWDGDFAYMAWVHDEIQVGCRTEEIAQVVIETAQEAMRWVGDHWNFRCLLDTEGKMGPNWAICHSGGSKRTADGSEFESPKKKRKV

SEQ ID NO: 138 - protein, 2138 aa, synthetic construct
MSPSKKNGRSGPQPHKRWVFTLNNPSEDERKKIRDLPISLFDYFIVGEEGNEEGRTPHLQGFANFVKKQTFNKVKWYLGARCHIEKAKGTDQQNKEYCSKEGNLLMECGAPRSQGQRSGGSSGSETPGTSESATPESSGGSDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSKRTADGSEFESPKKKRKVSGGSSGGSKHMPRKMYSCDFETTTKVEDCRVWAYGYMNIEDHSEYKIGNSLDEFMAWVLKVQADLYFHNLKFDGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNAIQIIAEALLIQFKQGLDRMTAGSDSLKGFKDIITTKKFKKVFPTLSLGLDKEVRYAYRGGFTWLNDRFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSEGAIKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYMKEVDGKLVEGSPDDYTDIKFSVKCAGMTDKIKKEVTFENFKVGFSRKMKPKPVQVPGGVVLVDDTFTIKSGGSKRTADGSEFESPKKKRKV

SEQ ID NO: 139 - protein, 2169 aa, synthetic construct
MSPSKKNGRSGPQPHKRWVFTLNNPSEDERKKIRDLPISLFDYFIVGEEGNEEGRTPHLQGFANFVKKQTFNKVKWYLGARCHIEKAKGTDQQNKEFCSKEGNLLMECGAPRSQGQRSGGSSGSETPGTSESATPESSGGSDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSKRTADGSEFESPKKKRKVSGGSSGGSVISYDNYVTILDEETLKAWIAKLEKAPVFAFATATDSLDNISANLVGLSFAIEPGVAAYIPVAHDYLDAPDQISRERALELLKPLLEDEKALKVGQNLKYDRGILANYGIELRGIAFDTMLESYILNSVAGRHDMDSLAERWLKHKTITFEEIAGKGKNQLTFNQIALEEAGRYAAEDADVTLQLHLKMWPDLQKHKGPLNVFENIEMPLVPVLSRIERNGVKIDPKVLHNHSEELTLRLAELEKKAHEIAGEEFNLSSTKQLQTILFEKQGIKPLKKTPGGAPSTSEEVLEELALDYPLPKVILEYRGLAKLKSTYTDKLPLMINPKTGRVHTSYHQAVTATGRLSSTDPNLQNIPVRNEEGRRIRQAFIAPEDYVIVSADYSQIELRIMAHLSRDKGLLTAFAEGKDIHRATAAEVFGLPLETVTSEQRRSAKAINFGLIYGMSAFGLARQLNIPRKEAQKYMDLYFERYPGVLEYMERTRAQAKEQGYVETLDGRRLYLPDIKSSNGARRAAAERAAINAPMQGTAADIIKRAMIAVDAWLQAEQPRVRMIMQVHDELVFEVHKDDVDAVAKQIHQLMENCTRLDVPLLVEVGSGENWDQAHSGGSKRTADGSEFESPKKKRKV

SEQ ID NO: 140 - protein, 2172 aa, synthetic construct
MAKSGNYSYKRWVFTINNPTFEDYVHVLEFCTLDNCKFAIVGEEKGANGTPHLQGFLNLRSNARAAALEESLGGRAWLSRARGSDEDNEEYCAKESTYLRVGEPVSKGRSSDLAEATSAVSGGSSGSETPGTSESATPESSGGSDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSKRTADGSEFESPKKKRKVSGGSSGGSVISYDNYVTILDEETLKAWIAKLEKAPVFAFATATDSLDNISANLVGLSFAIEPGVAAYIPVAHDYLDAPDQISRERALELLKPLLEDEKALKVGQNLKYDRGILANYGIELRGIAFDTMLESYILNSVAGRHDMDSLAERWLKHKTITFEEIAGKGKNQLTFNQIALEEAGRYAAEDADVTLQLHLKMWPDLQKHKGPLNVFENIEMPLVPVLSRIERNGVKIDPKVLHNHSEELTLRLAELEKKAHEIAGEEFNLSSTKQLQTILFEKQGIKPLKKTPGGAPSTSEEVLEELALDYPLPKVILEYRGLAKLKSTYTDKLPLMINPKTGRVHTSYHQAVTATGRLSSTDPNLQNIPVRNEEGRRIRQAFIAPEDYVIVSADYSQIELRIMAHLSRDKGLLTAFAEGKDIHRATAAEVFGLPLETVTSEQRRSAKAINFGLIYGMSAFGLARQLNIPRKEAQKYMDLYFERYPGVLEYMERTRAQAKEQGYVETLDGRRLYLPDIKSSNGARRAAAERAAINAPMQGTAADIIKRAMIAVDAWLQAEQPRVRMIMQVHDELVFEVHKDDVDAVAKQIHQLMENCTRLDVPLLVEVGSGENWDQAHSGGSKRTADGSEFESPKKKRKV

SEQ ID NO: 141 - protein, 2150 aa, synthetic construct
MARQVICWCFTLNNPLSPLSLHDSMKYLVYQTEQGEAGNIHFQGYIEMKKRTSLAGMKKLIPGAHFEKRRGTQGEARAYSMKEDTRLEGPWEYGEFVPSGGSSGSETPGTSESATPESSGGSDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSKRTADGSEFESPKKKRKVSGGSSGGSVISYDNYVTILDEETLKAWIAKLEKAPVFAFATATDSLDNISANLVGLSFAIEPGVAAYIPVAHDYLDAPDQISRERALELLKPLLEDEKALKVGQNLKYDRGILANYGIELRGIAFDTMLESYILNSVAGRHDMDSLAERWLKHKTITFEEIAGKGKNQLTFNQIALEEAGRYAAEDADVTLQLHLKMWPDLQKHKGPLNVFENIEMPLVPVLSRIERNGVKIDPKVLHNHSEELTLRLAELEKKAHEIAGEEFNLSSTKQLQTILFEKQGIKPLKKTPGGAPSTSEEVLEELALDYPLPKVILEYRGLAKLKSTYTDKLPLMINPKTGRVHTSYHQAVTATGRLSSTDPNLQNIPVRNEEGRRIRQAFIAPEDYVIVSADYSQIELRIMAHLSRDKGLLTAFAEGKDIHRATAAEVFGLPLETVTSEQRRSAKAINFGLIYGMSAFGLARQLNIPRKEAQKYMDLYFERYPGVLEYMERTRAQAKEQGYVETLDGRRLYLPDIKSSNGARRAAAERAAINAPMQGTAADIIKRAMIAVDAWLQAEQPRVRMIMQVHDELVFEVHKDDVDAVAKQIHQLMENCTRLDVPLLVEVGSGENWDQAHSGGSKRTADGSEFESPKKKRKV

SEQ ID NO: 142 - protein, 2182 aa, synthetic construct
MSEKKEIVKGRDWTFLVYPESAPENWRTILDETFMRWVESPLHDKDVNADGEIKKPHWHILLSSDGPITQTAVQKIIGPLNAPNAQKVGSAKGLVRYMVHLDNPEKYQYSLDEIVGHNGADVASYFELTASGGSSGSETPGTSESATPESSGGSDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSKRTADGSEFESPKKKRKVSGGSSGGSVISYDNYVTILDEETLKAWIAKLEKAPVFAFATATDSLDNISANLVGLSFAIEPGVAAYIPVAHDYLDAPDQISRERALELLKPLLEDEKALKVGQNLKYDRGILANYGIELRGIAFDTMLESYILNSVAGRHDMDSLAERWLKHKTITFEEIAGKGKNQLTFNQIALEEAGRYAAEDADVTLQLHLKMWPDLQKHKGPLNVFENIEMPLVPVLSRIERNGVKIDPKVLHNHSEELTLRLAELEKKAHEIAGEEFNLSSTKQLQTILFEKQGIKPLKKTPGGAPSTSEEVLEELALDYPLPKVILEYRGLAKLKSTYTDKLPLMINPKTGRVHTSYHQAVTATGRLSSTDPNLQNIPVRNEEGRRIRQAFIAPEDYVIVSADYSQIELRIMAHLSRDKGLLTAFAEGKDIHRATAAEVFGLPLETVTSEQRRSAKAINFGLIYGMSAFGLARQLNIPRKEAQKYMDLYFERYPGVLEYMERTRAQAKEQGYVETLDGRRLYLPDIKSSNGARRAAAERAAINAPMQGTAADIIKRAMIAVDAWLQAEQPRVRMIMQVHDELVFEVHKDDVDAVAKQIHQLMENCTRLDVPLLVEVGSGENWDQAHSGGSKRTADGSEFESPKKKRKV

SEQ ID NO: 143 - protein, 2382 aa, synthetic construct
MMSIAQVRSAGSAGNYYTDKDNYYVLGSMGERWAGRGAEQLGLQGSVDKDVFTRLLEGRLPDGADLSRMQDGSNRHRPGYDLTFSAPKSVSMMAMLGGDKRLIDAHNQAVDFAVRQVEALASTRVMTDGQSETVLTGNLVMALFNHDTSRDQEPQLHTHAVVANVTQHNGEWKTLSSDKVGKTGFIENVYANQIAFGRLYREKLKEQVEALGYETEVVGKHGMWEMPGVPVEAFSGRSQTIREAVGEDASLKSRDVAALDTRKSKQHVDPEIKMAEWMQTLKETGFDIRAYRDAADQRADLRTLTPGPASQDGPDVQQAVTQAIAGLSERSGGSSGSETPGTSESATPESSGGSDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSKRTADGSEFESPKKKRKVSGGSSGGSVISYDNYVTILDEETLKAWIAKLEKAPVFAFATATDSLDNISANLVGLSFAIEPGVAAYIPVAHDYLDAPDQISRERALELLKPLLEDEKALKVGQNLKYDRGILANYGIELRGIAFDTMLESYILNSVAGRHDMDSLAERWLKHKTITFEEIAGKGKNQLTFNQIALEEAGRYAAEDADVTLQLHLKMWPDLQKHKGPLNVFENIEMPLVPVLSRIERNGVKIDPKVLHNHSEELTLRLAELEKKAHEIAGEEFNLSSTKQLQTILFEKQGIKPLKKTPGGAPSTSEEVLEELALDYPLPKVILEYRGLAKLKSTYTDKLPLMINPKTGRVHTSYHQAVTATGRLSSTDPNLQNIPVRNEEGRRIRQAFIAPEDYVIVSADYSQIELRIMAHLSRDKGLLTAFAEGKDIHRATAAEVFGLPLETVTSEQRRSAKAINFGLIYGMSAFGLARQLNIPRKEAQKYMDLYFERYPGVLEYMERTRAQAKEQGYVETLDGRRLYLPDIKSSNGARRAAAERAAINAPMQGTAADIIKRAMIAVDAWLQAEQPRVRMIMQVHDELVFEVHKDDVDAVAKQIHQLMENCTRLDVPLLVEVGSGENWDQAHSGGSKRTADGSEFESPKKKRKV

SEQ ID NO: 144 - protein, 2169 aa, synthetic construct
MSHTSFRFRAKNVFLTYPRCPIGPEFLCDHLWNLVTPYDPLYVHVAQENHKDGGLHSHVLIQTRIEISTFDPTYFDYTGTSIPGAVVFHPNIQACRNVRDCLAYIRKNTINEVSKGASGGSSGSETPGTSESATPESSGGSDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSKRTADGSEFESPKKKRKVSGGSSGGSVISYDNYVTILDEETLKAWIAKLEKAPVFAFATATDSLDNISANLVGLSFAIEPGVAAYIPVAHDYLDAPDQISRERALELLKPLLEDEKALKVGQNLKYDRGILANYGIELRGIAFDTMLESYILNSVAGRHDMDSLAERWLKHKTITFEEIAGKGKNQLTFNQIALEEAGRYAAEDADVTLQLHLKMWPDLQKHKGPLNVFENIEMPLVPVLSRIERNGVKIDPKVLHNHSEELTLRLAELEKKAHEIAGEEFNLSSTKQLQTILFEKQGIKPLKKTPGGAPSTSEEVLEELALDYPLPKVILEYRGLAKLKSTYTDKLPLMINPKTGRVHTSYHQAVTATGRLSSTDPNLQNIPVRNEEGRRIRQAFIAPEDYVIVSADYSQIELRIMAHLSRDKGLLTAFAEGKDIHRATAAEVFGLPLETVTSEQRRSAKAINFGLIYGMSAFGLARQLNIPRKEAQKYMDLYFERYPGVLEYMERTRAQAKEQGYVETLDGRRLYLPDIKSSNGARRAAAERAAINAPMQGTAADIIKRAMIAVDAWLQAEQPRVRMIMQVHDELVFEVHKDDVDAVAKQIHQLMENCTRLDVPLLVEVGSGENWDQAHSGGSKRTADGSEFESPKKKRKV

SEQ ID NO: 145 - protein, 2176 aa, synthetic construct
MPSHPKRFQINAKNYFLTYPQCSLSKEESLSQLQALNTPINKKFIKICRELHEDGQPHLHVLIQFEGKYCCQNQRFFDLVSPTRSAHFHPNIQRAKSSSDVKTYIDKDGDTLVWGEFQVDGRSASGGSSGSETPGTSESATPESSGGSDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSKRTADGSEFESPKKKRKVSGGSSGGSVISYDNYVTILDEETLKAWIAKLEKAPVFAFATATDSLDNISANLVGLSFAIEPGVAAYIPVAHDYLDAPDQISRERALELLKPLLEDEKALKVGQNLKYDRGILANYGIELRGIAFDTMLESYILNSVAGRHDMDSLAERWLKHKTITFEEIAGKGKNQLTFNQIALEEAGRYAAEDADVTLQLHLKMWPDLQKHKGPLNVFENIEMPLVPVLSRIERNGVKIDPKVLHNHSEELTLRLAELEKKAHEIAGEEFNLSSTKQLQTILFEKQGIKPLKKTPGGAPSTSEEVLEELALDYPLPKVILEYRGLAKLKSTYTDKLPLMINPKTGRVHTSYHQAVTATGRLSSTDPNLQNIPVRNEEGRRIRQAFIAPEDYVIVSADYSQIELRIMAHLSRDKGLLTAFAEGKDIHRATAAEVFGLPLETVTSEQRRSAKAINFGLIYGMSAFGLARQLNIPRKEAQKYMDLYFERYPGVLEYMERTRAQAKEQGYVETLDGRRLYLPDIKSSNGARRAAAERAAINAPMQGTAADIIKRAMIAVDAWLQAEQPRVRMIMQVHDELVFEVHKDDVDAVAKQIHQLMENCTRLDVPLLVEVGSGENWDQAHSGGSKRTADGSEFESPKKKRKV

SEQ ID NO: 146 - protein, 2323 aa, synthetic construct
MHLFTDVVSVDIMTTMQRTHTNAELIRWFKIFRDLDIHKWVIGLEEGKGGYGHWQVRCNVRVEVVNDWTAYLRAVFGWLGPISIWTEECSDKYTYETKEGKYWASWDTMGARQQRFGKMRWNQEGAVQALQRTNDREIVVWYDEQGNMGKSWLCGHLFETGQAYYIPPYMTSIQSMIQTVASLVLQDRDSGYPPRPLIVIDIPRSWKWSTELYTAIEAIKDGLIMDPRYGARPVNIHGIKVIVLTNTKPKLDKLSEDRWVLYDPMDYLMMLSGGSSGSETPGTSESATPESSGGSDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSKRTADGSEFESPKKKRKVSGGSSGGSVISYDNYVTILDEETLKAWIAKLEKAPVFAFATATDSLDNISANLVGLSFAIEPGVAAYIPVAHDYLDAPDQISRERALELLKPLLEDEKALKVGQNLKYDRGILANYGIELRGIAFDTMLESYILNSVAGRHDMDSLAERWLKHKTITFEEIAGKGKNQLTFNQIALEEAGRYAAEDADVTLQLHLKMWPDLQKHKGPLNVFENIEMPLVPVLSRIERNGVKIDPKVLHNHSEELTLRLAELEKKAHEIAGEEFNLSSTKQLQTILFEKQGIKPLKKTPGGAPSTSEEVLEELALDYPLPKVILEYRGLAKLKSTYTDKLPLMINPKTGRVHTSYHQAVTATGRLSSTDPNLQNIPVRNEEGRRIRQAFIAPEDYVIVSADYSQIELRIMAHLSRDKGLLTAFAEGKDIHRATAAEVFGLPLETVTSEQRRSAKAINFGLIYGMSAFGLARQLNIPRKEAQKYMDLYFERYPGVLEYMERTRAQAKEQGYVETLDGRRLYLPDIKSSNGARRAAAERAAINAPMQGTAADIIKRAMIAVDAWLQAEQPRVRMIMQVHDELVFEVHKDDVDAVAKQIHQLMENCTRLDVPLLVEVGSGENWDQAHSGGSKRTADGSEFESPKKKRKV

SEQ ID NO: 147 - protein, 2323 aa, synthetic construct
MHLFTEGISVDIMTTMQRTHTNAELIRWFKIFRDLDIHKWVIGLEEGKGEYGHWQVRCNVRVEVVNDWTAYLRAVFGWLGPISIWTEECSDKYTYETKEGKYWASWDTMGARQQRFGKMRWNQEGAVQALQRTNDREIVVWYDEGGNMGKSWLCGHLFETGQAYYIPPYMTSIQSMIQTVASLVLQDRETGYPPRPLIVIDIPRSWKWSTELYTAIEAIKGGLIMDPRYGARPVNIHGTKVIVLTNTKPKLDKLSEDRWVLYDPMDYLMMLSGGSSGSETPGTSESATPESSGGSDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSKRTADGSEFESPKKKRKVSGGSSGGSVISYDNYVTILDEETLKAWIAKLEKAPVFAFATATDSLDNISANLVGLSFAIEPGVAAYIPVAHDYLDAPDQISRERALELLKPLLEDEKALKVGQNLKYDRGILANYGIELRGIAFDTMLESYILNSVAGRHDMDSLAERWLKHKTITFEEIAGKGKNQLTFNQIALEEAGRYAAEDADVTLQLHLKMWPDLQKHKGPLNVFENIEMPLVPVLSRIERNGVKIDPKVLHNHSEELTLRLAELEKKAHEIAGEEFNLSSTKQLQTILFEKQGIKPLKKTPGGAPSTSEEVLEELALDYPLPKVILEYRGLAKLKSTYTDKLPLMINPKTGRVHTSYHQAVTATGRLSSTDPNLQNIPVRNEEGRRIRQAFIAPEDYVIVSADYSQIELRIMAHLSRDKGLLTAFAEGKDIHRATAAEVFGLPLETVTSEQRRSAKAINFGLIYGMSAFGLARQLNIPRKEAQKYMDLYFERYPGVLEYMERTRAQAKEQGYVETLDGRRLYLPDIKSSNGARRAAAERAAINAPMQGTAADIIKRAMIAVDAWLQAEQPRVRMIMQVHDELVFEVHKDDVDAVAKQIHQLMENCTRLDVPLLVEVGSGENWDQAHSGGSKRTADGSEFESPKKKRKV

SEQ ID NO: 148 - protein, 2215 aa, synthetic construct
MGARQQRFGKMRWNQEGALQALQRTNDREIVVWYDQDGNMGKSWLCGHLYETGQAYYIPPYMTSIQSMIQTVASLVLQDRESGYPPRPLIVIDIPRSRKWSTELYTAIEAIKDGLIMDPRYGARPVNIHGTKVIVLTNTKPKLDKLSEDRWVLYDPLLYMMGHSGGSSGSETPGTSESATPESSGGSDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSKRTADGSEFESPKKKRKVSGGSSGGSVISYDNYVTILDEETLKAWIAKLEKAPVFAFATATDSLDNISANLVGLSFAIEPGVAAYIPVAHDYLDAPDQISRERALELLKPLLEDEKALKVGQNLKYDRGILANYGIELRGIAFDTMLESYILNSVAGRHDMDSLAERWLKHKTITFEEIAGKGKNQLTFNQIALEEAGRYAAEDADVTLQLHLKMWPDLQKHKGPLNVFENIEMPLVPVLSRIERNGVKIDPKVLHNHSEELTLRLAELEKKAHEIAGEEFNLSSTKQLQTILFEKQGIKPLKKTPGGAPSTSEEVLEELALDYPLPKVILEYRGLAKLKSTYTDKLPLMINPKTGRVHTSYHQAVTATGRLSSTDPNLQNIPVRNEEGRRIRQAFIAPEDYVIVSADYSQIELRIMAHLSRDKGLLTAFAEGKDIHRATAAEVFGLPLETVTSEQRRSAKAINFGLIYGMSAFGLARQLNIPRKEAQKYMDLYFERYPGVLEYMERTRAQAKEQGYVETLDGRRLYLPDIKSSNGARRAAAERAAINAPMQGTAADIIKRAMIAVDAWLQAEQPRVRMIMQVHDELVFEVHKDDVDAVAKQIHQLMENCTRLDVPLLVEVGSGENWDQAHSGGSKRTADGSEFESPKKKRKV

SEQ ID NO: 149 - protein, 671 aa, synthetic construct
MVISYDNYVTILDEETLKAWIAKLEKAPVFAFATATDSLDNISANLVGLSFAIEPGVAAYIPVAHDYLDAPDQISRERALELLKPLLEDEKALKVGQNLKYDRGILANYGIELRGIAFDTMLESYILNSVAGRHDMDSLAERWLKHKTITFEEIAGKGKNQLTFNQIALEEAGRYAAEDADVTLQLHLKMWPDLQKHKGPLNVFENIEMPLVPVLSRIERNGVKIDPKVLHNHSEELTLRLAELEKKAHEIAGEEFNLSSTKQLQTILFEKQGIKPLKKTPGGAPSTSEEVLEELALDYPLPKVILEYRGLAKLKSTYTDKLPLMINPKTGRVHTSYHQAVTATGRLSSTDPNLQNIPVRNEEGRRIRQAFIAPEDYVIVSADYSQIELRIMAHLSRDKGLLTAFAEGKDIHRATAAEVFGLPLETVTSEQRRSAKAINFGLIYGMSAFGLARQLNIPRKEAQKYMDLYFERYPGVLEYMERTRAQAKEQGYVETLDGRRLYLPDIKSSNGARRAAAERAAINAPMQGTAADIIKRAMIAVDAWLQAEQPRVRMIMQVHDELVFEVHKDDVDAVAKQIHQLMENCTRLDVPLLVEVGSGENWDQAHSGGSSGGSKRTADGSEFESPKKKRKVSGGSSGGSGEIAALEAKIAALKAKNAALKAEIAALEAGY

SEQ ID NO: 150 - protein, 1534 aa, synthetic construct
MSPSKKNGRSGPQPHKRWVFTLNNPSEDERKKIRDLPISLFDYFIVGEEGNEEGRTPHLQGFANFVKKQTFNKVKWYLGARCHIEKAKGTDQQNKEYCSKEGNLLMECGAPRSQGQRSGGSSGSETPGTSESATPESSGGSDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSKRTADGSEFESPKKKRKV

SEQ ID NO: 151 - protein, 1571 aa, synthetic construct
MSPSKKNGRSGPQPHKRWVFTLNNPSEDERKKIRDLPISLFDYFIVGEEGNEEGRTPHLQGFANFVKKQTFNKVKWYLGARCHIEKAKGTDQQNKEYCSKEGNLLMECGAPRSQGQRSGGSSGSETPGTSESATPESSGGSDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSKRTADGSEFESPKKKRKVSGGSSGGSKIAALKAEIAALEAENAALEAKIAALKAG

SEQ ID NO: 152 - protein, 601 aa, synthetic construct
MKHMPRKMYSCDFETTTKVEDCRVWAYGYMNIEDHSEYKIGNSLDEFMAWVLKVQADLYFHNLKFDGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNAIQIIAEALLIQFKQGLDRMTAGSDSLKGFKDIITTKKFKKVFPTLSLGLDKEVRYAYRGGFTWLNDRFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSEGAIKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYMKEVDGKLVEGSPDDYTDIKFSVKCAGMTDKIKKEVTFENFKVGFSRKMKPKPVQVPGGVVLVDDTFTIKSGGSSGGSKRTADGSEFESPKKKRKV

SEQ ID NO: 153 - protein, 640 aa, synthetic construct
MKHMPRKRYSCDFETTTKVEDCRVWAYGYMNIEDHSEYKIGNSLDEFMAWALKVQADLYFHNLKFDGAFIINWLERNGFKWSADGLPNTYNTIISRTGQWYMIDICLGYKGKRKIHTVIYDSLKKLPFPVKKIAKDFKLTVLKGDIDYHKERPVGYKITPEEYAYIKNAIQIIAEALLIQFKQGLDRMTAGSDSLKDFKDIITTKKFKKVFPTLSLGLDKKVRYAYRGGFTWLNDRFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSEGAIKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRPKTYIQDIYMKEVDGELVEGSPDDYTDIKLSVKCAGMTDKIKKEVTFENFKVGFSRKMKPKPVQVPGGVVLVDDTFTIKSGGSSGGSKRTADGSEFESPKKKRKVSGGSSGGSGEIAALEAKIAALKAKNAALKAEIAALEAGY

SEQ ID NO: 154 - protein, 640 aa, synthetic construct
MKHMPRKMYSCDFETTTKVEDCRVWAYGYMNIEDHSEYKIGNSLDEFMAWVLKVQADLYFHNLKFDGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNAIQIIAEALLIQFKQGLDRMTAGSDSLKGFKDIITTKKFKKVFPTLSLGLDKEVRYAYRGGFTWLNDRFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSEGAIKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYMKEVDGKLVEGSPDDYTDIKFSVKCAGMTDKIKKEVTFENFKVGFSRKMKPKPVQVPGGVVLVDDTFTIKSGGSSGGSKRTADGSEFESPKKKRKVSGGSSGGSGEIAALEAKIAALKAKNAALKAEIAALEAGY

SEQ ID NO: 155 - protein, 601 aa, synthetic construct
MKHMPRKMYSCDFETTTKVEDCRVWAYGYMNIEDHSEYKIGNSLDEFMAWVLKVQADLYFHNLKFDGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKGFKDIITTKKFKKVFPTLSLGLDKEVRYAYRGGFTWLNDRFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSEGAIKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYMKEVDGKLVEGSPDDYTDIKFSVKCAGMTDKIKKEVTFENFKVGFSRKMKPKPVQVPGGVVLVDDTFTIKSGGSSGGSKRTADGSEFESPKKKRKV

SEQ ID NO: 156 - protein, 640 aa, synthetic construct
MKHMPRKMYSCDFETTTKVEDCRVWAYGYMNIEDHSEYKIGNSLDEFMAWVLKVQADLYFHNLKFDGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKGFKDIITTKKFKKVFPTLSLGLDKEVRYAYRGGFTWLNDRFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSEGAIKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYMKEVDGKLVEGSPDDYTDIKFSVKCAGMTDKIKKEVTFENFKVGFSRKMKPKPVQVPGGVVLVDDTFTIKSGGSSGGSKRTADGSEFESPKKKRKVSGGSSGGSGEIAALEAKIAALKAKNAALKAEIAALEAGY

SEQ ID NO: 157 - protein, 601 aa, synthetic construct
MKHMPRKRYSCDFETTTKVEDCRVWAYGYMNIEDHSEYKIGNSLDEFMAWALKVQADLYFHNLKFDGAFIINWLERNGFKWSADGLPNTYNTIISRTGQWYMIDICLGYKGKRKIHTVIYDSLKKLPFPVKKIAKDFKLTVLKGDIDYHKERPVGYKITPEEYAYIKNAIQIIAEALLIQFKQGLDRMTAGSDSLKDFKDIITTKKFKKVFPTLSLGLDKKVRYAYRGGFTWLNDRFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSEGAIKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRPKTYIQDIYMKEVDGELVEGSPDDYTDIKLSVKCAGMTDKIKKEVTFENFKVGFSRKMKPKPVQVPGGVVLVDDTFTIKSGGSSGGSKRTADGSEFESPKKKRKV

SEQ ID NO: 158 - protein, 598 aa, synthetic construct
MPRKMFSCDFETTTKLDDCRVWAYGYMEIGNLDNYKIGNSLDEFMQWVMEIQADLYFHNLKFDGAFIVNWLERHGFKWSNEGLPNTYNTIISKMGQWYMIDICFGYKGKRKLHTVIYDSLKKLPFPVKKIAKDFQLPLLKGDIDYHKERPVGHEITPEEYEYIKNDIEIIARALDIQFKQGLDRMTAGSDSLKGFKDILSTKKFNKVFPKLSLPMDKEIRYAYRGGFTWLNDKYKEKEIGEGMVFDVNSLYPSQMYSRPLPYGAPIVFQGKYEKDEQYPLYIQRIRFEFELKEGYIPTIQIKKNPFFKGNEYLKNSGGEPVELYLTNVDLELIQEHYELYNVEYIDGFKFREKTGLFKDFIDKWTYVKTHEEGAKKQLAKLMLNSLYGKFASNPDVTGKVPYLKEDGSLGFRVGDEEYKDPVYTPMGVFITAWARFTTITAAQACYDRIIYCDTDSIHLTGTEVPEIIKDIVDPKKLGYWAHESTFKRAKYLRQKTYIQDIYMKEVDGKLVECSPDEATTTKFSVKCAGMTDTIKKKVTFDNFKVGFSSKGKPKPVQVNGGVVLVDSVFTIKSGGSSGGSKRTADGSEFESPKKKRKV

SEQ ID NO: 159 - protein, 2138 aa, synthetic construct
MSPSKKNGRSGPQPHKRWVFTLNNPSEDERKKIRDLPISLFDYFIVGEEGNEEGRTPHLQGFANFVKKQTFNKVKWYLGARCHIEKAKGTDQQNKEYCSKEGNLLMECGAPRSQGQRSGGSSGSETPGTSESATPESSGGSDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRKLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLKREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSKRTADGSEFESPKKKRKVSGGSSGGSKHMPRKMYSCDFETTTKVEDCRVWAYGYMNIEDHSEYKIGNSLDEFMAWVLKVQADLYFHNLKFDGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKGFKDIITTKKFKKVFPTLSLGLDKEVRYAYRGGFTWLNDRFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSEGAIKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYMKEVDGKLVEGSPDDYTDIKFSVKCAGMTDKIKKEVTFENFKVGFSRKMKPKPVQVPGGVVLVDDTFTIKSGGSKRTADGSEFESPKKKRKV

SEQ ID NO: 160 - protein, 637 aa, synthetic construct
MPRKMFSCDFETTTKLDDCRVWAYGYMEIGNLDNYKIGNSLDEFMQWVMEIQADLYFHNLKFDGAFIVNWLERHGFKWSNEGLPNTYNTIISKMGQWYMIDICFGYKGKRKLHTVIYDSLKKLPFPVKKIAKDFQLPLLKGDIDYHKERPVGHEITPEEYEYIKNDIEIIARALDIQFKQGLDRMTAGSDSLKGFKDILSTKKFNKVFPKLSLPMDKEIRYAYRGGFTWLNDKYKEKEIGEGMVFDVNSLYPSQMYSRPLPYGAPIVFQGKYEKDEQYPLYIQRIRFEFELKEGYIPTIQIKKNPFFKGNEYLKNSGGEPVELYLTNVDLELIQEHYELYNVEYIDGFKFREKTGLFKDFIDKWTYVKTHEEGAKKQLAKLMLNSLYGKFASNPDVTGKVPYLKEDGSLGFRVGDEEYKDPVYTPMGVFITAWARFTTITAAQACYDRIIYCDTDSIHLTGTEVPEIIKDIVDPKKLGYWAHESTFKRAKYLRQKTYIQDIYMKEVDGKLVECSPDEATTTKFSVKCAGMTDTIKKKVTFDNFKVGFSSKGKPKPVQVNGGVVLVDSVFTIKSGGSSGGSKRTADGSEFESPKKKRKVSGGSSGGSGEIAALEAKIAALKAKNAALKAEIAALEAGY

SEQ ID NO: 161 - protein, 2135 aa, synthetic construct
MSPSKKNGRSGPQPHKRWVFTLNNPSEDERKKIRDLPISLFDYFIVGEEGNEEGRTPHLQGFANFVKKQTFNKVKWYLGARCHIEKAKGTDQQNKEYCSKEGNLLMECGAPRSQGQRSGGSSGSETPGTSESATPESSGGSDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRKLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLKREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSKRTADGSEFESPKKKRKVSGGSSGGSPRKMFSCDFETTTKLDDCRVWAYGYMEIGNLDNYKIGNSLDEFMQWVMEIQADLYFHNLKFDGAFIVNWLERHGFKWSNEGLPNTYNTIISKMGQWYMIDICFGYKGKRKLHTVIYDSLKKLPFPVKKIAKDFQLPLLKGDIDYHKERPVGHEITPEEYEYIKNDIEIIARALDIQFKQGLDRMTAGSDSLKGFKDILSTKKFNKVFPKLSLPMDKEIRYAYRGGFTWLNDKYKEKEIGEGMVFDVNSLYPSQMYSRPLPYGAPIVFQGKYEKDEQYPLYIQRIRFEFELKEGYIPTIQIKKNPFFKGNEYLKNSGGEPVELYLTNVDLELIQEHYELYNVEYIDGFKFREKTGLFKDFIDKWTYVKTHEEGAKKQLAKLMLNSLYGKFASNPDVTGKVPYLKEDGSLGFRVGDEEYKDPVYTPMGVFITAWARFTTITAAQACYDRIIYCDTDSIHLTGTEVPEIIKDIVDPKKLGYWAHESTFKRAKYLRQKTYIQDIYMKEVDGKLVECSPDEATTTKFSVKCAGMTDTIKKKVTFDNFKVGFSSKGKPKPVQVNGGVVLVDSVFTIKSGGSKRTADGSEFESPKKKRKV

SEQ ID NO: 162 - protein, 640 aa, synthetic construct
MKHMPRKRYSCDFETTTKVEDCRVWAYGYMNIEDHSEYKIGNSLDEFMAWALKVQADLYFHNLKFDGAFIINWLERNGFKWSADGLPNTYNTIISRTGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKDFKDIITTKKFKKVFPTLSLGLDKKVRYAYRGGFTWLNDRFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSEGAIKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRPKTYIQDIYMKEVDGELVEGSPDDYTDIKLSVKCAGMTDKIKKEVTFENFKVGFSRKMKPKPVQVPGGVVLVDDTFTIKSGGSSGGSKRTADGSEFESPKKKRKVSGGSSGGSGEIAALEAKIAALKAKNAALKAEIAALEAGY

SEQ ID NO: 163 - protein, 2138 aa, synthetic construct
MSPSKKNGRSGPQPHKRWVFTLNNPSEDERKKIRDLPISLFDYFIVGEEGNEEGRTPHLQGFANFVKKQTFNKVKWYLGARCHIEKAKGTDQQNKEYCSKEGNLLMECGAPRSQGQRSGGSSGSETPGTSESATPESSGGSDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRKLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLKREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSKRTADGSEFESPKKKRKVSGGSSGGSKHMPRKRYSCDFETTTKVEDCRVWAYGYMNIEDHSEYKIGNSLDEFMAWALKVQADLYFHNLKFDGAFIINWLERNGFKWSADGLPNTYNTIISRTGQWYMIDICLGYKGKRKIHTVIYDSLKKLPFPVKKIAKDFKLTVLKGDIDYHKERPVGYKITPEEYAYIKNAIQIIAEALLIQFKQGLDRMTAGSDSLKDFKDIITTKKFKKVFPTLSLGLDKKVRYAYRGGFTWLNDRFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSEGAIKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRPKTYIQDIYMKEVDGELVEGSPDDYTDIKLSVKCAGMTDKIKKEVTFENFKVGFSRKMKPKPVQVPGGVVLVDDTFTIKSGGSKRTADGSEFESPKKKRKV

SEQ ID NO: 164 - protein, 6 aa, synthetic construct
HHHHHH

SEQ ID NO: 165 - protein, 601 aa, synthetic construct
MKHMPRKRYSCDFETTTKVEDCRVWAYGYMNIEDHSEYKIGNSLDEFMAWALKVQADLYFHNLKFDGAFIINWLERNGFKWSADGLPNTYNTIISRTGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKDFKDIITTKKFKKVFPTLSLGLDKKVRYAYRGGFTWLNDRFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSEGAIKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRPKTYIQDIYMKEVDGELVEGSPDDYTDIKLSVKCAGMTDKIKKEVTFENFKVGFSRKMKPKPVQVPGGVVLVDDTFTIKSGGSSGGSKRTADGSEFESPKKKRKV

SEQ ID NO: 166 - protein, 2172 aa, synthetic construct
MSPEDHTRPGTIPSKEGSGCRRWCFTLNNPTDGEIEFVRSLGPDEFYYAIVGREKGEQGTPHLQGYFHFKNKKRLSALKKMLPRGHFERAKGSDADNEKYCSKEGDVILTLGIVARDGHRSGGSSGSETPGTSESATPESSGGSDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSKRTADGSEFESPKKKRKVSGGSSGGSVISYDNYVTILDEETLKAWIAKLEKAPVFAFATATDSLDNISANLVGLSFAIEPGVAAYIPVAHDYLDAPDQISRERALELLKPLLEDEKALKVGQNLKYDRGILANYGIELRGIAFDTMLESYILNSVAGRHDMDSLAERWLKHKTITFEEIAGKGKNQLTFNQIALEEAGRYAAEDADVTLQLHLKMWPDLQKHKGPLNVFENIEMPLVPVLSRIERNGVKIDPKVLHNHSEELTLRLAELEKKAHEIAGEEFNLSSTKQLQTILFEKQGIKPLKKTPGGAPSTSEEVLEELALDYPLPKVILEYRGLAKLKSTYTDKLPLMINPKTGRVHTSYHQAVTATGRLSSTDPNLQNIPVRNEEGRRIRQAFIAPEDYVIVSADYSQIELRIMAHLSRDKGLLTAFAEGKDIHRATAAEVFGLPLETVTSEQRRSAKAINFGLIYGMSAFGLARQLNIPRKEAQKYMDLYFERYPGVLEYMERTRAQAKEQGYVETLDGRRLYLPDIKSSNGARRAAAERAAINAPMQGTAADIIKRAMIAVDAWLQAEQPRVRMIMQVHDELVFEVHKDDVDAVAKQIHQLMENCTRLDVPLLVEVGSGENWDQAHSGGSKRTADGSEFESPKKKRKV

SEQ ID NO: 167 - protein, 2187 aa, synthetic construct
MAPCKPGSNPPKGRVSAAEGGARREATRRPPREAAAKRWCFTLNNPTEEEIKSLETWLVSDFHYAIVGKEVGEQGTPHLQGFVHLKQKKRLPQLKQLFKRAHWEKARGSDEDNEKYCSKEGNVLLTLGIPAKGNRSGGSSGSETPGTSESATPESSGGSDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSKRTADGSEFESPKKKRKVSGGSSGGSVISYDNYVTILDEETLKAWIAKLEKAPVFAFATATDSLDNISANLVGLSFAIEPGVAAYIPVAHDYLDAPDQISRERALELLKPLLEDEKALKVGQNLKYDRGILANYGIELRGIAFDTMLESYILNSVAGRHDMDSLAERWLKHKTITFEEIAGKGKNQLTFNQIALEEAGRYAAEDADVTLQLHLKMWPDLQKHKGPLNVFENIEMPLVPVLSRIERNGVKIDPKVLHNHSEELTLRLAELEKKAHEIAGEEFNLSSTKQLQTILFEKQGIKPLKKTPGGAPSTSEEVLEELALDYPLPKVILEYRGLAKLKSTYTDKLPLMINPKTGRVHTSYHQAVTATGRLSSTDPNLQNIPVRNEEGRRIRQAFIAPEDYVIVSADYSQIELRIMAHLSRDKGLLTAFAEGKDIHRATAAEVFGLPLETVTSEQRRSAKAINFGLIYGMSAFGLARQLNIPRKEAQKYMDLYFERYPGVLEYMERTRAQAKEQGYVETLDGRRLYLPDIKSSNGARRAAAERAAINAPMQGTAADIIKRAMIAVDAWLQAEQPRVRMIMQVHDELVFEVHKDDVDAVAKQIHQLMENCTRLDVPLLVEVGSGENWDQAHSGGSKRTADGSEFESPKKKRKV

SEQ ID NO: 168 - protein, 2161 aa, synthetic construct
MPKQARESPCKRWCFTLNNPTEEEIERVKNLSPSEYHYAIVGKEKGEQGTPHLQGFLHLKKKQRLKQMKELIPRAHFERARGSDEDNEQYCGKEGDVILTIGVPSKGNRSGGSSGSETPGTSESATPESSGGSDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSKRTADGSEFESPKKKRKVSGGSSGGSVISYDNYVTILDEETLKAWIAKLEKAPVFAFATATDSLDNISANLVGLSFAIEPGVAAYIPVAHDYLDAPDQISRERALELLKPLLEDEKALKVGQNLKYDRGILANYGIELRGIAFDTMLESYILNSVAGRHDMDSLAERWLKHKTITFEEIAGKGKNQLTFNQIALEEAGRYAAEDADVTLQLHLKMWPDLQKHKGPLNVFENIEMPLVPVLSRIERNGVKIDPKVLHNHSEELTLRLAELEKKAHEIAGEEFNLSSTKQLQTILFEKQGIKPLKKTPGGAPSTSEEVLEELALDYPLPKVILEYRGLAKLKSTYTDKLPLMINPKTGRVHTSYHQAVTATGRLSSTDPNLQNIPVRNEEGRRIRQAFIAPEDYVIVSADYSQIELRIMAHLSRDKGLLTAFAEGKDIHRATAAEVFGLPLETVTSEQRRSAKAINFGLIYGMSAFGLARQLNIPRKEAQKYMDLYFERYPGVLEYMERTRAQAKEQGYVETLDGRRLYLPDIKSSNGARRAAAERAAINAPMQGTAADIIKRAMIAVDAWLQAEQPRVRMIMQVHDELVFEVHKDDVDAVAKQIHQLMENCTRLDVPLLVEVGSGENWDQAHSGGSKRTADGSEFESPKKKRKV

SEQ ID NO: 169 - protein, 2176 aa, synthetic construct
MRRRPRSGRYLRSIMAARRDSGARRWCFTLNNYTPEEEETARNLIHDADKYAFAIIGKEVGESGTPHLQGFMHFKQKQRLTALKKLFPRAHFEKARGSDQQNADYCGKDGEILTMIGTPSDNNPSGGSSGSETPGTSESATPESSGGSDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSKRTADGSEFESPKKKRKVSGGSSGGSVISYDNYVTILDEETLKAWIAKLEKAPVFAFATATDSLDNISANLVGLSFAIEPGVAAYIPVAHDYLDAPDQISRERALELLKPLLEDEKALKVGQNLKYDRGILANYGIELRGIAFDTMLESYILNSVAGRHDMDSLAERWLKHKTITFEEIAGKGKNQLTFNQIALEEAGRYAAEDADVTLQLHLKMWPDLQKHKGPLNVFENIEMPLVPVLSRIERNGVKIDPKVLHNHSEELTLRLAELEKKAHEIAGEEFNLSSTKQLQTILFEKQGIKPLKKTPGGAPSTSEEVLEELALDYPLPKVILEYRGLAKLKSTYTDKLPLMINPKTGRVHTSYHQAVTATGRLSSTDPNLQNIPVRNEEGRRIRQAFIAPEDYVIVSADYSQIELRIMAHLSRDKGLLTAFAEGKDIHRATAAEVFGLPLETVTSEQRRSAKAINFGLIYGMSAFGLARQLNIPRKEAQKYMDLYFERYPGVLEYMERTRAQAKEQGYVETLDGRRLYLPDIKSSNGARRAAAERAAINAPMQGTAADIIKRAMIAVDAWLQAEQPRVRMIMQVHDELVFEVHKDDVDAVAKQIHQLMENCTRLDVPLLVEVGSGENWDQAHSGGSKRTADGSEFESPKKKRKV

SEQ ID NO: 170 - protein, 2165 aa, synthetic construct
MPSKKSGPQPHKRWVFTLNNPSEEEKNKIRELPISLFDYFVCGEEGLEEGRTPHLQGFANFAKKQTFNKVKWYFGARCHIEKAKGTDQQNKEYCSKEGHILIECGAPRNQGKRSGGSSGSETPGTSESATPESSGGSDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSKRTADGSEFESPKKKRKVSGGSSGGSVISYDNYVTILDEETLKAWIAKLEKAPVFAFATATDSLDNISANLVGLSFAIEPGVAAYIPVAHDYLDAPDQISRERALELLKPLLEDEKALKVGQNLKYDRGILANYGIELRGIAFDTMLESYILNSVAGRHDMDSLAERWLKHKTITFEEIAGKGKNQLTFNQIALEEAGRYAAEDADVTLQLHLKMWPDLQKHKGPLNVFENIEMPLVPVLSRIERNGVKIDPKVLHNHSEELTLRLAELEKKAHEIAGEEFNLSSTKQLQTILFEKQGIKPLKKTPGGAPSTSEEVLEELALDYPLPKVILEYRGLAKLKSTYTDKLPLMINPKTGRVHTSYHQAVTATGRLSSTDPNLQNIPVRNEEGRRIRQAFIAPEDYVIVSADYSQIELRIMAHLSRDKGLLTAFAEGKDIHRATAAEVFGLPLETVTSEQRRSAKAINFGLIYGMSAFGLARQLNIPRKEAQKYMDLYFERYPGVLEYMERTRAQAKEQGYVETLDGRRLYLPDIKSSNGARRAAAERAAINAPMQGTAADIIKRAMIAVDAWLQAEQPRVRMIMQVHDELVFEVHKDDVDAVAKQIHQLMENCTRLDVPLLVEVGSGENWDQAHSGGSKRTADGSEFESPKKKRKV

SEQ ID NO: 171 - protein, 2159 aa, synthetic construct
MANSTVRRFVFTLNNYTEDQYQKCVEFISTKCKYGIVGKEVGEENGTPHLQGFCNLHKPMRFGTIKKSIDNAIHIEKANGSDIDNQKYCSKAGNFFETGTPSQQGRRSGGSSGSETPGTSESATPESSGGSDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSKRTADGSEFESPKKKRKVSGGSSGGSVISYDNYVTILDEETLKAWIAKLEKAPVFAFATATDSLDNISANLVGLSFAIEPGVAAYIPVAHDYLDAPDQISRERALELLKPLLEDEKALKVGQNLKYDRGILANYGIELRGIAFDTMLESYILNSVAGRHDMDSLAERWLKHKTITFEEIAGKGKNQLTFNQIALEEAGRYAAEDADVTLQLHLKMWPDLQKHKGPLNVFENIEMPLVPVLSRIERNGVKIDPKVLHNHSEELTLRLAELEKKAHEIAGEEFNLSSTKQLQTILFEKQGIKPLKKTPGGAPSTSEEVLEELALDYPLPKVILEYRGLAKLKSTYTDKLPLMINPKTGRVHTSYHQAVTATGRLSSTDPNLQNIPVRNEEGRRIRQAFIAPEDYVIVSADYSQIELRIMAHLSRDKGLLTAFAEGKDIHRATAAEVFGLPLETVTSEQRRSAKAINFGLIYGMSAFGLARQLNIPRKEAQKYMDLYFERYPGVLEYMERTRAQAKEQGYVETLDGRRLYLPDIKSSNGARRAAAERAAINAPMQGTAADIIKRAMIAVDAWLQAEQPRVRMIMQVHDELVFEVHKDDVDAVAKQIHQLMENCTRLDVPLLVEVGSGENWDQAHSGGSKRTADGSEFESPKKKRKV

SEQ ID NO: 172 - protein, 2167 aa, synthetic construct
MALADVRGMANSTVRRFCFTWNNYSELEYVLCCEFIKKYCCYGIVGKEIAPNTGTHHLQGFCNLEKPMRFSTIKKRLDSRIHIEKAAGSDSENQTYCSKTGNFFESGTPNQQGRRSGGSSGSETPGTSESATPESSGGSDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSKRTADGSEFESPKKKRKVSGGSSGGSVISYDNYVTILDEETLKAWIAKLEKAPVFAFATATDSLDNISANLVGLSFAIEPGVAAYIPVAHDYLDAPDQISRERALELLKPLLEDEKALKVGQNLKYDRGILANYGIELRGIAFDTMLESYILNSVAGRHDMDSLAERWLKHKTITFEEIAGKGKNQLTFNQIALEEAGRYAAEDADVTLQLHLKMWPDLQKHKGPLNVFENIEMPLVPVLSRIERNGVKIDPKVLHNHSEELTLRLAELEKKAHEIAGEEFNLSSTKQLQTILFEKQGIKPLKKTPGGAPSTSEEVLEELALDYPLPKVILEYRGLAKLKSTYTDKLPLMINPKTGRVHTSYHQAVTATGRLSSTDPNLQNIPVRNEEGRRIRQAFIAPEDYVIVSADYSQIELRIMAHLSRDKGLLTAFAEGKDIHRATAAEVFGLPLETVTSEQRRSAKAINFGLIYGMSAFGLARQLNIPRKEAQKYMDLYFERYPGVLEYMERTRAQAKEQGYVETLDGRRLYLPDIKSSNGARRAAAERAAINAPMQGTAADIIKRAMIAVDAWLQAEQPRVRMIMQVHDELVFEVHKDDVDAVAKQIHQLMENCTRLDVPLLVEVGSGENWDQAHSGGSKRTADGSEFESPKKKRKV

SEQ ID NO: 173 - protein, 2159 aa, synthetic construct
MANRTVRRFCFTWNDHPVEAYEKCEKFIEKFCKYGIVGEEYAPTTGMPHLQGFCNLNKPTRFSTIKKHLDNSIHIEKANGTDEQNQKYCSKSGIFFESGVPNKQGQRSGGSSGSETPGTSESATPESSGGSDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSKRTADGSEFESPKKKRKVSGGSSGGSVISYDNYVTILDEETLKAWIAKLEKAPVFAFATATDSLDNISANLVGLSFAIEPGVAAYIPVAHDYLDAPDQISRERALELLKPLLEDEKALKVGQNLKYDRGILANYGIELRGIAFDTMLESYILNSVAGRHDMDSLAERWLKHKTITFEEIAGKGKNQLTFNQIALEEAGRYAAEDADVTLQLHLKMWPDLQKHKGPLNVFENIEMPLVPVLSRIERNGVKIDPKVLHNHSEELTLRLAELEKKAHEIAGEEFNLSSTKQLQTILFEKQGIKPLKKTPGGAPSTSEEVLEELALDYPLPKVILEYRGLAKLKSTYTDKLPLMINPKTGRVHTSYHQAVTATGRLSSTDPNLQNIPVRNEEGRRIRQAFIAPEDYVIVSADYSQIELRIMAHLSRDKGLLTAFAEGKDIHRATAAEVFGLPLETVTSEQRRSAKAINFGLIYGMSAFGLARQLNIPRKEAQKYMDLYFERYPGVLEYMERTRAQAKEQGYVETLDGRRLYLPDIKSSNGARRAAAERAAINAPMQGTAADIIKRAMIAVDAWLQAEQPRVRMIMQVHDELVFEVHKDDVDAVAKQIHQLMENCTRLDVPLLVEVGSGENWDQAHSGGSKRTADGSEFESPKKKRKV

SEQ ID NO: 174 - protein, 2158 aa, synthetic construct
MNSTVRRFCFTWNNYTVADTLTVKDYFVKYCKYGIAGEELAPETGTPHLQGFCNLRKPQRFSAIKKHLSDRIHIEKANGSDEQNQSYCKKAGHWFEQGVPVKQGDRSGGSSGSETPGTSESATPESSGGSDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSKRTADGSEFESPKKKRKVSGGSSGGSVISYDNYVTILDEETLKAWIAKLEKAPVFAFATATDSLDNISANLVGLSFAIEPGVAAYIPVAHDYLDAPDQISRERALELLKPLLEDEKALKVGQNLKYDRGILANYGIELRGIAFDTMLESYILNSVAGRHDMDSLAERWLKHKTITFEEIAGKGKNQLTFNQIALEEAGRYAAEDADVTLQLHLKMWPDLQKHKGPLNVFENIEMPLVPVLSRIERNGVKIDPKVLHNHSEELTLRLAELEKKAHEIAGEEFNLSSTKQLQTILFEKQGIKPLKKTPGGAPSTSEEVLEELALDYPLPKVILEYRGLAKLKSTYTDKLPLMINPKTGRVHTSYHQAVTATGRLSSTDPNLQNIPVRNEEGRRIRQAFIAPEDYVIVSADYSQIELRIMAHLSRDKGLLTAFAEGKDIHRATAAEVFGLPLETVTSEQRRSAKAINFGLIYGMSAFGLARQLNIPRKEAQKYMDLYFERYPGVLEYMERTRAQAKEQGYVETLDGRRLYLPDIKSSNGARRAAAERAAINAPMQGTAADIIKRAMIAVDAWLQAEQPRVRMIMQVHDELVFEVHKDDVDAVAKQIHQLMENCTRLDVPLLVEVGSGENWDQAHSGGSKRTADGSEFESPKKKRKV

SEQ ID NO: 175 - protein, 2159 aa, synthetic construct
MANRTVRRFCFTWNNYPDTAYEKCETFIQKFCKYGIVGEELAPTTGTPHLQGFCNLRKPTRFSTIKKHLDNSIHIEKANGSDEQNQAYCSKTGIFFEEGSPTKQGQRSGGSSGSETPGTSESATPESSGGSDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSKRTADGSEFESPKKKRKVSGGSSGGSVISYDNYVTILDEETLKAWIAKLEKAPVFAFATATDSLDNISANLVGLSFAIEPGVAAYIPVAHDYLDAPDQISRERALELLKPLLEDEKALKVGQNLKYDRGILANYGIELRGIAFDTMLESYILNSVAGRHDMDSLAERWLKHKTITFEEIAGKGKNQLTFNQIALEEAGRYAAEDADVTLQLHLKMWPDLQKHKGPLNVFENIEMPLVPVLSRIERNGVKIDPKVLHNHSEELTLRLAELEKKAHEIAGEEFNLSSTKQLQTILFEKQGIKPLKKTPGGAPSTSEEVLEELALDYPLPKVILEYRGLAKLKSTYTDKLPLMINPKTGRVHTSYHQAVTATGRLSSTDPNLQNIPVRNEEGRRIRQAFIAPEDYVIVSADYSQIELRIMAHLSRDKGLLTAFAEGKDIHRATAAEVFGLPLETVTSEQRRSAKAINFGLIYGMSAFGLARQLNIPRKEAQKYMDLYFERYPGVLEYMERTRAQAKEQGYVETLDGRRLYLPDIKSSNGARRAAAERAAINAPMQGTAADIIKRAMIAVDAWLQAEQPRVRMIMQVHDELVFEVHKDDVDAVAKQIHQLMENCTRLDVPLLVEVGSGENWDQAHSGGSKRTADGSEFESPKKKRKV

SEQ ID NO: 176 - protein, 2159 aa, synthetic construct
MSNSTVRRFCFTWNNYTELNYALCQEFIKKYCKYGIVGKELAPTTNTPHLQGFCNLQKPMRFSTIKKRLDNGIHIEKSMGSDTQNQTYCSKSGEFFEAGDPQCQGKRSGGSSGSETPGTSESATPESSGGSDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSKRTADGSEFESPKKKRKVSGGSSGGSVISYDNYVTILDEETLKAWIAKLEKAPVFAFATATDSLDNISANLVGLSFAIEPGVAAYIPVAHDYLDAPDQISRERALELLKPLLEDEKALKVGQNLKYDRGILANYGIELRGIAFDTMLESYILNSVAGRHDMDSLAERWLKHKTITFEEIAGKGKNQLTFNQIALEEAGRYAAEDADVTLQLHLKMWPDLQKHKGPLNVFENIEMPLVPVLSRIERNGVKIDPKVLHNHSEELTLRLAELEKKAHEIAGEEFNLSSTKQLQTILFEKQGIKPLKKTPGGAPSTSEEVLEELALDYPLPKVILEYRGLAKLKSTYTDKLPLMINPKTGRVHTSYHQAVTATGRLSSTDPNLQNIPVRNEEGRRIRQAFIAPEDYVIVSADYSQIELRIMAHLSRDKGLLTAFAEGKDIHRATAAEVFGLPLETVTSEQRRSAKAINFGLIYGMSAFGLARQLNIPRKEAQKYMDLYFERYPGVLEYMERTRAQAKEQGYVETLDGRRLYLPDIKSSNGARRAAAERAAINAPMQGTAADIIKRAMIAVDAWLQAEQPRVRMIMQVHDELVFEVHKDDVDAVAKQIHQLMENCTRLDVPLLVEVGSGENWDQAHSGGSKRTADGSEFESPKKKRKV

SEQ ID NO: 177 - protein, 2166 aa, synthetic construct
MAVSSRKQTNSTLRRFCWTLNNYTEEDVTTLQKDLAELCKFAIFGRETCPNTGTKHLQGFCNLQRPKRFSSIRKLFKERAHIEKAKGSDFDNKAYCSKSGEVWMHGEPSSQGARSGGSSGSETPGTSESATPESSGGSDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSKRTADGSEFESPKKKRKVSGGSSGGSVISYDNYVTILDEETLKAWIAKLEKAPVFAFATATDSLDNISANLVGLSFAIEPGVAAYIPVAHDYLDAPDQISRERALELLKPLLEDEKALKVGQNLKYDRGILANYGIELRGIAFDTMLESYILNSVAGRHDMDSLAERWLKHKTITFEEIAGKGKNQLTFNQIALEEAGRYAAEDADVTLQLHLKMWPDLQKHKGPLNVFENIEMPLVPVLSRIERNGVKIDPKVLHNHSEELTLRLAELEKKAHEIAGEEFNLSSTKQLQTILFEKQGIKPLKKTPGGAPSTSEEVLEELALDYPLPKVILEYRGLAKLKSTYTDKLPLMINPKTGRVHTSYHQAVTATGRLSSTDPNLQNIPVRNEEGRRIRQAFIAPEDYVIVSADYSQIELRIMAHLSRDKGLLTAFAEGKDIHRATAAEVFGLPLETVTSEQRRSAKAINFGLIYGMSAFGLARQLNIPRKEAQKYMDLYFERYPGVLEYMERTRAQAKEQGYVETLDGRRLYLPDIKSSNGARRAAAERAAINAPMQGTAADIIKRAMIAVDAWLQAEQPRVRMIMQVHDELVFEVHKDDVDAVAKQIHQLMENCTRLDVPLLVEVGSGENWDQAHSGGSKRTADGSEFESPKKKRKV

SEQ ID NO: 178 - protein, 2159 aa, synthetic construct
MSNRTVRKFCFTWNNYEFDAYAKCETFLNNFAKYGIVGEELCPSTGTPHLQGYVNLIKPTRFSTIKKHLHNAIHIEKANGSDEQNQTYCRKSGIFFEKGEPIKQGQRSGGSSGSETPGTSESATPESSGGSDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSKRTADGSEFESPKKKRKVSGGSSGGSVISYDNYVTILDEETLKAWIAKLEKAPVFAFATATDSLDNISANLVGLSFAIEPGVAAYIPVAHDYLDAPDQISRERALELLKPLLEDEKALKVGQNLKYDRGILANYGIELRGIAFDTMLESYILNSVAGRHDMDSLAERWLKHKTITFEEIAGKGKNQLTFNQIALEEAGRYAAEDADVTLQLHLKMWPDLQKHKGPLNVFENIEMPLVPVLSRIERNGVKIDPKVLHNHSEELTLRLAELEKKAHEIAGEEFNLSSTKQLQTILFEKQGIKPLKKTPGGAPSTSEEVLEELALDYPLPKVILEYRGLAKLKSTYTDKLPLMINPKTGRVHTSYHQAVTATGRLSSTDPNLQNIPVRNEEGRRIRQAFIAPEDYVIVSADYSQIELRIMAHLSRDKGLLTAFAEGKDIHRATAAEVFGLPLETVTSEQRRSAKAINFGLIYGMSAFGLARQLNIPRKEAQKYMDLYFERYPGVLEYMERTRAQAKEQGYVETLDGRRLYLPDIKSSNGARRAAAERAAINAPMQGTAADIIKRAMIAVDAWLQAEQPRVRMIMQVHDELVFEVHKDDVDAVAKQIHQLMENCTRLDVPLLVEVGSGENWDQAHSGGSKRTADGSEFESPKKKRKV

SEQ ID NO: 179 - protein, 2159 aa, synthetic construct
MAVRGSAAKRWCFTLNNPTEEEIAAVKAWQHSEYHYAIVGKEKGEQGTPHLQGFIHLKKKVRLTSLKKVLQRAHWEKARGSDEDNEKYCSKEGDVILTIGIPVKGNRSGGSSGSETPGTSESATPESSGGSDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSKRTADGSEFESPKKKRKVSGGSSGGSVISYDNYVTILDEETLKAWIAKLEKAPVFAFATATDSLDNISANLVGLSFAIEPGVAAYIPVAHDYLDAPDQISRERALELLKPLLEDEKALKVGQNLKYDRGILANYGIELRGIAFDTMLESYILNSVAGRHDMDSLAERWLKHKTITFEEIAGKGKNQLTFNQIALEEAGRYAAEDADVTLQLHLKMWPDLQKHKGPLNVFENIEMPLVPVLSRIERNGVKIDPKVLHNHSEELTLRLAELEKKAHEIAGEEFNLSSTKQLQTILFEKQGIKPLKKTPGGAPSTSEEVLEELALDYPLPKVILEYRGLAKLKSTYTDKLPLMINPKTGRVHTSYHQAVTATGRLSSTDPNLQNIPVRNEEGRRIRQAFIAPEDYVIVSADYSQIELRIMAHLSRDKGLLTAFAEGKDIHRATAAEVFGLPLETVTSEQRRSAKAINFGLIYGMSAFGLARQLNIPRKEAQKYMDLYFERYPGVLEYMERTRAQAKEQGYVETLDGRRLYLPDIKSSNGARRAAAERAAINAPMQGTAADIIKRAMIAVDAWLQAEQPRVRMIMQVHDELVFEVHKDDVDAVAKQIHQLMENCTRLDVPLLVEVGSGENWDQAHSGGSKRTADGSEFESPKKKRKV

SEQ ID NO: 180 - protein, 2160 aa, synthetic construct
MAPVRAAAAKRWCFTLNNYTAEEEAKVRALLPGEFHFAICGKERGEQGTPHLQGFLHFKKKQRLSALKKLLARAHWEKARGSDHDNEEYCSKENDVILTIGEPVQGNRSGGSSGSETPGTSESATPESSGGSDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSKRTADGSEFESPKKKRKVSGGSSGGSVISYDNYVTILDEETLKAWIAKLEKAPVFAFATATDSLDNISANLVGLSFAIEPGVAAYIPVAHDYLDAPDQISRERALELLKPLLEDEKALKVGQNLKYDRGILANYGIELRGIAFDTMLESYILNSVAGRHDMDSLAERWLKHKTITFEEIAGKGKNQLTFNQIALEEAGRYAAEDADVTLQLHLKMWPDLQKHKGPLNVFENIEMPLVPVLSRIERNGVKIDPKVLHNHSEELTLRLAELEKKAHEIAGEEFNLSSTKQLQTILFEKQGIKPLKKTPGGAPSTSEEVLEELALDYPLPKVILEYRGLAKLKSTYTDKLPLMINPKTGRVHTSYHQAVTATGRLSSTDPNLQNIPVRNEEGRRIRQAFIAPEDYVIVSADYSQIELRIMAHLSRDKGLLTAFAEGKDIHRATAAEVFGLPLETVTSEQRRSAKAINFGLIYGMSAFGLARQLNIPRKEAQKYMDLYFERYPGVLEYMERTRAQAKEQGYVETLDGRRLYLPDIKSSNGARRAAAERAAINAPMQGTAADIIKRAMIAVDAWLQAEQPRVRMIMQVHDELVFEVHKDDVDAVAKQIHQLMENCTRLDVPLLVEVGSGENWDQAHSGGSKRTADGSEFESPKKKRKV

SEQ ID NO: 181 - protein, 2161 aa, synthetic construct
MAPIKRPAPCKRWCFTLNNFTDDEVQKVVSLQPDEVHYAIVGRETGAQGTPHLQGYLHLKKKKRLTSMKEFLPRAHWEVARGSDEDNEAYCSKEGDVILTLGIPAKGNRSGGSSGSETPGTSESATPESSGGSDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSKRTADGSEFESPKKKRKVSGGSSGGSVISYDNYVTILDEETLKAWIAKLEKAPVFAFATATDSLDNISANLVGLSFAIEPGVAAYIPVAHDYLDAPDQISRERALELLKPLLEDEKALKVGQNLKYDRGILANYGIELRGIAFDTMLESYILNSVAGRHDMDSLAERWLKHKTITFEEIAGKGKNQLTFNQIALEEAGRYAAEDADVTLQLHLKMWPDLQKHKGPLNVFENIEMPLVPVLSRIERNGVKIDPKVLHNHSEELTLRLAELEKKAHEIAGEEFNLSSTKQLQTILFEKQGIKPLKKTPGGAPSTSEEVLEELALDYPLPKVILEYRGLAKLKSTYTDKLPLMINPKTGRVHTSYHQAVTATGRLSSTDPNLQNIPVRNEEGRRIRQAFIAPEDYVIVSADYSQIELRIMAHLSRDKGLLTAFAEGKDIHRATAAEVFGLPLETVTSEQRRSAKAINFGLIYGMSAFGLARQLNIPRKEAQKYMDLYFERYPGVLEYMERTRAQAKEQGYVETLDGRRLYLPDIKSSNGARRAAAERAAINAPMQGTAADIIKRAMIAVDAWLQAEQPRVRMIMQVHDELVFEVHKDDVDAVAKQIHQLMENCTRLDVPLLVEVGSGENWDQAHSGGSKRTADGSEFESPKKKRKV

SEQ ID NO: 182 - protein, 2163 aa, synthetic construct
MAKNGNYSYKRWVFTINNPTFEDYCAVVEFCNLDNCKFAIVGEEKGEKEGTPHLQGFLSLRKNAKAAALEENLGGRAWLSRARGSDEDNEEYCSKESTYLRVGEPNRKGRSSGGSSGSETPGTSESATPESSGGSDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSKRTADGSEFESPKKKRKVSGGSSGGSVISYDNYVTILDEETLKAWIAKLEKAPVFAFATATDSLDNISANLVGLSFAIEPGVAAYIPVAHDYLDAPDQISRERALELLKPLLEDEKALKVGQNLKYDRGILANYGIELRGIAFDTMLESYILNSVAGRHDMDSLAERWLKHKTITFEEIAGKGKNQLTFNQIALEEAGRYAAEDADVTLQLHLKMWPDLQKHKGPLNVFENIEMPLVPVLSRIERNGVKIDPKVLHNHSEELTLRLAELEKKAHEIAGEEFNLSSTKQLQTILFEKQGIKPLKKTPGGAPSTSEEVLEELALDYPLPKVILEYRGLAKLKSTYTDKLPLMINPKTGRVHTSYHQAVTATGRLSSTDPNLQNIPVRNEEGRRIRQAFIAPEDYVIVSADYSQIELRIMAHLSRDKGLLTAFAEGKDIHRATAAEVFGLPLETVTSEQRRSAKAINFGLIYGMSAFGLARQLNIPRKEAQKYMDLYFERYPGVLEYMERTRAQAKEQGYVETLDGRRLYLPDIKSSNGARRAAAERAAINAPMQGTAADIIKRAMIAVDAWLQAEQPRVRMIMQVHDELVFEVHKDDVDAVAKQIHQLMENCTRLDVPLLVEVGSGENWDQAHSGGSKRTADGSEFESPKKKRKV

SEQ ID NO: 183 - protein, 2163 aa, synthetic construct
MAKKSDYGYKRWVFTINNPTFEDYVSVIEFCTAENCKFAIVGEEKGEKEGTPHLQGFLSLRKNARAAALEENLGGRAWLSRAVGSDEENEEYCSKETTYLRVGTPNRKGRSSGGSSGSETPGTSESATPESSGGSDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSKRTADGSEFESPKKKRKVSGGSSGGSVISYDNYVTILDEETLKAWIAKLEKAPVFAFATATDSLDNISANLVGLSFAIEPGVAAYIPVAHDYLDAPDQISRERALELLKPLLEDEKALKVGQNLKYDRGILANYGIELRGIAFDTMLESYILNSVAGRHDMDSLAERWLKHKTITFEEIAGKGKNQLTFNQIALEEAGRYAAEDADVTLQLHLKMWPDLQKHKGPLNVFENIEMPLVPVLSRIERNGVKIDPKVLHNHSEELTLRLAELEKKAHEIAGEEFNLSSTKQLQTILFEKQGIKPLKKTPGGAPSTSEEVLEELALDYPLPKVILEYRGLAKLKSTYTDKLPLMINPKTGRVHTSYHQAVTATGRLSSTDPNLQNIPVRNEEGRRIRQAFIAPEDYVIVSADYSQIELRIMAHLSRDKGLLTAFAEGKDIHRATAAEVFGLPLETVTSEQRRSAKAINFGLIYGMSAFGLARQLNIPRKEAQKYMDLYFERYPGVLEYMERTRAQAKEQGYVETLDGRRLYLPDIKSSNGARRAAAERAAINAPMQGTAADIIKRAMIAVDAWLQAEQPRVRMIMQVHDELVFEVHKDDVDAVAKQIHQLMENCTRLDVPLLVEVGSGENWDQAHSGGSKRTADGSEFESPKKKRKV

SEQ ID NO: 184 - protein, 2166 aa, synthetic construct
MESKRKKRQNPAKRWCFTLNNYTQLEAITIEQLLCTEEVQYAIVGEEIGENGTPHLQGFFNLKKKKRLTSLKAWLNDRAHYEEAKGSDEQNRRYCSKSGNILISFGSPQKQGQRSGGSSGSETPGTSESATPESSGGSDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSKRTADGSEFESPKKKRKVSGGSSGGSVISYDNYVTILDEETLKAWIAKLEKAPVFAFATATDSLDNISANLVGLSFAIEPGVAAYIPVAHDYLDAPDQISRERALELLKPLLEDEKALKVGQNLKYDRGILANYGIELRGIAFDTMLESYILNSVAGRHDMDSLAERWLKHKTITFEEIAGKGKNQLTFNQIALEEAGRYAAEDADVTLQLHLKMWPDLQKHKGPLNVFENIEMPLVPVLSRIERNGVKIDPKVLHNHSEELTLRLAELEKKAHEIAGEEFNLSSTKQLQTILFEKQGIKPLKKTPGGAPSTSEEVLEELALDYPLPKVILEYRGLAKLKSTYTDKLPLMINPKTGRVHTSYHQAVTATGRLSSTDPNLQNIPVRNEEGRRIRQAFIAPEDYVIVSADYSQIELRIMAHLSRDKGLLTAFAEGKDIHRATAAEVFGLPLETVTSEQRRSAKAINFGLIYGMSAFGLARQLNIPRKEAQKYMDLYFERYPGVLEYMERTRAQAKEQGYVETLDGRRLYLPDIKSSNGARRAAAERAAINAPMQGTAADIIKRAMIAVDAWLQAEQPRVRMIMQVHDELVFEVHKDDVDAVAKQIHQLMENCTRLDVPLLVEVGSGENWDQAHSGGSKRTADGSEFESPKKKRKV

SEQ ID NO: 185 - protein, 2161 aa, synthetic construct
MPPQKREAAAKRWCFTLNNYTDEEVSAVKAWNASEYHYAVVGREKGENGTPHLQGYIHLKKKARLSTLKKLLSRAHWEKARGSDSDNEAYCTKDGDVILTLGMPVEGNRSGGSSGSETPGTSESATPESSGGSDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSKRTADGSEFESPKKKRKVSGGSSGGSVISYDNYVTILDEETLKAWIAKLEKAPVFAFATATDSLDNISANLVGLSFAIEPGVAAYIPVAHDYLDAPDQISRERALELLKPLLEDEKALKVGQNLKYDRGILANYGIELRGIAFDTMLESYILNSVAGRHDMDSLAERWLKHKTITFEEIAGKGKNQLTFNQIALEEAGRYAAEDADVTLQLHLKMWPDLQKHKGPLNVFENIEMPLVPVLSRIERNGVKIDPKVLHNHSEELTLRLAELEKKAHEIAGEEFNLSSTKQLQTILFEKQGIKPLKKTPGGAPSTSEEVLEELALDYPLPKVILEYRGLAKLKSTYTDKLPLMINPKTGRVHTSYHQAVTATGRLSSTDPNLQNIPVRNEEGRRIRQAFIAPEDYVIVSADYSQIELRIMAHLSRDKGLLTAFAEGKDIHRATAAEVFGLPLETVTSEQRRSAKAINFGLIYGMSAFGLARQLNIPRKEAQKYMDLYFERYPGVLEYMERTRAQAKEQGYVETLDGRRLYLPDIKSSNGARRAAAERAAINAPMQGTAADIIKRAMIAVDAWLQAEQPRVRMIMQVHDELVFEVHKDDVDAVAKQIHQLMENCTRLDVPLLVEVGSGENWDQAHSGGSKRTADGSEFESPKKKRKV

SEQ ID NO: 186 - protein, 2251 aa, synthetic construct
MRMSKMARKDPTIEFCQLGLDTFETKYITMFGMLVSCSFDKPAFISFVFSDFTKNDIVQNYLYDRYLIDYENKLELNEGFKAIMYKNQFETFDSKLRKIFNNGLRDLQNGRDENLSQYGIVCKMNIKVKMYNGKLNAIVRECEPVPHSQISSIASPSQCEHLRLFYQRAFKRIGESAISRYFEEYRRFFPIHRNGSHLASGGSSGSETPGTSESATPESSGGSDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSKRTADGSEFESPKKKRKVSGGSSGGSVISYDNYVTILDEETLKAWIAKLEKAPVFAFATATDSLDNISANLVGLSFAIEPGVAAYIPVAHDYLDAPDQISRERALELLKPLLEDEKALKVGQNLKYDRGILANYGIELRGIAFDTMLESYILNSVAGRHDMDSLAERWLKHKTITFEEIAGKGKNQLTFNQIALEEAGRYAAEDADVTLQLHLKMWPDLQKHKGPLNVFENIEMPLVPVLSRIERNGVKIDPKVLHNHSEELTLRLAELEKKAHEIAGEEFNLSSTKQLQTILFEKQGIKPLKKTPGGAPSTSEEVLEELALDYPLPKVILEYRGLAKLKSTYTDKLPLMINPKTGRVHTSYHQAVTATGRLSSTDPNLQNIPVRNEEGRRIRQAFIAPEDYVIVSADYSQIELRIMAHLSRDKGLLTAFAEGKDIHRATAAEVFGLPLETVTSEQRRSAKAINFGLIYGMSAFGLARQLNIPRKEAQKYMDLYFERYPGVLEYMERTRAQAKEQGYVETLDGRRLYLPDIKSSNGARRAAAERAAINAPMQGTAADIIKRAMIAVDAWLQAEQPRVRMIMQVHDELVFEVHKDDVDAVAKQIHQLMENCTRLDVPLLVEVGSGENWDQAHSGGSKRTADGSEFESPKKKRKV

SEQ ID NO: 187 - protein, 2251 aa, synthetic construct
MRMSKMARKDPTIEFCQLGLDTFETKYITMFGMLVSCSFDKPAFISFVFSDFTKNDIVQNALYDRYLIDYENKLELNEGFKAIMYKNQFETFDSKLRKIFNNGLRDLQNGRDENLSQYGIVCKMNIKVKMYNGKLNAIVRECEPVPHSQISSIASPSQCEHLRLFYQRAFKRIGESAISRYFEEYRRFFPIHRNGSHLASGGSSGSETPGTSESATPESSGGSDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSKRTADGSEFESPKKKRKVSGGSSGGSVISYDNYVTILDEETLKAWIAKLEKAPVFAFATATDSLDNISANLVGLSFAIEPGVAAYIPVAHDYLDAPDQISRERALELLKPLLEDEKALKVGQNLKYDRGILANYGIELRGIAFDTMLESYILNSVAGRHDMDSLAERWLKHKTITFEEIAGKGKNQLTFNQIALEEAGRYAAEDADVTLQLHLKMWPDLQKHKGPLNVFENIEMPLVPVLSRIERNGVKIDPKVLHNHSEELTLRLAELEKKAHEIAGEEFNLSSTKQLQTILFEKQGIKPLKKTPGGAPSTSEEVLEELALDYPLPKVILEYRGLAKLKSTYTDKLPLMINPKTGRVHTSYHQAVTATGRLSSTDPNLQNIPVRNEEGRRIRQAFIAPEDYVIVSADYSQIELRIMAHLSRDKGLLTAFAEGKDIHRATAAEVFGLPLETVTSEQRRSAKAINFGLIYGMSAFGLARQLNIPRKEAQKYMDLYFERYPGVLEYMERTRAQAKEQGYVETLDGRRLYLPDIKSSNGARRAAAERAAINAPMQGTAADIIKRAMIAVDAWLQAEQPRVRMIMQVHDELVFEVHKDDVDAVAKQIHQLMENCTRLDVPLLVEVGSGENWDQAHSGGSKRTADGSEFESPKKKRKV

SEQ ID NO: 188 - protein, 2389 aa, synthetic construct
MSPSKKNGRSGPQPHKRWVFTLNNPSEDERKKIRDLPISLFDYFIVGEEGNEEGRTPHLQGFANFVKKQTFNKVKWYLGARCHIEKAKGTDQQNKEYCSKEGNLLMECGAPRSQGQRSGGSSGSETPGTSESATPESSGGSDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSKRTADGSEFESPKKKRKVSGGSSGGSVISYDNYVTILDEETLKAWIAKLEKAPVFAFATATDSLDNISANLVGLSFAIEPGVAAYIPVAHDYLDAPDQISRERALELLKPLLEDEKALKVGQNLKYDRGILANYGIELRGIAFDTMLESYILNSVAGRHDMDSLAERWLKHKTITFEEIAGKGKNQLTFNQIALEEAGRYAAEDADVTLQLHLKMWPDLQKHKGPLNVFENIEMPLVPVLSRIERNGVKIDPKVLHNHSEELTLRLAELEKKAHEIAGEEFNLSSTKQLQTILFEKQGIKPLKKTPGGAPSTSEEVLEELALDYPLPKVILEYRGLAKLKSTYTDKLPLMINPKTGRVHTSYHQAVTATGRLSSTDPNLQNIPVRNEEGRRIRQAFIAPEDYVIVSADYSQIELRIMAHLSRDKGLLTAFAEGKDIHRATAAEVFGLPLETVTSEQRRSAKAINFGLIYGMSAFGLARQLNIPRKEAQKYMDLYFERYPGVLEYMERTRAQAKEQGYVETLDGRRLYLPDIKSSNGARRAAAERAAINAPMQGTAADIIKRAMIAVDAWLQAEQPRVRMIMQVHDELVFEVHKDDVDAVAKQIHQLMENCTRLDVPLLVEVGSGENWDQAHSGGSKRTADGSEFESPKKKRKVGSGATNFSLLKQAGDVEENPGPTEYKPTVRLATRDDVPRAVRTLAAAFADYPATRHTVDPDRHIERVTELQELFLTRVGLDIGKVWVADDGAAVAVWTTPESVEAGAVFAEIGPRMAELSGSRLAAQQQMEGLLAPHRPKEPAWFLATVGVSPDHQGKGLGSAVVLPGVEAAERAGVPAFLETSAPRNLPFYERLGFTVTADVEVPEGPRTWCMTRKPGA

SEQ ID NO: 189 - protein, 2239 aa, synthetic construct
MSPSKKNGRSGPQPHKRWVFTLNNPSEDERKKIRDLPISLFDYFIVGEEGNEEGRTPHLQGFANFVKKQTFNKVKWYLGARCHIEKAKGTDQQNKEYCSKEGNLLMECGAPRSQGQRSGGSSGSETPGTSESATPESSGGSDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSSGSETPGTSESATPESSGGSSGGSSTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGLLTSEGKEIKNKDEILALLKALFLPKRLSIIHCPGHQKGHSAEARGNRMADQAARKAAITETPDTSTLLIENSSPSGGSKRTADGSEFEPKKKRKV

SEQ ID NO: 190 - protein, 1855 aa, synthetic construct
MSPSKKNGRSGPQPHKRWVFTLNNPSEDERKKIRDLPISLFDYFIVGEEGNEEGRTPHLQGFANFVKKQTFNKVKWYLGARCHIEKAKGTDQQNKEYCSKEGNLLMECGAPRSQGQRSGGSSGSETPGTSESATPESSGGSGKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEEASKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKGSGGSSGGSKRTADGSEFESPKKKRKVSGGSSGGSVISYDNYVTILDEETLKAWIAKLEKAPVFAFATATDSLDNISANLVGLSFAIEPGVAAYIPVAHDYLDAPDQISRERALELLKPLLEDEKALKVGQNLKYDRGILANYGIELRGIAFDTMLESYILNSVAGRHDMDSLAERWLKHKTITFEEIAGKGKNQLTFNQIALEEAGRYAAEDADVTLQLHLKMWPDLQKHKGPLNVFENIEMPLVPVLSRIERNGVKIDPKVLHNHSEELTLRLAELEKKAHEIAGEEFNLSSTKQLQTILFEKQGIKPLKKTPGGAPSTSEEVLEELALDYPLPKVILEYRGLAKLKSTYTDKLPLMINPKTGRVHTSYHQAVTATGRLSSTDPNLQNIPVRNEEGRRIRQAFIAPEDYVIVSADYSQIELRIMAHLSRDKGLLTAFAEGKDIHRATAAEVFGLPLETVTSEQRRSAKAINFGLIYGMSAFGLARQLNIPRKEAQKYMDLYFERYPGVLEYMERTRAQAKEQGYVETLDGRRLYLPDIKSSNGARRAAAERAAINAPMQGTAADIIKRAMIAVDAWLQAEQPRVRMIMQVHDELVFEVHKDDVDAVAKQIHQLMENCTRLDVPLLVEVGSGENWDQAHSGGSKRTADGSEFESPKKKRKV

SEQ ID NO: 191 - protein, 2188 aa, synthetic construct
MGAQPARSMAEAGITGTWYNQSGSTFTVTAGADGNLTGQYENRAQGTGCQNSPYTLTGRYNGTKLEWRVEWNNSTENCHSRTEWRGQYQGGAEARINTQWNLTYEGGSGPATEQGQDTFTKVKPSAASGSSAGGGGSGGGGSGGGGSGPKKKRKVAAAGSDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSKRTADGSEFESPKKKRKVSGGSSGGSVISYDNYVTILDEETLKAWIAKLEKAPVFAFATATDSLDNISANLVGLSFAIEPGVAAYIPVAHDYLDAPDQISRERALELLKPLLEDEKALKVGQNLKYDRGILANYGIELRGIAFDTMLESYILNSVAGRHDMDSLAERWLKHKTITFEEIAGKGKNQLTFNQIALEEAGRYAAEDADVTLQLHLKMWPDLQKHKGPLNVFENIEMPLVPVLSRIERNGVKIDPKVLHNHSEELTLRLAELEKKAHEIAGEEFNLSSTKQLQTILFEKQGIKPLKKTPGGAPSTSEEVLEELALDYPLPKVILEYRGLAKLKSTYTDKLPLMINPKTGRVHTSYHQAVTATGRLSSTDPNLQNIPVRNEEGRRIRQAFIAPEDYVIVSADYSQIELRIMAHLSRDKGLLTAFAEGKDIHRATAAEVFGLPLETVTSEQRRSAKAINFGLIYGMSAFGLARQLNIPRKEAQKYMDLYFERYPGVLEYMERTRAQAKEQGYVETLDGRRLYLPDIKSSNGARRAAAERAAINAPMQGTAADIIKRAMIAVDAWLQAEQPRVRMIMQVHDELVFEVHKDDVDAVAKQIHQLMENCTRLDVPLLVEVGSGENWDQAHSGGSKRTADGSEFESPKKKRKV

SEQ ID NO: 192 - protein, 2149 aa, synthetic construct
MAEAGITGTWYNQSGSTFTVTAGADGNLTGQYENRAQGTGCQNSPYTLTGRYNGTKLEWRVEWNNSTENCHSRTEWRGQYQGGAEARINTQWNLTYEGGSGPATEQGQDTFTKVKPSAASGSSAGGGGSGGGGSGGGGSGPKKKRKVAAAGSDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSKRTADGSEFESPKKKRKVSGGSSGGSKHMPRKRYSCDFETTTKVEDCRVWAYGYMNIEDHSEYKIGNSLDEFMAWALKVQADLYFHNLKFDGAFIINWLERNGFKWSADGLPNTYNTIISRTGQWYMIDICLGYKGKRKIHTVIYDSLKKLPFPVKKIAKDFKLTVLKGDIDYHKERPVGYKITPEEYAYIKNAIQIIAEALLIQFKQGLDRMTAGSDSLKDFKDIITTKKFKKVFPTLSLGLDKKVRYAYRGGFTWLNDRFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSEGAIKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRPKTYIQDIYMKEVDGELVEGSPDDYTDIKLSVKCAGMTDKIKKEVTFENFKVGFSRKMKPKPVQVPGGVVLVDDTFTIKSGGSKRTADGSEFESPKKKRKV

SEQ ID NO: 193 - protein, 781 aa, synthetic construct
MSPSKKNGRSGPQPHKRWVFTLNNPSEDERKKIRDLPISLFDYFIVGEEGNEEGRTPHLQGFANFVKKQTFNKVKWYLGARCHIEKAKGTDQQNKEYCSKEGNLLMECGAPRSQGQRSGGSSGGSSGSETPGTSESATPESSGGSSGGSSVISYDNYVTILDEETLKAWIAKLEKAPVFAFATATDSLDNISANLVGLSFAIEPGVAAYIPVAHDYLDAPDQISRERALELLKPLLEDEKALKVGQNLKYDRGILANYGIELRGIAFDTMLESYILNSVAGRHDMDSLAERWLKHKTITFEEIAGKGKNQLTFNQIALEEAGRYAAEDADVTLQLHLKMWPDLQKHKGPLNVFENIEMPLVPVLSRIERNGVKIDPKVLHNHSEELTLRLAELEKKAHEIAGEEFNLSSTKQLQTILFEKQGIKPLKKTPGGAPSTSEEVLEELALDYPLPKVILEYRGLAKLKSTYTDKLPLMINPKTGRVHTSYHQAVTATGRLSSTDPNLQNIPVRNEEGRRIRQAFIAPEDYVIVSADYSQIELRIMAHLSRDKGLLTAFAEGKDIHRATAAEVFGLPLETVTSEQRRSAKAINFGLIYGMSAFGLARQLNIPRKEAQKYMDLYFERYPGVLEYMERTRAQAKEQGYVETLDGRRLYLPDIKSSNGARRAAAERAAINAPMQGTAADIIKRAMIAVDAWLQAEQPRVRMIMQVHDELVFEVHKDDVDAVAKQIHQLMENCTRLDVPLLVEVGSGENWDQAHSGGSSGGSKRTADGSEFESPKKKRKV

SEQ ID NO: 194 - protein, 750 aa, synthetic construct
MSPSKKNGRSGPQPHKRWVFTLNNPSEDERKKIRDLPISLFDYFIVGEEGNEEGRTPHLQGFANFVKKQTFNKVKWYLGARCHIEKAKGTDQQNKEYCSKEGNLLMECGAPRSQGQRSGGSSGGSSGSETPGTSESATPESSGGSSGGSSKHMPRKRYSCDFETTTKVEDCRVWAYGYMNIEDHSEYKIGNSLDEFMAWALKVQADLYFHNLKFDGAFIINWLERNGFKWSADGLPNTYNTIISRTGQWYMIDICLGYKGKRKIHTVIYDSLKKLPFPVKKIAKDFKLTVLKGDIDYHKERPVGYKITPEEYAYIKNAIQIIAEALLIQFKQGLDRMTAGSDSLKDFKDIITTKKFKKVFPTLSLGLDKKVRYAYRGGFTWLNDRFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSEGAIKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRPKTYIQDIYMKEVDGELVEGSPDDYTDIKLSVKCAGMTDKIKKEVTFENFKVGFSRKMKPKPVQVPGGVVLVDDTFTIKSGGSSGGSKRTADGSEFESPKKKRKV

SEQ ID NO: 195 - protein, 791 aa, synthetic construct
MGAQPARSMAEAGITGTWYNQSGSTFTVTAGADGNLTGQYENRAQGTGCQNSPYTLTGRYNGTKLEWRVEWNNSTENCHSRTEWRGQYQGGAEARINTQWNLTYEGGSGPATEQGQDTFTKVKPSAASGSSAGGGGSGGGGSGGGGSGPKKKRKVAAAGSVISYDNYVTILDEETLKAWIAKLEKAPVFAFATATDSLDNISANLVGLSFAIEPGVAAYIPVAHDYLDAPDQISRERALELLKPLLEDEKALKVGQNLKYDRGILANYGIELRGIAFDTMLESYILNSVAGRHDMDSLAERWLKHKTITFEEIAGKGKNQLTFNQIALEEAGRYAAEDADVTLQLHLKMWPDLQKHKGPLNVFENIEMPLVPVLSRIERNGVKIDPKVLHNHSEELTLRLAELEKKAHEIAGEEFNLSSTKQLQTILFEKQGIKPLKKTPGGAPSTSEEVLEELALDYPLPKVILEYRGLAKLKSTYTDKLPLMINPKTGRVHTSYHQAVTATGRLSSTDPNLQNIPVRNEEGRRIRQAFIAPEDYVIVSADYSQIELRIMAHLSRDKGLLTAFAEGKDIHRATAAEVFGLPLETVTSEQRRSAKAINFGLIYGMSAFGLARQLNIPRKEAQKYMDLYFERYPGVLEYMERTRAQAKEQGYVETLDGRRLYLPDIKSSNGARRAAAERAAINAPMQGTAADIIKRAMIAVDAWLQAEQPRVRMIMQVHDELVFEVHKDDVDAVAKQIHQLMENCTRLDVPLLVEVGSGENWDQAHSGGSSGGSKRTADGSEFESPKKKRKV

SEQ ID NO: 196 - protein, 760 aa, synthetic construct
MGAQPARSMAEAGITGTWYNQSGSTFTVTAGADGNLTGQYENRAQGTGCQNSPYTLTGRYNGTKLEWRVEWNNSTENCHSRTEWRGQYQGGAEARINTQWNLTYEGGSGPATEQGQDTFTKVKPSAASGSSAGGGGSGGGGSGGGGSGPKKKRKVAAAGSKHMPRKRYSCDFETTTKVEDCRVWAYGYMNIEDHSEYKIGNSLDEFMAWALKVQADLYFHNLKFDGAFIINWLERNGFKWSADGLPNTYNTIISRTGQWYMIDICLGYKGKRKIHTVIYDSLKKLPFPVKKIAKDFKLTVLKGDIDYHKERPVGYKITPEEYAYIKNAIQIIAEALLIQFKQGLDRMTAGSDSLKDFKDIITTKKFKKVFPTLSLGLDKKVRYAYRGGFTWLNDRFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSEGAIKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRPKTYIQDIYMKEVDGELVEGSPDDYTDIKLSVKCAGMTDKIKKEVTFENFKVGFSRKMKPKPVQVPGGVVLVDDTFTIKSGGSSGGSKRTADGSEFESPKKKRKV

SEQ ID NO: 197 - RNA, 110 nt, synthetic construct
  source 1..110: mol_type = other RNA
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 198 - protein, 2191 aa, synthetic construct
MASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYSGGSSGGSSGSETPGTSESATPESSGGSSGGSSDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSKRTADGSEFESPKKKRKVSGGSSGGSVISYDNYVTILDEETLKAWIAKLEKAPVFAFATATDSLDNISANLVGLSFAIEPGVAAYIPVAHDYLDAPDQISRERALELLKPLLEDEKALKVGQNLKYDRGILANYGIELRGIAFDTMLESYILNSVAGRHDMDSLAERWLKHKTITFEEIAGKGKNQLTFNQIALEEAGRYAAEDADVTLQLHLKMWPDLQKHKGPLNVFENIEMPLVPVLSRIERNGVKIDPKVLHNHSEELTLRLAELEKKAHEIAGEEFNLSSTKQLQTILFEKQGIKPLKKTPGGAPSTSEEVLEELALDYPLPKVILEYRGLAKLKSTYTDKLPLMINPKTGRVHTSYHQAVTATGRLSSTDPNLQNIPVRNEEGRRIRQAFIAPEDYVIVSADYSQIELRIMAHLSRDKGLLTAFAEGKDIHRATAAEVFGLPLETVTSEQRRSAKAINFGLIYGMSAFGLARQLNIPRKEAQKYMDLYFERYPGVLEYMERTRAQAKEQGYVETLDGRRLYLPDIKSSNGARRAAAERAAINAPMQGTAADIIKRAMIAVDAWLQAEQPRVRMIMQVHDELVFEVHKDDVDAVAKQIHQLMENCTRLDVPLLVEVGSGENWDQAHSGGSKRTADGSEFESPKKKRKV

SEQ ID NO: 199 - protein, 2160 aa, synthetic construct
MASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYSGGSSGGSSGSETPGTSESATPESSGGSSGGSSDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSKRTADGSEFESPKKKRKVSGGSSGGSKHMPRKRYSCDFETTTKVEDCRVWAYGYMNIEDHSEYKIGNSLDEFMAWALKVQADLYFHNLKFDGAFIINWLERNGFKWSADGLPNTYNTIISRTGQWYMIDICLGYKGKRKIHTVIYDSLKKLPFPVKKIAKDFKLTVLKGDIDYHKERPVGYKITPEEYAYIKNAIQIIAEALLIQFKQGLDRMTAGSDSLKDFKDIITTKKFKKVFPTLSLGLDKKVRYAYRGGFTWLNDRFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSEGAIKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRPKTYIQDIYMKEVDGELVEGSPDDYTDIKLSVKCAGMTDKIKKEVTFENFKVGFSRKMKPKPVQVPGGVVLVDDTFTIKSGGSKRTADGSEFESPKKKRKV

SEQ ID NO: 200 - protein, 794 aa, synthetic construct
MASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYSGGSSGGSSGSETPGTSESATPESSGGSSGGSSVISYDNYVTILDEETLKAWIAKLEKAPVFAFATATDSLDNISANLVGLSFAIEPGVAAYIPVAHDYLDAPDQISRERALELLKPLLEDEKALKVGQNLKYDRGILANYGIELRGIAFDTMLESYILNSVAGRHDMDSLAERWLKHKTITFEEIAGKGKNQLTFNQIALEEAGRYAAEDADVTLQLHLKMWPDLQKHKGPLNVFENIEMPLVPVLSRIERNGVKIDPKVLHNHSEELTLRLAELEKKAHEIAGEEFNLSSTKQLQTILFEKQGIKPLKKTPGGAPSTSEEVLEELALDYPLPKVILEYRGLAKLKSTYTDKLPLMINPKTGRVHTSYHQAVTATGRLSSTDPNLQNIPVRNEEGRRIRQAFIAPEDYVIVSADYSQIELRIMAHLSRDKGLLTAFAEGKDIHRATAAEVFGLPLETVTSEQRRSAKAINFGLIYGMSAFGLARQLNIPRKEAQKYMDLYFERYPGVLEYMERTRAQAKEQGYVETLDGRRLYLPDIKSSNGARRAAAERAAINAPMQGTAADIIKRAMIAVDAWLQAEQPRVRMIMQVHDELVFEVHKDDVDAVAKQIHQLMENCTRLDVPLLVEVGSGENWDQAHSGGSSGGSKRTADGSEFESPKKKRKV

SEQ ID NO: 201 - protein, 763 aa, synthetic construct
MASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYSGGSSGGSSGSETPGTSESATPESSGGSSGGSSKHMPRKRYSCDFETTTKVEDCRVWAYGYMNIEDHSEYKIGNSLDEFMAWALKVQADLYFHNLKFDGAFIINWLERNGFKWSADGLPNTYNTIISRTGQWYMIDICLGYKGKRKIHTVIYDSLKKLPFPVKKIAKDFKLTVLKGDIDYHKERPVGYKITPEEYAYIKNAIQIIAEALLIQFKQGLDRMTAGSDSLKDFKDIITTKKFKKVFPTLSLGLDKKVRYAYRGGFTWLNDRFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSEGAIKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRPKTYIQDIYMKEVDGELVEGSPDDYTDIKLSVKCAGMTDKIKKEVTFENFKVGFSRKMKPKPVQVPGGVVLVDDTFTIKSGGSSGGSKRTADGSEFESPKKKRKV

SEQ ID NO: 202 - protein, 2191 aa, synthetic construct
MASNFTQFVLVDNGGTGDVTVAPSNFANGVAEWISSNSRSQAYKVTCSVRQSSAQNRKYTIKVEVPKVATQTVGGVELPVAAWRSYLNMELTIPIFATNSDCELIVKAMQGLLKDGNPIPSAIAANSGIYSGGSSGGSSGSETPGTSESATPESSGGSSGGSSDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSKRTADGSEFESPKKKRKVSGGSSGGSVISYDNYVTILDEETLKAWIAKLEKAPVFAFATATDSLDNISANLVGLSFAIEPGVAAYIPVAHDYLDAPDQISRERALELLKPLLEDEKALKVGQNLKYDRGILANYGIELRGIAFDTMLESYILNSVAGRHDMDSLAERWLKHKTITFEEIAGKGKNQLTFNQIALEEAGRYAAEDADVTLQLHLKMWPDLQKHKGPLNVFENIEMPLVPVLSRIERNGVKIDPKVLHNHSEELTLRLAELEKKAHEIAGEEFNLSSTKQLQTILFEKQGIKPLKKTPGGAPSTSEEVLEELALDYPLPKVILEYRGLAKLKSTYTDKLPLMINPKTGRVHTSYHQAVTATGRLSSTDPNLQNIPVRNEEGRRIRQAFIAPEDYVIVSADYSQIELRIMAHLSRDKGLLTAFAEGKDIHRATAAEVFGLPLETVTSEQRRSAKAINFGLIYGMSAFGLARQLNIPRKEAQKYMDLYFERYPGVLEYMERTRAQAKEQGYVETLDGRRLYLPDIKSSNGARRAAAERAAINAPMQGTAADIIKRAMIAVDAWLQAEQPRVRMIMQVHDELVFEVHKDDVDAVAKQIHQLMENCTRLDVPLLVEVGSGENWDQAHSGGSKRTADGSEFESPKKKRKV

SEQ ID NO: 203 - protein, 2160 aa, synthetic construct
MASNFTQFVLVDNGGTGDVTVAPSNFANGVAEWISSNSRSQAYKVTCSVRQSSAQNRKYTIKVEVPKVATQTVGGVELPVAAWRSYLNMELTIPIFATNSDCELIVKAMQGLLKDGNPIPSAIAANSGIYSGGSSGGSSGSETPGTSESATPESSGGSSGGSSDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSKRTADGSEFESPKKKRKVSGGSSGGSKHMPRKRYSCDFETTTKVEDCRVWAYGYMNIEDHSEYKIGNSLDEFMAWALKVQADLYFHNLKFDGAFIINWLERNGFKWSADGLPNTYNTIISRTGQWYMIDICLGYKGKRKIHTVIYDSLKKLPFPVKKIAKDFKLTVLKGDIDYHKERPVGYKITPEEYAYIKNAIQIIAEALLIQFKQGLDRMTAGSDSLKDFKDIITTKKFKKVFPTLSLGLDKKVRYAYRGGFTWLNDRFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSEGAIKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRPKTYIQDIYMKEVDGELVEGSPDDYTDIKLSVKCAGMTDKIKKEVTFENFKVGFSRKMKPKPVQVPGGVVLVDDTFTIKSGGSKRTADGSEFESPKKKRKV

SEQ ID NO: 204 - protein, 2147 aa, synthetic construct
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSKRTADGSEFESPKKKRKVSGGSSGGSVISYDNYVTILDEETLKAWIAKLEKAPVFAFATATDSLDNISANLVGLSFAIEPGVAAYIPVAHDYLDAPDQISRERALELLKPLLEDEKALKVGQNLKYDRGILANYGIELRGIAFDTMLESYILNSVAGRHDMDSLAERWLKHKTITFEEIAGKGKNQLTFNQIALEEAGRYAAEDADVTLQLHLKMWPDLQKHKGPLNVFENIEMPLVPVLSRIERNGVKIDPKVLHNHSEELTLRLAELEKKAHEIAGEEFNLSSTKQLQTILFEKQGIKPLKKTPGGAPSTSEEVLEELALDYPLPKVILEYRGLAKLKSTYTDKLPLMINPKTGRVHTSYHQAVTATGRLSSTDPNLQNIPVRNEEGRRIRQAFIAPEDYVIVSADYSQIELRIMAHLSRDKGLLTAFAEGKDIHRATAAEVFGLPLETVTSEQRRSAKAINFGLIYGMSAFGLARQLNIPRKEAQKYMDLYFERYPGVLEYMERTRAQAKEQGYVETLDGRRLYLPDIKSSNGARRAAAERAAINAPMQGTAADIIKRAMIAVDAWLQAEQPRVRMIMQVHDELVFEVHKDDVDAVAKQIHQLMENCTRLDVPLLVEVGSGENWDQAHSGGSSGSETPGTSESATPESSGGSSPSKKNGRSGPQPHKRWVFTLNNPSEDERKKIRDLPISLFDYFIVGEEGNEEGRTPHLQGFANFVKKQTFNKVKWYLGARCHIEKAKGTDQQNKEYCSKEGNLLMECGAPRSQGQR

SEQ ID NO: 205 - protein, 2169 aa, synthetic construct
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGSETPGTSESATPESSGGSSPSKKNGRSGPQPHKRWVFTLNNPSEDERKKIRDLPISLFDYFIVGEEGNEEGRTPHLQGFANFVKKQTFNKVKWYLGARCHIEKAKGTDQQNKEYCSKEGNLLMECGAPRSQGQRSGGSSGGSKRTADGSEFESPKKKRKVSGGSSGGSVISYDNYVTILDEETLKAWIAKLEKAPVFAFATATDSLDNISANLVGLSFAIEPGVAAYIPVAHDYLDAPDQISRERALELLKPLLEDEKALKVGQNLKYDRGILANYGIELRGIAFDTMLESYILNSVAGRHDMDSLAERWLKHKTITFEEIAGKGKNQLTFNQIALEEAGRYAAEDADVTLQLHLKMWPDLQKHKGPLNVFENIEMPLVPVLSRIERNGVKIDPKVLHNHSEELTLRLAELEKKAHEIAGEEFNLSSTKQLQTILFEKQGIKPLKKTPGGAPSTSEEVLEELALDYPLPKVILEYRGLAKLKSTYTDKLPLMINPKTGRVHTSYHQAVTATGRLSSTDPNLQNIPVRNEEGRRIRQAFIAPEDYVIVSADYSQIELRIMAHLSRDKGLLTAFAEGKDIHRATAAEVFGLPLETVTSEQRRSAKAINFGLIYGMSAFGLARQLNIPRKEAQKYMDLYFERYPGVLEYMERTRAQAKEQGYVETLDGRRLYLPDIKSSNGARRAAAERAAINAPMQGTAADIIKRAMIAVDAWLQAEQPRVRMIMQVHDELVFEVHKDDVDAVAKQIHQLMENCTRLDVPLLVEVGSGENWDQAHSGGSKRTADGSEFESPKKKRKV

SEQ ID NO: 206 - protein, 2169 aa, synthetic construct
MSPSKKNGRSGPQPHKRWVFTLNNPSEDERKKIRDLPISLFDYFIVGEEGNEEGRTPHLQGFANFVKKQTFNKVKWYLGARCHIEKAKGTDQQNKEYCSKEGNLLMECGAPRSQGQRSGGSSGSETPGTSESATPESSGGSVISYDNYVTILDEETLKAWIAKLEKAPVFAFATATDSLDNISANLVGLSFAIEPGVAAYIPVAHDYLDAPDQISRERALELLKPLLEDEKALKVGQNLKYDRGILANYGIELRGIAFDTMLESYILNSVAGRHDMDSLAERWLKHKTITFEEIAGKGKNQLTFNQIALEEAGRYAAEDADVTLQLHLKMWPDLQKHKGPLNVFENIEMPLVPVLSRIERNGVKIDPKVLHNHSEELTLRLAELEKKAHEIAGEEFNLSSTKQLQTILFEKQGIKPLKKTPGGAPSTSEEVLEELALDYPLPKVILEYRGLAKLKSTYTDKLPLMINPKTGRVHTSYHQAVTATGRLSSTDPNLQNIPVRNEEGRRIRQAFIAPEDYVIVSADYSQIELRIMAHLSRDKGLLTAFAEGKDIHRATAAEVFGLPLETVTSEQRRSAKAINFGLIYGMSAFGLARQLNIPRKEAQKYMDLYFERYPGVLEYMERTRAQAKEQGYVETLDGRRLYLPDIKSSNGARRAAAERAAINAPMQGTAADIIKRAMIAVDAWLQAEQPRVRMIMQVHDELVFEVHKDDVDAVAKQIHQLMENCTRLDVPLLVEVGSGENWDQAHSGGSSGGSKRTADGSEFESPKKKRKVSGGSSGGSDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSKRTADGSEFESPKKKRKV

SEQ ID NO: 207 - protein, 2169 aa, synthetic construct
MVISYDNYVTILDEETLKAWIAKLEKAPVFAFATATDSLDNISANLVGLSFAIEPGVAAYIPVAHDYLDAPDQISRERALELLKPLLEDEKALKVGQNLKYDRGILANYGIELRGIAFDTMLESYILNSVAGRHDMDSLAERWLKHKTITFEEIAGKGKNQLTFNQIALEEAGRYAAEDADVTLQLHLKMWPDLQKHKGPLNVFENIEMPLVPVLSRIERNGVKIDPKVLHNHSEELTLRLAELEKKAHEIAGEEFNLSSTKQLQTILFEKQGIKPLKKTPGGAPSTSEEVLEELALDYPLPKVILEYRGLAKLKSTYTDKLPLMINPKTGRVHTSYHQAVTATGRLSSTDPNLQNIPVRNEEGRRIRQAFIAPEDYVIVSADYSQIELRIMAHLSRDKGLLTAFAEGKDIHRATAAEVFGLPLETVTSEQRRSAKAINFGLIYGMSAFGLARQLNIPRKEAQKYMDLYFERYPGVLEYMERTRAQAKEQGYVETLDGRRLYLPDIKSSNGARRAAAERAAINAPMQGTAADIIKRAMIAVDAWLQAEQPRVRMIMQVHDELVFEVHKDDVDAVAKQIHQLMENCTRLDVPLLVEVGSGENWDQAHSGGSSGGSKRTADGSEFESPKKKRKVSGGSSGGSSPSKKNGRSGPQPHKRWVFTLNNPSEDERKKIRDLPISLFDYFIVGEEGNEEGRTPHLQGFANFVKKQTFNKVKWYLGARCHIEKAKGTDQQNKEYCSKEGNLLMECGAPRSQGQRSGGSSGSETPGTSESATPESSGGSDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSKRTADGSEFESPKKKRKV

SEQ ID NO: 208 - protein, 2147 aa, synthetic construct
MVISYDNYVTILDEETLKAWIAKLEKAPVFAFATATDSLDNISANLVGLSFAIEPGVAAYIPVAHDYLDAPDQISRERALELLKPLLEDEKALKVGQNLKYDRGILANYGIELRGIAFDTMLESYILNSVAGRHDMDSLAERWLKHKTITFEEIAGKGKNQLTFNQIALEEAGRYAAEDADVTLQLHLKMWPDLQKHKGPLNVFENIEMPLVPVLSRIERNGVKIDPKVLHNHSEELTLRLAELEKKAHEIAGEEFNLSSTKQLQTILFEKQGIKPLKKTPGGAPSTSEEVLEELALDYPLPKVILEYRGLAKLKSTYTDKLPLMINPKTGRVHTSYHQAVTATGRLSSTDPNLQNIPVRNEEGRRIRQAFIAPEDYVIVSADYSQIELRIMAHLSRDKGLLTAFAEGKDIHRATAAEVFGLPLETVTSEQRRSAKAINFGLIYGMSAFGLARQLNIPRKEAQKYMDLYFERYPGVLEYMERTRAQAKEQGYVETLDGRRLYLPDIKSSNGARRAAAERAAINAPMQGTAADIIKRAMIAVDAWLQAEQPRVRMIMQVHDELVFEVHKDDVDAVAKQIHQLMENCTRLDVPLLVEVGSGENWDQAHSGGSSGGSKRTADGSEFESPKKKRKVSGGSSGGSDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGSETPGTSESATPESSGGSSPSKKNGRSGPQPHKRWVFTLNNPSEDERKKIRDLPISLFDYFIVGEEGNEEGRTPHLQGFANFVKKQTFNKVKWYLGARCHIEKAKGTDQQNKEYCSKEGNLLMECGAPRSQGQR

SEQ ID NO: 209 - protein, 522 aa, synthetic construct
MKRTADGSEFESPKKKRKVSGGSRALVVIRLSRVTDATTSPERQLESCQQLCAQRGWDVVGVAEDLDVSGAVDPFDRKRRPNLARWLAFEEQPFDVIVAYRVDRLTRSIRHLQQLVHWAEDHKKLVVSATEAHFDTTTPFAAVVIALMGTVAQMELEAIKERNRSAAHFNIRAGKYRGSLPPWGYLPTRVDGEWRLVPDPVQRERILEVYHRVVDNHEPLHLVAHDLNRRGVLSPKDYFAQLQGREPQGREWSATALKRSMISEAMLGYATLNGKTVRDDDGAPLVRAEPILTREQLEALRAELVKTSRAKPAVSTPSLLLRVLFCAVCGEPAYKFAGGGRKHPRYRCRSMGFPKHCGNGTVAMAEWDAFCEEQVLDLLGDAERLEKVWVAGSDSAVELAEVNAELVDLTSLIGSPAYRAGSPQREALDARIAALAARQEELEGLEARPSGWEWRETGQRFGDWWREQDTAAKNTWLRSMNVRLTFDVRGGLTRTIDFGDLQEYEQHLRLGSVVERLHTGMS

SEQ ID NO: 210 - DNA, 13 bp, synthetic construct
  source 1..13: mol_type = other DNA
aagtattaccagc

SEQ ID NO: 211 - DNA, 38 bp, synthetic construct
  source 1..38: mol_type = other DNA
ggcttgtcgacgacggcggtctccgtcgtcaggatcat

SEQ ID NO: 212 - RNA, 20 nt, synthetic construct
  source 1..20: mol_type = other RNA
gattcctggtgccagaaaca

SEQ ID NO: 213 - RNA, 20 nt, synthetic construct
  source 1..20: mol_type = other RNA
gtcatcttagtcattacctg

SEQ ID NO: 214 - RNA, 20 nt, synthetic construct
  source 1..20: mol_type = other RNA
gctattctcgcagctcacca

SEQ ID NO: 215 - RNA, 20 nt, synthetic construct
  source 1..20: mol_type = other RNA
caggcctcagcttctccgtg

SEQ ID NO: 216 - RNA, 20 nt, synthetic construct
  source 1..20: mol_type = other RNA
gatgtctgcaggccagatga

SEQ ID NO: 217 - RNA, 20 nt, synthetic construct
  source 1..20: mol_type = other RNA
ccaggtgtctttcaaagtag

SEQ ID NO: 218 - RNA, 20 nt, synthetic construct
  source 1..20: mol_type = other RNA
gcagtggtggggggccttgg

SEQ ID NO: 219 - RNA, 20 nt, synthetic construct
  source 1..20: mol_type = other RNA
acgctgcagacgatcctggg

SEQ ID NO: 220 - RNA, 21 nt, synthetic construct
  source 1..21: mol_type = other RNA
gggcccagactgagcacgtga

SEQ ID NO: 221 - RNA, 21 nt, synthetic construct
  source 1..21: mol_type = other RNA
gcaaccacaaacccacgaggg

SEQ ID NO: 222 - RNA, 20 nt, synthetic construct
  source 1..20: mol_type = other RNA
cccttcagctaaaataaagg

SEQ ID NO: 223 - RNA, 21 nt, synthetic construct
  source 1..21: mol_type = other RNA
gtgccctcccgtcaccgtttc

SEQ ID NO: 224 - RNA, 20 nt, synthetic construct
  source 1..20: mol_type = other RNA
tgagttacaacgaacaccgc

SEQ ID NO: 225 - RNA, 21 nt, synthetic construct
  source 1..21: mol_type = other RNA
gtcaaccattaagcaaaacat

SEQ ID NO: 226 - RNA, 20 nt, synthetic construct
  source 1..20: mol_type = other RNA
gaagccggccttgcacatgc

SEQ ID NO: 227 - RNA, 20 nt, synthetic construct
  source 1..20: mol_type = other RNA
cgcggcgatatcatcatcga

SEQ ID NO: 228 - RNA, 20 nt, synthetic construct
  source 1..20: mol_type = other RNA
ggcatcgtcgcccgcgaagc

SEQ ID NO: 229 - RNA, 20 nt, synthetic construct
  source 1..20: mol_type = other RNA
ggaggggaagacggcccggg

SEQ ID NO: 230 - RNA, 20 nt, synthetic construct
  source 1..20: mol_type = other RNA
tggaggggaagacggcccgg

SEQ ID NO: 231 - RNA, 20 nt, synthetic construct
  source 1..20: mol_type = other RNA
agctgtacacggaccatgca

SEQ ID NO: 232 - RNA, 20 nt, synthetic construct
  source 1..20: mol_type = other RNA
gcatgttttcacgatagtaa

SEQ ID NO: 233 - RNA, 20 nt, synthetic construct
  source 1..20: mol_type = other RNA
ctccctccaagttgtccacg

SEQ ID NO: 234 - RNA, 20 nt, synthetic construct
  source 1..20: mol_type = other RNA
aagacgaatgcacacaacac

SEQ ID NO: 235 - RNA, 21 nt, synthetic construct
  source 1..21: mol_type = other RNA
ggtcaaccagtatcccggtgc

SEQ ID NO: 236 - RNA, 21 nt, synthetic construct
  source 1..21: mol_type = other RNA
gaagcctggccagggagtggc

SEQ ID NO: 237 - RNA, 21 nt, synthetic construct
  source 1..21: mol_type = other RNA
ggcctccccaaagcctggcca

SEQ ID NO: 238 - RNA, 29 nt, synthetic construct
  source 1..29: mol_type = other RNA
atatcatcatcgatggtgagctgcgagaa

SEQ ID NO: 239 - RNA, 26 nt, synthetic construct
  source 1..26: mol_type = other RNA
ccatcgatgatgatgagctgcgagaa

SEQ ID NO: 240 - RNA, 23 nt, synthetic construct
  source 1..23: mol_type = other RNA
ccatcgatgatgagctgcgagaa

SEQ ID NO: 241 - RNA, 25 nt, synthetic construct
  source 1..25: mol_type = other RNA
tcccgtcaccgtttctggcaccagg

SEQ ID NO: 242 - RNA, 35 nt, synthetic construct
  source 1..35: mol_type = other RNA
aatgtgccatctggcgtacgcatctggcctgcaga

SEQ ID NO: 243 - RNA, 29 nt, synthetic construct
  source 1..29: mol_type = other RNA
aacgaacaccgcaggtaatgactaagatg

SEQ ID NO: 244 - RNA, 20 nt, synthetic construct
  source 1..20: mol_type = other RNA
aatgtaagtattaccagcaa

SEQ ID NO: 245 - RNA, 20 nt, synthetic construct
  source 1..20: mol_type = other RNA
ttgggaagtattaccagcat

SEQ ID NO: 246 - RNA, 20 nt, synthetic construct
  source 1..20: mol_type = other RNA
tttctaagtattaccagccc

SEQ ID NO: 247 - RNA, 20 nt, synthetic construct
  source 1..20: mol_type = other RNA
cacacaagtattaccagctt

SEQ ID NO: 248 - RNA, 20 nt, synthetic construct
  source 1..20: mol_type = other RNA
ctaaccaagtattaccagca

SEQ ID NO: 249 - RNA, 20 nt, synthetic construct
  source 1..20: mol_type = other RNA
aaggctaagtattaccagcc

SEQ ID NO: 250 - RNA, 20 nt, synthetic construct
  source 1..20: mol_type = other RNA
ccttacaagtattaccagcc

SEQ ID NO: 251 - RNA, 20 nt, synthetic construct
  source 1..20: mol_type = other RNA
ggcaagtattaccagcccta

SEQ ID NO: 252 - RNA, 20 nt, synthetic construct
  source 1..20: mol_type = other RNA
aaaaagtattaccagcagaa

SEQ ID NO: 253 - RNA, 20 nt, synthetic construct
  source 1..20: mol_type = other RNA
atgaagtattaccagcatcc

SEQ ID NO: 254 - RNA, 20 nt, synthetic construct
  source 1..20: mol_type = other RNA
gggcaagtattaccagccct

SEQ ID NO: 255 - RNA, 20 nt, synthetic construct
  source 1..20: mol_type = other RNA
cgtaaagtattaccagccgt

SEQ ID NO: 256 - RNA, 20 nt, synthetic construct
  source 1..20: mol_type = other RNA
tgggaagtattaccagcata

SEQ ID NO: 257 - RNA, 20 nt, synthetic construct
  source 1..20: mol_type = other RNA
aagtattaccagcagaatgg

SEQ ID NO: 258 - RNA, 20 nt, synthetic construct
  source 1..20: mol_type = other RNA
aagtattaccagcccttcat

SEQ ID NO: 259 - RNA, 20 nt, synthetic construct
  source 1..20: mol_type = other RNA
aagtattaccagcaattatt

SEQ ID NO: 260 - DNA, 54 bp, synthetic construct
  source 1..54: mol_type = other DNA
acactctttccctacacgacgctcttccgatctcacaacagcttcatgtcagcc

SEQ ID NO: 261 - DNA, 54 bp, synthetic construct
  source 1..54: mol_type = other DNA
gactggagttcagacgtgtgctcttccgatctcgttaatgtttcctgatggtcc

SEQ ID NO: 262 - DNA, 53 bp, synthetic construct
  source 1..53: mol_type = other DNA
acactctttccctacacgacgctcttccgatctgaccatagcacttcccttcc

SEQ ID NO: 263 - DNA, 55 bp, synthetic construct
  source 1..55: mol_type = other DNA
gactggagttcagacgtgtgctcttccgatctttagccaacatacagaagtcagg

SEQ ID NO: 264 - DNA, 52 bp, synthetic construct
  source 1..52: mol_type = other DNA
acactctttccctacacgacgctcttccgatctggcgacttcggctcacagc

SEQ ID NO: 265 - DNA, 51 bp, synthetic construct
  source 1..51: mol_type = other DNA
gactggagttcagacgtgtgctcttccgatctacccagccagctcccctac

SEQ ID NO: 266 - DNA, 53 bp, synthetic construct
  source 1..53: mol_type = other DNA
acactctttccctacacgacgctcttccgatctcgaagatggtgaagctgccg

SEQ ID NO: 267 - DNA, 53 bp, synthetic construct
  source 1..53: mol_type = other DNA
gactggagttcagacgtgtgctcttccgatctcatccctccttctgtcttctg

SEQ ID NO: 268 - DNA, 55 bp, synthetic construct
  source 1..55: mol_type = other DNA
acactctttccctacacgacgctcttccgatctacttggtgccaaattcttctcc

SEQ ID NO: 269 - DNA, 51 bp, synthetic construct
  source 1..51: mol_type = other DNA
gactggagttcagacgtgtgctcttccgatctagagggaatgggctttgga

SEQ ID NO: 270 - DNA, 53 bp, synthetic construct
  source 1..53: mol_type = other DNA
acactctttccctacacgacgctcttccgatctgtcagtggaacaagccgagt

SEQ ID NO: 271 - DNA, 54 bp, synthetic construct
  source 1..54: mol_type = other DNA
gactggagttcagacgtgtgctcttccgatctgtgaccgtgtgctgcttgattg

SEQ ID NO: 272 - DNA, 55 bp, synthetic construct
  source 1..55: mol_type = other DNA
acactctttccctacacgacgctcttccgatctggaccaggacaggaaggaggaa

SEQ ID NO: 273 - DNA, 52 bp, synthetic construct
  source 1..52: mol_type = other DNA
gactggagttcagacgtgtgctcttccgatctggcaggtacaaagtgggagg

SEQ ID NO: 274 - DNA, 52 bp, synthetic construct
  source 1..52: mol_type = other DNA
acactctttccctacacgacgctcttccgatctctgctgcaagtaagcatgc

SEQ ID NO: 275 - DNA, 52 bp, synthetic construct
  source 1..52: mol_type = other DNA
gactggagttcagacgtgtgctcttccgatctcctccctaggtgctggcttc

SEQ ID NO: 276 - DNA, 53 bp, synthetic construct
  source 1..53: mol_type = other DNA
acactctttccctacacgacgctcttccgatctgaagcaggccaatggggagg

SEQ ID NO: 277 - DNA, 54 bp, synthetic construct
  source 1..54: mol_type = other DNA
gactggagttcagacgtgtgctcttccgatctcttgtccctctgtcaatggcgg

SEQ ID NO: 278 - DNA, 53 bp, synthetic construct
  source 1..53: mol_type = other DNA
acactctttccctacacgacgctcttccgatcttctgtgagaggggtggagcc

SEQ ID NO: 279 - DNA, 53 bp, synthetic construct
  source 1..53: mol_type = other DNA
gactggagttcagacgtgtgctcttccgatctccaggctactgcagtagggga

SEQ ID NO: 280 - DNA, 53 bp, synthetic construct
  source 1..53: mol_type = other DNA
acactctttccctacacgacgctcttccgatctgtgtctctggcctgtcctgc

SEQ ID NO: 281 - DNA, 52 bp, synthetic construct
  source 1..52: mol_type = other DNA
gactggagttcagacgtgtgctcttccgatctcccatgaggtgtggcaagga

SEQ ID NO: 282 - DNA, 53 bp, synthetic construct
  source 1..53: mol_type = other DNA
acactctttccctacacgacgctcttccgatcttgccagtcacccaagtctgg

SEQ ID NO: 283 - DNA, 52 bp, synthetic construct
  source 1..52: mol_type = other DNA
gactggagttcagacgtgtgctcttccgatcttcttgactgtggcctccaga

SEQ ID NO: 284 - DNA, 53 bp, synthetic construct
  source 1..53: mol_type = other DNA
acactctttccctacacgacgctcttccgatctgctgctcaggcctttgacca

SEQ ID NO: 285 - DNA, 52 bp, synthetic construct
  source 1..52: mol_type = other DNA
gactggagttcagacgtgtgctcttccgatctccaacctgcggcctcacaat

SEQ ID NO: 286 - DNA, 53 bp, synthetic construct
  source 1..53: mol_type = other DNA
acactctttccctacacgacgctcttccgatctattgcaacaggggcaggtcc

SEQ ID NO: 287 - DNA, 52 bp, synthetic construct
  source 1..52: mol_type = other DNA
gactggagttcagacgtgtgctcttccgatcttgcagaacaggcttcccacg

SEQ ID NO: 288 - DNA, 53 bp, synthetic construct
  source 1..53: mol_type = other DNA
acactctttccctacacgacgctcttccgatctgggtctcttctcgcttgggc

SEQ ID NO: 289 - DNA, 52 bp, synthetic construct
  source 1..52: mol_type = other DNA
gactggagttcagacgtgtgctcttccgatctgcgggcagatcacttgagcc

SEQ ID NO: 290 - DNA, 53 bp, synthetic construct
  source 1..53: mol_type = other DNA
acactctttccctacacgacgctcttccgatctagctgctctttgccaaccct

SEQ ID NO: 291 - DNA, 52 bp, synthetic construct
  source 1..52: mol_type = other DNA
gactggagttcagacgtgtgctcttccgatctcggagtcaggcctaacgctg

SEQ ID NO: 292 - DNA, 53 bp, synthetic construct
  source 1..53: mol_type = other DNA
acactctttccctacacgacgctcttccgatctaggaatgtgccctcggttgc

SEQ ID NO: 293 - DNA, 52 bp, synthetic construct
  source 1..52: mol_type = other DNA
gactggagttcagacgtgtgctcttccgatctagttgggtgcttctccaggc

SEQ ID NO: 294 - DNA, 53 bp, synthetic construct
  source 1..53: mol_type = other DNA
acactctttccctacacgacgctcttccgatctactagcccctgatgcactgc

SEQ ID NO: 295 - DNA, 54 bp, synthetic construct
  source 1..54: mol_type = other DNA
gactggagttcagacgtgtgctcttccgatctgcccagattcaaggtagggagg

SEQ ID NO: 296 - DNA, 53 bp, synthetic construct
  source 1..53: mol_type = other DNA
acactctttccctacacgacgctcttccgatctggtggcatgtgagctgaggc

SEQ ID NO: 297 - DNA, 53 bp, synthetic construct
  source 1..53: mol_type = other DNA
gactggagttcagacgtgtgctcttccgatcttgccctgcactacacatgtca

SEQ ID NO: 298 - DNA, 53 bp, synthetic construct
  source 1..53: mol_type = other DNA
acactctttccctacacgacgctcttccgatctagctctgccttccacccaga

SEQ ID NO: 299 - DNA, 52 bp, synthetic construct
  source 1..52: mol_type = other DNA
gactggagttcagacgtgtgctcttccgatctctcgaagtgcccagctggag

SEQ ID NO: 300 - DNA, 53 bp, synthetic construct
  source 1..53: mol_type = other DNA
acactctttccctacacgacgctcttccgatctgggtgagtggggtgaggtga

SEQ ID NO: 301 - DNA, 52 bp, synthetic construct
  source 1..52: mol_type = other DNA
gactggagttcagacgtgtgctcttccgatcttctgcctccctgagcttcca

SEQ ID NO: 302 - DNA, 53 bp, synthetic construct
  source 1..53: mol_type = other DNA
acactctttccctacacgacgctcttccgatctcaggccctgtcctccctcat

SEQ ID NO: 303 - DNA, 52 bp, synthetic construct
  source 1..52: mol_type = other DNA
gactggagttcagacgtgtgctcttccgatctctcacatgagaaaggggctt

SEQ ID NO: 304 - DNA, 53 bp, synthetic construct
  source 1..53: mol_type = other DNA
acactctttccctacacgacgctcttccgatcttcccgccctaagtcacctca

SEQ ID NO: 305 - DNA, 54 bp, synthetic construct
  source 1..54: mol_type = other DNA
gactggagttcagacgtgtgctcttccgatctagggaagtggggaactgaacct

SEQ ID NO: 306 - DNA, 53 bp, synthetic construct
  source 1..53: mol_type = other DNA
acactctttccctacacgacgctcttccgatctccctttccagccagccagac

SEQ ID NO: 307 - DNA, 52 bp, synthetic construct
  source 1..52: mol_type = other DNA
gactggagttcagacgtgtgctcttccgatctacatgtggaaatggagggcc

SEQ ID NO: 308 - DNA, 53 bp, synthetic construct
  source 1..53: mol_type = other DNA
acactctttccctacacgacgctcttccgatcttctctgtttgccctgcacca

SEQ ID NO: 309 - DNA, 52 bp, synthetic construct
  source 1..52: mol_type = other DNA
gactggagttcagacgtgtgctcttccgatctcctacggaagcaacgtggca

SEQ ID NO: 310 - DNA, 54 bp, synthetic construct
  source 1..54: mol_type = other DNA
acactctttccctacacgacgctcttccgatctaggtgaccagaaaagtcttgt

SEQ ID NO: 311 - DNA, 52 bp, synthetic construct
  source 1..52: mol_type = other DNA
gactggagttcagacgtgtgctcttccgatcttcagtcgcagctcaggtctt

SEQ ID NO: 312 - DNA, 53 bp, synthetic construct
  source 1..53: mol_type = other DNA
acactctttccctacacgacgctcttccgatctcctgctctcagctgccagtg

SEQ ID NO: 313 - DNA, 52 bp, synthetic construct
  source 1..52: mol_type = other DNA
gactggagttcagacgtgtgctcttccgatcttggaaaccaggcacccacat

SEQ ID NO: 314 - DNA, 53 bp, synthetic construct
  source 1..53: mol_type = other DNA
acactctttccctacacgacgctcttccgatctcggcacactatgaggctccc

SEQ ID NO: 315 - DNA, 52 bp, synthetic construct
  source 1..52: mol_type = other DNA
gactggagttcagacgtgtgctcttccgatctggccatcttttagcctgggt

SEQ ID NO: 316 - DNA, 53 bp, synthetic construct
  source 1..53: mol_type = other DNA
acactctttccctacacgacgctcttccgatctccaggtctgggctggaagag

SEQ ID NO: 317 - DNA, 52 bp, synthetic construct
  source 1..52: mol_type = other DNA
gactggagttcagacgtgtgctcttccgatctgatgtcctcatggccccagc

SEQ ID NO: 318 - DNA, 55 bp, synthetic construct
  source 1..55: mol_type = other DNA
acactctttccctacacgacgctcttccgatctagaggaggccaaagagctagct

SEQ ID NO: 319 - DNA, 58 bp, synthetic construct
  source 1..58: mol_type = other DNA
gactggagttcagacgtgtgctcttccgatctagtttggtgtcttagttcaggttggt

SEQ ID NO: 320 - DNA, 55 bp, synthetic construct
  source 1..55: mol_type = other DNA
acactctttccctacacgacgctcttccgatctcagaggatgatgaagctgtgct

SEQ ID NO: 321 - DNA, 55 bp, synthetic construct
  source 1..55: mol_type = other DNA
gactggagttcagacgtgtgctcttccgatctgcacacacagaacaggagaatga

SEQ ID NO: 322 - DNA, 53 bp, synthetic construct
  source 1..53: mol_type = other DNA
acactctttccctacacgacgctcttccgatctgtgaaccaggcagggatggg

SEQ ID NO: 323 - DNA, 52 bp, synthetic construct
  source 1..52: mol_type = other DNA
gactggagttcagacgtgtgctcttccgatctacaagccctggagtcccctt

SEQ ID NO: 324 - DNA, 55 bp, synthetic construct
  source 1..55: mol_type = other DNA
acactctttccctacacgacgctcttccgatctggcccaagatggaaggaatggt

SEQ ID NO: 325 - DNA, 52 bp, synthetic construct
  source 1..52: mol_type = other DNA
gactggagttcagacgtgtgctcttccgatctaagctcagcaggccaccaag

SEQ ID NO: 326 - DNA, 53 bp, synthetic construct
  source 1..53: mol_type = other DNA
acactctttccctacacgacgctcttccgatctgccaaggctctgactcggtc

SEQ ID NO: 327 - DNA, 52 bp, synthetic construct
  source 1..52: mol_type = other DNA
gactggagttcagacgtgtgctcttccgatctgcttggcctccagctgtacc

SEQ ID NO: 328 - DNA, 56 bp, synthetic construct
  source 1..56: mol_type = other DNA
acactctttccctacacgacgctcttccgatcttcaactctctggctttgtgacat

SEQ ID NO: 329 - DNA, 52 bp, synthetic construct
  source 1..52: mol_type = other DNA
gactggagttcagacgtgtgctcttccgatctctccacaccactggacccct

SEQ ID NO: 330 - DNA, 53 bp, synthetic construct
  source 1..53: mol_type = other DNA
acactctttccctacacgacgctcttccgatcttggaccttccctcatgccca

SEQ ID NO: 331 - DNA, 52 bp, synthetic construct
  source 1..52: mol_type = other DNA
gactggagttcagacgtgtgctcttccgatctcgcctgccatttccctgtca

SEQ ID NO: 332 - DNA, 54 bp, synthetic construct
  source 1..54: mol_type = other DNA
acactctttccctacacgacgctcttccgatcttgagagtggccaggtctacca

SEQ ID NO: 333 - DNA, 52 bp, synthetic construct
  source 1..52: mol_type = other DNA
gactggagttcagacgtgtgctcttccgatctccactggcccatgtgtcctt

SEQ ID NO: 334 - DNA, 53 bp, synthetic construct
  source 1..53: mol_type = other DNA
acactctttccctacacgacgctcttccgatctcccggcattcctcacttccc

SEQ ID NO: 335 - DNA, 52 bp, synthetic construct
  source 1..52: mol_type = other DNA
gactggagttcagacgtgtgctcttccgatctgtggagcctgcgtgttttgc

SEQ ID NO: 336 - DNA, 58 bp, synthetic construct
  source 1..58: mol_type = other DNA
acactctttccctacacgacgctcttccgatctcatccatagacatacttcgactccc

SEQ ID NO: 337 - DNA, 57 bp, synthetic construct
  source 1..57: mol_type = other DNA
gactggagttcagacgtgtgctcttccgatctccaacttccaatgtacagacaatcc

SEQ ID NO: 338 - DNA, 56 bp, synthetic construct
  source 1..56: mol_type = other DNA
acactctttccctacacgacgctcttccgatctgaccttggcaagctaactcctcc

SEQ ID NO: 339 - DNA, 52 bp, synthetic construct
  source 1..52: mol_type = other DNA
gactggagttcagacgtgtgctcttccgatctgcctgacaagctctccaggc

SEQ ID NO: 340 - DNA, 57 bp, synthetic construct
  source 1..57: mol_type = other DNA
acactctttccctacacgacgctcttccgatctgcgagtttgctcaacaactctgcc

SEQ ID NO: 341 - DNA, 56 bp, synthetic construct
  source 1..56: mol_type = other DNA
gactggagttcagacgtgtgctcttccgatctcacagctagtgagaggaagccaag

SEQ ID NO: 342 - DNA, 57 bp, synthetic construct
  source 1..57: mol_type = other DNA
acactctttccctacacgacgctcttccgatctccatgcggagaacttgcacaacgt

SEQ ID NO: 343 - DNA, 57 bp, synthetic construct
  source 1..57: mol_type = other DNA
gactggagttcagacgtgtgctcttccgatctcacgagaacaccaggtaaacagttc

SEQ ID NO: 344 - DNA, 57 bp, synthetic construct
  source 1..57: mol_type = other DNA
acactctttccctacacgacgctcttccgatctcatcagcccatgaccgacatgtga

SEQ ID NO: 345 - DNA, 56 bp, synthetic construct
  source 1..56: mol_type = other DNA
gactggagttcagacgtgtgctcttccgatctcctgtgggcatttcactgaacatc

SEQ ID NO: 346 - DNA, 56 bp, synthetic construct
  source 1..56: mol_type = other DNA
acactctttccctacacgacgctcttccgatctgaaggtgcaagcttccaggtgtc

SEQ ID NO: 347 - DNA, 57 bp, synthetic construct
  source 1..57: mol_type = other DNA
gactggagttcagacgtgtgctcttccgatctccttacagtgcgatttagggcaaac

SEQ ID NO: 348 - DNA, 59 bp, synthetic construct
  source 1..59: mol_type = other DNA
acactctttccctacacgacgctcttccgatctctatgtctcagtggacaaagagaatc

SEQ ID NO: 349 - DNA, 58 bp, synthetic construct
  source 1..58: mol_type = other DNA
gactggagttcagacgtgtgctcttccgatctgattccaaagtcagaacatgggaagg

SEQ ID NO: 350 - DNA, 57 bp, synthetic construct
  source 1..57: mol_type = other DNA
acactctttccctacacgacgctcttccgatctggcttcctttgcaacctctacatg

SEQ ID NO: 351 - DNA, 56 bp, synthetic construct
  source 1..56: mol_type = other DNA
gactggagttcagacgtgtgctcttccgatctccatagccatattccaagtgccct

SEQ ID NO: 352 - DNA, 64 bp, synthetic construct
  source 1..64: mol_type = other DNA
acactctttccctacacgacgctcttccgatctctcattatggaatcattcatgtgttcttatc

SEQ ID NO: 353 - DNA, 55 bp, synthetic construct
  source 1..55: mol_type = other DNA
gactggagttcagacgtgtgctcttccgatctggagaggcagacagatatgaccc

SEQ ID NO: 354 - DNA, 57 bp, synthetic construct
  source 1..57: mol_type = other DNA
acactctttccctacacgacgctcttccgatctcatccttatcccactgccacacag

SEQ ID NO: 355 - DNA, 57 bp, synthetic construct
  source 1..57: mol_type = other DNA
gactggagttcagacgtgtgctcttccgatctggaaattgctgtggtaccatgggta

SEQ ID NO: 356 - DNA, 57 bp, synthetic construct
  source 1..57: mol_type = other DNA
acactctttccctacacgacgctcttccgatctgattacaggcatgagccaccacac

SEQ ID NO: 357 - DNA, 54 bp, synthetic construct
  source 1..54: mol_type = other DNA
gactggagttcagacgtgtgctcttccgatctcaggtgatctgccgacctttgc

SEQ ID NO: 358 - DNA, 55 bp, synthetic construct
  source 1..55: mol_type = other DNA
acactctttccctacacgacgctcttccgatctccagcctgggtgacagagttag

SEQ ID NO: 359 - DNA, 54 bp, synthetic construct
  source 1..54: mol_type = other DNA
gactggagttcagacgtgtgctcttccgatctcccaccaagatcctgaagcctg

SEQ ID NO: 360 - DNA, 58 bp, synthetic construct
  source 1..58: mol_type = other DNA
acactctttccctacacgacgctcttccgatctctcacaggacatcagattaagctgc

SEQ ID NO: 361 - DNA, 53 bp, synthetic construct
  source 1..53: mol_type = other DNA
gactggagttcagacgtgtgctcttccgatctccaccacacctggccttgaac

SEQ ID NO: 362 - DNA, 56 bp, synthetic construct
  source 1..56: mol_type = other DNA
acactctttccctacacgacgctcttccgatctctttgatctcccttggcttggtc

SEQ ID NO: 363 - DNA, 56 bp, synthetic construct
  source 1..56: mol_type = other DNA
gactggagttcagacgtgtgctcttccgatctggtggtctcctcccttatttgtac

SEQ ID NO: 364 - DNA, 17 bp, synthetic construct
  source 1..17: mol_type = other DNA
ctgttagtattaccacg

SEQ ID NO: 365 - DNA, 17 bp, synthetic construct
  source 1..17: mol_type = other DNA
ctgttaatactatcacg

SEQ ID NO: 366 - DNA, 17 bp, synthetic construct
  source 1..17: mol_type = other DNA
ctgtcagtattaccagc

SEQ ID NO: 367 - DNA, 17 bp, synthetic construct
  source 1..17: mol_type = other DNA
ctgttattattaccagc

SEQ ID NO: 368 - DNA, 17 bp, synthetic construct
  source 1..17: mol_type = other DNA
ctgttagtattaccagc

SEQ ID NO: 369 - DNA, 17 bp, synthetic construct
  source 1..17: mol_type = other DNA
ctgtgagtattaccagc

SEQ ID NO: 370 - DNA, 19 bp, synthetic construct
  source 1..19: mol_type = other DNA
ctgtgtgtgggtgtgcacg

SEQ ID NO: 371 - DNA, 42 bp, synthetic construct
  source 1..42: mol_type = other DNA
  modified_base 40..42: mod_base = OTHER; note = Phosphorothioate linkage
ctgtaagtattaccagctcccgtcaccgtttctggcaccagg

SEQ ID NO: 372 - DNA, 46 bp, synthetic construct
  source 1..46: mol_type = other DNA
  modified_base 44..46: mod_base = OTHER; note = Phosphorothioate linkage
ctgtaagtattaccagcaacgaacaccgcaggtaatgactaagatg

SEQ ID NO: 373 - DNA, 45 bp, synthetic construct
  source 1..45: mol_type = other DNA
  modified_base 43..45: mod_base = OTHER; note = Phosphorothioate linkage
tttgcgtggggtgtggtgcttcccgtcaccgtttctggcaccagg

SEQ ID NO: 374 - DNA, 48 bp, synthetic construct
  source 1..48: mol_type = other DNA
  modified_base 46..48: mod_base = OTHER; note = Phosphorothioate linkage
tgcttccgtactacgaccccccatcccgtcaccgtttctggcaccagg

SEQ ID NO: 375 - DNA, 42 bp, synthetic construct
  source 1..42: mol_type = other DNA
  modified_base 40..42: mod_base = OTHER; note = Phosphorothioate linkage
gttatattattaccggctcccgtcaccgtttctggcaccagg

SEQ ID NO: 376 - DNA, 42 bp, synthetic construct
  source 1..42: mol_type = other DNA
  modified_base 40..42: mod_base = OTHER; note = Phosphorothioate linkage
ggcttagtattaccccctcccgtcaccgtttctggcaccagg

SEQ ID NO: 377 - DNA, 42 bp, synthetic construct
  source 1..42: mol_type = other DNA
  modified_base 40..42: mod_base = OTHER; note = Phosphorothioate linkage
cgtttaatattaccggatcccgtcaccgtttctggcaccagg

SEQ ID NO: 378 - DNA, 42 bp, synthetic construct
  source 1..42: mol_type = other DNA
  modified_base 40..42: mod_base = OTHER; note = Phosphorothioate linkage
ataataatattacgcgctcccgtcaccgtttctggcaccagg

SEQ ID NO: 379 - DNA, 49 bp, synthetic construct
  source 1..49: mol_type = other DNA
  modified_base 47..49: mod_base = OTHER; note = Phosphorothioate linkage
tttgcgtggggtgtggtgctaacgaacaccgcaggtaatgactaagatg

SEQ ID NO: 380 - DNA, 52 bp, synthetic construct
  source 1..52: mol_type = other DNA
  modified_base 50..52: mod_base = OTHER; note = Phosphorothioate linkage
tgcttccgtactacgaccccccaaacgaacaccgcaggtaatgactaagatg

SEQ ID NO: 381 - DNA, 46 bp, synthetic construct
  source 1..46: mol_type = other DNA
  modified_base 44..46: mod_base = OTHER; note = Phosphorothioate linkage
gttatattattaccggcaacgaacaccgcaggtaatgactaagatg

SEQ ID NO: 382 - DNA, 46 bp, synthetic construct
  source 1..46: mol_type = other DNA
  modified_base 44..46: mod_base = OTHER; note = Phosphorothioate linkage
ggcttagtattacccccaacgaacaccgcaggtaatgactaagatg

SEQ ID NO: 383 - DNA, 46 bp, synthetic construct
  source 1..46: mol_type = other DNA
  modified_base 44..46: mod_base = OTHER; note = Phosphorothioate linkage
cgtttaatattaccggaaacgaacaccgcaggtaatgactaagatg

SEQ ID NO: 384 - DNA, 46 bp, synthetic construct
  source 1..46: mol_type = other DNA
  modified_base 44..46: mod_base = OTHER; note = Phosphorothioate linkage
ataataatattacgcgcaacgaacaccgcaggtaatgactaagatg

SEQ ID NO: 385 - DNA, 43 bp, synthetic construct
  source 1..43: mol_type = other DNA
  modified_base 41..43: mod_base = OTHER; note = Phosphorothioate linkage
ctgttagtattaccagcccgtcaccgtttctggcaccaggaat

SEQ ID NO: 386 - DNA, 43 bp, synthetic construct
  source 1..43: mol_type = other DNA
  modified_base 41..43: mod_base = OTHER; note = Phosphorothioate linkage
ctgtcagtattaccagcccgtcaccgtttctggcaccaggaat

SEQ ID NO: 387 - DNA, 43 bp, synthetic construct
  source 1..43: mol_type = other DNA
  modified_base 41..43: mod_base = OTHER; note = Phosphorothioate linkage
ctgttaatactatcagcccgtcaccgtttctggcaccaggaat

SEQ ID NO: 388 - DNA, 43 bp, synthetic construct
  source 1..43: mol_type = other DNA
  modified_base 41..43: mod_base = OTHER; note = Phosphorothioate linkage
ctgttattattaccagcccgtcaccgtttctggcaccaggaat

SEQ ID NO: 389 - DNA, 43 bp, synthetic construct
  source 1..43: mol_type = other DNA
  modified_base 41..43: mod_base = OTHER; note = Phosphorothioate linkage
ctgtgagtattaccagcccgtcaccgtttctggcaccaggaat

SEQ ID NO: 390 - DNA, 45 bp, synthetic construct
  source 1..45: mol_type = other DNA
  modified_base 43..45: mod_base = OTHER; note = Phosphorothioate linkage
ctgtgtgtgggtgtgcagcccgtcaccgtttctggcaccaggaat

SEQ ID NO: 391 - DNA, 52 bp, synthetic construct
  source 1..52: mol_type = other DNA
  modified_base 50..52: mod_base = OTHER; note = Phosphorothioate linkage
ctgtaagtattaccagcgcgatatcatcatcgatggtgagctgcgagaatag

SEQ ID NO: 392 - DNA, 49 bp, synthetic construct
  source 1..49: mol_type = other DNA
  modified_base 47..49: mod_base = OTHER; note = Phosphorothioate linkage
ctgtaagtattaccagccgatatcatcatcgatggtgagctgcgagaat

SEQ ID NO: 393 - DNA, 50 bp, synthetic construct
  source 1..50: mol_type = other DNA
  modified_base 48..50: mod_base = OTHER; note = Phosphorothioate linkage
ctgtaagtattaccagcgcgatatcatcatcgatggtgagctgcgagaat

SEQ ID NO: 394 - DNA, 51 bp, synthetic construct
  source 1..51: mol_type = other DNA
  modified_base 49..51: mod_base = OTHER; note = Phosphorothioate linkage
ctgtaagtattaccagcggcgatatcatcatcgatggtgagctgcgagaat

SEQ ID NO: 395 - DNA, 51 bp, synthetic construct
  source 1..51: mol_type = other DNA
  modified_base 49..51: mod_base = OTHER; note = Phosphorothioate linkage
ctgtaagtattaccagccgatatcatcatcgatggtgagctgcgagaatag

SEQ ID NO: 396 - DNA, 53 bp, synthetic construct
  source 1..53: mol_type = other DNA
  modified_base 51..53: mod_base = OTHER; note = Phosphorothioate linkage
ctgtaagtattaccagcggcgatatcatcatcgatggtgagctgcgagaatag

SEQ ID NO: 397 - DNA, 53 bp, synthetic construct
  source 1..53: mol_type = other DNA
  modified_base 51..53: mod_base = OTHER; note = Phosphorothioate linkage
ctgtaagtattaccagccgatatcatcatcgatggtgagctgcgagaatagcc

SEQ ID NO: 398 - DNA, 54 bp, synthetic construct
  source 1..54: mol_type = other DNA
  modified_base 52..54: mod_base = OTHER; note = Phosphorothioate linkage
ctgtaagtattaccagcgcgatatcatcatcgatggtgagctgcgagaatagcc

SEQ ID NO: 399 - DNA, 55 bp, synthetic construct
  source 1..55: mol_type = other DNA
  modified_base 53..55: mod_base = OTHER; note = Phosphorothioate linkage
ctgtaagtattaccagcggcgatatcatcatcgatggtgagctgcgagaatagcc

SEQ ID NO: 400 - DNA, 48 bp, synthetic construct
  source 1..48: mol_type = other DNA
  modified_base 46..48: mod_base = OTHER; note = Phosphorothioate linkage
ctgtaagtattaccagcatcatcgatggtgagctgcgagaatagccgg

SEQ ID NO: 401 - DNA, 51 bp, synthetic construct
  source 1..51: mol_type = other DNA
  modified_base 49..51: mod_base = OTHER; note = Phosphorothioate linkage
ctgtaagtattaccagcatcatcatcgatggtgagctgcgagaatagccgg

SEQ ID NO: 402 - DNA, 54 bp, synthetic construct
  source 1..54: mol_type = other DNA
  modified_base 52..54: mod_base = OTHER; note = Phosphorothioate linkage
ctgtaagtattaccagcgatatcatcatcgatggtgagctgcgagaatagccgg

SEQ ID NO: 403 - DNA, 55 bp, synthetic construct
  source 1..55: mol_type = other DNA
  modified_base 53..55: mod_base = OTHER; note = Phosphorothioate linkage
ctgtaagtattaccagccgatatcatcatcgatggtgagctgcgagaatagccgg

SEQ ID NO: 404 - DNA, 56 bp, synthetic construct
  source 1..56: mol_type = other DNA
  modified_base 54..56: mod_base = OTHER; note = Phosphorothioate linkage
ctgtaagtattaccagcgcgatatcatcatcgatggtgagctgcgagaatagccgg

SEQ ID NO: 405 - DNA, 57 bp, synthetic construct
  source 1..57: mol_type = other DNA
  modified_base 55..57: mod_base = OTHER; note = Phosphorothioate linkage
ctgtaagtattaccagcggcgatatcatcatcgatggtgagctgcgagaatagccgg

SEQ ID NO: 406 - DNA, 52 bp, synthetic construct
  source 1..52: mol_type = other DNA
  modified_base 50..52: mod_base = OTHER; note = Phosphorothioate linkage
ctgtaagtattaccagcgcgatatcatcatggatgttgagctgcgagaatag

SEQ ID NO: 407 - DNA, 37 bp, synthetic construct
  source 1..37: mol_type = other DNA
  modified_base 35..37: mod_base = OTHER; note = Phosphorothioate linkage
ctgtaagtattaccagcccgtcaccgtttctggcacc

SEQ ID NO: 408 - DNA, 39 bp, synthetic construct
  source 1..39: mol_type = other DNA
  modified_base 37..39: mod_base = OTHER; note = Phosphorothioate linkage
ctgtaagtattaccagcccgtcaccgtttctggcaccag

SEQ ID NO: 409 - DNA, 41 bp, synthetic construct
  source 1..41: mol_type = other DNA
  modified_base 39..41: mod_base = OTHER; note = Phosphorothioate linkage
ctgtaagtattaccagcccgtcaccgtttctggcaccagga

SEQ ID NO: 410 - DNA, 43 bp, synthetic construct
  source 1..43: mol_type = other DNA
  modified_base 41..43: mod_base = OTHER; note = Phosphorothioate linkage
ctgtaagtattaccagcccgtcaccgtttctggcaccaggaat

SEQ ID NO: 411 - DNA, 45 bp, synthetic construct
  source 1..45: mol_type = other DNA
  modified_base 43..45: mod_base = OTHER; note = Phosphorothioate linkage
ctgtaagtattaccagcccgtcaccgtttctggcaccaggaatcc

SEQ ID NO: 412 - DNA, 47 bp, synthetic construct
  source 1..47: mol_type = other DNA
  modified_base 45..47: mod_base = OTHER; note = Phosphorothioate linkage
ctgtaagtattaccagcccgtcaccgtttctggcaccaggaatcccc

SEQ ID NO: 413 - DNA, 40 bp, synthetic construct
  source 1..40: mol_type = other DNA
  modified_base 38..40: mod_base = OTHER; note = Phosphorothioate linkage
ctgtaagtattaccagccccgtcaccgtttctggcaccag

SEQ ID NO: 414 - DNA, 46 bp, synthetic construct
  source 1..46: mol_type = other DNA
  modified_base 44..46: mod_base = OTHER; note = Phosphorothioate linkage
ctgtaagtattaccagccccgtcaccgtttctggcaccaggaatcc

SEQ ID NO: 415 - DNA, 48 bp, synthetic construct
  source 1..48: mol_type = other DNA
  modified_base 46..48: mod_base = OTHER; note = Phosphorothioate linkage
ctgtaagtattaccagcctcccgtcaccgtttctggcaccaggaatcc

SEQ ID NO: 416 - DNA, 51 bp, synthetic construct
  source 1..51: mol_type = other DNA
  modified_base 49..51: mod_base = OTHER; note = Phosphorothioate linkage
ctgtaagtattaccagcgccctcccgtcaccgtttctggcaccaggaatcc

SEQ ID NO: 417 - DNA, 53 bp, synthetic construct
  source 1..53: mol_type = other DNA
  modified_base 51..53: mod_base = OTHER; note = Phosphorothioate linkage
ctgtaagtattaccagcctgccctcccgtcaccgtttctggcaccaggaatcc

SEQ ID NO: 418 - DNA, 49 bp, synthetic construct
  source 1..49: mol_type = other DNA
  modified_base 47..49: mod_base = OTHER; note = Phosphorothioate linkage
ctgtaagtattaccagctcccgtcaccgtttctggcaccaggaatcccc

SEQ ID NO: 419 - DNA, 53 bp, synthetic construct
  source 1..53: mol_type = other DNA
  modified_base 51..53: mod_base = OTHER; note = Phosphorothioate linkage
ctgtaagtattaccagcgccctcccgtcaccgtttctggcaccaggaatcccc

SEQ ID NO: 420 - DNA, 55 bp, synthetic construct
  source 1..55: mol_type = other DNA
  modified_base 53..55: mod_base = OTHER; note = Phosphorothioate linkage
ctgtaagtattaccagcctgccctcccgtcaccgtttctggcaccaggaatcccc

SEQ ID NO: 421 - DNA, 48 bp, synthetic construct
  source 1..48: mol_type = other DNA
  modified_base 46..48: mod_base = OTHER; note = Phosphorothioate linkage
ctgtaagtattaccagcctgtacacggaccatgcacggagaagctgag

SEQ ID NO: 422 - DNA, 50 bp, synthetic construct
  source 1..50: mol_type = other DNA
  modified_base 48..50: mod_base = OTHER; note = Phosphorothioate linkage
ctgtaagtattaccagcctgtacacggaccatgcacggagaagctgaggc

SEQ ID NO: 423 - DNA, 51 bp, synthetic construct
  source 1..51: mol_type = other DNA
  modified_base 49..51: mod_base = OTHER; note = Phosphorothioate linkage
ctgtaagtattaccagctgtacacggaccatgcacggagaagctgaggcct

SEQ ID NO: 424 - DNA, 52 bp, synthetic construct
  source 1..52: mol_type = other DNA
  modified_base 50..52: mod_base = OTHER; note = Phosphorothioate linkage
ctgtaagtattaccagcctgtacacggaccatgcacggagaagctgaggcct

SEQ ID NO: 425 - DNA, 53 bp, synthetic construct
  source 1..53: mol_type = other DNA
  modified_base 51..53: mod_base = OTHER; note = Phosphorothioate linkage
ctgtaagtattaccagcgctgtacacggaccatgcacggagaagctgaggcct

SEQ ID NO: 426 - DNA, 53 bp, synthetic construct
  source 1..53: mol_type = other DNA
  modified_base 51..53: mod_base = OTHER; note = Phosphorothioate linkage
ctgtaagtattaccagctgtacacggaccatgcacggagaagctgaggcctga

SEQ ID NO: 427 - DNA, 54 bp, synthetic construct
  source 1..54: mol_type = other DNA
  modified_base 52..54: mod_base = OTHER; note = Phosphorothioate linkage
ctgtaagtattaccagcctgtacacggaccatgcacggagaagctgaggcctga

SEQ ID NO: 428 - DNA, 55 bp, synthetic construct
  source 1..55: mol_type = other DNA
  modified_base 53..55: mod_base = OTHER; note = Phosphorothioate linkage
ctgtaagtattaccagcgctgtacacggaccatgcacggagaagctgaggcctga

SEQ ID NO: 429 - DNA, 55 bp, synthetic construct
  source 1..55: mol_type = other DNA
  modified_base 53..55: mod_base = OTHER; note = Phosphorothioate linkage
ctgtaagtattaccagctgtacacggaccatgcacggagaagctgaggcctgaga

SEQ ID NO: 430 - DNA, 56 bp, synthetic construct
  source 1..56: mol_type = other DNA
  modified_base 54..56: mod_base = OTHER; note = Phosphorothioate linkage
ctgtaagtattaccagcctgtacacggaccatgcacggagaagctgaggcctgaga

SEQ ID NO: 431 - DNA, 38 bp, synthetic construct
  source 1..38: mol_type = other DNA
  modified_base 36..38: mod_base = OTHER; note = Phosphorothioate linkage
ctgtaagtattaccagcctccgacttctttgaaagaca

SEQ ID NO: 432 - DNA, 40 bp, synthetic construct
  source 1..40: mol_type = other DNA
  modified_base 38..40: mod_base = OTHER; note = Phosphorothioate linkage
ctgtaagtattaccagcctccgacttctttgaaagacacc

SEQ ID NO: 433 - DNA, 42 bp, synthetic construct
  source 1..42: mol_type = other DNA
  modified_base 40..42: mod_base = OTHER; note = Phosphorothioate linkage
ctgtaagtattaccagcctccgacttctttgaaagacacctg

SEQ ID NO: 434 - DNA, 44 bp, synthetic construct
  source 1..44: mol_type = other DNA
  modified_base 42..44: mod_base = OTHER; note = Phosphorothioate linkage
ctgtaagtattaccagcctccgacttctttgaaagacacctgga

SEQ ID NO: 435 - DNA, 46 bp, synthetic construct
  source 1..46: mol_type = other DNA
  modified_base 44..46: mod_base = OTHER; note = Phosphorothioate linkage
ctgtaagtattaccagcctccgacttctttgaaagacacctggagt

SEQ ID NO: 436 - DNA, 48 bp, synthetic construct
  source 1..48: mol_type = other DNA
  modified_base 46..48: mod_base = OTHER; note = Phosphorothioate linkage
ctgtaagtattaccagcctccgacttctttgaaagacacctggagttc

SEQ ID NO: 437 - DNA, 46 bp, synthetic construct
  source 1..46: mol_type = other DNA
  modified_base 44..46: mod_base = OTHER; note = Phosphorothioate linkage
ctgtaagtattaccagcgcctccgacttctttgaaagacacctgga

SEQ ID NO: 438 - DNA, 43 bp, synthetic construct
  source 1..43: mol_type = other DNA
  modified_base 41..43: mod_base = OTHER; note = Phosphorothioate linkage
ctgtaagtattaccagcagcctccgacttctttgaaagacacc

SEQ ID NO: 439 - DNA, 44 bp, synthetic construct
  source 1..44: mol_type = other DNA
  modified_base 42..44: mod_base = OTHER; note = Phosphorothioate linkage
ctgtaagtattaccagcaagcctccgacttctttgaaagacacc

SEQ ID NO: 440 - DNA, 45 bp, synthetic construct
  source 1..45: mol_type = other DNA
  modified_base 43..45: mod_base = OTHER; note = Phosphorothioate linkage
ctgtaagtattaccagccaagcctccgacttctttgaaagacacc

SEQ ID NO: 441 - DNA, 46 bp, synthetic construct
  source 1..46: mol_type = other DNA
  modified_base 44..46: mod_base = OTHER; note = Phosphorothioate linkage
ctgtaagtattaccagcccaagcctccgacttctttgaaagacacc

SEQ ID NO: 442 - DNA, 50 bp, synthetic construct
  source 1..50: mol_type = other DNA
  modified_base 48..50: mod_base = OTHER; note = Phosphorothioate linkage
ctgtaagtattaccagcccaagcctccgacttctttgaaagacacctgga

SEQ ID NO: 443 - DNA, 52 bp, synthetic construct
  source 1..52: mol_type = other DNA
  modified_base 50..52: mod_base = OTHER; note = Phosphorothioate linkage
ctgtaagtattaccagcccaagcctccgacttctttgaaagacacctggagt

SEQ ID NO: 444 - DNA, 51 bp, synthetic construct
  source 1..51: mol_type = other DNA
  modified_base 49..51: mod_base = OTHER; note = Phosphorothioate linkage
ctgtaagtattaccagcctcccaagcctccgacttctttgaaagacacctg

SEQ ID NO: 445 - DNA, 52 bp, synthetic construct
  source 1..52: mol_type = other DNA
  modified_base 50..52: mod_base = OTHER; note = Phosphorothioate linkage
ctgtaagtattaccagctctcccaagcctccgacttctttgaaagacacctg

SEQ ID NO: 446 - DNA, 41 bp, synthetic construct
  source 1..41: mol_type = other DNA
  modified_base 39..41: mod_base = OTHER; note = Phosphorothioate linkage
ctgtaagtattaccagccctccgacttctttgaaagacacc

SEQ ID NO: 447 - DNA, 42 bp, synthetic construct
  source 1..42: mol_type = other DNA
  modified_base 40..42: mod_base = OTHER; note = Phosphorothioate linkage
ctgtaagtattaccagctgtagacgccaaggccccccaccac

SEQ ID NO: 448 - DNA, 41 bp, synthetic construct
  source 1..41: mol_type = other DNA
  modified_base 39..41: mod_base = OTHER; note = Phosphorothioate linkage
ctgtaagtattaccagcatgtagacgccaaggccccccacc

SEQ ID NO: 449 - DNA, 45 bp, synthetic construct
  source 1..45: mol_type = other DNA
  modified_base 43..45: mod_base = OTHER; note = Phosphorothioate linkage
ctgtaagtattaccagcatgtagacgccaaggccccccaccactg

SEQ ID NO: 450 - DNA, 43 bp, synthetic construct
  source 1..43: mol_type = other DNA
  modified_base 41..43: mod_base = OTHER; note = Phosphorothioate linkage
ctgtaagtattaccagcgcatgtagacgccaaggccccccacc

SEQ ID NO: 451 - DNA, 44 bp, synthetic construct
  source 1..44: mol_type = other DNA
  modified_base 42..44: mod_base = OTHER; note = Phosphorothioate linkage
ctgtaagtattaccagcagcatgtagacgccaaggccccccacc

SEQ ID NO: 452 - DNA, 45 bp, synthetic construct
  source 1..45: mol_type = other DNA
  modified_base 43..45: mod_base = OTHER; note = Phosphorothioate linkage
ctgtaagtattaccagccagcatgtagacgccaaggccccccacc

SEQ ID NO: 453 - DNA, 46 bp, synthetic construct
  source 1..46: mol_type = other DNA
  modified_base 44..46: mod_base = OTHER; note = Phosphorothioate linkage
ctgtaagtattaccagcccagcatgtagacgccaaggccccccacc

SEQ ID NO: 454 - DNA, 47 bp, synthetic construct
  source 1..47: mol_type = other DNA
  modified_base 45..47: mod_base = OTHER; note = Phosphorothioate linkage
ctgtaagtattaccagccccagcatgtagacgccaaggccccccacc

SEQ ID NO: 455 - DNA, 48 bp, synthetic construct
  source 1..48: mol_type = other DNA
  modified_base 46..48: mod_base = OTHER; note = Phosphorothioate linkage
ctgtaagtattaccagctcccagcatgtagacgccaaggccccccacc

SEQ ID NO: 456 - DNA, 49 bp, synthetic construct
  source 1..49: mol_type = other DNA
  modified_base 47..49: mod_base = OTHER; note = Phosphorothioate linkage
ctgtaagtattaccagcttcccagcatgtagacgccaaggccccccacc

SEQ ID NO: 457 - DNA, 47 bp, synthetic construct
  source 1..47: mol_type = other DNA
  modified_base 45..47: mod_base = OTHER; note = Phosphorothioate linkage
ctgtaagtattaccagccagcatgtagacgccaaggccccccaccac

SEQ ID NO: 458 - DNA, 51 bp, synthetic construct
  source 1..51: mol_type = other DNA
  modified_base 49..51: mod_base = OTHER; note = Phosphorothioate linkage
ctgtaagtattaccagcttcccagcatgtagacgccaaggccccccaccac

SEQ ID NO: 459 - DNA, 48 bp, synthetic construct
  source 1..48: mol_type = other DNA
  modified_base 46..48: mod_base = OTHER; note = Phosphorothioate linkage
ctgtaagtattaccagcagcatgtagacgccaaggccccccaccactg

SEQ ID NO: 460 - DNA, 52 bp, synthetic construct
  source 1..52: mol_type = other DNA
  modified_base 50..52: mod_base = OTHER; note = Phosphorothioate linkage
ctgtaagtattaccagctcccagcatgtagacgccaaggccccccaccactg

SEQ ID NO: 461 - DNA, 53 bp, synthetic construct
  source 1..53: mol_type = other DNA
  modified_base 51..53: mod_base = OTHER; note = Phosphorothioate linkage
ctgtaagtattaccagcttcccagcatgtagacgccaaggccccccaccactg

SEQ ID NO: 462 - DNA, 55 bp, synthetic construct
  source 1..55: mol_type = other DNA
  modified_base 53..55: mod_base = OTHER; note = Phosphorothioate linkage
ctgtaagtattaccagccagcatgtagacgccaaggccccccaccactgccccag

SEQ ID NO: 463 - DNA, 52 bp, synthetic construct
  source 1..52: mol_type = other DNA
  modified_base 50..52: mod_base = OTHER; note = Phosphorothioate linkage
ctgtaagtattaccagcgcgatatcatcatcgacggtgagctgcgagaatag

SEQ ID NO: 464 - DNA, 52 bp, synthetic construct
  source 1..52: mol_type = other DNA
  modified_base 50..52: mod_base = OTHER; note = Phosphorothioate linkage
ctgtaagtattaccagcgcgatatcatcatcgacgttgagctgcgagaatag

SEQ ID NO: 465 - DNA, 52 bp, synthetic construct
  source 1..52: mol_type = other DNA
  modified_base 50..52: mod_base = OTHER; note = Phosphorothioate linkage
ctgtaagtattaccagcgcgatatcatcatagacgttgagctgcgagaatag

SEQ ID NO: 466 - DNA, 52 bp, synthetic construct
  source 1..52: mol_type = other DNA
  modified_base 50..52: mod_base = OTHER; note = Phosphorothioate linkage
ctgtaagtattaccagcgcgatatcatcagagacgttgagctgcgagaatag

SEQ ID NO: 467 - DNA, 52 bp, synthetic construct
  source 1..52: mol_type = other DNA
  modified_base 50..52: mod_base = OTHER; note = Phosphorothioate linkage
ctgtaagtattaccagcgcgatatcatgagagacgttgagctgcgagaatag

SEQ ID NO: 468 - DNA, 52 bp, synthetic construct
  source 1..52: mol_type = other DNA
  modified_base 50..52: mod_base = OTHER; note = Phosphorothioate linkage
ctgtaagtattaccagcgcgatatcatgagagccgttgagctgcgagaatag

SEQ ID NO: 469 - DNA, 45 bp, synthetic construct
  source 1..45: mol_type = other DNA
  modified_base 43..45: mod_base = OTHER; note = Phosphorothioate linkage
ctgtaagtattaccagccaagcctccgatttctttgaaagacacc

SEQ ID NO: 470 - DNA, 45 bp, synthetic construct
  source 1..45: mol_type = other DNA
  modified_base 43..45: mod_base = OTHER; note = Phosphorothioate linkage
ctgtaagtattaccagccaagcctcagatttctttgaaagacacc

SEQ ID NO: 471 - DNA, 45 bp, synthetic construct
  source 1..45: mol_type = other DNA
  modified_base 43..45: mod_base = OTHER; note = Phosphorothioate linkage
ctgtaagtattaccagccaagcctctgatttctttgaaagacacc

SEQ ID NO: 472 - DNA, 45 bp, synthetic construct
  source 1..45: mol_type = other DNA
  modified_base 43..45: mod_base = OTHER; note = Phosphorothioate linkage
ctgtaagtattaccagccaagcctcggatttctttgaaagacacc

SEQ ID NO: 473 - DNA, 56 bp, synthetic construct
  source 1..56: mol_type = other DNA
  modified_base 54..56: mod_base = OTHER; note = Phosphorothioate linkage
ctgtaagtattaccagcagtgtttgttcacacccccaggatcgtctgcagcgtgcc

SEQ ID NO: 474 - DNA, 57 bp, synthetic construct
  source 1..57: mol_type = other DNA
  modified_base 55..57: mod_base = OTHER; note = Phosphorothioate linkage
ctgtaagtattaccagcgtgtttgttcacacccccaggatcgtctgcagcgtgcccc

SEQ ID NO: 475 - DNA, 58 bp, synthetic construct
  source 1..58: mol_type = other DNA
  modified_base 56..58: mod_base = OTHER; note = Phosphorothioate linkage
ctgtaagtattaccagcagtgtttgttcacacccccaggatcgtctgcagcgtgcccc

SEQ ID NO: 476 - DNA, 40 bp, synthetic construct
  source 1..40: mol_type = other DNA
  modified_base 38..40: mod_base = OTHER; note = Phosphorothioate linkage
ctgtaagtattaccagctctgcgatcacgtgctcagtctg

SEQ ID NO: 477 - DNA, 50 bp, synthetic construct
  source 1..50: mol_type = other DNA
  modified_base 48..50: mod_base = OTHER; note = Phosphorothioate linkage
ctgtaagtattaccagcagcaagcagcactgtgccctcgtgggtttgtgg

SEQ ID NO: 478 - DNA, 48 bp, synthetic construct
  source 1..48: mol_type = other DNA
  modified_base 46..48: mod_base = OTHER; note = Phosphorothioate linkage
ctgtaagtattaccagcgcagcactgtgccctcgtgggtttgtggttg

SEQ ID NO: 479 - DNA, 82 bp, synthetic construct
  source 1..82: mol_type = other DNA
  modified_base 80..82: mod_base = OTHER; note = Phosphorothioate linkage
ctgtaagtattaccagctggaggaagcagggcttcctttcctctgccatcaatgatggtgatgatggtgcgtgctcagtctg

SEQ ID NO: 480 - DNA, 88 bp, synthetic construct
  source 1..88: mol_type = other DNA
  modified_base 86..88: mod_base = OTHER; note = Phosphorothioate linkage
ctgtaagtattaccagctggaggaagcagggcttcctttcctctgccatcacttatcgtcgtcatccttgtaatccgtgctcagtctg

SEQ ID NO: 481 - DNA, 104 bp, synthetic construct
  source 1..104: mol_type = other DNA
  modified_base 102..104: mod_base = OTHER; note = Phosphorothioate linkage
ctgtaagtattaccagctggaggaagcagggcttcctttcctctgccatcaataacttcgtataatgtatgctatacgaagttataacaatcgtgctcagtctg

SEQ ID NO: 482 - DNA, 90 bp, synthetic construct
  source 1..90: mol_type = other DNA
  modified_base 88..90: mod_base = OTHER; note = Phosphorothioate linkage
ctgtaagtattaccagcgatggagccagagaggaattgttataacttcgtatagcatacattatacgaagttatcccagaacctgagctg

SEQ ID NO: 483 - DNA, 90 bp, synthetic construct
  source 1..90: mol_type = other DNA
  modified_base 88..90: mod_base = OTHER; note = Phosphorothioate linkage
ctgtaagtattaccagcgcagctcaggttctgggataacttcgtataatgtatgctatacgaagttataacaattcctctctggctccat

SEQ ID NO: 484 - DNA, 67 bp, synthetic construct
  source 1..67: mol_type = other DNA
  modified_base 65..67: mod_base = OTHER; note = Phosphorothioate linkage
ctgtaagtattaccagctgtcgacgacggcggtctccgtcgtcaggatcattgagctgcgagaatag

SEQ ID NO: 485 - DNA, 50 bp, synthetic construct
  source 1..50: mol_type = other DNA
  modified_base 48..50: mod_base = OTHER; note = Phosphorothioate linkage
tcctgacgacggagaccgccgtcgtcgacaagccgggccgtcttcccctc

SEQ ID NO: 486 - DNA, 52 bp, synthetic construct; identical to SEQ ID NO: 391
  source 1..52: mol_type = other DNA
  modified_base 48..52: mod_base = OTHER; note = Phosphorothioate linkage

SEQ ID NO: 487 - DNA, 52 bp, synthetic construct
  source 1..52: mol_type = other DNA
  modified_base 50..52: mod_base = OTHER; note = Phosphorothioate linkage
ctgtaagtattaccagcgcgatatcatcatcgatggagacctccgagaatag

SEQ ID NO: 488 - DNA, 52 bp, synthetic construct; identical to SEQ ID NO: 391
  source 1..52: mol_type = other DNA
  modified_base 49..52: mod_base = OTHER; note = Phosphorothioate linkage
  modified_base 50..52: mod_base = OTHER; note = 2-Prime-O-methyl nucleotides
  modified_base 50: mod_base = OTHER; note = Uracil

SEQ ID NO: 489 - DNA, 52 bp, synthetic construct; identical to SEQ ID NO: 391
  source 1..52: mol_type = other DNA
  modified_base order(1..4,50..52): mod_base = OTHER; note = Phosphorothioate linkage

SEQ ID NO: 490 - DNA, 52 bp, synthetic construct; identical to SEQ ID NO: 391
  source 1..52: mol_type = other DNA
  modified_base order(1..4,50..52): mod_base = OTHER; note = Phosphorothioate linkage
  modified_base 1..3: mod_base = OTHER; note = 2-Prime-O-methyl nucleotides
  modified_base 2: mod_base = OTHER; note = Uracil

SEQ ID NO: 491 - DNA, 97 bp, synthetic construct
  source 1..97: mol_type = other DNA
ctgtaagtattaccagcgcgatatcatcatcgatggtgagctgcgagaatagaataaacccgcggttctatctagttacgcgttaaaccaactagaa

SEQ ID NO: 492 - DNA, 43 bp, synthetic construct; identical to SEQ ID NO: 410
  source 1..43: mol_type = other DNA
  modified_base 39..43: mod_base = OTHER; note = Phosphorothioate linkage

SEQ ID NO: 493 - DNA, 43 bp, synthetic construct
  source 1..43: mol_type = other DNA
  modified_base 41..43: mod_base = OTHER; note = Phosphorothioate linkage
ctgtaagtattaccagcccgtcaccgtatcagggaccaggaat

SEQ ID NO: 494 - DNA, 43 bp, synthetic construct; identical to SEQ ID NO: 410
  source 1..43: mol_type = other DNA
  modified_base 40..43: mod_base = OTHER; note = Phosphorothioate linkage
  modified_base 41..43: mod_base = OTHER; note = 2-Prime-O-methyl nucleotides
  modified_base 43: mod_base = OTHER; note = Uracil

SEQ ID NO: 495 - DNA, 36 bp, synthetic construct
  source 1..36: mol_type = other DNA
  modified_base 34..36: mod_base = OTHER; note = Phosphorothioate linkage
ggcgatatcatcatcgatggtgagctgcgagaatag

SEQ ID NO: 496 - DNA, 27 bp, synthetic construct
  source 1..27: mol_type = other DNA
  modified_base 25..27: mod_base = OTHER; note = Phosphorothioate linkage
gccgtcaccgtttctggcaccaggaat

SEQ ID NO: 497 - DNA, 52 bp, synthetic construct; identical to SEQ ID NO: 406
  source 1..52: mol_type = other DNA
  modified_base 49..52: mod_base = OTHER; note = Phosphorothioate linkage
  modified_base 50..52: mod_base = OTHER; note = 2-Prime-O-methyl nucleotides
  modified_base 50: mod_base = OTHER; note = Uracil

SEQ ID NO: 498 - DNA, 66 bp, synthetic construct
  source 1..66: mol_type = other DNA
ctgtaagtattaccagcgcgatatcatcatcgatggtgagctgcgagaataggaaactattctccg

SEQ ID NO: 499 - DNA, 68 bp, synthetic construct
  source 1..68: mol_type = other DNA
ctgtaagtattaccagcgcgatatcatcatcgatggtgagctgcgagaataggaaactattctcgctc

SEQ ID NO: 500 - DNA, 70 bp, synthetic construct
  source 1..70: mol_type = other DNA
ctgtaagtattaccagcgcgatatcatcatcgatggtgagctgcgagaataggaaactattctcgcagga

SEQ ID NO: 501 - DNA, 72 bp, synthetic construct
  source 1..72: mol_type = other DNA
ctgtaagtattaccagcgcgatatcatcatcgatggtgagctgcgagaataggaaactattctcgcagctgt

SEQ ID NO: 502 - DNA, 74 bp, synthetic construct
  source 1..74: mol_type = other DNA
ctgtaagtattaccagcgcgatatcatcatcgatggtgagctgcgagaataggaaactattctcgcagctcagg

SEQ ID NO: 503 - DNA, 76 bp, synthetic construct
  source 1..76: mol_type = other DNA
ctgtaagtattaccagcgcgatatcatcatcgatggtgagctgcgagaataggaaactattctcgcagctcaccta

SEQ ID NO: 504 - DNA, 74 bp, synthetic construct
  source 1..74: mol_type = other DNA
ctgtaagtattaccagcgcgatatcatcatcgatggtgagctgcgagaatagccaggtccgaaaggacctggga

SEQ ID NO: 505 - DNA, 78 bp, synthetic construct
  source 1..78: mol_type = other DNA
ctgtaagtattaccagcgcgatatcatcatcgatggtgagctgcgagaatagccaggtccaggaaactggacctggga

SEQ ID NO: 506 - DNA, 80 bp, synthetic construct
  source 1..80: mol_type = other DNA
ctgtaagtattaccagcgcgatatcatcatcgatggtgagctgcgagaatagccaggtccagggaaacctggacctggga

SEQ ID NO: 507 - DNA, 82 bp, synthetic construct
  source 1..82: mol_type = other DNA
ctgtaagtattaccagcgcgatatcatcatcgatggtgagctgcgagaatagccaggtccaggtgaaaacctggacctggga

SEQ ID NO: 508 - DNA, 84 bp, synthetic construct
  source 1..84: mol_type = other DNA
ctgtaagtattaccagcgcgatatcatcatcgatggtgagctgcgagaatagccaggtccaggtcgaaagacctggacctggga

SEQ ID NO: 509 - DNA, 86 bp, synthetic construct
  source 1..86: mol_type = other DNA
ctgtaagtattaccagcgcgatatcatcatcgatggtgagctgcgagaatagccaggtccaggtccgaaaggacctggacctggga

SEQ ID NO: 510 - DNA, 66 bp, synthetic construct
  source 1..66: mol_type = other DNA
ctgtaagtattaccagcgcgatatcatcatcgatggtgagctgcgagaataggaaactattctcgc

SEQ ID NO: 511 - DNA, 72 bp, synthetic construct
  source 1..72: mol_type = other DNA
ctgtaagtattaccagcgcgatatcatcatcgatggtgagctgcgagaataggaaactattctcgcagctca

SEQ ID NO: 512 - DNA, 76 bp, synthetic construct
  source 1..76: mol_type = other DNA
ctgtaagtattaccagcgcgatatcatcatcgatggtgagctgcgagaatagccaggtccaggaaactggacctgg

SEQ ID NO: 513 - DNA, 82 bp, synthetic construct
  source 1..82: mol_type = other DNA
ctgtaagtattaccagcgcgatatcatcatcgatggtgagctgcgagaatagccaggtccaggtcgaaagacctggacctgg

SEQ ID NO: 514 - DNA, 57 bp, synthetic construct
  source 1..57: mol_type = other DNA
ctgtaagtattaccagcccgtcaccgtttctggcaccaggaatgaaaattcctggac

SEQ ID NO: 515 - DNA, 59 bp, synthetic construct
  source 1..59: mol_type = other DNA
ctgtaagtattaccagcccgtcaccgtttctggcaccaggaatgaaaattcctggtggg

SEQ ID NO: 516 - DNA, 61 bp, synthetic construct
  source 1..61: mol_type = other DNA
ctgtaagtattaccagcccgtcaccgtttctggcaccaggaatgaaaattcctggtgcctc

SEQ ID NO: 517 - DNA, 63 bp, synthetic construct
  source 1..63: mol_type = other DNA
ctgtaagtattaccagcccgtcaccgtttctggcaccaggaatgaaaattcctggtgccagtt

SEQ ID NO: 518 - DNA, 65 bp, synthetic construct
  source 1..65: mol_type = other DNA
ctgtaagtattaccagcccgtcaccgtttctggcaccaggaatgaaaattcctggtgccagaatg

SEQ ID NO: 519 - DNA, 67 bp, synthetic construct
  source 1..67: mol_type = other DNA
ctgtaagtattaccagcccgtcaccgtttctggcaccaggaatgaaaattcctggtgccagaaaccc

SEQ ID NO: 520 - DNA, 65 bp, synthetic construct
  source 1..65: mol_type = other DNA
ctgtaagtattaccagcccgtcaccgtttctggcaccaggaatccaggtccgaaaggacctggta

SEQ ID NO: 521 - DNA, 69 bp, synthetic construct
  source 1..69: mol_type = other DNA
ctgtaagtattaccagcccgtcaccgtttctggcaccaggaatccaggtccaggaaactggacctggta

SEQ ID NO: 522 - DNA, 71 bp, synthetic construct
  source 1..71: mol_type = other DNA
ctgtaagtattaccagcccgtcaccgtttctggcaccaggaatccaggtccagggaaacctggacctggta

SEQ ID NO: 523 - DNA, 73 bp, synthetic construct
  source 1..73: mol_type = other DNA
ctgtaagtattaccagcccgtcaccgtttctggcaccaggaatccaggtccaggtgaaaacctggacctggta

SEQ ID NO: 524 - DNA, 75 bp, synthetic construct
  source 1..75: mol_type = other DNA
ctgtaagtattaccagcccgtcaccgtttctggcaccaggaatccaggtccaggtcgaaagacctggacctggta

SEQ ID NO: 525 - DNA, 77 bp, synthetic construct
  source 1..77: mol_type = other DNA
ctgtaagtattaccagcccgtcaccgtttctggcaccaggaatccaggtccaggtccgaaaggacctggacctggta

SEQ ID NO: 526 - DNA, 57 bp, synthetic construct
  source 1..57: mol_type = other DNA
ctgtaagtattaccagcccgtcaccgtttctggcaccaggaatgaaaattcctggtg

SEQ ID NO: 527 - DNA, 63 bp, synthetic construct
  source 1..63: mol_type = other DNA
ctgtaagtattaccagcccgtcaccgtttctggcaccaggaatgaaaattcctggtgccagaa

SEQ ID NO: 528 - DNA, 67 bp, synthetic construct
  source 1..67: mol_type = other DNA
ctgtaagtattaccagcccgtcaccgtttctggcaccaggaatccaggtccaggaaactggacctgg

SEQ ID NO: 529 - DNA, 73 bp, synthetic construct
  source 1..73: mol_type = other DNA
ctgtaagtattaccagcccgtcaccgtttctggcaccaggaatccaggtccaggtcgaaagacctggacctgg

SEQ ID NO: 530 - DNA, 62 bp, synthetic construct
  source 1..62: mol_type = other DNA
ctgtaagtattaccagcaacgaacaccgcaggtaatgactaagatgacgaaagtcatctttc

SEQ ID NO: 531 - DNA, 64 bp, synthetic construct
  source 1..64: mol_type = other DNA
ctgtaagtattaccagcaacgaacaccgcaggtaatgactaagatgacgaaagtcatcttagag

SEQ ID NO: 532 - DNA, 66 bp, synthetic construct
  source 1..66: mol_type = other DNA
ctgtaagtattaccagcaacgaacaccgcaggtaatgactaagatgacgaaagtcatcttagtcta

SEQ ID NO: 533 - DNA, 68 bp, synthetic construct
  source 1..68: mol_type = other DNA
ctgtaagtattaccagcaacgaacaccgcaggtaatgactaagatgacgaaagtcatcttagtcatat

SEQ ID NO: 534 - DNA, 70 bp, synthetic construct
  source 1..70: mol_type = other DNA
ctgtaagtattaccagcaacgaacaccgcaggtaatgactaagatgacgaaagtcatcttagtcattagg

SEQ ID NO: 535 - DNA, 72 bp, synthetic construct
  source 1..72: mol_type = other DNA
ctgtaagtattaccagcaacgaacaccgcaggtaatgactaagatgacgaaagtcatcttagtcattaccac

SEQ ID NO: 536 - DNA, 70 bp, synthetic construct
  source 1..70: mol_type = other DNA
ctgtaagtattaccagcaacgaacaccgcaggtaatgactaagatgacccaggtccgaaaggacctggca

SEQ ID NO: 537 - DNA, 74 bp, synthetic construct
  source 1..74: mol_type = other DNA
ctgtaagtattaccagcaacgaacaccgcaggtaatgactaagatgacccaggtccaggaaactggacctggca

SEQ ID NO: 538 - DNA, 76 bp, synthetic construct
  source 1..76: mol_type = other DNA
ctgtaagtattaccagcaacgaacaccgcaggtaatgactaagatgacccaggtccagggaaacctggacctggca

SEQ ID NO: 539 - DNA, 78 bp, synthetic construct
  source 1..78: mol_type = other DNA
ctgtaagtattaccagcaacgaacaccgcaggtaatgactaagatgacccaggtccaggtgaaaacctggacctggca

SEQ ID NO: 540 - DNA, 80 bp, synthetic construct
  source 1..80: mol_type = other DNA
ctgtaagtattaccagcaacgaacaccgcaggtaatgactaagatgacccaggtccaggtcgaaagacctggacctggca

SEQ ID NO: 541 - DNA, 82 bp, synthetic construct
  source 1..82: mol_type = other DNA
ctgtaagtattaccagcaacgaacaccgcaggtaatgactaagatgacccaggtccaggtccgaaaggacctggacctggca

SEQ ID NO: 542 - DNA, 62 bp, synthetic construct
  source 1..62: mol_type = other DNA
ctgtaagtattaccagcaacgaacaccgcaggtaatgactaagatgacgaaagtcatcttag

SEQ ID NO: 543 - DNA, 68 bp, synthetic construct
  source 1..68: mol_type = other DNA
ctgtaagtattaccagcaacgaacaccgcaggtaatgactaagatgacgaaagtcatcttagtcatta

SEQ ID NO: 544 - DNA, 72 bp, synthetic construct
  source 1..72: mol_type = other DNA
ctgtaagtattaccagcaacgaacaccgcaggtaatgactaagatgacccaggtccaggaaactggacctgg

SEQ ID NO: 545 - DNA, 78 bp, synthetic construct
  source 1..78: mol_type = other DNA
ctgtaagtattaccagcaacgaacaccgcaggtaatgactaagatgacccaggtccaggtcgaaagacctggacctgg

SEQ ID NO: 546 - DNA, 58 bp, synthetic construct
  source 1..58: mol_type = other DNA
  modified_base 56..58: mod_base = OTHER; note = Phosphorothioate linkage
  misc_feature 1..23: note = RNA
  misc_feature 24..58: note = DNA
gcacatgaggatcacccatgtgcgcgatatcatcatcgatggtgagctgcgagaatag

SEQ ID NO: 547 - DNA, 58 bp, synthetic construct; identical to SEQ ID NO: 546
  source 1..58: mol_type = other DNA
  modified_base order(1..4,56..58): mod_base = OTHER; note = Phosphorothioate linkage
  modified_base 1..3: mod_base = OTHER; note = 2-Prime-O-methyl nucleotides
  misc_feature 4..23: note = RNA
  modified_base order(1..3,24..58): mod_base = OTHER; note = DNA

SEQ ID NO: 548 - DNA, 49 bp, synthetic construct
  source 1..49: mol_type = other DNA
  modified_base 47..49: mod_base = OTHER; note = Phosphorothioate linkage
  misc_feature 1..23: note = RNA
  misc_feature 24..49: note = DNA
gcacatgaggatcacccatgtgcccgtcaccgtttctggcaccaggaat

SEQ ID NO: 549 - DNA, 49 bp, synthetic construct; identical to SEQ ID NO: 548
  source 1..49: mol_type = other DNA
  modified_base 1..3: mod_base = OTHER; note = 2-Prime-O-methyl nucleotides
  modified_base order(1..4,47..49): mod_base = OTHER; note = Phosphorothioate linkage
  misc_feature 4..23: note = RNA
  misc_feature order(1..3,24..49): note = DNA

SEQ ID NO: 550 - DNA, 18 bp, synthetic construct
  source 1..18: mol_type = other DNA
aagtattaccagcaaagg

SEQ ID NO: 551 - DNA, 18 bp, synthetic construct
  source 1..18: mol_type = other DNA
aagtattaccagcccagg

SEQ ID NO: 552 - DNA, 18 bp, synthetic construct
  source 1..18: mol_type = other DNA
aagtattaccagcatagg

SEQ ID NO: 553 - DNA, 18 bp, synthetic construct
  source 1..18: mol_type = other DNA
aagtattaccagcctggg

SEQ ID NO: 554 - DNA, 18 bp, synthetic construct
  source 1..18: mol_type = other DNA
aagtattaccagctttgg

SEQ ID NO: 555 - DNA, 17 bp, synthetic construct
  source 1..17: mol_type = other DNA
aagtattaccagcaagg

SEQ ID NO: 556 - DNA, 17 bp, synthetic construct
  source 1..17: mol_type = other DNA
aagtattaccagccagg

SEQ ID NO: 557 - DNA, 17 bp, synthetic construct
  source 1..17: mol_type = other DNA
aagtattaccagcctgg

SEQ ID NO: 558 - DNA, 20 bp, synthetic construct
  source 1..20: mol_type = other DNA
aagtattaccagccctaggg

SEQ ID NO: 559 - DNA, 20 bp, synthetic construct
  source 1..20: mol_type = other DNA
aagtattaccagcaacctgg

SEQ ID NO: 560 - DNA, 20 bp, synthetic construct
  source 1..20: mol_type = other DNA
aagtattaccagcatcctgg

SEQ ID NO: 561 - DNA, 20 bp, synthetic construct
  source 1..20: mol_type = other DNA
aagtattaccagctaattgg

SEQ ID NO: 562 - DNA, 20 bp, synthetic construct
  source 1..20: mol_type = other DNA
aagtattaccagctatttgg

SEQ ID NO: 563 - DNA, 19 bp, synthetic construct
  source 1..19: mol_type = other DNA
aagtattaccagccctagg

SEQ ID NO: 564 - DNA, 19 bp, synthetic construct
  source 1..19: mol_type = other DNA
aagtattaccagccgtagg

SEQ ID NO: 565 - DNA, 19 bp, synthetic construct
  source 1..19: mol_type = other DNA
aagtattaccagcataggg

SEQ ID NO: 566 - DNA, 22 bp, synthetic construct
  source 1..22: mol_type = other DNA
aagtattaccagcaacagtcgg

SEQ ID NO: 567 - DNA, 22 bp, synthetic construct
  source 1..22: mol_type = other DNA
aagtattaccagcaattattgg

SEQ ID NO: 568 - DNA, 23 bp, synthetic construct
  source 1..23: mol_type = other DNA
aagtattaccagcaagaaagagg

SEQ ID NO: 569 - DNA, 23 bp, synthetic construct
  source 1..23: mol_type = other DNA
aagtattaccagcagaatggagg

SEQ ID NO: 570 - DNA, 23 bp, synthetic construct
  source 1..23: mol_type = other DNA
aagtattaccagcccttcatcgg

SEQ ID NO: 571 - DNA, 23 bp, synthetic construct
  source 1..23: mol_type = other DNA
aagtattaccagcaattattggg

SEQ ID NO: 572 - protein, 8 aa, synthetic construct
SGGSSGGS

SEQ ID NO: 573 - protein, 4 aa, synthetic construct
SGGS

SEQ ID NO: 574 - DNA, 39 bp, synthetic construct
  source 1..39: mol_type = other DNA
catggaatcccttctgcagcacctggatcgcttttccga

SEQ ID NO: 575 - DNA, 12 bp, synthetic construct
  source 1..12: mol_type = other DNA
gatgagggctcc

SEQ ID NO: 576 - DNA, 12 bp, synthetic construct
  source 1..12: mol_type = other DNA
gatgagtgctcc

SEQ ID NO: 577 - DNA, 12 bp, synthetic construct
  source 1..12: mol_type = other DNA
gatgagtactcc

SEQ ID NO: 578 - DNA, 12 bp, synthetic construct
  source 1..12: mol_type = other DNA
gatgcgtactcc

SEQ ID NO: 579 - DNA, 12 bp, synthetic construct
  source 1..12: mol_type = other DNA
gatgcgtacgcc

SEQ ID NO: 580 - DNA, 12 bp, synthetic construct
  source 1..12: mol_type = other DNA
gagacgtacgcc

SEQ ID NO: 581 - DNA, 10 bp, synthetic construct
  source 1..10: mol_type = other DNA
agtagtggga

SEQ ID NO: 582 - DNA, 10 bp, synthetic construct
  source 1..10: mol_type = other DNA
agaagtcgga

SEQ ID NO: 583 - DNA, 10 bp, synthetic construct
  source 1..10: mol_type = other DNA
agaaatcgga

SEQ ID NO: 584 - DNA, 10 bp, synthetic construct
  source 1..10: mol_type = other DNA
agaaatctga

SEQ ID NO: 585 - DNA, 10 bp, synthetic construct
  source 1..10: mol_type = other DNA
agaaatcaga

SEQ ID NO: 586 - DNA, 10 bp, synthetic construct
  source 1..10: mol_type = other DNA
agaaatccga

SEQ ID NO: 587 - DNA, 13 bp, synthetic construct
  source 1..13: mol_type = other DNA
caccatggatgat

SEQ ID NO: 588 - DNA, 13 bp, synthetic construct
  source 1..13: mol_type = other DNA
caccatcgatgat

SEQ ID NO: 589 - DNA, 13 bp, synthetic construct
  source 1..13: mol_type = other DNA
caccgtcgatgat

SEQ ID NO: 590 - DNA, 13 bp, synthetic construct
  source 1..13: mol_type = other DNA
caacgtcgatgat

SEQ ID NO: 591 - DNA, 13 bp, synthetic construct
  source 1..13: mol_type = other DNA
caacgtctatgat

SEQ ID NO: 592 - DNA, 13 bp, synthetic construct
  source 1..13: mol_type = other DNA
caacgtctctgat

SEQ ID NO: 593 - DNA, 13 bp, synthetic construct
  source 1..13: mol_type = other DNA
caacgtctctcat

SEQ ID NO: 594 - DNA, 13 bp, synthetic construct
  source 1..13: mol_type = other DNA
caaggtctctcat

SEQ ID NO: 595 - DNA, 10 bp, synthetic construct
  source 1..10: mol_type = other DNA
caccatggat

SEQ ID NO: 596 - DNA, 10 bp, synthetic construct
  source 1..10: mol_type = other DNA
caccatcgat

SEQ ID NO: 597 - DNA, 10 bp, synthetic construct
  source 1..10: mol_type = other DNA
caccatccat

SEQ ID NO: 598 - DNA, 10 bp, synthetic construct
  source 1..10: mol_type = other DNA
caccatctat

SEQ ID NO: 599 - DNA, 10 bp, synthetic construct
  source 1..10: mol_type = other DNA
caccatcaat

SEQ ID NO: 600 - DNA, 10 bp, synthetic construct
  source 1..10: mol_type = other DNA
caccctcgat

SEQ ID NO: 601 - DNA, 10 bp, synthetic construct
  source 1..10: mol_type = other DNA
caccttcgat

SEQ ID NO: 602 - DNA, 10 bp, synthetic construct
  source 1..10: mol_type = other DNA
caccgtcgat

SEQ ID NO: 603 - DNA, 10 bp, synthetic construct
  source 1..10: mol_type = other DNA
cagcatcgat

SEQ ID NO: 604 - DNA, 10 bp, synthetic construct
  source 1..10: mol_type = other DNA
catcatcgat

SEQ ID NO: 605 - DNA, 10 bp, synthetic construct
  source 1..10: mol_type = other DNA
caacatcgat

SEQ ID NO: 606 - DNA, 10 bp, synthetic construct
  source 1..10: mol_type = other DNA
caccgtccat

SEQ ID NO: 607 - DNA, 10 bp, synthetic construct
  source 1..10: mol_type = other DNA
caccctccat

SEQ ID NO: 608 - DNA, 10 bp, synthetic construct
  source 1..10: mol_type = other DNA
caccttccat

SEQ ID NO: 609 - DNA, 10 bp, synthetic construct
  source 1..10: mol_type = other DNA
caccgtctat

SEQ ID NO: 610 - DNA, 10 bp, synthetic construct
  source 1..10: mol_type = other DNA
caccctctat

SEQ ID NO: 611 - DNA, 10 bp, synthetic construct
  source 1..10: mol_type = other DNA
caccttctat

SEQ ID NO: 612 - DNA, 10 bp, synthetic construct
  source 1..10: mol_type = other DNA
caccgtcaat

SEQ ID NO: 613 - DNA, 10 bp, synthetic construct
  source 1..10: mol_type = other DNA
caccctcaat

SEQ ID NO: 614 - DNA, 10 bp, synthetic construct
  source 1..10: mol_type = other DNA
caccttcaat

SEQ ID NO: 615 - DNA, 10 bp, synthetic construct
  source 1..10: mol_type = other DNA
cagcatccat

SEQ ID NO: 616 - DNA, 10 bp, synthetic construct
  source 1..10: mol_type = other DNA
caacatccat

SEQ ID NO: 617 - DNA, 10 bp, synthetic construct
  source 1..10: mol_type = other DNA
catcatccat

SEQ ID NO: 618 - DNA, 10 bp, synthetic construct
  source 1..10: mol_type = other DNA
cagcatctat

SEQ ID NO: 619 - DNA, 10 bp, synthetic construct
  source 1..10: mol_type = other DNA
caacatctat

SEQ ID NO: 620 - DNA, 10 bp, synthetic construct
  source 1..10: mol_type = other DNA
catcatctat

SEQ ID NO: 621 - DNA, 10 bp, synthetic construct
  source 1..10: mol_type = other DNA
cagcatcaat

SEQ ID NO: 622 - DNA, 10 bp, synthetic construct
  source 1..10: mol_type = other DNA
caacatcaat

SEQ ID NO: 623 - DNA, 10 bp, synthetic construct
  source 1..10: mol_type = other DNA
catcatcaat

SEQ ID NO: 624 - DNA, 10 bp, synthetic construct
  source 1..10: mol_type = other DNA
cagcgtcgat

SEQ ID NO: 625 - DNA, 10 bp, synthetic construct
  source 1..10: mol_type = other DNA
catcgtcgat

SEQ ID NO: 626 - DNA, 10 bp, synthetic construct
  source 1..10: mol_type = other DNA
caacgtcgat

SEQ ID NO: 627 - DNA, 10 bp, synthetic construct
  source 1..10: mol_type = other DNA
cagcctcgat

SEQ ID NO: 628 - DNA, 10 bp, synthetic construct
  source 1..10: mol_type = other DNA
catcctcgat

SEQ ID NO: 629 - DNA, 10 bp, synthetic construct
  source 1..10: mol_type = other DNA
caacctcgat

SEQ ID NO: 630 - DNA, 10 bp, synthetic construct
  source 1..10: mol_type = other DNA
cagcttcgat

SEQ ID NO: 631 - DNA, 10 bp, synthetic construct
  source 1..10: mol_type = other DNA
catcttcgat

SEQ ID NO: 632 - DNA, 10 bp, synthetic construct
  source 1..10: mol_type = other DNA
caacttcgat

SEQ ID NO: 633 - DNA, 10 bp, synthetic construct
  source 1..10: mol_type = other DNA
cagcgtccat

SEQ ID NO: 634 - DNA, 10 bp, synthetic construct
  source 1..10: mol_type = other DNA
catcgtccat

SEQ ID NO: 635 - DNA, 10 bp, synthetic construct
  source 1..10: mol_type = other DNA
caacgtccat

SEQ ID NO: 636 - DNA, 10 bp, synthetic construct
  source 1..10: mol_type = other DNA
cagcctccat

SEQ ID NO: 637 - DNA, 10 bp, synthetic construct
  source 1..10: mol_type = other DNA
catcctccat

SEQ ID NO: 638 - DNA, 10 bp, synthetic construct
  source 1..10: mol_type = other DNA
caacctccat

SEQ ID NO: 639 - DNA, 10 bp, synthetic construct
  source 1..10: mol_type = other DNA
cagcttccat

SEQ ID NO: 640 - DNA, 10 bp, synthetic construct
  source 1..10: mol_type = other DNA
catcttccat

SEQ ID NO: 641 - DNA, 10 bp, synthetic construct
  source 1..10: mol_type = other DNA
caacttccat

SEQ ID NO: 642 - DNA, 10 bp, synthetic construct
  source 1..10: mol_type = other DNA
cagcgtctat

SEQ ID NO: 643 - DNA, 10 bp, synthetic construct
  source 1..10: mol_type = other DNA
catcgtctat

SEQ ID NO: 644 - DNA, 10 bp, synthetic construct
  source 1..10: mol_type = other DNA
caacgtctat

SEQ ID NO: 645 - DNA, 10 bp, synthetic construct
  source 1..10: mol_type = other DNA
cagcctctat

SEQ ID NO: 646 - DNA, 10 bp, synthetic construct
  source 1..10: mol_type = other DNA
catcctctat

SEQ ID NO: 647 - DNA, 10 bp, synthetic construct
  source 1..10: mol_type = other DNA
caacctctat

SEQ ID NO: 648 - DNA, 10 bp, synthetic construct
  source 1..10: mol_type = other DNA
cagcttctat

SEQ ID NO: 649 - DNA, 10 bp, synthetic construct
  source 1..10: mol_type = other DNA
catcttctat

SEQ ID NO: 650 - DNA, 10 bp, synthetic construct
  source 1..10: mol_type = other DNA
caacttctat

SEQ ID NO: 651 - DNA, 10 bp, synthetic construct
  source 1..10: mol_type = other DNA
cagcgtcaat

SEQ ID NO: 652 - DNA, 10 bp, synthetic construct
  source 1..10: mol_type = other DNA
catcgtcaat

SEQ ID NO: 653 - DNA, 10 bp, synthetic construct
  source 1..10: mol_type = other DNA
caacgtcaat

SEQ ID NO: 654 - DNA, 10 bp, synthetic construct
  source 1..10: mol_type = other DNA
cagcctcaat

SEQ ID NO: 655 - DNA, 10 bp, synthetic construct
  source 1..10: mol_type = other DNA
catcctcaat

SEQ ID NO: 656 - DNA, 10 bp, synthetic construct
  source 1..10: mol_type = other DNA
caacctcaat

SEQ ID NO: 657 - DNA, 10 bp, synthetic construct
  source 1..10: mol_type = other DNA
cagcttcaat

SEQ ID NO: 658 - DNA, 10 bp, synthetic construct
  source 1..10: mol_type = other DNA
catcttcaat

SEQ ID NO: 659 - DNA, 10 bp, synthetic construct
  source 1..10: mol_type = other DNA
caacttcaat

SEQ ID NO: 660 - DNA, 17 bp, synthetic construct
  source 1..17: mol_type = other DNA
ctgtatgtattaccagc

SEQ ID NO: 661 - DNA, 17 bp, synthetic construct
  source 1..17: mol_type = other DNA
ctgtaggtattaccagc

SEQ ID NO: 662 - DNA, 17 bp, synthetic construct
  source 1..17: mol_type = other DNA
ctgtacgtattaccagc

SEQ ID NO: 663 - DNA, 17 bp, synthetic construct
  source 1..17: mol_type = other DNA
ctgtaaatattaccagc

SEQ ID NO: 664 - DNA, 17 bp, synthetic construct
  source 1..17: mol_type = other DNA
ctgtaattattaccagc

SEQ ID NO: 665 - DNA, 17 bp, synthetic construct
  source 1..17: mol_type = other DNA
ctgtaactattaccagc

SEQ ID NO: 666 - DNA, 17 bp, synthetic construct
  source 1..17: mol_type = other DNA
ctgtaacttttaccagc

SEQ ID NO: 667 - DNA, 17 bp, synthetic construct
  source 1..17: mol_type = other DNA
ctgtaactgttaccagc

SEQ ID NO: 668 - DNA, 17 bp, synthetic construct
  source 1..17: mol_type = other DNA
ctgtaactcttaccagc

SEQ ID NO: 669 - DNA, 17 bp, synthetic construct
  source 1..17: mol_type = other DNA
ctgtaagtaataccagc

SEQ ID NO: 670 - DNA, 17 bp, synthetic construct
  source 1..17: mol_type = other DNA
ctgtaagtactaccagc

SEQ ID NO: 671 - DNA, 17 bp, synthetic construct
  source 1..17: mol_type = other DNA
ctgtaagtagtaccagc

SEQ ID NO: 672 - DNA, 17 bp, synthetic construct
  source 1..17: mol_type = other DNA
ctgtaagtataaccagc

SEQ ID NO: 673 - DNA, 17 bp, synthetic construct
  source 1..17: mol_type = other DNA
ctgtaagtatcaccagc

SEQ ID NO: 674 - DNA, 17 bp, synthetic construct
  source 1..17: mol_type = other DNA
ctgtaagtatgaccagc

SEQ ID NO: 675 - DNA, 17 bp, synthetic construct
  source 1..17: mol_type = other DNA
ctgtaagcattgccagc

SEQ ID NO: 676 - DNA, 17 bp, synthetic construct
  source 1..17: mol_type = other DNA
ctgttagcattgccagc

SEQ ID NO: 677 - DNA, 17 bp, synthetic construct
  source 1..17: mol_type = other DNA
ctgtcagcattgccagc

SEQ ID NO: 678 - DNA, 17 bp, synthetic construct
  source 1..17: mol_type = other DNA
ctgtgagcattgccagc

SEQ ID NO: 679 - DNA, 17 bp, synthetic construct
  source 1..17: mol_type = other DNA
ctgtatgcattgccagc

SEQ ID NO: 680 - DNA, 17 bp, synthetic construct
  source 1..17: mol_type = other DNA
ctgtaggcattgccagc

SEQ ID NO: 681 - DNA, 17 bp, synthetic construct
  source 1..17: mol_type = other DNA
ctgtacgcattgccagc

SEQ ID NO: 682 - DNA, 17 bp, synthetic construct
  source 1..17: mol_type = other DNA
ctgtaaacattgccagc

SEQ ID NO: 683 - DNA, 17 bp, synthetic construct
  source 1..17: mol_type = other DNA
ctgtaatcattgccagc

SEQ ID NO: 684 - DNA, 17 bp, synthetic construct
  source 1..17: mol_type = other DNA
ctgtaaccattgccagc

SEQ ID NO: 685 - DNA, 17 bp, synthetic construct
  source 1..17: mol_type = other DNA
ctgtaagctttgccagc

SEQ ID NO: 686 - DNA, 17 bp, synthetic construct
  source 1..17: mol_type = other DNA
ctgtaagcgttgccagc

SEQ ID NO: 687 - DNA, 17 bp, synthetic construct
  source 1..17: mol_type = other DNA
ctgtaagccttgccagc

SEQ ID NO: 688 - DNA, 17 bp, synthetic construct
  source 1..17: mol_type = other DNA
ctgtaagcaatgccagc

SEQ ID NO: 689 - DNA, 17 bp, synthetic construct
  source 1..17: mol_type = other DNA
ctgtaagcactgccagc

SEQ ID NO: 690 - DNA, 17 bp, synthetic construct
  source 1..17: mol_type = other DNA
ctgtaagcagtgccagc

SEQ ID NO: 691 - DNA, 17 bp, synthetic construct
  source 1..17: mol_type = other DNA
ctgtaagcatagccagc

SEQ ID NO: 692 - DNA, 17 bp, synthetic construct
  source 1..17: mol_type = other DNA
ctgtaagcatcgccagc

SEQ ID NO: 693 - DNA, 17 bp, synthetic construct
  source 1..17: mol_type = other DNA
ctgtaagcatggccagc

SEQ ID NO: 694 - DNA, 11 bp, synthetic construct
  source 1..11: mol_type = other DNA
ctgtaagtatt

SEQ ID NO: 695 - DNA, 43 bp, synthetic construct; identical to SEQ ID NO: 410
  source 1..43: mol_type = other DNA
  modified_base 40..43: mod_base = OTHER; note = Phosphorothioate linkage

SEQ ID NO: 696 - DNA, 52 bp, synthetic construct; identical to SEQ ID NO: 391
  source 1..52: mol_type = other DNA
  modified_base 49..52: mod_base = OTHER; note = Phosphorothioate linkage

SEQ ID NO: 697 - DNA, 72 bp, synthetic construct
  source 1..72: mol_type = other DNA
tgccagaaacaggggtgacgggagggcagaactagtccttagcagcttcctcctcctttattttattttagc

SEQ ID NO: 698 - DNA, 69 bp, synthetic construct
  source 1..69: mol_type = other DNA
tgccagaaacggtgacgggagggcagaactagtccttagcagcttcctcctcctttattttattttagc

SEQ ID NO: 699 - DNA, 56 bp, synthetic construct
  source 1..56: mol_type = other DNA
tgccagaaacggtgacgggagcaccgactcggtgccactttttcaagttgataacg

SEQ ID NO: 700 - DNA, 48 bp, synthetic construct
  source 1..48: mol_type = other DNA
tgccagaaacggtgacgggagcaccgactcggtgccactttttcaagt

SEQ ID NO: 701 - DNA, 57 bp, synthetic construct
  source 1..57: mol_type = other DNA
tgccagaaacggtgacgggagcaccgactccttcctcctcctttattttattttagc

SEQ ID NO: 702 - DNA, 53 bp, synthetic construct
  source 1..53: mol_type = other DNA
tgccagaaacggtgacgggagcaccgcttcctcctcctttattttattttagc

SEQ ID NO: 703 - DNA, 48 bp, synthetic construct
  source 1..48: mol_type = other DNA
cgttatcaacttgaaaaagtggcaccgagtcggtgctcccgtcacctt

SEQ ID NO: 704 - DNA, 27 bp, synthetic construct
  source 1..27: mol_type = other DNA
tgccagaaacggtgacgggctggtaat

SEQ ID NO: 705 - DNA, 23 bp, synthetic construct
  source 1..23: mol_type = other DNA
tgccagaaacggtgacgggctgg

SEQ ID NO: 706 - DNA, 27 bp, synthetic construct
  source 1..27: mol_type = other DNA
ctgtaagtattaccagcccgtcaccgt

SEQ ID NO: 707 - protein, 6 aa, synthetic construct
GGSGGS

SEQ ID NO: 708 - DNA, 10 bp, synthetic construct
  source 1..10: mol_type = other DNA
gaaagaaaga

SEQ ID NO: 709 - DNA, 11 bp, synthetic construct
  source 1..11: mol_type = other DNA
gaaagaaagaa

SEQ ID NO: 710 - DNA, 12 bp, synthetic construct
  source 1..12: mol_type = other DNA
gaaagaaagaaa

SEQ ID NO: 711 - DNA, 13 bp, synthetic construct
  source 1..13: mol_type = other DNA
gaaagaaagaaag

SEQ ID NO: 712 - DNA, 14 bp, synthetic construct
  source 1..14: mol_type = other DNA
gaaagaaagaaaga

SEQ ID NO: 713 - DNA, 16 bp, synthetic construct
  source 1..16: mol_type = other DNA
gaaagaaagaaagaaa

SEQ ID NO: 714 - DNA, 18 bp, synthetic construct
  source 1..18: mol_type = other DNA
gaaagaaagaaagaaaga

(19) United States
(12) Patent Application Publication
(10) Pub. No.: US 2026/0275313 A1
(43) Pub. Date: Sep. 17, 2026

(54) CLICK-TO-INSTALL GENOME EDITING

(71) Applicant: The General Hospital Corporation, Boston, MA (US)

(72) Inventors: Benjamin Kleinstiver, Boston, MA (US); Connor J. Tou, Boston, MA (US); Joana Ferreira Da Silva, Boston, MA (US)

(21) Appl. No.: 19/473,420

(22) PCT Filed: Apr. 8, 2024
(86) PCT No.: PCT/US2024/023562

§ 371 Date: Oct. 7, 2025

Related U.S. Application Data
(60) Provisional application No. 63/495,047, filed on Apr. 7, 2023.
(60) Provisional application No. 63/581,592, filed on Sep. 8, 2023.

(51) Int. Cl. C12N 9/22; C12N 15/11

(52) U.S. Cl. CPC C12N 9/226; C12N 15/111; C07K 2319/80; C12N 2310/20

(57) ABSTRACT

Here we describe click editing, a series of genome editing approaches that combine DNA nickases (including but not limited to RNA-programmable CRISPR nickases) with DNA ligases or DNA polymerases to perform a range of different genomic edits. The edit of interest is encoded on a nucleic acid template or substrate (optionally a ‘click nucleic acid’ (clkNA) or ‘click DNA’ (clkDNA) as described herein), which can be provided in trans or preferentially recruited to the target site via a variety of nucleic acid tethering methods (optionally HUH-family endonucleases (HUHes), Telomere Binding Proteins (TBPs), or DNA- or RNA-binding proteins).

### CLAIM OF PRIORITY

This application is the U.S. National Phase Application under 35 U.S.C. § 371 of International Patent Application No. PCT/US2024/023562, filed on Apr. 8, 2024, which claims the benefit of U.S. Provisional Patent Application Serial Nos. 63/495,047, filed on Apr. 7, 2023, and 63/581,592, filed on Sep. 8, 2023. The entire contents of the foregoing are hereby incorporated by reference.

### FEDERALLY SPONSORED RESEARCH OR DEVELOPMENT

This invention was made with Government support under Grant No. CA281401 awarded by the National Institutes of Health. The Government has certain rights in the invention.

### SEQUENCE LISTING

This application contains a Sequence Listing that has been submitted electronically as an XML file named “29539-0721WO1.XML”. The XML file, created on May 28, 2024, is 905,815 bytes in size. The material in the XML file is hereby incorporated by reference in its entirety.

### TECHNICAL FIELD

Here we describe click editing, a series of genome editing approaches that combine DNA nickases (including but not limited to RNA-programmable CRISPR nickases) with DNA ligases or DNA polymerases to perform a range of different genomic edits. The edit of interest is encoded on a nucleic acid template or substrate (optionally a ‘click nucleic acid’ (clkNA) or ‘click DNA’ (clkDNA) as described herein), which can be provided in trans or preferentially recruited to the target site via a variety of nucleic acid tethering methods (e.g., HUH-family endonucleases (HUHes), Telomere Binding Proteins (TBPs), or DNA- or RNA-binding proteins).

### BACKGROUND

The development of genome editing technologies, including CRISPR-Cas enzymes, has enabled our ability to make customizable modifications to the human genome. CRISPR-Cas enzymes directed by reprogrammable guide RNAs (gRNAs) can initiate genome editing events by catalyzing DNA double-stranded breaks (DSBs) at specified sites in genomes. Subsequent repair of the DSBs by cellular processes can result in gene knockouts or targeted insertion or deletion mutations (indels) via non-homologous end-joining or microhomology-mediated end-joining (NHEJ and MMEJ, respectively)¹. When the DSB is repaired via homology-directed repair (HDR) in the presence of a donor DNA molecule encoding an edit, this can result in knock-in of small or large desired edits¹. Despite these capabilities, the precise modification of small and large DNA sequences in various cells and organisms via nuclease mediated DSBs is challenging and/or can lead to unwanted side effects²⁻⁷. Thus, next-generation technologies that produce targeted DNA modifications directly on the sequence of interest, that are more independent of the cell cycle and expression levels of DNA repair factors, and that minimize DSBs, are critical to overcoming these caveats.

### SUMMARY

Here we describe click editing, a series of genome editing approaches that combine DNA nickases (including RNA-programmable CRISPR enzymes such as nCas9) with other effectors (including DNA ligases or DNA polymerases in Polymerase Click Editors (named interchangeably as PCEs or CEs) and Ligase Click Editors (LCEs)) to perform a range of different genomic edits. The edit of interest is encoded on a nucleic acid template or substrate (optionally a ‘click nucleic acid’ (clkNA), or ‘click DNA’ (clkDNA) as described herein), which can be provided in trans or preferentially recruited to the target site via a variety of nucleic acid tethering methods (e.g., HUH-family endonucleases (HUHes), Telomere Binding Proteins (TBPs), or other enzymes or proteins that bind or interact with single-stranded DNA (ssDNA) or RNA). The DNA binding domain can be any nickase or nuclease, including RNA-programmable DNA nickases or nucleases, including Cas9 and non-Cas enzymes, such as IscB and TnpB, or other classes of DNA nickases or nucleases. Effectors (such as DNA polymerases and ligases) can be further optimized, and additional effectors (such as serine recombinases) can be utilized. Components can be fused or unfused, and if the latter, such components can be recruited via various domains or methods including but not limited to those described herein.

The methods of DNA-templated ligation or polymerization from clkDNAs enable targeted and precise DNA alterations or replacements, including unrestricted types of nucleotide substitutions, small insertions or deletions, as well as exon or gene-sized insertions or deletions, all without intentionally creating DNA DSBs or reliance on HDR. Click editors are a versatile collection of technologies capable of user-specified genomic modification with advantages relative to current technologies and wide applicability across diverse biological applications, including gene editing, molecular approaches, synthetic biology, agriculture, and therapeutics.

Described herein are click editor fusion proteins comprising a DNA binding domain (optionally an RNA-programmable DNA nickase or nuclease), a clkDNA tethering domain, and an effector domain, with optional linkers therebetween, optionally wherein (i) the DNA binding domain, clkDNA tethering domain, and effector domain are fused in any order; or (ii) the clkDNA tethering domain and/or the effector domain is inlaid internally into the DNA binding domain.

In some embodiments, the DNA binding domain (e.g., an RNA-programmable nickase or nuclease) is a non-target strand nickase or a nuclease (e.g., Cas9 nickase or nuclease, Cas12 nickase or nuclease, an IscB nickase or nuclease, a TnpB nickase or nuclease, etc.; see Table C).

In some embodiments, the clkDNA tethering domain is (i) a sequence-specific covalent or non-covalent ssDNA binding domain optionally an HUH endonuclease or telomere binding protein; (ii) a domain that covalently or noncovalently binds a chemical moiety on the clkNA, optionally Avidin, SNAP-tag, CLIP-tag, HALO-tag; or (iii) an RNA-binding domain, optionally an engineered RNA-binding HUH endonuclease or Telomere binding protein, or a Phage coat protein (CP), optionally MCP, PCP, N21p, N22p, BoxB, or Com. In some embodiments, the clkNA tethering domain is an RNA binding protein, e.g., a phage coat protein (CP), a phage antitermination signal, etc. For example, MCP, MCP(N55K), PCP, Com, Phi21 N protein (N^{Phi22})⁸, Phi 22 N protein (N^{Phi22}), lambda N protein (N^{lambda}), an evolved RNA-binding HUH endonuclease⁹, etc.

In some embodiments, the effector domain is a DNA polymerase or ligase.

In some embodiments, the DNA polymerase is a DNA-dependent DNA polymerase of family A, B, C, D, X, or Y, optionally E. coli Klenow (EcKlenow, optionally with the D355A and/or E357A mutations that deactivate its 3′-5′ exonuclease domain); Taq Stoffel; Pol-Beta; Pol-Beta+Sso7d; Phi29 DNA Polymerase (D169A); Sequenase; T4 DNA Polymerase, and E. coli dKlenow (optionally with the D355A, E357A, D705A, and/or D882A mutations), or is a reverse transcriptase, optionally MarathonRT, GsI-IIC RT, and MMLV-RT variants, e.g., PE2 MMLV RT (with D200N, T306K, W313F, T330P, L603W mutations), or MMLV or PE2 MMLV RT truncations (e.g., truncations 2, 5, or 6), MMLV variants encoded in PE6a-PE6g¹⁰ (e.g. or M-MLV RT (T128N, V223Y, D200C, with or without RNaseH domain truncation of M-MLV), Gs RT or Gs RT (A16E, L37P, A123V), Tfl RT or Tfl RT (P70T, G72V, S87G, M102I, K106R, K118R, I128V, L158Q, F269L, A363V, K413E, S492N) or Tfl RT (P70T, G72V, S87G, M102I, K106R, K118R, I128V, L158Q, F269L, A363V, K413E, S492N, K118R, S188K, I260L, S297Q, R288Q), Ec48 RT or Ec48 RT (E60K, K87E, E165D, D243N, R267I, E279K, K318E, K343N), KORV RT, WMSV RT, AVIRE RT, BAEMV RT, PERV RT, MMTV RT, Ty3 RT, GALV RT, SRV2 RT, MPMV RT, Vp96 RT, ASLV RT), RT HFV, HERV, LtrA, HERV-Kcon, Tel4c, GsI-IIC, Ma-Int5, or engineered Marathon (optionally with D14R, N26R, D74R, Ni16K, or N197R mutations).

In some embodiments, the DNA Ligase is T3; T4; T7; ChlV (SplintR); PhiKMV; Vaccinia; or dT4 ligase.

Also provided herein are click DNA (clkDNA) templates, preferably 15-500 nt long, or at least 10, 15, 16, 17, 18, 19, 20, 25, nt long and up to 100, 200, 250, 300, 400, 500, 600, 700, 750, 800, 900, or 1000 nt long, with ranges having endpoints at any of the foregoing vaules, comprising (i) a localization moiety, (ii) a polymerization template (PT) comprising at least one desired edit or an attachment duplex region (ADR) comprising at least one desired edit, and (iii) a flap (or primer) binding region (FBR, also referred to interchangeably herein as primer binding site (PBS)). Preferably the clkNA templates, also called a clkDNA when comprised of DNA bases, comprise RNA, DNA, or both RNA and DNA (FIG. 16). In some embodiments, the clkNA templates described herein are all DNA or partly DNA and partly RNA; the HUH endonuclease sequence is DNA and the rest is RNA; the HUH endonuclease sequence is DNA, all or a portion of the FBR is RNA and the PT is DNA; or the HUH endonuclease sequence is DNA, and all or a portion of the FBR is RNA and all or a portion of the PT is RNA (FIG. 16).

In some embodiments, the clkNA templates comprise one or more chemical modifications, optionally a modified sugar moiety and/or a modified internucleoside linkage.

In some embodiments, the localization moiety is an HUH endonuclease or TBP recognition sequence, biotin, benzylguanine derivative, benzylcytosine derivative, a chloroalkane, or an RNA sequence that binds to an RNA binding protein¹¹⁻¹⁷, e.g., recognition sequence for a phage coat protein (CP) or phage antitermination signal, e.g., MS2, BoxB (boxBP22, boxBPhi21, boxBlambda, etc.), PP7, or Com.

In some embodiments, the polymerization template (PT) comprises a portion that binds to the target genome, e.g., at least 3, 4, 5, 6, 7, 8, 9, or 10 nt long, and up to 100, 200, 250, 300, 400, 500, 600, 700, 750, 800, 900, or 1000 nt long, with ranges having endpoints at any of the foregoing vaules, and a portion that includes at least one desired edit that is at least 1 nt long.

In some embodiments, the ADR comprises a dsDNA portion that comprises a homology to the target genome, e.g., at least 3, 4, 5, 6, 7, 8, 9, or 10 nt long, and up to 500 nt long with at least one desired edit that is at least 1 nt long.

In some embodiments, the flap binding region is complementary to a genomic flap released by a nickase, optionally wherein the flap binding region is 5-50 nt in length, and is immediately 3′ of the PT or ADR.

Also provided herein are compositions comprising: (i) a click editor fusion protein as described herein; (ii) a clkDNA template as described herein, e.g., wherein the localization moiety of the clkDNA template binds to the clkDNA tethering domain of the click editor fusion protein; and (iii) at least one guide RNA that directs the RNA-programmable DNA nickase to a target DNA sequence, and optionally a second guide RNA that directs the RNA-programmable DNA nickase to a target DNA sequence on the opposite strand.

Additionally, provided herein are click editor compositions comprising a DNA binding domain, a clkDNA tethering domain, and an effector domain, optionally wherein the clkDNA tethering domain and the DNA binding domain are in a single fusion protein, and the effector domain is separate, and optionally wherein the effector domain is an endogenous DNA-dependent DNA polymerase or endogenous DNA ligase. In some embodiments, the effector domain is fused to an RNA binding protein, e.g., a phage coat protein (CP), optionally wherein the composition further comprises at least one guide RNA comprising an RNA hairpin sequence that binds the RNA binding protein, e.g., the CP; exemplary pairs include MCP and MS2, PCP and PP7, Phi21 N protein (N^{Phi22}) and boxBPhi21, Phi 22 N protein (N^{Phi22}) and boxBP22, lambda N protein (N^{lambda}) and boxBlambda, an evolved RNA-binding HUH endonuclease⁹, or Com and com. In some embodiments, the clkDNA tethering domain is fused to the DNA binding domain on the N terminus or the C terminus, or is inlaid internally into the DNA binding domain. In some embodiments, the clkNA tethering domain is fused to the effector domain, and where the DNA binding domain is separate.

Further, provided herein are click editor compositions comprising a clkDNA tethering domain and a DNA binding domain in a non-covalent complex formed by interaction of protein recruitment domains on each of the clkDNA tethering domain and the DNA binding domain, and optionally an effector domain, optionally wherein the effector domain is separate from both the clkDNA tethering domain and the DNA binding domain. In some embodiments, the protein recruitment domains are interacting coiled coil, leucine zipper, or Suntag-scFv domain pairs (i.e., the interacting pairs bind to each other).

Also provided herein are click editor compositions comprising a DNA binding domain, a clkDNA tethering domain, and an effector domain, optionally wherein the clkDNA tethering domain and the DNA binding domain are in a single fusion protein, and the effector domain is separate.

In some embodiments, the DNA binding domain (e.g. an RNA-programmable nickase or nuclease) is a non-target strand nickase or a nuclease (e.g., Cas9 nickase or nuclease, Cas12 nickase or nuclease, an IscB nickase or nuclease, a TnpB nickase or nuclease, etc.; see Table C).

In some embodiments, the clkDNA tethering domain is an HUH endonuclease, avidin, SNAP-tag, CLIP-tag, or a HALO-tag. In some embodiments, the clkNA tethering domain is an RNA binding protein, e.g., a phage coat protein (CP), e.g., MCP, PCP, or Com; or an evolved RNA-binding HUH endonuclease⁹.

In some embodiments, the effector domain is a DNA polymerase or ligase.

In some embodiments, the DNA polymerase is a DNA-dependent DNA polymerase of family A, B, C, D, X, or Y, optionally E. coli Klenow (EcKlenow, optionally with the D355A and/or E357A mutations that deactivate its 3′-5′ exonuclease domain); Taq Stoffel; Pol-Beta; Pol-Beta+Sso7d; Phi29 DNA Polymerase (D169A); Sequenase; T4 DNA Polymerase, and E. coli dKlenow (optionally with the D355A, E357A, D705A, and/or D882A mutations), or is a reverse transcriptase, optionally MarathonRT, GsI-IIC RT, and MMLV-RT variants, e.g., PE2 MMLV RT (with D200N, T306K, W313F, T330P, L603W mutations), or MMLV or PE2 MMLV RT truncations (e.g., truncations 2, 5, or 6), MMLV variants encoded in PE6a-PE6g¹⁰, (e.g. or M-MLV RT (T128N, V223Y, D200C, with or without RNaseH domain truncation of M-MLV), Gs RT or Gs RT (A16E, L37P, A123V), Tfl RT or Tfl RT (P70T, G72V, S87G, M102I, K106R, K118R, I128V, L158Q, F269L, A363V, K413E, S492N) or Tfl RT (P70T, G72V, S87G, M102I, K106R, K118R, I128V, L158Q, F269L, A363V, K413E, S492N, K118R, S188K, I260L, S297Q, R288Q), Ec48 RT or Ec48 RT (E60K, K87E, E165D, D243N, R267I, E279K, K318E, K343N), KORV RT, WMSV RT, AVIRE RT, BAEMV RT, PERV RT, MMTV RT, Ty3 RT, GALV RT, SRV2 RT, MPMV RT, Vp96 RT, ASLV RT), RT HFV, HERV, LtrA, HERV-Kcon, Tel4c, GsI-IIC, Ma-Int5, or engineered Marathon (optionally with D14R, N26R, D74R, Ni16K, or N197R mutations).

In some embodiments, the DNA Ligase is T3; T4; T7; ChlV (SplintR); PhiKMV; Vaccinia; or dT4 ligase.

In some embodiments, the click editor fusion proteins, compositions, and click editor compositions as described herein further comprise a recombinase fused to the complex or fusion protein comprising the DBD, recruited to the DBD by a protein recruitment domain, or expressed separately in trans.

Additionally provided herein are methods of altering a target DNA sequence, e.g., a genomic sequence, using the click editor fusion proteins, compositions, and click editor compositions as described herein. In some embodiments, the methods comprise comprising contacting the DNA sequence with: (i) a DNA binding domain (e.g. an RNA-programmable DNA nickase or nuclease), a clkDNA tethering domain, and an effector domain, optionally in one or more fusion proteins, e.g., in a click editor fusion proteins, compositions, or click editor compositions described herein; (ii) a clkDNA template as described herein, e.g., wherein the localization moiety of the clkDNA template binds to the clkDNA tethering domain of the click editor fusion protein; and (iii) a guide RNA that directs the RNA-programmable DNA nickase to the target DNA sequence.

Also provided herein are methods of altering a target DNA sequence, e.g., deletion, replacement, or duplication of the target DNA sequence, the method comprising contacting the DNA sequence with: (i) a DNA binding domain (e.g. an RNA-programmable nickase or nuclease), a clkDNA tethering domain, and an effector domain, optionally in one or more fusion proteins, e.g., in a click editor fusion protein, composition, or click editor composition as described herein; (ii) the clkDNA template as described herein, e.g., wherein the localization moiety of the clkDNA template binds to the clkDNA tethering domain of the click editor fusion protein; and (iii) a pair of sgRNAs, each targeting opposite DNA strands, to generate two 3′ flaps that are either complementary to upstream sequence (relative to the nick site) or to each other (and sufficiently dissimilar to the DNA between the two nicks).

Further, provided herein are methods of altering a target DNA sequence, e.g., a genomic sequence. The methods include contacting the DNA sequence with: (i) a DNA binding domain (e.g., an RNA-programmable DNA nickase or nuclease) linked to a clkDNA tethering domain with optional linkers therebetween, (ii) a clkDNA template as described herein, e.g., wherein the localization moiety of the clkDNA template binds to the clkDNA tethering domain; (iii) an effector domain linked to an RNA binding protein, e.g., MCP, PCP, or Com RNA binding protein;
and (iii) one or a pair of sgRNAs, each targeting opposite DNA strands, to generate two 3′ flaps that are either complementary to upstream sequence (relative to the nick site) or to each other (and sufficiently dissimilar to the DNA between the two nicks), wherein one or both of the sgRNAs comprises a MS2, PP7, or com RNA sequence.

Additionally, provided herein are methods for altering a target DNA sequence, e.g., a genomic sequence. The methods comprises contacting the DNA sequence with: (i) a DNA binding domain (e.g., an RNA-programmable DNA nickase or nuclease), a clkNA tethering domain, and an effector domain, optionally in one or more fusion proteins, e.g., in a click editor fusion protein, composition, or click editor composition as described herein; (ii) a clkNA template as described herein, comprising a localization moiety, a polymerization template (PT) comprising at least one desired edit or an attachment duplex region (ADR) comprising at least one desired edit, a first primer binding site (PBS1), and an extended 3′ DNA flap comprising the reverse complement of a second PBS (rcPBS2), wherein the localization moiety of the clkNA template binds to the clkNA tethering domain of the click editor fusion protein; and (iii) a pair of guide RNAs that direct the RNA-programmable DNA nickase to the target DNA sequence, wherein: the RNA-programmable DNA nickase nicks the non-target strand at a first nick site on the target site directed by a first gRNA; the PBS1 of the clkNA template anneals to the non-target DNA strand; an extended 3′ DNA flap comprising a sequence complementary to PBS2 is generated by the effector domain, the extended 3′ DNA flap comprising PBS2 anneals to the 3′ flap at a second nick site directed by a second gRNA; the effector domain carries out second strand synthesis; and the newly synthesized DNA is incorporated into the target DNA, leading to altering the target DNA.

Also provided are the clkDNA templates or composition described herein, wherein the edit comprises insertion of an attP or attB sequence.

In some embodiments, the edit comprises insertion of an attP or attB sequence, and the method further comprises contacting the DNA with a donor template comprising attR and attL sequences, and a serine recombinase, optionally BxBl or Pa01, optionally fused to the click editor fusion protein.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting.

All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A-C. Polymerase Click Editor (PCE) components and mechanism. a, Generic schematic of an HUH endonuclease mechanism. The HUH endonuclease forms a phosphotyrosine adduct with a single-stranded DNA (ssDNA) containing an HUH recognition sequence. b, PCE1 components and mechanism—which uses target-primed, DNA-templated polymerization of an edit of interest contained on an HUH-endonuclease-localized clkDNA. In this example, the clkDNA includes an HUH endonuclease recognition sequence (HUH site), a polymerization template (PT), and a flap binding region (FBR) (which is complementary to the genomic flap released by nCas9). Flap equilibration between the newly synthesized 3′-flap and the 5′-endogenous flap, followed by DNA repair, incorporates or rejects the edit contained in the clkDNA. c, PCE2 (bottom row) and PCE2b (top row) components and mechanism. This method uses a second nicking guide to bias DNA mismatch repair to incorporate, instead of reject, the edit of interest.

FIGS. 2A-D. PCE click DNA (clkDNA) titration and Cas9 activity-enhancing mutations comparison. a, clkDNA titration (0-32 pmols) with PCE2, showing percentage of reads containing the intended AGG deletion (left) and indels (right) when targeting a site in the DNMT1 gene. b, Editing efficiency comparison of nCas9 only and PCE2 at the same DNMT1 locus, when using 16pmol of clkDNA. c, clkDNA titration (0-32 pmols) with PCE2, showing percentage of reads containing the intended AGC insertion (left) and indels (right) when targeting a site in the NOLC1 gene. dPCV2 denotes a catalytically inactive PCV2. d, Comparison of editing efficiencies for PCE2 containing nCas9 with or without R221K and N394K mutations, previously shown to modestly increase Cas9 nuclease activity¹⁸. 2 pmols or 4 pmols of clkDNA were used. Mean, s.d., and individual datapoints shown for n=3 technical replicates for all.

FIGS. 3A-D. PCE1 and PCE2 comparison, PCE2 with controls, and PCE2 characterization at various genomic sites. a, b, Comparison of PCE1 and PCE2 constructs at endogenous human genomic loci including DNMT1 (a) and RNF2 (b). PCE1 contains only the primary gRNA, PCE2 contains an additional gRNA to create a secondary nick to favor incorporation of the edit, and PCE2s contains an additional gRNA that overlaps the edit so is less likely to nick the unedited allele. c, Controls to assess the importance of each PCE/clkDNA component for precise editing or indels. nCas9, nickase Cas9; dCas9, dead Cas9; dEcKlenow, dead EcKlenow. d, PCE2 editing efficiencies and indels at a range of genomic sites using various clkDNAs to install diverse edits. Mean, s.d., and individual datapoints shown for n=3 technical replicates for all.

FIGS. 4A-D. Screening different HUH endonucleases and DNA polymerases in a PCE2 architecture. a,b, Screen to assess the efficiencies of various HUH endonucleases fused to the N-terminal end of the nCas9-EcKlenow construct, when targeted to sites in DNMT1 (a) or RNF2 (b). c,d, Screen to assess the efficiencies of DNA polymerase fused to the C-terminal end of the PCV2-nCas9 construct, when targeted to sites in DNMT1 (c) or RNF2 (d). Mean, s.d., and individual datapoints shown for n=3 technical replicates for all.

FIGS. 5A-B. PCEs for targeted, in cellulo diversification of genomic DNA sequences. a, Strategy for PCE-mediated targeted diversification, using mixed oligos or oligo pools. b, (SEQ ID NO: 574) Editing efficiency and library representation when using PCE2 with an oligo pool targeting FANCF. Each oligo in the pool contained a protospacer adjacent motif (PAM) edit 5 bp away from the nick site (+5 G to T) and another edit at one of four downstream bases from the PAM edit. The average of n=3 technical replicates is shown for 5B.

FIGS. 6A-B. Dual-flap PCE (“Double Click”) strategies and editing types. Different clkDNA designs and respective strategies (1-5) to link a given sgRNA and a corresponding clkDNA FBR-PT. This approach enables dual-flap editing resulting in precise deletions (a), precise replacements (b), and precise duplications of DNA sequences (c).

FIGS. 7A-C. Ligase Click Editor (LCE) strategy. a, LCE components and mechanism—which uses ligation of an attachment sequence located on a clkDNA. The clkDNA consists of two annealed oligos: (1) an oligo that contains an HUH endonuclease recognition sequence (HUH site), an attachment duplex region (ADR), and a flap binding region (FBR) (which is complementary to the genomic flap released by nCas9). The FBR and ADR combined are designated ‘splint’ (2) an attachment oligo containing the edit of interest which anneals to the ADR of the first oligo. Splint ligation of the attachment on the genomic flap, flap equilibration between the newly attached 3′-flap and the endogenous 5′-flap, followed by DNA repair, incorporates or rejects the edit contained in the attachment. b, LCE2 and LCE2b mechanisms and components—which uses recruitment of the ligase domain only to the first spacer target (e.g., through MS2-MCP interaction) and a second nicking guide to nick the non-edited strand, biasing mismatch repair to incorporate, instead of reject, the edit of interest. c, LCE2 editing efficiency when targeting FANCF (+5 G to T) and using an sgRNA that directs Cas9-nicking +48 bp of the edit site. Mean, s.d., and individual datapoints shown for n=3 technical replicates for all.

FIGS. 8A-F. Dual-overhang ligation mechanism for targeted DNA replacement. a, Dual-overhang ligation components. b, Dual-overhang ligation mechanism. c, Dual-overhang ligation mechanism using a gapped clkDNA. d, Replacement efficiencies, indels, and purity of a 48 bp replacement with 48 bp of orthogonal sequence at FANCF. T4: T4 DNA polymerase; dT4: catalytically inactive T4 DNA polymerase e, Localization strategy using 3′ biotin-labeled clkDNAs and a monomeric avidin fusion to nCas9-Ligase. f, Replacement efficiencies, indels (left), and purity (right) of a 48 bp replacement with 48 bp of orthogonal sequence at FANCF. Mean, s.d., and individual datapoints shown for n=3 technical replicates for all.

FIG. 9. Programmable gene-sized DNA insertions with PCEs, LCEs, or dual-overhang ligation combined with serine recombinases. PCEs, LCEs, or dual-overhang ligation approaches can be used to install a recombinase attachment site (att) at a desired position in the genome. Serine recombinases, either fused to PCE/LCE or expressed in trans, integrate a DNA donor containing a corresponding recombinase attachment site and a cargo of interest at the targeted location.

FIGS. 10A-G. Exemplary configurations of click editor protein components. a, Schematic of click editor components. b-g, exemplary configurations. Click editors can be configured in various ways including as unfused components (as shown in b), tripartite fusions with the DNA nickase in the center (as shown in c), tripartite fusions with the DNA nickase on the C-terminus (as shown in d), tripartite fusions with the DNA nickase on the N-terminus (as shown in e), bipartite fusions with separate expression of the third domain (as shown in f; i.e., the DNA nickase and clkDNA tethering domain fused with an unfused effector domain, bipartite fusion of the DNA nickase and effector domain domain in combination with an unfused clkDNA tethering domain, or bipartite fusion of the clkDNA tethering domain and the effector domain in combination with an unfused DNA nickase), inlaid compositions (as shown in g), or other configurations.

FIGS. 11A-B. Exemplary configurations of clkDNA templates. Schematics of click DNA (clkDNA) composition for polymerase click editors (PCEs; as shown in 11a) and ligase click editors (LCEs; as shown in 11b).

FIGS. 12A-L. Overview and development of click editing. a, Schematic of a click editor (CE), which is a fusion protein consisting of an RNA-programmed DNA nickase, a DNA-dependent DNA polymerase, and an HUH endonuclease (HUHe) paired with a guide RNA (gRNA). The click-DNA (clkDNA) template is a single-stranded DNA oligonucleotide that encodes a primer binding site (PBS), a polymerase template (PT), and an HUHe recognition site b, Phylogenetic tree generated from 580 sequences¹⁹ (Table 6) depicting a small subset of HUHe diversity across domains of life. Scale represents the fractional distance relatedness between sequences. c, Schematic of an HUHe forming a covalent phosphotyrosine adduct with a ssDNA molecule, where the HUHe binds a recognition sequence to initiate a click-like conjugation reaction. d, Stepwise click editing mechanism involving: (1) a DNA target site nick to release the non-target strand (NTS) 3′-genomic flap, (2) NTS flap hybridization with the clkDNA PBS, (3) NTS-PBS junction to prime synthesis by the DNA-dependent DNA polymerase, (4) extension of the 3′ NTS flap to polymerize from the edit-encoding PT of the clkDNA, (5) equilibration between the newly synthesized 3′ and native genomic 5′ flaps, and (6) 5′-flap cleavage leading to edit incorporation. e, Schematic of click editing transfections in HEK 293T cells, involving co-transfection of a CE plasmid (porcine circovirus 2 (PCV2) HUHe fused to nSpCas9(H840A) and Klenow fragment from E. coli DNA polymerase I (D355A, D357A) (EcKlenow)), a clkDNA, and one (or two) gRNA plasmid(s). Editing efficiency is assessed 72 hours post-transfection following genomic DNA extraction and amplicon sequencing. f,g, Percentage of sequencing reads with precise edits or insertion or deletion mutations (indels) using the DNMT1 gRNA and a clkDNA with PBS13-PT12 encoding a +3-5 AGG deletion (with a +49 nick; f), or the RNF2 gRNA and a clkDNA with PBS15-PT14 encoding a +4 A-to-C substitution (with a +5 ‘2b’ nick; g). CE1, CE (PCV2-nSpCas9 (H840A)-EcKlenow) with one gRNA to direct non-target strand nicking; CE1.n2, CE1 with an additional gRNA to direct nicking (i.e. ngRNA) targeted against the non-edited strand at a specified distance from the nick generated by the primary gRNA; CE1.n2b, CE1 with a ngRNA that binds only to the edited strand, directing nicking to the unedited strand; nCas9, CE1.n2 with nCas9 (no HUHe or DNA pol.) and a clkDNA lacking the HUHe recognition site; dCas9, CE1.n2 with a catalytically-deactivated Cas9 (dCas9; D10A, H840A) fused to PCV2 and EcKlenow; dPCV2, CE1.n2 with a catalytically inactive PCV2 (Y96F) fused to an nCas9 and EcKlenow; dKlenow, CE1.n2 with a catalytically inactive EcKlenow (D355A, D357A, D705A, D882A) fused to nCas9 and PCV2. Data in f and g from HEK 293T cell experiments; mean, s.d., and individual datapoints shown for n=3 independent biological replicates. h, Representative structure of the PCV2 HUHe (grey) bound to a ssDNA substrate (orange) (PDB ID: 6WDZ). i,j, Percentage of sequencing reads with precise edits when using CE constructs encoding different HUHe domains to install edits using the DNMT1 or RNF2 gRNAs (i and j, respectively). DCV, duck circovirus; MSMV, maize striate mosaic virus; TraI, E. coli conjugation protein TraI; RepBm, RepB Fructobacillus tropaeola; FBYNV, fava bean necrosis yellow virus; TGMV, tomato golden mosaic virus. k,l, Percentage of sequencing reads with precise edits when using CE constructs encoding different DNA-dependent DNA polymerases installing edits using the DNMT1 or RNF2 gRNAs (k and 1, respectively). EcKlenow, Klenow fragment from E. coli DNA polymerase I (D355A, D357A); TaqStoffel, Stoffel fragment from Thermus aquaticus DNA polymerase; M-MLV RT, engineered pentamutant Moloney Murine Leukemia Virus reverse transcriptase from PE2 (Anzalone et al., Nature. 2019 December; 576(7785):149-157); Polp, human polymerase beta; Phi29, DNA polymerase from bacteriophage φ29 (D169A); Sequenase, engineered truncation of T7 bacteriophage DNA polymerase; T4, T4 bacteriophage DNA polymerase. Data in i-1 from HEK 293T cell experiments; mean, s.d., and individual datapoints shown for n=3 independent technical replicates.

FIGS. 13A-O. Optimization of clkDNA parameters. a, Schematic of clkDNA screens in 96-well format. The CE, gRNA and ngRNA (CE1.n2) are transfected together with up to 96 unprotected clkDNA oligonucleotides (oligos) with various PBS and PT lengths arrayed on a plate. Optimal clkDNA candidates can then be further chemically modified (e.g., with two phosphorothioate (PS) linkages) for validation studies. b, Percentage of sequencing reads with a precise +3-5 AGG deletion using the DNMT1 gRNA, in a clkDNA screen using unmodified oligos to vary the PBS and PT lengths. c, Percentage of sequencing reads with precise edits or indels, when assessing the most efficient DNMT1 clkDNAs but with 2×3′-PS linkages on the clkDNA. d, Percentage of sequencing reads with a precise +5 G-to-C transversion using the ACTB gRNA, in a clkDNA screen using unmodified oligos to vary the PBS and PT lengths. e, Percentage of sequencing reads with precise edits or indels, when assessing the most efficient ACTB clkDNAs but with 2×3′-PS linkages on the clkDNA. f, Percentage of sequencing reads with precise edits or indels with different nicking gRNAs (ngRNA) targeting ACTB and a 2×3′-PS protected clkDNA of PBS16-PT19. g, Percentage of sequencing reads with precise edits or indels, when assessing the most efficient ACTB clkDNAs with 2×3′-PS linkages on the clkDNA and a 2b ngRNA (n2b, +5). h, Percentage of sequencing reads with a precise +4 AT insertion using the TGFBI gRNA, in a clkDNA screen using unmodified oligos to vary the PBS and PT lengths. i, Percentage of sequencing reads with precise edits or indels, when assessing the most efficient TGFBI clkDNAs but with 2×3′-PS linkages on the clkDNA. j, Percentage of sequencing reads with a precise dual +1 T-to-A & +5 G-to-C edit using the IL2RB gRNA, in a clkDNA screen using unmodified oligos to vary the PBS and PT lengths. k, Percentage of sequencing reads with precise edits or indels, when assessing the most efficient IL2RB clkDNAs but with 2×3′-PS linkages on the clkDNA. 1, Percentage of sequencing reads with a precise +6 G-to-T edit using the PRNP gRNA, in a clkDNA screen using unmodified oligos to vary the PBS and PT lengths. m, Percentage of sequencing reads with precise edits or indels, when assessing the most efficient PRNP clkDNAs but with 2×3′-PS linkages on the clkDNA. n, Percentage of sequencing reads with a precise +2 G deletion using the GJB2 gRNA, in a clkDNA screen using unmodified oligos to vary the PBS and PT lengths. o, Percentage of sequencing reads with precise edits or indels, when assessing the most efficient GJB2 clkDNAs but with 2×3′-PS linkages on the clkDNA. Data in b,d,h,j,l,n from HEK 293T cell experiments; mean, s.d., and individual datapoints shown for n=3 independent biological replicates. Data in c,e,f,g,i,k,m,o from HEK 293T cell experiments; mean, s.d., and individual datapoints shown for n=3 independent technical replicates.

FIGS. 14A-F: DNA repair evasion through clkDNA modification. a, Schematic of DNA repair engagement on substrates with different compositions of mismatches. The MutS/MutL mismatch repair (MMR) complex, or other mechanisms (e.g., DNA repair or otherwise), can excise the DNA flap encoding the intended edit (1° edit, teal); encoding additional substitutions (2° mismatch, yellow) adjacent to the intended edit (1° edit, teal) may evade excision of the intended edit. b, (SEQ ID NO(s): 581-586) Percentage of sequencing reads with precise +1 T-to-A and +5 G-to-C transversions using CE1.n1 or CE1.n2, the IL2RB gRNA, and clkDNAs encoding additional mutations for MMR evasion. Colors represent nucleotide changes. Ref:, reference amplicon; triangle, gRNA nick site; PAM, protospacer adjacent motif. c, (SEQ ID NO(s): 575-580) Percentage of sequencing reads with a precise +5 G-to-T transversion using CE1.n1, the VEGFA gRNA, and clkDNAs encoding additional mutations for MMR evasion. d, (SEQ ID NO(s): 587-594) Percentage of sequencing reads with a precise +5 G-to-C transversion using CE1.n1, the ACTB gRNA, and clkDNAs encoding additional mutations for MMR evasion. e, (SEQ ID NO(s): 595-659) Percentage of sequencing reads with a precise +5 G-to-C transversion using CE1.n1, the ACTB gRNA, and clkDNAs encoding all possible bases in three positions of the clkDNA for MMR evasion. f, (SEQ ID NO: 595) Violin plots depicting percentage of reads with precise edits in ACTB depending on the nature and the position of the mutation within the clkDNA. The query base is shown with a box and ‘Substituted to. −’ depicts a clkDNA without additional mismatches. Data in c,d,e,f from HEK 293T cell experiments; mean, s.d., and individual datapoints shown for n=3 independent biological replicates. Data in b from HEK 293T cell experiments; mean, s.d., and individual datapoints shown for n=3 independent technical replicates.

FIGS. 15A-P: Comparison to prime editing, off-target analyses, and architectural alterations a-c, Percentage of sequencing reads with precise edits or indels using CE1 (PCV2-nSpCas9(H840a)-EcKlenow), PE1 (nSpCas9(H840A)-M-MLV-RT), PE2 (nSpCas9(H840A)-M-MLV-RT(D200N/L603W/T330P/T306K/W313F) from Anzalone et al. Nature, 2019), or PE3 (PE2+ngRNA) when targeting VEGFA (with CE1.n1, no ngRNA for CEs or PEs; a), DNMT1 (with CE1.n2(+49), using the +49 ngRNA for CEs and PEs; b), or ACTB (with CE1.n2b(+5), using the +5 ngRNA for CEs and PEs; c). For CEs, clkDNAs were optimized in this study; for PEs, pegRNAs were previously optimized for VEGFA and DNMT1 (from Anzalone et al. Nature, 2019), and we performed a small optimization of pegRNAs for ACTB. WT, wild-type. d, Proportion of modified reads containing template-mediated insertions. For CE1 edits, mutations were detected that matched the clkDNA template including the 4nt linker between the HUHe site and PT on the clkDNA, or that harbored insertions templated only from the HUHe site. For PE1 and PE2, insertions corresponding to the sgRNA scaffold insertions are reported. e,f Percentage of reads in experiments using the VEGFA gRNA with precise editing or indels at the on-target site (e) or off-target sites (f) using CE1.n1 or SpCas9 nuclease compared to an untransfected control. g,h, Percentage of reads in experiments using the DNMT1 gRNA with precise editing or indels at the on-target site (g) or off-target sites (h) using CE1.n2(+49) or SpCas9 nuclease compared to an untransfected control. i, Ratio of off-target to on-target editing for selected off-target sites in VEGFA and DNMT1, using CE1.n1 or CE1.n2, respectively, or SpCas9 (data from f and h). j, Schematic of possible HUHe-dependent interaction with genomic sites containing an HUHe recognition sequence that are transiently ssDNA during cellular replication or transcription. k, Percentage of sequencing reads with precise edits for DNMT1, RNF2 and ACTB (on-target editing) from experiments with various CE1 conditions. 1, Percentage of sequencing reads with indels at PCV2 HUHe pseudosites in the human genome in various CE1 conditions targeting either DNMT1, RNF2 or ACTB. m, Schematic of different CE1 architectures tested. CC, coiled-coil domains N5/N6^{21,22}; EcKlenow, Klenow fragment from E. coli DNA polymerase I (D355A, D357A); Phi29, DNA polymerase from bacteriophage φ29; Phi29 (D169A), 3′-5′ exonuclease-deficient Phi29 DNA polymerase; ePhi29, engineered thermostable Phi29 DNA polymerase (M8R, V51A, M97T, G197D, E221K, Q497P, K512E, F526L); ePhi29 (D169), 3′-5′ exonuclease-deficient ePhi29 (D169A, M8R, V51A, M97T, G197D, E221K, Q497P, K512E, F526L); eB103, engineered thermostable Phi29 ortholog (H73R, A147K, R221Y, A318G, M339L, E359D, K372E, F383L, D384N, A503M, I511V, R544K, T550K. n-p, Percentage of sequencing reads with edits in ACTB, PRNP and DNMT1 when using CE1.n2 constructs encoding different DNA-dependent polymerases and different construct architectures. Data in a-i,k,l from HEK 293T cell experiments; mean, s.d., and individual datapoints shown for n=3 independent biological replicates. Data in n-p from HEK 293T cell experiments; mean, s.d., and individual datapoints shown for n=3 independent technical replicates.

FIG. 16: Schematics of clkNA configurations comprised of various modified or unmodified DNA or RNA bases. The clkNA optionally harboring various regions including a localization sequence, a polymerization template (PT), and/or a primer binding site (PBS) can be fully or partly comprised of different nucleic acid compositions at any position (DNA, RNA, modified bases, unmodied bases, etc.).

FIGS. 17 A-F: Click Editing efficiency with different Cas9 orthologs, different human cellular models and mRNA delivery. a, Percentage of sequencing reads with a precise +6 G-to-C edit or insertions and deletions (indels) in experiments targeting the EMX1 locus using a Staphylococcus aureus Cas9 (SaCas9)-based CE1 construct (PCV2-nSaCas9(N580A)-EcKlenow), comparing two ngRNAs (+60 and +69), and 2×3′-PS protected clkDNAs with varying PBS and PT lengths. b, Percentage of sequencing reads with a precise 3 nt substitution or indels when targeting the ACTB locus (PBS16-PT19) using a CE1 construct expressing EcKlenow or ePhi29(D169) in different human cell lines (U2OS, HeLa, HCT116 and HEK 293). c, Percentage of sequencing reads with a precise 3nt substitution edit or indels when targeting the ACTB locus in HeLa cells using a CE1 construct expressing EcKlenow or ePhi29(D169), and 2×3′-PS or 3×3′-PS/2′-O-Methyl RNA (2′-O-Me) clkDNAs (PBS16-PT19). d, Schematic of transfection of HEK 293T, HeLa and HCT116 cells with CE1 mRNA, clkDNA and synthetic sgRNAs (spacer and ngRNA). e, Percentage of sequencing reads with a precise +5 G-to-C edit or indels in the ACTB locus (PBS16-PT19) via CE mRNA delivery (d) in HEK 293T, HeLa and HCT116 cells. f, Potential future optimizations for engineering improved CEs. Data in a, from HEK 293T cell experiments; mean, s.d., and individual datapoints shown for n=3 independent biological replicates. For the data in b,c,e, the mean, s.d., and individual datapoints are shown for n=3 independent technical replicates.

FIGS. 18A-D: Assessment of HUHe orthologs for clkDNA recruitment. a,b, Percentage of sequencing reads with precise edits or insertion or deletion mutations (indels) when using CE constructs encoding different HUHe domains to install edits at DNMT1 (with a PBS13-PT 12 clkDNA) or RNF2 (with a PBS15-PT 14 clkDNA) (a and b, respectively). PCV2, porcine circovirus 2; DCV, duck circovirus; MSMV, maize striate mosaic virus; TraI, E. coli conjugation protein TraI; RepBm, RepB Fructobacillus tropaeola; FBYNV, fava bean necrosis yellow virus; TGMV, tomato golden mosaic virus. c, Phylogenetic tree of circovirus and cyclovirus HUHes tested in this study, constructed with Geneious (v2024.0.2) using “global alignment with free end gaps” and “Blosum 62” cost matrix settings. d, Percentage of sequencing reads with precise edits or indels when using CE1 constructs encoding different HUHe domains from the circovirus and the cyclovirus families to edit the DNMT1 locus at varying clkDNA doses (PBS16-PT10). PCV1, porcine circovirus 1; BDFV, Beak and feather disease virus; CaCV, Canary circovirus; CoCV, Columbid circovirus; FiCV, Finch circovirus; GoCV, Goose circovirus; GuCV, Gull circovirus; RaCV, Raven circovirus; StCV, Starling circovirus; SwCV, Cygnus olor circovirus; Chimp-Chimpanzee Stool avian-like circovirus; NG, Nigeria; PK, Pakistan; T, Tunisia. Data in a, b, and d from HEK 293T cell experiments. mean, s.d., and individual datapoints shown for n=3 technical replicates.

FIGS. 19A-C: Template recruitment via a telomere binding protein (TBP). a, Schematic of the mechanism of action of the yeast TBP protein, Cdc13, which recognizes and binds to a consensus sequence in the ssDNA portion of telomeric regions. b, Schematics of different CE1 architectures and clkDNAs/templates tested, including a representative literature analysis of the approximate substrate binding affinities measured for DCV HUHe (Smiley et al., mBio. 2023 Feb. 28; 14(1):e0258722)(and by analogy PCV2 HUHe), Cdc13 TBP (Chandra et al., Genes Dev. 2001 Feb. 15; 15(4):404-14), and the Cdc13(Y556A) (Glustrom et al., Proc Natl Acad Sci USA. 2018 Oct. 9; 115(41):10315-10320) enzyme variant (right panel). c, Percentage of sequencing reads with precise edits or insertion or deletions mutations (indels) when using CE1 constructs encoding PCV2, Cdc13 or Cdc13(Y556A) or only nCas9 to edit the DNMT1 locus at varying clkDNA doses (PBS16-PT10). Datapoints in c from HEK 293T cell experiments; mean, s.d., and individual datapoints shown for n=3 technical replicates.

FIGS. 20A-C: clkDNA recruitment by mSA-biotin. a, Schematic of different CE1 architectures and clkDNA templates. mSA, monomeric streptavidin. b,c, Percentage of sequencing reads with precise edits when targeting the ACTB and the DNMT1 loci (b and c, respectively) when using a CE comprised of PCV2 (in orange) or mSA (in blue, combined with a 5′biotin clkDNA) for clkDNA recruitment, as well as EcKlenow or an engineered thermostable Phi29 DNA polymerase (ePhi29; Povilaitis et al., Protein Eng Des Sel. 2016 December; 29(12):617-628). Data in b,c from HEK 293T cell experiments; mean, s.d., and individual datapoints shown for n=3 technical replicates.

FIGS. 21A-E: clkDNA recruitment by MCP-MS2. a, (SEQ ID NO(s): 391, 547 and 546) Schematic of different CE1 architectures and clkDNA templates. MS2 clkDNAs can have 3×2′oME RNA bases on the 5′ end (‘MS2 & 2′oME clkDNA’) or have an unprotected architecture (‘MS2 clkDNA’) MCP, MS2-coat protein. b, Percentage of sequencing reads with precise edits when targeting the ACTB and the DNMT1 loci when using a CE comprised of PCV2 or MCP (N55K) for clkDNA recruitment, as well as EcKlenow, Phi29 or an engineered thermostable Phi29 DNA polymerase (ePhi29; (Povilaitis et al., 2016, supra). c, Percentage of sequencing reads with precise edits when targeting the ACTB and the DNMT1 loci when using a CE comprised of MCP wild-type or MCP (N55K) for clkDNA recruitment and EcKlenow as a DNA-dependent DNA polymerase. MS2 clkDNA was used for this data. d, Schematics of CE1 unfused architecture, using MCP for protein recruitment and an MS2 clkDNA. e, Percentage of sequencing reads with precise edits when targeting the ACTB and the DNMT1 loci when using a CE comprised of MCP (N55K) for clkDNA recruitment, as well as EcKlenow, Phi29 or an engineered thermostable Phi29 DNA polymerase (ePhi29; Povilaitis et al., 2016, supra) in an unfused architecture. Data in b,c and e from HEK 293T cell experiments; mean, s.d., and individual datapoints shown for n=3 technical replicates.

FIGS. 22A-I: Click editing in different cell models, via mRNA delivery, and for longer edits. a,b, Percentage of sequencing reads with precise edits or insertion or deletion mutations (indels) when targeting the ACTB or DNMT1 loci (a and b, respectively) using a CE1 construct expressing EcKlenow or ePhi29(D169) in different human cell lines (U2OS, HeLa, HCT116 and HEK 293). c,d. Percentage of sequencing reads with precise edits or indels when targeting the ACTB and DNMT1 loci (c and d, respectively) in HeLa cells when using and 2×3′-PS or 3×3′-PS/2′-O-Methyl RNA (2′-O-Me) modified clkDNAs and CE1 constructs comprised of EcKlenow or engineered thermostable Phi29 DNA polymerase (ePhi29(D169A); Povilaitis et al., 2016, supra). e, Schematic of click editing in human primary fibroblasts. Fibroblasts were first transduced with lentiviral vectors to stably express the CE1 construct (PCV2-nCas9-EcKlenow), followed by puromycin selection to enrich for CE1-transduced cells, and then subsequent nucleofection with clkDNAs and gRNA-expression plasmids. Editing efficiencies were analyzed from unsorted populations of cells following nucleofection. f,g, Percentage of sequencing reads with precise edits or indels when targeting the ACTB locus for installing either a 3 nt substitution (+5 G-to-C, +6 G-to-C, +1 C-to-A) or a +5 G-to-C substitution (f and g, respectively) using CE1 and 2×3′-PS or 3×3′-PS/2′-O-Methyl RNA (2′-O-Me) modified clkDNAs in human primary fibroblasts, following the experimental setup described in e. SpCas9 control nucleofections were performed with an SpCas9 nuclease expression plasmid and a gRNA expression plasmid. h, Percentage of sequencing reads with precise edits or indels when targeting the DNMT1 locus in HEK 293T, HeLa and HCT116 cells, upon delivery of synthetic gRNAs (+49 n2 ngRNA), clkDNA, and the CE1 mRNA (encoding PCV2-nCas9-EcKlenow). i, Percentage of sequencing reads with precise edits for installation of 6× His (SEQ ID NO: 164)(18 bp), FLAG (24 bp) or LoxP (40 bp) sequences when targeting the HEK3 site in HEK 293T cells, when using a CE expressing EcKlenow or ePhi29(D169A). For data in a,b,d,g, the mean, s.d., and individual datapoints are shown for n=3 independent biological replicates. For data in c,f,h,i, the mean, s.d., and individual datapoints are shown for n=3 technical replicates.

FIGS. 23A-B. Schematic of CE1.n2 and CE1.n2b compositions. a,b, The use of a secondary nicking gRNA (ngRNA) for CE1.n2 or CE1.n2b conditions (when the ngRNA is located distal from the edit or overlaps the edit as shown in a and b, respectively) can modify click editing efficiency and/or the level of insertion or deletion mutations (indels) observed. The n2 and n2b nicking conventions are similar to the PE3 and PE3b nicking approaches for PEs²⁰.

FIGS. 24A-D Usage of n2 and n2b ngRNAs and impact of clkDNA concentration on click editing efficiency. a,b Percentage of sequencing reads with precise edits or reads with insertion or deletion mutations (indels) using clkDNAs, CE1, and n2 or n2b ngRNAs to install edits in DNMT1 (with a PBS13-PT12 clkDNA) or RNF2 (with a PBS15-PT14 clkDNA) (a and b, respectively). c, Titration of clkDNA dose (0-32 μmol) for installing a +3-5 AGG deletion in the DNMT1 locus using CE1.n2 or only nSpCas9(H840A). d, Comparison of precise editing efficiency and insertion or deletion mutations (indels) for the DNMT1 site using CE1.n2 with 12 or 16 pmol of clkDNA. Data in a-d from HEK 293T cell experiments; mean, s.d., and individual datapoints shown for n=3 technical replicates.

FIGS. 25A-B. Assessment of mutations in the PCV2 recognition sequence. a, (SEQ ID NO(s): 41 and 694) Schematic of the PCV2 recognition sequence and the mechanism of click-like bioconjugation. The −4 and +1 positions relative to the nick are labelled, as well as the active-site tyrosine residue in PCV2 (Y96). b, (SEQ ID NO(s): 41, 368, 366, 369, 660-693) Percentage of sequencing reads with a precise +3-5 AGG deletion in the DNMT1 locus using CE1.n2, the DNMT1 gRNA and clkDNAs (PBS16-PT1O) encoding substitutions in the PCV2 binding sequence. Data in b from HEK 293T cell experiments; mean, s.d., and individual datapoints shown for n=3 technical replicates.

FIGS. 26A-E. Assessment of different DNA-dependent DNA polymerases (DDPs) for click editing. a,b, Percentage of sequencing reads with precise edits or insertion or deletion mutations (indels) when using CE1 constructs encoding different DDPs installing edits at DNMT1 (with a PBS13-PT12 clkDNA) or RNF2 (with a PBS15-PT14 clkDNA) (a and b, respectively). EcKlenow, Klenow fragment from E. coli DNA polymerase I. TaqStoffel, Stoffel fragment from Thermus aquaticus DNA polymerase; M-MLV RT, engineered Moloney murine leukemia virus reverse transcriptase pentamutant (D200N/L603W/T330P/T306K/W313F) from PE2 (Anzalone et al., Nature. 2019 December; 576(7785):149-157); Polp, human polymerase beta; Phi29, DNA polymerase from bacteriophage φ29 (D169A); Sequenase, engineered truncation of T7 bacteriophage. c,d, Percentage of sequencing reads with precise edits or indels when using CE1 constructs encoding the DDP EcKlenow, wild-type M-MLV RT (used in PE1; (Anzalone et al., Nature, 2019, supra);), or the engineered pentamutant M-MLV RT (D200N/L603W/T330P/T306K/W313F) (used in PE2; (Anzalone et al., Nature, 2019, supra);) to install edits at DNMT1 (with a PBS16-PT 10 clkDNA) or RNF2 (with a PBS15-PT14 clkDNA) (c and d, respectively). e, (SEQ ID NO: 707) Percentage of sequencing reads with precise edits or indels at the DNMT1 locus (PBS13-PT12 clkDNA) using CE constructs encoding different linker variants between PCV2 and nSpCas9(H840A), as well as different PCV2 C-terminal truncations. Data in a-e from HEK 293T cell experiments; mean, s.d., and individual datapoints shown for n=3 technical replicates.

FIGS. 27A-F. DNMT1 clkDNA screens and validation. a, Percentage of sequencing reads harboring indels at the DNMT1 locus, using CE1.n2 and clkDNAs with varying PBS and PT lengths to install a +3-5 AGG deletion. b, Ratio of precise editing to indels at the DNMT1 locus, using CE1.n2 and clkDNAs with varying PBS and PT lengths. c, Scatter plot depicting percentage of edit and indels at the DNMT1 locus for CE1.n2 and clkDNAs with varying PS and PT lengths. Highlighted are the clkDNAs that led to some of the highest levels of editing, which we selected for validation. d,e, Percentage of sequencing reads with precise edits or indels at the DNMT1 locus with selected clkDNAs which are either unprotected or 2×3′PS protected (d and e, respectively), using CE1.n2. f, Scatterplot comparing percentage of sequencing reads with precise edits for the DNMT1 locus when using unmodified clkDNAs or modified clkDNAs bearing 2×3′PS. Data in a-c from HEK 293T cell experiments; mean, s.d., and individual datapoints shown for n=3 independent biological replicates. Data in d-f from HEK 293T cell experiments; mean, s.d., and individual datapoints shown for n=3 technical replicates.

FIGS. 28A-I. ACTB clkDNA screens, validation and additional clkDNA end-modifications. a, Percentage of sequencing reads harboring indels at the ACTB locus, using CE1.n2 and clkDNAs with varying PBS and PT lengths to install a +5 G-to-C substitution. b, Ratio of precise editing to indels at the ACTB locus, using CE1.n2 and clkDNAs with varying PBS and PT lengths. c, Scatter plot depicting percentage of edit and indels at the ACTB locus for CE1.n2 and clkDNAs with varying PS and PT lengths.

Highlighted are the clkDNAs that led to some of the highest levels of editing, which we selected for validation. d, (SEQ ID NO(s): 391 and 486-487) Schematic of clkDNAs tested in e. PCV2 binding sequence is colored in yellow; linker sequence in grey; PT is underlined; PS linkages shown via an asterisk “*”, and substitutions in the PBS are highlighted in red. e, Percentage of sequencing reads with precise edits or insertion or deletion mutations (indels) in the ACTB locus using clkDNAs with the modifications described in d (PBS16-PT19). f, (SEQ ID NO(s): 410, 492-493 and 695) Schematic of clkDNAs tested in g. g, Percentage of sequencing reads with precise edits or indels in the DNMT1 locus using clkDNAs with the modifications described in f (PBS16-PT10). h, (SEQ ID NO(s): 391, 486, 696, 489 and 491) Schematic of clkDNAs tested in i, with modifications as described in e while also including 2′-O-Me, 2′-O-Methyl RNA (in blue); 3′ evopreQ₁, pseudoknot (Roth et al., Nat Struct Mol Biol. 2007 April; 14(4):308-17; Anzalone et al., Nat Methods. 2016 May; 13(5):453-8; Nelson et al., Nat Biotechnol. 2022 March; 40(3):402-410). i, Percentage of sequencing reads with precise edits or insertion or deletion mutations (indels) in the ACTB locus using clkDNAs with the modifications described in h (PBS16-PT19). Data in a-c from HEK 293T cell experiments; mean, s.d., and individual datapoints shown for n=3 independent biological replicates. Data in e,g,I from HEK 293T cell experiments; mean, s.d., and individual datapoints shown for n=3 technical replicates.

FIGS. 29A-B. Assessment of clkDNA templates with modified 3′ends. a, Schematic of the 3′-end modifications tested in b. Modifications include a 3′ hairpin with 2 nt tail (configuration ‘b’), a 3′ stem loop with 2 nt tail (configuration ‘c’), and the 3′ hairpin or 3′ stem without the 2 nt tail (configurations ‘d’ and ‘e’, respectively). b, Percentage of sequencing reads with precise edits when using clkDNAs encoding the modifications described in a to install edits using gRNAs targeted to ACTB (PBS16-PT19 clkDNA), DNMT1 (PBS16-PT 10 clkDNA) and RNF2 (PBS15-PT 14 clkDNA). Data in b from HEK 293T cell experiments; mean, s.d., and individual datapoints shown for n=3 technical replicates.

FIGS. 30A-D. TGFBI clkDNA screens and validation. a, Percentage of sequencing reads harboring indels at the TGFBI locus, using CE1.n2 and clkDNAs with varying PBS and PT lengths to install a +4 AT insertion. b, Ratio of precise editing to indels at the TGFBI locus, using CE1.n2 and clkDNAs with varying PBS and PT lengths. c, Scatter plot depicting percentage of edit and indels at the TGFBI locus for CE1.n2 and clkDNAs with varying PS and PT lengths. Highlighted are the clkDNAs that led to some of the highest levels of editing, which we selected for validation. d, Percentage of sequencing reads with precise edits or indels at the TGFBI locus with selected clkDNAs which are 2×3′PS protected, using CE1.n2. Data in a-c from HEK 293T cell experiments; mean, s.d., and individual datapoints shown for n=3 independent biological replicates. Data in d from HEK 293T cell experiments; mean, s.d., and individual datapoints shown for n=3 independent technical replicates.

FIGS. 31A-D. IL2RB clkDNA screens and validation. a, Percentage of sequencing reads harboring indels at the IL2RB locus, using CE1.n2 and clkDNAs with varying PBS and PT lengths to install a dual +1 T-to-A and +5 G-to-C edit. b, Ratio of precise editing to indels at the IL2RB locus, using CE1.n2 and clkDNAs with varying PBS and PT lengths. c, Scatter plot depicting percentage of edit and indels at the IL2RB locus for CE1.n2 and clkDNAs with varying PS and PT lengths. Highlighted are the clkDNAs that led to some of the highest levels of editing, which we selected for validation. d, Percentage of sequencing reads with precise edits or indels at the IL2RB locus with selected clkDNAs which are 2×3′PS protected, using CE1.n2. Data in a-c from HEK 293T cell experiments; mean, s.d., and individual datapoints shown for n=3 independent biological replicates. Data in d from HEK 293T cell experiments; mean, s.d., and individual datapoints shown for n=3 independent technical replicates.

FIGS. 32A-D. PRNP clkDNA screens and validation. a, Percentage of sequencing reads harboring indels at the PRNP locus, using CE1.n2 and clkDNAs with varying PBS and PT lengths to install a +6 G-to-T edit. b, Ratio of precise editing to indels at the PRNP locus, using CE1.n2 and clkDNAs with varying PBS and PT lengths. c, Scatter plot depicting percentage of edit and indels at the PRNP locus for CE1.n2 and clkDNAs with varying PS and PT lengths. Highlighted are the clkDNAs that led to some of the highest levels of editing, which we selected for validation. d, Percentage of sequencing reads with precise edits or indels at the PRNP locus with selected clkDNAs which are 2×3′PS protected, using CE1.n2. Data in a-c from HEK 293T cell experiments; mean, s.d., and individual datapoints shown for n=3 independent biological replicates. Data in d from HEK 293T cell experiments; mean, s.d., and individual datapoints shown for n=3 independent technical replicates.

FIGS. 33A-D. GJB2 clkDNA screens and validation. a, Percentage of sequencing reads harboring indels at the GJB2 locus, using CE1.n2 and clkDNAs with varying PBS and PT lengths to install a +2 G deletion. b, Ratio of precise editing to indels at the GJB2 locus, using CE1.n2 and clkDNAs with varying PBS and PT lengths. c, Scatter plot depicting percentage of edit and indels at the GJB2 locus for CE1.n2 and clkDNAs with varying PS and PT lengths. Highlighted are the clkDNAs that led to some of the highest levels of editing, which we selected for validation. d, Percentage of sequencing reads with precise edits or indels at the GJB2 locus with selected clkDNAs which are 2×3′PS protected, using CE1.n2 and two different ngRNAs. Data in a-c from HEK 293T cell experiments; mean, s.d., and individual datapoints shown for n=3 independent biological replicates. Data in d from HEK 293T cell experiments; mean, s.d., and individual datapoints shown for n=3 independent technical replicates.

FIGS. 34A-B. (SEQ ID NO: 595) Effect of PAM disruption on click editing efficiency. Percentage of sequencing reads with precise edits or insertion or deletion mutations (indels) when installing edits that disrupt the PAM of the original target site, using gRNAs targeted to the ACTB or VEGFA loci (a and b, respectively). Data from HEK 293T cell experiments; mean, s.d., and individual datapoints shown for n=3 technical replicates.

FIGS. 35A-I. Comparison of unwanted template-mediated insertions with CEs and PEs. a, Percentage of sequencing reads with precise edits or indels at the ACTB locus with CE1, PE1, or PE3, with a +5 n2b gRNA. Data from HEK 293T cell experiments; mean and s.d. shown for n=3 independent biological replicates. WT, wild-type. b, Schematic of templated polymerization for CEs and PEs. With CEs, the HUHe site on the clkDNA may be blocked by the bound HUHe to prevent read-though, or untethered clkDNAs interacting with the non-target DNA strand without HUHe engagement may lead to template writing. During prime editing experiments, RT-mediated read-through into the sgRNA scaffold sequence can lead to unwanted insertion byproducts (Anzalone et al., Nature, 2019, supra). c,d, (SEQ ID NO(s): 697-703) Distribution and analysis of sgRNA scaffold insertion lengths when using PE3 to install a 3 bp deletion at DNMT1 (+3-5 delAGG), analyzed as described in the Methods section. Example reads are shown in d. e,f, (SEQ ID NO(s): 697-698,704-706) Distribution of template insertion lengths when using CE1 to install a 3 bp deletion at DNMT1 (+3-5 delAGG), analyzed as described in the Methods section. Example reads are shown in f. g, (SEQ ID NO: 41) Schematic of clkDNA configurations encoding poly-T or 2′OMe RNA linkers between the HUHe site and the PT of a clkDNA. h, Editing efficiencies for a 3 bp deletion at DNMT1 (+3-5 delAGG) using the clkDNAs depicted in g. i, Template insertion proportion when using 5×T or 5×mU linker harboring clkDNAs as depicted in g. Data in g and i from HEK 293T cell experiments; mean and s.d. shown for n=3 technical replicates.

FIGS. 36A-D. Cas9-dependent off-target characterization. a, Percentage of sequencing reads harboring precise edits or indels at the ACTB on-target site, with CE1.n2b(+5) or SpCas9 nuclease. b-d, Percentage of sequencing reads harboring indels when using CE1 or SpCas9 nuclease at candidate off-target sites for gRNAs targeting ACTB (CE1.n2b(+5) for +5 G-to-C edit; b), VEGFA (CE1.n1 for a quadruple substitution edit; c), or DNMT1 (CE1.n2(+49) for +3-5 delAGG edit; d). Putative off-target sites were nominated using Cas-OFFinder²⁴. Data in all a-d from HEK 293T cell experiments; mean, s.d., and individual datapoints shown for n=3 independent biological replicates; Control data points were collected from genomic DNA extracted from untransfected cells.

FIGS. 37A-C. PCV2 HUHe off-target characterization. a, Schematic of experimental conditions and workflow to characterize potential PCV2-mediated indels at genomic pseudosites bearing PCV2-HUHe binding motifs, when artificially inducing an R-loop via dCas9 or nCas9 binding. b,c, Percentage of sequencing reads harboring indels in the HUHe R-loop assay, quantified across 16 PCV2 pseudosites, when transfected with the indicated constructs, a gRNA targeting the indicated HUHe pseudosite, and either with a clkDNA (b) or without clkDNA added (c). Data in b and c from HEK 293T cell experiments; mean, s.d., and individual datapoints shown for n=3 independent biological replicates.

FIGS. 38A-G. Characterization of CEs harboring different DDPs and architectures. a,b, Percentage of sequencing reads with precise edits with gRNAs and clkDNAs targeting IL2RB (a) and RNF2 (b) when using CE1.n2 constructs encoding different DNA-dependent polymerases and different construct architectures (fused, unfused or coiled-coil recruited). c-g, Percentage of reads with indels for gRNAs and clkDNAs targeting ACTB, DNMT1 , PRNP, IL2RB and RNF2 (c-g, respectively) when using CE1.n2 constructs encoding different DNA-dependent polymerases and different construct architectures; fused, covalent fusion of eHUH-nCas9-DDP; unfused, separately translated eHUH-nCas9 and DDP proteins; or recruited, where separately translated eHUH-nCas9 and DDP proteins have complementary N5/N6 coiled-coil peptides^{21,22} fused to either protein). EcKlenow, Klenow fragment from E. coli DNA polymerase I (D355A, D357A); Phi29, DNA polymerase from bacteriophage φ29; Phi29 (D169A), 3′-5′ exonuclease-deficient Phi29 DNA polymerase; ePhi29, engineered thermostable Phi29 DNA polymerase (M8R, V51A, M97T, G197D, E221K, Q497P, K512E, F526L); ePhi29 (D169), 3′-5′ exonuclease-deficient ePhi29 (D169A, M8R, V51A, M97T, G197D, E221K, Q497P, K512E, F526L); eB103, engineered thermostable Phi29 ortholog (H73R, A147K, R221Y, A318G, M339L, E359D, K372E, F383L, D384N, A503M, I511V, R544K, T550K. Data in a-g from HEK 293T cell experiments; mean, s.d., and individual datapoints shown for n=3 independent biological replicates.

FIGS. 39A-C. Characterization of PCEs harboring different DDPs and architectures. a, Schematic of the PCE architectures tested. The depicted architectures were tested for their efficiencies to install two edits shown in b and c. b, Editing efficiencies when using various PCE architectures from a to install a C-to-G transversion at the ACTB target site. c, Editing efficiencies when using various PCE architectures from a to install a 3-bp deletion at the DNMT1 target site. Data in b, c from HEK 293T cell experiments; mean, s.d., and individual datapoints shown for n=3 independent biological replicates.

FIGS. 40A-B. Double click editing approach and combining PCEs with recombinases for kilobase DNA insertion. a, Schematic of the PCE architectures and “double-click” approach used in b, which shows editing efficiencies for the depicted architectures with either EcKlenow or ePhi29 DDPs to install 38 bp BxbI attB sequence at the ACTB locus. Data in b from HEK 293T cell experiments; mean, s.d., and individual datapoints shown for n=3 technical replicates.

FIGS. 41A-B. Characterization of PCEs and clkDNA configurations that enable use of endogenous DNA polymerases. a, Editing efficiencies at the ACTB locus (top) or DNMT1 locus (bottom) with a PCE harboring a fused EcKlenow DDP, a PCE containing no DDP (PCV2-nCas9), nCas9 only, or a PCE containing a catalytically inactive EcKlenow DDP. Constructs were tested with clkDNAs containing long, medium, or short PBSs. b, Editing efficiencies at the VEGFA locus with a PCE harboring a fused EcKlenow DDP, a PCE containing no DDP (PCV2-nCas9), nCas9 only, or a PCE containing a catalytically inactive EcKlenow DDP. Constructs were tested with a clkDNA containing a long 17 bp PBS that either contained all DNA bases or 14 3′ 2′OMe bases. Data in a and b from HEK 293T cell experiments; mean, s.d., and individual datapoints shown for n=3 technical replicates.

FIGS. 42A-D. Template jumping click editing. a, Exemplary clkDNA architecture for template jumping click editing. b, Template jumping approach where PBS1 binds the first nick site c, Template jumping approach where PBS1 binds the second nick site. d, Editing efficiencies when using PCE-EcKlenow, PCE-ePhi29, or nCas9 in a template jumping click editing approach to replace a 90 bp genomic segment of DNA at the AAVS1 locus with a 40 bp LoxP site. Data in d from HEK 293T cell experiments; mean, s.d., and individual datapoints shown for n=3 technical replicates.

### DETAILED DESCRIPTION

There has been a recent expansion in the breadth of technologies that seek to generate nucleotide-level changes with higher precision, versatility, and programmability compared to prior approaches²⁵ (while attempting to minimize DNA DSBs and indels), including the development of base editors and prime editors.

Base editors (BEs), which are comprised of fusions of a nickase Cas9 (nCas9; RuvC inactivated) to cytosine or adenine deaminase domains, enable the installation C-to-T^{26,27} or A-to-G^{28,29} nucleotide-level edits (CBEs and ABEs, respectively). BEs are directed by gRNAs to target sites, permitting the deaminase domain to act on DNA in short ~4-8 nucleotide (nt) edit windows on the accessible non-target DNA strand (NTS). BEs achieve higher levels of editing by additionally nicking the target DNA strand (TS), which ensures more stable incorporation of the desired edit on the NTS during DNA repair and/or replication, rather than resolution of the heteroduplex to the cognate sequence.

Prime editors (PEs) are a separate technology that include fusion of nickase Cas9 (HNH inactivated) to a reverse transcriptase (RT), enabling the genetic writing of small edits that are pre-programmed on prime editor guide RNAs (pegRNAs)^{25,30}. PEs can insert, substitute, or delete short sequences by encoding these alterations on a 3′ extension of the pegRNA RNA template (which hybridizes to the NTS to create a transient RNA:DNA duplex), which the RT then utilizes as a primer to polymerize the complementary edit onto the 3′ end of the nicked NTS. The nascent extended NTS sequence generated by the RT creates an extended ‘flap’ that must be preferentially utilized by DNA mismatch repair (MMR) or replication to efficiently install the edit; otherwise, the sequence in the cognate DNA sequence/flap will remain and the edit will not be installed. The generation of a DNA nick on the TS (either distal from the target site or overlapping the edit, the latter of which must be dependent on the edit to avoid indels) can enhance incorporation of the sequence encoded by the nascent flap containing the edit.

Despite the utility of BEs and PEs, both platforms have limitations. Alternate genome editing technologies that involve the use of DNA polymerases or DNA ligases should solve some of these challenges.

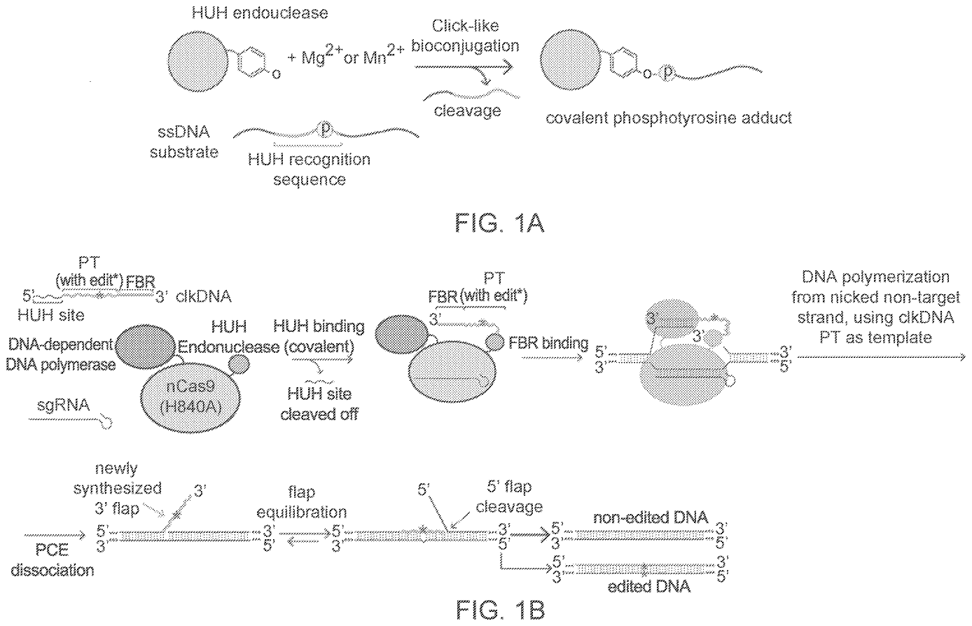

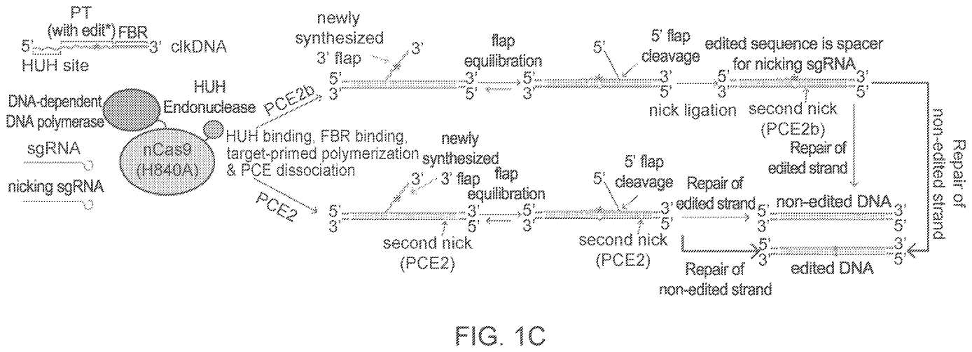

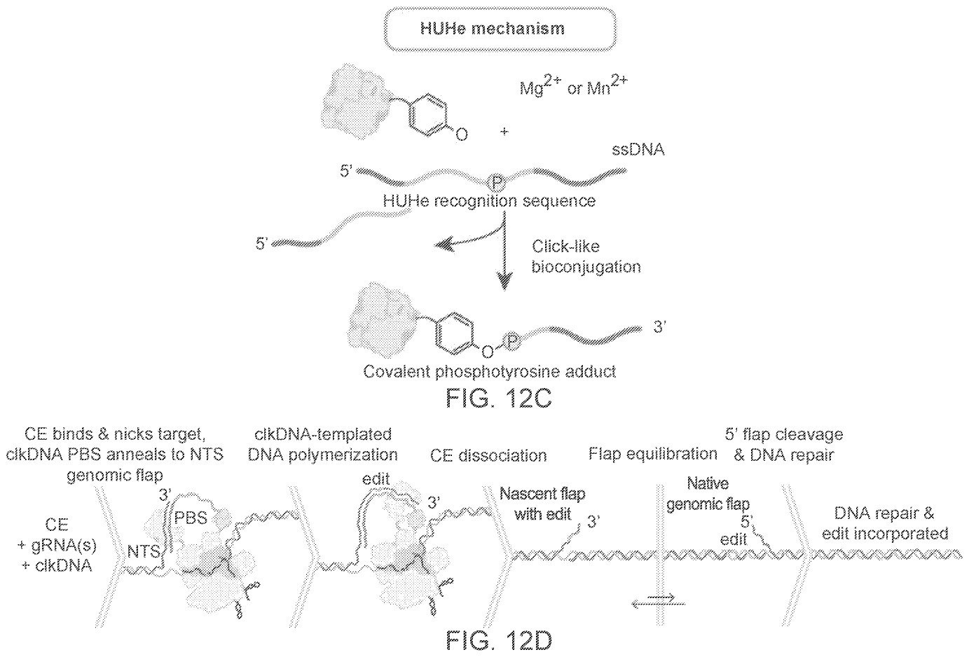

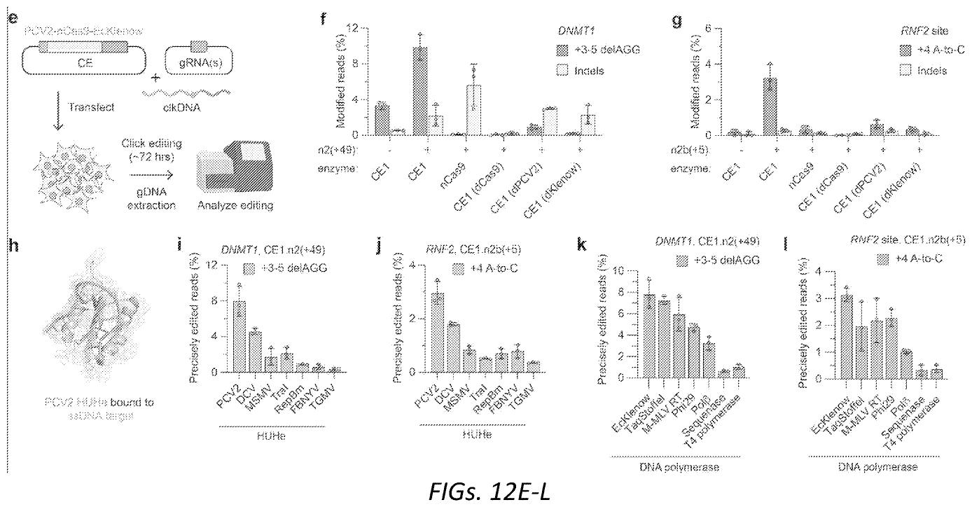

We sought to leverage the potential of DDPs for genome writing, given their ubiquitous presence in cells and potentially advantageous attributes. For example, various classes of DDPs display high-fidelity polymerization, are compatible with inexpensive DNA oligonucleotide (oligo) templates, exhibit high substrate processivity, and are likely to be enzymatically active across nearly any cell type due to high dNTP affinity^{31,32}. The use of DNA oligo templates for genome writing may offer advantages for experiment scalability, template stability, and use in a range of applications, given their ease of synthesis, low cost, high customizability, and that they are a widely used and clinically validated molecule³³. We therefore envisioned that fusion or recruitment of a DDP to nCas9 (FIG. 12a) might create a class of genome writing technologies with distinctions compared to prior approaches. We hypothesized that the use of a ssDNA tethering domain may improve writing efficiency by enabling the localization of the modification-encoding template (FIG. 1a). The tripartite ssDNA could include (1) a recognition sequence for a protein or peptide capable of binding nucleic acids, a polymerization template (PT) containing an edit of interest, and a primer binding site (PBS) that bears homology to the target site's nicked non-target strand (NTS) (FIG. 12a).

An ideal ssDNA recruitment domain would have specificity for the provided ssDNA template, be small in size, have rapid kinetics to catalyze covalent protein-DNA adducts, and not require any specialized and/or expensive modifications. Currently, HUH endonucleases (HUHes) uniquely meet these criteria. HUHes are small proteins spread across all domains of life (FIG. 12b) that carry out diverse ssDNA-specific transactions, including ssDNA viral replication, conjugation, transposition, and others³⁴. Table 6 provides a list of accession numbers for sequences of exemplary HUHes. HUH replication endonucleases and relaxases perform sequence-specific bioconjugation with a ssDNA containing a short recognition sequence (FIG. 12c). Minimized HUH domains have been used in biological applications as “HUH tags”^{35,36}, including as a Cas9-based covalent tether for nuclease-based homology-directed repair (HDR) donor templates³⁷. We envisioned that complexing a DDP and an HUHe with nCas9 could enable HDR- and DSB-independent genome editing. Since HUHes perform “click-like” biochemical reactions for covalent protein-substrate attachment, we named this complex a “click editor” (CE) and the localized ssDNA oligo as a “click DNA” (clkDNA; FIG. 12a). Although clkDNAs are referred to, in some embodiments, the click oligo comprises RNA and thus can also be a click nucleic acid or “clkNA”.

Mechanistically, an SpCas9-H840A nickase-based CE would be programmably directed to a target site by a gRNA to initiate NTS nicking, releasing the endogenous genomic flap³⁸ (FIG. 12d). The CE would covalently tether a clkNA template (encoding a PT which includes the desired edit, and a PBS) to the target site via the HUHe domain. Annealing of the tethered clkNA PBS to the nicked NTS would provide a primer for clkNA-templated DNA polymerization by the CE-fused DDP, resulting in an extended 3′ flap containing the desired edit. Subsequent flap equilibration and DNA repair to incorporate the nascent 3′ flap would lead to precise installation of the edit at the target site (FIG. 12d).

Here we described the localization of DNA polymerases and DNA ligases to nickase Cas9 enzymes (via direct fusion or alternate recruitment methods). These technologies, termed ‘click editors’ (CEs), permit the writing (via polymerase click editors; PCEs) or ligation (via ligase click editors; LCEs) of exogenous DNA sequences onto the accessible 3′ end of the nicked NTS of a Cas9-gRNA target site. Programmable edits are encodable on exogenous DNA templates that are provided in trans (termed click nucleic acids or “clkNAs”) along with the PCE or LCE. The nascent polymerized or ligated nucleic acids on the 3′ end of the nicked NTS create DNA flaps, which must be preferentially incorporated into the locus during DNA repair and replication to avoid reversion to the original unedited sequence. (Primer extension of the genomic flap templated by the clkNA results in an extended genomic 3′ flap that is incorporated into the genome). The edit efficiencies of PCEs and LCEs can be enhanced by localization or recruitment of the clkNA templates compared to simply providing the clkNA in trans. Methods to recruit the clkNA template to the locus-of-interest include the use of HUH endonucleases and/or other nucleic acid tethering approaches. Together, we demonstrate that PCEs and LCEs can install a variety of small nucleotide (nt) level edits and larger sequence insertions and replacements, offering new technological capabilities to edit genomes.

The Click Editors (PCEs and LCEs) comprise a suite of genome editing technologies that leverage the recruitment, localization, or provision in trans of DNA templates to genomic sites for target-specific polymerization or ligation. The use of a DNA nickase to expose 3′ DNA ends (e.g., nSpCas9 with H840A or other analogous mutations, including N863A) provides a substrate for polymerization or ligation, enabling genomic installation of edits independent of DNA DSBs and without a reliance on HDR. We explore various PCE and LCE protein and clkNA nucleic acid architectures and compositions, demonstrating essentiality of clkNA recruitment to achieve more efficient editing. Furthermore, we also show that PCEs can be further employed for targeted, in cellulo diversification when combined with oligo libraries.

Expansion in the methodology of these technologies (e.g., Double Click, Dual-overhang ligation, and other methods as described herein) can also enable more sophisticated edits, such as precise DNA deletion and replacement.

Overall, Click Editing as a genome modification platform holds advantages compared to current technologies in terms of reagent cost and scalability, the labor and expense involved in construct optimization, and portability to alternative RNA-guided enzymes, as well as edit versatility, purity, and potentially efficiency.

### Click Editors—Components and Architectures

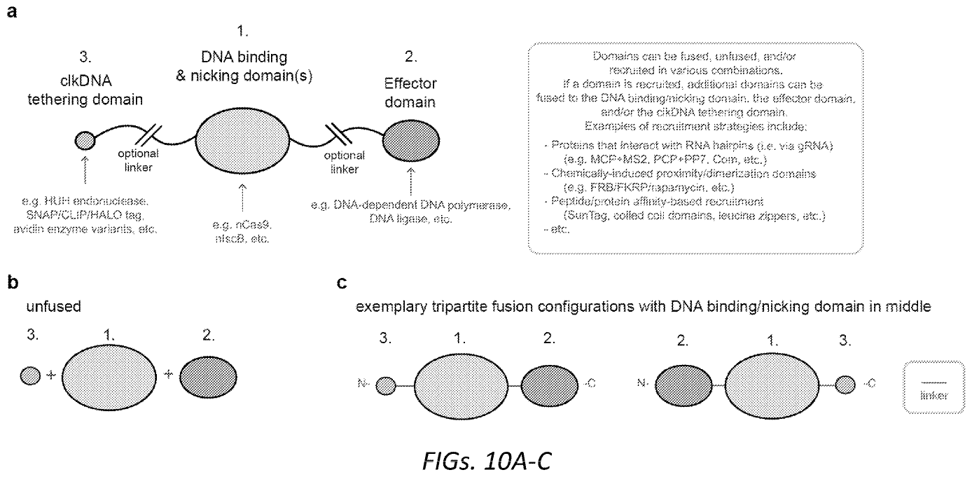

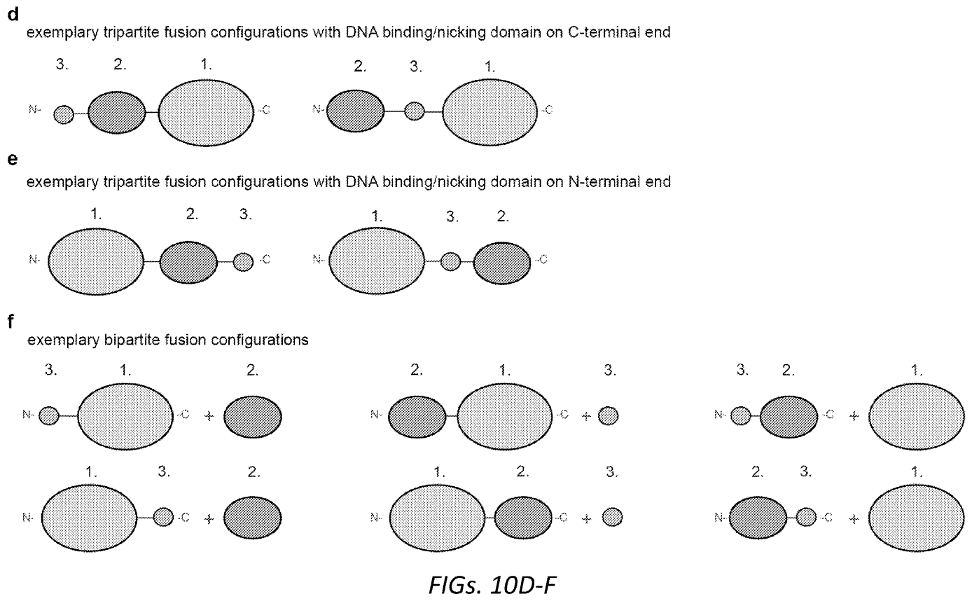

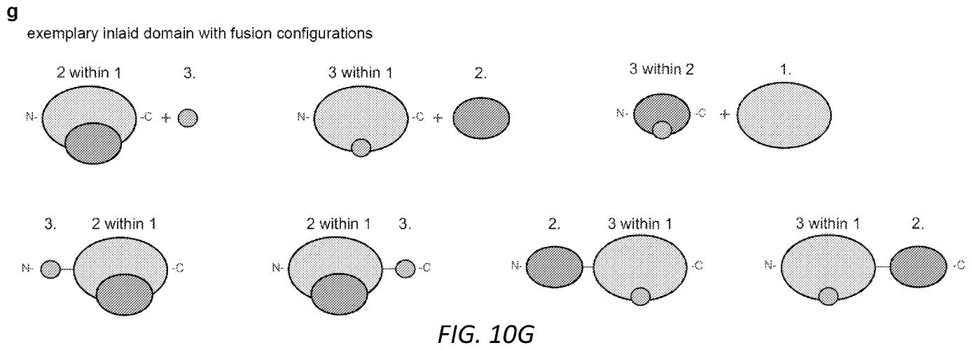

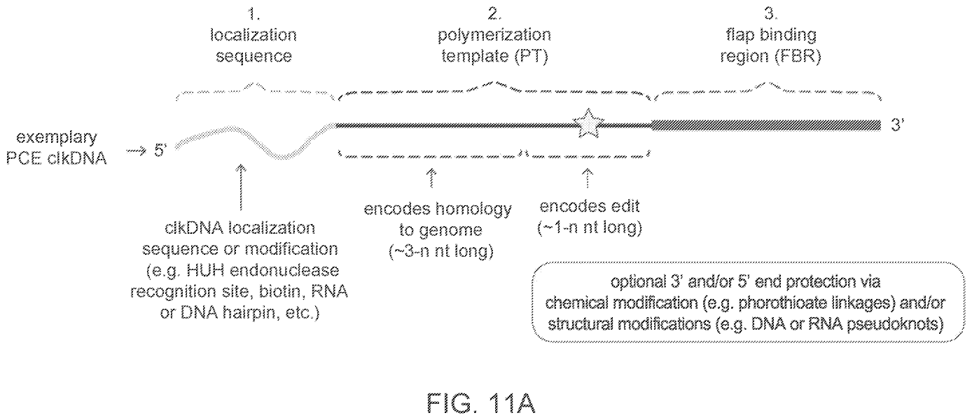

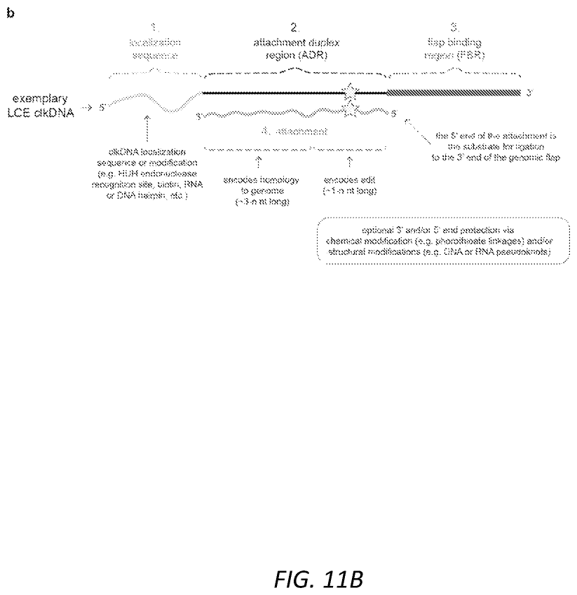

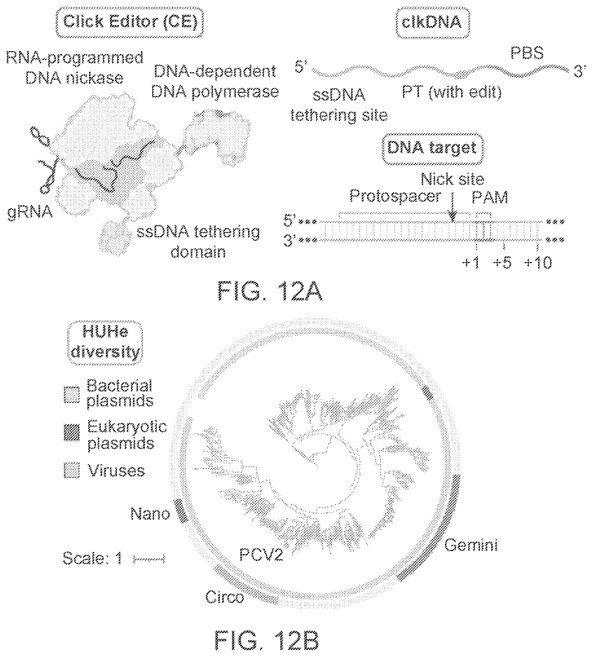

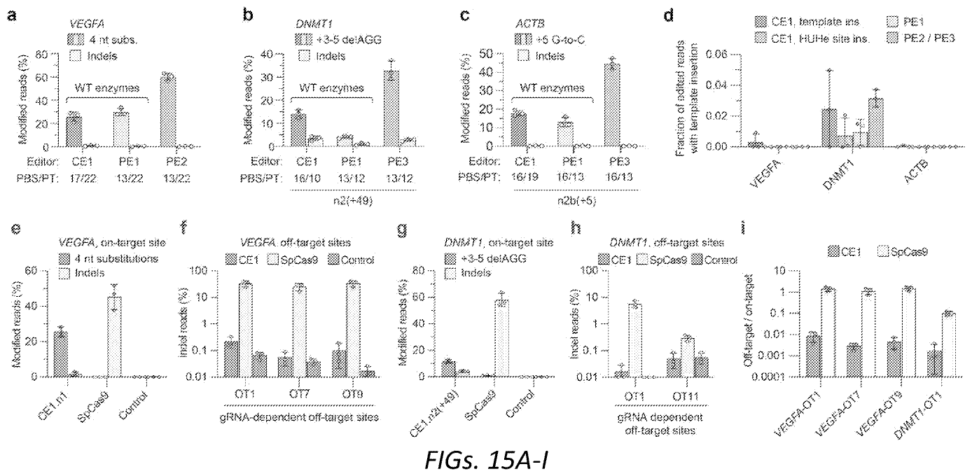

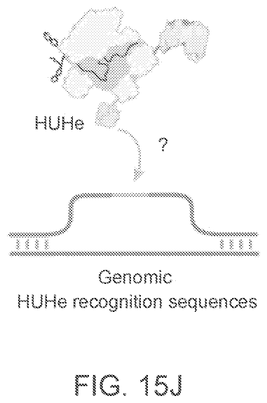

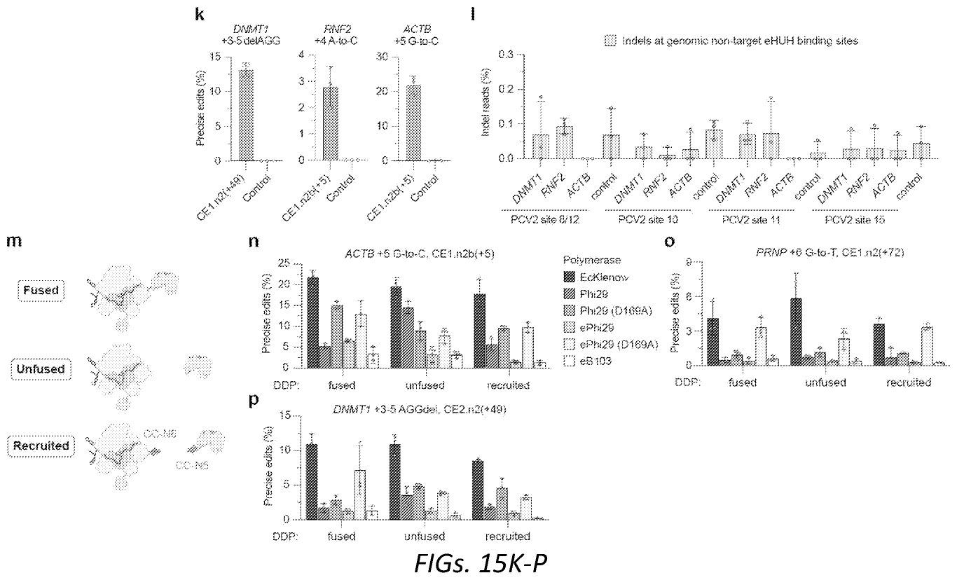

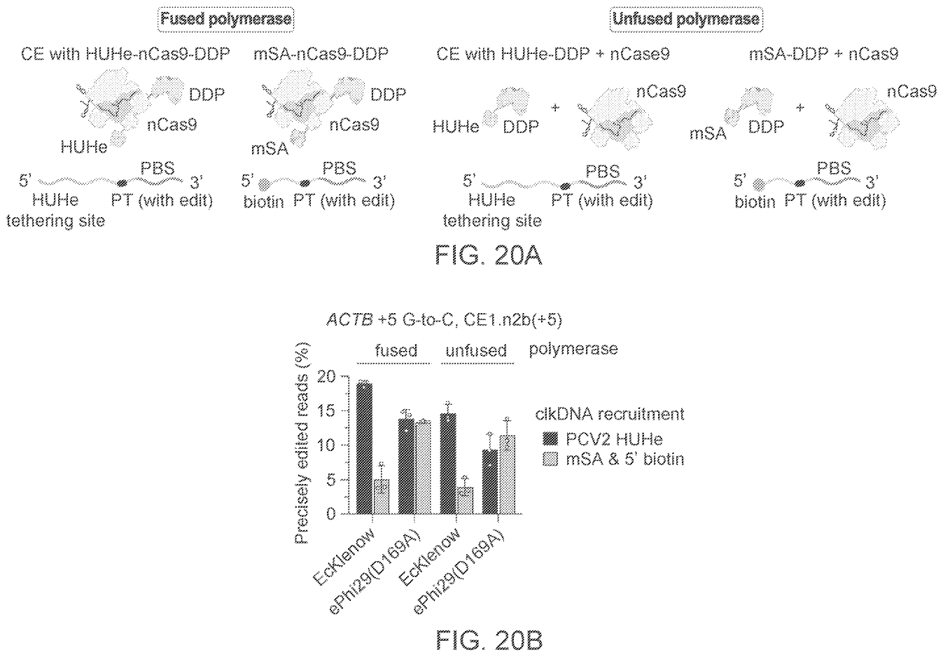

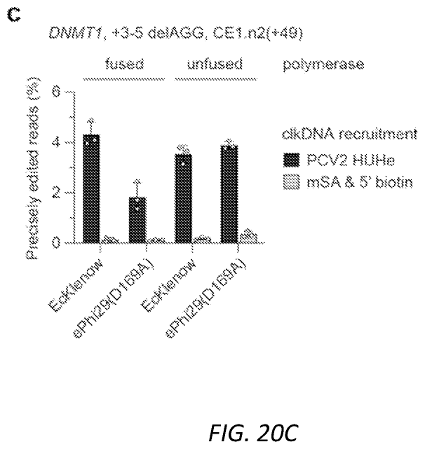

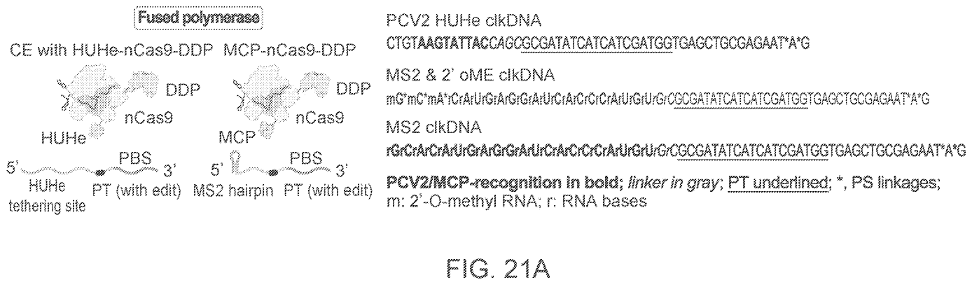

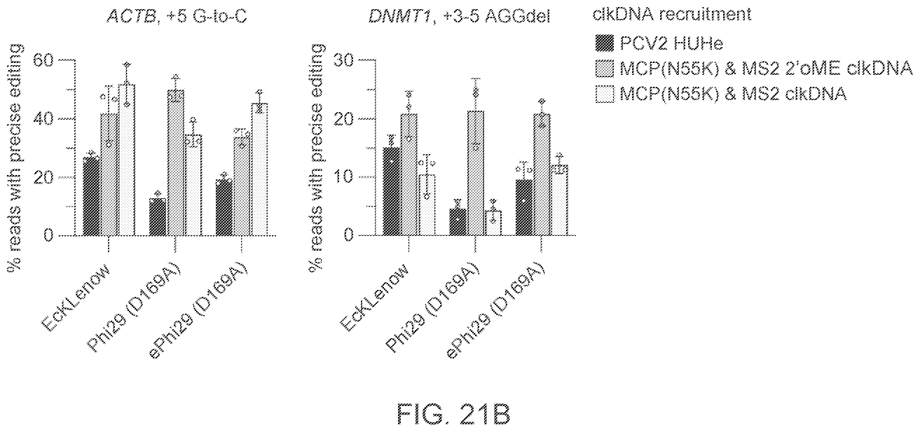

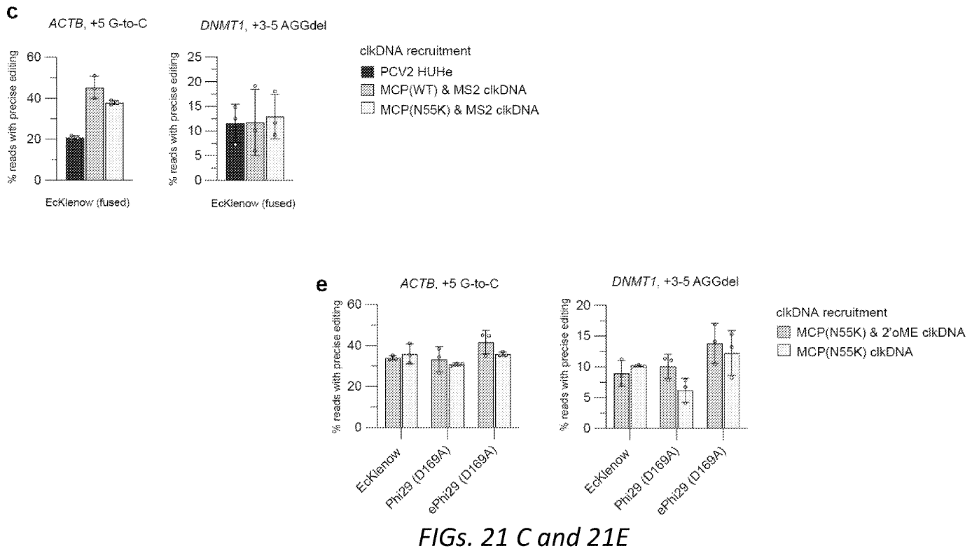

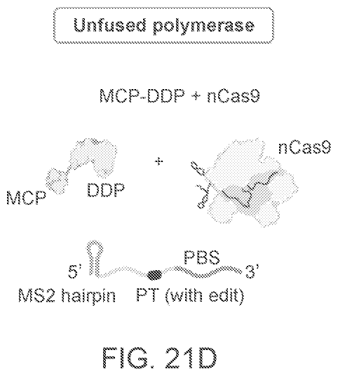

Click editors (PCEs or LCEs) include a DNA binding domain (optionally an RNA-programmable DNA nickase that nicks the non-target strand, or another type of DNA nickase or DNA nuclease), a clkNA tethering domain, and an effector domain (FIG. 10A). The non-target strand (NTS) flap is the substrate for ligation/polymerization with the clkNA. For CRISPR-Cas enzymes, the target strand is paired with the gRNA, so nicking the NTS is expected to lead to more efficient editing. Although the examples provided herein demonstrate fusion proteins with the DNA binding domain in the middle and optional linkers between the DNA binding domain and each of the clkNA tethering domain and the effector domain, Click Editors (CEs) can also be formed from alternative enzymes and architectures (see the illustrations in FIGS. 10A-G). For example, the CEs can be configured in various ways including as unfused components (e.g., as shown in FIG. 10B, FIGS. 15M-P, FIG. 20, FIGS. 21D-E), tripartite fusions with the DNA nickase in the center (e.g., as shown in FIG. 10C), tripartite fusions with the DNA nickase on the C-terminus (e.g., as shown in FIG. 10D), tripartite fusions with the DNA nickase on the N-terminus (e.g., as shown in FIG. 10E), bipartite fusions with separate expression of the third domain (e.g., as shown in FIG. 10F; i.e., the DNA nickase and clkNA tethering domain fused with an unfused effector domain, bipartite fusion of the DNA nickase and effector domain in combination with an unfused clkNA tethering domain, or bipartite fusion of the clkNA tethering domain and the effector domain in combination with an unfused DNA nickase), inlaid compositions (e.g., as shown in FIG. 10G), or other configurations.

Thus, the DNA ligase in LCEs and DNA polymerase in PCEs can be either fused or unfused from the DNA nickase and HUH complex. The HUH endonuclease (or alternate nucleic acid tethering domain) may also be recruited to the DNA binding domain (DBD) via other methods instead of direct fusion (FIGS. 10A-G), including but not limited to recruitment through the gRNA; if any component is unfused, that component can optionally be recruited to the target site through a protein recruitment domain, e.g., phage coat proteins (CP) (coupled with sgRNAs encoding the corresponding RNA recognition hairpin recognized by a given coat protein) (FIG. 21).

Thus, one method to selectively recruit proteins or domains to specific target sites is to fuse the effector protein-of-interest (e.g., ligase or polymerase) to a recruitment domain (e.g., an RNA binding protein, e.g., MCP, PCP, or Com RNA binding protein^{39,40}, e.g., the MS2-coat protein (MCP)) that then interacts with a specific hairpin sequence encoded within that gRNA (e.g., viral RNA sequences MS2, PP7, and com, e.g., an MS2 hairpin) (FIGS. 21A-E). This permits selective recruitment of the effector to the site bound by the primary gRNA; the gRNA targeting the secondary nicking site would not harbor the MS2 hairpin, preventing recruitment to that site.

To recruit proteins to the target site, protein complexes can be formed using a protein recruitment domain coiled-coil (CC) protein domains (FIGS. 15M-P), leucine zippers (LZs), or SunTags, that permit protein:protein interactions (among other types of protein recruitment strategies) can be used. Coiled-coil domains are known in the art, see, e.g., Woolfson, Adv Protein Chem. 2005; 70:79-112 (design of coiled-coil structures and assemblies); Grigoryan and Keating, Curr Opin Struct Biol. 2008 August; 18(4):477-83 (structural specificity in coiled-coil interactions); Reinke et al., Am. Chem. Soc. 2010, 132, 17, 6025-6031 (synthetic coiled-coil interactome, heterospecific modules for molecular engineering); Ljubetic et al., Nature Biotechnology 35:1094-1101 (2017)(coiled-coil protein-origami cages that self-assemble in vitro and in vivo); Fink et al., Nature Chemical Biology 15:115-122 (2019)(orthogonal CC dimerizing domains); Lebar et al., Nature Chemical Biology 16:513-519 (2020) (orthogonal coiled-coil domains); Plaper et al., Scientific Reports 11: 9136 (2021)(coiled-coil heterodimers); and Lainšček et al., Nature Communications 13:3604 (2022)(coiled-coil heterodimer-based recruitment of an exonuclease to CRISPR/Cas). Exemplary coiled-coil sequences include the following:

| | AA Sequence of | SEQ ID |
|---|---|---|
| Name | Exemplary Coiled-Coil Domain | NO(s): |
| P1 | EIQALEE ENAQLEQ ENAALEE EIAQLEY | 1 |
| P2 | KIAQLKE KNAALKE KNQQLKE KIQALKY | 2 |
| P3 | EIQQLEE EIAQLEQ KNAALKE KNQALKY | 3 |
| P4 | KIAQLKQ KIQALKQ ENQQLEE ENAALEY | 4 |
| P3S | EIQQLEE EISQLEQ KNSQLKE KNQOLKY | 5 |
| P4S | KISQLKQ KIQQLKQ ENQQLEE ENSQLEY | 6 |
| P5 | ENAALEE KIAQLKQ KNAALKE EIQALEY | 7 |
| P6 | KNAALKE EIQALEE ENQALEE KIAQLKY | 8 |
| P7 | EIQALEE KNAQLKQ EIAALEE KNQALKY | 9 |
| P8 | KIAQLKE ENQQLEQ KIQALKE ENAALEY | 10 |
| P9 | ENQALEQ KNAQLKQ EIAALEQ EIAQLEY | 11 |
| P10 | KNAQLKE ENAALEE KIQQLKE KIQALKY | 12 |
| P11 | ENQALEQ EIAQLEQ EIAALEQ KNAQLKY | 13 |
| P12 | KNAQLKE KIAALKE KIQQLKE ENQALEY | 14 |
| N5 | EIAALEA KIAALKA KNAALKA EIAALEA | 15 |
| N6 | KIAALKA EIAALEA ENAALEA KIAALKA | 16 |
| AP4 | ELAANEE ELQQNEQ KLAQIKQ KLQAIKY | 17 |

Exemplary combinations of CC domains include P1:P2; P3:P4; P3:P4S; P3S:P4; P3S:P4S; P5:P6; P7:P8; P9:P10; P11:P12; P3:P4; N5:N6; P3:AP4, and P3S:P4S.

Leucine zippers (LZs) are also known in the art. See, e.g., Amoutzias et al., Trends Biochem Sci. 2008 May; 33(5):220-9; Bader and Vogt, (2006). Leucine Zipper Transcription Factors: bZIP Proteins. In: Encyclopedic Reference of Genomics and Proteomics in Molecular Medicine. Springer, Berlin, Heidelberg. Doi. org/10.1007/3-540-29623-9_2180; and Busch and Sassone-Corsi, Trends Genet. 1990 February; 6(2):36-40 (see, e.g., exemplary LZ domain sequences in FIG. 1 of this paper; examples include: GCN4, yAP-1, C/EBP, CREB, CRE-BP1, c-Jun, JunB, JunD, FosB, Fra-1, and c-Fos).

SunTags are described in Tanenbaum et al., Cell. 2014 Oct. 23; 159(3):635-46. Exemplary sequences include: GCN4: LLPKNYHLENEVARLKKLVGER (SEQ ID NO: 18); GCN4 variant: EELLSKNYHLLENEVARLKK (SEQ ID NO: 19); and ScFv-GCN4:

(SEQ ID NO: 20)
GPDIVMTQSPSSLSASVGDRVTITCRSSTGAVTTSNYASWVQEK
PGKLFKGLIGGTNNRAPGVPSRFSGSLIGDKATLTISSLQPEDF
ATYFCALWYSNHWVFGQGTKVELKRGGGGSGGGGSGGGGSSGGG
SEVKLLESGGGLVQPGGSLKLSCAVSGFSLTDYGVNWVRQAPGR
GLEWIGVIWGDGITDYNSALKDRFIISKDNGKNTVYLQMSKVRS
DDTALYYCVTGLFDYWGQGTLVTVSS.

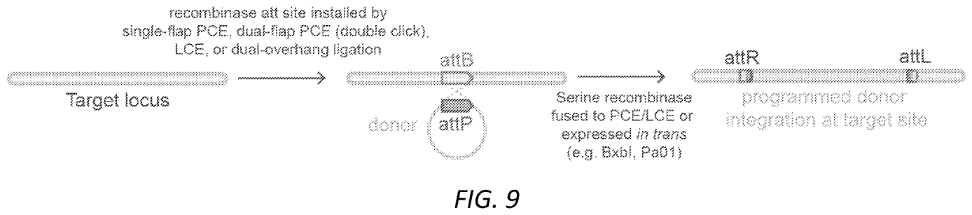

For larger DNA insertion applications, a recombinase can also be fused, unfused, or recruited to the LCE or PCE complex via similar methods described herein (FIG. 9). Here we outline some potential exemplary general configurations of click editor complexes (FIGS. 10A-G), that could encode various nucleic acid recruitment domains (e.g. HUH, though others as described herein can also be substituted for the HUH in the below examples), DNA binding domains (DBDs; e.g. nCas9 or other DNA binding domains, e.g. Cas, IscB, or TnpB nickases or nucleases, etc.), or DNAP polymerases (DNAPs) in fused, unfused, or domain-recruited orientations (e.g. CC domains as an exemplary recruitment strategy):

Exemplary PCE architectures include the following:
All components are fused:
   HUH-DBD-DNAP or DNAP-DBD-HUH
   HUH-DNAP-DBD or DNAP-HUH-DBD
   DBD-HUH-DNAP or DBD-DNAP-HUH
HUH and DBD fused with DNAP separate:
   HUH-DBD with DNAP separate
   DBD-HUH with DNAP separate
HUH, DBD, and CC fused with DNAP fused to CC separate:
   HUH-DBD-CC, CC-HUH-DBD, HUH-CC-DBD, CC-DBD-HUH, DBD-HUH-CC, nCas9-CC-HUH with CC-DNAP or DNAP-CC separate
HUH, DBD, and Suntag fused with DNAP fused to scFV separate:
   HUH-DBD-Suntag, Suntag-HUH-DBD, HUH-Suntag-DBD, Suntag-DBD-HUH, DBD-HUH-Suntag, DBD-Suntag-HUH with scFV-DNAP with scFV-DNAP or DNAP-scFV
HUH and DBD fused with DNAP fused to phage coat proteins (CP) separately:
   HUH-DBD or DBD-HUH with DNAP-CP or CP-DNAP where one or more guide RNA(s) that complexes with the DBD contains an RNA hairpin that binds the CP
   Example CP/hairpin pairs: MCP and MS2, PCP and PP7
HUH is inlaid into the DBD (referred to herein as “[HUH]DBD”) with DNAP separate:
   Any of the above examples with HUH-DBD or DBD-HUH portion replaced by [HUH]DBD
HUH is inlaid into the DBD with DNAP fused:
   [HUH]DBD-DNAP or DNAP-[HUH]DBD
DNAP is inlaid into the DBD with HUH fused:
   HUH-[DNAP]DBD or [DNAP]DBD-HUH
DNAP and HUH are inlaid into the DBD:
   [HUH][DNAP]DBD
HUH and DNAP fused, and DBD separate:
   HUH-DNAP with DBD separate
   DNAP-HUH with DBD separate
Any of the above compositions where a recombinase is additionally fused to the complex containing the DBD, recruited to the DBD, or expressed separately in trans

The DNAP in the above examples for PCEs can be replaced by a DNA ligase for LCEs, or other effectors for alternative click editors. Additionally, for architectures where the DNAP is separate, the DNAP may be one that is endogenous to the host cell whose genome is being edited (where only the tethering domain and the DNA-binding domain complex is provided exogenously into the host cell). The HUH can be replaced by another nucleic acid tethering domain as described herein. Additional compositions are seen in FIGS. 10A-G.

In some embodiments, the click editor is configured such that the clkNA tethering domain (e.g., Phage CP, HUH endonuclease, avidin, SNAP-tag, HALO-tag, CLIP-tag, etc) and effector domain (e.g., polymerase, ligase) are fused together and recruited to the clkNA (which contains the recognition moiety; e.g. MS2, PP7, BoxB, Com, HUH recognition sequence, avidin, SNAP/CLIP/HALO tag substrate), and both are separate from the nCas9, so the Cas9 is separate.

clkNA Tethering Domain

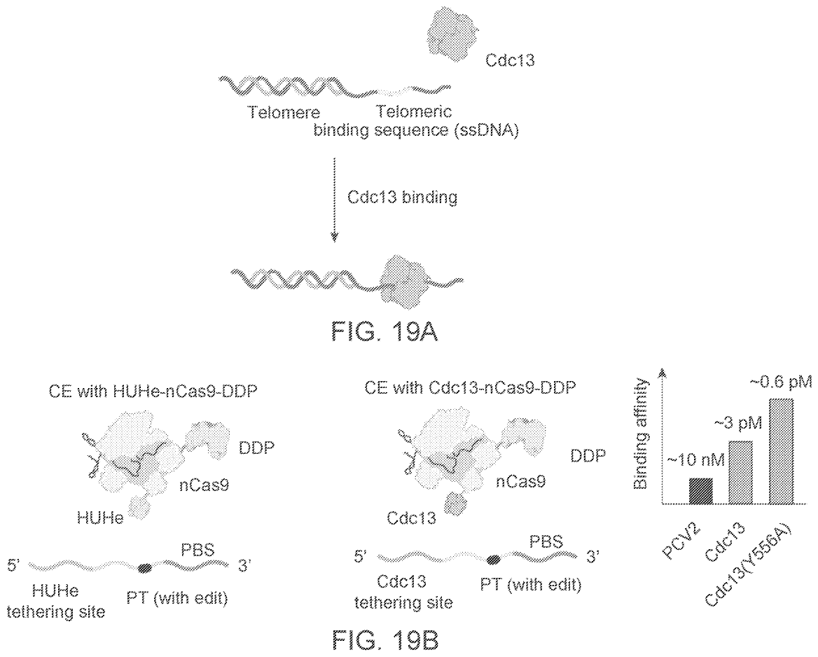

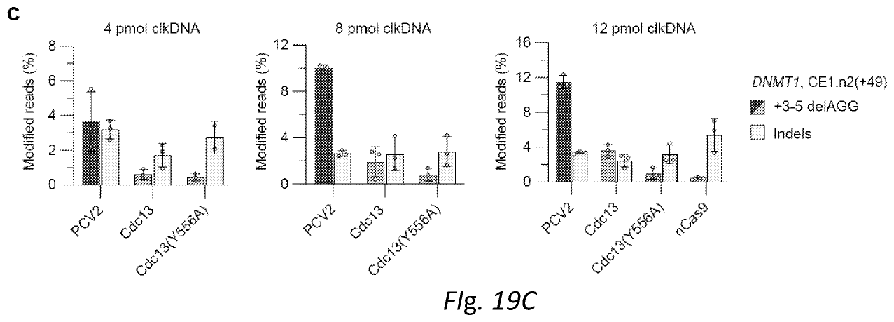

The clkNA tethering domain can include an HUH endonuclease or a TBP, such as Cdc13 (Chandra et al., Genes Dev. 2001 Feb. 15; 15(4):404-14). In some embodiments, HUH endonucleases and TBPs are preferred as they are methods for direct ssDNA-protein binding, thereby also not requiring special and/or expensive chemical modifications for their function. HUH endonucleases can form covalent and direct ssDNA-protein adducts whereas TBPs can non-covalently bind their substrates with picomolar or femtomolar affinity. Exemplary HUH endonucleases include PCV2 HUH domain; DCV HUH domain; FBNYV HUH domain; RepBm HUH domain; TraI relaxase domain; dPCV2 (Y96F) HUH domain, MSMV HUH domain, TGMV HUH domain, ChiSCV-GT306, ChiSCV-GM510, ChiSCV-GM415, and other HUH domains described in Li, L. et al⁴¹ (exemplary sequences in Table A). Exemplary TBPs include Cdc13 and Cdc13(Y555A) (FIG. 19). Alternatively, a different ssDNA/RNA localization moiety can be used, avidin (when the clkDNA is labeled with biotin) (FIG. 20), SNAP-tag (when the clkDNA is labeled with benzylguanine derivatives), CLIP-tag (when the clkDNA is labeled with benzylcytosine derivatives) (or other O⁶-alkylguanine-DNA-alkyltransferase derivatives) and HALO-tag or other haloalkane dehalogenase derivatives (when the clkDNA is labeled with a chloroalkane), or an RNA binding protein, e.g., a phage coat protein (CP), or a phage antitermination signal, such as MCP, MCP(N55K) (FIG. 21), PCP, Com, Phi21 N protein (N^{Phi22})⁸, Phi 22 N protein (N^{Phi22}), lambda N protein (N^{lambda}), an evolved RNA-binding HUH endonuclease⁹, (exemplary sequences in Table B).³⁴

TABLE A
Exemplary HUH endonuclease amino acid sequences

| | | target |
|---|---|---|
| name | aa sequence | sequence* |
| PCV2 | MSPSKKNGRSGPQPHKRWVFTLNNPSEDER | CTGTAAGTATTA |
| HUH | KKIRDLPISLFDYFIVGEEGNEEGRTPHLQGFA | CCAGC (SEQ |
| domain | NFVKKQTFNKVKWYLGARCHIEKAKGTDQQ | ID NO: 41) |
| | NKEYCSKEGNLLMECGAPRSQGQR (SEQ ID | |
| | NO: 21) | |
| DCV | MAKSGNYSYKRWVFTINNPTFEDYVHVLEFC | GTTATATTATTA |
| HUH | TLDNCKFAIVGEEKGANGTPHLQGFLNLRSNA | CCGGC (SEQ |
| domain | RAAALEESLGGRAWLSRARGSDEDNEEYCAK | ID NO: 42) |
| | ESTYLRVGEPVSKGRSSDLAEATSAV (SEQ ID | |
| | NO: 22) | |
| FBNYV | MARQVICWCFTLNNPLSPLSLHDSMKYLVY | GGCTTAGTATTA |
| HUH | QTEQGEAGNIHFQGYIEMKKRTSLAGMKKL | CCCCC (SEQ |
| domain | IPGAHFEKRRGTQGEARAYSMKEDTRLEGP | ID NO: 43) |
| | WEYGEFVP (SEQ ID NO: 23) | |
| RepBm | MSEKKEIVKGRDWTFLVYPESAPENWRTILDE | TGCTTCCGTACT |
| HUH | TFMRWVESPLHDKDVNADGEIKKPHWHILLS | ACGACCCCCCA |
| domain | SDGPITQTAVQKIIGPLNAPNAQKVGSAKGLV | (SEQ ID |
| | RYMVHLDNPEKYQYSLDEIVGHNGADVASY | NO: 44) |
| | FELTA (SEQ ID NO: 24) | |
| TraI | MMSIAQVRSAGSAGNYYTDKDNYYVLGSMG | TTTGCGTGGGGT |
| relaxase | ERWAGRGAEQLGLQGSVDKDVFTRLLEGRLP | GTGGTGCT |
| domain | DGADLSRMQDGSNRHRPGYDLTFSAPKSVSM | (SEQ ID |
| | MAMLGGDKRLIDAHNQAVDFAVRQVEALAST | NO: 45) |
| | RVMTDGQSETVLTGNLVMALFNHDTSRDQEP | |
| | QLHTHAVVANVTQHNGEWKTLSSDKVGKTGF | |
| | IENVYANQIAFGRLYREKLKEQVEALGYETE | |
| | VVGKHGMWEMPGVPVEAFSGRSQTIREAV | |
| | GEDASLKSRDVAALDTRKSKQHVDPEIKMAEW | |
| | MQTLKETGFDIRAYRDAADQRADLRTLTPGPA | |
| | SQDGPDVQQAVTQAIAGLSER (SEQ ID | |
| | NO: 25) | |
| dPCV2 | MSPSKKNGRSGPQPHKRWVFTLNNPSEDERK | CTGTAAGTATTA |
| (Y96F) | KIRDLPISLFDYFIVGEEGNEEGRTPHLQGFA | CCAGC (SEQ |
| HUH | NFVKKQTFNKVKWYLGARCHIEKAKGTDQQNK | ID NO: 41) |
| domain | EFCSKEGNLLMECGAPRSQGQR (SEQ ID | |
| | NO: 26) | |
| RepB | MAKEKARYFTFLLYPESIPSDWELKLETLGVPMA | Tgcttccgtacta |
| HUH | ISPLHDKDKSSIKGQKYKKAHYHVLYIAKNPVTA | cgacccccca |
| domain | DSVRKKIKLLLGEKSLAMVQVVLNVENMYLYLTH | (SEQ ID |
| | ESKDAIAKKKHVYDKADIKLINNFDIDRY (SEQ ID | NO: 44) |
| | NO: 27) | |
| BBTV | MARYVVCWMFTINNPTTLPVMRDEIKYMVYQVER | GGCTTATTATTAC |
| HUH | GQEGTRHVQGYVEMKRRSSLKQMRVFFPGAHLEK | CCCC (SEQ ID |
| domain | RKGSQEEARSYCMKEDTRIEGPFEFGAFKLSCND | NO: 46) |
| | NLFDVIQDMRETHKRPLEYLYDCPNTFDRSKDTL | |
| | YRVQAELNKTRAMNSWRTSFSAWTSEVENIMAQP | |
| | CHRRIIRVYGPNGGEGKTTYAKHLMKTKNAFYSP | |
| | GGKSLDICRLYNYEDIVIFDIPRCKEDYLNYGLL | |
| | EEFKNGIIQSGKYEPVLKVVEYVEVIVMANFLPK | |
| | EGIFSEDRIKLVSC (SEQ ID NO: 28) | |
| WDV | MASSSAPRFRVYSKYLFLTYPQCTLEPQYALDSL | GTTATAATATTAC |
| HUH | RTLLNKYEPLYIAAVRELHEDGSPHLHVLVQNKL | CCCC (SEQ ID |
| domain | RASITNPNALNLRMDTSPFSIFHPNIQAAKDCNQ | NO: 47) |
| | VRDYITKEVDSDANTAEWGTFVAVSTPGRKDRDA | |
| | DMKQIIESSSSREEFLSMVCNRFPFEWSIRLKDF | |
| | EYTARHLFPDPVATYTPEFPTESLICHETIESWK | |
| | NEHLYSESPGRHKSIYICGPTRTGKTSWARSLGT | |
| | HNYYNSLVDFTTYDVNAKYNIIDDIPFKFTPNWK | |
| | CFVGAQRDFTVNPKYGKRKVIRGGIPCIILVNPD | |
| | EDWLKDMTPEQSDYMYSNAVVHYMYEGESFINYS | |
| | FASGEDVTASQ (SEQ ID NO: 29) | |
| TYLCV | MPRLFKIYAKNYFLTYPNCSLSKEEALSQLKNLET | CGTATAATATTAC |
| HUH | PTNKKYIKVCRELHENGEPHLHVLIQFEGKYQCKN | CGGA (SEQ ID |
| domain | QRFFDLVSPNRSAHFHPNIQAAKSSTDVKTYVEKD | NO: 48) |
| | GDFIDFGVFQIDGRSARGGQQSANDAYAEALNSGN | |
| | KSEALNILKEKAPKDYILQFHNLSSNLDRIFSPPL | |
| | EVYVSPFLSSSFNQVPDELEEWVAENVVSSAARPW | |
| | RPNSIVIEGDSRTGKTMWARSLGPHNYLCGHLDLS | |
| | PKVYSNDAWYNVIDDVDPHYLKHFKEFMGAQRDWQ | |
| | SNTKYGKPIQIKGGIPTIFLCNPGPTSSYREYLDE | |
| | EKNISLKNWALKNATFVTLYEPLFASINQGPTQDS | |
| | QEETNKA (SEQ ID NO: 30) | |
| CpCDV | MPSANKNFRFQSKYVFLTYPKCSSQRDDLFEFLWE | AAATTAATATTAC |
| HUH | KLTPFLIFFLGVATELHQDGTTHYHALIQLDKKPW | CGGC (SEQ ID |
| domain | IRDPSFFDFEGNHPNIQPARNSKQVLEYISKDGDI | NO: 49) |
| | KTRGDFRDHKVSPRKSDARWRTIIQTATSKEEYLD | |
| | MIKEEFPHEWATKLQWLEYSANKLFPPQPEAYVSP | |
| | FTESDLRCHEDLAQWRDTHLYQEPRRTGARVPSLY | |
| | ICGPTRTGKTTWARSLGRHNYWNGTIDFTTYDEHA | |
| | TYNVIDDIPFKFVPLWKQLIGCQFDFTVNPKYGKK | |
| | KKIKGGVPSIILTNRDEDWIPAMSEHQKEYFTDNC | |
| | EIHYMDDGETFFARESSSH (SEQ ID NO: 31) | |
| MSMV | MSHTSFRFRAKNVFLTYPRCPIGPEFLCDHLWNLV | ATAATAATATTAC |
| HUH | TPYDPLYVHVAQENHKDGGLHSHVLIQTRIEISTF | GCGC (SEQ ID |
| domain | DPTYFDYTGTSIPGAVVFHPNIQACRNVRDCLAYI | NO: 50) |
| | RKNTINEVSKGAFKCSGAGRPKKQDSAPSRDAKMC | |
| | QIMSSATSRSDYLSMVRGAFPFEWATKLAQFEYSA | |
| | SKLFPDAPTTNAIPSNIDLTCHENIMPWLRDDLYT | |
| | ERGQGRRPRSLYICGRTRTGKTTWARSLGPHNYFQ | |
| | HQVDYFAWSEEATYVVIDDIPFKFCPNWKAIVGSQ | |
| | HDYTVNPKYGKKRKLKGGIPAIILVNEDEDWLMSM | |
| | TTSQIDWFLENCVVYHMTPGESFFNYAE (SEQ ID | |
| | NO: 32) | |
| CLCV | MPRNPKSFRLAARNIFLTYPQCDIPKDEALQMLQT | GCAATAATATTAC |
| HUH | LSWSVVKPTYIRVAREEHSDGSPHLHCLIQLSGKS | CGGA (SEQ ID |
| domain | NIKDARLFDITHPRRSANFHPNIQAAKDTNAVKNY | NO: 51) |
| | ITKDGDYCESGQYKVSGGTKANKDDVYHNAVNAGC | |
| | VEEALAIIKAGDPKTFIVSYHNVRANIERLFTKAP | |
| | EPWAPPFQLSSFTNVPDEMRSWADDYFGRSAAARP | |
| | ERPISIIVEGDSRTGKTMWARALGPHNYLSGHLDF | |
| | NSKVFSNNVEYNVIDDIAPHYLKLKHWKELIGAQR | |
| | DWQSNCKYGRPVQIKGGIPSIVLCNPGEGSSYISF | |
| | LNKEENASLRAWTIKNAKFITLEAPLYQSTAQDC | |
| | (SEQ ID NO: 33) | |
| TGMV | MPSHPKRFQINAKNYFLTYPQCSLSKEESLSQLQA | CGTTTAATATTAC |
| HUH | LNTPINKKFIKICRELHEDGQPHLHVLIQFEGKYC | CGGA (SEQ ID |
| domain | CQNQRFFDLVSPTRSAHFHPNIQRAKSSSDVKTYI | NO: 52) |
| | DKDGDTLVWGEFQVDGRSARGGCQTSNDAAAEALN | |
| | ASSKEEALQIIREKIPEKYLFQFHNLNSNLDRIFD | |
| | KTPEPWLPPFHVSSFTNVPDEMRQWAENYFGKSSA | |
| | ARPERPISIIIEGDSRTGKTMWARSLGPHNYLSGH | |
| | LDLNSRVYSNKVEYNVIDDVTPQYLKLKHWKELIG | |
| | AQRDWQTNCKYGKPVQIKGGIPSIVLCNPGEGASY | |
| | KVFLDKEENTPLKNWTFHNAKFVFLNSPLYQSSTQ | |
| | SS (SEQ ID NO: 34) | |
| TrwC | MLSHMVLTRQDIGRAASYYEDGADDYYAKDGDA | GCGCACCGAAAGG |
| relaxase | SEWQGKGAEELGLSGEVDSKRFRELLAGNIGEG | TGCGTATTGTCTA |
| domain | HRIMRSATRQDSKERIGLDLTFSAPKSVSLQAL | T (SEQ ID |
| | VAGDAEIIKAHDRAVARTLEQAEARAQARQKIQ | NO: 53) |
| | GKTRIETTGNLVIGKFRHETSRERDPQLHTHAV | |
| | ILNMTKRSDGQWRALKNDEIVKRTRYLGAVYNA | |
| | ELAHELQKLGYQLRYGKDGNFDLAHIDRQQIEG | |
| | FSKRTEQIAEWYAARGLDPNSVSLEQKQAAKVL | |
| | SRAKKTSVDREALRAEWQATAKELGIDFSRREWS | |
| | (SEQ ID NO: 35) | |
| NES | MAMYHFQNKFVSKANGQSATAKSAYNSASRIKDF | Acgcgaacggaac |
| relaxase | KENEFKDYSNKQCDYSEILLPNNADDKFKDREYL | gttcgcataagtg |
| domain | WNKVHDVENRKNSQVAREIIIGLPNEFDPNSNIE | cgcccttacggga |
| | LAKEFAESLSNEGMIVDLNIHKINEENPHAHLLC | tttaac (SEQ |
| | TLRGLDKNNEFEPKRKGNDYIRDWNTKEKHNEWR | ID NO: 54) |
| | KRWENVQNKHLEKNGFSVRVSADSYKNQNIDLE | |
| | PTKKEGWKARKFEDETG (SEQ ID NO: 36) | |
| mMobA | MAIYHLTAKTGSRSGGQSARAKADYIQREGKYAR | CCAGTTTCTCGAA |
| relaxase | DMDEVLHAESGHMPEFVERPADYWDAADLYERAN | GAGAAACCGGTAA |
| domain | GRLFKEVEFALPVELTLDQQKALASEFAQHLTGA | GTGCACCCTCCC |
| | ERLPYTLAIHAGGGENPHCHLMISERINDGIERP | (SEQ ID |
| | AAQWFKRYNGKTPEKGGAQKTEALKPKAWLEQTR | NO: 55) |
| | EAWADHANRALERAGH (SEQ ID NO: 37) | |
| ChiSCV- | MHLFTDVVSVDIMTTMQRTHTNAELIRWFKIFRD | AATAGTTACGAAA |
| GM415 | LDIHKWVIGLEEGKGGYGHWQVRCNVRVEVVNDW | A (SEQ ID |
| | TAYLRAVFGWLGPISIWTEECSDKYTYETKEGKY | NO: 56) |
| | WASWDTMGARQQRFGKMRWNQEGAVQALQRTNDR | |
| | EIVVWYDEQGNMGKSWLCGHLFETGQAYYIPPYM | |
| | TSIQSMIQTVASLVLQDRDSGYPPRPLIVIDIPR | |
| | SWKWSTELYTAIEAIKDGLIMDPRYGARPVNIHG | |
| | IKVIVLTNTKPKLDKLSEDRWVLYDPMDYLMML | |
| | (SEQ ID NO: 38) | |
| ChiSCV- | MHLFTEGISVDIMTTMQRTHTNAELIRWFKIFRDL | AATAGTTACGAAA |
| GM510 | DIHKWVIGLEEGKGEYGHWQVRCNVRVEVVNDWTA | A (SEQ ID |
| | YLRAVFGWLGPISIWTEECSDKYTYETKEGKYWAS | NO: 56) |
| | WDTMGARQQRFGKMRWNQEGAVQALQRTNDREIV | |
| | VWYDEGGNMGKSWLCGHLFETGQAYYIPPYMTSI | |
| | QSMIQTVASLVLQDRETGYPPRPLIVIDIPRSWK | |
| | WSTELYTAIEAIKGGLIMDPRYGARPVNIHGTKV | |
| | IVLTNTKPKLDKLSEDRWVLYDPMDYLMML | |
| | (SEQ ID NO: 39) | |
| ChiSCV- | MGARQQRFGKMRWNQEGALQALQRTNDREIVVWY | AATAGTTACGAAA |
| GT306 | DQDGNMGKSWLCGHLYETGQAYYIPPYMTSIQSMIQTVA | A (SEQ ID |
| | SLVLQDRESGYPPRPLIVIDIPRSRKWSTELYTAIEAIK | NO: 56) |
| | DGLIMDPRYGARPVNIHGTKVIVLTNTKPKLDKLSEDRW | |
| | VLYDPLLYMMGH (SEQ ID NO: 40) | |

*nonanucleotide sequence is bolded where known, additional sequence from 5′ and 3′ stem sequence flanking nonanucleotide sequence

TABLE B
Alternative clkNA tethering domains

| name | aa sequence | Target moiety |
|---|---|---|
| SNAP | MDKDCEMKRTTLDSPLGKLELSGCEQGLHRIIFLGKGTSAAD | benzylguanine |
| tag | AVEVPAPAAVLGGPEPLMQATAWLNAYFHQPEAIEEFPVPAL | |
| | HHPVFQQESFTRQVLWKLLKVVKFGEVISYSHLAALAGNPAA | |
| | TAAVKTALSGNPVPILIPCHRVVQGDLDVGGYEGGLAVKEWL | |
| | LAHEGHRLGKPGLG (SEQ ID NO: 57) | |
| CLIP | MDKDCEMKRTTLDSPLGKLELSGCEQGLHRIIFLGKGTSAADA | benzylcytosine |
| tag | VEVPAPAAVLGGPEPLIQATAWLNAYFHQPEAIEEFPVPALHH | |
| | PVFQQESFTRQVLWKLLKVVKFGEVISESHLAALVGNPAATAA | |
| | VNTALDGNPVPILIPCHRVVQGDSDVGPYLGGLAVKEWLLAHE | |
| | GHRLGKPGLG (SEQ ID NO: 58) | |
| HALO | MAEIGTGFPFDPHYVEVLGERMHYVDVGPRDGTPVLFLHGNPT | chloroalkane |
| tag | SSYVWRNIIPHVAPTHRCIAPDLIGMGKSDKPDLGYFFDDHVR | |
| | FMDAFIEALGLEEVVLVIHDWGSALGFHWAKRNPERVKGIAFM | |
| | EFIRPIPTWDEWPEFARETFQAFRTTDVGRKLIIDQNVFIEGT | |
| | LPMGVVRPLTEVEMDHYREPFLNPVDREPLWRFPNELPIAGEP | |
| | ANIVALVEEYMDWLHQSPVPKLLFWGTPGVLIPPAEAARLAKS | |
| | LPNCKAVDIGPGLNLLQEDNPDLIGSEIARWLSTLEISG | |
| | (SEQ ID NO: 59) | |
| mSA-H | MAEAGITGTWYNQHGSTFTVTAGADGNLTGQYENRAQGTGCQN | biotin |
| | SPYTLTGRYNGTKLEWRVEWNNSTENCHSRTEWRGQYQGGAEA | |
| | RINTQWNLTYEGGSGPATEQGQDTFTKVKPSAAS (SEQ | |
| | ID NO: 60) | |
| eMA | MFDASNFKDFSSIASASSSWQNQSGSTMIIQVDSFGNVSGQYV | biotin |
| | NRAQGTGCQNSPYPLTGRVNGTFIAFSVGWNNSTENCNSATGW | |
| | TGYAQVNGNNTEIVTSWNLAYEGGSGPAIEQGQDTFQYVPTTE | |
| | (SEQ ID NO: 61) | |
| MCP | MASNFTQFVLVDNGGTGDVTVAPSNFANGIAEWISSNSRSQAY | MS2 RNA: |
| | KVTCSVRQSSAQNRKYTIKVEVPKGAWRSYLNMELTIPIFATN | ACAUGAGGAUCACC |
| | SDCELIVKAMQGLLKDGNPIPSAIAANSGIY (SEQ ID | CAUGU (SEQ |
| | NO: 62) | ID NO: 69) |
| MCP (N5 | MASNFTQFVLVDNGGTGDVTVAPSNFANGVAEWISSNSRSQAY | MS2 RNA: |
| 5K) | KVTCSVRQSSAQKRKYTIKVEVPKVATQTVGGVELPVAAWRSY | ACAUGAGGAUCACC |
| | LNMELTIPIFATNSDCELIVKAMQGLLKDGNPIPSAIAANSGI | CAUGU (SEQ |
| | Y (SEQ ID NO: 63) | ID NO: 69) |
| PCP | MSKTIVLSVGEATRTLTEIQSTADRQIFEEKVGPLVGRLRLTA | PP7 RNA: |
| | SLRQNGAKTAYRVNLKLDQADVVDCSTSVCGELPKVRYTQVWS | UAAGGAGUUUAUAU |
| | HDVTIVANSTEASRKSLYDLTKSLVATSQVEDLVVNLVPLGR | GGAAACCCUUA |
| | (SEQ ID NO: 64) | (SEQ ID |
| | | NO: 70) |
| Com | MGSMKSIRCKNCNKLLFKADSFDHIEIRCPRCKRHIIMLNACE | com RNA: |
| | HPTEKHCGKREKITHSDETVRYGSTSGHKLNGGGGGMDAKSLT | CUGAAUGCCUGCGA |
| | AWS (SEQ ID NO: 65) | GCAUC (SEQ |
| | | ID NO: 71) |
| Phi21 N | ESKGTAKSRYKARRAELIAERR (SEQ ID NO: 66) | RNA for Phi21 |
| protein | | boxBL: |
| | | ggUUCACCUCUAAC |
| | | CGGGUGAGcc |
| | | (SEQ ID |
| | | NO: 72) |
| P22 N | ATVITYGKSTFAGNAKTRRHERRRKLAIER (SEQ ID | RNA for P22 |
| protein | NO: 67) | boxBL: |
| | | gGCGCUGACAAAGC |
| | | GC (SEQ ID |
| | | NO: 73) |
| lambda | MDAQTRRRERRAEKQAQWKAAN (SEQ ID NO: 68) | RNA for lambda |
| N | | boxBR: |
| protein | | gGCCCUGAAAAAGG |
| | | GC (SEQ ID |
| | | NO: 74) |

mSA-H, (monomeric streptavidin);
eMA, enhanced monomeric avidin

### DNA Binding Domains

The CEs described herein optionally include an RNA-programmable DNA nickase that nicks the NTS, or a nuclease, including nickases from Cas-family enzymes (e.g., Cas9 or Cas12), TnpB-family, or IscB-family enzymes (Table C). See, e.g., Kapitonov et al., J Bacteriol. 2016 Mar. 1; 198(5): 797-807; Karvelis et al., Nature. 2021; 599(7886): 692-696 (TnpB); Koonin and Makarova, PLoS Biol. 2022 January; 20(1): e3001481; Mingarro et al., Gene, 852:147064 (2023); Altae-Tran et al., Science. 2021 October; 374(6563):57-65 (TnpB and IscB); Meers et al., bioRxiv 2023.03.14.532601 (TnpB and IscB); Schuler et al., Science. 2022 Jun. 24; 376(6600):1476-1481; Kato et al., Nat Commun. 2022 Nov. 7; 13(1):6719. Nickases can be generated from wild type RNA-programmable DNA nucleases by the introduction of a mutation of a catalytic RuvC-JJ residue or a mutation of a catalytic HNH residue (Table C). For example, A. warmingii IscB nickases can include an H212A or E157A mutation; IscB nickases from other species can include corresponding mutations; see, e.g., WO 2022/087494. The nickase can also include one or more mutations that increase activity, reduce off-target effects, and/or alter protospacer adjacent motif (PAM) or target adjacent motif (TAM) specificity (Tables D and E). Exemplary Cas9 and Cas2 nickases and mutations are shown in Tables C-E.

TABLE C
List of Exemplary Cas9, Cas12a, and IscB Orthologs
(see WO2018218166 for references)

| | UniProt or GenBank | |
|---|---|---|
| | Accession Number or aa | Nickase Mutations/Catalytic |
| Ortholog | sequence | residues* |
| S. pyogenes Cas9 (SpCas9) | Q99ZW2.1 | D10A (RuvC), E762A (RuvC), |
| | | D839A (HNH), H840A (HNH), |
| | | N854A (HNH partial), N863A |
| | | (HNH), D983A (RuvC), D986A |
| | | (RuvC) |
| S. aureus Cas9 (SaCas9) | J7RUA5.1 | D10A (RuvC), D556A (HNH), |
| | | H557A (HNH), N571A (HNH), |
| | | N580 (HNH) |
| S. thermophilus Cas9 | G3ECR1.2 | D31A (RuvC), D867A (HNH), |
| (St1Cas9) | | H868A (HNH), N882A, (HNH), |
| | | N891A (HNH) |
| N. meningitidis Cas9 | CBA04028.1 | D16A (RuvC), D587A (HNH), |
| (Nme1sCas9) | | H588A (HNH), N602A (HNH), |
| | | N611A (HNH) |
| N. meningitidis DE10444 | WP_002230835.1 | D16A (RuvC), D587A (HNH), |
| Cas9 (Nme2Cas9) | | H588A (HNH), N602A (HNH), |
| | | N611A (HNH) |
| S. pasteurianus Cas9 | BAK30384.1 | D10A (RuvC), D598A (HNH), |
| (SpaCas9) | | H599A (HNH), N613A (HNH), |
| | | N622A (HNH) |
| C. jejuni Cas9 (CjCas9) | Q0P897.1 | D8A (RuvC), D558A (HNH), |
| | | H559A (HNH), N573A (HNH), |
| | | N582A (HNH) |
| F. novicida Cas9 (FnCas9) | A0Q5Y3.1 | D11A (RuvC), D968A (HNH), |
| | | H969A (HNH), N986A (HNH), |
| | | N995A (HNH) |
| P. lavamentivorans Cas9 | A7HP89.1 | D8A (RuvC), D600A (HNH), |
| (PlCas9) | | H601A (HNH), N615A (HNH), |
| | | N624A (HNH) |
| C. lari Cas9 (ClCas9) | G1UFN3.1 | D7A (RuvC), D566A (HNH), |
| | | H567 (HNH) |
| Pasteurella multocida Cas9 | Q9CLT2.1 | D13A (RuvC), D579A (HNH), |
| | | H580A (HNH), |
| F. novicida Cpf1 (FnCpf1) | A0Q7Q2.1 | D917A, E1006A, D1255A (all |
| | | RuvC) |
| M. bovoculi Cpf1 (MbCpf1) | WP_052585281.1 | D986A (RuvC) |
| A. sp. BV3L6 Cpf1 (AsCpf1) | U2UMQ6.1 | D90A8, E993A, Q1226A, |
| | | D1263A (all RuvC) |
| L. bacterium N2006 (LbCpf1) | A0A182DWE3.1 | D832A (RuvC) |
| ogeuIscB | Sequence below | E193A (RuvC) and H247A |
| | | (HNH) |

*for Cas9 and IscB enzymes, the RuvC domain nicks the non-target strand (NTS) DNA and the HNH domain nicks the target strand (TS) DNA. For Cas12a/Cpf1 or TnpB enzymes, the RuvC domain nicks both DNA strands. Mutations abrogate activity.

The sequence of ogeulscB is as follows (from metagenome genome assembly, contig: NODE_25_length_150080_cov_8.882980; contig accession: OGEU01000025.1):

(SEQ ID NO: 75)
MAVVYVISKSGKPLMPTTRCGHVRILLKEGKARVVERKPFTIQL
TYESAEETQPLVLGIDPGRTNIGMSVVTESGESVFNAQIETRNK
DVPKLMKDRKQYRMAHRRLKRRCKRRRRAKAAGTAFEEGEKQRL
LPGCFKPITCKSIRNKEARFNNRKRPVGWLTPTANHLLVTHLNV
VKKVQKILPVAKVVLELNRFSFMAMNNPKVQRWQYQRGPLYGKG
SVEEAVSMQQDGHCLFCKHGIDHYHHVVPRRKNGSETLENRVGL
CEEHHRLVHTDKEWEANLASKKSGMNKKYHALSVLNQIIPYLAD
QLADMFPGNFCVTSGQDTYLFREEHGIPKDHYLDAYCIACSALT
DAKKVSSPKGRPYMVHQFRRHDRQACHKANLNRSYYMGGKLVAT
NRHKAMDQKTDSLEEYRAAHSAADVSKLTVKHPSAQYKDMSRIM
PGSILVSGEGKLFTLSRSEGRNKGQVNYFVSTEGIKYWARKCQY
LRNNGGLQIYV

TABLE D
List of Exemplary High Fidelity and/or PAM-relaxed RGN Orthologs

| Published HF/PAM- | | |
|---|---|---|
| RGN variants | PMID | Mutations* |
| S. pyogenes Cas9 | 26628643 | K810A/K1003A/R1060A eSpCas9(1.0); |
| (SpCas9) eSpCas9 | | K848A/K1003A/R1060A eSpCas9(1.1) |
| S. pyogenes Cas9 | 26735016 | N497A/R661A/Q695A/Q926A (SpCas9-HF1); L169A, |
| (SpCas9) HF1 | | Y450A, |
| S. pyogenes Cas9 | 28931002 | N692A, M694A, Q695A, H698A |
| (SpCas9) HypaCas9 | | |
| S. pyogenes Cas9 | 29431739 | M495V/Y515N/K526E/R661Q; |
| (SpCas9) evoCas9 | | (M495V/Y515N/K526E/R661S; |
| | | M495V/Y515N/K526E/R661L) |
| S. pyogenes Cas9 | 30082871 | R691A |
| (SpCas9) HiFi Cas9 | | |
| S. pyogenes Cas9 | 30082838 | F539S, M763I, K890N |
| (SpCas9) Sniper- | | |
| Cas9 | | |
| S. pyogenes Cas9 | 32144253 | Replacement of L1004-K1014 with two glycines |
| (SpCas9) Blackjack | | |
| S. pyogenes Cas9 | 32187529 | N690C, T769I, G915M, N980K |
| (SpCas9) LZ3 | | |
| S. pyogenes Cas9 | 35236982 | Y1010D, Y1013D, Y1016D, V1018D, R1019D, |
| (SpCas9) SuperFi- | | Q1027D, K1031D |
| Cas9 | | |
| S. pyogenes Cas9 | 36894722 | E1007L |
| (SpCas9) Sniper2L | | |
| S. pyogenes Cas9 | 36894722 | E1007P |
| (SpCas9) Sniper2P | | |
| S. pyogenes Cas9 | 36905119 | K526D |
| (SpCas9) rCas9HF | | |
| S. pyogenes Cas9 | 29512652 | A262T, R324L, S409I, E480K, E543D, M694I, |
| (SpCas9) xCas9 | | E1219V |
| S. pyogenes Cas9 | 30166441 | R1335V, L1111R, D1135V, G1218R, |
| (SpCas9) SpCas9-NG | | E1219F, A1322R, T1337R |
| S. pyogenes Cas9 | 26098369 | D1135V, R1335Q, T1337R (SpCas9-VQR); |
| (SpCas9) | | D1135V/G1218R/R1335E/T1337R (SpCas9-VRER); |
| VQR/VRER | | D1135E |
| S. pyogenes Cas9 | 26098369, | D1135V, G1218R, R1335Q, T1337R |
| (SpCas9) SpCas9- | 26735016 | |
| VRQR | | |
| S. aureus Cas9 | 26524662 | E782K/N968K/R1015H |
| (SaCas9)-KKH | | |
| enAsCas12a | U.S. Ser. No. 15/960,271 | One or more of: E174R, S170R, S542R, K548R, |
| | | K548V, N551R, N552R, K607R, K607H, e.g., |
| | | E174R/S542R/K548R, E174R/S542R/K607R, |
| | | E174R/S542R/K548V/N552R, S170R/S542R/K548R, |
| | | S170R/E174R, E174R/S542R, S170R/S542R, |
| | | E174R/S542R/K548R/N551R, E174R/S542R/K607H, |
| | | S170R/S542R/K607R, or |
| | | S170R/S542R/K548V/N552R |
| enAsCas12a-HF | U.S. Ser. No. 15/960,271 | One or more of: E174R, S542R, K548R, e.g., |
| | | E174R/S542R/K548R, E174R/S542R/K607R, |
| | | E174R/S542R/K548V/N552R, S170R/S542R/K548R, |
| | | S170R/E174R, E174R/S542R, S170R/S542R, |
| | | E174R/S542R/K548R/N551R, E174R/S542R/K607H, |
| | | S170R/S542R/K607R, or |
| | | S170R/S542R/K548V/N552R, with the addition of one |
| | | or more of: N282A, T315A, N515A and K949A |
| enLbCas12a(HF) | U.S. Ser. No. 15/960,271 | One or more of T152R, T152K, D156R, D156K, |
| | | Q529K, G532R, G532K, G532Q, K538R, K538V, |
| | | D541R, Y542R, M592A, K595R, K595H, K595S or |
| | | K595Q, e.g., D156R/G532R/K538R, |
| | | D156R/G532R/K595R, D156R/G532R/K538V/Y542R, |
| | | T152R/G532R/K538R, T152R/D156R, D156R/G532R, |
| | | T152R/G532R, D156R/G532R/K538R/D541R, |
| | | D156R/G532R/K595H, T152R/G532R/K595R, |
| | | T152R/G532R/K538V/Y542R, optionally with the |
| | | addition of one or more of: N260A, N256A, K514A, |
| | | D505A, K881A, S286A, K272A, K897A |
| enFnCas12a(HF) | U.S. Ser. No. 15/960,271 | One or more of T177A, K180R, K180K, E184R, |
| | | E184K, T604K, N607R, N607K, N607Q, K613R, |
| | | K613V, D616R, N617R, M668A, K671R, K671H, |
| | | K671S, or K671Q, e.g., E184R/N607R/K613R, |
| | | E184R/N607R/K671R, E184R/N607R/K613V/N617R, |
| | | K180R/N607R/K613R, K180R/E184R, E184R/N607R, |
| | | K180R/N607R, E184R/N607R/K613R/D616R, |
| | | E184R/N607R/K671H, K180R/N607R/K671R, |
| | | K180R/N607R/K613V/N617R, optionally with the |
| | | addition of one or more of: N305A, N301A, K589A, |
| | | N580A, K962A, S334A, K320A, K978A |
| chimeric Cas9 | 30718489 | S. aureus Cas9 with PAM interaction domain from |
| cCas9 | | SaCas9 orthologues, expands recognition and |
| | | targetability of NNVRRN, NNVACT, NNVATG, |
| | | NNVATT, NNVGCT, NNVGTG, and NNVGTT PAM |
| | | sequences |
| Streptococcus | doi.org/10.1 | Recognizes 5′-NAA-3′ PAM |
| macacae (Smac) | 101/429654 | |
| Cas9 NCTC 11558 | | |
| Spy-mac Cas9, | 32424114 | Recognizes 5′-NAA-3′ PAM |
| Smac-py Cas9, | | |
| iSpyMac | | |
| N. meningitidis | 30581144 | Recognizes N₄CC PAM |
| Nme2Cas9 | | |
| SpG Cas9 (SEQ-ID | 32217751; | SpCas9 variant capable of targeting NGN PAMs |
| 158) | WO | D1135L/S1136W/G1218K/E1219Q/R1335Q/T1337R |
| | 2021151085 | Also as SpG-HF1 in combination with |
| | | N497A/R661A/Q695A/Q926A |
| SpRY Cas9 (SEQ-ID | 32217751; | SpCas9 variant capable of targeting NRN > NYN PAMs |
| 157) | WO | SpRY(A61R/L1111R/D1135L/S1136W/G1218K/E1219Q/ |
| | 2021151085 | N1317R/A1322R/R1333P/R1335Q/T1337R); also |
| | | as SpRY-HF1 in combination with |
| | | N497A/R661A/Q695A/Q926A |
| S. pyogenes Cas9 | 32042170 | D10T(optional)/1322V/S409I/E427G/R654L/R753G/ |
| (SpCas9) SpCas9- | | R1114G/D1135N/E1219V/D1332N/R1335Q/T1337N/ |
| NRCH | | S1338T/H1349R |
| S. pyogenes Cas9 | 32042170 | D10T(optional)/1322V/S409I/E427G/R654L/R753G/ |
| (SpCas9) SpCas9- | | R1114G/D1135N/V1139A/D1180G/E1219V/Q1221H/ |
| NRRH | | A1320V/R1333K |
| S. pyogenes Cas9 | 32042170 | D10T(optional)/1322V/S409I/E427G/R654L/R753G/ |
| (SpCas9) SpCas9- | | R1114G/D1135N/D1180G/G1218S/E1219V/Q1221H/ |
| NRTH | | P1249S/E1253K/P1321S/D1332G/R1335L |

*predicted based on UniRule annotation on the UniProt database.

TABLE E
List of Exemplary SpCas9 Activity-Altering Mutations

| | PMID or | |
|---|---|---|
| Name of enzyme | patent ID | Mutations |
| S. pyogenes Cas9 (SpCas9) | 26098369, | L1111H, G1218R, T1337R |
| SpCas9-VRQR | 26735016 | |
| S. pyogenes Cas9 (SpCas9) | PCT/US2021/ | S55R, A61R, R221K, G366R, N394K, |
| (various mutations) | 014933 | G1104K, G1104R, L1111R, T1138K, |
| | | T1138R, V1139T, V1139S, K1151R, |
| | | K1153R, K1200R, G1218R, E1243K, |
| | | L1245V, E1253K, A1285R, A1285K, |
| | | K1289R, T1314R, N1317R, G1219R, |
| | | G1319H, A1320S, A1320T, A1322S, |
| | | A1322T, A1322R, D1332R, D1332K, |
| | | D1332H, D1332Q, D1332N, |
| | | A61R/N1317R, G1104K/N1317R, |
| | | A61R/G1104K, S55R/G1104K, |
| | | S55R/A61R, S55R/N1317R, |
| | | G1104K/T1314R, A61R/T1314R, |
| | | T1314R/N1317R, S55R/T1314R, |
| | | A61R/G1104R, G1104R/N1317R, |
| | | S55R/G1104R, G1104R/T1314R, |
| | | G366R/G1104K, S55R/G366R, |
| | | A61R/G366R, G366R/N1317R, |
| | | G366R/T1314R, G366R/G1104R, |
| | | G1104K/A1285R, A61R/A1285R, |
| | | A1285R/N1317R, S55R/A1285R, |
| | | A1285R/T1314R, G1104R/A1285R, |
| | | G366R/A1285R, G1104K/A1285K, |
| | | A61R/A1285K, A1285K/N1317R, |
| | | S55R/A1285K, A1285K/T1314R, |
| | | G1104R/A1285K, G366R/A1285K, |
| | | G1104K/D1332R, A61R/D1332R, |
| | | D1332R/N1317R, S55R/D1332R, |
| | | D1332R/T1314R, G1104R/D1332R, |
| | | G366R/D1332R, A1285K/D1332R, |
| | | A1285R/D1332R, |
| | | A61R/G1104K/N1317R, A61R/L1111R, |
| | | A61R/A1322R, A61R/L1111R/A1322R, |
| | | G1104K/L1111R, G1104K/A1322R, |
| | | G1104K/L1111R/A1322R, |
| | | N1317R/L1111R, N1317R/A1322R, |
| | | N1317R/L1111R/A1322R, |
| | | A61R/N1317R/L1111R, |
| | | A61R/N1317R/A1322R, |
| | | A61R/N1317R/L1111R/A1322R, |
| | | G1104K/N1317R/L1111R, |
| | | G1104K/N1317R/A1322R, |
| | | G1104K/N1317R/L1111R/A1322R, |
| | | A61R/G1104K/L1111R, |
| | | A61R/G1104K/A1322R, |
| | | A61R/G1104K/L1111R/A1322R, |
| | | S55R/L1111R, S55R/A1322R, |
| | | S55R/L1111R/A1322R, G366R/L1111R, |
| | | G366R/A1322R, G366R/L1111R/ |
| | | A1322R, N394K/L1111R, |
| | | N394K/A1322R, |
| | | N394K/L1111R/A1322R, |
| | | A1285K/L1111R, A1285K/A1322R, |
| | | A1285K/L1111R/A1322R, |
| | | D1332K/L1111R, D1332K/A1322R, |
| | | D1332K/L1111R/A1322R, R691A, |
| | | M495V, Y515N, K526E, R661Q, |
| | | R661L, R661S, Y450A/Q695A, |
| | | L169A/Q695A, Q695A/Q926A, |
| | | Q695A/D1135E, Q926A/D1135E, |
| | | Y450A/D1135E, L169A/Y450A/Q695A, |
| | | L169A/Q695A/Q926A, |
| | | Y450A/Q695A/Q926A, |
| | | R661A/Q695A/Q926A, |
| | | N497A/Q695A/Q926A, |
| | | Y450A/Q695A/D1135E, |
| | | Y450A/Q926A/D1135E, |
| | | Q695A/Q926A/D1135E, |
| | | L169A/Y450A/Q695A/Q926A, |
| | | L169A/R661A/Q695A/Q926A, |
| | | Y450A/R661A/Q695A/Q926A, |
| | | N497A/Q695 A/Q926A/D1135E, |
| | | R661A/Q695A/Q926A/D1135E, and |
| | | Y450A/Q695A/Q926A/D1135E; |
| | | N692A/M694A/Q695A/H698A, |
| | | N692A/M694A/Q695A/H698A/Q926A; |
| | | N692A/M694A/Q695A/Q926A; |
| | | N692A/M694A/H698A/Q926A; |
| | | N692A/Q695A/H698A/Q926A; |
| | | M694A/Q695A/H698A/Q926A; |
| | | N692A/Q695A/H698A; |
| | | N692A/M694A/Q695A; |
| | | N692A/H698A/Q926A; |
| | | N692A/M694A/Q926A; |
| | | N692A/M694A/H698A; |
| | | M694A/Q695A/H698A; |
| | | M694A/Q695A/Q926A; |
| | | Q695A/H698A/Q926A; |
| | | G582A/V583A/E584A/D585A/N588A/ |
| | | Q926A; |
| | | G582A/V583A/E584A/D585A/N588A; |
| | | T657A/G658A/W659A/R661A/Q926A; |
| | | T657A/G658A/W659A/R661A; |
| | | F491A/M495A/T496A/N497A/Q926A; |
| | | F491A/M495A/T496A/N497A; |
| | | K918A/V922A/R925A/Q926A; or |
| | | 918A/V922A/R925A; K855A; |
| | | K810A/K1003A/R1060A; |
| | | K848A/K1003A/R1060A; |
| | | M495V/Y515N/K526E/R661Q; |
| | | M495V/Y515N/K526E/R661L; or |
| | | M495V/Y515N/K526E/R661S |
| S. pyogenes Cas9 (SpCas9) | 32217751 | A61R, G1104K (not present in final |
| SpRY | | SpRY enzyme), L1111R, N1317R, |
| | | A1322R |
| S. pyogenes Cas9 (SpCas9) | 30166441 | L1111R, A1322R |
| SpCas9-NG | | |
| S. pyogenes Cas9 (SpCas9) | 29203891 | R221K, L244Q, N349K, E1243K, |
| (various mutations) | | L1245V, E1253K |
| S. pyogenes Cas9 (SpCas9) | 26990992 | N1286Q, I1331F, D1332K, |
| SpCas9-QQR | | |
| S. pyogenes Cas9 (SpCas9) | 25207793 | D147Y, P411T |
| iCas9 | | |
| S. pyogenes Cas9 (SpCas9) | 36917274 | L1206P |
| SpCas9-L1206P | | |

### Effector Proteins

The CEs described herein further include effector proteins that have DNA polymerase or ligase activity, e.g., DNA-dependent DNA polymerases of family A, B, C, D, X, or Y, or reverse transcriptases (for polymerase click editors (PCEs)), or DNA ligase (for ligase click editors (LCEs)). Exemplary polymerases include E. coli Klenow (EcKlenow, optionally with the D355A and/or E357A mutations that deactivate its 3′-5′ exonuclease domain); Taq Stoffel; Pol-Beta; Pol-Beta+Sso7d; Phi29 DNA Polymerase (D169A); Sequenase; T4 DNA Polymerase, E. coli dKlenow (optionally with the D355A, E357A, D705A, and/or D882A mutations), etc. Exemplary reverse transcriptases are described in more detail below. Exemplary DNA Ligases include T3; T4; T7; ChlV (SplintR); PhiKMV; Vaccinia; and dT4.

Previous literature on Taq DNA polymerase demonstrated activity-enhancing mutations at amino acid positions 732 (D732N)⁴², 507⁴³, 543⁴⁴, 605/617⁴⁵, 685/686/687 (U.S. Ser. No. 11/046,939B2), and 742/743 with or without a basic residue insertion of length 3 or length 9 between positions 738 and 739⁴⁶. EcKlenow is also structurally homologous to TaqStoffel, hinting that analogous mutations may also increase the activity of EcKlenow. Moreover, non-specific DNA binding domains (e.g. villin headpiece, supercharged villin headpiece, Sso7d, NeqSSB, etc.) fused to the N- or C-terminus of DNA polymerases have been shown to increase the DNA affinity, stability, and processivity of the polymerase⁴⁷⁻⁴⁹. Exchange of the 3′-5′ exonuclease domain of TaqStoffel with that of EcKlenow may also endow TaqStoffel with proofreading capability, as has been done previously⁵⁰. Thus, the present compositions and methods can use a DNA polymerase, such as EcKlenow or TaqStoffel, which include one or more of these modifications.

### Reverse Transcriptases (RTs), Reduced Size RTs, and Variant RTs

The present compositions and methods can use any RT, including Group II introns. Group II introns are retroelements that consist of a self-splicing ribozyme and an intron encoded protein (IEP) which functions as a reverse transcriptase (RT), DNA endonuclease, and RNA maturase.

In some embodiments, the pentamutant Moloney Murine Leukemia Virus reverse transcriptase (MMLV-RT) can be used. The group II intron RT (commercially available as “MarathonRT”) from Eubacterium rectale (E.r.) has been shown to display superior intrinsic RT processivity compared to Superscript IV. As shown herein, substitution of the M-MLV RT in a PE with MarathonRT or other RTs resulted in efficient prime editing in the HEK293T cell line. Thus the RT can be, e.g., MarathonRT, GsI-IIC RT, and MMLV-RT variants, e.g., PE2 MMLV RT (with D200N, T306K, W313F, T330P, L603W mutations), or MMLV or PE2 MMLV RT truncations (e.g., truncations 2, 5, or 6), MMLV variants encoded in PE6a-PE6g¹⁰ (e.g. or M-MLV RT (T128N, V223Y, D200C, with or without RNaseH domain truncation of M-MLV), Gs RT or Gs RT (A16E, L37P, A123V), Tfl RT or Tfl RT (P70T, G72V, S87G, M102I, K106R, K118R, I128V, L158Q, F269L, A363V, K413E, S492N) or Tfl RT (P70T, G72V, S87G, M102I, K106R, K118R, 1128V, L158Q, F269L, A363V, K413E, S492N, K118R, S188K, 1260L, S297Q, R288Q), Ec48 RT or Ec48 RT (E60K, K87E, E165D, D243N, R267I, E279K, K318E, K343N), KORV RT, WMSV RT, AVIRE RT, BAEMV RT, PERV RT, MMTV RT, Ty3 RT, GALV RT, SRV2 RT, MPMV RT, Vp96 RT, ASLV RT), RT HFV, HERV, LtrA, HERV-Kcon, Tel4c, GsI-IIC, Ma-Int5, or engineered Marathon (optionally with D14R, N26R, D74R, N116K, or N197R mutations)

Additional exemplary alternative RTs include those listed in Table F, below.

TABLE F
Alternative reverse transcriptases

| | NCBI or Uniprot | Reverse Transcriptase |
|---|---|---|
| Organism | Acc. No. or Source | Type |
| Geobacillus | E2GM63 (uniport) | Group II Intron |
| stearothermophilus* | | |
| Lactococcus lactis | AAB06503.1 | Group II Intron |
| subsp. lactis | | |
| Thermosynechococcus | BAC08171.1 | Group II Intron |
| elongatus BP-1 | | |
| Sinorhizobium meliloti | WP_010967953.1 | Group II Intron |
| Methanosarcina | AAM07961.1 | Group II Intron |
| acetivorans C2A | | |
| Enterobacter cloacae | AEC33268.1 | Group II Intron |
| Clostridium | NP_350100.1 | Group II Intron |
| acetobutylicum ATCC | | |
| 824 | | |
| Bacillus halodurans | BAA90841.1 | Group II Intron |
| Pseudomonas | AAB68949.1 | Group II Intron |
| alcaligenes | | |
| Pseudomonas putida | CAB81565.1 | Group II Intron |
| Streptococcus | CAC35989.1 | Group II Intron |
| agalactiae | | |
| Roseburia intestinalis | D4L313 (uniprot) | Group II Intron |
| Eubacterium rectale | CBK92290.1 | Group II Intron |
| (marathonRT) | | |
| Streptococcus | WP_013851921.1 | Group II Intron |
| pasteurianus | | |
| Shigella sonnei | WP_077124660.1 | Group II Intron |
| Saccharomyces | NP_009310.1 | Group II Intron (yeast) |
| cerevisiae S288C | | |
| (yeast) | | |
| Saccharomyces | NP_009309.1 | Group II Intron (yeast) |
| cerevisiae S288C | | |
| (yeast) | | |
| Bordetella virus BPP1 | AAR97672.1 | Diversity Generating |
| | | Retroelement |
| ANMV-1 virus | AJP62064.1 | Diversity Generating |
| | | Retroelement |
| Bacteroides phage p00 | DAC76693.1 | Diversity Generating |
| | | Retroelement |
| Treponema denticola | AAS12785.1 | Diversity Generating |
| ATCC 35405 | | Retroelement |
| archaeon | AJF63168.1 | Diversity Generating |
| GW2011_AR20 | | Retroelement |
| Baboon endogenous | YP_009109694.1 | Retrovirus |
| virus strain M7 | | |
| Feline leukemia virus | NP_047255.1 | Retrovirus |
| Human foamy virus | CAA68999.1 | Retrovirus |
| Feline | AAB59937.1 | Retrovirus |
| immunodeficiency | | |
| virus | | |
| Human Endogenous | Nam Lee, et. al | Retrovirus |
| Retrovirus K | (2007) | |
| (reconstituted) | | |
| Necator americanus | XP_013295720.1 | Group II intron (eukaryotic) |
| Axinella verrucosa | CRX66588.1 | Group II intron (eukaryotic) |
| Axinella verrucosa | CRX66589.1 | Group II intron (eukaryotic) |
| Xenopolymerase RTX | Jared W. Ellefson, | Thermococcus kodakarensis |
| | et. al (2016) | (engineered) |

*Geobacillus stearothermophilus GsI-IIC intron RT (denoted GsI-IIC RT; sold commercially as TGIRT-III; InGex); see Stamos et al., Mol Cell. 2017 Dec. 7; 68(5): 926-939.e4.

Exemplary RT sequences include:

Eubacterium rectale RT (aka Marathon-RT; WT)
(SEQ ID NO: 76)
MDTSNLMEQILSSDNLNRAYLQVVRNKGAEGVDGMKYTELKEHL
AKNGETIKGQLRTRKYKPQPARRVEIPKPDGGVRNLGVPTVTDR
FIQQAIAQVLTPIYEEQFHDHSYGFRPNRCAQQAILTALNIMND
GNDWIVDIDLEKFFDTVNHDKLMTLIGRTIKDGDVISIVRKYLV
SGIMIDDEYEDSIVGTPQGGNLSPLLANIMLNELDKEMEKRGLN
FVRYADDCIIMVGSEMSANRVMRNISRFIEEKLGLKVNMTKSKV
DRPSGLKYLGFGFYFDPRAHQFKAKPHAKSVAKFKKRMKELTCR
SWGVSNSYKVEKLNQLIRGWINYFKIGSMKTLCKELDSRIRYRL
RMCIWKQWKTPQNQEKNLVKLGIDRNTARRVAYTGKRIAYVCNK
GAVNVAISNKRLASFGLISMLDYYIEKCVTC
Human endogenous retrovirus K consensus
(HERV-Kcon) RT
(SEQ ID NO: 77)
MKSRKRRNRVSFLGAATVEPPKPIPLTWKTEKPVWVNQWPLPKQ
KLEALHLLANEQLEKGHIEPSFSPWNSPVFVIQKKSGKWRMLTD
LRAVNAVIQPMGPLQPGLPSPAMIPKDWPLIIIDLKDCFFTIPL
AEQDCEKFAFTIPAINNKEPATRFQWKVLPQGMLNSPTICQTFV
GRALQPVREKFSDCYIIHYIDDILCAAETKDKLIDCYTFLQAEV
ANAGLAIASDKIQTSTPFHYLGMQIENRKIKPQKIEIRKDTLKT
LNDFQKLLGDINWIRPTLGIPTYAMSNLFSILRGDSDLNSKRML
TPEATKEIKLVEEKIQSAQINRIDPLAPLQLLIFATAHSPTGII
IQNTDLVEWSFLPHSTVKTFTLYLDQIATLIGQTRLRIIKLCGN
DPDKIVVPLTKEQVRQAFINSGAWQIGLANFVGIIDNHYPKTKI
FQFLKLTTWILPKITRREPLENALTVFTDGSSNGKAAYTGPKER
VIKTPYQSAQRAELVAVITVLQDFDQPINIISDSAYVVQATRDV
ETALIKYSMDDQLNQLFNLLQQTVRKRNFPFYITHIRAHTNLPG
PLTKANEQADLLVSSALIKAQELHA
Geobacillus stearothermophilus GsI-IIC RT (WT)
(SEQ ID NO: 78)
MALLERILARDNLITALKRVEANQGAPGIDGVSTDQLRDYIRAH
WSTIHAQLLAGTYRPAPVRRVEIPKPGGGTRQLGIPTVVDRLIQ
QAILQELTPIFDPDFSSSSFGFRPGRNAHDAVRQAQGYIQEGYR
YVVDMDLEKFFDRVNHDILMSRVARKVKDKRVLKLIRAYLQAGV
MIEGVKVQTEEGTPQGGPLSPLLANILLDDLDKELEKRGLKFCR
YADDCNIYVKSLRAGQRVKQSIQRFLEKTLKLKVNEEKSAVDRP
WKRAFLGFSFTPERKARIRLAPRSIQRLKQRIRQLTNPNWSISM
PERIHRVNQYVMGWIGYFRLVETPSVLQTIEGWIRRRLRLCQWL
QWKRVRTRIRELRALGLKETAVMEIANTRKGAWRTTKTPQLHQA
LGKTYWTAQGLKSLTQRYFELRQG

Geobacillus stearothermophilus GsI-IIC intron RT (GsI-IIC RT) pentamutants can also be used, e.g., comprising mutations D11R/N23R/G71R/G113K/P194R (positions bolded in the sequence above.

Exemplary MMLV RT sequences include the following:

MMLV-RT pentamutant (used in classic PE2),
without NLS, starts with T (not M)
(SEQ ID NO: 79)
TLNIEDEYRLHETSKEPDVSLGSTWLSDFPQAWAETGGMGLAVR
QAPLIIPLKATSTPVSIKQYPMSQEARLGIKPHIQRLLDQGILV
PCQSPWNTPLLPVKKPGTNDYRPVQDLREVNKRVEDIHPTVPNP
YNLLSGLPPSHQWYTVLDLKDAFFCLRLHPTSQPLFAFEWRDPE
MGISGQLTWTRLPQGFKNSPTLFNEALHRDLADFRIQHPDLILL
QYVDDLLLAATSELDCQQGTRALLQTLGNLGYRASAKKAQICQK
QVKYLGYLLKEGQRWLTEARKETVMGQPTPKTPRQLREFLGKAG
FCRLFIPGFAEMAAPLYPLTKPGTLFNWGPDQQKAYQEIKQALL
TAPALGLPDLTKPFELFVDEKQGYAKGVLTQKLGPWRRPVAYLS
KKLDPVAAGWPPCLRMVAAIAVLTKDAGKLTMGQPLVILAPHAV
EALVKQPPDRWLSNARMTHYQALLLDTDRVQFGPVVALNPATLL
PLPEEGLQHNCLDILAEAHGTRPDLTDQPLPDADHTWYTDGSSL
LQEGQRKAGAAVTTETEVIWAKALPAGTSAQRAELIALTQALKM
AEGKKLNVYTDSRYAFATAHIHGEIYRRRGWLTSEGKEIKNKDE
ILALLKALFLPKRLSIIHCPGHQKGHSAEARGNRMADQAARKAA
ITETPDTSTLLIENSSP

The present compositions and methods can make use of variants as known in the art and as provided herein, e.g., MarathonRT, GsI-IIC RT, and MMLV-RT variants, e.g., PE2 MMLV RT (with D200N, T306K, W313F, T330P, L603W mutations), or MMLV or PE2 MMLV RT truncations (truncations 2, 5, and 6; Grunewald et al. Nat Biotechnol. 2023 March; 41(3):337-343), MMLV RT variants encoded in PE6a-PE6g¹⁰ (e.g. or M-MLV RT (T128N, V223Y, D200C, with or without RNaseH domain truncation of M-MLV), Gs RT or Gs RT (A16E, L37P, A123V), Tfl RT or Tfl RT (P70T, G72V, S87G, M102I, K106R, K118R, I128V, L158Q, F269L, A363V, K413E, S492N) or Tfl RT (P70T, G72V, S87G, M102I, K106R, K118R, I128V, L158Q, F269L, A363V, K413E, S492N, K118R, S188K, I260L, S297Q, R288Q), Ec48 RT or Ec48 RT (E60K, K87E, E165D, D243N, R267I, E279K, K318E, K343N), KORV RT, WMSV RT, AVIRE RT, BAEMV RT, PERV RT, MMTV RT, Ty3 RT, GALV RT, SRV2 RT, MPMV RT, Vp96 RT, ASLV RT), as well as RT HFV, HERV, LtrA, HERV-Kcon, Tel4c, Marathon, GsI-IIC, Ma-Int5, engineered Marathon (optionally with D14R, N26R, D74R, Ni 16K, or N197R mutations), etc.

### Recombinase

Additional effectors (such as serine or tyrosine recombinases) can be included in the present proteins and compositions (FIG. 9). For example, The PCEs, LCEs, or dual-overhang ligation approaches can be used to install a recombinase attachment site (att) at a desired position in the genome. Serine and tyrosine recombinases, either fused to a PCE/LCE or expressed in trans, integrate a DNA donor containing a corresponding recombinase attachment site and a cargo of interest at the targeted location. Serine recombinases can include BxbI, PhiC31, Pa01, BceINT, etc (including those discovered from metagenomic mining efforts as described in Ref⁵¹) and tyrosine recombinases can include Cre and Flp.

clkNA Templates

The clkNA templates used in the present compositions and methods include (i) a localization moiety, (ii) a polymerization template (PT) for use with PCEs or attachment duplex region (ADR) for use with LCEs, and (iii) a flap binding region (FBR); FIGS. 10A-B show exemplary clkNAs. In some embodiments, the clkNA templates are in the order (i)-(ii)-(iii) from 5′ to 3′, but other configurations are possible (e.g. (ii)-(iii)-(i), e.g., wherein the clkNA has a 3′ moiety (e.g., chloroalkane, etc combined with a SNAP tag) rather than an HUH).

The localization moiety is a sequence or modification that binds to the PCE or LCE, e.g., an HUH endonuclease recognition site (when the CE includes an HUH), a telomeric binding sequence (when the CE includes a TBP), biotin (when the CE includes avidin), label with O⁶-benzylguanine derivatives (when the CE includes SNAP), label with O²-benzylcytosine derivatives (when the CE includes CLIP-tag), and labeled with a chloroalkane (when the CE includes HALO-tag). RNA or DNA hairpins can also be used to localize effectors (when the CE includes an RNA or DNA binding protein, such as a phage coat protein like MCP, PCP, BoxB, or Com).

The polymerization template (PT) for use with PCEs includes a portion that encodes homology to the target genome, e.g., at least 3, 4, 5, 6, 7, 8, 9, or 10 nt long, and optionally up to 50, 100, 200, 250, or 500 nt long, and a portion that includes the edit that is at least 1 nt long.

The attachment duplex region (ADR) for use with LCEs includes a portion that encodes homology to the target genome, e.g., at least 3, 4, 5, 6, 7, 8, 9, or 10 nt long, and up to 20 or 25 nt long, and a portion that includes the edit that is at least 1 nt long. As shown in FIG. 10B, this region is double stranded, with the 5′ end of the attachment.

The flap binding region is complementary to the genomic flap released by the nickase. In general, the length of the genomic flap is the distance between the DNA nick on the NTS and equivalent NTS position that is analogous to the end of the TS/gRNA spacer, which will often be about 15-20, e.g., 17, nt but it can be target specific. In some embodiments, the flap can be shorter (e.g., in the case of truncated gRNAs (Fu et al., Nat Biotechnol. 2014 March; 32(3): 279-284) if the gRNA spacer region is shorter. In some embodiments, the flap can be longer, though such arrangements may be thermodynamically less favorable, if the TS/NTS is unpaired outside of the gRNA spacer/TS region.

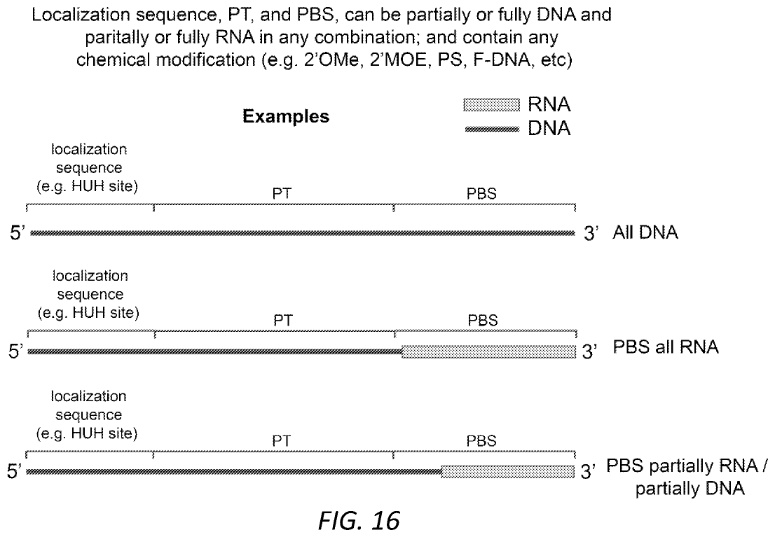

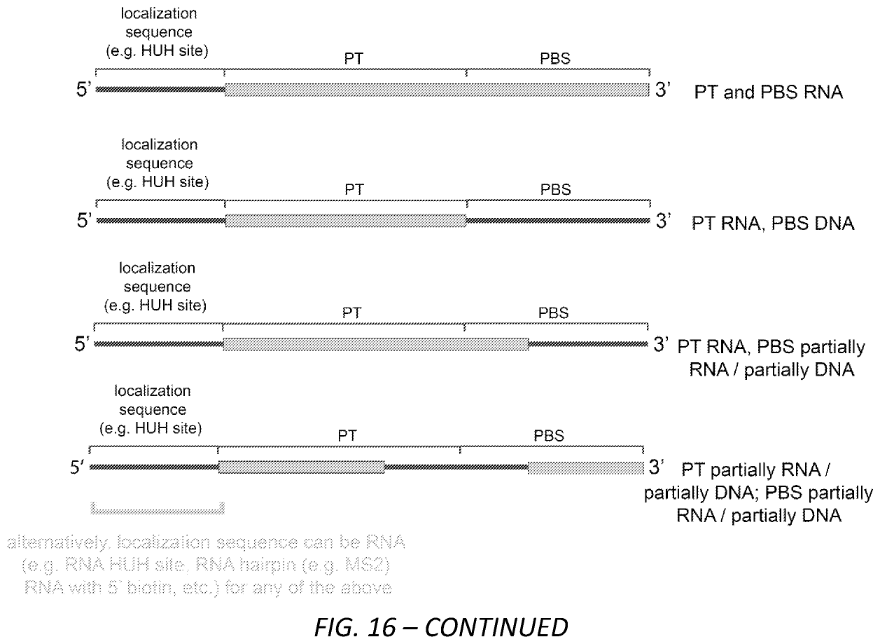

The clkNA templates can be made up of any composition of nucleobases (e.g. DNA or RNA; FIG. 16)); e.g., all DNA or partly DNA and partly RNA; HUH endonuclease sequence is DNA and the rest is RNA; HUH endonuclease sequence is DNA, all or a portion of the FBR is RNA and the PT is DNA; HUH endonuclease sequence is DNA, and all or a portion of the FBR is RNA and all or a portion of the PT is RNA. Additional DNA or RNA sequences can be added to the 3′ end of the clkNAs that is not the FBR sequence.

In addition, the clkNA templates can have one or more chemical modifications (e.g., 2′ Fluoro, 2′-F-ANA 2′OMe, 2′MOE, exNAs, PS linkages, morpholinos, locked nucleic acids (LNAs), bridged nucleic acids (BNAs), inverted bases, extended nucleic acids (exNAs), etc.). For example, the clkNA can comprise one or more modifications comprising: a modified sugar moiety, and/or a modified internucleoside linkage, and/or a modified nucleotide and/or combinations thereof. It is not necessary for all positions in a given oligonucleotide to be uniformly modified, and more than one of the modifications described herein can be incorporated in a single oligonucleotide or even at within a single nucleoside within an oligonucleotide.

In some embodiments, the clkNA templates are chimeric oligonucleotides that contain two or more chemically distinct regions, each made up of at least one nucleotide. These clkNA templates can, e.g., contain at least one region of modified nucleotides that confers one or more beneficial properties (such as, for example, increased nuclease resistance, increased uptake into cells, increased binding affinity for the target, decreased innate immune response, decreased or ablation of RNAseH activation). Chimeric clkNA templates can be formed as composite structures of two or more oligonucleotides, modified oligonucleotides, oligonucleosides and/or oligonucleotide mimetics as described herein. Such compounds have also been referred to in the art as hybrids or gapmers.

In some embodiments, the clkNA templates comprises at least one nucleotide modified at the 2′ position of the sugar, most preferably a 2′-O-alkyl, 2′-O-alkyl-O-alkyl or 2′-fluoro-modified nucleotide. In other preferred embodiments, RNA modifications include 2′-fluoro, 2′-amino and 2′ O-methyl modifications on the ribose of pyrimidines, abasic residues or an inverted base at the 3′ end of the RNA. Such modifications are routinely incorporated into oligonucleotides and these oligonucleotides have been shown to have a higher Tm (i.e., higher target binding affinity) than; 2′-deoxyoligonucleotides against a given target.

A number of nucleotide and nucleoside modifications have been shown to make oligonucleotides into which they are incorporated more resistant to nuclease digestion than the native oligodeoxynucleotide; these modified oligos survive intact for a longer time than unmodified oligonucleotides. Specific examples of modified oligonucleotides include those comprising modified backbones, for example, phosphorothioates, phosphotriesters, extended nucleic acid (exNA) (WO2021195533), methyl phosphonates, short chain alkyl or cycloalkyl intersugar linkages or short chain heteroatomic or heterocyclic intersugar linkages. Most preferred are oligonucleotides with phosphorothioate backbones and those with heteroatom backbones, particularly CH₂—NHO—CH₂, CH, ~N(CH₃)~O~CH₂ (known as a methylene(methylimino) or MMI backbone], CH₂—O—N(CH₃)—CH₂, CH₂—N(CH₃)—N(CH₃)—CH₂ and O—N(CH₃)—CH₂—CH₂ backbones, wherein the native phosphodiester backbone is represented as O—P—O—CH,); amide backbones (see De Mesmaeker et al. Ace. Chem. Res. 1995, 28:366-374); morpholino backbone structures (see Summerton and Weller, U.S. Pat. No. 5,034,506); peptide nucleic acid (PNA) backbone (wherein the phosphodiester backbone of the oligonucleotide is replaced with a polyamide backbone, the nucleotides being bound directly or indirectly to the aza nitrogen atoms of the polyamide backbone, see Nielsen et al., Science 1991, 254, 1497). Phosphorus-containing linkages include, but are not limited to, phosphorothioates, chiral phosphorothioates, phosphorodithioates, phosphotriesters, aminoalkylphosphotriesters, methyl and other alkyl phosphonates comprising 3′alkylene phosphonates and chiral phosphonates, phosphinates, phosphoramidates comprising 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′.

Morpholino-based oligomeric compounds are described in Dwaine A. Braasch and David R. Corey, Biochemistry, 2002, 41(14), 4503-4510); Genesis, volume 30, issue 3, 2001; Heasman, J., Dev. Biol., 2002, 243, 209-214; Nasevicius et al., Nat. Genet., 2000, 26, 216-220; Lacerra et al., Proc. Natl. Acad. Sci., 2000, 97, 9591-9596; and U.S. Pat. No. 5,034,506, issued Jul. 23, 1991. In some embodiments, the morpholino-based oligomeric compound is a phosphorodiamidate morpholino oligomer (PMO) (e.g., as described in Iverson, Curr. Opin. Mol. Ther., 3:235-238, 2001; and Wang et al., J. Gene Med., 12:354-364, 2010; the disclosures of which are incorporated herein by reference in their entireties).

### Polymerase Click Editors (PCEs)

The use of DNA-dependent DNA polymerases to ‘write’ DNA edits directly into endogenous genomic sites offers advantages over prior genome engineering approaches. The localization of a ssDNA template (bearing mutations of interest) to a nickase-bound target site could enable direct writing of virtually any small edit onto the 3′ end of the nicked DNA (via the DNA-polymerase acting on the localized template). Unlike PEs that utilize RT domains to polymerize nascent DNA bases from an RNA template, we envisioned that DNA-dependent DNA polymerases may offer several advantages when installing DNA edits (in terms of efficiency, accuracy, and processivity). Encoding the desired edit on a separate DNA template (clkNA or clkDNA) rather than on an RNA template (pegRNA) has benefits related to edit purity (e.g., no read-through into the pegRNA scaffold), cost and accuracy of nucleic acid synthesis, and scalability of template optimization by bypassing the requirement for cloning different pegRNAs. The clkNA can be provided in trans, can be recruited via covalent or non-covalent nucleic acid binding domains, or can be recruited through the gRNA, all with the goal of maximizing the local concentration of the clkNA at the PCE target site to enhance DNA polymerization.

We explored various PCE construct architectures including the fusion or recruitment of DNA-dependent DNA polymerase domains to a nickase Cas9 capable of nicking the NTS (HNH-inactive). One potential method for recruiting the clkNA to the target site is to utilize HUH endonucleases (Table A), which can covalently localize a single-stranded clkNA, containing an edit of interest, to a genomic site for target-primed polymerization (along with a gRNA, comprising first generation PCEs called PCE1). HUH endonucleases are small, sequence-specific enzymes that form covalent adducts with single-stranded DNA (ssDNA) via 5′-phosphotyrosine formation (FIG. 1a). They are found in a vast diversity of bacteria and archaea as well as plant and mammalian viruses⁵² and hold diverse roles in replication, conjugation, transposition, and recombination. The previous use of “HUH tags” has harnessed these enzymes as a versatile bioconjugation platform to covalently tether ssDNA to proteins⁵³. These HUH tags are employed in a variety of biotechnology applications, such as receptor specific adeno-associated virus (AAV) cell targeting⁵⁴, DNA origami-based protein assembly^{55,57}, nanoparticle drug delivery⁵⁸, live cell imaging⁵³, and improved CRISPR-Cas9 mediated HDR, through DNA donor localization to the site of the DNA DSB⁵⁹. The HUH endonuclease PCV2 and its homologs have been shown to be as active as the highly engineered, commercially available SNAP tag—which can similarly form a phosphotyrosine adduct—without requiring expensive ssDNA chemical modifications⁵³. Despite this proven platform for protein-ssDNA conjugation, HUH endonucleases are unexplored for next-generation genome editing technologies that minimize DNA DSBs and are independent of HDR. As noted above, as an alternative to HUH endonucleases, there are other nucleic acid recruitment strategies that will also enable recruitment of the clkNA to the PCE target site (e.g., avidin variants (when the clkNA is labeled with biotin), SNAP-tag (when the clkNA is labeled with O⁶-benzylguanine derivatives), CLIP-tag (when the clkNA is labeled with O²-benzylcytosine derivatives), and HALO-tag (when the clkNA is labeled with a chloroalkane)) or when the clkNA comprises an RNA motif that is bound by an RNA binding protein, e.g., a phage coat protein (CP), or a phage antitermination signal, such as MCP, MCP(N55K), PCP, Com, Phi21 N protein (N^{Phi22})⁸, Phi 22 N protein (N^{Phi22}), lambda N protein (N^{lambda}), or an evolved RNA-binding HUH endonuclease⁹ (exemplary sequences in Table B).

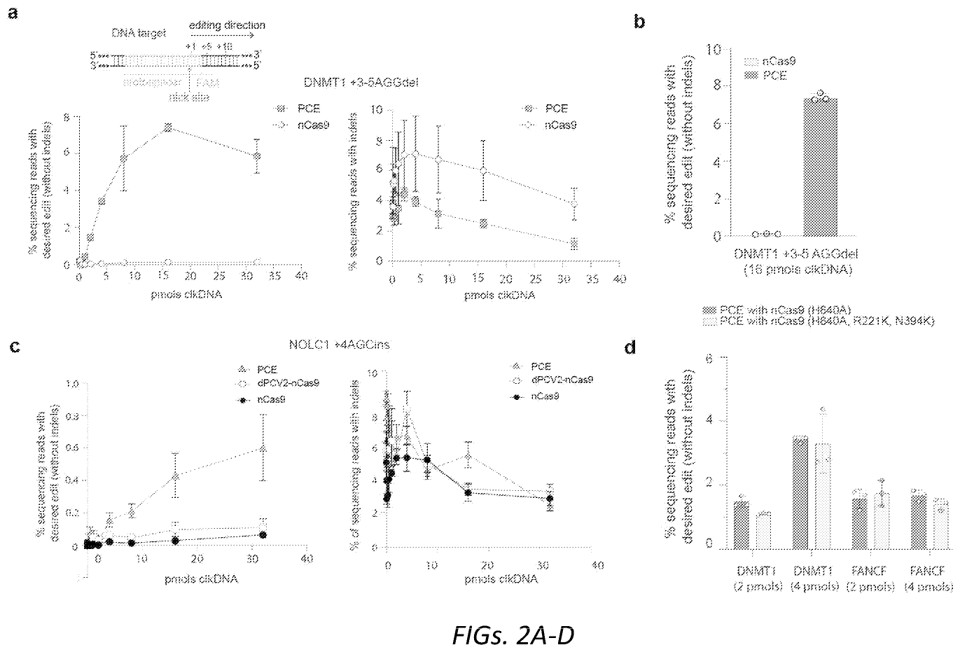

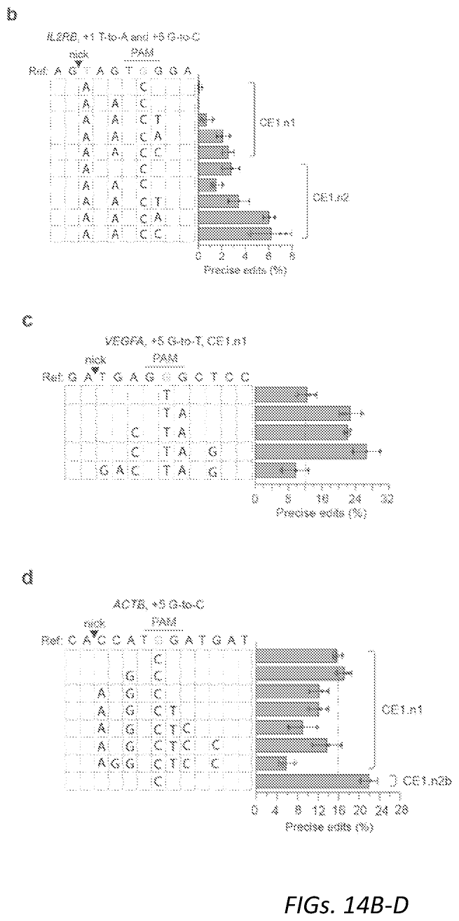

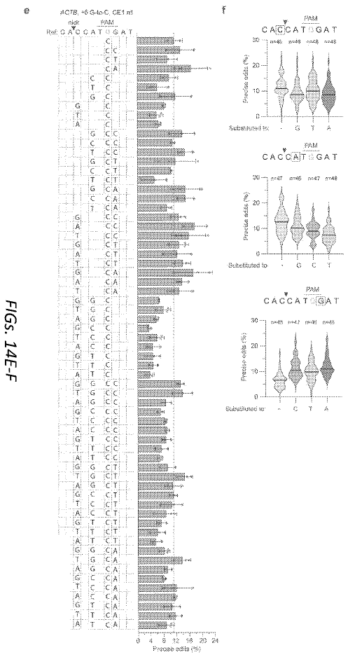

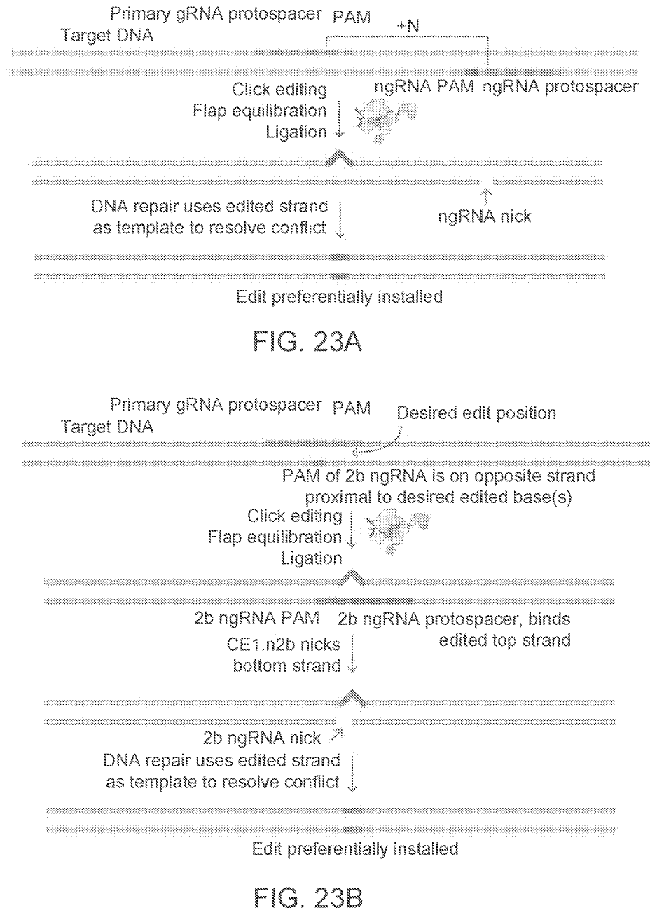

Without wishing to be bound by theory, it is believed that once the PCE and clkNA are bound at the target site, hybridization of the Flap Binding Region (FBR) of the clkNA to the 3′ NTS DNA flap released by nCas9 creates a genomic primer for DNA-dependent DNA polymerase to directly write in the edit(s) of interest from the polymerization template (PT; FIG. 1b). The PCE enzyme—clkNA complex then dissociates from the target site, permitting flap equilibration between the nascent edit-encoding 3′-flap and the genomic DNA 5′-flap. Then, cleavage of the 5′ endogenous flap and nick ligation leads to installation of the edit of interest into the genomic site. To enhance incorporation of the edit, a secondary gRNA can be delivered that when complexed with the nCas9 PCE will create a separate nick on the TS DNA, thereby biasing MMR towards edit incorporation (methods that use a second gRNA are termed PCE2) (FIG. 2B, FIG. 23). Furthermore, the use of secondary gRNAs that direct a nick to the non-edited strand only after edit installation (after 5′ flap cleavage and nick ligation) should decrease DNA DSBs and indel generation by preventing concurrent nearby nicks, while also offering advantages for overall efficiency (we denote the secondary gRNA strategy that is dependent on the presence of the edit as PCE2b; FIG. 1C, FIG. 23B). In some embodiments, the PCE2 (secondary) gRNAs are at least 5-10 nts and up to 50, 100, 150, 200, or more nts, away from the primary PCE target site. Beyond exploring alternate polymerases, additional modifications to PCEs that may improve edit efficiency and edit purity include the: (1) PCE architecture (e.g. identity of the HUH endonuclease (e.g., PCV2, etc.; Table A), identity of the DNA binding domain (e.g. various nCas9 or Cas9 orthologs, nCas12a or Cas12 orthologs, nIscB or IscB orthologs, nTnp or Tnp orthologs, etc.; Tables C, D, and E), different amino acid linkers between protein domains (of various lengths and configurations, e.g. GSG, GGSGGSGG (SEQ ID NO: 80), (GGS)n (SEQ ID NO: 81), (AP)n (SEQ ID NO: 82), (EAAAK)n (SEQ ID NO: 83), SGSETPGTSESATPES (SEQ ID NO: 84), SGGSSGSETPGTSESATPESSGGS (SEQ ID NO: 85), etc.), codon optimization of the PCE or LCE construct, other methods to recruit the clkNA to the PCE beyond HUH enzymes (see, e.g., Table B), other domains to recruit the polymerase to nCas9 via protein mediated tethering (e.g. CC, LZ, or SunTag domains) expression of the polymerase in trans instead of fusion, etc.; FIGS. 10A-G), (2) clkNA properties (chemical modifications, hairpin structures at the 5′ or 3′ ends, etc.), (3) clkNA sequence designs (FBR and PT lengths, etc)¹⁴, and/or (4) DNA repair modulation^{60,61} (transient MLH1 or TREX1 knockdown, MLH1dn co-expression, encoded silent mutations in the clkNA, etc.), which may also improve PCE editing outcomes and efficiency (FIGS. 14A-F).

### Double Click

Given the ability of PCEs to directly write new sequences onto 3′ genomic flaps, we imagine that PCEs can be harnessed for more complex larger sequence edits. PCEs should in principle be able to install larger sequence edits by replacing the sequence between two distal genomic flaps, where subsequent annealing of these flaps may lead to efficient exchange of the intervening genetic sequence (and having the benefit of bypassing the DNA repair-dependent steps required for canonical PCEs).

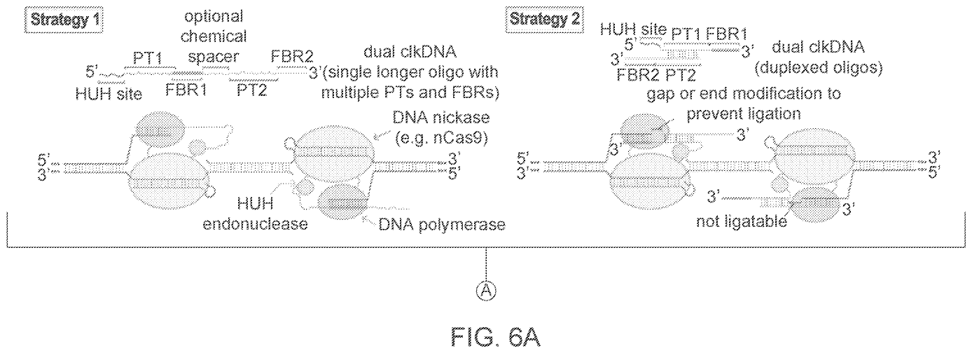

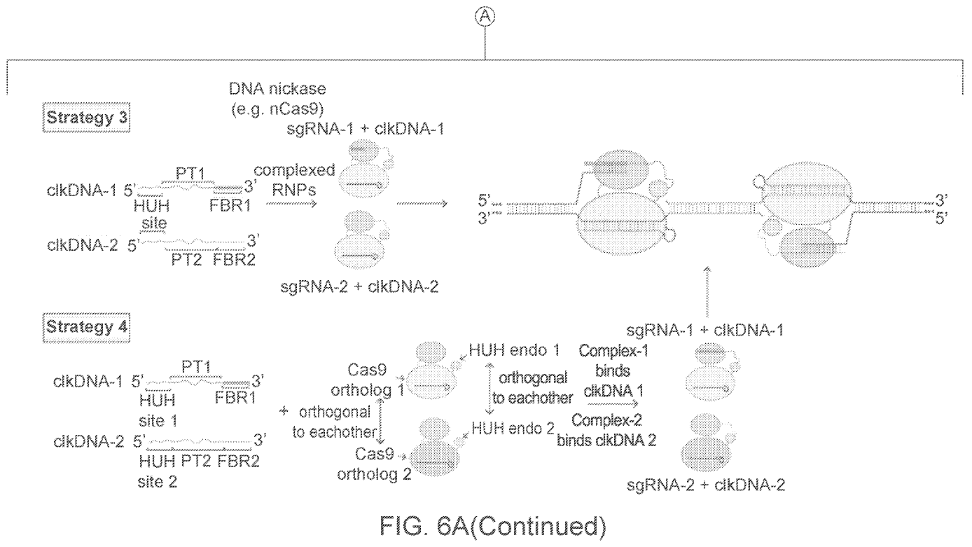

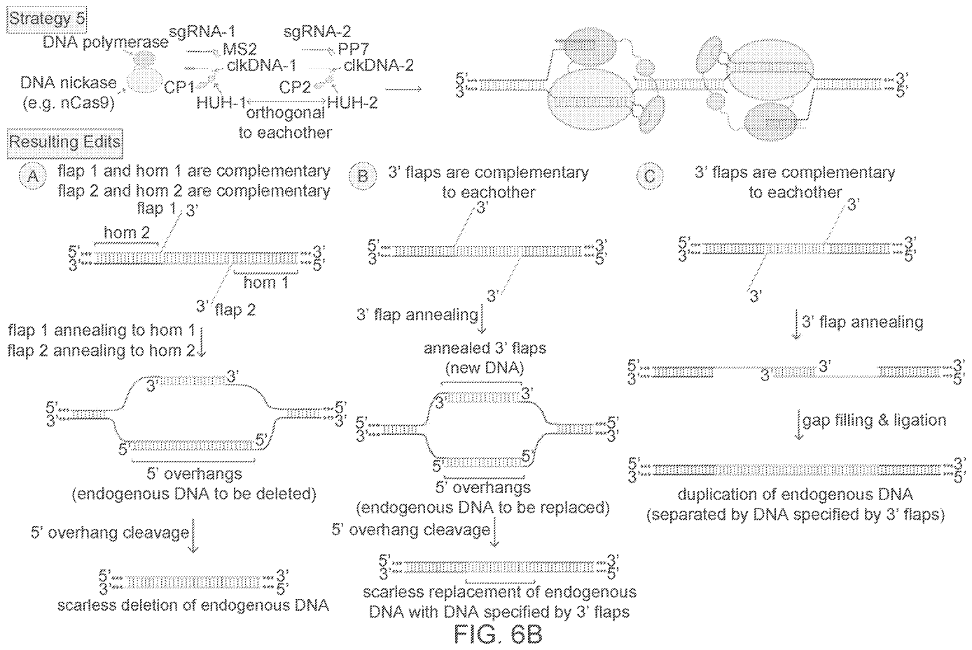

More specifically, a pair of sgRNAs—each targeting opposite DNA strands—could enable two 3′ flaps to be generated simultaneously at the target site, resulting in various edits. For example:
Deletion: each of the two 3′ flaps is complementary to the upstream sequence of the other nick, resulting in flap-templated DNA repair and subsequent deletion of the sequence between the two nicks.
Replacement: the two 3′ flaps are complementary to each other and placed downstream of each other, resulting in 3′ flap annealing. DNA repair (cleavage of the 5′ overhangs) results in replacement of genomic sequence between the two nicks with the sequence contained in the annealed 3′ flaps (FIGS. 40A, B).
Duplication: the two 3′ flaps are complementary to each other and placed upstream of each other, resulting in 3′ flap annealing. DNA repair (cleavage of the 5′ overhangs) and DNA synthesis results in duplication of the genomic sequence between the two nicks separated by the sequence contained within the 3′ flaps.
   Thus, these approaches would enable targeted and precise deletion, replacement, and duplication of endogenous DNA sequences (FIG. 6). We term this approach “Double Click”.

Double click requires that the correct sgRNA-Cas RNP associates with the correct corresponding clkNA sequence containing the appropriate FBR and PT to ensure that the FBR can hybridize to the genomic flap and that the PT-defined 3′ flap can be installed at the correct location. If the incorrect pair is formed, the FBR will not hybridize with the genomic flap (no or limited complementarity), and therefore, no polymerization will occur. Moreover, if the incorrect flap is installed at either nick site, the intended edit will not occur. To ensure this correct RNP-clkNA pairing, the clkNA can be designed in multiple ways (FIG. 6): (1) a single long ‘dual’ clkNA containing two FBRs and two PTs, optionally with a chemical linker between the end of FBR1 and beginning of PT2 (2) a duplexed oligo containing two 3′ overhangs corresponding to an FBR at one of the sites defined by each sgRNA (3) separated clkNAs, each having an FBR and PT, which can be precomplexed with the correct sgRNA-Cas RNP. (4) Orthogonal HUH enzymes and RNA-programmed nickases can also be used (for example, to enable in cellulo packaging or one-pot in vitro RNP formation). (5) Orthogonal HUH enzymes fused to orthogonal recruitment domains (e.g. coat proteins, MCP and PCP) can be used to localize a prescribed clkNA to the proper sgRNA at the Cas9-bound target site, where each sgRNA contains the appropriate RNA-recruitment hairpin (e.g., MS2 and PP7 for MCP and PCP, respectively). Alternatively, the exemplary PCE architecture which fuses the recruitment domain (e.g. HUH endonuclease) and the DNA polymerase with the DNA-binding domain separate (e.g. HUH-Pol or Pol-HUH+DBD) can be used—where the correct genomic flap is bound by the correct clkDNA based solely on sequence complementarity of the FBR encoded on the clkDNA (and without direct recruitment to the correct guide-containing complex).

### Ligase Click Editors (LCEs)

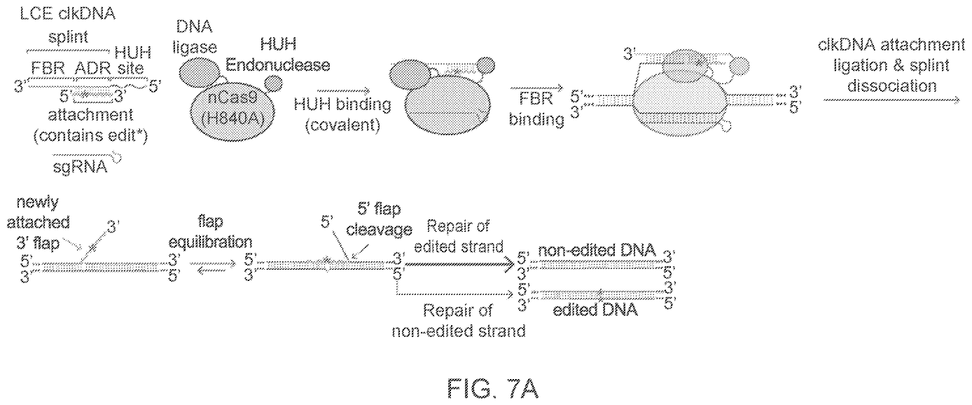

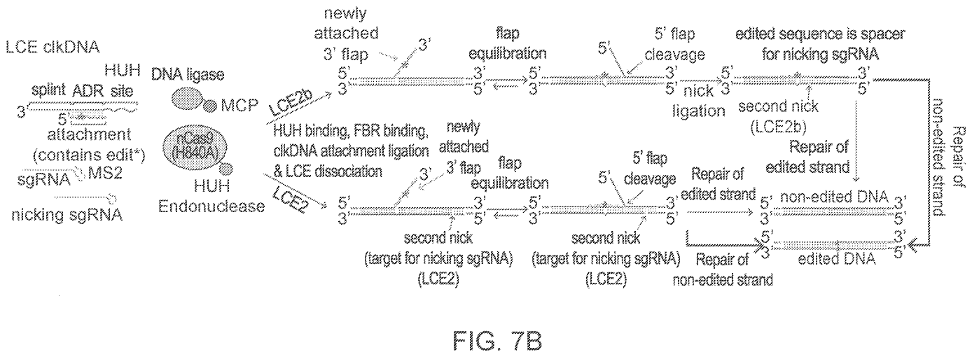

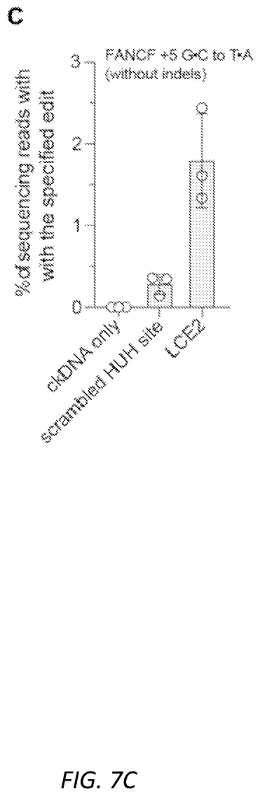

Beyond polymerization off of a clkNA template, we also hypothesized that the installation of virtually any small edit (i.e., user-defined substitutions, small insertions, and/or small deletions at a genomic target site) might also be possible via direct ligation of a DNA substrate encoding the edit of interest to the 3′ end of the nicked NTS of the nCas9 target site. To test this approach, we generated ligase click editors (LCEs) comprised of a DNA nickase to liberate the 3′ end of the NTS (i.e. in this instance, nCas9 with an H840A HNH-inactivating mutation), a clkDNA recruiting domain (e.g., in this instance, an HUH endonuclease), and a DNA ligase (i.e., in this instance, T4 DNA ligase). We term this system LCE version 1 (LCE1). We envisioned that certain components in this system may also be recruited to nCas9 via other methods or expressed in trans rather than directly fused to nCas9. As described above, HUH endonucleases have advantages for recruiting ssDNA templates to proteins, given that they are small, sequence-specific enzymes that form covalent adducts with ssDNA templates that do not require expensive chemical modification (FIG. 7a).

In the case of LCEs, as one example a clkNA can be used that is comprised of two DNA strands that can be formed by annealing two DNA oligonucleotides (FIG. 7A). The first DNA strand has three components, including (1) the 5′ region that encodes the HUH endonuclease recognition sequence, (2) an Attachment Duplex Region (ADR) that is complementary to an attachment sequence (described below) and (3) a 3′ region that is complementary to the accessible flap of the nicked NTS DNA (of the nCas9 target site, analogous to the FBR of PCE clkNAs; FIG. 1B, 7A). The second strand of the clkNA, called the “attachment sequence”, is complementary to the ADR of the primary clkNA strand and encodes the edit(s) of interest. The attachment sequence serves as the substrate for direct DNA:DNA ligation on to the 3′ end of the nicked NTS. Ligation of the attachment to the genomic flap would result in an extended 3′ flap (containing the edit) that would undergo flap equilibration (with the competing endogenous 5′ flap sequence), cleavage and removal of the 5′ endogenous flap, and nick ligation. This mechanism would result in permanent installation of the edit(s) into the genome with high precision and without a reliance on DSBs, HDR, or other DNA repair pathways (FIG. 7A). It is also possible that the primary strand of the LCE clkNA may act as a template for endogenous polymerases to write in the specified edit(s) (analogous to the PCE/clkNA mechanism) depending on the kinetics and activity of the cellular DNA ligases and/or the ligase fused to the LCE construct.

### LCE2

We also developed a strategy that we termed LCE2, which employs a second sgRNA to subvert DNA repair by creating a nick at a separate target site on the non-edited strand (FIG. 7B) analogous to the mechanisms of other systems (BEs, PEs, PCEs). To avoid recruiting the ligase to this secondary nick (which would seal the nick and diminish the benefit of the second strand nick), the ligase is recruited to the primary target site only and not the secondary site (FIG. 7B). As noted above, one method to selectively recruit proteins or domains to specific target sites is to fuse the effector protein-of-interest (e.g., ligase or polymerase) to a recruitment domain (e.g., an RNA binding protein, e.g., MCP, PCP, or Com RNA binding protein^{37,38}) in this instance, the MS2-coat protein (MCP); FIG. 7B) that then interacts with a specific hairpin sequence encoded within that gRNA (e.g., viral RNA sequences MS2, PP7, and com, in this instance, an MS2 hairpin). This permits selective recruitment of the effector to the site bound by the primary gRNA; the gRNA targeting the secondary nicking site would not harbor the MS2 hairpin, preventing recruitment to that site (FIG. 7B). The design of secondary nicking target sites to target the non-edited strand only after edit installation and flap resolution of the edited strand (cleavage of the 5′ flap and nick ligation) should also decrease insertion or deletion mutations (indels) by preventing concurrent nicks, as well as temporal control when the ligase is localized to the genomic site (FIG. 6B). We named this strategy LCE2b.

### Dual-Overhang Ligation

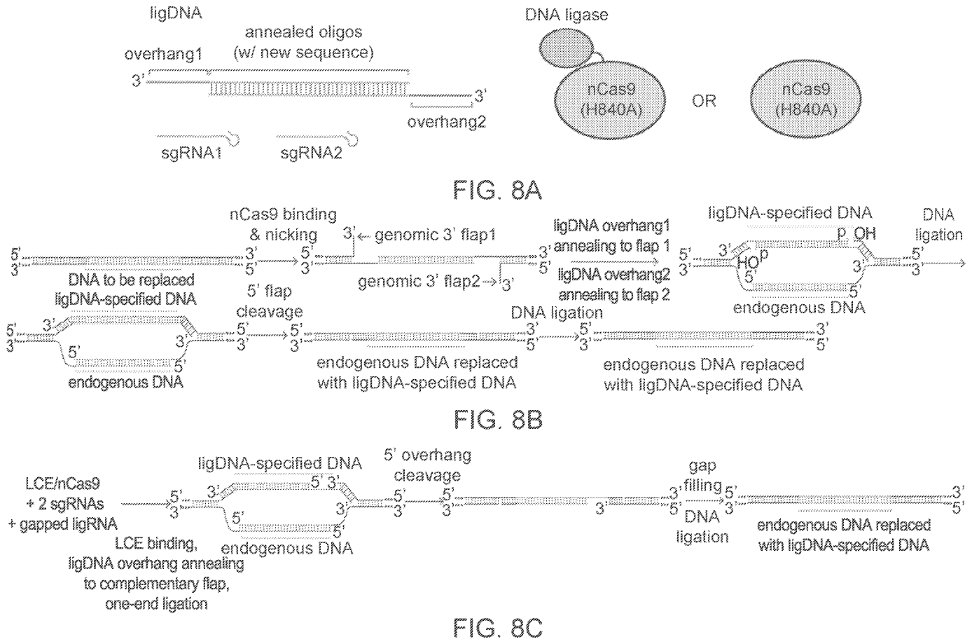

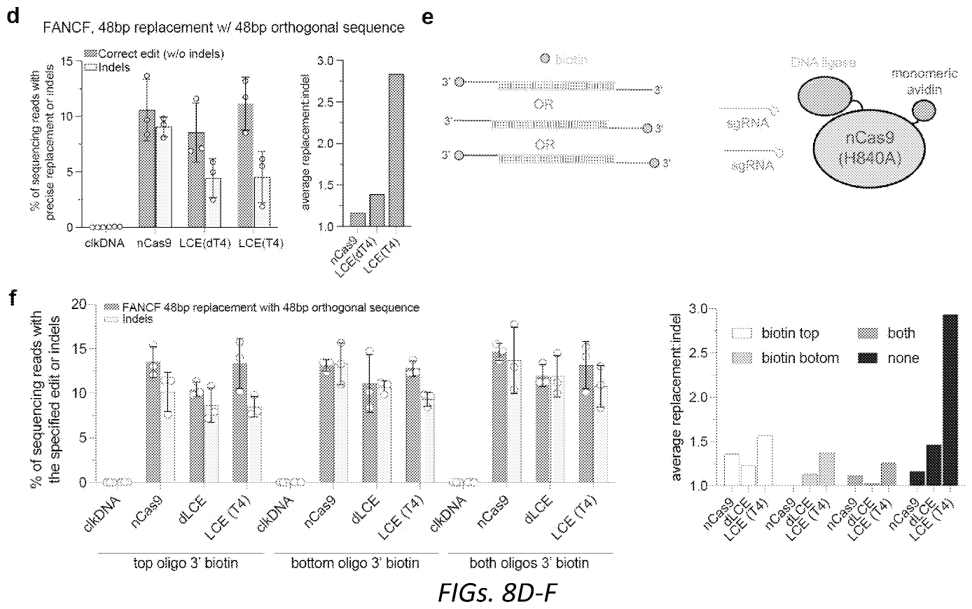

As an alternative to Double Click, and in addition to the original LCE1/2/2b methods, we have developed a ligation-based approach to enable precise replacement of endogenous DNA. Two sgRNAs are used to target two sites can be designed in a 5′ top strand >3′ bottom strand orientation to create sequence replacements; FIG. 8A); alternately, the target sites can be designed in a 3′ top strand >5′ bottom strand orientation to create sequence duplications. Nicking at each target site exposes a 3′ genomic flap (two total from a flap at each site). A duplexed DNA substrate containing two 3′ overhangs complementary to one or the other genomic flaps (a ligation DNA or ligDNA, as opposed to a clkNA) can anneal to the complementary genomic flap. Ligation of the nick between the genomic flap and duplexed substrate on both ends by endogenous ligases, and subsequent DNA repair (e.g. 5′ overhang cleavage) can lead to replacement of the sequence between the two nicks with the sequence defined by the ligDNA (FIG. 8A). We term this “Dual overhang ligation”.

Additionally, we hypothesized that fusing a DNA ligase could facilitate nick sealing of splinted flap-ligDNA, potentially increasing product purity by both locking the ligDNA into place as well as ligating genomic nicks remnant from unsuccessful editing events (FIG. 8A, 8B).

PCEs, LCEs, or Dual-Overhang Ligation Combined with Serine Recombinases for Gene-Sized DNA Insertions

Site-specific serine recombinases irreversibly integrate DNA sequences containing an attP attachment site into a target DNA sequence containing a corresponding attB attachment site. (Merrick J Z, et al., ACS Synth. Biol. 2018. Serine Integrases: Advancing Synthetic Biology; Rutherford K, et al., Curr Opin Struct Biol. 2014. The ins and outs of serine integrase site-specific recombination), Click Editors can be used to install attB or attP at specific sites in the genome to direct programmable DNA integration into a targeted genomic site (FIG. 40B). Either the attB or attP is on the donor plasmid and the other is the edit on the clkNA; for example, if an attP is the edit on the clkNA, then the attB is on the dsDNA donor that will be integrated, or vice versa (attB as the edit and attP on the donor). Programmable gene-sized DNA integration into genomes could enable genetic medicines that can be generalized to a range of patients afflicted by a common disease, irrespective of their genetic mutation(s). Additionally, this capability could facilitate cell engineering efforts where installation of large genetic sequences at targeted locations could endow cells with new capabilities while obviating safety, efficacy, and manufacturing issues resulting from traditional random integration approaches.

To programmably and precisely integrate gene-sized DNA sequences, we hypothesized that PCEs (single-flap or double click), LCEs, or dual-overhang ligation approaches, could be leveraged to place ~30-60 bp attB or attP sequences at a desired location in a genome. A serine recombinase, such as BxbI or Pa01⁵¹, either fused to the PCE or LCE or provided in trans, could then integrate a donor DNA molecule containing the corresponding attachment site (i.e. attP if attB is genomically installed or attB if attP is genomically installed) into the genome at the RNA-programmed location (FIG. 9). For example, a donor DNA is used that has an attB or attP (to install an attB, then the donor has an attP; and vice versa). Attar and atto refers to the sites on the genome after recombination has occurred. This can be used to create deletions, inversions, and translocation. For deletions, attB and attP are installed into a genome flanking the sequence to be deleted (both facing same direction), contact with a recombinase deletes the intervening sequence. For an inversion, attB and attP are installed flanking a region to be inverted (facing inwards), and contact with a recombinase inverts the intervening sequence. For a translocation, attB is installed on one chromosome, and attP is installed on the other, and contact with a recombinase results in a translocation.

### Exemplary Sequences and Constructs

In some embodiments, the sequence of a protein or nucleic acid used in a composition or method described herein is at least 80%, 85%, 90%, 95%, 97%, 98%, or 99% identical to a reference sequence set forth herein. To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 80% of the length of the reference sequence, and in some embodiments is at least 90% or 100%. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. For example, the percent identity between two amino acid sequences can be determined using the Needleman and Wunsch ((1970) J. Mol. Biol. 48:444-453) algorithm which has been incorporated into the GAP program in the GCG software package (available on the world wide web at gcg.com), using the default parameters, e.g., a Blossum 62 scoring matrix with a gap penalty of 12, a gap extend penalty of 4, and a frameshift gap penalty of 5.

### Exemplary Sequences

Exemplary DNA ligase sequences

| name | aa sequence |
|---|---|
| T3 | MNIFNTNPFKAVSFVESAVKKALETSGYLIADCKYDGVRGNIVV |
| | DNVAEAAWLSRVSKFIPALEHLNGFDKRWQQLLNDDRCIFPDGF |
| | MLDGELMVKGVDFNTGSGLLRTKWVKRDNMGFHLTNVPTKLTP |
| | KGREVIDGKFEFHLDPKRLSVRLYAVMPIHIAESGEDYDVQNLLM |
| | PYHVEAMRSLLVEYFPEIEWLIAETYEVYDMDSLTELYEEKRAEG |
| | HEGLIVKDPQGIYKRGKKSGWWKLKPECEADGIIQGVNWGTEGL |
| | ANEGKVIGFSVLLETGRLVDANNISRALMDEFTSNVKAHGEDFY |
| | NGWACQVNYMEATPDGSLRHPSFEKFRGTEDNPQEKM |
| | (SEQ ID NO: 86) |
| T4 | MILKILNEIASIGSTKQKQAILEKNKDNELLKRVYRLTYSRGLQYY |
| | IKKWPKPGIATQSFGMLTLTDMLDFIEFTLATRKLTGNAAIEELTG |
| | YITDGKKDDVEVLRRVMMRDLECGASVSIANKVWPGLIPEQPQM |
| | LASSYDEKGINKNIKFPAFAQLKADGARCFAEVRGDELDDVRLLS |
| | RAGNEYLGLDLLKEELIKMTAEARQIHPEGVLIDGELVYHEQVK |
| | KEPEGLDFLFDAYPENSKAKEFAEVAESRTASNGIANKSLKGTISE |
| | KEAQCMKFQVWDYVPLVEIYSLPAFRLKYDVRFSKLEQMTSGY |
| | DKVILIENQVVNNLDEAKVIYKKYIDQGLEGIILKNIDGLWENARS |
| | KNLYKFKEVIDVDLKIVGIYPHRKDPTKAGGFILESECGKIKVNA |
| | GSGLKDKAGVKSHELDRTRIMENQNYYIGKILECECNGWLKSDG |
| | RTDYVKLFLPIAIRLREDKTKANTFEDVFGDFHEVTGL (SEQ ID |
| | NO: 87) |
| T7 | MNIKTNPFKAVSFVESAIKKALDNAGYLIAEIKYDGVRGNICVDN |
| | TANSYWLSRVSKTIPALEHLNGFDVRWKRLLNDDRCFYKDGFM |
| | LDGELMVKGVDFNTGSGLLRTKWTDTKNQEFHEELFVEPIRKKD |
| | KVPFKLHTGHLHIKLYAILPLHIVESGEDCDVMTLLMQEHVKNM |
| | LPLLQEYFPEIEWQAAESYEVYDMVELQQLYEQKRAEGHEGLIV |
| | KDPMCIYKRGKKSGWWKMKPENEADGIIQGLVWGTKGLANEG |
| | KVIGFEVLLESGRLVNATNISRALMDEFTETVKEATLSQWGFFSP |
| | YGIGDNDACTINPYDGWACQISYMEETPDGSLRHPSFVMFRGTE |
| | DNPQEKM (SEQ ID NO: 88) |
| ChlV | MAITKPLLAATLENIEDVQFPCLATPKIDGIRSVKQTQMLSRTFKPI |
| (SplintR) | RNSVMNRLLTELLPEGSDGEISIEGATFQDTTSAVMTGHKMYNA |
| | KFSYYWFDYVTDDPLKKYIDRVEDMKNYITVHPHILEHAQVKIIP |
| | LIPVEINNITELLQYERDVLSKGFEGVMIRKPDGKYKFGRSTLKEG |
| | ILLKMKQFKDAEATIISMTALFKNTNTKTKDNFGYSKRSTHKSGK |
| | VEEDVMGSIEVDYDGVVFSIGTGFDADQRRDFWQNKESYIGKMV |
| | KFKYFEMGSKDCPRFPVFIGIRHEEDR (SEQ ID NO: 89) |
| PhiKMV | MSKRDVVLDIEKGIWRGVDQNDKAVEAIIKKNGYVIVEPKIDGC |
| | RAIVGAHGVVSRSGRRFPALDGLEDRIIAKLSQSGLDSGLVLDCE |
| | MYLEGMPFSEATGRMARKTPLTKAELKCLHFAVFDATHIGVLRK |
| | SRKSHLVYDERRAMVRSLMEDCRRGDTPYFFQVAAQSCRSMEA |
| | VLRWYGYHRAMGFEGSMEKDPSLTYRNGKVAGCYKRKPEITVD |
| | GRIVGYVMGKTGKNVGRVVGYRVELEDGSGTVAATGLSEEHIQ |
| | LLTCAYLNAHIDEAMPNYGRIVEVSAMERSANTLRHPSFSRFRDL |
| | ASNPGVKV (SEQ ID NO: 90) |
| Vaccinia | MTSLREFRKLCCDIYHASGYKEKSKLIRDFITDRDDKYLIIKLLLP |
| | GLDDRIYNMNDKQIIKLYSIIFKQSQEDMLQDLGYGYIGDTIRTFF |
| | KENTEIRPRDKSILTLEDVDSFLTTLSSVTKESHQIKLLTDIASVCT |
| | CNDLKCVVMLIDKDLKIKAGPRYVLNAISPNAYDVFRKSNNLKEI |
| | IENASKQNLDSISISVMTPINPMLAESCDSVNKAFKKFPSGMFAEV |
| | KYDGERVQVHKNNNEFAFFSRNMKPVLSHKVDYLKEYIPKAFK |
| | KATSIVLDSEIVLVDEHNVPLPFGSLGIHKKKEYKNSNMCLFVFD |
| | CLYFDGFDMTDIPLYERRSFLKDVMVEIPNRIVFSELTNISNESQLT |
| | DVLDDALTRKLEGLVLKDINGVYEPGKRRWLKIKRDYLNEGSM |
| | ADSADLVVLGAYYGKGAKGGIMAVFLMGCYDDESGKWKTVTK |
| | CSGHDDNTLRVLQDQLTMVKINKDPKKIPEWLVVNKIYIPDFVVE |
| | DPKQSQIWEISGAEFTSSKSHTANGISIRFPRFTRIREDKTWKESTH |
| | LNDLVNLTKS (SEQ ID NO: 91) |
| dT4 | MILKILNEIASIGSTKQKQAILEKNKDNELLKRVYRLTYSRGLQYY |
| | IKKWPKPGIATQSFGMLTLTDMLDFIEFTLATRKLTGNAAIEELTG |
| | YITDGKKDDVEVLRRVMMRDLECGASVSIANKVWPGLIPEQPQM |
| | LASSYDEKGINKNIKFPAFAQLAADGAACFAEVRGDELDDVRLLS |
| | RAGNEYLGLDLLKEELIKMTAEARQIHPEGVLIDGELVYHEQVK |
| | KEPEGLDFLFDAYPENSKAKEFAEVAESRTASNGIANKSLKGTISE |
| | KEAQCMKFQVWDYVPLVEIYSLPAFRLKYDVRFSKLEQMTSGY |
| | DKVILIENQVVNNLDEAKVIYKKYIDQGLEGIILKNIDGLWENARS |
| | KNLYKFKEVIDVDLKIVGIYPHRKDPTKAGGFILESECGKIKVNA |
| | GSGLKDKAGVKSHELDRTRIMENQNYYIGKILECECNGWLKSDG |
| | RTDYVKLFLPIAIRLREDKTKANTFEDVFGDFHEVTGL (SEQ ID |
| | NO: 92) |

Exemplary DNA Polymerase sequences

| name | aa sequence |
|---|---|
| E. coli | MVISYDNYVTILDEETLKAWIAKLEKAPVFAFATATDSLDNISAN |
| Klenow | LVGLSFAIEPGVAAYIPVAHDYLDAPDQISRERALELLKPLLEDEK |
| (D355A, | ALKVGQNLKYDRGILANYGIELRGIAFDTMLESYILNSVAGRHD |
| E357A) | MDSLAERWLKHKTITFEEIAGKGKNQLTFNQIALEEAGRYAAED |
| | ADVTLQLHLKMWPDLQKHKGPLNVFENIEMPLVPVLSRIERNGV |
| | KIDPKVLHNHSEELTLRLAELEKKAHEIAGEEFNLSSTKQLQTILF |
| | EKQGIKPLKKTPGGAPSTSEEVLEELALDYPLPKVILEYRGLAKLK |
| | STYTDKLPLMINPKTGRVHTSYHQAVTATGRLSSTDPNLQNIPVR |
| | NEEGRRIRQAFIAPEDYVIVSADYSQIELRIMAHLSRDKGLLTAFA |
| | EGKDIHRATAAEVFGLPLETVTSEQRRSAKAINFGLIYGMSAFGL |
| | ARQLNIPRKEAQKYMDLYFERYPGVLEYMERTRAQAKEQGYVE |
| | TLDGRRLYLPDIKSSNGARRAAAERAAINAPMQGTAADIIKRAMI |
| | AVDAWLQAEQPRVRMIMQVHDELVFEVHKDDVDAVAKQIHQL |
| | MENCTRLDVPLLVEVGSGENWDQAH (SEQ ID NO: 93) |
| Taq Stoffel | MALEEAPWPPPEGAFVGFVLSRKEPMWADLLALAAARGGRVHR |
| | APEPYKALRDLKEARGLLAKDLSVLALREGLGLPPGDDPMLLAY |
| | LLDPSNTTPEGVARRYGGEWTEEAGERAALSERLFANLWGRLEG |
| | EERLLWLYREVERPLSAVLAHMEATGVRLDVAYLRALSLEVAEE |
| | IARLEAEVFRLAGHPFNLNSRDQLERVLFDELGLPAIGKTEKTGK |
| | RSTSAAVLEALREAHPIVEKILQYRELTKLKSTYIDPLPDLIHPRTG |
| | RLHTRFNQTATATGRLSSSDPNLQNIPVRTPLGQRIRRAFIAEEGW |
| | LLVALDYSQIELRVLAHLSGDENLIRVFQEGRDIHTETASWMFGV |
| | PREAVDPLMRRAAKTINFGVLYGMSAHRLSQELAIPYEEAQAFIE |
| | RYFQSFPKVRAWIEKTLEEGRRRGYVETLFGRRRYVPDLEARVK |
| | SVREAAERMAFNMPVQGTAADLMKLAMVKLFPRLEEMGARML |
| | LQVHDELVLEAPKERAEAVARLAKEVMEGVYPLAVPLEVEVGIG |
| | EDWLSAKE (SEQ ID NO: 94) |
| PE2 | MTLNIEDEYRLHETSKEPDVSLGSTWLSDFPQAWAETGGMGLAV |
| MMLV RT | RQAPLIIPLKATSTPVSIKQYPMSQEARLGIKPHIQRLLDQGILVPC |
| | QSPWNTPLLPVKKPGTNDYRPVQDLREVNKRVEDIHPTVPNPYN |
| | LLSGLPPSHQWYTVLDLKDAFFCLRLHPTSQPLFAFEWRDPEMGI |
| | SGQLTWTRLPQGFKNSPTLFNEALHRDLADFRIQHPDLILLQYVD |
| | DLLLAATSELDCQQGTRALLQTLGNLGYRASAKKAQICQKQVKY |
| | LGYLLKEGQRWLTEARKETVMGQPTPKTPRQLREFLGKAGFCRL |
| | FIPGFAEMAAPLYPLTKPGTLFNWGPDQQKAYQEIKQALLTAPAL |
| | GLPDLTKPFELFVDEKQGYAKGVLTQKLGPWRRPVAYLSKKLDP |
| | VAAGWPPCLRMVAAIAVLTKDAGKLTMGQPLVILAPHAVEALV |
| | KQPPDRWLSNARMTHYQALLLDTDRVQFGPVVALNPATLLPLPE |
| | EGLQHNCLDILAEAHGTRPDLTDQPLPDADHTWYTDGSSLLQEG |
| | QRKAGAAVTTETEVIWAKALPAGTSAQRAELIALTQALKMAEGK |
| | KLNVYTDSRYAFATAHIHGEIYRRRGWLTSEGKEIKNKDEILALL |
| | KALFLPKRLSIIHCPGHQKGHSAEARGNRMADQAARKAAITETPD |
| | TSTLLIENSSP (SEQ ID NO: 95) |
| Pol-Beta | MSKRKAPQETLNGGITDMLTELANFEKNVSQAIHKYNAYRKAAS |
| | VIAKYPHKIKSGAEAKKLPGVGTKIAEKIDEFLATGKLRKLEKIRQ |
| | DDTSSSINFLTRVSGIGPSAARKFVDEGIKTLEDLRKNEDKLNHHQ |
| | RIGLKYFGDFEKRIPREEMLQMQDIVLNEVKKVDSEYIATVCGSF |
| | RRGAESSGDMDVLLTHPSFTSESTKQPKLLHQVVEQLQKVHFITD |
| | TLSKGETKFMGVCQLPSKNDEKEYPHRRIDIRLIPKDQYYCGVLY |
| | FTGSDIFNKNMRAHALEKGFTINEYTIRPLGVTGVAGEPLPVDSE |
| | KDIFDYIQWKYREPKDRSE (SEQ ID NO: 96) |
| Pol-Beta + | MSKRKAPQETLNGGITDMLTELANFEKNVSQAIHKYNAYRKAAS |
| Sso7d | VIAKYPHKIKSGAEAKKLPGVGTKIAEKIDEFLATGKLRKLEKIRQ |
| | DDTSSSINFLTRVSGIGPSAARKFVDEGIKTLEDLRKNEDKLNHHQ |
| | RIGLKYFGDFEKRIPREEMLQMQDIVLNEVKKVDSEYIATVCGSF |
| | RRGAESSGDMDVLLTHPSFTSESTKQPKLLHQVVEQLQKVHFITD |
| | TLSKGETKFMGVCQLPSKNDEKEYPHRRIDIRLIPKDQYYCGVLY |
| | FTGSDIFNKNMRAHALEKGFTINEYTIRPLGVTGVAGEPLPVDSE |
| | KDIFDYIQWKYREPKDRSEGGGSGGGSATVKFKYKGEEKEVDIS |
| | KIKKVWRVGKMISFTYDEGGGKTGRGAVSEKDAPKELLQMLEK |
| | QKK (SEQ ID NO: 97) |
| Phi29 | MKHMPRKMYSCDFETTTKVEDCRVWAYGYMNIEDHSEYKIGNS |
| (D169A) | LDEFMAWVLKVQADLYFHNLKFDGAFIINWLERNGFKWSADGL |
| | PNTYNTIISRMGQWYMIDICLGYKGKRKIHTVIYDSLKKLPFPVK |
| | KIAKDFKLTVLKGDIDYHKERPVGYKITPEEYAYIKNAIQIIAEAL |
| | LIQFKQGLDRMTAGSDSLKGFKDIITTKKFKKVFPTLSLGLDKEV |
| | RYAYRGGFTWLNDRFKEKEIGEGMVFDVNSLYPAQMYSRLLPY |
| | GEPIVFEGKYVWDEDYPLHIQHIRCEFELKEGYIPTIQIKRSRFYKG |
| | NEYLKSSGGEIADLWLSNVDLELMKEHYDLYNVEYISGLKFKAT |
| | TGLFKDFIDKWTYIKTTSEGAIKQLAKLMLNSLYGKFASNPDVTG |
| | KVPYLKENGALGFRLGEEETKDPVYTPMGVFITAWARYTTITAA |
| | QACYDRIIYCDTDSIHLTGTEIPDVIKDIVDPKKLGYWAHESTFKR |
| | AKYLRQKTYIQDIYMKEVDGKLVEGSPDDYTDIKFSVKCAGMTD |
| | KIKKEVTFENFKVGFSRKMKPKPVQVPGGVVLVDDTFTIK (SEQ |
| | ID NO: 98) |
| Sequenase | MIVSDIEANALLESVTKFHCGVIYDYSTAEYVSYRPSDFGAYLDA |
| | LEAEVARGGLIVFHNGHKYDVPALTKLAKLQLNREFHLPRENCID |
| | TLVLSRLIHSNLKDTDMGLLRSGKLPGMLEEQGEEYVDGMEWW |
| | NFNEEMMDYNVQDVVVTKALLEKLLSDKHYFPPEIDFTDVGYTT |
| | FWSESLEAVDIEHRAAWLLAKQERNGFPFDTKAIEELYVELAARR |
| | SELLRKLTETFGSWYQPKGGTEMFCHPRTGKPLPKYPRIKTPKVG |
| | GIFKKPKNKAQREGREPCELDTREYVAGAPYTPVEHVVFNPSSRD |
| | HIQKKLQEAGWVPTKYTDKGAPVVDDEVLEGVRVDDPEKQAAI |
| | DLIKEYLMIQKRIGQSAEGDKAWLRYVAEDGKIHGSVNPNGAVT |
| | GRATHAFPNLAQIPGVRSPYGEQCRAAFGAEHHLDGITGKPWVQ |
| | AGIDASGLELRCLAHFMARFDNGEYAHEILNGDIHTKNQIAAELP |
| | TRDNAKTFIYGFLYGAGDEKIGQIVGAGKERGKELKKKFLENTPA |
| | IAALRESIQQTLVESSQWVAGEQQVKWKRRWIKGLDGRKVHVR |
| | SPHAALNTLLQSAGALICKLWIIKTEEMLVEKGLKHGWDGDFAY |
| | MAWVHDEIQVGCRTEEIAQVVIETAQEAMRWVGDHWNFRCLLD |
| | TEGKMGPNWAICH (SEQ ID NO: 99) |
| E. coli | MVISYDNYVTILDEETLKAWIAKLEKAPVFAFATATDSLDNISAN |
| dKlenow | LVGLSFAIEPGVAAYIPVAHDYLDAPDQISRERALELLKPLLEDEK |
| (D355A, | ALKVGQNLKYDRGILANYGIELRGIAFDTMLESYILNSVAGRHD |
| E357A, | MDSLAERWLKHKTITFEEIAGKGKNQLTFNQIALEEAGRYAAED |
| D705A, | ADVTLQLHLKMWPDLQKHKGPLNVFENIEMPLVPVLSRIERNGV |
| D882A) | KIDPKVLHNHSEELTLRLAELEKKAHEIAGEEFNLSSTKQLQTILF |
| | EKQGIKPLKKTPGGAPSTSEEVLEELALDYPLPKVILEYRGLAKLK |
| | STYTDKLPLMINPKTGRVHTSYHQAVTATGRLSSTDPNLQNIPVR |
| | NEEGRRIRQAFIAPEDYVIVSAAYSQIELRIMAHLSRDKGLLTAFA |
| | EGKDIHRATAAEVFGLPLETVTSEQRRSAKAINFGLIYGMSAFGL |
| | ARQLNIPRKEAQKYMDLYFERYPGVLEYMERTRAQAKEQGYVE |
| | TLDGRRLYLPDIKSSNGARRAAAERAAINAPMQGTAADIIKRAMI |
| | AVDAWLQAEQPRVRMIMQVHAELVFEVHKDDVDAVAKQIHQL |
| | MENCTRLDVPLLVEVGSGENWDQAH (SEQ ID NO: 100) |
| T4 DNA | MKEFYISIETVGNNIVERYIDENGKERTREVEYLPTMFRHCKEESKYKDIYGKNCAPQKF |
| polymerase | PSMKDARDWMKRMEDIGLEALGMNDFKLAYISDTYGSEIVYDRKFVRVANCDIEVTGDKF |
| (D189A, | PDPMKAEYEIDAITHYDSIDDRFYVFDLLNSMYGSVSKWDAKLAAKLDCEGGDEVPQEIL |
| E191A, | DRVIYMPFANARDMLMEYINLWEQKRPAIFTGWNIEGFDVPYIMNRVKMILGERSMKRFS |
| L412M) | PIGRVKSKLIQNMYGSKEIYSIDGVSILDYLDLYKKFAFTNLPSFSLESVAQHETKKGKL |
| | PYDGPINKLRETNHQRYISYNIIDVESVQAIDKIRGFIDLVLSMSYYAKMPFSGVMSPIK |
| | TWDAIIFNSLKGEHKVIPQQGSHVKQSFPGAFVFEPKPIARRYIMSFDLTSMYPSIIRQV |
| | NISPETIRGQFKVHPIHEYIAGTAPKPSDEYSCSPNGWMYDKHQEGIIPKEIAKVFFQRK |
| | DWKKKMFAEEMNAEAIKKIIMKGAGSCSTKPEVERYVKFSDDFLNELSNYTESVLNSLIE |
| | ECEKAATLANTNQLNRKILINSLYGALGNIHFRYYDLRNATAITIFGQVGIQWIARKINE |
| | YLNKVCGTNDEDFIAAGDTDSVYVCVDKVIEKVGLDRFKEQNDLVEFMNQFGKKKMEPMI |
| | DVAYRELCDYMNNREHLMHMDREAISCPPLGSKGVGGFWKAKKRYALNVYDMEDKRFAEP |
| | HLKIMGMETQQSSTPKAVQEALEESIRRILQEGEESVQEYYKNFEKEYRQLDYKVIAEVK |
| | TANDIAKYDDKGWPGFKCPFHIRGVLTYRRAVSGLGVAPILDGNKVMVLPLREGNPFGDK |
| | CIAWPSGTELPKEIRSDVLSWIDHSTLFQKSFVKPLAGMCESAGMDYEEKASLDFLFG |
| | (SEQ ID NO: 101) |
| TaqStoffel | SPKALEEAPWPPPEGAFVGFVLSRKEPMWADLLALAAARGGRVHRA |
| (E507K/ | PEPYKALRDLKEARGLLAKDLSVLALREGLGLPPGDDPMLLAYLLDPS |
| S543N/ | NTTPEGVARRYGGEWTEEAGERAALSERLFANLWGRLEGEERLLWL |
| E742R/ | YREVERPLSAVLAHMEATGVRLDVAYLRALSLEVAEEIARLEAEVFRL |
| A743K + | AGHPFNLNSRDQLERVLFDELGLPAIGKTKKTGKRSTSAAVLEALREA |
| P685R/ | HPIVEKILQYRELTKLKNTYIDPLPDLIHPRTGRLHTRFNQTATATGRLS |
| Y686R/ | SSDPNLQNIPVRTPLGQRIRRAFIAEEGWLLVALDYSQIELRVLAHLSG |
| E687R) | DENLIRVFQEGRDIHTETASWMFGVPREAVDPLMRRAAKTINFGVLY |
| | GMSAHRLSQELAIRRREAQAFIERYFQSFPKVRAWIEKTLEEGRRRG |
| | YVETLFGRRRYVPDLEARVKSVRRKAERMAFNMPVQGTAADLMKLA |
| | MVKLFPRLEEMGARMLLQVHDELVLEAPKERAEAVARLAKEVMEGV |
| | YPLAVPLEVEVGIGEDWLSAKE (SEQ ID NO: 102) |
| TaqStoffel | SPKALEEAPWPPPEGAFVGFVLSRKEPMWADLLALAAARGGRVH |
| (E507K/ | RAPEPYKALRDLKEARGLLAKDLSVLALREGLGLPPGDDPMLLAYL |
| S543N/ | LDPSNTTPEGVARRYGGEWTEEAGERAALSERLFANLWGRLEGE |
| E742R/ | ERLLWLYREVERPLSAVLAHMEATGVRLDVAYLRALSLEVAEEIARL |
| A743K + | EAEVFRLAGHPFNLNSRDQLERVLFDELGLPAIGKTKKTGKRSTSA |
| P685R/ | AVLEALREAHPIVEKILQYRELTKLKNTYIDPLPDLIHPRTGRLHTRF |
| Y686R/ | NQTATATGRLSSSDPNLQNIPVRTPLGQRIRRAFIAEEGWLLVALDY |
| E687R + | SQIELRVLAHLSGDENLIRVFQEGRDIHTETASWMFGVPREAVDPL |
| H676Y) | MRRAAKTINFGVLYGMSAYRLSQELAIRRREAQAFIERYFQSFPKV |
| | RAWIEKTLEEGRRRGYVETLFGRRRYVPDLEARVKSVRRKAERMA |
| | FNMPVQGTAADLMKLAMVKLFPRLEEMGARMLLQVHDELVLEAPK |
| | ERAEAVARLAKEVMEGVYPLAVPLEVEVGIGEDWLSAKE (SEQ ID |
| | NO: 103) |
| TaqStoffel | SPKALEEAPWPPPEGAFVGFVLSRKEPMWADLLALAAARGGRVH |
| (E507K/ | RAPEPYKALRDLKEARGLLAKDLSVLALREGLGLPPGDDPMLLAYL |
| S543N/ | LDPSNTTPEGVARRYGGEWTEEAGERAALSERLFANLWGRLEGE |
| E742R/ | ERLLWLYREVERPLSAVLAHMEATGVRLDVAYLRALSLEVAEEIARL |
| A743K + | EAEVFRLAGHPFNLNSRDQLERVLFDELGLPAIGKTKKTGKRSTSA |
| P685R/ | AVLEALREAHPIVEKILQYRELTKLKNTYIDPLPDLIHPRTGRLHTRF |
| Y686R/ | NQTATATGRLSSSDPNLQNIPVRTPLGQRIRRAFIAEEGWLLVALDY |
| E687R + 9 aa | SQIELRVLAHLSGDENLIRVFQEGRDIHTETASWMFGVPREAVDPL |
| ins) | MRRAAKTINFGVLYGMSAHRLSQELAIRRREAQAFIERYFQSFPKV |
| | RAWIEKTLEEGRRRGYVETLFGRRRYVPDLEARGPGQAPRRLVKS |
| | VRRKAERMAFNMPVQGTAADLMKLAMVKLFPRLEEMGARMLLQV |
| | HDELVLEAPKERAEAVARLAKEVMEGVYPLAVPLEVEVGIGEDWLS |
| | AK (SEQ ID NO: 104) |
| TaqStoffel | SPKALEEAPWPPPEGAFVGFVLSRKEPMWADLLALAAARGGRVH |
| (E507K/ | RAPEPYKALRDLKEARGLLAKDLSVLALREGLGLPPGDDPMLLAYL |
| S543N/ | LDPSNTTPEGVARRYGGEWTEEAGERAALSERLFANLWGRLEGE |
| E742R/ | ERLLWLYREVERPLSAVLAHMEATGVRLDVAYLRALSLEVAEEIARL |
| A743K + | EAEVFRLAGHPFNLNSRDQLERVLFDELGLPAIGKTKKTGKRSTSA |
| P685R/ | AVLEALREAHPIVEKILQYRELTKLKNTYIDPLPDLIHPRTGRLHTRF |
| Y686R/ | NQTATATGRLSSSDPNLQNIPVRTPLGQRIRRAFIAEEGWLLVALDY |
| E687R + | SQIELRVLAHLSGDENLIRVFQEGRDIHTETASWMFGVPREAVDPL |
| H676Y + | MRRAAKTINFGVLYGMSAYRLSQELAIRRREAQAFIERYFQSFPKV |
| 9 aa ins) | RAWIEKTLEEGRRRGYVETLFGRRRYVPDLEARGPGQAPRRLVKS |
| | VRRKAERMAFNMPVQGTAADLMKLAMVKLFPRLEEMGARMLLQV |
| | HDELVLEAPKERAEAVARLAKEVMEGVYPLAVPLEVEVGIGEDWLS |
| | A (SEQ ID NO: 105) |
| TaqStoffel | SPKALEEAPWPPPEGAFVGFVLSRKEPMWADLLALAAARGGRVH |
| (E507K/ | RAPEPYKALRDLKEARGLLAKDLSVLALREGLGLPPGDDPMLLAYL |
| S543N/ | LDPSNTTPEGVARRYGGEWTEEAGERAALSERLFANLWGRLEGE |
| E742R/ | ERLLWLYREVERPLSAVLAHMEATGVRLDVAYLRALSLEVAEEIARL |
| A743K + | EAEVFRLAGHPFNLNSRDQLERVLFDELGLPAIGKTKKTGKRSTSA |
| 9 aa ins) | AVLEALREAHPIVEKILQYRELTKLKNTYIDPLPDLIHPRTGRLHTRF |
| | NQTATATGRLSSSDPNLQNIPVRTPLGQRIRRAFIAEEGWLLVALDY |
| | SQIELRVLAHLSGDENLIRVFQEGRDIHTETASWMFGVPREAVDPL |
| | MRRAAKTINFGVLYGMSAHRLSQELAIPYEEAQAFIERYFQSFPKV |
| | RAWIEKTLEEGRRRGYVETLFGRRRYVPDLEARGPGQAPRRLVKS |
| | VRRKAERMAFNMPVQGTAADLMKLAMVKLFPRLEEMGARMLLQV |
| | HDELVLEAPKERAEAVARLAKEVMEGVYPLAVPLEVEVGIGEDWLS |
| | AKE (SEQ ID NO: 106) |
| EcKlenow_ | VISYDNYVTILDEETLKAWIAKLEKAPVFAFATATDSLDNISANLVGL |
| (A837R/ | SFAIEPGVAAYIPVAHDYLDAPDQISRERALELLKPLLEDEKALKVG |
| A838K + | QNLKYDRGILANYGIELRGIAFDTMLESYILNSVAGRHDMDSLAER |
| P780R) | WLKHKTITFEEIAGKGKNQLTFNQIALEEAGRYAAEDADVTLQLHLK |
| | MWPDLQKHKGPLNVFENIEMPLVPVLSRIERNGVKIDPKVLHNHSE |
| | ELTLRLAELEKKAHEIAGEEFNLSSTKQLQTILFEKQGIKPLKKTPGG |
| | APSTSEEVLEELALDYPLPKVILEYRGLAKLKSTYTDKLPLMINPKTG |
| | RVHTSYHQAVTATGRLSSTDPNLQNIPVRNEEGRRIRQAFIAPEDY |
| | VIVSADYSQIELRIMAHLSRDKGLLTAFAEGKDIHRATAAEVFGLPLE |
| | TVTSEQRRSAKAINFGLIYGMSAFGLARQLNIRRKEAQKYMDLYFE |
| | RYPGVLEYMERTRAQAKEQGYVETLDGRRLYLPDIKSSNGARRRK |
| | AERAAINAPMQGTAADIIKRAMIAVDAWLQAEQPRVRMIMQVHDEL |
| | VFEVHKDDVDAVAKQIHQLMENCTRLDVPLLVEVGSGENWDQAH |
| | (SEQ ID NO: 107) |
| EcKlenow | VISYDNYVTILDEETLKAWIAKLEKAPVFAFATATDSLDNISANLVGL |
| (A837R/ | SFAIEPGVAAYIPVAHDYLDAPDQISRERALELLKPLLEDEKALKVG |
| A838K + | QNLKYDRGILANYGIELRGIAFDTMLESYILNSVAGRHDMDSLAER |
| P780R + | WLKHKTITFEEIAGKGKNQLTFNQIALEEAGRYAAEDADVTLQLHLK |
| 9 aa ins) | MWPDLQKHKGPLNVFENIEMPLVPVLSRIERNGVKIDPKVLHNHSE |
| | ELTLRLAELEKKAHEIAGEEFNLSSTKQLQTILFEKQGIKPLKKTPGG |
| | APSTSEEVLEELALDYPLPKVILEYRGLAKLKSTYTDKLPLMINPKTG |
| | RVHTSYHQAVTATGRLSSTDPNLQNIPVRNEEGRRIRQAFIAPEDY |
| | VIVSADYSQIELRIMAHLSRDKGLLTAFAEGKDIHRATAAEVFGLPLE |
| | TVTSEQRRSAKAINFGLIYGMSAFGLARQLNIRRKEAQKYMDLYFE |
| | RYPGVLEYMERTRAQAKEQGYVETLDGRRLYLPDIKSSGPGQAPR |
| | RLNGARRRKAERAAINAPMQGTAADIIKRAMIAVDAWLQAEQPRVR |
| | MIMQVHDELVFEVHKDDVDAVAKQIHQLMENCTRLDVPLLVEVGS |
| | GENWDQAH (SEQ ID NO: 108) |
| EcKlenow_ | VISYDNYVTILDEETLKAWIAKLEKAPVFAFATATDSLDNISANLVGL |
| (A837R/ | SFAIEPGVAAYIPVAHDYLDAPDQISRERALELLKPLLEDEKALKVG |
| A838K + | QNLKYDRGILANYGIELRGIAFDTMLESYILNSVAGRHDMDSLAER |
| 9 aa ins) | WLKHKTITFEEIAGKGKNQLTFNQIALEEAGRYAAEDADVTLQLHLK |
| | MWPDLQKHKGPLNVFENIEMPLVPVLSRIERNGVKIDPKVLHNHSE |
| | ELTLRLAELEKKAHEIAGEEFNLSSTKQLQTILFEKQGIKPLKKTPGG |
| | APSTSEEVLEELALDYPLPKVILEYRGLAKLKSTYTDKLPLMINPKTG |
| | RVHTSYHQAVTATGRLSSTDPNLQNIPVRNEEGRRIRQAFIAPEDY |
| | VIVSADYSQIELRIMAHLSRDKGLLTAFAEGKDIHRATAAEVFGLPLE |
| | TVTSEQRRSAKAINFGLIYGMSAFGLARQLNIPRKEAQKYMDLYFE |
| | RYPGVLEYMERTRAQAKEQGYVETLDGRRLYLPDIKSSGPGQAPR |
| | RLNGARRRKAERAAINAPMQGTAADIIKRAMIAVDAWLQAEQPRVR |
| | MIMQVHDELVFEVHKDDVDAVAKQIHQLMENCTRLDVPLLVEVGS |
| | GENWDQAH (SEQ ID NO: 109) |
| EcKlenow_ | VISYDNYVTILDEETLKAWIAKLEKAPVFAFATATDSLDNISANLVGL |
| (A837R/ | SFAIEPGVAAYIPVAHDYLDAPDQISRERALELLKPLLEDEKALKVG |
| A838K/ | QNLKYDRGILANYGIELRGIAFDTMLESYILNSVAGRHDMDSLAER |
| P603K/ | WLKHKTITFEEIAGKGKNQLTFNQIALEEAGRYAAEDADVTLQLHLK |
| S638N + | MWPDLQKHKGPLNVFENIEMPLVPVLSRIERNGVKIDPKVLHNHSE |
| P780R) | ELTLRLAELEKKAHEIAGEEFNLSSTKQLQTILFEKQGIKPLKKTKGG |
| | APSTSEEVLEELALDYPLPKVILEYRGLAKLKNTYTDKLPLMINPKTG |
| | RVHTSYHQAVTATGRLSSTDPNLQNIPVRNEEGRRIRQAFIAPEDY |
| | VIVSADYSQIELRIMAHLSRDKGLLTAFAEGKDIHRATAAEVFGLPLE |
| | TVTSEQRRSAKAINFGLIYGMSAFGLARQLNIRRKEAQKYMDLYFE |
| | RYPGVLEYMERTRAQAKEQGYVETLDGRRLYLPDIKSSNGARRRK |
| | AERAAINAPMQGTAADIIKRAMIAVDAWLQAEQPRVRMIMQVHDEL |
| | VFEVHKDDVDAVAKQIHQLMENCTRLDVPLLVEVGSGENWDQAH |
| | (SEQ ID NO: 110) |
| EcKlenow_ | VISYDNYVTILDEETLKAWIAKLEKAPVFAFATATDSLDNISANLVGL |
| (A837R/ | SFAIEPGVAAYIPVAHDYLDAPDQISRERALELLKPLLEDEKALKVG |
| A838K/ | QNLKYDRGILANYGIELRGIAFDTMLESYILNSVAGRHDMDSLAER |
| P603K/ | WLKHKTITFEEIAGKGKNQLTFNQIALEEAGRYAAEDADVTLQLHLK |
| S638N + | MWPDLQKHKGPLNVFENIEMPLVPVLSRIERNGVKIDPKVLHNHSE |
| P780R + | ELTLRLAELEKKAHEIAGEEFNLSSTKQLQTILFEKQGIKPLKKTKGG |
| 9 aa ins) | APSTSEEVLEELALDYPLPKVILEYRGLAKLKNTYTDKLPLMINPKTG |
| | RVHTSYHQAVTATGRLSSTDPNLQNIPVRNEEGRRIRQAFIAPEDY |
| | VIVSADYSQIELRIMAHLSRDKGLLTAFAEGKDIHRATAAEVFGLPLE |
| | TVTSEQRRSAKAINFGLIYGMSAFGLARQLNIRRKEAQKYMDLYFE |
| | RYPGVLEYMERTRAQAKEQGYVETLDGRRLYLPDIKSSGPGQAPR |
| | RLNGARRRKAERAAINAPMQGTAADIIKRAMIAVDAWLQAEQPRVR |
| | MIMQVHDELVFEVHKDDVDAVAKQIHQLMENCTRLDVPLLVEVGS |
| | GENWDQAH (SEQ ID NO: 111) |
| EcKlenow_ | VISYDNYVTILDEETLKAWIAKLEKAPVFAFATATDSLDNISANLVGL |
| (A837R/ | SFAIEPGVAAYIPVAHDYLDAPDQISRERALELLKPLLEDEKALKVG |
| A838K/ | QNLKYDRGILANYGIELRGIAFDTMLESYILNSVAGRHDMDSLAER |
| P603K/ | WLKHKTITFEEIAGKGKNQLTFNQIALEEAGRYAAEDADVTLQLHLK |
| S638N + 9 aa | MWPDLQKHKGPLNVFENIEMPLVPVLSRIERNGVKIDPKVLHNHSE |
| ins) | ELTLRLAELEKKAHEIAGEEFNLSSTKQLQTILFEKQGIKPLKKTKGG |
| | APSTSEEVLEELALDYPLPKVILEYRGLAKLKNTYTDKLPLMINPKTG |
| | RVHTSYHQAVTATGRLSSTDPNLQNIPVRNEEGRRIRQAFIAPEDY |
| | VIVSADYSQIELRIMAHLSRDKGLLTAFAEGKDIHRATAAEVFGLPLE |
| | TVTSEQRRSAKAINFGLIYGMSAFGLARQLNIPRKEAQKYMDLYFE |
| | RYPGVLEYMERTRAQAKEQGYVETLDGRRLYLPDIKSSGPGQAPR |
| | RLNGARRRKAERAAINAPMQGTAADIIKRAMIAVDAWLQAEQPRVR |
| | MIMQVHDELVFEVHKDDVDAVAKQIHQLMENCTRLDVPLLVEVGS |
| | GENWDQAH (SEQ ID NO: 112) |
| MMLV-RT | TLNIEDEYRLHETSKEPDVSLGSTWLSDFPQAWAETGGMGLAVRQ |
| | APLIIPLKATSTPVSIKQYPMSQEARLGIKPHIQRLLDQGILVPCQSP |
| | WNTPLLPVKKPGTNDYRPVQDLREVNKRVEDIHPTVPNPYNLLSGL |
| | PPSHQWYTVLDLKDAFFCLRLHPTSQPLFAFEWRDPEMGISGQLT |
| | WTRLPQGFKNSPTLFDEALHRDLADFRIQHPDLILLQYVDDLLLAAT |
| | SELDCQQGTRALLQTLGNLGYRASAKKAQICQKQVKYLGYLLKEG |
| | QRWLTEARKETVMGQPTPKTPRQLREFLGTAGFCRLWIPGFAEMA |
| | APLYPLTKTGTLFNWGPDQQKAYQEIKQALLTAPALGLPDLTKPFEL |
| | FVDEKQGYAKGVLTQKLGPWRRPVAYLSKKLDPVAAGWPPCLRM |
| | VAAIAVLTKDAGKLTMGQPLVILAPHAVEALVKQPPDRWLSNARMT |
| | HYQALLLDTDRVQFGPVVALNPATLLPLPEEGLQHNCLDILAEAHG |
| | TRPDLTDQPLPDADHTWYTDGSSLLQEGQRKAGAAVTTETEVIWA |
| | KALPAGTSAQRAELIALTQALKMAEGKKLNVYTDSRYAFATAHIHGE |
| | IYRRRGLLTSEGKEIKNKDEILALLKALFLPKRLSIIHCPGHQKGHSA |
| | EARGNRMADQAARKAAITETPDTSTLLIENSSP (SEQ ID NO: 113) |
| MMTV-RT | VFTLWGRDIMKDIKVRLMTDSPDDSQDLMIGAIESNLFADQISWKSDQ |
| | PVWLNQWPLKQEKLQALQQLVTEQLQLGHLEESNSPWNTPVFVIKKK |
| | SGKWRLLQDLRAVNATMHDMGALQPGLPSPVAVPKGWEIIIIDLQDCF |
| | FNIKLHPEDCKRFAFSVPSPNFKRPYQRFQWKVLPQGMKNSPTLCQK |
| | FVDKAILTVRDKYQDSYIVHYMDDILLAHPSRSIVDEILTSMIQALNKHGL |
| | VVSTEKIQKYDNLKYLGTHIQGDSVSYQKLQIRTDKLRTLNDFQKLLGNI |
| | NWIRPFLKLTTGELKPLFEILNGDSNPISTRKLTPEACKALQLMNERLST |
| | ARVKRLDLSQPWSLCILKTEYTPTACLWQDGVVEWIHLPHISPKVITPY |
| | DIFCTQLIIKGRHRSKELFSKDPDYIVVPYTKVQFDLLLQEKEDWPISLL |
| | GFLGEVHFHLPKDPLLTFTLQTAIIFPHMTSTTPLEKGIVIFTDGSANGR |
| | SVTYIQGREPIIKENTQNTAQQAEIVAVITAFEEVSQPFNLYTDSKYVTG |
| | LFPEIETATLSPRTKIYTELKHLQRLIHKRQEKFYIGHIRGHTGLPGPLAQ |
| | GNAYADSLTRILT (SEQ ID NO: 114) |
| ASLV-RT | TVALHLAIPLKWKPDHTPVWIDQWPLPEGKLVALTQLVEKELQLGHIEP |
| | SLSCWNTPVFVIRKASGSYRLLHDLRAVNAKLVPFGAVQQGAPVLSAL |
| | PRGWPLMVLDLKDCFFSIPLAEQDREAFAFTLPSVNNQAPARRFQWK |
| | VLPQGMTCSPTICQLVVGQVLEPLRLKHPSLRMLHYMDDLLLAASSHD |
| | GLEAAGEEVISTLERAGFTISPDKIQREPGVQYLGYKLGSTYVAPVGLV |
| | AEPRIATLWDVQKLVGSLQWLRPALGIPPRLMGPFYEQLRGSDPNEA |
| | REWNLDMKMAWREIVQLSTTAALERWDPALPLEGAVARCEQGAIGVL |
| | GQGLSTHPRPCLWLFSTQPTKAFTAWLEVLTLLITKLRASAVRTFGKE |
| | VDILLLPACFREDLPLPEGILLALKGFAGKIRSSDTPSIFDIARPLHVSLK |
| | VRVTDHPVPGPTVFTDASSSTHKGVVVWREGPRWEIKEIADSGASVQ |
| | QLEARAVAMALLLWPTTPTNVVTDSAFVAKMLLKMGQEGVPSTAAAFI |
| | LEDALSQRSAMAAVLHVRSHSEVPGFFTEGNDVADSQATFQAY (SEQ |
| | ID NO: 115) |
| PERV-RT | TLQLDDEYRLYSPQVKPDQDIQSWLEQFPQAWAETAGMGLAKQVPP |
| | QVIQLKASATPVSVRQYPLSREAREGIWPHVQRLIQQGILVPVQSPWN |
| | TPLLPVRKPGTNDYRPVQDLREVNKRVQDIHPTVPNPYNLLSALPPER |
| | NWYTVLDLKDAFFCLRLHPTSQPLFAFEWRDPGTGRTGQLTWTRLPQ |
| | GFKNSPTIFDEALHRDLANFRIQHPQVTLLQYVDDLLLAGATKQDCLEG |
| | TKALLLELSDLGYRASAKKAQICRREVTYLGYSLRGGQRWLTEARKKT |
| | VVQIPAPTTAKQVREFLGTAGFCRLWIPGFATLAAPLYPLTKEKGEFS |
| | WAPEHQKAFDAIKKALLSAPALALPDVTKPFTLYVDERKGVARGVLTQ |
| | TLGPWRRPVAYLSKKLDPVASGWPVCLKAIAAVAILVKDADKLTLGQNI |
| | TVIAPHALENIVRQPPDRWMTNARMTHYQSLLLTERVTFAPPAALNPA |
| | TLLPEETDEPVTHDCHQLLIEETGVRKDLTDIPLTGEVLTWFTDGSSYV |
| | VEGKRMAGAAVVDGTHTIWASSLPEGTSAQKAELMALTQALRLAEGK |
| | SINIYTDSRYAFATAHVHGAIYKQRGLLTSAGREIKNKEEILSLLEALHLP |
| | KRLAIIHCPGHQKAKDLISRGNQMADRVAKQAAQAVNLLPI (SEQ ID |
| | NO: 116) |
| AVIRE-RT | APLEEEYRLFLEAPIQNVTLLEQWKREIPKVWAEINPPGLASTQAPIHV |
| | QLLSTALPVRVRQYPITLEAKRSLRETIRKFRAAGILRPVHSPWNTPLLP |
| | VRKSGTSEYRMVQDLREVNKRVETIHPTVPNPYTLLSLLPPDRIWYSV |
| | LDLKDAFFCIPLAPESQLIFAFEWADAEEGESGQLTWTRLPQGFKNSP |
| | TLFDEALNRDLQGFRLDHPSVSLLQYVDDLLIAADTQAACLSATRDLLM |
| | TLAELGYRVSGKKAQLCQEEVTYLGFKIHKGSRSLSNSRTQAILQIPVP |
| | KTKRQVREFLGTIGYCRLWIPGFAELAQPLYAATRGGNDPLVWGEKE |
| | EEAFQSLKLALTQPPALALPSLDKPFQLFVEETSGAAKGVLTQALGPW |
| | KRPVAYLSKRLDPVAAGWPRCLRAIAAAALLTREASKLTFGQDIEITSS |
| | HNLESLLRSPPDKWLTNARITQYQVLLLDPPRVRFKQTAALNPATLLPE |
| | TDDTLPIHHCLDTLDSLTSTRPDLTDQPLAQAEATLFTDGSSYIRDGKR |
| | YAGAAVVTLDSVIWAEPLPIGTSAQKAELIALTKALEWSKDKSVNIYTDS |
| | RYAFATLHVHGMIYRERGLLTAGGKAIKNAPEILALLTAVWLPKRVAVM |
| | HCKGHQKDDAPTSTGNRRADEVAREVAIRPLSTQATISDAPDMPDTET |
| | PQYSNVEEALG (SEQ ID NO: 117) |
| BAEMV- | VSLQDEHRLFDIPVTTSLPDVWLQDFPQAWAETGGLGRAKCQAPIIIDL |
| RT | KPTAVPVSIKQYPMSLEAHMGIRQHIIKFLELGVLRPCRSPWNTPLLPV |
| | KKPGTQDYRPVQDLREINKRTVDIHPTVPNPYNLLSTLKPDYSWYTVL |
| | DLKDAFFCLPLAPQSQELFAFEWKDPERGISGQLTWTRLPQGFKNSP |
| | TLFDEALHRDLTDFRTQHPEVTLLQYVDDLLLAAPTKKACTQGTRHLL |
| | QELGEKGYRASAKKAQICQTKVTYLGYILSEGKRWLTPGRIETVARIPP |
| | PRNPREVREFLGTAGFCRLWIPGFAELAAPLYALTKESTPFTWQTEHQ |
| | LAFEALKKALLSAPALGLPDTSKPFTLFLDERQGIAKGVLTQKLGPWKR |
| | PVAYLSKKLDPVAAGWPPCLRIMAATAMLVKDSAKLTLGQPLTVITPHT |
| | LEAIVRQPPDRWITNARLTHYQALLLDTDRVQFGPPVTLNPATLLPVPE |
| | NQPSPHDCRQVLAETHGTREDLKDQELPDADHTWYTDGSSYLDSGT |
| | RRAGAAVVDGHNTIWAQSLPPGTSAQKAELIALTKALELSKGKKANIYT |
| | DSRYAFATAHTHGSIYERRGLLTSEGKEIKNKAEIIALLKALFLPQEVAII |
| | HCPGHQKGQDPVAVGNRQADRVARQAAMAEVLTLATEPDNTSHITIE |
| | HTYTSEDQEEA (SEQ ID NO: 118) |

Exemplary Recombinase sequences

| name | aa sequence |
|---|---|
| BxbI | MRALVVIRLSRVTDATTSPERQLESCQQLCAQRGWDVVGVAEDL |
| | DVSGAVDPFDRKRRPNLARWLAFEEQPFDVIVAYRVDRLTRSIRH |
| | LQQLVHWAEDHKKLVVSATEAHFDTTTPFAAVVIALMGTVAQM |
| | ELEAIKERNRSAAHFNIRAGKYRGSLPPWGYLPTRVDGEWRLVP |
| | DPVQRERILEVYHRVVDNHEPLHLVAHDLNRRGVLSPKDYFAQL |
| | QGREPQGREWSATALKRSMISEAMLGYATLNGKTVRDDDGAPL |
| | VRAEPILTREQLEALRAELVKTSRAKPAVSTPSLLLRVLFCAVCGE |
| | PAYKFAGGGRKHPRYRCRSMGFPKHCGNGTVAMAEWDAFCEE |
| | QVLDLLGDAERLEKVWVAGSDSAVELAEVNAELVDLTSLIGSPA |
| | YRAGSPQREALDARIAALAARQEELEGLEARPSGWEWRETGQRF |
| | GDWWREQDTAAKNTWLRSMNVRLTFDVRGGLTRTIDFGDLQEY |
| | EQHLRLGSVVERLHTGMS (SEQ ID NO: 119) |
| Pa01 | MGPSAFSYVRFSSGKQAKGSSEHRQRAMLGQWLEQHPSFTLSDL |
| | RFEDLGRSGFSGEHLDHGLGQLLAAIDSGAIKSGDVILVEAVDRIG |
| | RLEPLEMLPLFSRIVKAGVSVITLEDGHVYDRSSVNETSLFLLVAK |
| | IQQAHEYSNRLSRRINASYTARREKAKAGLGIKRETPVWLTTDGQ |
| | LVPHVAPHIAQAFQDYADGLGERRICRKLRESGLEEFSKTNATTV |
| | RRWLKNRTAIGYWNDIPDVYPHVVDPALFYQVQQRLDAPKVDR |
| | AKPSAHYLTGLVKCAVCGRNYNYKQRKHTDPAMLCTSRARLAG |
| | EGCSNSKTYPVIVLDQVRKLTSLPFLQHAMESASSQADPSSQRLA |
| | VIDGEIGELSRKISEATKALLVLGFTPEIQESLEQLKTAREALEEER |
| | ATLLLPQAEKLTTAQLEAFSNGLLDDEPMKLNHVLQTAGYSMVV |
| | HPDGSIDVDGKRFVYEGASRKEKVYKLRLIGEDKQWSLPILTPQM |
| | ATYKSLFMAAVRLPGDPSEEELRRFEEAKHSER (SEQ ID NO: 120) |
| NLS- | MPKKKRKVYPYDVPDYAGSYRPESLDVCIYLRKSRKDVEEERRA |
| BceINT | IEEGSSYNALERHRKRLFAIAKAENHNIIDIFEEVASGESIQERPQM |
| | QQLLRKLEGNEIDGVLVIDLDRLGRGDMLDAGMIDRAFRYSSTKI |
| | ITPTDVYDPDDESWELVFGIKSLISRQELKSITKRLQNGRIDSVKEG |
| | KHIGKKPPYGYLKDENLRLYPDPEKAWIVKKIFELMCDGKGRQM |
| | IAAELDRLGIDPPVTKRGAWDSSTITSIIKNEVYTGVIVWGKFKHK |
| | KRNGKYTRHKNPQEKWIMYENAHEPIISKELFDAANEAHSSRHK |
| | PAVITSKKLTNPLAGILKCKLCGYTMLIQTRKDRPHNYLRCNNPA |
| | CKGKQKQSVFNLVEEKLLYSLQQIVDEYQAQKVEEVEIDDSKLIS |
| | FKEKAIISKEKELKELQAQKGNLHDLLEQGIYTVEIFLERQKNLVE |
| | RITSIENDIEVLQKEIETEQIKEHNKTEFIPALKTVIESYHKTTNIEL |
| | KNQLLKTILSTVTYYRHPDWKTNEFEIQVYFKIS (SEQ ID NO: |
| | 121) |

Exemplary Full LCE construct amino acid sequences

| name | aa sequence |
|---|---|
| PCV2- | MSPSKKNGRSGPQPHKRWVFTLNNPSEDERKKIRDLPISLFDYFIVGEEGNEE |
| XTEN- | GRTPHLQGFANFVKKQTFNKVKWYLGARCHIEKAKGTDQQNKEYCSKEGN |
| nCas9 | LLMECGAPRSQGQRSGGSSGSETPGTSESATPESSGGSDKKYSIGLDIGTNSV |
| (H840A)- | GWAVITDEYKVPSKKFKVLGNTDRHSIKKNLIGALLFDSGETAEATRLKRTA |
| SGGS | RRRYTRRKNRICYLQEIFSNEMAKVDDSFFHRLEESFLVEEDKKHERHPIFGN |
| (SEQ ID | IVDEVAYHEKYPTIYHLRKKLVDSTDKADLRLIYLALAHMIKFRGHFLIEGDL |
| NO: | NPDNSDVDKLFIQLVQTYNQLFEENPINASGVDAKAILSARLSKSRRLENLIA |
| 573)_ | QLPGEKKNGLFGNLIALSLGLTPNFKSNFDLAEDAKLQLSKDTYDDDLDNLL |
| NLS_ | AQIGDQYADLFLAAKNLSDAILLSDILRVNTEITKAPLSASMIKRYDEHHQDL |
| SGGS | TLLKALVRQQLPEKYKEIFFDQSKNGYAGYIDGGASQEEFYKFIKPILEKMDG |
| (SEQ ID | TEELLVKLNREDLLRKQRTFDNGSIPHQIHLGELHAILRRQEDFYPFLKDNRE |
| NO: | KIEKILTFRIPYYVGPLARGNSRFAWMTRKSEETITPWNFEEVVDKGASAQSF |
| 573)- | IERMTNFDKNLPNEKVLPKHSLLYEYFTVYNELTKVKYVTEGMRKPAFLSGE |
| T3Lig- | QKKAIVDLLFKTNRKVTVKQLKEDYFKKIECFDSVEISGVEDRFNASLGTYH |
| NLS | DLLKIIKDKDFLDNEENEDILEDIVLTLTLFEDREMIEERLKTYAHLFDDKVM |
| | KQLKRRRYTGWGRLSRKLINGIRDKQSGKTILDFLKSDGFANRNFMQLIHDD |
| | SLTFKEDIQKAQVSGQGDSLHEHIANLAGSPAIKKGILQTVKVVDELVKVMG |
| | RHKPENIVIEMARENQTTQKGQKNSRERMKRIEEGIKELGSQILKEHPVENTQ |
| | LQNEKLYLYYLQNGRDMYVDQELDINRLSDYDVDAIVPQSFLKDDSIDNKV |
| | LTRSDKNRGKSDNVPSEEVVKKMKNYWRQLLNAKLITQRKFDNLTKAERG |
| | GLSELDKAGFIKRQLVETRQITKHVAQILDSRMNTKYDENDKLIREVKVITLK |
| | SKLVSDFRKDFQFYKVREINNYHHAHDAYLNAVVGTALIKKYPKLESEFVY |
| | GDYKVYDVRKMIAKSEQEIGKATAKYFFYSNIMNFFKTEITLANGEIRKRPLI |
| | ETNGETGEIVWDKGRDFATVRKVLSMPQVNIVKKTEVQTGGFSKESILPKRN |
| | SDKLIARKKDWDPKKYGGFDSPTVAYSVLVVAKVEKGKSKKLKSVKELLGI |
| | TIMERSSFEKNPIDFLEAKGYKEVKKDLIIKLPKYSLFELENGRKRMLASAGE |
| | LQKGNELALPSKYVNFLYLASHYEKLKGSPEDNEQKQLFVEQHKHYLDEIIE |
| | QISEFSKRVILADANLDKVLSAYNKHRDKPIREQAENIIHLFTLTNLGAPAAFK |
| | YFDTTIDRKRYTSTKEVLDATLIHQSITGLYETRIDLSQLGGDSGGSSGGSKRT |
| | ADGSEFESPKKKRKVSGGSSGGSNIFNTNPFKAVSFVESAVKKALETSGYLIA |
| | DCKYDGVRGNIVVDNVAEAAWLSRVSKFIPALEHLNGFDKRWQQLLNDDR |
| | CIFPDGFMLDGELMVKGVDFNTGSGLLRTKWVKRDNMGFHLTNVPTKLTPK |
| | GREVIDGKFEFHLDPKRLSVRLYAVMPIHIAESGEDYDVQNLLMPYHVEAM |
| | RSLLVEYFPEIEWLIAETYEVYDMDSLTELYEEKRAEGHEGLIVKDPQGIYKR |
| | GKKSGWWKLKPECEADGIIQGVNWGTEGLANEGKVIGFSVLLETGRLVDAN |
| | NISRALMDEFTSNVKAHGEDFYNGWACQVNYMEATPDGSLRHPSFEKFRGT |
| | EDNPQEKMSGGSKRTADGSEFESPKKKRKV (SEQ ID NO: 122) |
| PCV2- | MSPSKKNGRSGPQPHKRWVFTLNNPSEDERKKIRDLPISLFDYFIVGEEGNEE |
| XTEN- | GRTPHLQGFANFVKKQTFNKVKWYLGARCHIEKAKGTDQQNKEYCSKEGN |
| nCas9 | LLMECGAPRSQGQRSGGSSGSETPGTSESATPESSGGSDKKYSIGLDIGTNSV |
| (H840A)- | GWAVITDEYKVPSKKFKVLGNTDRHSIKKNLIGALLFDSGETAEATRLKRTA |
| SGGS | RRRYTRRKNRICYLQEIFSNEMAKVDDSFFHRLEESFLVEEDKKHERHPIFGN |
| (SEQ ID | IVDEVAYHEKYPTIYHLRKKLVDSTDKADLRLIYLALAHMIKFRGHFLIEGDL |
| NO: | NPDNSDVDKLFIQLVQTYNQLFEENPINASGVDAKAILSARLSKSRRLENLIA |
| 573)_ | QLPGEKKNGLFGNLIALSLGLTPNFKSNFDLAEDAKLQLSKDTYDDDLDNLL |
| NLS_ | AQIGDQYADLFLAAKNLSDAILLSDILRVNTEITKAPLSASMIKRYDEHHQDL |
| SGGS | TLLKALVRQQLPEKYKEIFFDQSKNGYAGYIDGGASQEEFYKFIKPILEKMDG |
| (SEQ ID | TEELLVKLNREDLLRKQRTFDNGSIPHQIHLGELHAILRRQEDFYPFLKDNRE |
| NO: | KIEKILTFRIPYYVGPLARGNSRFAWMTRKSEETITPWNFEEVVDKGASAQSF |
| 573)- | TERMTNFDKNLPNEKVLPKHSLLYEYFTVYNELTKVKYVTEGMRKPAFLSGE |
| T4Lig- | QKKAIVDLLFKTNRKVTVKQLKEDYFKKIECFDSVEISGVEDRFNASLGTYH |
| NLS | DLLKIIKDKDFLDNEENEDILEDIVLTLTLFEDREMIEERLKTYAHLFDDKVM |
| | KQLKRRRYTGWGRLSRKLINGIRDKQSGKTILDFLKSDGFANRNFMQLIHDD |
| | SLTFKEDIQKAQVSGQGDSLHEHIANLAGSPAIKKGILQTVKVVDELVKVMG |
| | RHKPENIVIEMARENQTTQKGQKNSRERMKRIEEGIKELGSQILKEHPVENTQ |
| | LQNEKLYLYYLQNGRDMYVDQELDINRLSDYDVDAIVPQSFLKDDSIDNKV |
| | LTRSDKNRGKSDNVPSEEVVKKMKNYWRQLLNAKLITQRKFDNLTKAERG |
| | GLSELDKAGFIKRQLVETRQITKHVAQILDSRMNTKYDENDKLIREVKVITLK |
| | SKLVSDFRKDFQFYKVREINNYHHAHDAYLNAVVGTALIKKYPKLESEFVY |
| | GDYKVYDVRKMIAKSEQEIGKATAKYFFYSNIMNFFKTEITLANGEIRKRPLI |
| | ETNGETGEIVWDKGRDFATVRKVLSMPQVNIVKKTEVQTGGFSKESILPKRN |
| | SDKLIARKKDWDPKKYGGFDSPTVAYSVLVVAKVEKGKSKKLKSVKELLGI |
| | TIMERSSFEKNPIDFLEAKGYKEVKKDLIIKLPKYSLFELENGRKRMLASAGE |
| | LQKGNELALPSKYVNFLYLASHYEKLKGSPEDNEQKQLFVEQHKHYLDEIIE |
| | QISEFSKRVILADANLDKVLSAYNKHRDKPIREQAENIIHLFTLTNLGAPAAFK |
| | YFDTTIDRKRYTSTKEVLDATLIHQSITGLYETRIDLSQLGGDSGGSSGGSKRT |
| | ADGSEFESPKKKRKVSGGSSGGSILKILNEIASIGSTKQKQAILEKNKDNELLK |
| | RVYRLTYSRGLQYYIKKWPKPGIATQSFGMLTLTDMLDFIEFTLATRKLTGN |
| | AAIEELTGYITDGKKDDVEVLRRVMMRDLECGASVSIANKVWPGLIPEQPQ |
| | MLASSYDEKGINKNIKFPAFAQLKADGARCFAEVRGDELDDVRLLSRAGNE |
| | YLGLDLLKEELIKMTAEARQIHPEGVLIDGELVYHEQVKKEPEGLDFLFDAY |
| | PENSKAKEFAEVAESRTASNGIANKSLKGTISEKEAQCMKFQVWDYVPLVEI |
| | YSLPAFRLKYDVRFSKLEQMTSGYDKVILIENQVVNNLDEAKVIYKKYIDQG |
| | LEGIILKNIDGLWENARSKNLYKFKEVIDVDLKIVGIYPHRKDPTKAGGFILES |
| | ECGKIKVNAGSGLKDKAGVKSHELDRTRIMENQNYYIGKILECECNGWLKS |
| | DGRTDYVKLFLPIAIRLREDKTKANTFEDVFGDFHEVTGLSGGSKRTADGSEF |
| | ESPKKKRKV (SEQ ID NO: 123) |
| PCV2- | MSPSKKNGRSGPQPHKRWVFTLNNPSEDERKKIRDLPISLFDYFIVGEEGNEE |
| XTEN- | GRTPHLQGFANFVKKQTFNKVKWYLGARCHIEKAKGTDQQNKEYCSKEGN |
| nCas9 | LLMECGAPRSQGQRSGGSSGSETPGTSESATPESSGGSDKKYSIGLDIGTNSV |
| (H840A)- | GWAVITDEYKVPSKKFKVLGNTDRHSIKKNLIGALLFDSGETAEATRLKRTA |
| SGGS | RRRYTRRKNRICYLQEIFSNEMAKVDDSFFHRLEESFLVEEDKKHERHPIFGN |
| (SEQ ID | IVDEVAYHEKYPTIYHLRKKLVDSTDKADLRLIYLALAHMIKFRGHFLIEGDL |
| NO: | NPDNSDVDKLFIQLVQTYNQLFEENPINASGVDAKAILSARLSKSRRLENLIA |
| 573)_ | QLPGEKKNGLFGNLIALSLGLTPNFKSNFDLAEDAKLQLSKDTYDDDLDNLL |
| NLS_ | AQIGDQYADLFLAAKNLSDAILLSDILRVNTEITKAPLSASMIKRYDEHHQDL |
| SGGS | TLLKALVRQQLPEKYKEIFFDQSKNGYAGYIDGGASQEEFYKFIKPILEKMDG |
| (SEQ ID | TEELLVKLNREDLLRKQRTFDNGSIPHQIHLGELHAILRRQEDFYPFLKDNRE |
| NO: | KIEKILTFRIPYYVGPLARGNSRFAWMTRKSEETITPWNFEEVVDKGASAQSF |
| 573)- | IERMTNFDKNLPNEKVLPKHSLLYEYFTVYNELTKVKYVTEGMRKPAFLSGE |
| T7Lig- | QKKAIVDLLFKTNRKVTVKQLKEDYFKKIECFDSVEISGVEDRFNASLGTYH |
| NLS | DLLKIIKDKDFLDNEENEDILEDIVLTLTLFEDREMIEERLKTYAHLFDDKVM |
| | KQLKRRRYTGWGRLSRKLINGIRDKQSGKTILDFLKSDGFANRNFMQLIHDD |
| | SLTFKEDIQKAQVSGQGDSLHEHIANLAGSPAIKKGILQTVKVVDELVKVMG |
| | RHKPENIVIEMARENQTTQKGQKNSRERMKRIEEGIKELGSQILKEHPVENTQ |
| | LQNEKLYLYYLQNGRDMYVDQELDINRLSDYDVDAIVPQSFLKDDSIDNKV |
| | LTRSDKNRGKSDNVPSEEVVKKMKNYWRQLLNAKLITQRKFDNLTKAERG |
| | GLSELDKAGFIKRQLVETRQITKHVAQILDSRMNTKYDENDKLIREVKVITLK |
| | SKLVSDFRKDFQFYKVREINNYHHAHDAYLNAVVGTALIKKYPKLESEFVY |
| | GDYKVYDVRKMIAKSEQEIGKATAKYFFYSNIMNFFKTEITLANGEIRKRPLI |
| | ETNGETGEIVWDKGRDFATVRKVLSMPQVNIVKKTEVQTGGFSKESILPKRN |
| | SDKLIARKKDWDPKKYGGFDSPTVAYSVLVVAKVEKGKSKKLKSVKELLGI |
| | TIMERSSFEKNPIDFLEAKGYKEVKKDLIIKLPKYSLFELENGRKRMLASAGE |
| | LQKGNELALPSKYVNFLYLASHYEKLKGSPEDNEQKQLFVEQHKHYLDEIIE |
| | QISEFSKRVILADANLDKVLSAYNKHRDKPIREQAENIIHLFTLTNLGAPAAFK |
| | YFDTTIDRKRYTSTKEVLDATLIHQSITGLYETRIDLSQLGGDSGGSSGGSKRT |
| | ADGSEFESPKKKRKVSGGSSGGSNIKTNPFKAVSFVESAIKKALDNAGYLIAE |
| | IKYDGVRGNICVDNTANSYWLSRVSKTIPALEHLNGFDVRWKRLLNDDRCF |
| | YKDGFMLDGELMVKGVDFNTGSGLLRTKWTDTKNQEFHEELFVEPIRKKDK |
| | VPFKLHTGHLHIKLYAILPLHIVESGEDCDVMTLLMQEHVKNMLPLLQEYFP |
| | EIEWQAAESYEVYDMVELQQLYEQKRAEGHEGLIVKDPMCIYKRGKKSGW |
| | WKMKPENEADGIIQGLVWGTKGLANEGKVIGFEVLLESGRLVNATNISRAL |
| | MDEFTETVKEATLSQWGFFSPYGIGDNDACTINPYDGWACQISYMEETPDGS |
| | LRHPSFVMFRGTEDNPQEKMSGGSKRTADGSEFESPKKKRKV (SEQ ID |
| | NO: 124) |
| PCV2- | MSPSKKNGRSGPQPHKRWVFTLNNPSEDERKKIRDLPISLFDYFIVGEEGNEE |
| XTEN- | GRTPHLQGFANFVKKQTFNKVKWYLGARCHIEKAKGTDQQNKEYCSKEGN |
| nCas9 | LLMECGAPRSQGQRSGGSSGSETPGTSESATPESSGGSDKKYSIGLDIGTNSV |
| (H840A)- | GWAVITDEYKVPSKKFKVLGNTDRHSIKKNLIGALLFDSGETAEATRLKRTA |
| SGGS | RRRYTRRKNRICYLQEIFSNEMAKVDDSFFHRLEESFLVEEDKKHERHPIFGN |
| (SEQ ID | IVDEVAYHEKYPTIYHLRKKLVDSTDKADLRLIYLALAHMIKFRGHFLIEGDL |
| NO: | NPDNSDVDKLFIQLVQTYNQLFEENPINASGVDAKAILSARLSKSRRLENLIA |
| 573)_ | QLPGEKKNGLFGNLIALSLGLTPNFKSNFDLAEDAKLQLSKDTYDDDLDNLL |
| NLS_ | AQIGDQYADLFLAAKNLSDAILLSDILRVNTEITKAPLSASMIKRYDEHHQDL |
| SGGS | TLLKALVRQQLPEKYKEIFFDQSKNGYAGYIDGGASQEEFYKFIKPILEKMDG |
| (SEQ ID | TEELLVKLNREDLLRKQRTFDNGSIPHQIHLGELHAILRRQEDFYPFLKDNRE |
| NO: | KIEKILTFRIPYYVGPLARGNSRFAWMTRKSEETITPWNFEEVVDKGASAQSF |
| 573)- | TERMTNFDKNLPNEKVLPKHSLLYEYFTVYNELTKVKYVTEGMRKPAFLSGE |
| ChlVLig- | QKKAIVDLLFKTNRKVTVKQLKEDYFKKIECFDSVEISGVEDRFNASLGTYH |
| NLS | DLLKIIKDKDFLDNEENEDILEDIVLTLTLFEDREMIEERLKTYAHLFDDKVM |
| | KQLKRRRYTGWGRLSRKLINGIRDKQSGKTILDFLKSDGFANRNFMQLIHDD |
| | SLTFKEDIQKAQVSGQGDSLHEHIANLAGSPAIKKGILQTVKVVDELVKVMG |
| | RHKPENIVIEMARENQTTQKGQKNSRERMKRIEEGIKELGSQILKEHPVENTQ |
| | LQNEKLYLYYLQNGRDMYVDQELDINRLSDYDVDAIVPQSFLKDDSIDNKV |
| | LTRSDKNRGKSDNVPSEEVVKKMKNYWRQLLNAKLITQRKFDNLTKAERG |
| | GLSELDKAGFIKRQLVETRQITKHVAQILDSRMNTKYDENDKLIREVKVITLK |
| | SKLVSDFRKDFQFYKVREINNYHHAHDAYLNAVVGTALIKKYPKLESEFVY |
| | GDYKVYDVRKMIAKSEQEIGKATAKYFFYSNIMNFFKTEITLANGEIRKRPLI |
| | ETNGETGEIVWDKGRDFATVRKVLSMPQVNIVKKTEVQTGGFSKESILPKRN |
| | SDKLIARKKDWDPKKYGGFDSPTVAYSVLVVAKVEKGKSKKLKSVKELLGI |
| | TIMERSSFEKNPIDFLEAKGYKEVKKDLIIKLPKYSLFELENGRKRMLASAGE |
| | LQKGNELALPSKYVNFLYLASHYEKLKGSPEDNEQKQLFVEQHKHYLDEIIE |
| | QISEFSKRVILADANLDKVLSAYNKHRDKPIREQAENIIHLFTLTNLGAPAAFK |
| | YFDTTIDRKRYTSTKEVLDATLIHQSITGLYETRIDLSQLGGDSGGSSGGSKRT |
| | ADGSEFESPKKKRKVSGGSSGGSAITKPLLAATLENIEDVQFPCLATPKIDGIR |
| | SVKQTQMLSRTFKPIRNSVMNRLLTELLPEGSDGEISIEGATFQDTTSAVMTG |
| | HKMYNAKFSYYWFDYVTDDPLKKYIDRVEDMKNYITVHPHILEHAQVKIIPL |
| | IPVEINNITELLQYERDVLSKGFEGVMIRKPDGKYKFGRSTLKEGILLKMKQF |
| | KDAEATIISMTALFKNTNTKTKDNFGYSKRSTHKSGKVEEDVMGSIEVDYDG |
| | VVFSIGTGFDADQRRDFWQNKESYIGKMVKFKYFEMGSKDCPRFPVFIGIRH |
| | EEDRSGGSKRTADGSEFESPKKKRKV (SEQ ID NO: 125) |
| PCV2- | MSPSKKNGRSGPQPHKRWVFTLNNPSEDERKKIRDLPISLFDYFIVGEEGNEE |
| XTEN- | GRTPHLQGFANFVKKQTFNKVKWYLGARCHIEKAKGTDQQNKEYCSKEGN |
| nCas9 | LLMECGAPRSQGQRSGGSSGSETPGTSESATPESSGGSDKKYSIGLDIGTNSV |
| (H840A)- | GWAVITDEYKVPSKKFKVLGNTDRHSIKKNLIGALLFDSGETAEATRLKRTA |
| SGGS | RRRYTRRKNRICYLQEIFSNEMAKVDDSFFHRLEESFLVEEDKKHERHPIFGN |
| (SEQ ID | IVDEVAYHEKYPTIYHLRKKLVDSTDKADLRLIYLALAHMIKFRGHFLIEGDL |
| NO: | NPDNSDVDKLFIQLVQTYNQLFEENPINASGVDAKAILSARLSKSRRLENLIA |
| 573)_ | QLPGEKKNGLFGNLIALSLGLTPNFKSNFDLAEDAKLQLSKDTYDDDLDNLL |
| NLS_ | AQIGDQYADLFLAAKNLSDAILLSDILRVNTEITKAPLSASMIKRYDEHHQDL |
| SGGS | TLLKALVRQQLPEKYKEIFFDQSKNGYAGYIDGGASQEEFYKFIKPILEKMDG |
| (SEQ ID | TEELLVKLNREDLLRKQRTFDNGSIPHQIHLGELHAILRRQEDFYPFLKDNRE |
| NO: | KIEKILTFRIPYYVGPLARGNSRFAWMTRKSEETITPWNFEEVVDKGASAQSF |
| 573)- | IERMTNFDKNLPNEKVLPKHSLLYEYFTVYNELTKVKYVTEGMRKPAFLSGE |
| PhiKMVLig- | QKKAIVDLLFKTNRKVTVKQLKEDYFKKIECFDSVEISGVEDRFNASLGTYH |
| NLS | DLLKIIKDKDFLDNEENEDILEDIVLTLTLFEDREMIEERLKTYAHLFDDKVM |
| | KQLKRRRYTGWGRLSRKLINGIRDKQSGKTILDFLKSDGFANRNFMQLIHDD |
| | SLTFKEDIQKAQVSGQGDSLHEHIANLAGSPAIKKGILQTVKVVDELVKVMG |
| | RHKPENIVIEMARENQTTQKGQKNSRERMKRIEEGIKELGSQILKEHPVENTQ |
| | LQNEKLYLYYLQNGRDMYVDQELDINRLSDYDVDAIVPQSFLKDDSIDNKV |
| | LTRSDKNRGKSDNVPSEEVVKKMKNYWRQLLNAKLITQRKFDNLTKAERG |
| | GLSELDKAGFIKRQLVETRQITKHVAQILDSRMNTKYDENDKLIREVKVITLK |
| | SKLVSDFRKDFQFYKVREINNYHHAHDAYLNAVVGTALIKKYPKLESEFVY |
| | GDYKVYDVRKMIAKSEQEIGKATAKYFFYSNIMNFFKTEITLANGEIRKRPLI |
| | ETNGETGEIVWDKGRDFATVRKVLSMPQVNIVKKTEVQTGGFSKESILPKRN |
| | SDKLIARKKDWDPKKYGGFDSPTVAYSVLVVAKVEKGKSKKLKSVKELLGI |
| | TIMERSSFEKNPIDFLEAKGYKEVKKDLIIKLPKYSLFELENGRKRMLASAGE |
| | LQKGNELALPSKYVNFLYLASHYEKLKGSPEDNEQKQLFVEQHKHYLDEDIE |
| | QISEFSKRVILADANLDKVLSAYNKHRDKPIREQAENIIHLFTLTNLGAPAAFK |
| | YFDTTIDRKRYTSTKEVLDATLIHQSITGLYETRIDLSQLGGDSGGSSGGSKRT |
| | ADGSEFESPKKKRKVSGGSSGGSSKRDVVLDIEKGIWRGVDQNDKAVEAIIK |
| | KNGYVIVEPKIDGCRAIVGAHGVVSRSGRRFPALDGLEDRIIAKLSQSGLDSG |
| | LVLDCEMYLEGMPFSEATGRMARKTPLTKAELKCLHFAVFDATHIGVLRKS |
| | RKSHLVYDERRAMVRSLMEDCRRGDTPYFFQVAAQSCRSMEAVLRWYGYH |
| | RAMGFEGSMEKDPSLTYRNGKVAGCYKRKPEITVDGRIVGYVMGKTGKNV |
| | GRVVGYRVELEDGSGTVAATGLSEEHIQLLTCAYLNAHIDEAMPNYGRIVEV |
| | SAMERSANTLRHPSFSRFRDLASNPGVKVSGGSKRTADGSEFESPKKKRKV |
| | (SEQ ID NO: 126) |
| PCV2- | MSPSKKNGRSGPQPHKRWVFTLNNPSEDERKKIRDLPISLFDYFIVGEEGNEE |
| XTEN- | GRTPHLQGFANFVKKQTFNKVKWYLGARCHIEKAKGTDQQNKEYCSKEGN |
| nCas9 | LLMECGAPRSQGQRSGGSSGSETPGTSESATPESSGGSDKKYSIGLDIGTNSV |
| (H840A)- | GWAVITDEYKVPSKKFKVLGNTDRHSIKKNLIGALLFDSGETAEATRLKRTA |
| SGGS | RRRYTRRKNRICYLQEIFSNEMAKVDDSFFHRLEESFLVEEDKKHERHPIFGN |
| (SEQ ID | IVDEVAYHEKYPTIYHLRKKLVDSTDKADLRLIYLALAHMIKFRGHFLIEGDL |
| NO: | NPDNSDVDKLFIQLVQTYNQLFEENPINASGVDAKAILSARLSKSRRLENLIA |
| 573)_ | QLPGEKKNGLFGNLIALSLGLTPNFKSNFDLAEDAKLQLSKDTYDDDLDNLL |
| NLS_ | AQIGDQYADLFLAAKNLSDAILLSDILRVNTEITKAPLSASMIKRYDEHHQDL |
| SGGS | TLLKALVRQQLPEKYKEIFFDQSKNGYAGYIDGGASQEEFYKFIKPILEKMDG |
| (SEQ ID | TEELLVKLNREDLLRKQRTFDNGSIPHQIHLGELHAILRRQEDFYPFLKDNRE |
| NO: | KIEKILTFRIPYYVGPLARGNSRFAWMTRKSEETITPWNFEEVVDKGASAQSF |
| 573)- | IERMTNFDKNLPNEKVLPKHSLLYEYFTVYNELTKVKYVTEGMRKPAFLSGE |
| VacciniaLig- | QKKAIVDLLFKTNRKVTVKQLKEDYFKKIECFDSVEISGVEDRFNASLGTYH |
| NLS | DLLKIIKDKDFLDNEENEDILEDIVLTLTLFEDREMIEERLKTYAHLFDDKVM |
| | KQLKRRRYTGWGRLSRKLINGIRDKQSGKTILDFLKSDGFANRNFMQLIHDD |
| | SLTFKEDIQKAQVSGQGDSLHEHIANLAGSPAIKKGILQTVKVVDELVKVMG |
| | RHKPENIVIEMARENQTTQKGQKNSRERMKRIEEGIKELGSQILKEHPVENTQ |
| | LQNEKLYLYYLQNGRDMYVDQELDINRLSDYDVDAIVPQSFLKDDSIDNKV |
| | LTRSDKNRGKSDNVPSEEVVKKMKNYWRQLLNAKLITQRKFDNLTKAERG |
| | GLSELDKAGFIKRQLVETRQITKHVAQILDSRMNTKYDENDKLIREVKVITLK |
| | SKLVSDFRKDFQFYKVREINNYHHAHDAYLNAVVGTALIKKYPKLESEFVY |
| | GDYKVYDVRKMIAKSEQEIGKATAKYFFYSNIMNFFKTEITLANGEIRKRPLI |
| | ETNGETGEIVWDKGRDFATVRKVLSMPQVNIVKKTEVQTGGFSKESILPKRN |
| | SDKLIARKKDWDPKKYGGFDSPTVAYSVLVVAKVEKGKSKKLKSVKELLGI |
| | TIMERSSFEKNPIDFLEAKGYKEVKKDLIIKLPKYSLFELENGRKRMLASAGE |
| | LQKGNELALPSKYVNFLYLASHYEKLKGSPEDNEQKQLFVEQHKHYLDEIIE |
| | QISEFSKRVILADANLDKVLSAYNKHRDKPIREQAENIIHLFTLTNLGAPAAFK |
| | YFDTTIDRKRYTSTKEVLDATLIHQSITGLYETRIDLSQLGGDSGGSSGGSKRT |
| | ADGSEFESPKKKRKVSGGSSGGSTSLREFRKLCCDIYHASGYKEKSKLIRDFIT |
| | DRDDKYLIIKLLLPGLDDRIYNMNDKQIIKLYSIIFKQSQEDMLQDLGYGYIG |
| | DTIRTFFKENTEIRPRDKSILTLEDVDSFLTTLSSVTKESHQIKLLTDIASVCTC |
| | NDLKCVVMLIDKDLKIKAGPRYVLNAISPNAYDVFRKSNNLKEIIENASKQN |
| | LDSISISVMTPINPMLAESCDSVNKAFKKFPSGMFAEVKYDGERVQVHKNNN |
| | EFAFFSRNMKPVLSHKVDYLKEYIPKAFKKATSIVLDSEIVLVDEHNVPLPFG |
| | SLGIHKKKEYKNSNMCLFVFDCLYFDGFDMTDIPLYERRSFLKDVMVEIPNRI |
| | VFSELTNISNESQLTDVLDDALTRKLEGLVLKDINGVYEPGKRRWLKIKRDY |
| | LNEGSMADSADLVVLGAYYGKGAKGGIMAVFLMGCYDDESGKWKTVTKC |
| | SGHDDNTLRVLQDQLTMVKINKDPKKIPEWLVVNKIYIPDFVVEDPKQSQIW |
| | EISGAEFTSSKSHTANGISIRFPRFTRIREDKTWKESTHLNDLVNLTKSSGGSK |
| | RTADGSEFESPKKKRKV (SEQ ID NO: 127) |
| NLS- | MKRTADGSEFESPKKKRKVSGGSDKKYSIGLDIGTNSVGWAVITDEYKVPSK |
| nCas9 | KFKVLGNTDRHSIKKNLIGALLFDSGETAEATRLKRTARRRYTRRKNRICYL |
| (H840A)- | QEIFSNEMAKVDDSFFHRLEESFLVEEDKKHERHPIFGNIVDEVAYHEKYPTI |
| SGGS | YHLRKKLVDSTDKADLRLIYLALAHMIKFRGHFLIEGDLNPDNSDVDKLFIQ |
| (SEQ ID | LVQTYNQLFEENPINASGVDAKAILSARLSKSRRLENLIAQLPGEKKNGLFGN |
| NO: | LIALSLGLTPNFKSNFDLAEDAKLQLSKDTYDDDLDNLLAQIGDQYADLFLA |
| 573)_ | AKNLSDAILLSDILRVNTEITKAPLSASMIKRYDEHHQDLTLLKALVRQQLPE |
| NLS_ | KYKEIFFDQSKNGYAGYIDGGASQEEFYKFIKPILEKMDGTEELLVKLNREDL |
| SGGS | LRKQRTFDNGSIPHQIHLGELHAILRRQEDFYPFLKDNREKIEKILTFRIPYYV |
| (SEQ ID | GPLARGNSRFAWMTRKSEETITPWNFEEVVDKGASAQSFIERMTNFDKNLPN |
| NO: | EKVLPKHSLLYEYFTVYNELTKVKYVTEGMRKPAFLSGEQKKAIVDLLFKTN |
| 573)- | RKVTVKQLKEDYFKKIECFDSVEISGVEDRFNASLGTYHDLLKIIKDKDFLDN |
| T4Lig- | EENEDILEDIVLTLTLFEDREMIEERLKTYAHLFDDKVMKQLKRRRYTGWGR |
| NLS | LSRKLINGIRDKQSGKTILDFLKSDGFANRNFMQLIHDDSLTFKEDIQKAQVS |
| | GQGDSLHEHIANLAGSPAIKKGILQTVKVVDELVKVMGRHKPENIVIEMARE |
| | NQTTQKGQKNSRERMKRIEEGIKELGSQILKEHPVENTQLQNEKLYLYYLQN |
| | GRDMYVDQELDINRLSDYDVDAIVPQSFLKDDSIDNKVLTRSDKNRGKSDN |
| | VPSEEVVKKMKNYWRQLLNAKLITQRKFDNLTKAERGGLSELDKAGFIKRQ |
| | LVETRQITKHVAQILDSRMNTKYDENDKLIREVKVITLKSKLVSDFRKDFQFY |
| | KVREINNYHHAHDAYLNAVVGTALIKKYPKLESEFVYGDYKVYDVRKMIA |
| | KSEQEIGKATAKYFFYSNIMNFFKTEITLANGEIRKRPLIETNGETGEIVWDKG |
| | RDFATVRKVLSMPQVNIVKKTEVQTGGFSKESILPKRNSDKLIARKKDWDPK |
| | KYGGFDSPTVAYSVLVVAKVEKGKSKKLKSVKELLGITIMERSSFEKNPIDFL |
| | EAKGYKEVKKDLIIKLPKYSLFELENGRKRMLASAGELQKGNELALPSKYVN |
| | FLYLASHYEKLKGSPEDNEQKQLFVEQHKHYLDEIIEQISEFSKRVILADANL |
| | DKVLSAYNKHRDKPIREQAENIIHLFTLTNLGAPAAFKYFDTTIDRKRYTSTK |
| | EVLDATLIHQSITGLYETRIDLSQLGGDSGGSSGGSKRTADGSEFESPKKKRK |
| | VSGGSSGGSILKILNEIASIGSTKQKQAILEKNKDNELLKRVYRLTYSRGLQY |
| | YIKKWPKPGIATQSFGMLTLTDMLDFIEFTLATRKLTGNAAIEELTGYITDGK |
| | KDDVEVLRRVMMRDLECGASVSIANKVWPGLIPEQPQMLASSYDEKGINKN |
| | IKFPAFAQLKADGARCFAEVRGDELDDVRLLSRAGNEYLGLDLLKEELIKMT |
| | AEARQIHPEGVLIDGELVYHEQVKKEPEGLDFLFDAYPENSKAKEFAEVAES |
| | RTASNGIANKSLKGTISEKEAQCMKFQVWDYVPLVEIYSLPAFRLKYDVRFS |
| | KLEQMTSGYDKVILIENQVVNNLDEAKVIYKKYIDQGLEGIILKNIDGLWEN |
| | ARSKNLYKFKEVIDVDLKIVGIYPHRKDPTKAGGFILESECGKIKVNAGSGLK |
| | DKAGVKSHELDRTRIMENQNYYIGKILECECNGWLKSDGRTDYVKLFLPIAI |
| | RLREDKTKANTFEDVFGDFHEVTGLSGGSKRTADGSEFESPKKKRKV (SEQ |
| | ID NO: 128) |
| mSA_H- | MAEAGITGTWYNQHGSTFTVTAGADGNLTGQYENRAQGTGCQNSPYTLTG |
| XTEN- | RYNGTKLEWRVEWNNSTENCHSRTEWRGQYQGGAEARINTQWNLTYEGGS |
| nCas9 | GPATEQGQDTFTKVKPSAASSGGSSGSETPGTSESATPESSGGSDKKYSIGLDI |
| (H840A, | GTNSVGWAVITDEYKVPSKKFKVLGNTDRHSIKKNLIGALLFDSGETAEATR |
| R221K, | LKRTARRRYTRRKNRICYLQEIFSNEMAKVDDSFFHRLEESFLVEEDKKHER |
| N394K)- | HPIFGNIVDEVAYHEKYPTIYHLRKKLVDSTDKADLRLIYLALAHMIKFRGHF |
| SGGS | LIEGDLNPDNSDVDKLFIQLVQTYNQLFEENPINASGVDAKAILSARLSKSRK |
| (SEQ ID | LENLIAQLPGEKKNGLFGNLIALSLGLTPNFKSNFDLAEDAKLQLSKDTYDD |
| NO: | DLDNLLAQIGDQYADLFLAAKNLSDAILLSDILRVNTEITKAPLSASMIKRYD |
| 573)_ | EHHQDLTLLKALVRQQLPEKYKEIFFDQSKNGYAGYIDGGASQEEFYKFIKPI |
| NLS_ | LEKMDGTEELLVKLKREDLLRKQRTFDNGSIPHQIHLGELHAILRRQEDFYPF |
| SGGS | LKDNREKIEKILTFRIPYYVGPLARGNSRFAWMTRKSEETITPWNFEEVVDKG |
| (SEQ ID | ASAQSFIERMTNFDKNLPNEKVLPKHSLLYEYFTVYNELTKVKYVTEGMRKP |
| NO: | AFLSGEQKKAIVDLLFKTNRKVTVKQLKEDYFKKIECFDSVEISGVEDRFNAS |
| 573)- | LGTYHDLLKIIKDKDFLDNEENEDILEDIVLTLTLFEDREMIEERLKTYAHLFD |
| T4Lig- | DKVMKQLKRRRYTGWGRLSRKLINGIRDKQSGKTILDFLKSDGFANRNFMQ |
| NLS | LIHDDSLTFKEDIQKAQVSGQGDSLHEHIANLAGSPAIKKGILQTVKVVDELV |
| | KVMGRHKPENIVIEMARENQTTQKGQKNSRERMKRIEEGIKELGSQILKEHP |
| | VENTQLQNEKLYLYYLQNGRDMYVDQELDINRLSDYDVDAIVPQSFLKDDS |
| | IDNKVLTRSDKNRGKSDNVPSEEVVKKMKNYWRQLLNAKLITQRKFDNLTK |
| | AERGGLSELDKAGFIKRQLVETRQITKHVAQILDSRMNTKYDENDKLIREVK |
| | VITLKSKLVSDFRKDFQFYKVREINNYHHAHDAYLNAVVGTALIKKYPKLES |
| | EFVYGDYKVYDVRKMIAKSEQEIGKATAKYFFYSNIMNFFKTEITLANGEIR |
| | KRPLIETNGETGEIVWDKGRDFATVRKVLSMPQVNIVKKTEVQTGGFSKESIL |
| | PKRNSDKLIARKKDWDPKKYGGFDSPTVAYSVLVVAKVEKGKSKKLKSVK |
| | ELLGITIMERSSFEKNPIDFLEAKGYKEVKKDLIIKLPKYSLFELENGRKRMLA |
| | SAGELQKGNELALPSKYVNFLYLASHYEKLKGSPEDNEQKQLFVEQHKHYL |
| | DEIIEQISEFSKRVILADANLDKVLSAYNKHRDKPIREQAENIIHLFTLTNLGAP |
| | AAFKYFDTTIDRKRYTSTKEVLDATLIHQSITGLYETRIDLSQLGGDSGGSSG |
| | GSKRTADGSEFESPKKKRKVSGGSSGGSILKILNEIASIGSTKQKQAILEKNKD |
| | NELLKRVYRLTYSRGLQYYIKKWPKPGIATQSFGMLTLTDMLDFIEFTLATR |
| | KLTGNAAIEELTGYITDGKKDDVEVLRRVMMRDLECGASVSIANKVWPGLIP |
| | EQPQMLASSYDEKGINKNIKFPAFAQLKADGARCFAEVRGDELDDVRLLSRA |
| | GNEYLGLDLLKEELIKMTAEARQIHPEGVLIDGELVYHEQVKKEPEGLDFLF |
| | DAYPENSKAKEFAEVAESRTASNGIANKSLKGTISEKEAQCMKFQVWDYVP |
| | LVEIYSLPAFRLKYDVRFSKLEQMTSGYDKVILIENQVVNNLDEAKVIYKKYI |
| | DQGLEGIILKNIDGLWENARSKNLYKFKEVIDVDLKIVGIYPHRKDPTKAGGF |
| | ILESECGKIKVNAGSGLKDKAGVKSHELDRTRIMENQNYYIGKILECECNGW |
| | LKSDGRTDYVKLFLPIAIRLREDKTKANTFEDVFGDFHEVTGLSGGSKRTAD |
| | GSEFESPKKKRKV (SEQ ID NO: 129) |
| PCV2- | MSPSKKNGRSGPQPHKRWVFTLNNPSEDERKKIRDLPISLFDYFIVGEEGNEE |
| XTEN- | GRTPHLQGFANFVKKQTFNKVKWYLGARCHIEKAKGTDQQNKEYCSKEGN |
| nCas9 | LLMECGAPRSQGQRSGGSSGSETPGTSESATPESSGGSDKKYSIGLDIGTNSV |
| (R221K, | GWAVITDEYKVPSKKFKVLGNTDRHSIKKNLIGALLFDSGETAEATRLKRTA |
| N394K, | RRRYTRRKNRICYLQEIFSNEMAKVDDSFFHRLEESFLVEEDKKHERHPIFGN |
| H840A)- | IVDEVAYHEKYPTIYHLRKKLVDSTDKADLRLIYLALAHMIKFRGHFLIEGDL |
| SGGS | NPDNSDVDKLFIQLVQTYNQLFEENPINASGVDAKAILSARLSKSRKLENLIA |
| (SEQ ID | QLPGEKKNGLFGNLIALSLGLTPNFKSNFDLAEDAKLQLSKDTYDDDLDNLL |
| NO: | AQIGDQYADLFLAAKNLSDAILLSDILRVNTEITKAPLSASMIKRYDEHHQDL |
| 573)_ | TLLKALVRQQLPEKYKEIFFDQSKNGYAGYIDGGASQEEFYKFIKPILEKMDG |
| NLS_ | TEELLVKLKREDLLRKQRTFDNGSIPHQIHLGELHAILRRQEDFYPFLKDNRE |
| SGGS | KIEKILTFRIPYYVGPLARGNSRFAWMTRKSEETITPWNFEEVVDKGASAQSF |
| (SEQ ID | IERMTNFDKNLPNEKVLPKHSLLYEYFTVYNELTKVKYVTEGMRKPAFLSGE |
| NO: | QKKAIVDLLFKTNRKVTVKQLKEDYFKKIECFDSVEISGVEDRFNASLGTYH |
| 573)- | DLLKIIKDKDFLDNEENEDILEDIVLTLTLFEDREMIEERLKTYAHLFDDKVM |
| dT4Lig- | KQLKRRRYTGWGRLSRKLINGIRDKQSGKTILDFLKSDGFANRNFMQLIHDD |
| NLS | SLTFKEDIQKAQVSGQGDSLHEHIANLAGSPAIKKGILQTVKVVDELVKVMG |
| | RHKPENIVIEMARENQTTQKGQKNSRERMKRIEEGIKELGSQILKEHPVENTQ |
| | LQNEKLYLYYLQNGRDMYVDQELDINRLSDYDVDAIVPQSFLKDDSIDNKV |
| | LTRSDKNRGKSDNVPSEEVVKKMKNYWRQLLNAKLITQRKFDNLTKAERG |
| | GLSELDKAGFIKRQLVETRQITKHVAQILDSRMNTKYDENDKLIREVKVITLK |
| | SKLVSDFRKDFQFYKVREINNYHHAHDAYLNAVVGTALIKKYPKLESEFVY |
| | GDYKVYDVRKMIAKSEQEIGKATAKYFFYSNIMNFFKTEITLANGEIRKRPLI |
| | ETNGETGEIVWDKGRDFATVRKVLSMPQVNIVKKTEVQTGGFSKESILPKRN |
| | SDKLIARKKDWDPKKYGGFDSPTVAYSVLVVAKVEKGKSKKLKSVKELLGI |
| | TIMERSSFEKNPIDFLEAKGYKEVKKDLIIKLPKYSLFELENGRKRMLASAGE |
| | LQKGNELALPSKYVNFLYLASHYEKLKGSPEDNEQKQLFVEQHKHYLDEIIE |
| | QISEFSKRVILADANLDKVLSAYNKHRDKPIREQAENIIHLFTLTNLGAPAAFK |
| | YFDTTIDRKRYTSTKEVLDATLIHQSITGLYETRIDLSQLGGDSGGSSGGSKRT |
| | ADGSEFESPKKKRKVSGGSSGGSILKILNEIASIGSTKQKQAILEKNKDNELLK |
| | RVYRLTYSRGLQYYIKKWPKPGIATQSFGMLTLTDMLDFIEFTLATRKLTGN |
| | AAIEELTGYITDGKKDDVEVLRRVMMRDLECGASVSIANKVWPGLIPEQPQ |
| | MLASSYDEKGINKNIKFPAFAQLAADGAACFAEVRGDELDDVRLLSRAGNE |
| | YLGLDLLKEELIKMTAEARQIHPEGVLIDGELVYHEQVKKEPEGLDFLFDAY |
| | PENSKAKEFAEVAESRTASNGIANKSLKGTISEKEAQCMKFQVWDYVPLVEI |
| | YSLPAFRLKYDVRFSKLEQMTSGYDKVILIENQVVNNLDEAKVIYKKYIDQG |
| | LEGIILKNIDGLWENARSKNLYKFKEVIDVDLKIVGIYPHRKDPTKAGGFILES |
| | ECGKIKVNAGSGLKDKAGVKSHELDRTRIMENQNYYIGKILECECNGWLKS |
| | DGRTDYVKLFLPIAIRLREDKTKANTFEDVFGDFHEVTGLSGGSKRTADGSEF |
| | ESPKKKRKV (SEQ ID NO: 130) |

Exemplary Full PCE amino acid sequences

| name | aa sequence |
|---|---|
| PCV2- | MSPSKKNGRSGPQPHKRWVFTLNNPSEDERKKIRDLPISLFDYFIVGEEGNEE |
| XTEN- | GRTPHLQGFANFVKKQTFNKVKWYLGARCHIEKAKGTDQQNKEYCSKEGN |
| nCas9 | LLMECGAPRSQGQRSGGSSGSETPGTSESATPESSGGSDKKYSIGLDIGTNSV |
| (H840A)- | GWAVITDEYKVPSKKFKVLGNTDRHSIKKNLIGALLFDSGETAEATRLKRTA |
| SGGS | RRRYTRRKNRICYLQEIFSNEMAKVDDSFFHRLEESFLVEEDKKHERHPIFGN |
| (SEQ ID | IVDEVAYHEKYPTIYHLRKKLVDSTDKADLRLIYLALAHMIKFRGHFLIEGDL |
| NO: | NPDNSDVDKLFIQLVQTYNQLFEENPINASGVDAKAILSARLSKSRRLENLIA |
| 573)_ | QLPGEKKNGLFGNLIALSLGLTPNFKSNFDLAEDAKLQLSKDTYDDDLDNLL |
| NLS_ | AQIGDQYADLFLAAKNLSDAILLSDILRVNTEITKAPLSASMIKRYDEHHQDL |
| SGGS | TLLKALVRQQLPEKYKEIFFDQSKNGYAGYIDGGASQEEFYKFIKPILEKMDG |
| (SEQ ID | TEELLVKLNREDLLRKQRTFDNGSIPHQIHLGELHAILRRQEDFYPFLKDNRE |
| NO: | KIEKILTFRIPYYVGPLARGNSRFAWMTRKSEETITPWNFEEVVDKGASAQSF |
| 573)- | IERMTNFDKNLPNEKVLPKHSLLYEYFTVYNELTKVKYVTEGMRKPAFLSGE |
| EcKLenow | QKKAIVDLLFKTNRKVTVKQLKEDYFKKIECFDSVEISGVEDRFNASLGTYH |
| (D355A, | DLLKIIKDKDFLDNEENEDILEDIVLTLTLFEDREMIEERLKTYAHLFDDKVM |
| D357A)- | KQLKRRRYTGWGRLSRKLINGIRDKQSGKTILDFLKSDGFANRNFMQLIHDD |
| NLS | SLTFKEDIQKAQVSGQGDSLHEHIANLAGSPAIKKGILQTVKVVDELVKVMG |
| | RHKPENIVIEMARENQTTQKGQKNSRERMKRIEEGIKELGSQILKEHPVENTQ |
| | LQNEKLYLYYLQNGRDMYVDQELDINRLSDYDVDAIVPQSFLKDDSIDNKV |
| | LTRSDKNRGKSDNVPSEEVVKKMKNYWRQLLNAKLITQRKFDNLTKAERG |
| | GLSELDKAGFIKRQLVETRQITKHVAQILDSRMNTKYDENDKLIREVKVITLK |
| | SKLVSDFRKDFQFYKVREINNYHHAHDAYLNAVVGTALIKKYPKLESEFVY |
| | GDYKVYDVRKMIAKSEQEIGKATAKYFFYSNIMNFFKTEITLANGEIRKRPLI |
| | ETNGETGEIVWDKGRDFATVRKVLSMPQVNIVKKTEVQTGGFSKESILPKRN |
| | SDKLIARKKDWDPKKYGGFDSPTVAYSVLVVAKVEKGKSKKLKSVKELLGI |
| | TIMERSSFEKNPIDFLEAKGYKEVKKDLIIKLPKYSLFELENGRKRMLASAGE |
| | LQKGNELALPSKYVNFLYLASHYEKLKGSPEDNEQKQLFVEQHKHYLDEIIE |
| | QISEFSKRVILADANLDKVLSAYNKHRDKPIREQAENIIHLFTLTNLGAPAAFK |
| | YFDTTIDRKRYTSTKEVLDATLIHQSITGLYETRIDLSQLGGDSGGSSGGSKRT |
| | ADGSEFESPKKKRKVSGGSSGGSVISYDNYVTILDEETLKAWIAKLEKAPVFA |
| | FATATDSLDNISANLVGLSFAIEPGVAAYIPVAHDYLDAPDQISRERALELLKP |
| | LLEDEKALKVGQNLKYDRGILANYGIELRGIAFDTMLESYILNSVAGRHDMD |
| | SLAERWLKHKTITFEEIAGKGKNQLTFNQIALEEAGRYAAEDADVTLQLHLK |
| | MWPDLQKHKGPLNVFENIEMPLVPVLSRIERNGVKIDPKVLHNHSEELTLRL |
| | AELEKKAHEIAGEEFNLSSTKQLQTILFEKQGIKPLKKTPGGAPSTSEEVLEEL |
| | ALDYPLPKVILEYRGLAKLKSTYTDKLPLMINPKTGRVHTSYHQAVTATGRL |
| | SSTDPNLQNIPVRNEEGRRIRQAFIAPEDYVIVSADYSQIELRIMAHLSRDKGL |
| | LTAFAEGKDIHRATAAEVFGLPLETVTSEQRRSAKAINFGLIYGMSAFGLARQ |
| | LNIPRKEAQKYMDLYFERYPGVLEYMERTRAQAKEQGYVETLDGRRLYLPD |
| | IKSSNGARRAAAERAAINAPMQGTAADIIKRAMIAVDAWLQAEQPRVRMIM |
| | QVHDELVFEVHKDDVDAVAKQIHQLMENCTRLDVPLLVEVGSGENWDQAH |
| | SGGSKRTADGSEFESPKKKRKV (SEQ ID NO: 131) |
| PCV2- | MSPSKKNGRSGPQPHKRWVFTLNNPSEDERKKIRDLPISLFDYFIVGEEGNEE |
| XTEN- | GRTPHLQGFANFVKKQTFNKVKWYLGARCHIEKAKGTDQQNKEYCSKEGN |
| nCas9 | LLMECGAPRSQGQRSGGSSGSETPGTSESATPESSGGSDKKYSIGLDIGTNSV |
| (H840A)- | GWAVITDEYKVPSKKFKVLGNTDRHSIKKNLIGALLFDSGETAEATRLKRTA |
| SGGS | RRRYTRRKNRICYLQEIFSNEMAKVDDSFFHRLEESFLVEEDKKHERHPIFGN |
| (SEQ ID | IVDEVAYHEKYPTIYHLRKKLVDSTDKADLRLIYLALAHMIKFRGHFLIEGDL |
| NO: | NPDNSDVDKLFIQLVQTYNQLFEENPINASGVDAKAILSARLSKSRRLENLIA |
| 573)_ | QLPGEKKNGLFGNLIALSLGLTPNFKSNFDLAEDAKLQLSKDTYDDDLDNLL |
| NLS_ | AQIGDQYADLFLAAKNLSDAILLSDILRVNTEITKAPLSASMIKRYDEHHQDL |
| SGGS | TLLKALVRQQLPEKYKEIFFDQSKNGYAGYIDGGASQEEFYKFIKPILEKMDG |
| (SEQ ID | TEELLVKLNREDLLRKQRTFDNGSIPHQIHLGELHAILRRQEDFYPFLKDNRE |
| NO: | KIEKILTFRIPYYVGPLARGNSRFAWMTRKSEETITPWNFEEVVDKGASAQSF |
| 573)- | IERMTNFDKNLPNEKVLPKHSLLYEYFTVYNELTKVKYVTEGMRKPAFLSGE |
| dEcKLenow | QKKAIVDLLFKTNRKVTVKQLKEDYFKKIECFDSVEISGVEDRFNASLGTYH |
| (D355A, | DLLKIIKDKDFLDNEENEDILEDIVLTLTLFEDREMIEERLKTYAHLFDDKVM |
| D357A, | KQLKRRRYTGWGRLSRKLINGIRDKQSGKTILDFLKSDGFANRNFMQLIHDD |
| D705A, | SLTFKEDIQKAQVSGQGDSLHEHIANLAGSPAIKKGILQTVKVVDELVKVMG |
| D882A)-NLS | RHKPENIVIEMARENQTTQKGQKNSRERMKRIEEGIKELGSQILKEHPVENTQ |
| | LQNEKLYLYYLQNGRDMYVDQELDINRLSDYDVDAIVPQSFLKDDSIDNKV |
| | LTRSDKNRGKSDNVPSEEVVKKMKNYWRQLLNAKLITQRKFDNLTKAERG |
| | GLSELDKAGFIKRQLVETRQITKHVAQILDSRMNTKYDENDKLIREVKVITLK |
| | SKLVSDFRKDFQFYKVREINNYHHAHDAYLNAVVGTALIKKYPKLESEFVY |
| | GDYKVYDVRKMIAKSEQEIGKATAKYFFYSNIMNFFKTEITLANGEIRKRPLI |
| | ETNGETGEIVWDKGRDFATVRKVLSMPQVNIVKKTEVQTGGFSKESILPKRN |
| | SDKLIARKKDWDPKKYGGFDSPTVAYSVLVVAKVEKGKSKKLKSVKELLGI |
| | TIMERSSFEKNPIDFLEAKGYKEVKKDLIIKLPKYSLFELENGRKRMLASAGE |
| | LQKGNELALPSKYVNFLYLASHYEKLKGSPEDNEQKQLFVEQHKHYLDEDIE |
| | QISEFSKRVILADANLDKVLSAYNKHRDKPIREQAENIIHLFTLTNLGAPAAFK |
| | YFDTTIDRKRYTSTKEVLDATLIHQSITGLYETRIDLSQLGGDSGGSSGGSKRT |
| | ADGSEFESPKKKRKVSGGSSGGSVISYDNYVTILDEETLKAWIAKLEKAPVFA |
| | FATATDSLDNISANLVGLSFAIEPGVAAYIPVAHDYLDAPDQISRERALELLKP |
| | LLEDEKALKVGQNLKYDRGILANYGIELRGIAFDTMLESYILNSVAGRHDMD |
| | SLAERWLKHKTITFEEIAGKGKNQLTFNQIALEEAGRYAAEDADVTLQLHLK |
| | MWPDLQKHKGPLNVFENIEMPLVPVLSRIERNGVKIDPKVLHNHSEELTLRL |
| | AELEKKAHEIAGEEFNLSSTKQLQTILFEKQGIKPLKKTPGGAPSTSEEVLEEL |
| | ALDYPLPKVILEYRGLAKLKSTYTDKLPLMINPKTGRVHTSYHQAVTATGRL |
| | SSTDPNLQNIPVRNEEGRRIRQAFIAPEDYVIVSAAYSQIELRIMAHLSRDKGL |
| | LTAFAEGKDIHRATAAEVFGLPLETVTSEQRRSAKAINFGLIYGMSAFGLARQ |
| | LNIPRKEAQKYMDLYFERYPGVLEYMERTRAQAKEQGYVETLDGRRLYLPD |
| | IKSSNGARRAAAERAAINAPMQGTAADIIKRAMIAVDAWLQAEQPRVRMIM |
| | QVHAELVFEVHKDDVDAVAKQIHQLMENCTRLDVPLLVEVGSGENWDQAH |
| | SGGSKRTADGSEFESPKKKRKV (SEQ ID NO: 132) |
| PCV2- | MSPSKKNGRSGPQPHKRWVFTLNNPSEDERKKIRDLPISLFDYFIVGEEGNEE |
| XTEN- | GRTPHLQGFANFVKKQTFNKVKWYLGARCHIEKAKGTDQQNKEYCSKEGN |
| nCas9 | LLMECGAPRSQGQRSGGSSGSETPGTSESATPESSGGSDKKYSIGLDIGTNSV |
| (H840A)- | GWAVITDEYKVPSKKFKVLGNTDRHSIKKNLIGALLFDSGETAEATRLKRTA |
| SGGS | RRRYTRRKNRICYLQEIFSNEMAKVDDSFFHRLEESFLVEEDKKHERHPIFGN |
| (SEQ ID | IVDEVAYHEKYPTIYHLRKKLVDSTDKADLRLIYLALAHMIKFRGHFLIEGDL |
| NO: | NPDNSDVDKLFIQLVQTYNQLFEENPINASGVDAKAILSARLSKSRRLENLIA |
| 573)_ | QLPGEKKNGLFGNLIALSLGLTPNFKSNFDLAEDAKLQLSKDTYDDDLDNLL |
| NLS_ | AQIGDQYADLFLAAKNLSDAILLSDILRVNTEITKAPLSASMIKRYDEHHQDL |
| SGGS | TLLKALVRQQLPEKYKEIFFDQSKNGYAGYIDGGASQEEFYKFIKPILEKMDG |
| (SEQ ID | TEELLVKLNREDLLRKQRTFDNGSIPHQIHLGELHAILRRQEDFYPFLKDNRE |
| NO: | KIEKILTFRIPYYVGPLARGNSRFAWMTRKSEETITPWNFEEVVDKGASAQSF |
| 573)- | IERMTNFDKNLPNEKVLPKHSLLYEYFTVYNELTKVKYVTEGMRKPAFLSGE |
| TaqStoffel- | QKKAIVDLLFKTNRKVTVKQLKEDYFKKIECFDSVEISGVEDRFNASLGTYH |
| NLS | DLLKIIKDKDFLDNEENEDILEDIVLTLTLFEDREMIEERLKTYAHLFDDKVM |
| | KQLKRRRYTGWGRLSRKLINGIRDKQSGKTILDFLKSDGFANRNFMQLIHDD |
| | SLTFKEDIQKAQVSGQGDSLHEHIANLAGSPAIKKGILQTVKVVDELVKVMG |
| | RHKPENIVIEMARENQTTQKGQKNSRERMKRIEEGIKELGSQILKEHPVENTQ |
| | LQNEKLYLYYLQNGRDMYVDQELDINRLSDYDVDAIVPQSFLKDDSIDNKV |
| | LTRSDKNRGKSDNVPSEEVVKKMKNYWRQLLNAKLITQRKFDNLTKAERG |
| | GLSELDKAGFIKRQLVETRQITKHVAQILDSRMNTKYDENDKLIREVKVITLK |
| | SKLVSDFRKDFQFYKVREINNYHHAHDAYLNAVVGTALIKKYPKLESEFVY |
| | GDYKVYDVRKMIAKSEQEIGKATAKYFFYSNIMNFFKTEITLANGEIRKRPLI |
| | ETNGETGEIVWDKGRDFATVRKVLSMPQVNIVKKTEVQTGGFSKESILPKRN |
| | SDKLIARKKDWDPKKYGGFDSPTVAYSVLVVAKVEKGKSKKLKSVKELLGI |
| | TIMERSSFEKNPIDFLEAKGYKEVKKDLIIKLPKYSLFELENGRKRMLASAGE |
| | LQKGNELALPSKYVNFLYLASHYEKLKGSPEDNEQKQLFVEQHKHYLDEIIE |
| | QISEFSKRVILADANLDKVLSAYNKHRDKPIREQAENIIHLFTLTNLGAPAAFK |
| | YFDTTIDRKRYTSTKEVLDATLIHQSITGLYETRIDLSQLGGDSGGSSGGSKRT |
| | ADGSEFESPKKKRKVSGGSSGGSALEEAPWPPPEGAFVGFVLSRKEPMWAD |
| | LLALAAARGGRVHRAPEPYKALRDLKEARGLLAKDLSVLALREGLGLPPGD |
| | DPMLLAYLLDPSNTTPEGVARRYGGEWTEEAGERAALSERLFANLWGRLEG |
| | EERLLWLYREVERPLSAVLAHMEATGVRLDVAYLRALSLEVAEEIARLEAE |
| | VFRLAGHPFNLNSRDQLERVLFDELGLPAIGKTEKTGKRSTSAAVLEALREA |
| | HPIVEKILQYRELTKLKSTYIDPLPDLIHPRTGRLHTRFNQTATATGRLSSSDP |
| | NLQNIPVRTPLGQRIRRAFIAEEGWLLVALDYSQIELRVLAHLSGDENLIRVF |
| | QEGRDIHTETASWMFGVPREAVDPLMRRAAKTINFGVLYGMSAHRLSQELA |
| | IPYEEAQAFIERYFQSFPKVRAWIEKTLEEGRRRGYVETLFGRRRYVPDLEAR |
| | VKSVREAAERMAFNMPVQGTAADLMKLAMVKLFPRLEEMGARMLLQVHD |
| | ELVLEAPKERAEAVARLAKEVMEGVYPLAVPLEVEVGIGEDWLSAKESGGS |
| | KRTADGSEFESPKKKRKV (SEQ ID NO: 133) |
| PCV2- | MSPSKKNGRSGPQPHKRWVFTLNNPSEDERKKIRDLPISLFDYFIVGEEGNEE |
| XTEN- | GRTPHLQGFANFVKKQTFNKVKWYLGARCHIEKAKGTDQQNKEYCSKEGN |
| nCas9 | LLMECGAPRSQGQRSGGSSGSETPGTSESATPESSGGSDKKYSIGLDIGTNSV |
| (H840A)- | GWAVITDEYKVPSKKFKVLGNTDRHSIKKNLIGALLFDSGETAEATRLKRTA |
| SGGS | RRRYTRRKNRICYLQEIFSNEMAKVDDSFFHRLEESFLVEEDKKHERHPIFGN |
| (SEQ ID | IVDEVAYHEKYPTIYHLRKKLVDSTDKADLRLIYLALAHMIKFRGHFLIEGDL |
| NO: | NPDNSDVDKLFIQLVQTYNQLFEENPINASGVDAKAILSARLSKSRRLENLIA |
| 573)_ | QLPGEKKNGLFGNLIALSLGLTPNFKSNFDLAEDAKLQLSKDTYDDDLDNLL |
| NLS_ | AQIGDQYADLFLAAKNLSDAILLSDILRVNTEITKAPLSASMIKRYDEHHQDL |
| SGGS | TLLKALVRQQLPEKYKEIFFDQSKNGYAGYIDGGASQEEFYKFIKPILEKMDG |
| (SEQ ID | TEELLVKLNREDLLRKQRTFDNGSIPHQIHLGELHAILRRQEDFYPFLKDNRE |
| NO: | KIEKILTFRIPYYVGPLARGNSRFAWMTRKSEETITPWNFEEVVDKGASAQSF |
| 573)- | IERMTNFDKNLPNEKVLPKHSLLYEYFTVYNELTKVKYVTEGMRKPAFLSGE |
| MMLV | QKKAIVDLLFKTNRKVTVKQLKEDYFKKIECFDSVEISGVEDRFNASLGTYH |
| RT(PE2)- | DLLKIIKDKDFLDNEENEDILEDIVLTLTLFEDREMIEERLKTYAHLFDDKVM |
| NLS | KQLKRRRYTGWGRLSRKLINGIRDKQSGKTILDFLKSDGFANRNFMQLIHDD |
| | SLTFKEDIQKAQVSGQGDSLHEHIANLAGSPAIKKGILQTVKVVDELVKVMG |
| | RHKPENIVIEMARENQTTQKGQKNSRERMKRIEEGIKELGSQILKEHPVENTQ |
| | LQNEKLYLYYLQNGRDMYVDQELDINRLSDYDVDAIVPQSFLKDDSIDNKV |
| | LTRSDKNRGKSDNVPSEEVVKKMKNYWRQLLNAKLITQRKFDNLTKAERG |
| | GLSELDKAGFIKRQLVETRQITKHVAQILDSRMNTKYDENDKLIREVKVITLK |
| | SKLVSDFRKDFQFYKVREINNYHHAHDAYLNAVVGTALIKKYPKLESEFVY |
| | GDYKVYDVRKMIAKSEQEIGKATAKYFFYSNIMNFFKTEITLANGEIRKRPLI |
| | ETNGETGEIVWDKGRDFATVRKVLSMPQVNIVKKTEVQTGGFSKESILPKRN |
| | SDKLIARKKDWDPKKYGGFDSPTVAYSVLVVAKVEKGKSKKLKSVKELLGI |
| | TIMERSSFEKNPIDFLEAKGYKEVKKDLIIKLPKYSLFELENGRKRMLASAGE |
| | LQKGNELALPSKYVNFLYLASHYEKLKGSPEDNEQKQLFVEQHKHYLDEIIE |
| | QISEFSKRVILADANLDKVLSAYNKHRDKPIREQAENIIHLFTLTNLGAPAAFK |
| | YFDTTIDRKRYTSTKEVLDATLIHQSITGLYETRIDLSQLGGDSGGSSGGSKRT |
| | ADGSEFESPKKKRKVSGGSSGGSTLNIEDEYRLHETSKEPDVSLGSTWLSDFP |
| | QAWAETGGMGLAVRQAPLIIPLKATSTPVSIKQYPMSQEARLGIKPHIQRLLD |
| | QGILVPCQSPWNTPLLPVKKPGTNDYRPVQDLREVNKRVEDIHPTVPNPYNL |
| | LSGLPPSHQWYTVLDLKDAFFCLRLHPTSQPLFAFEWRDPEMGISGQLTWTR |
| | LPQGFKNSPTLFNEALHRDLADFRIQHPDLILLQYVDDLLLAATSELDCQQGT |
| | RALLQTLGNLGYRASAKKAQICQKQVKYLGYLLKEGQRWLTEARKETVMG |
| | QPTPKTPRQLREFLGKAGFCRLFIPGFAEMAAPLYPLTKPGTLFNWGPDQQK |
| | AYQEIKQALLTAPALGLPDLTKPFELFVDEKQGYAKGVLTQKLGPWRRPVA |
| | YLSKKLDPVAAGWPPCLRMVAAIAVLTKDAGKLTMGQPLVILAPHAVEALV |
| | KQPPDRWLSNARMTHYQALLLDTDRVQFGPVVALNPATLLPLPEEGLQHNC |
| | LDILAEAHGTRPDLTDQPLPDADHTWYTDGSSLLQEGQRKAGAAVTTETEVI |
| | WAKALPAGTSAQRAELIALTQALKMAEGKKLNVYTDSRYAFATAHIHGEIY |
| | RRRGWLTSEGKEIKNKDEILALLKALFLPKRLSIIHCPGHQKGHSAEARGNR |
| | MADQAARKAAITETPDTSTLLIENSSPSGGSKRTADGSEFESPKKKRKV (SEQ |
| | ID NO: 134) |
| PCV2- | MSPSKKNGRSGPQPHKRWVFTLNNPSEDERKKIRDLPISLFDYFIVGEEGNEE |
| XTEN- | GRTPHLQGFANFVKKQTFNKVKWYLGARCHIEKAKGTDQQNKEYCSKEGN |
| nCas9 | LLMECGAPRSQGQRSGGSSGSETPGTSESATPESSGGSDKKYSIGLDIGTNSV |
| (H840A)- | GWAVITDEYKVPSKKFKVLGNTDRHSIKKNLIGALLFDSGETAEATRLKRTA |
| SGGS | RRRYTRRKNRICYLQEIFSNEMAKVDDSFFHRLEESFLVEEDKKHERHPIFGN |
| (SEQ ID | IVDEVAYHEKYPTIYHLRKKLVDSTDKADLRLIYLALAHMIKFRGHFLIEGDL |
| NO: | NPDNSDVDKLFIQLVQTYNQLFEENPINASGVDAKAILSARLSKSRRLENLIA |
| 573)_ | QLPGEKKNGLFGNLIALSLGLTPNFKSNFDLAEDAKLQLSKDTYDDDLDNLL |
| NLS_ | AQIGDQYADLFLAAKNLSDAILLSDILRVNTEITKAPLSASMIKRYDEHHQDL |
| SGGS | TLLKALVRQQLPEKYKEIFFDQSKNGYAGYIDGGASQEEFYKFIKPILEKMDG |
| (SEQ ID | TEELLVKLNREDLLRKQRTFDNGSIPHQIHLGELHAILRRQEDFYPFLKDNRE |
| NO: | KIEKILTFRIPYYVGPLARGNSRFAWMTRKSEETITPWNFEEVVDKGASAQSF |
| 573)- | IERMTNFDKNLPNEKVLPKHSLLYEYFTVYNELTKVKYVTEGMRKPAFLSGE |
| PolBeta- | QKKAIVDLLFKTNRKVTVKQLKEDYFKKIECFDSVEISGVEDRFNASLGTYH |
| NLS | DLLKIIKDKDFLDNEENEDILEDIVLTLTLFEDREMIEERLKTYAHLFDDKVM |
| | KQLKRRRYTGWGRLSRKLINGIRDKQSGKTILDFLKSDGFANRNFMQLIHDD |
| | SLTFKEDIQKAQVSGQGDSLHEHIANLAGSPAIKKGILQTVKVVDELVKVMG |
| | RHKPENIVIEMARENQTTQKGQKNSRERMKRIEEGIKELGSQILKEHPVENTQ |
| | LQNEKLYLYYLQNGRDMYVDQELDINRLSDYDVDAIVPQSFLKDDSIDNKV |
| | LTRSDKNRGKSDNVPSEEVVKKMKNYWRQLLNAKLITQRKFDNLTKAERG |
| | GLSELDKAGFIKRQLVETRQITKHVAQILDSRMNTKYDENDKLIREVKVITLK |
| | SKLVSDFRKDFQFYKVREINNYHHAHDAYLNAVVGTALIKKYPKLESEFVY |
| | GDYKVYDVRKMIAKSEQEIGKATAKYFFYSNIMNFFKTEITLANGEIRKRPLI |
| | ETNGETGEIVWDKGRDFATVRKVLSMPQVNIVKKTEVQTGGFSKESILPKRN |
| | SDKLIARKKDWDPKKYGGFDSPTVAYSVLVVAKVEKGKSKKLKSVKELLGI |
| | TIMERSSFEKNPIDFLEAKGYKEVKKDLIIKLPKYSLFELENGRKRMLASAGE |
| | LQKGNELALPSKYVNFLYLASHYEKLKGSPEDNEQKQLFVEQHKHYLDEIIE |
| | QISEFSKRVILADANLDKVLSAYNKHRDKPIREQAENIIHLFTLTNLGAPAAFK |
| | YFDTTIDRKRYTSTKEVLDATLIHQSITGLYETRIDLSQLGGDSGGSSGGSKRT |
| | ADGSEFESPKKKRKVSGGSSGGSSKRKAPQETLNGGITDMLTELANFEKNVS |
| | QAIHKYNAYRKAASVIAKYPHKIKSGAEAKKLPGVGTKIAEKIDEFLATGKL |
| | RKLEKIRQDDTSSSINFLTRVSGIGPSAARKFVDEGIKTLEDLRKNEDKLNHH |
| | QRIGLKYFGDFEKRIPREEMLQMQDIVLNEVKKVDSEYIATVCGSFRRGAESS |
| | GDMDVLLTHPSFTSESTKQPKLLHQVVEQLQKVHFITDTLSKGETKFMGVCQ |
| | LPSKNDEKEYPHRRIDIRLIPKDQYYCGVLYFTGSDIFNKNMRAHALEKGFTI |
| | NEYTIRPLGVTGVAGEPLPVDSEKDIFDYIQWKYREPKDRSESGGSKRTADGS |
| | EFESPKKKRKV (SEQ ID NO: 135) |
| PCV2- | MSPSKKNGRSGPQPHKRWVFTLNNPSEDERKKIRDLPISLFDYFIVGEEGNEE |
| XTEN- | GRTPHLQGFANFVKKQTFNKVKWYLGARCHIEKAKGTDQQNKEYCSKEGN |
| nCas9 | LLMECGAPRSQGQRSGGSSGSETPGTSESATPESSGGSDKKYSIGLDIGTNSV |
| (H840A)- | GWAVITDEYKVPSKKFKVLGNTDRHSIKKNLIGALLFDSGETAEATRLKRTA |
| SGGS | RRRYTRRKNRICYLQEIFSNEMAKVDDSFFHRLEESFLVEEDKKHERHPIFGN |
| (SEQ ID | IVDEVAYHEKYPTIYHLRKKLVDSTDKADLRLIYLALAHMIKFRGHFLIEGDL |
| NO: | NPDNSDVDKLFIQLVQTYNQLFEENPINASGVDAKAILSARLSKSRRLENLIA |
| 573)_ | QLPGEKKNGLFGNLIALSLGLTPNFKSNFDLAEDAKLQLSKDTYDDDLDNLL |
| NLS_ | AQIGDQYADLFLAAKNLSDAILLSDILRVNTEITKAPLSASMIKRYDEHHQDL |
| SGGS | TLLKALVRQQLPEKYKEIFFDQSKNGYAGYIDGGASQEEFYKFIKPILEKMDG |
| (SEQ ID | TEELLVKLNREDLLRKQRTFDNGSIPHQIHLGELHAILRRQEDFYPFLKDNRE |
| NO: | KIEKILTFRIPYYVGPLARGNSRFAWMTRKSEETITPWNFEEVVDKGASAQSF |
| 573)- | IERMTNFDKNLPNEKVLPKHSLLYEYFTVYNELTKVKYVTEGMRKPAFLSGE |
| PolBeta- | QKKAIVDLLFKTNRKVTVKQLKEDYFKKIECFDSVEISGVEDRFNASLGTYH |
| Sso7d- | DLLKIIKDKDFLDNEENEDILEDIVLTLTLFEDREMIEERLKTYAHLFDDKVM |
| NLS | KQLKRRRYTGWGRLSRKLINGIRDKQSGKTILDFLKSDGFANRNFMQLIHDD |
| | SLTFKEDIQKAQVSGQGDSLHEHIANLAGSPAIKKGILQTVKVVDELVKVMG |
| | RHKPENIVIEMARENQTTQKGQKNSRERMKRIEEGIKELGSQILKEHPVENTQ |
| | LQNEKLYLYYLQNGRDMYVDQELDINRLSDYDVDAIVPQSFLKDDSIDNKV |
| | LTRSDKNRGKSDNVPSEEVVKKMKNYWRQLLNAKLITQRKFDNLTKAERG |
| | GLSELDKAGFIKRQLVETRQITKHVAQILDSRMNTKYDENDKLIREVKVITLK |
| | SKLVSDFRKDFQFYKVREINNYHHAHDAYLNAVVGTALIKKYPKLESEFVY |
| | GDYKVYDVRKMIAKSEQEIGKATAKYFFYSNIMNFFKTEITLANGEIRKRPLI |
| | ETNGETGEIVWDKGRDFATVRKVLSMPQVNIVKKTEVQTGGFSKESILPKRN |
| | SDKLIARKKDWDPKKYGGFDSPTVAYSVLVVAKVEKGKSKKLKSVKELLGI |
| | TIMERSSFEKNPIDFLEAKGYKEVKKDLIIKLPKYSLFELENGRKRMLASAGE |
| | LQKGNELALPSKYVNFLYLASHYEKLKGSPEDNEQKQLFVEQHKHYLDEIIE |
| | QISEFSKRVILADANLDKVLSAYNKHRDKPIREQAENIIHLFTLTNLGAPAAFK |
| | YFDTTIDRKRYTSTKEVLDATLIHQSITGLYETRIDLSQLGGDSGGSSGGSKRT |
| | ADGSEFESPKKKRKVSGGSSGGSSKRKAPQETLNGGITDMLTELANFEKNVS |
| | QAIHKYNAYRKAASVIAKYPHKIKSGAEAKKLPGVGTKIAEKIDEFLATGKL |
| | RKLEKIRQDDTSSSINFLTRVSGIGPSAARKFVDEGIKTLEDLRKNEDKLNHH |
| | QRIGLKYFGDFEKRIPREEMLQMQDIVLNEVKKVDSEYIATVCGSFRRGAESS |
| | GDMDVLLTHPSFTSESTKQPKLLHQVVEQLQKVHFITDTLSKGETKFMGVCQ |
| | LPSKNDEKEYPHRRIDIRLIPKDQYYCGVLYFTGSDIFNKNMRAHALEKGFTI |
| | NEYTIRPLGVTGVAGEPLPVDSEKDIFDYIQWKYREPKDRSEGGGSGGGSAT |
| | VKFKYKGEEKEVDISKIKKVWRVGKMISFTYDEGGGKTGRGAVSEKDAPKE |
| | LLQMLEKQKKSGGSKRTADGSEFESPKKKRKV (SEQ ID NO: 136) |
| PCV2- | MSPSKKNGRSGPQPHKRWVFTLNNPSEDERKKIRDLPISLFDYFIVGEEGNEE |
| XTEN- | GRTPHLQGFANFVKKQTFNKVKWYLGARCHIEKAKGTDQQNKEYCSKEGN |
| nCas9 | LLMECGAPRSQGQRSGGSSGSETPGTSESATPESSGGSDKKYSIGLDIGTNSV |
| (H840A)- | GWAVITDEYKVPSKKFKVLGNTDRHSIKKNLIGALLFDSGETAEATRLKRTA |
| SGGS | RRRYTRRKNRICYLQEIFSNEMAKVDDSFFHRLEESFLVEEDKKHERHPIFGN |
| (SEQ ID | IVDEVAYHEKYPTIYHLRKKLVDSTDKADLRLIYLALAHMIKFRGHFLIEGDL |
| NO: | NPDNSDVDKLFIQLVQTYNQLFEENPINASGVDAKAILSARLSKSRRLENLIA |
| 573)_ | QLPGEKKNGLFGNLIALSLGLTPNFKSNFDLAEDAKLQLSKDTYDDDLDNLL |
| NLS_ | AQIGDQYADLFLAAKNLSDAILLSDILRVNTEITKAPLSASMIKRYDEHHQDL |
| SGGS | TLLKALVRQQLPEKYKEIFFDQSKNGYAGYIDGGASQEEFYKFIKPILEKMDG |
| (SEQ ID | TEELLVKLNREDLLRKQRTFDNGSIPHQIHLGELHAILRRQEDFYPFLKDNRE |
| NO: | KIEKILTFRIPYYVGPLARGNSRFAWMTRKSEETITPWNFEEVVDKGASAQSF |
| 573)_- | IERMTNFDKNLPNEKVLPKHSLLYEYFTVYNELTKVKYVTEGMRKPAFLSGE |
| Sequenase- | QKKAIVDLLFKTNRKVTVKQLKEDYFKKIECFDSVEISGVEDRFNASLGTYH |
| NLS | DLLKIIKDKDFLDNEENEDILEDIVLTLTLFEDREMIEERLKTYAHLFDDKVM |
| | KQLKRRRYTGWGRLSRKLINGIRDKQSGKTILDFLKSDGFANRNFMQLIHDD |
| | SLTFKEDIQKAQVSGQGDSLHEHIANLAGSPAIKKGILQTVKVVDELVKVMG |
| | RHKPENIVIEMARENQTTQKGQKNSRERMKRIEEGIKELGSQILKEHPVENTQ |
| | LQNEKLYLYYLQNGRDMYVDQELDINRLSDYDVDAIVPQSFLKDDSIDNKV |
| | LTRSDKNRGKSDNVPSEEVVKKMKNYWRQLLNAKLITQRKFDNLTKAERG |
| | GLSELDKAGFIKRQLVETRQITKHVAQILDSRMNTKYDENDKLIREVKVITLK |
| | SKLVSDFRKDFQFYKVREINNYHHAHDAYLNAVVGTALIKKYPKLESEFVY |
| | GDYKVYDVRKMIAKSEQEIGKATAKYFFYSNIMNFFKTEITLANGEIRKRPLI |
| | ETNGETGEIVWDKGRDFATVRKVLSMPQVNIVKKTEVQTGGFSKESILPKRN |
| | SDKLIARKKDWDPKKYGGFDSPTVAYSVLVVAKVEKGKSKKLKSVKELLGI |
| | TIMERSSFEKNPIDFLEAKGYKEVKKDLIIKLPKYSLFELENGRKRMLASAGE |
| | LQKGNELALPSKYVNFLYLASHYEKLKGSPEDNEQKQLFVEQHKHYLDEIIE |
| | QISEFSKRVILADANLDKVLSAYNKHRDKPIREQAENIIHLFTLTNLGAPAAFK |
| | YFDTTIDRKRYTSTKEVLDATLIHQSITGLYETRIDLSQLGGDSGGSSGGSKRT |
| | ADGSEFESPKKKRKVSGGSSGGSIVSDIEANALLESVTKFHCGVIYDYSTAEY |
| | VSYRPSDFGAYLDALEAEVARGGLIVFHNGHKYDVPALTKLAKLQLNREFH |
| | LPRENCIDTLVLSRLIHSNLKDTDMGLLRSGKLPGMLEEQGEEYVDGMEWW |
| | NFNEEMMDYNVQDVVVTKALLEKLLSDKHYFPPEIDFTDVGYTTFWSESLE |
| | AVDIEHRAAWLLAKQERNGFPFDTKAIEELYVELAARRSELLRKLTETFGSW |
| | YQPKGGTEMFCHPRTGKPLPKYPRIKTPKVGGIFKKPKNKAQREGREPCELD |
| | TREYVAGAPYTPVEHVVFNPSSRDHIQKKLQEAGWVPTKYTDKGAPVVDDE |
| | VLEGVRVDDPEKQAAIDLIKEYLMIQKRIGQSAEGDKAWLRYVAEDGKIHGS |
| | VNPNGAVTGRATHAFPNLAQIPGVRSPYGEQCRAAFGAEHHLDGITGKPWV |
| | QAGIDASGLELRCLAHFMARFDNGEYAHEILNGDIHTKNQIAAELPTRDNAK |
| | TFIYGFLYGAGDEKIGQIVGAGKERGKELKKKFLENTPAIAALRESIQQTLVE |
| | SSQWVAGEQQVKWKRRWIKGLDGRKVHVRSPHAALNTLLQSAGALICKLW |
| | IIKTEEMLVEKGLKHGWDGDFAYMAWVHDEIQVGCRTEEIAQVVIETAQEA |
| | MRWVGDHWNFRCLLDTEGKMGPNWAICHSGGSKRTADGSEFESPKKKRKV |
| | (SEQ ID NO: 137) |
| PCV2- | MSPSKKNGRSGPQPHKRWVFTLNNPSEDERKKIRDLPISLFDYFIVGEEGNEE |
| XTEN24- | GRTPHLQGFANFVKKQTFNKVKWYLGARCHIEKAKGTDQQNKEYCSKEGN |
| nCas9 | LLMECGAPRSQGQRSGGSSGSETPGTSESATPESSGGSDKKYSIGLDIGTNSV |
| (H840A)- | GWAVITDEYKVPSKKFKVLGNTDRHSIKKNLIGALLFDSGETAEATRLKRTA |
| SGGS | RRRYTRRKNRICYLQEIFSNEMAKVDDSFFHRLEESFLVEEDKKHERHPIFGN |
| (SEQ ID | IVDEVAYHEKYPTIYHLRKKLVDSTDKADLRLIYLALAHMIKFRGHFLIEGDL |
| NO: | NPDNSDVDKLFIQLVQTYNQLFEENPINASGVDAKAILSARLSKSRRLENLIA |
| 573)_ | QLPGEKKNGLFGNLIALSLGLTPNFKSNFDLAEDAKLQLSKDTYDDDLDNLL |
| NLS_ | AQIGDQYADLFLAAKNLSDAILLSDILRVNTEITKAPLSASMIKRYDEHHQDL |
| SGGS | TLLKALVRQQLPEKYKEIFFDQSKNGYAGYIDGGASQEEFYKFIKPILEKMDG |
| (SEQ ID | TEELLVKLNREDLLRKQRTFDNGSIPHQIHLGELHAILRRQEDFYPFLKDNRE |
| NO: | KIEKILTFRIPYYVGPLARGNSRFAWMTRKSEETITPWNFEEVVDKGASAQSF |
| 573)- | IERMTNFDKNLPNEKVLPKHSLLYEYFTVYNELTKVKYVTEGMRKPAFLSGE |
| Phi29 | QKKAIVDLLFKTNRKVTVKQLKEDYFKKIECFDSVEISGVEDRFNASLGTYH |
| (D169A)- | DLLKIIKDKDFLDNEENEDILEDIVLTLTLFEDREMIEERLKTYAHLFDDKVM |
| NLS | KQLKRRRYTGWGRLSRKLINGIRDKQSGKTILDFLKSDGFANRNFMQLIHDD |
| | SLTFKEDIQKAQVSGQGDSLHEHIANLAGSPAIKKGILQTVKVVDELVKVMG |
| | RHKPENIVIEMARENQTTQKGQKNSRERMKRIEEGIKELGSQILKEHPVENTQ |
| | LQNEKLYLYYLQNGRDMYVDQELDINRLSDYDVDAIVPQSFLKDDSIDNKV |
| | LTRSDKNRGKSDNVPSEEVVKKMKNYWRQLLNAKLITQRKFDNLTKAERG |
| | GLSELDKAGFIKRQLVETRQITKHVAQILDSRMNTKYDENDKLIREVKVITLK |
| | SKLVSDFRKDFQFYKVREINNYHHAHDAYLNAVVGTALIKKYPKLESEFVY |
| | GDYKVYDVRKMIAKSEQEIGKATAKYFFYSNIMNFFKTEITLANGEIRKRPLI |
| | ETNGETGEIVWDKGRDFATVRKVLSMPQVNIVKKTEVQTGGFSKESILPKRN |
| | SDKLIARKKDWDPKKYGGFDSPTVAYSVLVVAKVEKGKSKKLKSVKELLGI |
| | TIMERSSFEKNPIDFLEAKGYKEVKKDLIIKLPKYSLFELENGRKRMLASAGE |
| | LQKGNELALPSKYVNFLYLASHYEKLKGSPEDNEQKQLFVEQHKHYLDEDIE |
| | QISEFSKRVILADANLDKVLSAYNKHRDKPIREQAENIIHLFTLTNLGAPAAFK |
| | YFDTTIDRKRYTSTKEVLDATLIHQSITGLYETRIDLSQLGGDSGGSSGGSKRT |
| | ADGSEFESPKKKRKVSGGSSGGSKHMPRKMYSCDFETTTKVEDCRVWAYG |
| | YMNIEDHSEYKIGNSLDEFMAWVLKVQADLYFHNLKFDGAFIINWLERNGF |
| | KWSADGLPNTYNTIISRMGQWYMIDICLGYKGKRKIHTVIYDSLKKLPFPVK |
| | KIAKDFKLTVLKGDIDYHKERPVGYKITPEEYAYIKNAIQIIAEALLIQFKQGL |
| | DRMTAGSDSLKGFKDIITTKKFKKVFPTLSLGLDKEVRYAYRGGFTWLNDRF |
| | KEKEIGEGMVFDVNSLYPAQMYSRLLPYGEPIVFEGKYVWDEDYPLHIQHIR |
| | CEFELKEGYIPTIQIKRSRFYKGNEYLKSSGGEIADLWLSNVDLELMKEHYDL |
| | YNVEYISGLKFKATTGLFKDFIDKWTYIKTTSEGAIKQLAKLMLNSLYGKFAS |
| | NPDVTGKVPYLKENGALGFRLGEEETKDPVYTPMGVFITAWARYTTITAAQ |
| | ACYDRIIYCDTDSIHLTGTEIPDVIKDIVDPKKLGYWAHESTFKRAKYLRQKT |
| | YIQDIYMKEVDGKLVEGSPDDYTDIKFSVKCAGMTDKIKKEVTFENFKVGFS |
| | RKMKPKPVQVPGGVVLVDDTFTIKSGGSKRTADGSEFESPKKKRKV (SEQ ID |
| | NO: 138) |
| dPCV2 | MSPSKKNGRSGPQPHKRWVFTLNNPSEDERKKIRDLPISLFDYFIVGEEGNEE |
| (Y96F)- | GRTPHLQGFANFVKKQTFNKVKWYLGARCHIEKAKGTDQQNKEFCSKEGN |
| XTEN- | LLMECGAPRSQGQRSGGSSGSETPGTSESATPESSGGSDKKYSIGLDIGTNSV |
| nCas9 | GWAVITDEYKVPSKKFKVLGNTDRHSIKKNLIGALLFDSGETAEATRLKRTA |
| (H840A)- | RRRYTRRKNRICYLQEIFSNEMAKVDDSFFHRLEESFLVEEDKKHERHPIFGN |
| SGGS | IVDEVAYHEKYPTIYHLRKKLVDSTDKADLRLIYLALAHMIKFRGHFLIEGDL |
| (SEQ ID | NPDNSDVDKLFIQLVQTYNQLFEENPINASGVDAKAILSARLSKSRRLENLIA |
| NO: | QLPGEKKNGLFGNLIALSLGLTPNFKSNFDLAEDAKLQLSKDTYDDDLDNLL |
| 573)_ | AQIGDQYADLFLAAKNLSDAILLSDILRVNTEITKAPLSASMIKRYDEHHQDL |
| NLS_ | TLLKALVRQQLPEKYKEIFFDQSKNGYAGYIDGGASQEEFYKFIKPILEKMDG |
| SGGS | TEELLVKLNREDLLRKQRTFDNGSIPHQIHLGELHAILRRQEDFYPFLKDNRE |
| (SEQ ID | KIEKILTFRIPYYVGPLARGNSRFAWMTRKSEETITPWNFEEVVDKGASAQSF |
| NO: | IERMTNFDKNLPNEKVLPKHSLLYEYFTVYNELTKVKYVTEGMRKPAFLSGE |
| 573)- | QKKAIVDLLFKTNRKVTVKQLKEDYFKKIECFDSVEISGVEDRFNASLGTYH |
| EcKLenow | DLLKIIKDKDFLDNEENEDILEDIVLTLTLFEDREMIEERLKTYAHLFDDKVM |
| (D355A, | KQLKRRRYTGWGRLSRKLINGIRDKQSGKTILDFLKSDGFANRNFMQLIHDD |
| D357A)-NLS | SLTFKEDIQKAQVSGQGDSLHEHIANLAGSPAIKKGILQTVKVVDELVKVMG |
| | RHKPENIVIEMARENQTTQKGQKNSRERMKRIEEGIKELGSQILKEHPVENTQ |
| | LQNEKLYLYYLQNGRDMYVDQELDINRLSDYDVDAIVPQSFLKDDSIDNKV |
| | LTRSDKNRGKSDNVPSEEVVKKMKNYWRQLLNAKLITQRKFDNLTKAERG |
| | GLSELDKAGFIKRQLVETRQITKHVAQILDSRMNTKYDENDKLIREVKVITLK |
| | SKLVSDFRKDFQFYKVREINNYHHAHDAYLNAVVGTALIKKYPKLESEFVY |
| | GDYKVYDVRKMIAKSEQEIGKATAKYFFYSNIMNFFKTEITLANGEIRKRPLI |
| | ETNGETGEIVWDKGRDFATVRKVLSMPQVNIVKKTEVQTGGFSKESILPKRN |
| | SDKLIARKKDWDPKKYGGFDSPTVAYSVLVVAKVEKGKSKKLKSVKELLGI |
| | TIMERSSFEKNPIDFLEAKGYKEVKKDLIIKLPKYSLFELENGRKRMLASAGE |
| | LQKGNELALPSKYVNFLYLASHYEKLKGSPEDNEQKQLFVEQHKHYLDEIIE |
| | QISEFSKRVILADANLDKVLSAYNKHRDKPIREQAENIIHLFTLTNLGAPAAFK |
| | YFDTTIDRKRYTSTKEVLDATLIHQSITGLYETRIDLSQLGGDSGGSSGGSKRT |
| | ADGSEFESPKKKRKVSGGSSGGSVISYDNYVTILDEETLKAWIAKLEKAPVFA |
| | FATATDSLDNISANLVGLSFAIEPGVAAYIPVAHDYLDAPDQISRERALELLKP |
| | LLEDEKALKVGQNLKYDRGILANYGIELRGIAFDTMLESYILNSVAGRHDMD |
| | SLAERWLKHKTITFEEIAGKGKNQLTFNQIALEEAGRYAAEDADVTLQLHLK |
| | MWPDLQKHKGPLNVFENIEMPLVPVLSRIERNGVKIDPKVLHNHSEELTLRL |
| | AELEKKAHEIAGEEFNLSSTKQLQTILFEKQGIKPLKKTPGGAPSTSEEVLEEL |
| | ALDYPLPKVILEYRGLAKLKSTYTDKLPLMINPKTGRVHTSYHQAVTATGRL |
| | SSTDPNLQNIPVRNEEGRRIRQAFIAPEDYVIVSADYSQIELRIMAHLSRDKGL |
| | LTAFAEGKDIHRATAAEVFGLPLETVTSEQRRSAKAINFGLIYGMSAFGLARQ |
| | LNIPRKEAQKYMDLYFERYPGVLEYMERTRAQAKEQGYVETLDGRRLYLPD |
| | IKSSNGARRAAAERAAINAPMQGTAADIIKRAMIAVDAWLQAEQPRVRMIM |
| | QVHDELVFEVHKDDVDAVAKQIHQLMENCTRLDVPLLVEVGSGENWDQAH |
| | SGGSKRTADGSEFESPKKKRKV (SEQ ID NO: 139) |
| DCV- | MAKSGNYSYKRWVFTINNPTFEDYVHVLEFCTLDNCKFAIVGEEKGANGTP |
| XTEN- | HLQGFLNLRSNARAAALEESLGGRAWLSRARGSDEDNEEYCAKESTYLRVG |
| nCas9 | EPVSKGRSSDLAEATSAVSGGSSGSETPGTSESATPESSGGSDKKYSIGLDIGT |
| (H840A)- | NSVGWAVITDEYKVPSKKFKVLGNTDRHSIKKNLIGALLFDSGETAEATRLK |
| SGGS | RTARRRYTRRKNRICYLQEIFSNEMAKVDDSFFHRLEESFLVEEDKKHERHPI |
| (SEQ ID | FGNIVDEVAYHEKYPTIYHLRKKLVDSTDKADLRLIYLALAHMIKFRGHFLIE |
| NO: | GDLNPDNSDVDKLFIQLVQTYNQLFEENPINASGVDAKAILSARLSKSRRLEN |
| 573)_ | LIAQLPGEKKNGLFGNLIALSLGLTPNFKSNFDLAEDAKLQLSKDTYDDDLD |
| NLS_ | NLLAQIGDQYADLFLAAKNLSDAILLSDILRVNTEITKAPLSASMIKRYDEHH |
| SGGS | QDLTLLKALVRQQLPEKYKEIFFDQSKNGYAGYIDGGASQEEFYKFIKPILEK |
| (SEQ ID | MDGTEELLVKLNREDLLRKQRTFDNGSIPHQIHLGELHAILRRQEDFYPFLKD |
| NO: | NREKIEKILTFRIPYYVGPLARGNSRFAWMTRKSEETITPWNFEEVVDKGASA |
| 573)- | QSFIERMTNFDKNLPNEKVLPKHSLLYEYFTVYNELTKVKYVTEGMRKPAFL |
| EcKLenow | SGEQKKAIVDLLFKTNRKVTVKQLKEDYFKKIECFDSVEISGVEDRFNASLGT |
| (D355A, | YHDLLKIIKDKDFLDNEENEDILEDIVLTLTLFEDREMIEERLKTYAHLFDDK |
| D357A)- | VMKQLKRRRYTGWGRLSRKLINGIRDKQSGKTILDFLKSDGFANRNFMQLIH |
| NLS | DDSLTFKEDIQKAQVSGQGDSLHEHIANLAGSPAIKKGILQTVKVVDELVKV |
| | MGRHKPENIVIEMARENQTTQKGQKNSRERMKRIEEGIKELGSQILKEHPVE |
| | NTQLQNEKLYLYYLQNGRDMYVDQELDINRLSDYDVDAIVPQSFLKDDSID |
| | NKVLTRSDKNRGKSDNVPSEEVVKKMKNYWRQLLNAKLITQRKFDNLTKA |
| | ERGGLSELDKAGFIKRQLVETRQITKHVAQILDSRMNTKYDENDKLIREVKVI |
| | TLKSKLVSDFRKDFQFYKVREINNYHHAHDAYLNAVVGTALIKKYPKLESEF |
| | VYGDYKVYDVRKMIAKSEQEIGKATAKYFFYSNIMNFFKTEITLANGEIRKR |
| | PLIETNGETGEIVWDKGRDFATVRKVLSMPQVNIVKKTEVQTGGFSKESILPK |
| | RNSDKLIARKKDWDPKKYGGFDSPTVAYSVLVVAKVEKGKSKKLKSVKELL |
| | GITIMERSSFEKNPIDFLEAKGYKEVKKDLIIKLPKYSLFELENGRKRMLASAG |
| | ELQKGNELALPSKYVNFLYLASHYEKLKGSPEDNEQKQLFVEQHKHYLDEII |
| | EQISEFSKRVILADANLDKVLSAYNKHRDKPIREQAENIIHLFTLTNLGAPAAF |
| | KYFDTTIDRKRYTSTKEVLDATLIHQSITGLYETRIDLSQLGGDSGGSSGGSKR |
| | TADGSEFESPKKKRKVSGGSSGGSVISYDNYVTILDEETLKAWIAKLEKAPVF |
| | AFATATDSLDNISANLVGLSFAIEPGVAAYIPVAHDYLDAPDQISRERALELL |
| | KPLLEDEKALKVGQNLKYDRGILANYGIELRGIAFDTMLESYILNSVAGRHD |
| | MDSLAERWLKHKTITFEEIAGKGKNQLTFNQIALEEAGRYAAEDADVTLQL |
| | HLKMWPDLQKHKGPLNVFENIEMPLVPVLSRIERNGVKIDPKVLHNHSEELT |
| | LRLAELEKKAHEIAGEEFNLSSTKQLQTILFEKQGIKPLKKTPGGAPSTSEEVL |
| | EELALDYPLPKVILEYRGLAKLKSTYTDKLPLMINPKTGRVHTSYHQAVTAT |
| | GRLSSTDPNLQNIPVRNEEGRRIRQAFIAPEDYVIVSADYSQIELRIMAHLSRD |
| | KGLLTAFAEGKDIHRATAAEVFGLPLETVTSEQRRSAKAINFGLIYGMSAFGL |
| | ARQLNIPRKEAQKYMDLYFERYPGVLEYMERTRAQAKEQGYVETLDGRRL |
| | YLPDIKSSNGARRAAAERAAINAPMQGTAADIIKRAMIAVDAWLQAEQPRV |
| | RMIMQVHDELVFEVHKDDVDAVAKQIHQLMENCTRLDVPLLVEVGSGENW |
| | DQAHSGGSKRTADGSEFESPKKKRKV (SEQ ID NO: 140) |
| FBNYV- | MARQVICWCFTLNNPLSPLSLHDSMKYLVYQTEQGEAGNIHFQGYIEMKKR |
| XTEN- | TSLAGMKKLIPGAHFEKRRGTQGEARAYSMKEDTRLEGPWEYGEFVPSGGS |
| nCas9 | SGSETPGTSESATPESSGGSDKKYSIGLDIGTNSVGWAVITDEYKVPSKKFKV |
| (H840A)- | LGNTDRHSIKKNLIGALLFDSGETAEATRLKRTARRRYTRRKNRICYLQEIFS |
| SGGS | NEMAKVDDSFFHRLEESFLVEEDKKHERHPIFGNIVDEVAYHEKYPTIYHLR |
| (SEQ ID | KKLVDSTDKADLRLIYLALAHMIKFRGHFLIEGDLNPDNSDVDKLFIQLVQT |
| NO: | YNQLFEENPINASGVDAKAILSARLSKSRRLENLIAQLPGEKKNGLFGNLIALS |
| 573)_ | LGLTPNFKSNFDLAEDAKLQLSKDTYDDDLDNLLAQIGDQYADLFLAAKNL |
| NLS_ | SDAILLSDILRVNTEITKAPLSASMIKRYDEHHQDLTLLKALVRQQLPEKYKEI |
| SGGS | FFDQSKNGYAGYIDGGASQEEFYKFIKPILEKMDGTEELLVKLNREDLLRKQ |
| (SEQ ID | RTFDNGSIPHQIHLGELHAILRRQEDFYPFLKDNREKIEKILTFRIPYYVGPLAR |
| NO: | GNSRFAWMTRKSEETITPWNFEEVVDKGASAQSFIERMTNFDKNLPNEKVLP |
| 573)- | KHSLLYEYFTVYNELTKVKYVTEGMRKPAFLSGEQKKAIVDLLFKTNRKVT |
| EcKLenow | VKQLKEDYFKKIECFDSVEISGVEDRFNASLGTYHDLLKIIKDKDFLDNEENE |
| (D355A, | DILEDIVLTLTLFEDREMIEERLKTYAHLFDDKVMKQLKRRRYTGWGRLSRK |
| D357A)- | LINGIRDKQSGKTILDFLKSDGFANRNFMQLIHDDSLTFKEDIQKAQVSGQGD |
| NLS | SLHEHIANLAGSPAIKKGILQTVKVVDELVKVMGRHKPENIVIEMARENQTT |
| | QKGQKNSRERMKRIEEGIKELGSQILKEHPVENTQLQNEKLYLYYLQNGRD |
| | MYVDQELDINRLSDYDVDAIVPQSFLKDDSIDNKVLTRSDKNRGKSDNVPSE |
| | EVVKKMKNYWRQLLNAKLITQRKFDNLTKAERGGLSELDKAGFIKRQLVET |
| | RQITKHVAQILDSRMNTKYDENDKLIREVKVITLKSKLVSDFRKDFQFYKVR |
| | EINNYHHAHDAYLNAVVGTALIKKYPKLESEFVYGDYKVYDVRKMIAKSEQ |
| | EIGKATAKYFFYSNIMNFFKTEITLANGEIRKRPLIETNGETGEIVWDKGRDFA |
| | TVRKVLSMPQVNIVKKTEVQTGGFSKESILPKRNSDKLIARKKDWDPKKYG |
| | GFDSPTVAYSVLVVAKVEKGKSKKLKSVKELLGITIMERSSFEKNPIDFLEAK |
| | GYKEVKKDLIIKLPKYSLFELENGRKRMLASAGELQKGNELALPSKYVNFLY |
| | LASHYEKLKGSPEDNEQKQLFVEQHKHYLDEIIEQISEFSKRVILADANLDKV |
| | LSAYNKHRDKPIREQAENIIHLFTLTNLGAPAAFKYFDTTIDRKRYTSTKEVL |
| | DATLIHQSITGLYETRIDLSQLGGDSGGSSGGSKRTADGSEFESPKKKRKVSG |
| | GSSGGSVISYDNYVTILDEETLKAWIAKLEKAPVFAFATATDSLDNISANLVG |
| | LSFAIEPGVAAYIPVAHDYLDAPDQISRERALELLKPLLEDEKALKVGQNLKY |
| | DRGILANYGIELRGIAFDTMLESYILNSVAGRHDMDSLAERWLKHKTITFEEI |
| | AGKGKNQLTFNQIALEEAGRYAAEDADVTLQLHLKMWPDLQKHKGPLNVF |
| | ENIEMPLVPVLSRIERNGVKIDPKVLHNHSEELTLRLAELEKKAHEIAGEEFNL |
| | SSTKQLQTILFEKQGIKPLKKTPGGAPSTSEEVLEELALDYPLPKVILEYRGLA |
| | KLKSTYTDKLPLMINPKTGRVHTSYHQAVTATGRLSSTDPNLQNIPVRNEEG |
| | RRIRQAFIAPEDYVIVSADYSQIELRIMAHLSRDKGLLTAFAEGKDIHRATAA |
| | EVFGLPLETVTSEQRRSAKAINFGLIYGMSAFGLARQLNIPRKEAQKYMDLY |
| | FERYPGVLEYMERTRAQAKEQGYVETLDGRRLYLPDIKSSNGARRAAAERA |
| | AINAPMQGTAADIIKRAMIAVDAWLQAEQPRVRMIMQVHDELVFEVHKDD |
| | VDAVAKQIHQLMENCTRLDVPLLVEVGSGENWDQAHSGGSKRTADGSEFES |
| | PKKKRKV (SEQ ID NO: 141) |
| RepBm- | MSEKKEIVKGRDWTFLVYPESAPENWRTILDETFMRWVESPLHDKDVNADG |
| XTEN- | EIKKPHWHILLSSDGPITQTAVQKIIGPLNAPNAQKVGSAKGLVRYMVHLDN |
| nCas9 | PEKYQYSLDEIVGHNGADVASYFELTASGGSSGSETPGTSESATPESSGGSDK |
| (H840A)- | KYSIGLDIGTNSVGWAVITDEYKVPSKKFKVLGNTDRHSIKKNLIGALLFDSG |
| SGGS | ETAEATRLKRTARRRYTRRKNRICYLQEIFSNEMAKVDDSFFHRLEESFLVEE |
| (SEQ ID | DKKHERHPIFGNIVDEVAYHEKYPTIYHLRKKLVDSTDKADLRLIYLALAHM |
| NO: | IKFRGHFLIEGDLNPDNSDVDKLFIQLVQTYNQLFEENPINASGVDAKAILSAR |
| 573)_ | LSKSRRLENLIAQLPGEKKNGLFGNLIALSLGLTPNFKSNFDLAEDAKLQLSK |
| NLS_ | DTYDDDLDNLLAQIGDQYADLFLAAKNLSDAILLSDILRVNTEITKAPLSASM |
| SGGS | IKRYDEHHQDLTLLKALVRQQLPEKYKEIFFDQSKNGYAGYIDGGASQEEFY |
| (SEQ ID | KFIKPILEKMDGTEELLVKLNREDLLRKQRTFDNGSIPHQIHLGELHAILRRQE |
| NO: | DFYPFLKDNREKIEKILTFRIPYYVGPLARGNSRFAWMTRKSEETITPWNFEE |
| 573)- | VVDKGASAQSFIERMTNFDKNLPNEKVLPKHSLLYEYFTVYNELTKVKYVT |
| EcKLenow | EGMRKPAFLSGEQKKAIVDLLFKTNRKVTVKQLKEDYFKKIECFDSVEISGV |
| (D355A, | EDRFNASLGTYHDLLKIIKDKDFLDNEENEDILEDIVLTLTLFEDREMIEERLK |
| D357A)- | TYAHLFDDKVMKQLKRRRYTGWGRLSRKLINGIRDKQSGKTILDFLKSDGF |
| NLS | ANRNFMQLIHDDSLTFKEDIQKAQVSGQGDSLHEHIANLAGSPAIKKGILQTV |
| | KVVDELVKVMGRHKPENIVIEMARENQTTQKGQKNSRERMKRIEEGIKELG |
| | SQILKEHPVENTQLQNEKLYLYYLQNGRDMYVDQELDINRLSDYDVDAIVP |
| | QSFLKDDSIDNKVLTRSDKNRGKSDNVPSEEVVKKMKNYWRQLLNAKLITQ |
| | RKFDNLTKAERGGLSELDKAGFIKRQLVETRQITKHVAQILDSRMNTKYDEN |
| | DKLIREVKVITLKSKLVSDFRKDFQFYKVREINNYHHAHDAYLNAVVGTALI |
| | KKYPKLESEFVYGDYKVYDVRKMIAKSEQEIGKATAKYFFYSNIMNFFKTEI |
| | TLANGEIRKRPLIETNGETGEIVWDKGRDFATVRKVLSMPQVNIVKKTEVQT |
| | GGFSKESILPKRNSDKLIARKKDWDPKKYGGFDSPTVAYSVLVVAKVEKGK |
| | SKKLKSVKELLGITIMERSSFEKNPIDFLEAKGYKEVKKDLIIKLPKYSLFELE |
| | NGRKRMLASAGELQKGNELALPSKYVNFLYLASHYEKLKGSPEDNEQKQLF |
| | VEQHKHYLDEIIEQISEFSKRVILADANLDKVLSAYNKHRDKPIREQAENIIHL |
| | FTLTNLGAPAAFKYFDTTIDRKRYTSTKEVLDATLIHQSITGLYETRIDLSQLG |
| | GDSGGSSGGSKRTADGSEFESPKKKRKVSGGSSGGSVISYDNYVTILDEETLK |
| | AWIAKLEKAPVFAFATATDSLDNISANLVGLSFAIEPGVAAYIPVAHDYLDAP |
| | DQISRERALELLKPLLEDEKALKVGQNLKYDRGILANYGIELRGIAFDTMLES |
| | YILNSVAGRHDMDSLAERWLKHKTITFEEIAGKGKNQLTFNQIALEEAGRYA |
| | AEDADVTLQLHLKMWPDLQKHKGPLNVFENIEMPLVPVLSRIERNGVKIDPK |
| | VLHNHSEELTLRLAELEKKAHEIAGEEFNLSSTKQLQTILFEKQGIKPLKKTPG |
| | GAPSTSEEVLEELALDYPLPKVILEYRGLAKLKSTYTDKLPLMINPKTGRVHT |
| | SYHQAVTATGRLSSTDPNLQNIPVRNEEGRRIRQAFIAPEDYVIVSADYSQIEL |
| | RIMAHLSRDKGLLTAFAEGKDIHRATAAEVFGLPLETVTSEQRRSAKAINFGL |
| | IYGMSAFGLARQLNIPRKEAQKYMDLYFERYPGVLEYMERTRAQAKEQGYV |
| | ETLDGRRLYLPDIKSSNGARRAAAERAAINAPMQGTAADIIKRAMIAVDAWL |
| | QAEQPRVRMIMQVHDELVFEVHKDDVDAVAKQIHQLMENCTRLDVPLLVE |
| | VGSGENWDQAHSGGSKRTADGSEFESPKKKRKV (SEQ ID NO: 142) |
| TraI- | MMSIAQVRSAGSAGNYYTDKDNYYVLGSMGERWAGRGAEQLGLQGSVDK |
| XTEN- | DVFTRLLEGRLPDGADLSRMQDGSNRHRPGYDLTFSAPKSVSMMAMLGGD |
| nCas9 | KRLIDAHNQAVDFAVRQVEALASTRVMTDGQSETVLTGNLVMALFNHDTS |
| (H840A)- | RDQEPQLHTHAVVANVTQHNGEWKTLSSDKVGKTGFIENVYANQIAFGRLY |
| SGGS | REKLKEQVEALGYETEVVGKHGMWEMPGVPVEAFSGRSQTIREAVGEDASL |
| (SEQ ID | KSRDVAALDTRKSKQHVDPEIKMAEWMQTLKETGFDIRAYRDAADQRADL |
| NO: | RTLTPGPASQDGPDVQQAVTQAIAGLSERSGGSSGSETPGTSESATPESSGGS |
| 573)_ | DKKYSIGLDIGTNSVGWAVITDEYKVPSKKFKVLGNTDRHSIKKNLIGALLFD |
| NLS_ | SGETAEATRLKRTARRRYTRRKNRICYLQEIFSNEMAKVDDSFFHRLEESFLV |
| SGGS | EEDKKHERHPIFGNIVDEVAYHEKYPTIYHLRKKLVDSTDKADLRLIYLALA |
| (SEQ ID | HMIKFRGHFLIEGDLNPDNSDVDKLFIQLVQTYNQLFEENPINASGVDAKAIL |
| NO: | SARLSKSRRLENLIAQLPGEKKNGLFGNLIALSLGLTPNFKSNFDLAEDAKLQ |
| 573)- | LSKDTYDDDLDNLLAQIGDQYADLFLAAKNLSDAILLSDILRVNTEITKAPLS |
| EcKLenow | ASMIKRYDEHHQDLTLLKALVRQQLPEKYKEIFFDQSKNGYAGYIDGGASQE |
| (D355A, | EFYKFIKPILEKMDGTEELLVKLNREDLLRKQRTFDNGSIPHQIHLGELHAILR |
| D357A)- | RQEDFYPFLKDNREKIEKILTFRIPYYVGPLARGNSRFAWMTRKSEETITPWN |
| NLS | FEEVVDKGASAQSFIERMTNFDKNLPNEKVLPKHSLLYEYFTVYNELTKVKY |
| | VTEGMRKPAFLSGEQKKAIVDLLFKTNRKVTVKQLKEDYFKKIECFDSVEIS |
| | GVEDRFNASLGTYHDLLKIIKDKDFLDNEENEDILEDIVLTLTLFEDREMIEER |
| | LKTYAHLFDDKVMKQLKRRRYTGWGRLSRKLINGIRDKQSGKTILDFLKSD |
| | GFANRNFMQLIHDDSLTFKEDIQKAQVSGQGDSLHEHIANLAGSPAIKKGILQ |
| | TVKVVDELVKVMGRHKPENIVIEMARENQTTQKGQKNSRERMKRIEEGIKE |
| | LGSQILKEHPVENTQLQNEKLYLYYLQNGRDMYVDQELDINRLSDYDVDAI |
| | VPQSFLKDDSIDNKVLTRSDKNRGKSDNVPSEEVVKKMKNYWRQLLNAKLI |
| | TQRKFDNLTKAERGGLSELDKAGFIKRQLVETRQITKHVAQILDSRMNTKYD |
| | ENDKLIREVKVITLKSKLVSDFRKDFQFYKVREINNYHHAHDAYLNAVVGT |
| | ALIKKYPKLESEFVYGDYKVYDVRKMIAKSEQEIGKATAKYFFYSNIMNFFK |
| | TEITLANGEIRKRPLIETNGETGEIVWDKGRDFATVRKVLSMPQVNIVKKTEV |
| | QTGGFSKESILPKRNSDKLIARKKDWDPKKYGGFDSPTVAYSVLVVAKVEK |
| | GKSKKLKSVKELLGITIMERSSFEKNPIDFLEAKGYKEVKKDLIIKLPKYSLFE |
| | LENGRKRMLASAGELQKGNELALPSKYVNFLYLASHYEKLKGSPEDNEQKQ |
| | LFVEQHKHYLDEIIEQISEFSKRVILADANLDKVLSAYNKHRDKPIREQAENII |
| | HLFTLTNLGAPAAFKYFDTTIDRKRYTSTKEVLDATLIHQSITGLYETRIDLSQ |
| | LGGDSGGSSGGSKRTADGSEFESPKKKRKVSGGSSGGSVISYDNYVTILDEET |
| | LKAWIAKLEKAPVFAFATATDSLDNISANLVGLSFAIEPGVAAYIPVAHDYLD |
| | APDQISRERALELLKPLLEDEKALKVGQNLKYDRGILANYGIELRGIAFDTML |
| | ESYILNSVAGRHDMDSLAERWLKHKTITFEEIAGKGKNQLTFNQIALEEAGR |
| | YAAEDADVTLQLHLKMWPDLQKHKGPLNVFENIEMPLVPVLSRIERNGVKI |
| | DPKVLHNHSEELTLRLAELEKKAHEIAGEEFNLSSTKQLQTILFEKQGIKPLK |
| | KTPGGAPSTSEEVLEELALDYPLPKVILEYRGLAKLKSTYTDKLPLMINPKTG |
| | RVHTSYHQAVTATGRLSSTDPNLQNIPVRNEEGRRIRQAFIAPEDYVIVSADY |
| | SQIELRIMAHLSRDKGLLTAFAEGKDIHRATAAEVFGLPLETVTSEQRRSAKA |
| | INFGLIYGMSAFGLARQLNIPRKEAQKYMDLYFERYPGVLEYMERTRAQAK |
| | EQGYVETLDGRRLYLPDIKSSNGARRAAAERAAINAPMQGTAADIIKRAMIA |
| | VDAWLQAEQPRVRMIMQVHDELVFEVHKDDVDAVAKQIHQLMENCTRLD |
| | VPLLVEVGSGENWDQAHSGGSKRTADGSEFESPKKKRKV (SEQ ID NO: 143) |
| MSMV- | MSHTSFRFRAKNVFLTYPRCPIGPEFLCDHLWNLVTPYDPLYVHVAQENHKDGGLHSHVLIQT |
| XTEN- | RIEISTFDPTYFDYTGTSIPGAVVFHPNIQACRNVRDCLAYIRKNTINEVSKGASGGSSGSETPGT |
| nCas9 | SESATPESSGGSDKKYSIGLDIGTNSVGWAVITDEYKVPSKKFKVLGNTDRHSIKKNLIGALLFDS |
| (H840A)- | GETAEATRLKRTARRRYTRRKNRICYLQEIFSNEMAKVDDSFFHRLEESFLVEEDKKHERHPIFG |
| SGGS | NIVDEVAYHEKYPTIYHLRKKLVDSTDKADLRLIYLALAHMIKFRGHFLIEGDLNPDNSDVDKLFI |
| (SEQ ID | QLVQTYNQLFEENPINASGVDAKAILSARLSKSRRLENLIAQLPGEKKNGLFGNLIALSLGLTPNF |
| NO: | KSNFDLAEDAKLQLSKDTYDDDLDNLLAQIGDQYADLFLAAKNLSDAILLSDILRVNTEITKAPLS |
| 573)_ | ASMIKRYDEHHQDLTLLKALVRQQLPEKYKEIFFDQSKNGYAGYIDGGASQEEFYKFIKPILEKM |
| NLS_SGGS | DGTEELLVKLNREDLLRKQRTFDNGSIPHQIHLGELHAILRRQEDFYPFLKDNREKIEKILTFRIPYY |
| (SEQ ID | VGPLARGNSRFAWMTRKSEETITPWNFEEVVDKGASAQSFIERMTNFDKNLPNEKVLPKHSLL |
| NO: | YEYFTVYNELTKVKYVTEGMRKPAFLSGEQKKAIVDLLFKTNRKVTVKQLKEDYFKKIECFDSVEI |
| 573)- | SGVEDRFNASLGTYHDLLKIIKDKDFLDNEENEDILEDIVLTLTLFEDREMIEERLKTYAHLFDDKV |
| EckLenow | MKQLKRRRYTGWGRLSRKLINGIRDKQSGKTILDFLKSDGFANRNFMQLIHDDSLTFKEDIQKA |
| (D355A, | QVSGQGDSLHEHIANLAGSPAIKKGILQTVKVVDELVKVMGRHKPENIVIEMARENQTTQKGQ |
| D357A)- | KNSRERMKRIEEGIKELGSQILKEHPVENTQLQNEKLYLYYLQNGRDMYVDQELDINRLSDYDV |
| NLS | DAIVPQSFLKDDSIDNKVLTRSDKNRGKSDNVPSEEVVKKMKNYWRQLLNAKLITQRKFDNLT |
| | KAERGGLSELDKAGFIKRQLVETRQITKHVAQILDSRMNTKYDENDKLIREVKVITLKSKLVSDFR |
| | KDFQFYKVREINNYHHAHDAYLNAVVGTALIKKYPKLESEFVYGDYKVYDVRKMIAKSEQEIGK |
| | ATAKYFFYSNIMNFFKTEITLANGEIRKRPLIETNGETGEIVWDKGRDFATVRKVLSMPQVNIVK |
| | KTEVQTGGFSKESILPKRNSDKLIARKKDWDPKKYGGFDSPTVAYSVLVVAKVEKGKSKKLKSVK |
| | ELLGITIMERSSFEKNPIDFLEAKGYKEVKKDLIIKLPKYSLFELENGRKRMLASAGELQKGNELAL |
| | PSKYVNFLYLASHYEKLKGSPEDNEQKQLFVEQHKHYLDEIIEQISEFSKRVILADANLDKVLSAY |
| | NKHRDKPIREQAENIIHLFTLTNLGAPAAFKYFDTTIDRKRYTSTKEVLDATLIHQSITGLYETRIDL |
| | SQLGGDSGGSSGGSKRTADGSEFESPKKKRKVSGGSSGGSVISYDNYVTILDEETLKAWIAKLEK |
| | APVFAFATATDSLDNISANLVGLSFAIEPGVAAYIPVAHDYLDAPDQISRERALELLKPLLEDEKAL |
| | KVGQNLKYDRGILANYGIELRGIAFDTMLESYILNSVAGRHDMDSLAERWLKHKTITFEEIAGKG |
| | KNQLTFNQIALEEAGRYAAEDADVTLQLHLKMWPDLQKHKGPLNVFENIEMPLVPVLSRIERN |
| | GVKIDPKVLHNHSEELTLRLAELEKKAHEIAGEEFNLSSTKQLQTILFEKQGIKPLKKTPGGAPSTS |
| | EEVLEELALDYPLPKVILEYRGLAKLKSTYTDKLPLMINPKTGRVHTSYHQAVTATGRLSSTDPNL |
| | QNIPVRNEEGRRIRQAFIAPEDYVIVSADYSQIELRIMAHLSRDKGLLTAFAEGKDIHRATAAEVF |
| | GLPLETVTSEQRRSAKAINFGLIYGMSAFGLARQLNIPRKEAQKYMDLYFERYPGVLEYMERTR |
| | AQAKEQGYVETLDGRRLYLPDIKSSNGARRAAAERAAINAPMQGTAADIIKRAMIAVDAWLQ |
| | AEQPRVRMIMQVHDELVFEVHKDDVDAVAKQIHQLMENCTRLDVPLLVEVGSGENWDQAH |
| | SGGSKRTADGSEFESPKKKRKV (SEQ ID NO: 144) |
| TGMV- | MPSHPKRFQINAKNYFLTYPQCSLSKEESLSQLQALNTPINKKFIKICRELHEDGQPHLHVLIQFE |
| XTEN- | GKYCCQNQRFFDLVSPTRSAHFHPNIQRAKSSSDVKTYIDKDGDTLVWGEFQVDGRSASGGSS |
| nCas9 | GSETPGTSESATPESSGGSDKKYSIGLDIGTNSVGWAVITDEYKVPSKKFKVLGNTDRHSIKKNLI |
| (H840A)- | GALLFDSGETAEATRLKRTARRRYTRRKNRICYLQEIFSNEMAKVDDSFFHRLEESFLVEEDKKHE |
| SGGS | RHPIFGNIVDEVAYHEKYPTIYHLRKKLVDSTDKADLRLIYLALAHMIKFRGHFLIEGDLNPDNSD |
| (SEQ ID | VDKLFIQLVQTYNQLFEENPINASGVDAKAILSARLSKSRRLENLIAQLPGEKKNGLFGNLIALSLG |
| NO: | LTPNFKSNFDLAEDAKLQLSKDTYDDDLDNLLAQIGDQYADLFLAAKNLSDAILLSDILRVNTEIT |
| 573)_ | KAPLSASMIKRYDEHHQDLTLLKALVRQQLPEKYKEIFFDQSKNGYAGYIDGGASQEEFYKFIKPI |
| NLS_SGGS | LEKMDGTEELLVKLNREDLLRKQRTFDNGSIPHQIHLGELHAILRRQEDFYPFLKDNREKIEKILTF |
| (SEQ ID | RIPYYVGPLARGNSRFAWMTRKSEETITPWNFEEVVDKGASAQSFIERMTNFDKNLPNEKVLP |
| NO: | KHSLLYEYFTVYNELTKVKYVTEGMRKPAFLSGEQKKAIVDLLFKTNRKVTVKQLKEDYFKKIECF |
| 573)- | DSVEISGVEDRFNASLGTYHDLLKIIKDKDFLDNEENEDILEDIVLTLTLFEDREMIEERLKTYAHLF |
| EckLenow | DDKVMKQLKRRRYTGWGRLSRKLINGIRDKQSGKTILDFLKSDGFANRNFMQLIHDDSLTFKE |
| (D355A, | DIQKAQVSGQGDSLHEHIANLAGSPAIKKGILQTVKVVDELVKVMGRHKPENIVIEMARENQT |
| D357A)- | TQKGQKNSRERMKRIEEGIKELGSQILKEHPVENTQLQNEKLYLYYLQNGRDMYVDQELDINRL |
| NLS | SDYDVDAIVPQSFLKDDSIDNKVLTRSDKNRGKSDNVPSEEVVKKMKNYWRQLLNAKLITQRK |
| | FDNLTKAERGGLSELDKAGFIKRQLVETRQITKHVAQILDSRMNTKYDENDKLIREVKVITLKSKL |
| | VSDFRKDFQFYKVREINNYHHAHDAYLNAVVGTALIKKYPKLESEFVYGDYKVYDVRKMIAKSE |
| | QEIGKATAKYFFYSNIMNFFKTEITLANGEIRKRPLIETNGETGEIVWDKGRDFATVRKVLSMPQ |
| | VNIVKKTEVQTGGFSKESILPKRNSDKLIARKKDWDPKKYGGFDSPTVAYSVLVVAKVEKGKSKK |
| | LKSVKELLGITIMERSSFEKNPIDFLEAKGYKEVKKDLIIKLPKYSLFELENGRKRMLASAGELQKG |
| | NELALPSKYVNFLYLASHYEKLKGSPEDNEQKQLFVEQHKHYLDEIIEQISEFSKRVILADANLDKV |
| | LSAYNKHRDKPIREQAENIIHLFTLTNLGAPAAFKYFDTTIDRKRYTSTKEVLDATLIHQSITGLYET |
| | RIDLSQLGGDSGGSSGGSKRTADGSEFESPKKKRKVSGGSSGGSVISYDNYVTILDEETLKAWIA |
| | KLEKAPVFAFATATDSLDNISANLVGLSFAIEPGVAAYIPVAHDYLDAPDQISRERALELLKPLLED |
| | EKALKVGQNLKYDRGILANYGIELRGIAFDTMLESYILNSVAGRHDMDSLAERWLKHKTITFEEI |
| | AGKGKNQLTFNQIALEEAGRYAAEDADVTLQLHLKMWPDLQKHKGPLNVFENIEMPLVPVLS |
| | RIERNGVKIDPKVLHNHSEELTLRLAELEKKAHEIAGEEFNLSSTKQLQTILFEKQGIKPLKKTPGG |
| | APSTSEEVLEELALDYPLPKVILEYRGLAKLKSTYTDKLPLMINPKTGRVHTSYHQAVTATGRLSST |
| | DPNLQNIPVRNEEGRRIRQAFIAPEDYVIVSADYSQIELRIMAHLSRDKGLLTAFAEGKDIHRATA |
| | AEVFGLPLETVTSEQRRSAKAINFGLIYGMSAFGLARQLNIPRKEAQKYMDLYFERYPGVLEYM |
| | ERTRAQAKEQGYVETLDGRRLYLPDIKSSNGARRAAAERAAINAPMQGTAADIIKRAMIAVDA |
| | WLQAEQPRVRMIMQVHDELVFEVHKDDVDAVAKQIHQLMENCTRLDVPLLVEVGSGENWD |
| | QAHSGGSKRTADGSEFESPKKKRKV (SEQ ID NO: 145) |
| ChiSCV- | MHLFTDVVSVDIMTTMQRTHTNAELIRWFKIFRDLDIHKWVIGLEEGKGGYGHWQVRCNVR |
| GM415- | VEVVNDWTAYLRAVFGWLGPISIWTEECSDKYTYETKEGKYWASWDTMGARQQRFGKMR |
| XTEN- | WNQEGAVQALQRTNDREIVVWYDEQGNMGKSWLCGHLFETGQAYYIPPYMTSIQSMIQTV |
| nCas9 | ASLVLQDRDSGYPPRPLIVIDIPRSWKWSTELYTAIEAIKDGLIMDPRYGARPVNIHGIKVIVLTNT |
| (H840A) | KPKLDKLSEDRWVLYDPMDYLMMLSGGSSGSETPGTSESATPESSGGSDKKYSIGLDIGTNSVG |
| SGGS- | WAVITDEYKVPSKKFKVLGNTDRHSIKKNLIGALLFDSGETAEATRLKRTARRRYTRRKNRICYLQ |
| (SEQ ID | EIFSNEMAKVDDSFFHRLEESFLVEEDKKHERHPIFGNIVDEVAYHEKYPTIYHLRKKLVDSTDKA |
| NO: | DLRLIYLALAHMIKFRGHFLIEGDLNPDNSDVDKLFIQLVQTYNQLFEENPINASGVDAKAILSAR |
| 573)_ | LSKSRRLENLIAQLPGEKKNGLFGNLIALSLGLTPNFKSNFDLAEDAKLQLSKDTYDDDLDNLLAQ |
| NLS_SGGS | IGDQYADLFLAAKNLSDAILLSDILRVNTEITKAPLSASMIKRYDEHHQDLTLLKALVRQQLPEKYK |
| (SEQ ID | EIFFDQSKNGYAGYIDGGASQEEFYKFIKPILEKMDGTEELLVKLNREDLLRKQRTFDNGSIPHQI |
| NO: | HLGELHAILRRQEDFYPFLKDNREKIEKILTFRIPYYVGPLARGNSRFAWMTRKSEETITPWNFEE |
| 573)- | VVDKGASAQSFIERMTNFDKNLPNEKVLPKHSLLYEYFTVYNELTKVKYVTEGMRKPAFLSGEQ |
| EcKLenow | KKAIVDLLFKTNRKVTVKQLKEDYFKKIECFDSVEISGVEDRFNASLGTYHDLLKIIKDKDFLDNEE |
| (D355A, | NEDILEDIVLTLTLFEDREMIEERLKTYAHLFDDKVMKQLKRRRYTGWGRLSRKLINGIRDKQSG |
| D357A)- | KTILDFLKSDGFANRNFMQLIHDDSLTFKEDIQKAQVSGQGDSLHEHIANLAGSPAIKKGILQTV |
| NLS | KVVDELVKVMGRHKPENIVIEMARENQTTQKGQKNSRERMKRIEEGIKELGSQILKEHPVENT |
| | QLQNEKLYLYYLQNGRDMYVDQELDINRLSDYDVDAIVPQSFLKDDSIDNKVLTRSDKNRGKS |
| | DNVPSEEVVKKMKNYWRQLLNAKLITQRKFDNLTKAERGGLSELDKAGFIKRQLVETRQITKHV |
| | AQILDSRMNTKYDENDKLIREVKVITLKSKLVSDFRKDFQFYKVREINNYHHAHDAYLNAVVGT |
| | ALIKKYPKLESEFVYGDYKVYDVRKMIAKSEQEIGKATAKYFFYSNIMNFFKTEITLANGEIRKRPLI |
| | ETNGETGEIVWDKGRDFATVRKVLSMPQVNIVKKTEVQTGGFSKESILPKRNSDKLIARKKDW |
| | DPKKYGGFDSPTVAYSVLVVAKVEKGKSKKLKSVKELLGITIMERSSFEKNPIDFLEAKGYKEVKK |
| | DLIIKLPKYSLFELENGRKRMLASAGELQKGNELALPSKYVNFLYLASHYEKLKGSPEDNEQKQLF |
| | VEQHKHYLDEIIEQISEFSKRVILADANLDKVLSAYNKHRDKPIREQAENIIHLFTLTNLGAPAAFK |
| | YFDTTIDRKRYTSTKEVLDATLIHQSITGLYETRIDLSQLGGDSGGSSGGSKRTADGSEFESPKKKR |
| | KVSGGSSGGSVISYDNYVTILDEETLKAWIAKLEKAPVFAFATATDSLDNISANLVGLSFAIEPGV |
| | AAYIPVAHDYLDAPDQISRERALELLKPLLEDEKALKVGQNLKYDRGILANYGIELRGIAFDTMLE |
| | SYILNSVAGRHDMDSLAERWLKHKTITFEEIAGKGKNQLTFNQIALEEAGRYAAEDADVTLQLH |
| | LKMWPDLQKHKGPLNVFENIEMPLVPVLSRIERNGVKIDPKVLHNHSEELTLRLAELEKKAHEIA |
| | GEEFNLSSTKQLQTILFEKQGIKPLKKTPGGAPSTSEEVLEELALDYPLPKVILEYRGLAKLKSTYTD |
| | KLPLMINPKTGRVHTSYHQAVTATGRLSSTDPNLQNIPVRNEEGRRIRQAFIAPEDYVIVSADYS |
| | QIELRIMAHLSRDKGLLTAFAEGKDIHRATAAEVFGLPLETVTSEQRRSAKAINFGLIYGMSAFGL |
| | ARQLNIPRKEAQKYMDLYFERYPGVLEYMERTRAQAKEQGYVETLDGRRLYLPDIKSSNGARR |
| | AAAERAAINAPMQGTAADIIKRAMIAVDAWLQAEQPRVRMIMQVHDELVFEVHKDDVDAV |
| | AKQIHQLMENCTRLDVPLLVEVGSGENWDQAHSGGSKRTADGSEFESPKKKRKV (SEQ ID |
| | NO: 146) |
| ChiSCV- | MHLFTEGISVDIMTTMQRTHTNAELIRWFKIFRDLDIHKWVIGLEEGKGEYGHWQVRCNVRV |
| GM510- | EVVNDWTAYLRAVFGWLGPISIWTEECSDKYTYETKEGKYWASWDTMGARQQRFGKMRW |
| XTEN- | NQEGAVQALQRTNDREIVVWYDEGGNMGKSWLCGHLFETGQAYYIPPYMTSIQSMIQTVAS |
| nCas9 | LVLQDRETGYPPRPLIVIDIPRSWKWSTELYTAIEAIKGGLIMDPRYGARPVNIHGTKVIVLTNTK |
| (H840A)- | PKLDKLSEDRWVLYDPMDYLMMLSGGSSGSETPGTSESATPESSGGSDKKYSIGLDIGTNSVG |
| SGGS | WAVITDEYKVPSKKFKVLGNTDRHSIKKNLIGALLFDSGETAEATRLKRTARRRYTRRKNRICYLQ |
| (SEQ ID | EIFSNEMAKVDDSFFHRLEESFLVEEDKKHERHPIFGNIVDEVAYHEKYPTIYHLRKKLVDSTDKA |
| NO: | DLRLIYLALAHMIKFRGHFLIEGDLNPDNSDVDKLFIQLVQTYNQLFEENPINASGVDAKAILSAR |
| 573)_ | LSKSRRLENLIAQLPGEKKNGLFGNLIALSLGLTPNFKSNFDLAEDAKLQLSKDTYDDDLDNLLAQ |
| NLS_SGGS | IGDQYADLFLAAKNLSDAILLSDILRVNTEITKAPLSASMIKRYDEHHQDLTLLKALVRQQLPEKYK |
| (SEQ ID | EIFFDQSKNGYAGYIDGGASQEEFYKFIKPILEKMDGTEELLVKLNREDLLRKQRTFDNGSIPHQI |
| NO: | HLGELHAILRRQEDFYPFLKDNREKIEKILTFRIPYYVGPLARGNSRFAWMTRKSEETITPWNFEE |
| 573)- | VVDKGASAQSFIERMTNFDKNLPNEKVLPKHSLLYEYFTVYNELTKVKYVTEGMRKPAFLSGEQ |
| EckLenow | KKAIVDLLFKTNRKVTVKQLKEDYFKKIECFDSVEISGVEDRFNASLGTYHDLLKIIKDKDFLDNEE |
| (D355A, | NEDILEDIVLTLTLFEDREMIEERLKTYAHLFDDKVMKQLKRRRYTGWGRLSRKLINGIRDKQSG |
| D357A)- | KTILDFLKSDGFANRNFMQLIHDDSLTFKEDIQKAQVSGQGDSLHEHIANLAGSPAIKKGILQTV |
| NLS | KVVDELVKVMGRHKPENIVIEMARENQTTQKGQKNSRERMKRIEEGIKELGSQILKEHPVENT |
| | QLQNEKLYLYYLQNGRDMYVDQELDINRLSDYDVDAIVPQSFLKDDSIDNKVLTRSDKNRGKS |
| | DNVPSEEVVKKMKNYWRQLLNAKLITQRKFDNLTKAERGGLSELDKAGFIKRQLVETRQITKHV |
| | AQILDSRMNTKYDENDKLIREVKVITLKSKLVSDFRKDFQFYKVREINNYHHAHDAYLNAVVGT |
| | ALIKKYPKLESEFVYGDYKVYDVRKMIAKSEQEIGKATAKYFFYSNIMNFFKTEITLANGEIRKRPLI |
| | ETNGETGEIVWDKGRDFATVRKVLSMPQVNIVKKTEVQTGGFSKESILPKRNSDKLIARKKDW |
| | DPKKYGGFDSPTVAYSVLVVAKVEKGKSKKLKSVKELLGITIMERSSFEKNPIDFLEAKGYKEVKK |
| | DLIIKLPKYSLFELENGRKRMLASAGELQKGNELALPSKYVNFLYLASHYEKLKGSPEDNEQKQLF |
| | VEQHKHYLDEIIEQISEFSKRVILADANLDKVLSAYNKHRDKPIREQAENIIHLFTLTNLGAPAAFK |
| | YFDTTIDRKRYTSTKEVLDATLIHQSITGLYETRIDLSQLGGDSGGSSGGSKRTADGSEFESPKKKR |
| | KVSGGSSGGSVISYDNYVTILDEETLKAWIAKLEKAPVFAFATATDSLDNISANLVGLSFAIEPGV |
| | AAYIPVAHDYLDAPDQISRERALELLKPLLEDEKALKVGQNLKYDRGILANYGIELRGIAFDTMLE |
| | SYILNSVAGRHDMDSLAERWLKHKTITFEEIAGKGKNQLTFNQIALEEAGRYAAEDADVTLQLH |
| | LKMWPDLQKHKGPLNVFENIEMPLVPVLSRIERNGVKIDPKVLHNHSEELTLRLAELEKKAHEIA |
| | GEEFNLSSTKQLQTILFEKQGIKPLKKTPGGAPSTSEEVLEELALDYPLPKVILEYRGLAKLKSTYTD |
| | KLPLMINPKTGRVHTSYHQAVTATGRLSSTDPNLQNIPVRNEEGRRIRQAFIAPEDYVIVSADYS |
| | QIELRIMAHLSRDKGLLTAFAEGKDIHRATAAEVFGLPLETVTSEQRRSAKAINFGLIYGMSAFGL |
| | ARQLNIPRKEAQKYMDLYFERYPGVLEYMERTRAQAKEQGYVETLDGRRLYLPDIKSSNGARR |
| | AAAERAAINAPMQGTAADIIKRAMIAVDAWLQAEQPRVRMIMQVHDELVFEVHKDDVDAV |
| | AKQIHQLMENCTRLDVPLLVEVGSGENWDQAHSGGSKRTADGSEFESPKKKRKV (SEQ ID |
| | NO: 147) |
| ChiSCV- | MGARQQRFGKMRWNQEGALQALQRTNDREIVVWYDQDGNMGKSWLCGHLYETGQAYYI |
| GT306- | PPYMTSIQSMIQTVASLVLQDRESGYPPRPLIVIDIPRSRKWSTELYTAIEAIKDGLIMDPRYGAR |
| XTEN- | PVNIHGTKVIVLTNTKPKLDKLSEDRWVLYDPLLYMMGHSGGSSGSETPGTSESATPESSGGSD |
| nCas9 | KKYSIGLDIGTNSVGWAVITDEYKVPSKKFKVLGNTDRHSIKKNLIGALLFDSGETAEATRLKRTA |
| (H840A)- | RRRYTRRKNRICYLQEIFSNEMAKVDDSFFHRLEESFLVEEDKKHERHPIFGNIVDEVAYHEKYPT |
| SGGS | IYHLRKKLVDSTDKADLRLIYLALAHMIKFRGHFLIEGDLNPDNSDVDKLFIQLVQTYNQLFEENP |
| (SEQ ID | INASGVDAKAILSARLSKSRRLENLIAQLPGEKKNGLFGNLIALSLGLTPNFKSNFDLAEDAKLQLS |
| NO: | KDTYDDDLDNLLAQIGDQYADLFLAAKNLSDAILLSDILRVNTEITKAPLSASMIKRYDEHHQDLT |
| 573)_ | LLKALVRQQLPEKYKEIFFDQSKNGYAGYIDGGASQEEFYKFIKPILEKMDGTEELLVKLNREDLL |
| NLS_SGGS | RKQRTFDNGSIPHQIHLGELHAILRRQEDFYPFLKDNREKIEKILTFRIPYYVGPLARGNSRFAWM |
| (SEQ ID | TRKSEETITPWNFEEVVDKGASAQSFIERMTNFDKNLPNEKVLPKHSLLYEYFTVYNELTKVKYV |
| NO: | TEGMRKPAFLSGEQKKAIVDLLFKTNRKVTVKQLKEDYFKKIECFDSVEISGVEDRFNASLGTYH |
| 573)- | DLLKIIKDKDFLDNEENEDILEDIVLTLTLFEDREMIEERLKTYAHLFDDKVMKQLKRRRYTGWGR |
| EckLenow | LSRKLINGIRDKQSGKTILDFLKSDGFANRNFMQLIHDDSLTFKEDIQKAQVSGQGDSLHEHIAN |
| (D355A, | LAGSPAIKKGILQTVKVVDELVKVMGRHKPENIVIEMARENQTTQKGQKNSRERMKRIEEGIKE |
| D357A)- | LGSQILKEHPVENTQLQNEKLYLYYLQNGRDMYVDQELDINRLSDYDVDAIVPQSFLKDDSIDN |
| NLS | KVLTRSDKNRGKSDNVPSEEVVKKMKNYWRQLLNAKLITQRKFDNLTKAERGGLSELDKAGFIK |
| | RQLVETRQITKHVAQILDSRMNTKYDENDKLIREVKVITLKSKLVSDFRKDFQFYKVREINNYHH |
| | AHDAYLNAVVGTALIKKYPKLESEFVYGDYKVYDVRKMIAKSEQEIGKATAKYFFYSNIMNFFKT |
| | EITLANGEIRKRPLIETNGETGEIVWDKGRDFATVRKVLSMPQVNIVKKTEVQTGGFSKESILPKR |
| | NSDKLIARKKDWDPKKYGGFDSPTVAYSVLVVAKVEKGKSKKLKSVKELLGITIMERSSFEKNPID |
| | FLEAKGYKEVKKDLIIKLPKYSLFELENGRKRMLASAGELQKGNELALPSKYVNFLYLASHYEKLKG |
| | SPEDNEQKQLFVEQHKHYLDEIIEQISEFSKRVILADANLDKVLSAYNKHRDKPIREQAENIIHLFT |
| | LTNLGAPAAFKYFDTTIDRKRYTSTKEVLDATLIHQSITGLYETRIDLSQLGGDSGGSSGGSKRTA |
| | DGSEFESPKKKRKVSGGSSGGSVISYDNYVTILDEETLKAWIAKLEKAPVFAFATATDSLDNISAN |
| | LVGLSFAIEPGVAAYIPVAHDYLDAPDQISRERALELLKPLLEDEKALKVGQNLKYDRGILANYGIE |
| | LRGIAFDTMLESYILNSVAGRHDMDSLAERWLKHKTITFEEIAGKGKNQLTFNQIALEEAGRYA |
| | AEDADVTLQLHLKMWPDLQKHKGPLNVFENIEMPLVPVLSRIERNGVKIDPKVLHNHSEELTLR |
| | LAELEKKAHEIAGEEFNLSSTKQLQTILFEKQGIKPLKKTPGGAPSTSEEVLEELALDYPLPKVILEY |
| | RGLAKLKSTYTDKLPLMINPKTGRVHTSYHQAVTATGRLSSTDPNLQNIPVRNEEGRRIRQAFIA |
| | PEDYVIVSADYSQIELRIMAHLSRDKGLLTAFAEGKDIHRATAAEVFGLPLETVTSEQRRSAKAIN |
| | FGLIYGMSAFGLARQLNIPRKEAQKYMDLYFERYPGVLEYMERTRAQAKEQGYVETLDGRRLYL |
| | PDIKSSNGARRAAAERAAINAPMQGTAADIIKRAMIAVDAWLQAEQPRVRMIMQVHDELVF |
| | EVHKDDVDAVAKQIHQLMENCTRLDVPLLVEVGSGENWDQAHSGGSKRTADGSEFESPKKK |
| | RKV (SEQ ID NO: 148) |
| Ecklenow- | MVISYDNYVTILDEETLKAWIAKLEKAPVFAFATATDSLDNISANLVGLSFAIEPGVAAYIPVAHD |
| NLS- | YLDAPDQISRERALELLKPLLEDEKALKVGQNLKYDRGILANYGIELRGIAFDTMLESYILNSVAGR |
| CC(N5) | HDMDSLAERWLKHKTITFEEIAGKGKNQLTFNQIALEEAGRYAAEDADVTLQLHLKMWPDLQ |
| | KHKGPLNVFENIEMPLVPVLSRIERNGVKIDPKVLHNHSEELTLRLAELEKKAHEIAGEEFNLSST |
| | KQLQTILFEKQGIKPLKKTPGGAPSTSEEVLEELALDYPLPKVILEYRGLAKLKSTYTDKLPLMINPK |
| | TGRVHTSYHQAVTATGRLSSTDPNLQNIPVRNEEGRRIRQAFIAPEDYVIVSADYSQIELRIMAH |
| | LSRDKGLLTAFAEGKDIHRATAAEVFGLPLETVTSEQRRSAKAINFGLIYGMSAFGLARQLNIPRK |
| | EAQKYMDLYFERYPGVLEYMERTRAQAKEQGYVETLDGRRLYLPDIKSSNGARRAAAERAAIN |
| | APMQGTAADIIKRAMIAVDAWLQAEQPRVRMIMQVHDELVFEVHKDDVDAVAKQIHQLME |
| | NCTRLDVPLLVEVGSGENWDQAHSGGSSGGSKRTADGSEFESPKKKRKVSGGSSGGSGEIAAL |
| | EAKIAALKAKNAALKAEIAALEAGY (SEQ ID NO: 149) |
| PCV2- | MSPSKKNGRSGPQPHKRWVFTLNNPSEDERKKIRDLPISLFDYFIVGEEGNEEGRTPHLQGFAN |
| nCas9 | FVKKQTFNKVKWYLGARCHIEKAKGTDQQNKEYCSKEGNLLMECGAPRSQGQRSGGSSGSET |
| (H840A)- | PGTSESATPESSGGSDKKYSIGLDIGTNSVGWAVITDEYKVPSKKFKVLGNTDRHSIKKNLIGALL |
| NLS | FDSGETAEATRLKRTARRRYTRRKNRICYLQEIFSNEMAKVDDSFFHRLEESFLVEEDKKHERHPI |
| | FGNIVDEVAYHEKYPTIYHLRKKLVDSTDKADLRLIYLALAHMIKFRGHFLIEGDLNPDNSDVDKL |
| | FIQLVQTYNQLFEENPINASGVDAKAILSARLSKSRRLENLIAQLPGEKKNGLFGNLIALSLGLTPN |
| | FKSNFDLAEDAKLQLSKDTYDDDLDNLLAQIGDQYADLFLAAKNLSDAILLSDILRVNTEITKAPL |
| | SASMIKRYDEHHQDLTLLKALVRQQLPEKYKEIFFDQSKNGYAGYIDGGASQEEFYKFIKPILEK |
| | MDGTEELLVKLNREDLLRKQRTFDNGSIPHQIHLGELHAILRRQEDFYPFLKDNREKIEKILTFRIP |
| | YYVGPLARGNSRFAWMTRKSEETITPWNFEEVVDKGASAQSFIERMTNFDKNLPNEKVLPKHS |
| | LLYEYFTVYNELTKVKYVTEGMRKPAFLSGEQKKAIVDLLFKTNRKVTVKQLKEDYFKKIECFDSV |
| | EISGVEDRFNASLGTYHDLLKIIKDKDFLDNEENEDILEDIVLTLTLFEDREMIEERLKTYAHLFDDK |
| | VMKQLKRRRYTGWGRLSRKLINGIRDKQSGKTILDFLKSDGFANRNFMQLIHDDSLTFKEDIQK |
| | AQVSGQGDSLHEHIANLAGSPAIKKGILQTVKVVDELVKVMGRHKPENIVIEMARENQTTQKG |
| | QKNSRERMKRIEEGIKELGSQILKEHPVENTQLQNEKLYLYYLQNGRDMYVDQELDINRLSDYD |
| | VDAIVPQSFLKDDSIDNKVLTRSDKNRGKSDNVPSEEVVKKMKNYWRQLLNAKLITQRKFDNL |
| | TKAERGGLSELDKAGFIKRQLVETRQITKHVAQILDSRMNTKYDENDKLIREVKVITLKSKLVSDF |
| | RKDFQFYKVREINNYHHAHDAYLNAVVGTALIKKYPKLESEFVYGDYKVYDVRKMIAKSEQEIG |
| | KATAKYFFYSNIMNFFKTEITLANGEIRKRPLIETNGETGEIVWDKGRDFATVRKVLSMPQVNIV |
| | KKTEVQTGGFSKESILPKRNSDKLIARKKDWDPKKYGGFDSPTVAYSVLVVAKVEKGKSKKLKSV |
| | KELLGITIMERSSFEKNPIDFLEAKGYKEVKKDLIIKLPKYSLFELENGRKRMLASAGELQKGNELA |
| | LPSKYVNFLYLASHYEKLKGSPEDNEQKQLFVEQHKHYLDEIIEQISEFSKRVILADANLDKVLSAY |
| | NKHRDKPIREQAENIIHLFTLTNLGAPAAFKYFDTTIDRKRYTSTKEVLDATLIHQSITGLYETRIDL |
| | SQLGGDSGGSSGGSKRTADGSEFESPKKKRKV (SEQ ID NO: 150) |
| PCV2- | MSPSKKNGRSGPQPHKRWVFTLNNPSEDERKKIRDLPISLFDYFIVGEEGNEEGRTPHLQGFAN |
| nCas9 | FVKKQTFNKVKWYLGARCHIEKAKGTDQQNKEYCSKEGNLLMECGAPRSQGQRSGGSSGSET |
| (H840A)- | PGTSESATPESSGGSDKKYSIGLDIGTNSVGWAVITDEYKVPSKKFKVLGNTDRHSIKKNLIGALL |
| NLS- | FDSGETAEATRLKRTARRRYTRRKNRICYLQEIFSNEMAKVDDSFFHRLEESFLVEEDKKHERHPI |
| CC(N6) | FGNIVDEVAYHEKYPTIYHLRKKLVDSTDKADLRLIYLALAHMIKFRGHFLIEGDLNPDNSDVDKL |
| | FIQLVQTYNQLFEENPINASGVDAKAILSARLSKSRRLENLIAQLPGEKKNGLFGNLIALSLGLTPN |
| | FKSNFDLAEDAKLQLSKDTYDDDLDNLLAQIGDQYADLFLAAKNLSDAILLSDILRVNTEITKAPL |
| | SASMIKRYDEHHQDLTLLKALVRQQLPEKYKEIFFDQSKNGYAGYIDGGASQEEFYKFIKPILEK |
| | MDGTEELLVKLNREDLLRKQRTFDNGSIPHQIHLGELHAILRRQEDFYPFLKDNREKIEKILTFRIP |
| | YYVGPLARGNSRFAWMTRKSEETITPWNFEEVVDKGASAQSFIERMTNFDKNLPNEKVLPKHS |
| | LLYEYFTVYNELTKVKYVTEGMRKPAFLSGEQKKAIVDLLFKTNRKVTVKQLKEDYFKKIECFDSV |
| | EISGVEDRFNASLGTYHDLLKIIKDKDFLDNEENEDILEDIVLTLTLFEDREMIEERLKTYAHLFDDK |
| | VMKQLKRRRYTGWGRLSRKLINGIRDKQSGKTILDFLKSDGFANRNFMQLIHDDSLTFKEDIQK |
| | AQVSGQGDSLHEHIANLAGSPAIKKGILQTVKVVDELVKVMGRHKPENIVIEMARENQTTQKG |
| | QKNSRERMKRIEEGIKELGSQILKEHPVENTQLQNEKLYLYYLQNGRDMYVDQELDINRLSDYD |
| | VDAIVPQSFLKDDSIDNKVLTRSDKNRGKSDNVPSEEVVKKMKNYWRQLLNAKLITQRKFDNL |
| | TKAERGGLSELDKAGFIKRQLVETRQITKHVAQILDSRMNTKYDENDKLIREVKVITLKSKLVSDF |
| | RKDFQFYKVREINNYHHAHDAYLNAVVGTALIKKYPKLESEFVYGDYKVYDVRKMIAKSEQEIG |
| | KATAKYFFYSNIMNFFKTEITLANGEIRKRPLIETNGETGEIVWDKGRDFATVRKVLSMPQVNIV |
| | KKTEVQTGGFSKESILPKRNSDKLIARKKDWDPKKYGGFDSPTVAYSVLVVAKVEKGKSKKLKSV |
| | KELLGITIMERSSFEKNPIDFLEAKGYKEVKKDLIIKLPKYSLFELENGRKRMLASAGELQKGNELA |
| | LPSKYVNFLYLASHYEKLKGSPEDNEQKQLFVEQHKHYLDEIIEQISEFSKRVILADANLDKVLSAY |
| | NKHRDKPIREQAENIIHLFTLTNLGAPAAFKYFDTTIDRKRYTSTKEVLDATLIHQSITGLYETRIDL |
| | SQLGGDSGGSSGGSKRTADGSEFESPKKKRKVSGGSSGGSKIAALKAEIAALEAENAALEAKIAA |
| | LKAG (SEQ ID NO: 151) |
| Phi29 | MKHMPRKMYSCDFETTTKVEDCRVWAYGYMNIEDHSEYKIGNSLDEFMAWVLKVQADLYFH |
| (D169A)- | NLKFDGAFIINWLERNGFKWSADGLPNTYNTIISRMGQWYMIDICLGYKGKRKIHTVIYDSLKK |
| NLS | LPFPVKKIAKDFKLTVLKGDIDYHKERPVGYKITPEEYAYIKNAIQIIAEALLIQFKQGLDRMTAGS |
| | DSLKGFKDIITTKKFKKVFPTLSLGLDKEVRYAYRGGFTWLNDRFKEKEIGEGMVFDVNSLYPAQ |
| | MYSRLLPYGEPIVFEGKYVWDEDYPLHIQHIRCEFELKEGYIPTIQIKRSRFYKGNEYLKSSGGEIA |
| | DLWLSNVDLELMKEHYDLYNVEYISGLKFKATTGLFKDFIDKWTYIKTTSEGAIKQLAKLMLNSL |
| | YGKFASNPDVTGKVPYLKENGALGFRLGEEETKDPVYTPMGVFITAWARYTTITAAQACYDRIIY |
| | CDTDSIHLTGTEIPDVIKDIVDPKKLGYWAHESTFKRAKYLRQKTYIQDIYMKEVDGKLVEGSPD |
| | DYTDIKFSVKCAGMTDKIKKEVTFENFKVGFSRKMKPKPVQVPGGVVLVDDTFTIKSGGSSGGS |
| | KRTADGSEFESPKKKRKV (SEQ ID NO: 152) |
| Phi29 | MKHMPRKRYSCDFETTTKVEDCRVWAYGYMNIEDHSEYKIGNSLDEFMAWALKVQADLYFH |
| (D169A, | NLKFDGAFIINWLERNGFKWSADGLPNTYNTIISRTGQWYMIDICLGYKGKRKIHTVIYDSLKKL |
| M8R, V51A, | PFPVKKIAKDFKLTVLKGDIDYHKERPVGYKITPEEYAYIKNAIQIIAEALLIQFKQGLDRMTAGSD |
| M97T, | SLKDFKDIITTKKFKKVFPTLSLGLDKKVRYAYRGGFTWLNDRFKEKEIGEGMVFDVNSLYPAQM |
| G197D, | YSRLLPYGEPIVFEGKYVWDEDYPLHIQHIRCEFELKEGYIPTIQIKRSRFYKGNEYLKSSGGEIADL |
| E221K, | WLSNVDLELMKEHYDLYNVEYISGLKFKATTGLFKDFIDKWTYIKTTSEGAIKQLAKLMLNSLYG |
| Q497P, | KFASNPDVTGKVPYLKENGALGFRLGEEETKDPVYTPMGVFITAWARYTTITAAQACYDRIIYC |
| K512E, | DTDSIHLTGTEIPDVIKDIVDPKKLGYWAHESTFKRAKYLRPKTYIQDIYMKEVDGELVEGSPDDY |
| F526L)- | TDIKLSVKCAGMTDKIKKEVTFENFKVGFSRKMKPKPVQVPGGVVLVDDTFTIKSGGSSGGSKR |
| NLS- | TADGSEFESPKKKRKVSGGSSGGSGEIAALEAKIAALKAKNAALKAEIAALEAGY (SEQ ID |
| linker- | NO: 153) |
| CC(N5) | |
| Phi29 | MKHMPRKMYSCDFETTTKVEDCRVWAYGYMNIEDHSEYKIGNSLDEFMAWVLKVQADLYFH |
| (D169A)- | NLKFDGAFIINWLERNGFKWSADGLPNTYNTIISRMGQWYMIDICLGYKGKRKIHTVIYDSLKK |
| NLS- | LPFPVKKIAKDFKLTVLKGDIDYHKERPVGYKITPEEYAYIKNAIQIIAEALLIQFKQGLDRMTAGS |
| linker- | DSLKGFKDIITTKKFKKVFPTLSLGLDKEVRYAYRGGFTWLNDRFKEKEIGEGMVFDVNSLYPAQ |
| CC(N5) | MYSRLLPYGEPIVFEGKYVWDEDYPLHIQHIRCEFELKEGYIPTIQIKRSRFYKGNEYLKSSGGEIA |
| | DLWLSNVDLELMKEHYDLYNVEYISGLKFKATTGLFKDFIDKWTYIKTTSEGAIKQLAKLMLNSL |
| | YGKFASNPDVTGKVPYLKENGALGFRLGEEETKDPVYTPMGVFITAWARYTTITAAQACYDRIIY |
| | CDTDSIHLTGTEIPDVIKDIVDPKKLGYWAHESTFKRAKYLRQKTYIQDIYMKEVDGKLVEGSPD |
| | DYTDIKFSVKCAGMTDKIKKEVTFENFKVGFSRKMKPKPVQVPGGVVLVDDTFTIKSGGSSGGS |
| | KRTADGSEFESPKKKRKVSGGSSGGSGEIAALEAKIAALKAKNAALKAEIAALEAGY (SEQ ID |
| | NO: 154) |
| Phi29- | MKHMPRKMYSCDFETTTKVEDCRVWAYGYMNIEDHSEYKIGNSLDEFMAWVLKVQADLYFH |
| NLS | NLKFDGAFIINWLERNGFKWSADGLPNTYNTIISRMGQWYMIDICLGYKGKRKIHTVIYDSLKK |
| | LPFPVKKIAKDFKLTVLKGDIDYHKERPVGYKITPEEYAYIKNDIQIIAEALLIQFKQGLDRMTAGS |
| | DSLKGFKDIITTKKFKKVFPTLSLGLDKEVRYAYRGGFTWLNDRFKEKEIGEGMVFDVNSLYPAQ |
| | MYSRLLPYGEPIVFEGKYVWDEDYPLHIQHIRCEFELKEGYIPTIQIKRSRFYKGNEYLKSSGGEIA |
| | DLWLSNVDLELMKEHYDLYNVEYISGLKFKATTGLFKDFIDKWTYIKTTSEGAIKQLAKLMLNSL |
| | YGKFASNPDVTGKVPYLKENGALGFRLGEEETKDPVYTPMGVFITAWARYTTITAAQACYDRIIY |
| | CDTDSIHLTGTEIPDVIKDIVDPKKLGYWAHESTFKRAKYLRQKTYIQDIYMKEVDGKLVEGSPD |
| | DYTDIKFSVKCAGMTDKIKKEVTFENFKVGFSRKMKPKPVQVPGGVVLVDDTFTIKSGGSSGGS |
| | KRTADGSEFESPKKKRKV (SEQ ID NO: 155) |
| Phi29- | MKHMPRKMYSCDFETTTKVEDCRVWAYGYMNIEDHSEYKIGNSLDEFMAWVLKVQADLYFH |
| NLS- | NLKFDGAFIINWLERNGFKWSADGLPNTYNTIISRMGQWYMIDICLGYKGKRKIHTVIYDSLKK |
| CC(N5) | LPFPVKKIAKDFKLTVLKGDIDYHKERPVGYKITPEEYAYIKNDIQIIAEALLIQFKQGLDRMTAGS |
| | DSLKGFKDIITTKKFKKVFPTLSLGLDKEVRYAYRGGFTWLNDRFKEKEIGEGMVFDVNSLYPAQ |
| | MYSRLLPYGEPIVFEGKYVWDEDYPLHIQHIRCEFELKEGYIPTIQIKRSRFYKGNEYLKSSGGEIA |
| | DLWLSNVDLELMKEHYDLYNVEYISGLKFKATTGLFKDFIDKWTYIKTTSEGAIKQLAKLMLNSL |
| | YGKFASNPDVTGKVPYLKENGALGFRLGEEETKDPVYTPMGVFITAWARYTTITAAQACYDRIIY |
| | CDTDSIHLTGTEIPDVIKDIVDPKKLGYWAHESTFKRAKYLRQKTYIQDIYMKEVDGKLVEGSPD |
| | DYTDIKFSVKCAGMTDKIKKEVTFENFKVGFSRKMKPKPVQVPGGVVLVDDTFTIKSGGSSGGS |
| | KRTADGSEFESPKKKRKVSGGSSGGSGEIAALEAKIAALKAKNAALKAEIAALEAGY (SEQ ID |
| | NO: 156) |
| Phi29 | MKHMPRKRYSCDFETTTKVEDCRVWAYGYMNIEDHSEYKIGNSLDEFMAWALKVQADLYFH |
| (D169A, | NLKFDGAFIINWLERNGFKWSADGLPNTYNTIISRTGQWYMIDICLGYKGKRKIHTVIYDSLKKL |
| M8R, V51A, | PFPVKKIAKDFKLTVLKGDIDYHKERPVGYKITPEEYAYIKNAIQIIAEALLIQFKQGLDRMTAGSD |
| M97T, | SLKDFKDIITTKKFKKVFPTLSLGLDKKVRYAYRGGFTWLNDRFKEKEIGEGMVFDVNSLYPAQM |
| G197D, | YSRLLPYGEPIVFEGKYVWDEDYPLHIQHIRCEFELKEGYIPTIQIKRSRFYKGNEYLKSSGGEIADL |
| E221K, | WLSNVDLELMKEHYDLYNVEYISGLKFKATTGLFKDFIDKWTYIKTTSEGAIKQLAKLMLNSLYG |
| Q497P, | KFASNPDVTGKVPYLKENGALGFRLGEEETKDPVYTPMGVFITAWARYTTITAAQACYDRIIYC |
| K512E, | DTDSIHLTGTEIPDVIKDIVDPKKLGYWAHESTFKRAKYLRPKTYIQDIYMKEVDGELVEGSPDDY |
| F526L)- | TDIKLSVKCAGMTDKIKKEVTFENFKVGFSRKMKPKPVQVPGGVVLVDDTFTIKSGGSSGGSKR |
| NLS | TADGSEFESPKKKRKV (SEQ ID NO: 157) |
| B103 | MPRKMFSCDFETTTKLDDCRVWAYGYMEIGNLDNYKIGNSLDEFMQWVMEIQADLYFHNLK |
| (H73R, | FDGAFIVNWLERHGFKWSNEGLPNTYNTIISKMGQWYMIDICFGYKGKRKLHTVIYDSLKKLPF |
| A147K, | PVKKIAKDFQLPLLKGDIDYHKERPVGHEITPEEYEYIKNDIEIIARALDIQFKQGLDRMTAGSDSL |
| R221Y, | KGFKDILSTKKFNKVFPKLSLPMDKEIRYAYRGGFTWLNDKYKEKEIGEGMVFDVNSLYPSQMY |
| A318G, | SRPLPYGAPIVFQGKYEKDEQYPLYIQRIRFEFELKEGYIPTIQIKKNPFFKGNEYLKNSGGEPVELY |
| M339L, | LTNVDLELIQEHYELYNVEYIDGFKFREKTGLFKDFIDKWTYVKTHEEGAKKQLAKLMLNSLYGK |
| E359D, | FASNPDVTGKVPYLKEDGSLGFRVGDEEYKDPVYTPMGVFITAWARFTTITAAQACYDRIIYCD |
| K372E, | TDSIHLTGTEVPEIIKDIVDPKKLGYWAHESTFKRAKYLRQKTYIQDIYMKEVDGKLVECSPDEAT |
| F383L, | TTKFSVKCAGMTDTIKKKVTFDNFKVGFSSKGKPKPVQVNGGVVLVDSVFTIKSGGSSGGSKRT |
| D384N, | ADGSEFESPKKKRKV (SEQ ID NO: 158) |
| A503M, | |
| I511V, | |
| R544K, | |
| T550K)- | |
| NLS | |
| PCV2- | MSPSKKNGRSGPQPHKRWVFTLNNPSEDERKKIRDLPISLFDYFIVGEEGNEEGRTPHLQGFAN |
| nCas8 | FVKKQTFNKVKWYLGARCHIEKAKGTDQQNKEYCSKEGNLLMECGAPRSQGQRSGGSSGSET |
| (H840A)- | PGTSESATPESSGGSDKKYSIGLDIGTNSVGWAVITDEYKVPSKKFKVLGNTDRHSIKKNLIGALL |
| Phi29- | FDSGETAEATRLKRTARRRYTRRKNRICYLQEIFSNEMAKVDDSFFHRLEESFLVEEDKKHERHPI |
| NLS | FGNIVDEVAYHEKYPTIYHLRKKLVDSTDKADLRLIYLALAHMIKFRGHFLIEGDLNPDNSDVDKL |
| | FIQLVQTYNQLFEENPINASGVDAKAILSARLSKSRKLENLIAQLPGEKKNGLFGNLIALSLGLTPN |
| | FKSNFDLAEDAKLQLSKDTYDDDLDNLLAQIGDQYADLFLAAKNLSDAILLSDILRVNTEITKAPL |
| | SASMIKRYDEHHQDLTLLKALVRQQLPEKYKEIFFDQSKNGYAGYIDGGASQEEFYKFIKPILEK |
| | MDGTEELLVKLKREDLLRKQRTFDNGSIPHQIHLGELHAILRRQEDFYPFLKDNREKIEKILTFRIP |
| | YYVGPLARGNSRFAWMTRKSEETITPWNFEEVVDKGASAQSFIERMTNFDKNLPNEKVLPKHS |
| | LLYEYFTVYNELTKVKYVTEGMRKPAFLSGEQKKAIVDLLFKTNRKVTVKQLKEDYFKKIECFDSV |
| | EISGVEDRFNASLGTYHDLLKIIKDKDFLDNEENEDILEDIVLTLTLFEDREMIEERLKTYAHLFDDK |
| | VMKQLKRRRYTGWGRLSRKLINGIRDKQSGKTILDFLKSDGFANRNFMQLIHDDSLTFKEDIQK |
| | AQVSGQGDSLHEHIANLAGSPAIKKGILQTVKVVDELVKVMGRHKPENIVIEMARENQTTQKG |
| | QKNSRERMKRIEEGIKELGSQILKEHPVENTQLQNEKLYLYYLQNGRDMYVDQELDINRLSDYD |
| | VDAIVPQSFLKDDSIDNKVLTRSDKNRGKSDNVPSEEVVKKMKNYWRQLLNAKLITQRKFDNL |
| | TKAERGGLSELDKAGFIKRQLVETRQITKHVAQILDSRMNTKYDENDKLIREVKVITLKSKLVSDF |
| | RKDFQFYKVREINNYHHAHDAYLNAVVGTALIKKYPKLESEFVYGDYKVYDVRKMIAKSEQEIG |
| | KATAKYFFYSNIMNFFKTEITLANGEIRKRPLIETNGETGEIVWDKGRDFATVRKVLSMPQVNIV |
| | KKTEVQTGGFSKESILPKRNSDKLIARKKDWDPKKYGGFDSPTVAYSVLVVAKVEKGKSKKLKSV |
| | KELLGITIMERSSFEKNPIDFLEAKGYKEVKKDLIIKLPKYSLFELENGRKRMLASAGELQKGNELA |
| | LPSKYVNFLYLASHYEKLKGSPEDNEQKQLFVEQHKHYLDEIIEQISEFSKRVILADANLDKVLSAY |
| | NKHRDKPIREQAENIIHLFTLTNLGAPAAFKYFDTTIDRKRYTSTKEVLDATLIHQSITGLYETRIDL |
| | SQLGGDSGGSSGGSKRTADGSEFESPKKKRKVSGGSSGGSKHMPRKMYSCDFETTTKVEDCRV |
| | WAYGYMNIEDHSEYKIGNSLDEFMAWVLKVQADLYFHNLKFDGAFIINWLERNGFKWSADG |
| | LPNTYNTIISRMGQWYMIDICLGYKGKRKIHTVIYDSLKKLPFPVKKIAKDFKLTVLKGDIDYHKER |
| | PVGYKITPEEYAYIKNDIQIIAEALLIQFKQGLDRMTAGSDSLKGFKDIITTKKFKKVFPTLSLGLDK |
| | EVRYAYRGGFTWLNDRFKEKEIGEGMVFDVNSLYPAQMYSRLLPYGEPIVFEGKYVWDEDYPL |
| | HIQHIRCEFELKEGYIPTIQIKRSRFYKGNEYLKSSGGEIADLWLSNVDLELMKEHYDLYNVEYISG |
| | LKFKATTGLFKDFIDKWTYIKTTSEGAIKQLAKLMLNSLYGKFASNPDVTGKVPYLKENGALGFRL |
| | GEEETKDPVYTPMGVFITAWARYTTITAAQACYDRIIYCDTDSIHLTGTEIPDVIKDIVDPKKLGY |
| | WAHESTFKRAKYLRQKTYIQDIYMKEVDGKLVEGSPDDYTDIKFSVKCAGMTDKIKKEVTFENF |
| | KVGFSRKMKPKPVQVPGGVVLVDDTFTIKSGGSKRTADGSEFESPKKKRKV (SEQ ID |
| | NO: 159) |
| B103 | MPRKMFSCDFETTTKLDDCRVWAYGYMEIGNLDNYKIGNSLDEFMQWVMEIQADLYFHNLK |
| (H73R, | FDGAFIVNWLERHGFKWSNEGLPNTYNTIISKMGQWYMIDICFGYKGKRKLHTVIYDSLKKLPF |
| A147K, | PVKKIAKDFQLPLLKGDIDYHKERPVGHEITPEEYEYIKNDIEIIARALDIQFKQGLDRMTAGSDSL |
| R221Y, | KGFKDILSTKKFNKVFPKLSLPMDKEIRYAYRGGFTWLNDKYKEKEIGEGMVFDVNSLYPSQMY |
| A318G, | SRPLPYGAPIVFQGKYEKDEQYPLYIQRIRFEFELKEGYIPTIQIKKNPFFKGNEYLKNSGGEPVELY |
| M339L, | LTNVDLELIQEHYELYNVEYIDGFKFREKTGLFKDFIDKWTYVKTHEEGAKKQLAKLMLNSLYGK |
| E359D, | FASNPDVTGKVPYLKEDGSLGFRVGDEEYKDPVYTPMGVFITAWARFTTITAAQACYDRIIYCD |
| K372E, | TDSIHLTGTEVPEIIKDIVDPKKLGYWAHESTFKRAKYLRQKTYIQDIYMKEVDGKLVECSPDEAT |
| F383L, | TTKFSVKCAGMTDTIKKKVTFDNFKVGFSSKGKPKPVQVNGGVVLVDSVFTIKSGGSSGGSKRT |
| D384N, | ADGSEFESPKKKRKVSGGSSGGSGEIAALEAKIAALKAKNAALKAEIAALEAGY (SEQ ID |
| A503M, | NO: 160) |
| i511V, | |
| R544K, | |
| T550K)- | |
| NLS- | |
| CC(N5) | |
| PCV2- | MSPSKKNGRSGPQPHKRWVFTLNNPSEDERKKIRDLPISLFDYFIVGEEGNEEGRTPHLQGFAN |
| nCas9 | FVKKQTFNKVKWYLGARCHIEKAKGTDQQNKEYCSKEGNLLMECGAPRSQGQRSGGSSGSET |
| (H840A)- | PGTSESATPESSGGSDKKYSIGLDIGTNSVGWAVITDEYKVPSKKFKVLGNTDRHSIKKNLIGALL |
| B103 | FDSGETAEATRLKRTARRRYTRRKNRICYLQEIFSNEMAKVDDSFFHRLEESFLVEEDKKHERHPI |
| (H73R, | FGNIVDEVAYHEKYPTIYHLRKKLVDSTDKADLRLIYLALAHMIKFRGHFLIEGDLNPDNSDVDKL |
| A147K, | FIQLVQTYNQLFEENPINASGVDAKAILSARLSKSRKLENLIAQLPGEKKNGLFGNLIALSLGLTPN |
| R221Y, | FKSNFDLAEDAKLQLSKDTYDDDLDNLLAQIGDQYADLFLAAKNLSDAILLSDILRVNTEITKAPL |
| A318G, | SASMIKRYDEHHQDLTLLKALVRQQLPEKYKEIFFDQSKNGYAGYIDGGASQEEFYKFIKPILEK |
| M339L, | MDGTEELLVKLKREDLLRKQRTFDNGSIPHQIHLGELHAILRRQEDFYPFLKDNREKIEKILTFRIP |
| E359D, | YYVGPLARGNSRFAWMTRKSEETITPWNFEEVVDKGASAQSFIERMTNFDKNLPNEKVLPKHS |
| K372E, | LLYEYFTVYNELTKVKYVTEGMRKPAFLSGEQKKAIVDLLFKTNRKVTVKQLKEDYFKKIECFDSV |
| F383L, | EISGVEDRFNASLGTYHDLLKIIKDKDFLDNEENEDILEDIVLTLTLFEDREMIEERLKTYAHLFDDK |
| D384N, | VMKQLKRRRYTGWGRLSRKLINGIRDKQSGKTILDFLKSDGFANRNFMQLIHDDSLTFKEDIQK |
| A503M, | AQVSGQGDSLHEHIANLAGSPAIKKGILQTVKVVDELVKVMGRHKPENIVIEMARENQTTQKG |
| I511V, | QKNSRERMKRIEEGIKELGSQILKEHPVENTQLQNEKLYLYYLQNGRDMYVDQELDINRLSDYD |
| R544K, | VDAIVPQSFLKDDSIDNKVLTRSDKNRGKSDNVPSEEVVKKMKNYWRQLLNAKLITQRKFDNL |
| T550K)- | TKAERGGLSELDKAGFIKRQLVETRQITKHVAQILDSRMNTKYDENDKLIREVKVITLKSKLVSDF |
| NLS | RKDFQFYKVREINNYHHAHDAYLNAVVGTALIKKYPKLESEFVYGDYKVYDVRKMIAKSEQEIG |
| | KATAKYFFYSNIMNFFKTEITLANGEIRKRPLIETNGETGEIVWDKGRDFATVRKVLSMPQVNIV |
| | KKTEVQTGGFSKESILPKRNSDKLIARKKDWDPKKYGGFDSPTVAYSVLVVAKVEKGKSKKLKSV |
| | KELLGITIMERSSFEKNPIDFLEAKGYKEVKKDLIIKLPKYSLFELENGRKRMLASAGELQKGNELA |
| | LPSKYVNFLYLASHYEKLKGSPEDNEQKQLFVEQHKHYLDEIIEQISEFSKRVILADANLDKVLSAY |
| | NKHRDKPIREQAENIIHLFTLTNLGAPAAFKYFDTTIDRKRYTSTKEVLDATLIHQSITGLYETRIDL |
| | SQLGGDSGGSSGGSKRTADGSEFESPKKKRKVSGGSSGGSPRKMFSCDFETTTKLDDCRVWAY |
| | GYMEIGNLDNYKIGNSLDEFMQWVMEIQADLYFHNLKFDGAFIVNWLERHGFKWSNEGLPN |
| | TYNTIISKMGQWYMIDICFGYKGKRKLHTVIYDSLKKLPFPVKKIAKDFQLPLLKGDIDYHKERPV |
| | GHEITPEEYEYIKNDIEIIARALDIQFKQGLDRMTAGSDSLKGFKDILSTKKFNKVFPKLSLPMDKE |
| | IRYAYRGGFTWLNDKYKEKEIGEGMVFDVNSLYPSQMYSRPLPYGAPIVFQGKYEKDEQYPLYI |
| | QRIRFEFELKEGYIPTIQIKKNPFFKGNEYLKNSGGEPVELYLTNVDLELIQEHYELYNVEYIDGFKF |
| | REKTGLFKDFIDKWTYVKTHEEGAKKQLAKLMLNSLYGKFASNPDVTGKVPYLKEDGSLGFRVG |
| | DEEYKDPVYTPMGVFITAWARFTTITAAQACYDRIIYCDTDSIHLTGTEVPEIIKDIVDPKKLGYW |
| | AHESTFKRAKYLRQKTYIQDIYMKEVDGKLVECSPDEATTTKFSVKCAGMTDTIKKKVTFDNFKV |
| | GFSSKGKPKPVQVNGGVVLVDSVFTIKSGGSKRTADGSEFESPKKKRKV (SEQ ID NO: 161) |
| Phi29 | MKHMPRKRYSCDFETTTKVEDCRVWAYGYMNIEDHSEYKIGNSLDEFMAWALKVQADLYFH |
| (M8R, V51A, | NLKFDGAFIINWLERNGFKWSADGLPNTYNTIISRTGQWYMIDICLGYKGKRKIHTVIYDSLKKL |
| M97T, | PFPVKKIAKDFKLTVLKGDIDYHKERPVGYKITPEEYAYIKNDIQIIAEALLIQFKQGLDRMTAGSD |
| G197D, | SLKDFKDIITTKKFKKVFPTLSLGLDKKVRYAYRGGFTWLNDRFKEKEIGEGMVFDVNSLYPAQM |
| E221K, | YSRLLPYGEPIVFEGKYVWDEDYPLHIQHIRCEFELKEGYIPTIQIKRSRFYKGNEYLKSSGGEIADL |
| Q497P, | WLSNVDLELMKEHYDLYNVEYISGLKFKATTGLFKDFIDKWTYIKTTSEGAIKQLAKLMLNSLYG |
| K512E, | KFASNPDVTGKVPYLKENGALGFRLGEEETKDPVYTPMGVFITAWARYTTITAAQACYDRIIYC |
| F526L)- | DTDSIHLTGTEIPDVIKDIVDPKKLGYWAHESTFKRAKYLRPKTYIQDIYMKEVDGELVEGSPDDY |
| NLS- | TDIKLSVKCAGMTDKIKKEVTFENFKVGFSRKMKPKPVQVPGGVVLVDDTFTIKSGGSSGGSKR |
| CC(N5) | TADGSEFESPKKKRKVSGGSSGGSGEIAALEAKIAALKAKNAALKAEIAALEAGY (SEQ ID |
| | NO: 162) |
| PCV2- | MSPSKKNGRSGPQPHKRWVFTLNNPSEDERKKIRDLPISLFDYFIVGEEGNEEGRTPHLQGFAN |
| nCas9 | FVKKQTFNKVKWYLGARCHIEKAKGTDQQNKEYCSKEGNLLMECGAPRSQGQRSGGSSGSET |
| (H840A)- | PGTSESATPESSGGSDKKYSIGLDIGTNSVGWAVITDEYKVPSKKFKVLGNTDRHSIKKNLIGALL |
| Phi29 | FDSGETAEATRLKRTARRRYTRRKNRICYLQEIFSNEMAKVDDSFFHRLEESFLVEEDKKHERHPI |
| (M8R, V51A, | FGNIVDEVAYHEKYPTIYHLRKKLVDSTDKADLRLIYLALAHMIKFRGHFLIEGDLNPDNSDVDKL |
| M97T, | FIQLVQTYNQLFEENPINASGVDAKAILSARLSKSRKLENLIAQLPGEKKNGLFGNLIALSLGLTPN |
| G197D, | FKSNFDLAEDAKLQLSKDTYDDDLDNLLAQIGDQYADLFLAAKNLSDAILLSDILRVNTEITKAPL |
| E221K, | SASMIKRYDEHHQDLTLLKALVRQQLPEKYKEIFFDQSKNGYAGYIDGGASQEEFYKFIKPILEK |
| Q497P, | MDGTEELLVKLKREDLLRKQRTFDNGSIPHQIHLGELHAILRRQEDFYPFLKDNREKIEKILTFRIP |
| K512E, | YYVGPLARGNSRFAWMTRKSEETITPWNFEEVVDKGASAQSFIERMTNFDKNLPNEKVLPKHS |
| F526L)- | LLYEYFTVYNELTKVKYVTEGMRKPAFLSGEQKKAIVDLLFKTNRKVTVKQLKEDYFKKIECFDSV |
| NLS | EISGVEDRFNASLGTYHDLLKIIKDKDFLDNEENEDILEDIVLTLTLFEDREMIEERLKTYAHLFDDK |
| | VMKQLKRRRYTGWGRLSRKLINGIRDKQSGKTILDFLKSDGFANRNFMQLIHDDSLTFKEDIQK |
| | AQVSGQGDSLHEHIANLAGSPAIKKGILQTVKVVDELVKVMGRHKPENIVIEMARENQTTQKG |
| | QKNSRERMKRIEEGIKELGSQILKEHPVENTQLQNEKLYLYYLQNGRDMYVDQELDINRLSDYD |
| | VDAIVPQSFLKDDSIDNKVLTRSDKNRGKSDNVPSEEVVKKMKNYWRQLLNAKLITQRKFDNL |
| | TKAERGGLSELDKAGFIKRQLVETRQITKHVAQILDSRMNTKYDENDKLIREVKVITLKSKLVSDF |
| | RKDFQFYKVREINNYHHAHDAYLNAVVGTALIKKYPKLESEFVYGDYKVYDVRKMIAKSEQEIG |
| | KATAKYFFYSNIMNFFKTEITLANGEIRKRPLIETNGETGEIVWDKGRDFATVRKVLSMPQVNIV |
| | KKTEVQTGGFSKESILPKRNSDKLIARKKDWDPKKYGGFDSPTVAYSVLVVAKVEKGKSKKLKSV |
| | KELLGITIMERSSFEKNPIDFLEAKGYKEVKKDLIIKLPKYSLFELENGRKRMLASAGELQKGNELA |
| | LPSKYVNFLYLASHYEKLKGSPEDNEQKQLFVEQHKHYLDEIIEQISEFSKRVILADANLDKVLSAY |
| | NKHRDKPIREQAENIIHLFTLTNLGAPAAFKYFDTTIDRKRYTSTKEVLDATLIHQSITGLYETRIDL |
| | SQLGGDSGGSSGGSKRTADGSEFESPKKKRKVSGGSSGGSKHMPRKRYSCDFETTTKVEDCRV |
| | WAYGYMNIEDHSEYKIGNSLDEFMAWALKVQADLYFHNLKFDGAFIINWLERNGFKWSADG |
| | LPNTYNTIISRTGQWYMIDICLGYKGKRKIHTVIYDSLKKLPFPVKKIAKDFKLTVLKGDIDYHKER |
| | PVGYKITPEEYAYIKNAIQIIAEALLIQFKQGLDRMTAGSDSLKDFKDIITTKKFKKVFPTLSLGLDK |
| | KVRYAYRGGFTWLNDRFKEKEIGEGMVFDVNSLYPAQMYSRLLPYGEPIVFEGKYVWDEDYPL |
| | HIQHIRCEFELKEGYIPTIQIKRSRFYKGNEYLKSSGGEIADLWLSNVDLELMKEHYDLYNVEYISG |
| | LKFKATTGLFKDFIDKWTYIKTTSEGAIKQLAKLMLNSLYGKFASNPDVTGKVPYLKENGALGFRL |
| | GEEETKDPVYTPMGVFITAWARYTTITAAQACYDRIIYCDTDSIHLTGTEIPDVIKDIVDPKKLGY |
| | WAHESTFKRAKYLRPKTYIQDIYMKEVDGELVEGSPDDYTDIKLSVKCAGMTDKIKKEVTFENFK |
| | VGFSRKMKPKPVQVPGGVVLVDDTFTIKSGGSKRTADGSEFESPKKKRKV (SEQ ID |
| | NO: 163) |
| PCV2- | MSPSKKNGRSGPQPHKRWVFTLNNPSEDERKKIRDLPISLFDYFIVGEEGNEEGRTPHLQGFAN |
| nCas9 | FVKKQTFNKVKWYLGARCHIEKAKGTDQQNKEYCSKEGNLLMECGAPRSQGQRSGGSSGSET |
| (H840A)- | PGTSESATPESSGGSDKKYSIGLDIGTNSVGWAVITDEYKVPSKKFKVLGNTDRHSIKKNLIGALL |
| Phi29 | FDSGETAEATRLKRTARRRYTRRKNRICYLQEIFSNEMAKVDDSFFHRLEESFLVEEDKKHERHPI |
| (D169A | FGNIVDEVAYHEKYPTIYHLRKKLVDSTDKADLRLIYLALAHMIKFRGHFLIEGDLNPDNSDVDKL |
| M8R, V51A, | FIQLVQTYNQLFEENPINASGVDAKAILSARLSKSRKLENLIAQLPGEKKNGLFGNLIALSLGLTPN |
| M97T, | FKSNFDLAEDAKLQLSKDTYDDDLDNLLAQIGDQYADLFLAAKNLSDAILLSDILRVNTEITKAPL |
| G197D, | SASMIKRYDEHHQDLTLLKALVRQQLPEKYKEIFFDQSKNGYAGYIDGGASQEEFYKFIKPILEK |
| E221K, | MDGTEELLVKLKREDLLRKQRTFDNGSIPHQIHLGELHAILRRQEDFYPFLKDNREKIEKILTFRIP |
| Q497P, | YYVGPLARGNSRFAWMTRKSEETITPWNFEEVVDKGASAQSFIERMTNFDKNLPNEKVLPKHS |
| K512E, | LLYEYFTVYNELTKVKYVTEGMRKPAFLSGEQKKAIVDLLFKTNRKVTVKQLKEDYFKKIECFDSV |
| F526L)- | EISGVEDRFNASLGTYHDLLKIIKDKDFLDNEENEDILEDIVLTLTLFEDREMIEERLKTYAHLFDDK |
| NLS | VMKQLKRRRYTGWGRLSRKLINGIRDKQSGKTILDFLKSDGFANRNFMQLIHDDSLTFKEDIQK |
| | AQVSGQGDSLHEHIANLAGSPAIKKGILQTVKVVDELVKVMGRHKPENIVIEMARENQTTQKG |
| | QKNSRERMKRIEEGIKELGSQILKEHPVENTQLQNEKLYLYYLQNGRDMYVDQELDINRLSDYD |
| | VDAIVPQSFLKDDSIDNKVLTRSDKNRGKSDNVPSEEVVKKMKNYWRQLLNAKLITQRKFDNL |
| | TKAERGGLSELDKAGFIKRQLVETRQITKHVAQILDSRMNTKYDENDKLIREVKVITLKSKLVSDF |
| | RKDFQFYKVREINNYHHAHDAYLNAVVGTALIKKYPKLESEFVYGDYKVYDVRKMIAKSEQEIG |
| | KATAKYFFYSNIMNFFKTEITLANGEIRKRPLIETNGETGEIVWDKGRDFATVRKVLSMPQVNIV |
| | KKTEVQTGGFSKESILPKRNSDKLIARKKDWDPKKYGGFDSPTVAYSVLVVAKVEKGKSKKLKSV |
| | KELLGITIMERSSFEKNPIDFLEAKGYKEVKKDLIIKLPKYSLFELENGRKRMLASAGELQKGNELA |
| | LPSKYVNFLYLASHYEKLKGSPEDNEQKQLFVEQHKHYLDEIIEQISEFSKRVILADANLDKVLSAY |
| | NKHRDKPIREQAENIIHLFTLTNLGAPAAFKYFDTTIDRKRYTSTKEVLDATLIHQSITGLYETRIDL |
| | SQLGGDSGGSSGGSKRTADGSEFESPKKKRKVSGGSSGGSKHMPRKRYSCDFETTTKVEDCRV |
| | WAYGYMNIEDHSEYKIGNSLDEFMAWALKVQADLYFHNLKFDGAFIINWLERNGFKWSADG |
| | LPNTYNTIISRTGQWYMIDICLGYKGKRKIHTVIYDSLKKLPFPVKKIAKDFKLTVLKGDIDYHKER |
| | PVGYKITPEEYAYIKNAIQIIAEALLIQFKQGLDRMTAGSDSLKDFKDIITTKKFKKVFPTLSLGLDK |
| | KVRYAYRGGFTWLNDRFKEKEIGEGMVFDVNSLYPAQMYSRLLPYGEPIVFEGKYVWDEDYPL |
| | HIQHIRCEFELKEGYIPTIQIKRSRFYKGNEYLKSSGGEIADLWLSNVDLELMKEHYDLYNVEYISG |
| | LKFKATTGLFKDFIDKWTYIKTTSEGAIKQLAKLMLNSLYGKFASNPDVTGKVPYLKENGALGFRL |
| | GEEETKDPVYTPMGVFITAWARYTTITAAQACYDRIIYCDTDSIHLTGTEIPDVIKDIVDPKKLGY |
| | WAHESTFKRAKYLRPKTYIQDIYMKEVDGELVEGSPDDYTDIKLSVKCAGMTDKIKKEVTFENFK |
| | VGFSRKMKPKPVQVPGGVVLVDDTFTIKSGGSKRTADGSEFESPKKKRKV (SEQ ID |
| | NO: 163) |
| Phi29 | MKHMPRKRYSCDFETTTKVEDCRVWAYGYMNIEDHSEYKIGNSLDEFMAWALKVQADLYFH |
| (M8R, | NLKFDGAFIINWLERNGFKWSADGLPNTYNTIISRTGQWYMIDICLGYKGKRKIHTVIYDSLKKL |
| V51A, | PFPVKKIAKDFKLTVLKGDIDYHKERPVGYKITPEEYAYIKNDIQIIAEALLIQFKQGLDRMTAGSD |
| M97T, | SLKDFKDIITTKKFKKVFPTLSLGLDKKVRYAYRGGFTWLNDRFKEKEIGEGMVFDVNSLYPAQM |
| G197D, | YSRLLPYGEPIVFEGKYVWDEDYPLHIQHIRCEFELKEGYIPTIQIKRSRFYKGNEYLKSSGGEIADL |
| E221K, | WLSNVDLELMKEHYDLYNVEYISGLKFKATTGLFKDFIDKWTYIKTTSEGAIKQLAKLMLNSLYG |
| Q497P, | KFASNPDVTGKVPYLKENGALGFRLGEEETKDPVYTPMGVFITAWARYTTITAAQACYDRIIYC |
| K512E, | DTDSIHLTGTEIPDVIKDIVDPKKLGYWAHESTFKRAKYLRPKTYIQDIYMKEVDGELVEGSPDDY |
| F526L)- | TDIKLSVKCAGMTDKIKKEVTFENFKVGFSRKMKPKPVQVPGGVVLVDDTFTIKSGGSSGGSKR |
| NLS | TADGSEFESPKKKRKV (SEQ ID NO: 165) |
| pCMV- | MSPEDHTRPGTIPSKEGSGCRRWCFTLNNPTDGEIEFVRSLGPDEFYYAIVGREKGEQGTPHLQGYFH |
| T7- | FKNKKRLSALKKMLPRGHFERAKGSDADNEKYCSKEGDVILTLGIVARDGHRSGGSSGSETPGTSESA |
| BDFV- | TPESSGGSDKKYSIGLDIGTNSVGWAVITDEYKVPSKKFKVLGNTDRHSIKKNLIGALLFDSGETAEA |
| XTEN24- | TRLKRTARRRYTRRKNRICYLQEIFSNEMAKVDDSFFHRLEESFLVEEDKKHERHPIFGNIVDEVAYH |
| nCas9 | EKYPTIYHLRKKLVDSTDKADLRLIYLALAHMIKFRGHFLIEGDLNPDNSDVDKLFIQLVQTYNQLFE |
| (H840A)- | ENPINASGVDAKAILSARLSKSRRLENLIAQLPGEKKNGLFGNLIALSLGLTPNFKSNFDLAEDAKLQ |
| SGGSx2 | LSKDTYDDDLDNLLAQIGDQYADLFLAAKNLSDAILLSDILRVNTEITKAPLSASMIKRYDEHHQDLT |
| (SEQ ID | LLKALVRQQLPEKYKEIFFDQSKNGYAGYIDGGASQEEFYKFIKPILEKMDGTEELLVKLNREDLLRK |
| NO: | QRTFDNGSIPHQIHLGELHAILRRQEDFYPFLKDNREKIEKILTFRIPYYVGPLARGNSRFAWMTRKS |
| 572)- | EETITPWNFEEVVDKGASAQSFIERMTNFDKNLPNEKVLPKHSLLYEYFTVYNELTKVKYVTEGMRKP |
| SV40NLS- | AFLSGEQKKAIVDLLFKTNRKVTVKQLKEDYFKKIECFDSVEISGVEDRFNASLGTYHDLLKIIKDKD |
| SGGSx2 | FLDNEENEDILEDIVLTLTLFEDREMIEERLKTYAHLFDDKVMKQLKRRRYTGWGRLSRKLINGIRDK |
| (SEQ ID | QSGKTILDFLKSDGFANRNFMQLIHDDSLTFKEDIQKAQVSGQGDSLHEHIANLAGSPAIKKGILQTV |
| NO: | KVVDELVKVMGRHKPENIVIEMARENQTTQKGQKNSRERMKRIEEGIKELGSQILKEHPVENTQLQNE |
| 572)- | KLYLYYLQNGRDMYVDQELDINRLSDYDVDAIVPQSFLKDDSIDNKVLTRSDKNRGKSDNVPSEEVVK |
| Klenow | KMKNYWRQLLNAKLITQRKFDNLTKAERGGLSELDKAGFIKRQLVETRQITKHVAQILDSRMNTKYDE |
| (E.coli)- | NDKLIREVKVITLKSKLVSDFRKDFQFYKVREINNYHHAHDAYLNAVVGTALIKKYPKLESEFVYGDY |
| SV40NLS | KVYDVRKMIAKSEQEIGKATAKYFFYSNIMNFFKTEITLANGEIRKRPLIETNGETGEIVWDKGRDFA |
| | TVRKVLSMPQVNIVKKTEVQTGGFSKESILPKRNSDKLIARKKDWDPKKYGGFDSPTVAYSVLVVAKV |
| | EKGKSKKLKSVKELLGITIMERSSFEKNPIDFLEAKGYKEVKKDLIIKLPKYSLFELENGRKRMLASA |
| | GELQKGNELALPSKYVNFLYLASHYEKLKGSPEDNEQKQLFVEQHKHYLDEIIEQISEFSKRVILADA |
| | NLDKVLSAYNKHRDKPIREQAENIIHLFTLTNLGAPAAFKYFDTTIDRKRYTSTKEVLDATLIHQSIT |
| | GLYETRIDLSQLGGDSGGSSGGSKRTADGSEFESPKKKRKVSGGSSGGSVISYDNYVTILDEETLKAW |
| | IAKLEKAPVFAFATATDSLDNISANLVGLSFAIEPGVAAYIPVAHDYLDAPDQISRERALELLKPLLE |
| | DEKALKVGQNLKYDRGILANYGIELRGIAFDTMLESYILNSVAGRHDMDSLAERWLKHKTITFEEIAG |
| | KGKNQLTFNQIALEEAGRYAAEDADVTLQLHLKMWPDLQKHKGPLNVFENIEMPLVPVLSRIERNGVK |
| | IDPKVLHNHSEELTLRLAELEKKAHEIAGEEFNLSSTKQLQTILFEKQGIKPLKKTPGGAPSTSEEVL |
| | EELALDYPLPKVILEYRGLAKLKSTYTDKLPLMINPKTGRVHTSYHQAVTATGRLSSTDPNLQNIPVR |
| | NEEGRRIRQAFIAPEDYVIVSADYSQIELRIMAHLSRDKGLLTAFAEGKDIHRATAAEVFGLPLETVT |
| | SEQRRSAKAINFGLIYGMSAFGLARQLNIPRKEAQKYMDLYFERYPGVLEYMERTRAQAKEQGYVETL |
| | DGRRLYLPDIKSSNGARRAAAERAAINAPMQGTAADIIKRAMIAVDAWLQAEQPRVRMIMQVHDELVF |
| | EVHKDDVDAVAKQIHQLMENCTRLDVPLLVEVGSGENWDQAHSGGSKRTADGSEFESPKKKRKV |
| | (SEQ ID NO: 166) |
| pCMV- | MAPCKPGSNPPKGRVSAAEGGARREATRRPPREAAAKRWCFTLNNPTEEEIKSLETWLVSDFHYAIVG |
| T7- | KEVGEQGTPHLQGFVHLKQKKRLPQLKQLFKRAHWEKARGSDEDNEKYCSKEGNVLLTLGIPAKGNRS |
| CoCV- | GGSSGSETPGTSESATPESSGGSDKKYSIGLDIGTNSVGWAVITDEYKVPSKKFKVLGNTDRHSIKKN |
| XTEN24- | LIGALLFDSGETAEATRLKRTARRRYTRRKNRICYLQEIFSNEMAKVDDSFFHRLEESFLVEEDKKHE |
| nCas9 | RHPIFGNIVDEVAYHEKYPTIYHLRKKLVDSTDKADLRLIYLALAHMIKFRGHFLIEGDLNPDNSDVD |
| (H840A)- | KLFIQLVQTYNQLFEENPINASGVDAKAILSARLSKSRRLENLIAQLPGEKKNGLFGNLIALSLGLTP |
| SGGSx2 | NFKSNFDLAEDAKLQLSKDTYDDDLDNLLAQIGDQYADLFLAAKNLSDAILLSDILRVNTEITKAPLS |
| (SEQ ID | ASMIKRYDEHHQDLTLLKALVRQQLPEKYKEIFFDQSKNGYAGYIDGGASQEEFYKFIKPILEKMDGT |
| NO: | EELLVKLNREDLLRKQRTFDNGSIPHQIHLGELHAILRRQEDFYPFLKDNREKIEKILTFRIPYYVGP |
| 572)- | LARGNSRFAWMTRKSEETITPWNFEEVVDKGASAQSFIERMTNFDKNLPNEKVLPKHSLLYEYFTVYN |
| SV40NLS- | ELTKVKYVTEGMRKPAFLSGEQKKAIVDLLFKTNRKVTVKQLKEDYFKKIECFDSVEISGVEDRFNAS |
| SGGSx2 | LGTYHDLLKIIKDKDFLDNEENEDILEDIVLTLTLFEDREMIEERLKTYAHLFDDKVMKQLKRRRYTG |
| (SEQ ID | WGRLSRKLINGIRDKQSGKTILDFLKSDGFANRNFMQLIHDDSLTFKEDIQKAQVSGQGDSLHEHIAN |
| NO: | LAGSPAIKKGILQTVKVVDELVKVMGRHKPENIVIEMARENQTTQKGQKNSRERMKRIEEGIKELGSQ |
| 572)- | ILKEHPVENTQLQNEKLYLYYLQNGRDMYVDQELDINRLSDYDVDAIVPQSFLKDDSIDNKVLTRSDK |
| Klenow | NRGKSDNVPSEEVVKKMKNYWRQLLNAKLITQRKFDNLTKAERGGLSELDKAGFIKRQLVETRQITKH |
| (E.coli)- | VAQILDSRMNTKYDENDKLIREVKVITLKSKLVSDFRKDFQFYKVREINNYHHAHDAYLNAVVGTALI |
| SV40NLS | KKYPKLESEFVYGDYKVYDVRKMIAKSEQEIGKATAKYFFYSNIMNFFKTEITLANGEIRKRPLIETN |
| | GETGEIVWDKGRDFATVRKVLSMPQVNIVKKTEVQTGGFSKESILPKRNSDKLIARKKDWDPKKYGGF |
| | DSPTVAYSVLVVAKVEKGKSKKLKSVKELLGITIMERSSFEKNPIDFLEAKGYKEVKKDLIIKLPKYS |
| | LFELENGRKRMLASAGELQKGNELALPSKYVNFLYLASHYEKLKGSPEDNEQKQLFVEQHKHYLDEII |
| | EQISEFSKRVILADANLDKVLSAYNKHRDKPIREQAENIIHLFTLTNLGAPAAFKYFDTTIDRKRYTS |
| | TKEVLDATLIHQSITGLYETRIDLSQLGGDSGGSSGGSKRTADGSEFESPKKKRKVSGGSSGGSVISY |
| | DNYVTILDEETLKAWIAKLEKAPVFAFATATDSLDNISANLVGLSFAIEPGVAAYIPVAHDYLDAPDQ |
| | ISRERALELLKPLLEDEKALKVGQNLKYDRGILANYGIELRGIAFDTMLESYILNSVAGRHDMDSLAE |
| | RWLKHKTITFEEIAGKGKNQLTFNQIALEEAGRYAAEDADVTLQLHLKMWPDLQKHKGPLNVFENIEM |
| | PLVPVLSRIERNGVKIDPKVLHNHSEELTLRLAELEKKAHEIAGEEFNLSSTKQLQTILFEKQGIKPL |
| | KKTPGGAPSTSEEVLEELALDYPLPKVILEYRGLAKLKSTYTDKLPLMINPKTGRVHTSYHQAVTATG |
| | RLSSTDPNLQNIPVRNEEGRRIRQAFIAPEDYVIVSADYSQIELRIMAHLSRDKGLLTAFAEGKDIHR |
| | ATAAEVFGLPLETVTSEQRRSAKAINFGLIYGMSAFGLARQLNIPRKEAQKYMDLYFERYPGVLEYME |
| | RTRAQAKEQGYVETLDGRRLYLPDIKSSNGARRAAAERAAINAPMQGTAADIIKRAMIAVDAWLQAEQ |
| | PRVRMIMQVHDELVFEVHKDDVDAVAKQIHQLMENCTRLDVPLLVEVGSGENWDQAHSGGSKRTADGS |
| | EFESPKKKRKV (SEQ ID NO: 167) |
| pCMV- | MPKQARESPCKRWCFTLNNPTEEEIERVKNLSPSEYHYAIVGKEKGEQGTPHLQGFLHLKKKQRLKQM |
| T7- | KELIPRAHFERARGSDEDNEQYCGKEGDVILTIGVPSKGNRSGGSSGSETPGTSESATPESSGGSDKK |
| FICV- | YSIGLDIGTNSVGWAVITDEYKVPSKKFKVLGNTDRHSIKKNLIGALLFDSGETAEATRLKRTARRRY |
| XTEN24- | TRRKNRICYLQEIFSNEMAKVDDSFFHRLEESFLVEEDKKHERHPIFGNIVDEVAYHEKYPTIYHLRK |
| nCas9 | KLVDSTDKADLRLIYLALAHMIKFRGHFLIEGDLNPDNSDVDKLFIQLVQTYNQLFEENPINASGVDA |
| (H840A)- | KAILSARLSKSRRLENLIAQLPGEKKNGLFGNLIALSLGLTPNFKSNFDLAEDAKLQLSKDTYDDDLD |
| SGGSx2 | NLLAQIGDQYADLFLAAKNLSDAILLSDILRVNTEITKAPLSASMIKRYDEHHQDLTLLKALVRQQLP |
| (SEQ ID | EKYKEIFFDQSKNGYAGYIDGGASQEEFYKFIKPILEKMDGTEELLVKLNREDLLRKQRTFDNGSIPH |
| NO: | QIHLGELHAILRRQEDFYPFLKDNREKIEKILTFRIPYYVGPLARGNSRFAWMTRKSEETITPWNFEE |
| 572)- | VVDKGASAQSFIERMTNFDKNLPNEKVLPKHSLLYEYFTVYNELTKVKYVTEGMRKPAFLSGEQKKAI |
| SV40NLS- | VDLLFKTNRKVTVKQLKEDYFKKIECFDSVEISGVEDRFNASLGTYHDLLKIIKDKDFLDNEENEDIL |
| SGGSx2 | EDIVLTLTLFEDREMIEERLKTYAHLFDDKVMKQLKRRRYTGWGRLSRKLINGIRDKQSGKTILDFLK |
| (SEQ ID | SDGFANRNFMQLIHDDSLTFKEDIQKAQVSGQGDSLHEHIANLAGSPAIKKGILQTVKVVDELVKVMG |
| NO: | RHKPENIVIEMARENQTTQKGQKNSRERMKRIEEGIKELGSQILKEHPVENTQLQNEKLYLYYLQNGR |
| 572)- | DMYVDQELDINRLSDYDVDAIVPQSFLKDDSIDNKVLTRSDKNRGKSDNVPSEEVVKKMKNYWRQLLN |
| Klenow | AKLITQRKFDNLTKAERGGLSELDKAGFIKRQLVETRQITKHVAQILDSRMNTKYDENDKLIREVKVI |
| (E.coli)- | TLKSKLVSDFRKDFQFYKVREINNYHHAHDAYLNAVVGTALIKKYPKLESEFVYGDYKVYDVRKMIAK |
| SV40NLS | SEQEIGKATAKYFFYSNIMNFFKTEITLANGEIRKRPLIETNGETGEIVWDKGRDFATVRKVLSMPQV |
| | NIVKKTEVQTGGFSKESILPKRNSDKLIARKKDWDPKKYGGFDSPTVAYSVLVVAKVEKGKSKKLKSV |
| | KELLGITIMERSSFEKNPIDFLEAKGYKEVKKDLIIKLPKYSLFELENGRKRMLASAGELQKGNELAL |
| | PSKYVNFLYLASHYEKLKGSPEDNEQKQLFVEQHKHYLDEIIEQISEFSKRVILADANLDKVLSAYNK |
| | HRDKPIREQAENIIHLFTLTNLGAPAAFKYFDTTIDRKRYTSTKEVLDATLIHQSITGLYETRIDLSQ |
| | LGGDSGGSSGGSKRTADGSEFESPKKKRKVSGGSSGGSVISYDNYVTILDEETLKAWIAKLEKAPVFA |
| | FATATDSLDNISANLVGLSFAIEPGVAAYIPVAHDYLDAPDQISRERALELLKPLLEDEKALKVGQNL |
| | KYDRGILANYGIELRGIAFDTMLESYILNSVAGRHDMDSLAERWLKHKTITFEEIAGKGKNQLTFNQI |
| | ALEEAGRYAAEDADVTLQLHLKMWPDLQKHKGPLNVFENIEMPLVPVLSRIERNGVKIDPKVLHNHSE |
| | ELTLRLAELEKKAHEIAGEEFNLSSTKQLQTILFEKQGIKPLKKTPGGAPSTSEEVLEELALDYPLPK |
| | VILEYRGLAKLKSTYTDKLPLMINPKTGRVHTSYHQAVTATGRLSSTDPNLQNIPVRNEEGRRIRQAF |
| | IAPEDYVIVSADYSQIELRIMAHLSRDKGLLTAFAEGKDIHRATAAEVFGLPLETVTSEQRRSAKAIN |
| | FGLIYGMSAFGLARQLNIPRKEAQKYMDLYFERYPGVLEYMERTRAQAKEQGYVETLDGRRLYLPDIK |
| | SSNGARRAAAERAAINAPMQGTAADIIKRAMIAVDAWLQAEQPRVRMIMQVHDELVFEVHKDDVDAVA |
| | KQIHQLMENCTRLDVPLLVEVGSGENWDQAHSGGSKRTADGSEFESPKKKRKV (SEQ ID |
| | NO: 168) |
| pCMV- | MRRRPRSGRYLRSIMAARRDSGARRWCFTLNNYTPEEEETARNLIHDADKYAFAIIGKEVGESGTPHL |
| T7- | QGFMHFKQKQRLTALKKLFPRAHFEKARGSDQQNADYCGKDGEILTMIGTPSDNNPSGGSSGSETPGT |
| GuCV- | SESATPESSGGSDKKYSIGLDIGTNSVGWAVITDEYKVPSKKFKVLGNTDRHSIKKNLIGALLFDSGE |
| XTEN24- | TAEATRLKRTARRRYTRRKNRICYLQEIFSNEMAKVDDSFFHRLEESFLVEEDKKHERHPIFGNIVDE |
| nCas9 | VAYHEKYPTIYHLRKKLVDSTDKADLRLIYLALAHMIKFRGHFLIEGDLNPDNSDVDKLFIQLVQTYN |
| (H840A)- | QLFEENPINASGVDAKAILSARLSKSRRLENLIAQLPGEKKNGLFGNLIALSLGLTPNFKSNFDLAED |
| SGGSx2 | AKLQLSKDTYDDDLDNLLAQIGDQYADLFLAAKNLSDAILLSDILRVNTEITKAPLSASMIKRYDEHH |
| (SEQ ID | QDLTLLKALVRQQLPEKYKEIFFDQSKNGYAGYIDGGASQEEFYKFIKPILEKMDGTEELLVKLNRED |
| NO: | LLRKQRTFDNGSIPHQIHLGELHAILRRQEDFYPFLKDNREKIEKILTFRIPYYVGPLARGNSRFAWM |
| 572)- | TRKSEETITPWNFEEVVDKGASAQSFIERMTNFDKNLPNEKVLPKHSLLYEYFTVYNELTKVKYVTEG |
| SV40NLS- | MRKPAFLSGEQKKAIVDLLFKTNRKVTVKQLKEDYFKKIECFDSVEISGVEDRFNASLGTYHDLLKII |
| SGGSx2 | KDKDFLDNEENEDILEDIVLTLTLFEDREMIEERLKTYAHLFDDKVMKQLKRRRYTGWGRLSRKLING |
| (SEQ ID | IRDKQSGKTILDFLKSDGFANRNFMQLIHDDSLTFKEDIQKAQVSGQGDSLHEHIANLAGSPAIKKGI |
| NO: | LQTVKVVDELVKVMGRHKPENIVIEMARENQTTQKGQKNSRERMKRIEEGIKELGSQILKEHPVENTQ |
| 572)- | LQNEKLYLYYLQNGRDMYVDQELDINRLSDYDVDAIVPQSFLKDDSIDNKVLTRSDKNRGKSDNVPSE |
| Klenow | EVVKKMKNYWRQLLNAKLITQRKFDNLTKAERGGLSELDKAGFIKRQLVETRQITKHVAQILDSRMNT |
| (E.coli)- | KYDENDKLIREVKVITLKSKLVSDFRKDFQFYKVREINNYHHAHDAYLNAVVGTALIKKYPKLESEFV |
| SV40NLS | YGDYKVYDVRKMIAKSEQEIGKATAKYFFYSNIMNFFKTEITLANGEIRKRPLIETNGETGEIVWDKG |
| | RDFATVRKVLSMPQVNIVKKTEVQTGGFSKESILPKRNSDKLIARKKDWDPKKYGGFDSPTVAYSVLV |
| | VAKVEKGKSKKLKSVKELLGITIMERSSFEKNPIDFLEAKGYKEVKKDLIIKLPKYSLFELENGRKRM |
| | LASAGELQKGNELALPSKYVNFLYLASHYEKLKGSPEDNEQKQLFVEQHKHYLDEIIEQISEFSKRVI |
| | LADANLDKVLSAYNKHRDKPIREQAENIIHLFTLTNLGAPAAFKYFDTTIDRKRYTSTKEVLDATLIH |
| | QSITGLYETRIDLSQLGGDSGGSSGGSKRTADGSEFESPKKKRKVSGGSSGGSVISYDNYVTILDEET |
| | LKAWIAKLEKAPVFAFATATDSLDNISANLVGLSFAIEPGVAAYIPVAHDYLDAPDQISRERALELLK |
| | PLLEDEKALKVGQNLKYDRGILANYGIELRGIAFDTMLESYILNSVAGRHDMDSLAERWLKHKTITFE |
| | EIAGKGKNQLTFNQIALEEAGRYAAEDADVTLQLHLKMWPDLQKHKGPLNVFENIEMPLVPVLSRIER |
| | NGVKIDPKVLHNHSEELTLRLAELEKKAHEIAGEEFNLSSTKQLQTILFEKQGIKPLKKTPGGAPSTS |
| | EEVLEELALDYPLPKVILEYRGLAKLKSTYTDKLPLMINPKTGRVHTSYHQAVTATGRLSSTDPNLQN |
| | IPVRNEEGRRIRQAFIAPEDYVIVSADYSQIELRIMAHLSRDKGLLTAFAEGKDIHRATAAEVFGLPL |
| | ETVTSEQRRSAKAINFGLIYGMSAFGLARQLNIPRKEAQKYMDLYFERYPGVLEYMERTRAQAKEQGY |
| | VETLDGRRLYLPDIKSSNGARRAAAERAAINAPMQGTAADIIKRAMIAVDAWLQAEQPRVRMIMQVHD |
| | ELVFEVHKDDVDAVAKQIHQLMENCTRLDVPLLVEVGSGENWDQAHSGGSKRTADGSEFESPKKKRKV |
| | (SEQ ID NO: 169) |
| pCMV- | MPSKKSGPQPHKRWVFTLNNPSEEEKNKIRELPISLFDYFVCGEEGLEEGRTPHLQGFANFAKKQTFN |
| T7- | KVKWYFGARCHIEKAKGTDQQNKEYCSKEGHILIECGAPRNQGKRSGGSSGSETPGTSESATPESSGG |
| PCV1- | SDKKYSIGLDIGTNSVGWAVITDEYKVPSKKFKVLGNTDRHSIKKNLIGALLFDSGETAEATRLKRTA |
| XTEN24- | RRRYTRRKNRICYLQEIFSNEMAKVDDSFFHRLEESFLVEEDKKHERHPIFGNIVDEVAYHEKYPTIY |
| nCas9 | HLRKKLVDSTDKADLRLIYLALAHMIKFRGHFLIEGDLNPDNSDVDKLFIQLVQTYNQLFEENPINAS |
| (H840A)- | GVDAKAILSARLSKSRRLENLIAQLPGEKKNGLFGNLIALSLGLTPNFKSNFDLAEDAKLQLSKDTYD |
| SGGSx2 | DDLDNLLAQIGDQYADLFLAAKNLSDAILLSDILRVNTEITKAPLSASMIKRYDEHHQDLTLLKALVR |
| (SEQ ID | QQLPEKYKEIFFDQSKNGYAGYIDGGASQEEFYKFIKPILEKMDGTEELLVKLNREDLLRKQRTFDNG |
| NO: | SIPHQIHLGELHAILRRQEDFYPFLKDNREKIEKILTFRIPYYVGPLARGNSRFAWMTRKSEETITPW |
| 572)- | NFEEVVDKGASAQSFIERMTNFDKNLPNEKVLPKHSLLYEYFTVYNELTKVKYVTEGMRKPAFLSGEQ |
| SV40NLS- | KKAIVDLLFKTNRKVTVKQLKEDYFKKIECFDSVEISGVEDRFNASLGTYHDLLKIIKDKDFLDNEEN |
| SGGSx2 | EDILEDIVLTLTLFEDREMIEERLKTYAHLFDDKVMKQLKRRRYTGWGRLSRKLINGIRDKQSGKTIL |
| (SEQ ID | DFLKSDGFANRNFMQLIHDDSLTFKEDIQKAQVSGQGDSLHEHIANLAGSPAIKKGILQTVKVVDELV |
| NO: | KVMGRHKPENIVIEMARENQTTQKGQKNSRERMKRIEEGIKELGSQILKEHPVENTQLQNEKLYLYYL |
| 572)- | QNGRDMYVDQELDINRLSDYDVDAIVPQSFLKDDSIDNKVLTRSDKNRGKSDNVPSEEVVKKMKNYWR |
| Kleno | QLLNAKLITQRKFDNLTKAERGGLSELDKAGFIKRQLVETRQITKHVAQILDSRMNTKYDENDKLIRE |
| (E.coli)- | VKVITLKSKLVSDFRKDFQFYKVREINNYHHAHDAYLNAVVGTALIKKYPKLESEFVYGDYKVYDVRK |
| SV40NLS | MIAKSEQEIGKATAKYFFYSNIMNFFKTEITLANGEIRKRPLIETNGETGEIVWDKGRDFATVRKVLS |
| | MPQVNIVKKTEVQTGGFSKESILPKRNSDKLIARKKDWDPKKYGGFDSPTVAYSVLVVAKVEKGKSKK |
| | LKSVKELLGITIMERSSFEKNPIDFLEAKGYKEVKKDLIIKLPKYSLFELENGRKRMLASAGELQKGN |
| | ELALPSKYVNFLYLASHYEKLKGSPEDNEQKQLFVEQHKHYLDEIIEQISEFSKRVILADANLDKVLS |
| | AYNKHRDKPIREQAENIIHLFTLTNLGAPAAFKYFDTTIDRKRYTSTKEVLDATLIHQSITGLYETRI |
| | DLSQLGGDSGGSSGGSKRTADGSEFESPKKKRKVSGGSSGGSVISYDNYVTILDEETLKAWIAKLEKA |
| | PVFAFATATDSLDNISANLVGLSFAIEPGVAAYIPVAHDYLDAPDQISRERALELLKPLLEDEKALKV |
| | GQNLKYDRGILANYGIELRGIAFDTMLESYILNSVAGRHDMDSLAERWLKHKTITFEEIAGKGKNQLT |
| | FNQIALEEAGRYAAEDADVTLQLHLKMWPDLQKHKGPLNVFENIEMPLVPVLSRIERNGVKIDPKVLH |
| | NHSEELTLRLAELEKKAHEIAGEEFNLSSTKQLQTILFEKQGIKPLKKTPGGAPSTSEEVLEELALDY |
| | PLPKVILEYRGLAKLKSTYTDKLPLMINPKTGRVHTSYHQAVTATGRLSSTDPNLQNIPVRNEEGRRI |
| | RQAFIAPEDYVIVSADYSQIELRIMAHLSRDKGLLTAFAEGKDIHRATAAEVFGLPLETVTSEQRRSA |
| | KAINFGLIYGMSAFGLARQLNIPRKEAQKYMDLYFERYPGVLEYMERTRAQAKEQGYVETLDGRRLYL |
| | PDIKSSNGARRAAAERAAINAPMQGTAADIIKRAMIAVDAWLQAEQPRVRMIMQVHDELVFEVHKDDV |
| | DAVAKQIHQLMENCTRLDVPLLVEVGSGENWDQAHSGGSKRTADGSEFESPKKKRKV (SEQ ID |
| | NO: 170) |
| pCMV- | MANSTVRRFVFTLNNYTEDQYQKCVEFISTKCKYGIVGKEVGEENGTPHLQGFCNLHKPMRFGTIKKS |
| T7- | IDNAIHIEKANGSDIDNQKYCSKAGNFFETGTPSQQGRRSGGSSGSETPGTSESATPESSGGSDKKYS |
| NG12- | IGLDIGTNSVGWAVITDEYKVPSKKFKVLGNTDRHSIKKNLIGALLFDSGETAEATRLKRTARRRYTR |
| XTEN24- | RKNRICYLQEIFSNEMAKVDDSFFHRLEESFLVEEDKKHERHPIFGNIVDEVAYHEKYPTIYHLRKKL |
| nCas9 | VDSTDKADLRLIYLALAHMIKFRGHFLIEGDLNPDNSDVDKLFIQLVQTYNQLFEENPINASGVDAKA |
| (H840A)- | ILSARLSKSRRLENLIAQLPGEKKNGLFGNLIALSLGLTPNFKSNFDLAEDAKLQLSKDTYDDDLDNL |
| SGGSx2 | LAQIGDQYADLFLAAKNLSDAILLSDILRVNTEITKAPLSASMIKRYDEHHQDLTLLKALVRQQLPEK |
| (SEQ ID | YKEIFFDQSKNGYAGYIDGGASQEEFYKFIKPILEKMDGTEELLVKLNREDLLRKQRTFDNGSIPHQI |
| NO: | HLGELHAILRRQEDFYPFLKDNREKIEKILTFRIPYYVGPLARGNSRFAWMTRKSEETITPWNFEEVV |
| 572)- | DKGASAQSFIERMTNFDKNLPNEKVLPKHSLLYEYFTVYNELTKVKYVTEGMRKPAFLSGEQKKAIVD |
| SV40NLS- | LLFKTNRKVTVKQLKEDYFKKIECFDSVEISGVEDRFNASLGTYHDLLKIIKDKDFLDNEENEDILED |
| SGGSx2 | IVLTLTLFEDREMIEERLKTYAHLFDDKVMKQLKRRRYTGWGRLSRKLINGIRDKQSGKTILDFLKSD |
| (SEQ ID | GFANRNFMQLIHDDSLTFKEDIQKAQVSGQGDSLHEHIANLAGSPAIKKGILQTVKVVDELVKVMGRH |
| NO: | KPENIVIEMARENQTTQKGQKNSRERMKRIEEGIKELGSQILKEHPVENTQLQNEKLYLYYLQNGRDM |
| 572)- | YVDQELDINRLSDYDVDAIVPQSFLKDDSIDNKVLTRSDKNRGKSDNVPSEEVVKKMKNYWRQLLNAK |
| Klenow | LITQRKFDNLTKAERGGLSELDKAGFIKRQLVETRQITKHVAQILDSRMNTKYDENDKLIREVKVITL |
| (E.coli)- | KSKLVSDFRKDFQFYKVREINNYHHAHDAYLNAVVGTALIKKYPKLESEFVYGDYKVYDVRKMIAKSE |
| SV40NLS | QEIGKATAKYFFYSNIMNFFKTEITLANGEIRKRPLIETNGETGEIVWDKGRDFATVRKVLSMPQVNI |
| | VKKTEVQTGGFSKESILPKRNSDKLIARKKDWDPKKYGGFDSPTVAYSVLVVAKVEKGKSKKLKSVKE |
| | LLGITIMERSSFEKNPIDFLEAKGYKEVKKDLIIKLPKYSLFELENGRKRMLASAGELQKGNELALPS |
| | KYVNFLYLASHYEKLKGSPEDNEQKQLFVEQHKHYLDEIIEQISEFSKRVILADANLDKVLSAYNKHR |
| | DKPIREQAENIIHLFTLTNLGAPAAFKYFDTTIDRKRYTSTKEVLDATLIHQSITGLYETRIDLSQLG |
| | GDSGGSSGGSKRTADGSEFESPKKKRKVSGGSSGGSVISYDNYVTILDEETLKAWIAKLEKAPVFAFA |
| | TATDSLDNISANLVGLSFAIEPGVAAYIPVAHDYLDAPDQISRERALELLKPLLEDEKALKVGQNLKY |
| | DRGILANYGIELRGIAFDTMLESYILNSVAGRHDMDSLAERWLKHKTITFEEIAGKGKNQLTFNQIAL |
| | EEAGRYAAEDADVTLQLHLKMWPDLQKHKGPLNVFENIEMPLVPVLSRIERNGVKIDPKVLHNHSEEL |
| | TLRLAELEKKAHEIAGEEFNLSSTKQLQTILFEKQGIKPLKKTPGGAPSTSEEVLEELALDYPLPKVI |
| | LEYRGLAKLKSTYTDKLPLMINPKTGRVHTSYHQAVTATGRLSSTDPNLQNIPVRNEEGRRIRQAFIA |
| | PEDYVIVSADYSQIELRIMAHLSRDKGLLTAFAEGKDIHRATAAEVFGLPLETVTSEQRRSAKAINFG |
| | LIYGMSAFGLARQLNIPRKEAQKYMDLYFERYPGVLEYMERTRAQAKEQGYVETLDGRRLYLPDIKSS |
| | NGARRAAAERAAINAPMQGTAADIIKRAMIAVDAWLQAEQPRVRMIMQVHDELVFEVHKDDVDAVAKQ |
| | IHQLMENCTRLDVPLLVEVGSGENWDQAHSGGSKRTADGSEFESPKKKRKV (SEQ ID NO: 171) |
| pCMV- | MALADVRGMANSTVRRFCFTWNNYSELEYVLCCEFIKKYCCYGIVGKEIAPNTGTHHLQGFCNLEKPM |
| T7- | RFSTIKKRLDSRIHIEKAAGSDSENQTYCSKTGNFFESGTPNQQGRRSGGSSGSETPGTSESATPESS |
| NG14- | GGSDKKYSIGLDIGTNSVGWAVITDEYKVPSKKFKVLGNTDRHSIKKNLIGALLFDSGETAEATRLKR |
| XTEN24- | TARRRYTRRKNRICYLQEIFSNEMAKVDDSFFHRLEESFLVEEDKKHERHPIFGNIVDEVAYHEKYPT |
| nCas9 | IYHLRKKLVDSTDKADLRLIYLALAHMIKFRGHFLIEGDLNPDNSDVDKLFIQLVQTYNQLFEENPIN |
| (H840A)- | ASGVDAKAILSARLSKSRRLENLIAQLPGEKKNGLFGNLIALSLGLTPNFKSNFDLAEDAKLQLSKDT |
| SGGSx2 | YDDDLDNLLAQIGDQYADLFLAAKNLSDAILLSDILRVNTEITKAPLSASMIKRYDEHHQDLTLLKAL |
| (SEQ ID | VRQQLPEKYKEIFFDQSKNGYAGYIDGGASQEEFYKFIKPILEKMDGTEELLVKLNREDLLRKQRTFD |
| NO: | NGSIPHQIHLGELHAILRRQEDFYPFLKDNREKIEKILTFRIPYYVGPLARGNSRFAWMTRKSEETIT |
| 572)- | PWNFEEVVDKGASAQSFIERMTNFDKNLPNEKVLPKHSLLYEYFTVYNELTKVKYVTEGMRKPAFLSG |
| SV40NLS- | EQKKAIVDLLFKTNRKVTVKQLKEDYFKKIECFDSVEISGVEDRFNASLGTYHDLLKIIKDKDFLDNE |
| SGGSx2 | ENEDILEDIVLTLTLFEDREMIEERLKTYAHLFDDKVMKQLKRRRYTGWGRLSRKLINGIRDKQSGKT |
| (SEQ ID | ILDFLKSDGFANRNFMQLIHDDSLTFKEDIQKAQVSGQGDSLHEHIANLAGSPAIKKGILQTVKVVDE |
| NO: | LVKVMGRHKPENIVIEMARENQTTQKGQKNSRERMKRIEEGIKELGSQILKEHPVENTQLQNEKLYLY |
| 572)- | YLQNGRDMYVDQELDINRLSDYDVDAIVPQSFLKDDSIDNKVLTRSDKNRGKSDNVPSEEVVKKMKNY |
| Klenow | WRQLLNAKLITQRKFDNLTKAERGGLSELDKAGFIKRQLVETRQITKHVAQILDSRMNTKYDENDKLI |
| (E.coli)- | REVKVITLKSKLVSDFRKDFQFYKVREINNYHHAHDAYLNAVVGTALIKKYPKLESEFVYGDYKVYDV |
| SV40NLS | RKMIAKSEQEIGKATAKYFFYSNIMNFFKTEITLANGEIRKRPLIETNGETGEIVWDKGRDFATVRKV |
| | LSMPQVNIVKKTEVQTGGFSKESILPKRNSDKLIARKKDWDPKKYGGFDSPTVAYSVLVVAKVEKGKS |
| | KKLKSVKELLGITIMERSSFEKNPIDFLEAKGYKEVKKDLIIKLPKYSLFELENGRKRMLASAGELQK |
| | GNELALPSKYVNFLYLASHYEKLKGSPEDNEQKQLFVEQHKHYLDEIIEQISEFSKRVILADANLDKV |
| | LSAYNKHRDKPIREQAENIIHLFTLTNLGAPAAFKYFDTTIDRKRYTSTKEVLDATLIHQSITGLYET |
| | RIDLSQLGGDSGGSSGGSKRTADGSEFESPKKKRKVSGGSSGGSVISYDNYVTILDEETLKAWIAKLE |
| | KAPVFAFATATDSLDNISANLVGLSFAIEPGVAAYIPVAHDYLDAPDQISRERALELLKPLLEDEKAL |
| | KVGQNLKYDRGILANYGIELRGIAFDTMLESYILNSVAGRHDMDSLAERWLKHKTITFEEIAGKGKNQ |
| | LTFNQIALEEAGRYAAEDADVTLQLHLKMWPDLQKHKGPLNVFENIEMPLVPVLSRIERNGVKIDPKV |
| | LHNHSEELTLRLAELEKKAHEIAGEEFNLSSTKQLQTILFEKQGIKPLKKTPGGAPSTSEEVLEELAL |
| | DYPLPKVILEYRGLAKLKSTYTDKLPLMINPKTGRVHTSYHQAVTATGRLSSTDPNLQNIPVRNEEGR |
| | RIRQAFIAPEDYVIVSADYSQIELRIMAHLSRDKGLLTAFAEGKDIHRATAAEVFGLPLETVTSEQRR |
| | SAKAINFGLIYGMSAFGLARQLNIPRKEAQKYMDLYFERYPGVLEYMERTRAQAKEQGYVETLDGRRL |
| | YLPDIKSSNGARRAAAERAAINAPMQGTAADIIKRAMIAVDAWLQAEQPRVRMIMQVHDELVFEVHKD |
| | DVDAVAKQIHQLMENCTRLDVPLLVEVGSGENWDQAHSGGSKRTADGSEFESPKKKRKV (SEQ ID |
| | NO: 172) |
| pCMV- | MANRTVRRFCFTWNDHPVEAYEKCEKFIEKFCKYGIVGEEYAPTTGMPHLQGFCNLNKPTRFSTIKKH |
| T7- | LDNSIHIEKANGTDEQNQKYCSKSGIFFESGVPNKQGQRSGGSSGSETPGTSESATPESSGGSDKKYS |
| PK5006- | IGLDIGTNSVGWAVITDEYKVPSKKFKVLGNTDRHSIKKNLIGALLFDSGETAEATRLKRTARRRYTR |
| XTEN24- | RKNRICYLQEIFSNEMAKVDDSFFHRLEESFLVEEDKKHERHPIFGNIVDEVAYHEKYPTIYHLRKKL |
| nCas9 | VDSTDKADLRLIYLALAHMIKFRGHFLIEGDLNPDNSDVDKLFIQLVQTYNQLFEENPINASGVDAKA |
| (H840A)- | ILSARLSKSRRLENLIAQLPGEKKNGLFGNLIALSLGLTPNFKSNFDLAEDAKLQLSKDTYDDDLDNL |
| SGGSx2 | LAQIGDQYADLFLAAKNLSDAILLSDILRVNTEITKAPLSASMIKRYDEHHQDLTLLKALVRQQLPEK |
| (SEQ ID | YKEIFFDQSKNGYAGYIDGGASQEEFYKFIKPILEKMDGTEELLVKLNREDLLRKQRTFDNGSIPHQI |
| NO: | HLGELHAILRRQEDFYPFLKDNREKIEKILTFRIPYYVGPLARGNSRFAWMTRKSEETITPWNFEEVV |
| 572)- | DKGASAQSFIERMTNFDKNLPNEKVLPKHSLLYEYFTVYNELTKVKYVTEGMRKPAFLSGEQKKAIVD |
| SV40NLS- | LLFKTNRKVTVKQLKEDYFKKIECFDSVEISGVEDRFNASLGTYHDLLKIIKDKDFLDNEENEDILED |
| SGGSX2 | IVLTLTLFEDREMIEERLKTYAHLFDDKVMKQLKRRRYTGWGRLSRKLINGIRDKQSGKTILDFLKSD |
| (SEQ ID | GFANRNFMQLIHDDSLTFKEDIQKAQVSGQGDSLHEHIANLAGSPAIKKGILQTVKVVDELVKVMGRH |
| NO: | KPENIVIEMARENQTTQKGQKNSRERMKRIEEGIKELGSQILKEHPVENTQLQNEKLYLYYLQNGRDM |
| 572)- | YVDQELDINRLSDYDVDAIVPQSFLKDDSIDNKVLTRSDKNRGKSDNVPSEEVVKKMKNYWRQLLNAK |
| Klenow | LITQRKFDNLTKAERGGLSELDKAGFIKRQLVETRQITKHVAQILDSRMNTKYDENDKLIREVKVITL |
| (E.coli)- | KSKLVSDFRKDFQFYKVREINNYHHAHDAYLNAVVGTALIKKYPKLESEFVYGDYKVYDVRKMIAKSE |
| SV40NLS | QEIGKATAKYFFYSNIMNFFKTEITLANGEIRKRPLIETNGETGEIVWDKGRDFATVRKVLSMPQVNI |
| | VKKTEVQTGGFSKESILPKRNSDKLIARKKDWDPKKYGGFDSPTVAYSVLVVAKVEKGKSKKLKSVKE |
| | LLGITIMERSSFEKNPIDFLEAKGYKEVKKDLIIKLPKYSLFELENGRKRMLASAGELQKGNELALPS |
| | KYVNFLYLASHYEKLKGSPEDNEQKQLFVEQHKHYLDEIIEQISEFSKRVILADANLDKVLSAYNKHR |
| | DKPIREQAENIIHLFTLTNLGAPAAFKYFDTTIDRKRYTSTKEVLDATLIHQSITGLYETRIDLSQLG |
| | GDSGGSSGGSKRTADGSEFESPKKKRKVSGGSSGGSVISYDNYVTILDEETLKAWIAKLEKAPVFAFA |
| | TATDSLDNISANLVGLSFAIEPGVAAYIPVAHDYLDAPDQISRERALELLKPLLEDEKALKVGQNLKY |
| | DRGILANYGIELRGIAFDTMLESYILNSVAGRHDMDSLAERWLKHKTITFEEIAGKGKNQLTFNQIAL |
| | EEAGRYAAEDADVTLQLHLKMWPDLQKHKGPLNVFENIEMPLVPVLSRIERNGVKIDPKVLHNHSEEL |
| | TLRLAELEKKAHEIAGEEFNLSSTKQLQTILFEKQGIKPLKKTPGGAPSTSEEVLEELALDYPLPKVI |
| | LEYRGLAKLKSTYTDKLPLMINPKTGRVHTSYHQAVTATGRLSSTDPNLQNIPVRNEEGRRIRQAFIA |
| | PEDYVIVSADYSQIELRIMAHLSRDKGLLTAFAEGKDIHRATAAEVFGLPLETVTSEQRRSAKAINFG |
| | LIYGMSAFGLARQLNIPRKEAQKYMDLYFERYPGVLEYMERTRAQAKEQGYVETLDGRRLYLPDIKSS |
| | NGARRAAAERAAINAPMQGTAADIIKRAMIAVDAWLQAEQPRVRMIMQVHDELVFEVHKDDVDAVAKQ |
| | IHQLMENCTRLDVPLLVEVGSGENWDQAHSGGSKRTADGSEFESPKKKRKV (SEQ ID NO: 173) |
| pCMV- | MNSTVRRFCFTWNNYTVADTLTVKDYFVKYCKYGIAGEELAPETGTPHLQGFCNLRKPQRFSAIKKHL |
| T7- | SDRIHIEKANGSDEQNQSYCKKAGHWFEQGVPVKQGDRSGGSSGSETPGTSESATPESSGGSDKKYSI |
| PK5034- | GLDIGTNSVGWAVITDEYKVPSKKFKVLGNTDRHSIKKNLIGALLFDSGETAEATRLKRTARRRYTRR |
| XTEN24- | KNRICYLQEIFSNEMAKVDDSFFHRLEESFLVEEDKKHERHPIFGNIVDEVAYHEKYPTIYHLRKKLV |
| nCas9 | DSTDKADLRLIYLALAHMIKFRGHFLIEGDLNPDNSDVDKLFIQLVQTYNQLFEENPINASGVDAKAI |
| (H840A)- | LSARLSKSRRLENLIAQLPGEKKNGLFGNLIALSLGLTPNFKSNFDLAEDAKLQLSKDTYDDDLDNLL |
| SGGSx2 | AQIGDQYADLFLAAKNLSDAILLSDILRVNTEITKAPLSASMIKRYDEHHQDLTLLKALVRQQLPEKY |
| (SEQ ID | KEIFFDQSKNGYAGYIDGGASQEEFYKFIKPILEKMDGTEELLVKLNREDLLRKQRTFDNGSIPHQIH |
| NO: | LGELHAILRRQEDFYPFLKDNREKIEKILTFRIPYYVGPLARGNSRFAWMTRKSEETITPWNFEEVVD |
| 572)- | KGASAQSFIERMTNFDKNLPNEKVLPKHSLLYEYFTVYNELTKVKYVTEGMRKPAFLSGEQKKAIVDL |
| SV40NLS- | LFKTNRKVTVKQLKEDYFKKIECFDSVEISGVEDRFNASLGTYHDLLKIIKDKDFLDNEENEDILEDI |
| SGGSx2 | VLTLTLFEDREMIEERLKTYAHLFDDKVMKQLKRRRYTGWGRLSRKLINGIRDKQSGKTILDFLKSDG |
| (SEQ ID | FANRNFMQLIHDDSLTFKEDIQKAQVSGQGDSLHEHIANLAGSPAIKKGILQTVKVVDELVKVMGRHK |
| NO: | PENIVIEMARENQTTQKGQKNSRERMKRIEEGIKELGSQILKEHPVENTQLQNEKLYLYYLQNGRDMY |
| 572)- | VDQELDINRLSDYDVDAIVPQSFLKDDSIDNKVLTRSDKNRGKSDNVPSEEVVKKMKNYWRQLLNAKL |
| Klenow | ITQRKFDNLTKAERGGLSELDKAGFIKRQLVETRQITKHVAQILDSRMNTKYDENDKLIREVKVITLK |
| (E.coli)- | SKLVSDFRKDFQFYKVREINNYHHAHDAYLNAVVGTALIKKYPKLESEFVYGDYKVYDVRKMIAKSEQ |
| SV40NLS | EIGKATAKYFFYSNIMNFFKTEITLANGEIRKRPLIETNGETGEIVWDKGRDFATVRKVLSMPQVNIV |
| | KKTEVQTGGFSKESILPKRNSDKLIARKKDWDPKKYGGFDSPTVAYSVLVVAKVEKGKSKKLKSVKEL |
| | LGITIMERSSFEKNPIDFLEAKGYKEVKKDLIIKLPKYSLFELENGRKRMLASAGELQKGNELALPSK |
| | YVNFLYLASHYEKLKGSPEDNEQKQLFVEQHKHYLDEIIEQISEFSKRVILADANLDKVLSAYNKHRD |
| | KPIREQAENIIHLFTLTNLGAPAAFKYFDTTIDRKRYTSTKEVLDATLIHQSITGLYETRIDLSQLGG |
| | DSGGSSGGSKRTADGSEFESPKKKRKVSGGSSGGSVISYDNYVTILDEETLKAWIAKLEKAPVFAFAT |
| | ATDSLDNISANLVGLSFAIEPGVAAYIPVAHDYLDAPDQISRERALELLKPLLEDEKALKVGQNLKYD |
| | RGILANYGIELRGIAFDTMLESYILNSVAGRHDMDSLAERWLKHKTITFEEIAGKGKNQLTFNQIALE |
| | EAGRYAAEDADVTLQLHLKMWPDLQKHKGPLNVFENIEMPLVPVLSRIERNGVKIDPKVLHNHSEELT |
| | LRLAELEKKAHEIAGEEFNLSSTKQLQTILFEKQGIKPLKKTPGGAPSTSEEVLEELALDYPLPKVIL |
| | EYRGLAKLKSTYTDKLPLMINPKTGRVHTSYHQAVTATGRLSSTDPNLQNIPVRNEEGRRIRQAFIAP |
| | EDYVIVSADYSQIELRIMAHLSRDKGLLTAFAEGKDIHRATAAEVFGLPLETVTSEQRRSAKAINFGL |
| | IYGMSAFGLARQLNIPRKEAQKYMDLYFERYPGVLEYMERTRAQAKEQGYVETLDGRRLYLPDIKSSN |
| | GARRAAAERAAINAPMQGTAADIIKRAMIAVDAWLQAEQPRVRMIMQVHDELVFEVHKDDVDAVAKQI |
| | HQLMENCTRLDVPLLVEVGSGENWDQAHSGGSKRTADGSEFESPKKKRKV (SEQ ID NO: 174) |
| pCMV- | MANRTVRRFCFTWNNYPDTAYEKCETFIQKFCKYGIVGEELAPTTGTPHLQGFCNLRKPTRFSTIKKH |
| T7- | LDNSIHIEKANGSDEQNQAYCSKTGIFFEEGSPTKQGQRSGGSSGSETPGTSESATPESSGGSDKKYS |
| PK5222- | IGLDIGTNSVGWAVITDEYKVPSKKFKVLGNTDRHSIKKNLIGALLFDSGETAEATRLKRTARRRYTR |
| XTEN24- | RKNRICYLQEIFSNEMAKVDDSFFHRLEESFLVEEDKKHERHPIFGNIVDEVAYHEKYPTIYHLRKKL |
| nCas9 | VDSTDKADLRLIYLALAHMIKFRGHFLIEGDLNPDNSDVDKLFIQLVQTYNQLFEENPINASGVDAKA |
| (H840A)- | ILSARLSKSRRLENLIAQLPGEKKNGLFGNLIALSLGLTPNFKSNFDLAEDAKLQLSKDTYDDDLDNL |
| SGGSx2 | LAQIGDQYADLFLAAKNLSDAILLSDILRVNTEITKAPLSASMIKRYDEHHQDLTLLKALVRQQLPEK |
| (SEQ ID | YKEIFFDQSKNGYAGYIDGGASQEEFYKFIKPILEKMDGTEELLVKLNREDLLRKQRTFDNGSIPHQI |
| NO: | HLGELHAILRRQEDFYPFLKDNREKIEKILTFRIPYYVGPLARGNSRFAWMTRKSEETITPWNFEEVV |
| 572)- | DKGASAQSFIERMTNFDKNLPNEKVLPKHSLLYEYFTVYNELTKVKYVTEGMRKPAFLSGEQKKAIVD |
| SV40NLS- | LLFKTNRKVTVKQLKEDYFKKIECFDSVEISGVEDRFNASLGTYHDLLKIIKDKDFLDNEENEDILED |
| SGGSx2 | IVLTLTLFEDREMIEERLKTYAHLFDDKVMKQLKRRRYTGWGRLSRKLINGIRDKQSGKTILDFLKSD |
| (SEQ ID | GFANRNFMQLIHDDSLTFKEDIQKAQVSGQGDSLHEHIANLAGSPAIKKGILQTVKVVDELVKVMGRH |
| NO: | KPENIVIEMARENQTTQKGQKNSRERMKRIEEGIKELGSQILKEHPVENTQLQNEKLYLYYLQNGRDM |
| 572)- | YVDQELDINRLSDYDVDAIVPQSFLKDDSIDNKVLTRSDKNRGKSDNVPSEEVVKKMKNYWRQLLNAK |
| Klenow | LITQRKFDNLTKAERGGLSELDKAGFIKRQLVETRQITKHVAQILDSRMNTKYDENDKLIREVKVITL |
| (E.coli)- | KSKLVSDFRKDFQFYKVREINNYHHAHDAYLNAVVGTALIKKYPKLESEFVYGDYKVYDVRKMIAKSE |
| SV40NLS | QEIGKATAKYFFYSNIMNFFKTEITLANGEIRKRPLIETNGETGEIVWDKGRDFATVRKVLSMPQVNI |
| | VKKTEVQTGGFSKESILPKRNSDKLIARKKDWDPKKYGGFDSPTVAYSVLVVAKVEKGKSKKLKSVKE |
| | LLGITIMERSSFEKNPIDFLEAKGYKEVKKDLIIKLPKYSLFELENGRKRMLASAGELQKGNELALPS |
| | KYVNFLYLASHYEKLKGSPEDNEQKQLFVEQHKHYLDEIIEQISEFSKRVILADANLDKVLSAYNKHR |
| | DKPIREQAENIIHLFTLTNLGAPAAFKYFDTTIDRKRYTSTKEVLDATLIHQSITGLYETRIDLSQLG |
| | GDSGGSSGGSKRTADGSEFESPKKKRKVSGGSSGGSVISYDNYVTILDEETLKAWIAKLEKAPVFAFA |
| | TATDSLDNISANLVGLSFAIEPGVAAYIPVAHDYLDAPDQISRERALELLKPLLEDEKALKVGQNLKY |
| | DRGILANYGIELRGIAFDTMLESYILNSVAGRHDMDSLAERWLKHKTITFEEIAGKGKNQLTFNQIAL |
| | EEAGRYAAEDADVTLQLHLKMWPDLQKHKGPLNVFENIEMPLVPVLSRIERNGVKIDPKVLHNHSEEL |
| | TLRLAELEKKAHEIAGEEFNLSSTKQLQTILFEKQGIKPLKKTPGGAPSTSEEVLEELALDYPLPKVI |
| | LEYRGLAKLKSTYTDKLPLMINPKTGRVHTSYHQAVTATGRLSSTDPNLQNIPVRNEEGRRIRQAFIA |
| | PEDYVIVSADYSQIELRIMAHLSRDKGLLTAFAEGKDIHRATAAEVFGLPLETVTSEQRRSAKAINFG |
| | LIYGMSAFGLARQLNIPRKEAQKYMDLYFERYPGVLEYMERTRAQAKEQGYVETLDGRRLYLPDIKSS |
| | NGARRAAAERAAINAPMQGTAADIIKRAMIAVDAWLQAEQPRVRMIMQVHDELVFEVHKDDVDAVAKQ |
| | IHQLMENCTRLDVPLLVEVGSGENWDQAHSGGSKRTADGSEFESPKKKRKV (SEQ ID NO: 175) |
| pCMV- | MSNSTVRRFCFTWNNYTELNYALCQEFIKKYCKYGIVGKELAPTTNTPHLQGFCNLQKPMRFSTIKKR |
| T7- | LDNGIHIEKSMGSDTQNQTYCSKSGEFFEAGDPQCQGKRSGGSSGSETPGTSESATPESSGGSDKKYS |
| PK5510- | IGLDIGTNSVGWAVITDEYKVPSKKFKVLGNTDRHSIKKNLIGALLFDSGETAEATRLKRTARRRYTR |
| XTEN24- | RKNRICYLQEIFSNEMAKVDDSFFHRLEESFLVEEDKKHERHPIFGNIVDEVAYHEKYPTIYHLRKKL |
| nCas9 | VDSTDKADLRLIYLALAHMIKFRGHFLIEGDLNPDNSDVDKLFIQLVQTYNQLFEENPINASGVDAKA |
| (H840A - | ILSARLSKSRRLENLIAQLPGEKKNGLFGNLIALSLGLTPNFKSNFDLAEDAKLQLSKDTYDDDLDNL |
| SGGSx2 | LAQIGDQYADLFLAAKNLSDAILLSDILRVNTEITKAPLSASMIKRYDEHHQDLTLLKALVRQQLPEK |
| (SEQ ID | YKEIFFDQSKNGYAGYIDGGASQEEFYKFIKPILEKMDGTEELLVKLNREDLLRKQRTFDNGSIPHQI |
| NO: | HLGELHAILRRQEDFYPFLKDNREKIEKILTFRIPYYVGPLARGNSRFAWMTRKSEETITPWNFEEVV |
| 572)- | DKGASAQSFIERMTNFDKNLPNEKVLPKHSLLYEYFTVYNELTKVKYVTEGMRKPAFLSGEQKKAIVD |
| SV40NLS- | LLFKTNRKVTVKQLKEDYFKKIECFDSVEISGVEDRFNASLGTYHDLLKIIKDKDFLDNEENEDILED |
| SGGSx2 | IVLTLTLFEDREMIEERLKTYAHLFDDKVMKQLKRRRYTGWGRLSRKLINGIRDKQSGKTILDFLKSD |
| (SEQ ID | GFANRNFMQLIHDDSLTFKEDIQKAQVSGQGDSLHEHIANLAGSPAIKKGILQTVKVVDELVKVMGRH |
| NO: | KPENIVIEMARENQTTQKGQKNSRERMKRIEEGIKELGSQILKEHPVENTQLQNEKLYLYYLQNGRDM |
| 572)- | YVDQELDINRLSDYDVDAIVPQSFLKDDSIDNKVLTRSDKNRGKSDNVPSEEVVKKMKNYWRQLLNAK |
| Klenow | LITQRKFDNLTKAERGGLSELDKAGFIKRQLVETRQITKHVAQILDSRMNTKYDENDKLIREVKVITL |
| (E.coli)- | KSKLVSDFRKDFQFYKVREINNYHHAHDAYLNAVVGTALIKKYPKLESEFVYGDYKVYDVRKMIAKSE |
| SV40NLS | QEIGKATAKYFFYSNIMNFFKTEITLANGEIRKRPLIETNGETGEIVWDKGRDFATVRKVLSMPQVNI |
| | VKKTEVQTGGFSKESILPKRNSDKLIARKKDWDPKKYGGFDSPTVAYSVLVVAKVEKGKSKKLKSVKE |
| | LLGITIMERSSFEKNPIDFLEAKGYKEVKKDLIIKLPKYSLFELENGRKRMLASAGELQKGNELALPS |
| | KYVNFLYLASHYEKLKGSPEDNEQKQLFVEQHKHYLDEIIEQISEFSKRVILADANLDKVLSAYNKHR |
| | DKPIREQAENIIHLFTLTNLGAPAAFKYFDTTIDRKRYTSTKEVLDATLIHQSITGLYETRIDLSQLG |
| | GDSGGSSGGSKRTADGSEFESPKKKRKVSGGSSGGSVISYDNYVTILDEETLKAWIAKLEKAPVFAFA |
| | TATDSLDNISANLVGLSFAIEPGVAAYIPVAHDYLDAPDQISRERALELLKPLLEDEKALKVGQNLKY |
| | DRGILANYGIELRGIAFDTMLESYILNSVAGRHDMDSLAERWLKHKTITFEEIAGKGKNQLTFNQIAL |
| | EEAGRYAAEDADVTLQLHLKMWPDLQKHKGPLNVFENIEMPLVPVLSRIERNGVKIDPKVLHNHSEEL |
| | TLRLAELEKKAHEIAGEEFNLSSTKQLQTILFEKQGIKPLKKTPGGAPSTSEEVLEELALDYPLPKVI |
| | LEYRGLAKLKSTYTDKLPLMINPKTGRVHTSYHQAVTATGRLSSTDPNLQNIPVRNEEGRRIRQAFIA |
| | PEDYVIVSADYSQIELRIMAHLSRDKGLLTAFAEGKDIHRATAAEVFGLPLETVTSEQRRSAKAINFG |
| | LIYGMSAFGLARQLNIPRKEAQKYMDLYFERYPGVLEYMERTRAQAKEQGYVETLDGRRLYLPDIKSS |
| | NGARRAAAERAAINAPMQGTAADIIKRAMIAVDAWLQAEQPRVRMIMQVHDELVFEVHKDDVDAVAKQ |
| | IHQLMENCTRLDVPLLVEVGSGENWDQAHSGGSKRTADGSEFESPKKKRKV (SEQ ID NO: 176) |
| pCMV- | MAVSSRKQTNSTLRRFCWTLNNYTEEDVTTLQKDLAELCKFAIFGRETCPNTGTKHLQGFCNLQRPKR |
| T7-T25- | FSSIRKLFKERAHIEKAKGSDFDNKAYCSKSGEVWMHGEPSSQGARSGGSSGSETPGTSESATPESSG |
| XTEN24- | GSDKKYSIGLDIGTNSVGWAVITDEYKVPSKKFKVLGNTDRHSIKKNLIGALLFDSGETAEATRLKRT |
| nCas9 | ARRRYTRRKNRICYLQEIFSNEMAKVDDSFFHRLEESFLVEEDKKHERHPIFGNIVDEVAYHEKYPTI |
| (H840A)- | YHLRKKLVDSTDKADLRLIYLALAHMIKFRGHFLIEGDLNPDNSDVDKLFIQLVQTYNQLFEENPINA |
| SGGSx2 | SGVDAKAILSARLSKSRRLENLIAQLPGEKKNGLFGNLIALSLGLTPNFKSNFDLAEDAKLQLSKDTY |
| (SEQ ID | DDDLDNLLAQIGDQYADLFLAAKNLSDAILLSDILRVNTEITKAPLSASMIKRYDEHHQDLTLLKALV |
| NO: | RQQLPEKYKEIFFDQSKNGYAGYIDGGASQEEFYKFIKPILEKMDGTEELLVKLNREDLLRKQRTFDN |
| 572)- | GSIPHQIHLGELHAILRRQEDFYPFLKDNREKIEKILTFRIPYYVGPLARGNSRFAWMTRKSEETITP |
| SV40NLS- | WNFEEVVDKGASAQSFIERMTNFDKNLPNEKVLPKHSLLYEYFTVYNELTKVKYVTEGMRKPAFLSGE |
| SGGSx2 | QKKAIVDLLFKTNRKVTVKQLKEDYFKKIECFDSVEISGVEDRFNASLGTYHDLLKIIKDKDFLDNEE |
| (SEQ ID | NEDILEDIVLTLTLFEDREMIEERLKTYAHLFDDKVMKQLKRRRYTGWGRLSRKLINGIRDKQSGKTI |
| NO: | LDFLKSDGFANRNFMQLIHDDSLTFKEDIQKAQVSGQGDSLHEHIANLAGSPAIKKGILQTVKVVDEL |
| 572)- | VKVMGRHKPENIVIEMARENQTTQKGQKNSRERMKRIEEGIKELGSQILKEHPVENTQLQNEKLYLYY |
| Klenow | LQNGRDMYVDQELDINRLSDYDVDAIVPQSFLKDDSIDNKVLTRSDKNRGKSDNVPSEEVVKKMKNYW |
| (E.coli)- | RQLLNAKLITQRKFDNLTKAERGGLSELDKAGFIKRQLVETRQITKHVAQILDSRMNTKYDENDKLIR |
| SV40NLS | EVKVITLKSKLVSDFRKDFQFYKVREINNYHHAHDAYLNAVVGTALIKKYPKLESEFVYGDYKVYDVR |
| | KMIAKSEQEIGKATAKYFFYSNIMNFFKTEITLANGEIRKRPLIETNGETGEIVWDKGRDFATVRKVL |
| | SMPQVNIVKKTEVQTGGFSKESILPKRNSDKLIARKKDWDPKKYGGFDSPTVAYSVLVVAKVEKGKSK |
| | KLKSVKELLGITIMERSSFEKNPIDFLEAKGYKEVKKDLIIKLPKYSLFELENGRKRMLASAGELQKG |
| | NELALPSKYVNFLYLASHYEKLKGSPEDNEQKQLFVEQHKHYLDEIIEQISEFSKRVILADANLDKVL |
| | SAYNKHRDKPIREQAENIIHLFTLTNLGAPAAFKYFDTTIDRKRYTSTKEVLDATLIHQSITGLYETR |
| | IDLSQLGGDSGGSSGGSKRTADGSEFESPKKKRKVSGGSSGGSVISYDNYVTILDEETLKAWIAKLEK |
| | APVFAFATATDSLDNISANLVGLSFAIEPGVAAYIPVAHDYLDAPDQISRERALELLKPLLEDEKALK |
| | VGQNLKYDRGILANYGIELRGIAFDTMLESYILNSVAGRHDMDSLAERWLKHKTITFEEIAGKGKNQL |
| | TFNQIALEEAGRYAAEDADVTLQLHLKMWPDLQKHKGPLNVFENIEMPLVPVLSRIERNGVKIDPKVL |
| | HNHSEELTLRLAELEKKAHEIAGEEFNLSSTKQLQTILFEKQGIKPLKKTPGGAPSTSEEVLEELALD |
| | YPLPKVILEYRGLAKLKSTYTDKLPLMINPKTGRVHTSYHQAVTATGRLSSTDPNLQNIPVRNEEGRR |
| | IRQAFIAPEDYVIVSADYSQIELRIMAHLSRDKGLLTAFAEGKDIHRATAAEVFGLPLETVTSEQRRS |
| | AKAINFGLIYGMSAFGLARQLNIPRKEAQKYMDLYFERYPGVLEYMERTRAQAKEQGYVETLDGRRLY |
| | LPDIKSSNGARRAAAERAAINAPMQGTAADIIKRAMIAVDAWLQAEQPRVRMIMQVHDELVFEVHKDD |
| | VDAVAKQIHQLMENCTRLDVPLLVEVGSGENWDQAHSGGSKRTADGSEFESPKKKRKV (SEQ ID |
| | NO: 177) |
| pCMV- | MSNRTVRKFCFTWNNYEFDAYAKCETFLNNFAKYGIVGEELCPSTGTPHLQGYVNLIKPTRFSTIKKH |
| T7- | LHNAIHIEKANGSDEQNQTYCRKSGIFFEKGEPIKQGQRSGGSSGSETPGTSESATPESSGGSDKKYS |
| Chimp11- | IGLDIGTNSVGWAVITDEYKVPSKKFKVLGNTDRHSIKKNLIGALLFDSGETAEATRLKRTARRRYTR |
| XTEN24- | RKNRICYLQEIFSNEMAKVDDSFFHRLEESFLVEEDKKHERHPIFGNIVDEVAYHEKYPTIYHLRKKL |
| nCas9 | VDSTDKADLRLIYLALAHMIKFRGHFLIEGDLNPDNSDVDKLFIQLVQTYNQLFEENPINASGVDAKA |
| (H840A)- | ILSARLSKSRRLENLIAQLPGEKKNGLFGNLIALSLGLTPNFKSNFDLAEDAKLQLSKDTYDDDLDNL |
| SGGSx2 | LAQIGDQYADLFLAAKNLSDAILLSDILRVNTEITKAPLSASMIKRYDEHHQDLTLLKALVRQQLPEK |
| (SEQ ID | YKEIFFDQSKNGYAGYIDGGASQEEFYKFIKPILEKMDGTEELLVKLNREDLLRKQRTFDNGSIPHQI |
| NO: | HLGELHAILRRQEDFYPFLKDNREKIEKILTFRIPYYVGPLARGNSRFAWMTRKSEETITPWNFEEVV |
| 572)- | DKGASAQSFIERMTNFDKNLPNEKVLPKHSLLYEYFTVYNELTKVKYVTEGMRKPAFLSGEQKKAIVD |
| SV40NLS- | LLFKTNRKVTVKQLKEDYFKKIECFDSVEISGVEDRFNASLGTYHDLLKIIKDKDFLDNEENEDILED |
| SGGSx2 | IVLTLTLFEDREMIEERLKTYAHLFDDKVMKQLKRRRYTGWGRLSRKLINGIRDKQSGKTILDFLKSD |
| (SEQ ID | GFANRNFMQLIHDDSLTFKEDIQKAQVSGQGDSLHEHIANLAGSPAIKKGILQTVKVVDELVKVMGRH |
| NO: | KPENIVIEMARENQTTQKGQKNSRERMKRIEEGIKELGSQILKEHPVENTQLQNEKLYLYYLQNGRDM |
| 572)- | YVDQELDINRLSDYDVDAIVPQSFLKDDSIDNKVLTRSDKNRGKSDNVPSEEVVKKMKNYWRQLLNAK |
| Klenow | LITQRKFDNLTKAERGGLSELDKAGFIKRQLVETRQITKHVAQILDSRMNTKYDENDKLIREVKVITL |
| (E.coli)- | KSKLVSDFRKDFQFYKVREINNYHHAHDAYLNAVVGTALIKKYPKLESEFVYGDYKVYDVRKMIAKSE |
| SV40NLS | QEIGKATAKYFFYSNIMNFFKTEITLANGEIRKRPLIETNGETGEIVWDKGRDFATVRKVLSMPQVNI |
| | VKKTEVQTGGFSKESILPKRNSDKLIARKKDWDPKKYGGFDSPTVAYSVLVVAKVEKGKSKKLKSVKE |
| | LLGITIMERSSFEKNPIDFLEAKGYKEVKKDLIIKLPKYSLFELENGRKRMLASAGELQKGNELALPS |
| | KYVNFLYLASHYEKLKGSPEDNEQKQLFVEQHKHYLDEIIEQISEFSKRVILADANLDKVLSAYNKHR |
| | DKPIREQAENIIHLFTLTNLGAPAAFKYFDTTIDRKRYTSTKEVLDATLIHQSITGLYETRIDLSQLG |
| | GDSGGSSGGSKRTADGSEFESPKKKRKVSGGSSGGSVISYDNYVTILDEETLKAWIAKLEKAPVFAFA |
| | TATDSLDNISANLVGLSFAIEPGVAAYIPVAHDYLDAPDQISRERALELLKPLLEDEKALKVGQNLKY |
| | DRGILANYGIELRGIAFDTMLESYILNSVAGRHDMDSLAERWLKHKTITFEEIAGKGKNQLTFNQIAL |
| | EEAGRYAAEDADVTLQLHLKMWPDLQKHKGPLNVFENIEMPLVPVLSRIERNGVKIDPKVLHNHSEEL |
| | TLRLAELEKKAHEIAGEEFNLSSTKQLQTILFEKQGIKPLKKTPGGAPSTSEEVLEELALDYPLPKVI |
| | LEYRGLAKLKSTYTDKLPLMINPKTGRVHTSYHQAVTATGRLSSTDPNLQNIPVRNEEGRRIRQAFIA |
| | PEDYVIVSADYSQIELRIMAHLSRDKGLLTAFAEGKDIHRATAAEVFGLPLETVTSEQRRSAKAINFG |
| | LIYGMSAFGLARQLNIPRKEAQKYMDLYFERYPGVLEYMERTRAQAKEQGYVETLDGRRLYLPDIKSS |
| | NGARRAAAERAAINAPMQGTAADIIKRAMIAVDAWLQAEQPRVRMIMQVHDELVFEVHKDDVDAVAKQ |
| | IHQLMENCTRLDVPLLVEVGSGENWDQAHSGGSKRTADGSEFESPKKKRKV (SEQ ID NO: 178) |
| pCMV- | MAVRGSAAKRWCFTLNNPTEEEIAAVKAWQHSEYHYAIVGKEKGEQGTPHLQGFIHLKKKVRLTSLKK |
| T7- | VLQRAHWEKARGSDEDNEKYCSKEGDVILTIGIPVKGNRSGGSSGSETPGTSESATPESSGGSDKKYS |
| StCV- | IGLDIGTNSVGWAVITDEYKVPSKKFKVLGNTDRHSIKKNLIGALLFDSGETAEATRLKRTARRRYTR |
| XTEN24- | RKNRICYLQEIFSNEMAKVDDSFFHRLEESFLVEEDKKHERHPIFGNIVDEVAYHEKYPTIYHLRKKL |
| nCas9 | VDSTDKADLRLIYLALAHMIKFRGHFLIEGDLNPDNSDVDKLFIQLVQTYNQLFEENPINASGVDAKA |
| (H840A)- | ILSARLSKSRRLENLIAQLPGEKKNGLFGNLIALSLGLTPNFKSNFDLAEDAKLQLSKDTYDDDLDNL |
| SGGSx2 | LAQIGDQYADLFLAAKNLSDAILLSDILRVNTEITKAPLSASMIKRYDEHHQDLTLLKALVRQQLPEK |
| (SEQ ID | YKEIFFDQSKNGYAGYIDGGASQEEFYKFIKPILEKMDGTEELLVKLNREDLLRKQRTFDNGSIPHQI |
| NO: | HLGELHAILRRQEDFYPFLKDNREKIEKILTFRIPYYVGPLARGNSRFAWMTRKSEETITPWNFEEVV |
| 572)- | DKGASAQSFIERMTNFDKNLPNEKVLPKHSLLYEYFTVYNELTKVKYVTEGMRKPAFLSGEQKKAIVD |
| SV40NLS- | LLFKTNRKVTVKQLKEDYFKKIECFDSVEISGVEDRFNASLGTYHDLLKIIKDKDFLDNEENEDILED |
| SGGSX2 | IVLTLTLFEDREMIEERLKTYAHLFDDKVMKQLKRRRYTGWGRLSRKLINGIRDKQSGKTILDFLKSD |
| (SEQ ID | GFANRNFMQLIHDDSLTFKEDIQKAQVSGQGDSLHEHIANLAGSPAIKKGILQTVKVVDELVKVMGRH |
| NO: | KPENIVIEMARENQTTQKGQKNSRERMKRIEEGIKELGSQILKEHPVENTQLQNEKLYLYYLQNGRDM |
| 572)- | YVDQELDINRLSDYDVDAIVPQSFLKDDSIDNKVLTRSDKNRGKSDNVPSEEVVKKMKNYWRQLLNAK |
| Klenow | LITQRKFDNLTKAERGGLSELDKAGFIKRQLVETRQITKHVAQILDSRMNTKYDENDKLIREVKVITL |
| (E.coli)- | KSKLVSDFRKDFQFYKVREINNYHHAHDAYLNAVVGTALIKKYPKLESEFVYGDYKVYDVRKMIAKSE |
| SV40NLS | QEIGKATAKYFFYSNIMNFFKTEITLANGEIRKRPLIETNGETGEIVWDKGRDFATVRKVLSMPQVNI |
| | VKKTEVQTGGFSKESILPKRNSDKLIARKKDWDPKKYGGFDSPTVAYSVLVVAKVEKGKSKKLKSVKE |
| | LLGITIMERSSFEKNPIDFLEAKGYKEVKKDLIIKLPKYSLFELENGRKRMLASAGELQKGNELALPS |
| | KYVNFLYLASHYEKLKGSPEDNEQKQLFVEQHKHYLDEIIEQISEFSKRVILADANLDKVLSAYNKHR |
| | DKPIREQAENIIHLFTLTNLGAPAAFKYFDTTIDRKRYTSTKEVLDATLIHQSITGLYETRIDLSQLG |
| | GDSGGSSGGSKRTADGSEFESPKKKRKVSGGSSGGSVISYDNYVTILDEETLKAWIAKLEKAPVFAFA |
| | TATDSLDNISANLVGLSFAIEPGVAAYIPVAHDYLDAPDQISRERALELLKPLLEDEKALKVGQNLKY |
| | DRGILANYGIELRGIAFDTMLESYILNSVAGRHDMDSLAERWLKHKTITFEEIAGKGKNQLTFNQIAL |
| | EEAGRYAAEDADVTLQLHLKMWPDLQKHKGPLNVFENIEMPLVPVLSRIERNGVKIDPKVLHNHSEEL |
| | TLRLAELEKKAHEIAGEEFNLSSTKQLQTILFEKQGIKPLKKTPGGAPSTSEEVLEELALDYPLPKVI |
| | LEYRGLAKLKSTYTDKLPLMINPKTGRVHTSYHQAVTATGRLSSTDPNLQNIPVRNEEGRRIRQAFIA |
| | PEDYVIVSADYSQIELRIMAHLSRDKGLLTAFAEGKDIHRATAAEVFGLPLETVTSEQRRSAKAINFG |
| | LIYGMSAFGLARQLNIPRKEAQKYMDLYFERYPGVLEYMERTRAQAKEQGYVETLDGRRLYLPDIKSS |
| | NGARRAAAERAAINAPMQGTAADIIKRAMIAVDAWLQAEQPRVRMIMQVHDELVFEVHKDDVDAVAKQ |
| | IHQLMENCTRLDVPLLVEVGSGENWDQAHSGGSKRTADGSEFESPKKKRKV (SEQ ID NO: 179) |
| pCMV- | MAPVRAAAAKRWCFTLNNYTAEEEAKVRALLPGEFHFAICGKERGEQGTPHLQGFLHFKKKQRLSALK |
| T7- | KLLARAHWEKARGSDHDNEEYCSKENDVILTIGEPVQGNRSGGSSGSETPGTSESATPESSGGSDKKY |
| CaCV- | SIGLDIGTNSVGWAVITDEYKVPSKKFKVLGNTDRHSIKKNLIGALLFDSGETAEATRLKRTARRRYT |
| XTEN24- | RRKNRICYLQEIFSNEMAKVDDSFFHRLEESFLVEEDKKHERHPIFGNIVDEVAYHEKYPTIYHLRKK |
| nCas9 | LVDSTDKADLRLIYLALAHMIKFRGHFLIEGDLNPDNSDVDKLFIQLVQTYNQLFEENPINASGVDAK |
| (H840A)- | AILSARLSKSRRLENLIAQLPGEKKNGLFGNLIALSLGLTPNFKSNFDLAEDAKLQLSKDTYDDDLDN |
| SGGSX2 | LLAQIGDQYADLFLAAKNLSDAILLSDILRVNTEITKAPLSASMIKRYDEHHQDLTLLKALVRQQLPE |
| (SEQ ID | KYKEIFFDQSKNGYAGYIDGGASQEEFYKFIKPILEKMDGTEELLVKLNREDLLRKQRTFDNGSIPHQ |
| NO: | IHLGELHAILRRQEDFYPFLKDNREKIEKILTFRIPYYVGPLARGNSRFAWMTRKSEETITPWNFEEV |
| 572)- | VDKGASAQSFIERMTNFDKNLPNEKVLPKHSLLYEYFTVYNELTKVKYVTEGMRKPAFLSGEQKKAIV |
| SV40NLS- | DLLFKTNRKVTVKQLKEDYFKKIECFDSVEISGVEDRFNASLGTYHDLLKIIKDKDFLDNEENEDILE |
| SGGSx2 | DIVLTLTLFEDREMIEERLKTYAHLFDDKVMKQLKRRRYTGWGRLSRKLINGIRDKQSGKTILDFLKS |
| (SEQ ID | DGFANRNFMQLIHDDSLTFKEDIQKAQVSGQGDSLHEHIANLAGSPAIKKGILQTVKVVDELVKVMGR |
| NO: | HKPENIVIEMARENQTTQKGQKNSRERMKRIEEGIKELGSQILKEHPVENTQLQNEKLYLYYLQNGRD |
| 572)- | MYVDQELDINRLSDYDVDAIVPQSFLKDDSIDNKVLTRSDKNRGKSDNVPSEEVVKKMKNYWRQLLNA |
| Klenow | KLITQRKFDNLTKAERGGLSELDKAGFIKRQLVETRQITKHVAQILDSRMNTKYDENDKLIREVKVIT |
| (E.coli)- | LKSKLVSDFRKDFQFYKVREINNYHHAHDAYLNAVVGTALIKKYPKLESEFVYGDYKVYDVRKMIAKS |
| SV40NLS | EQEIGKATAKYFFYSNIMNFFKTEITLANGEIRKRPLIETNGETGEIVWDKGRDFATVRKVLSMPQVN |
| | IVKKTEVQTGGFSKESILPKRNSDKLIARKKDWDPKKYGGFDSPTVAYSVLVVAKVEKGKSKKLKSVK |
| | ELLGITIMERSSFEKNPIDFLEAKGYKEVKKDLIIKLPKYSLFELENGRKRMLASAGELQKGNELALP |
| | SKYVNFLYLASHYEKLKGSPEDNEQKQLFVEQHKHYLDEIIEQISEFSKRVILADANLDKVLSAYNKH |
| | RDKPIREQAENIIHLFTLTNLGAPAAFKYFDTTIDRKRYTSTKEVLDATLIHQSITGLYETRIDLSQL |
| | GGDSGGSSGGSKRTADGSEFESPKKKRKVSGGSSGGSVISYDNYVTILDEETLKAWIAKLEKAPVFAF |
| | ATATDSLDNISANLVGLSFAIEPGVAAYIPVAHDYLDAPDQISRERALELLKPLLEDEKALKVGQNLK |
| | YDRGILANYGIELRGIAFDTMLESYILNSVAGRHDMDSLAERWLKHKTITFEEIAGKGKNQLTFNQIA |
| | LEEAGRYAAEDADVTLQLHLKMWPDLQKHKGPLNVFENIEMPLVPVLSRIERNGVKIDPKVLHNHSEE |
| | LTLRLAELEKKAHEIAGEEFNLSSTKQLQTILFEKQGIKPLKKTPGGAPSTSEEVLEELALDYPLPKV |
| | ILEYRGLAKLKSTYTDKLPLMINPKTGRVHTSYHQAVTATGRLSSTDPNLQNIPVRNEEGRRIRQAFI |
| | APEDYVIVSADYSQIELRIMAHLSRDKGLLTAFAEGKDIHRATAAEVFGLPLETVTSEQRRSAKAINF |
| | GLIYGMSAFGLARQLNIPRKEAQKYMDLYFERYPGVLEYMERTRAQAKEQGYVETLDGRRLYLPDIKS |
| | SNGARRAAAERAAINAPMQGTAADIIKRAMIAVDAWLQAEQPRVRMIMQVHDELVFEVHKDDVDAVAK |
| | QIHQLMENCTRLDVPLLVEVGSGENWDQAHSGGSKRTADGSEFESPKKKRKV (SEQ ID NO: 180) |
| pCMV- | MAPIKRPAPCKRWCFTLNNFTDDEVQKVVSLQPDEVHYAIVGRETGAQGTPHLQGYLHLKKKKRLTSM |
| T7- | KEFLPRAHWEVARGSDEDNEAYCSKEGDVILTLGIPAKGNRSGGSSGSETPGTSESATPESSGGSDKK |
| Chimp17- | YSIGLDIGTNSVGWAVITDEYKVPSKKFKVLGNTDRHSIKKNLIGALLFDSGETAEATRLKRTARRRY |
| XTEN24- | TRRKNRICYLQEIFSNEMAKVDDSFFHRLEESFLVEEDKKHERHPIFGNIVDEVAYHEKYPTIYHLRK |
| nCas9 | KLVDSTDKADLRLIYLALAHMIKFRGHFLIEGDLNPDNSDVDKLFIQLVQTYNQLFEENPINASGVDA |
| (H840A)- | KAILSARLSKSRRLENLIAQLPGEKKNGLFGNLIALSLGLTPNFKSNFDLAEDAKLQLSKDTYDDDLD |
| SGGSx2 | NLLAQIGDQYADLFLAAKNLSDAILLSDILRVNTEITKAPLSASMIKRYDEHHQDLTLLKALVRQQLP |
| (SEQ ID | EKYKEIFFDQSKNGYAGYIDGGASQEEFYKFIKPILEKMDGTEELLVKLNREDLLRKQRTFDNGSIPH |
| NO: | QIHLGELHAILRRQEDFYPFLKDNREKIEKILTFRIPYYVGPLARGNSRFAWMTRKSEETITPWNFEE |
| 572)- | VVDKGASAQSFIERMTNFDKNLPNEKVLPKHSLLYEYFTVYNELTKVKYVTEGMRKPAFLSGEQKKAI |
| SV40NLS- | VDLLFKTNRKVTVKQLKEDYFKKIECFDSVEISGVEDRFNASLGTYHDLLKIIKDKDFLDNEENEDIL |
| SGGSx2 | EDIVLTLTLFEDREMIEERLKTYAHLFDDKVMKQLKRRRYTGWGRLSRKLINGIRDKQSGKTILDFLK |
| (SEQ ID | SDGFANRNFMQLIHDDSLTFKEDIQKAQVSGQGDSLHEHIANLAGSPAIKKGILQTVKVVDELVKVMG |
| NO: | RHKPENIVIEMARENQTTQKGQKNSRERMKRIEEGIKELGSQILKEHPVENTQLQNEKLYLYYLQNGR |
| 572)- | DMYVDQELDINRLSDYDVDAIVPQSFLKDDSIDNKVLTRSDKNRGKSDNVPSEEVVKKMKNYWRQLLN |
| Klenow | AKLITQRKFDNLTKAERGGLSELDKAGFIKRQLVETRQITKHVAQILDSRMNTKYDENDKLIREVKVI |
| (E.coli)- | TLKSKLVSDFRKDFQFYKVREINNYHHAHDAYLNAVVGTALIKKYPKLESEFVYGDYKVYDVRKMIAK |
| SV40NLS | SEQEIGKATAKYFFYSNIMNFFKTEITLANGEIRKRPLIETNGETGEIVWDKGRDFATVRKVLSMPQV |
| | NIVKKTEVQTGGFSKESILPKRNSDKLIARKKDWDPKKYGGFDSPTVAYSVLVVAKVEKGKSKKLKSV |
| | KELLGITIMERSSFEKNPIDFLEAKGYKEVKKDLIIKLPKYSLFELENGRKRMLASAGELQKGNELAL |
| | PSKYVNFLYLASHYEKLKGSPEDNEQKQLFVEQHKHYLDEIIEQISEFSKRVILADANLDKVLSAYNK |
| | HRDKPIREQAENIIHLFTLTNLGAPAAFKYFDTTIDRKRYTSTKEVLDATLIHQSITGLYETRIDLSQ |
| | LGGDSGGSSGGSKRTADGSEFESPKKKRKVSGGSSGGSVISYDNYVTILDEETLKAWIAKLEKAPVFA |
| | FATATDSLDNISANLVGLSFAIEPGVAAYIPVAHDYLDAPDQISRERALELLKPLLEDEKALKVGQNL |
| | KYDRGILANYGIELRGIAFDTMLESYILNSVAGRHDMDSLAERWLKHKTITFEEIAGKGKNQLTFNQI |
| | ALEEAGRYAAEDADVTLQLHLKMWPDLQKHKGPLNVFENIEMPLVPVLSRIERNGVKIDPKVLHNHSE |
| | ELTLRLAELEKKAHEIAGEEFNLSSTKQLQTILFEKQGIKPLKKTPGGAPSTSEEVLEELALDYPLPK |
| | VILEYRGLAKLKSTYTDKLPLMINPKTGRVHTSYHQAVTATGRLSSTDPNLQNIPVRNEEGRRIRQAF |
| | IAPEDYVIVSADYSQIELRIMAHLSRDKGLLTAFAEGKDIHRATAAEVFGLPLETVTSEQRRSAKAIN |
| | FGLIYGMSAFGLARQLNIPRKEAQKYMDLYFERYPGVLEYMERTRAQAKEQGYVETLDGRRLYLPDIK |
| | SSNGARRAAAERAAINAPMQGTAADIIKRAMIAVDAWLQAEQPRVRMIMQVHDELVFEVHKDDVDAVA |
| | KQIHQLMENCTRLDVPLLVEVGSGENWDQAHSGGSKRTADGSEFESPKKKRKV (SEQ ID |
| | NO: 181) |
| pCMV- | MAKNGNYSYKRWVFTINNPTFEDYCAVVEFCNLDNCKFAIVGEEKGEKEGTPHLQGFLSLRKNAKAAA |
| T7- | LEENLGGRAWLSRARGSDEDNEEYCSKESTYLRVGEPNRKGRSSGGSSGSETPGTSESATPESSGGSD |
| GoCV- | KKYSIGLDIGTNSVGWAVITDEYKVPSKKFKVLGNTDRHSIKKNLIGALLFDSGETAEATRLKRTARR |
| XTEN24- | RYTRRKNRICYLQEIFSNEMAKVDDSFFHRLEESFLVEEDKKHERHPIFGNIVDEVAYHEKYPTIYHL |
| nCas9 | RKKLVDSTDKADLRLIYLALAHMIKFRGHFLIEGDLNPDNSDVDKLFIQLVQTYNQLFEENPINASGV |
| (H840A)- | DAKAILSARLSKSRRLENLIAQLPGEKKNGLFGNLIALSLGLTPNFKSNFDLAEDAKLQLSKDTYDDD |
| SGGSx2 | LDNLLAQIGDQYADLFLAAKNLSDAILLSDILRVNTEITKAPLSASMIKRYDEHHQDLTLLKALVRQQ |
| (SEQ ID | LPEKYKEIFFDQSKNGYAGYIDGGASQEEFYKFIKPILEKMDGTEELLVKLNREDLLRKQRTFDNGSI |
| NO: | PHQIHLGELHAILRRQEDFYPFLKDNREKIEKILTFRIPYYVGPLARGNSRFAWMTRKSEETITPWNF |
| 572)- | EEVVDKGASAQSFIERMTNFDKNLPNEKVLPKHSLLYEYFTVYNELTKVKYVTEGMRKPAFLSGEQKK |
| SV40NLS- | AIVDLLFKTNRKVTVKQLKEDYFKKIECFDSVEISGVEDRFNASLGTYHDLLKIIKDKDFLDNEENED |
| SGGSx2 | ILEDIVLTLTLFEDREMIEERLKTYAHLFDDKVMKQLKRRRYTGWGRLSRKLINGIRDKQSGKTILDF |
| (SEQ ID | LKSDGFANRNFMQLIHDDSLTFKEDIQKAQVSGQGDSLHEHIANLAGSPAIKKGILQTVKVVDELVKV |
| NO: | MGRHKPENIVIEMARENQTTQKGQKNSRERMKRIEEGIKELGSQILKEHPVENTQLQNEKLYLYYLQN |
| 572)- | GRDMYVDQELDINRLSDYDVDAIVPQSFLKDDSIDNKVLTRSDKNRGKSDNVPSEEVVKKMKNYWRQL |
| Klenow | LNAKLITQRKFDNLTKAERGGLSELDKAGFIKRQLVETRQITKHVAQILDSRMNTKYDENDKLIREVK |
| (E.coli)- | VITLKSKLVSDFRKDFQFYKVREINNYHHAHDAYLNAVVGTALIKKYPKLESEFVYGDYKVYDVRKMI |
| SV40NLS | AKSEQEIGKATAKYFFYSNIMNFFKTEITLANGEIRKRPLIETNGETGEIVWDKGRDFATVRKVLSMP |
| | QVNIVKKTEVQTGGFSKESILPKRNSDKLIARKKDWDPKKYGGFDSPTVAYSVLVVAKVEKGKSKKLK |
| | SVKELLGITIMERSSFEKNPIDFLEAKGYKEVKKDLIIKLPKYSLFELENGRKRMLASAGELQKGNEL |
| | ALPSKYVNFLYLASHYEKLKGSPEDNEQKQLFVEQHKHYLDEIIEQISEFSKRVILADANLDKVLSAY |
| | NKHRDKPIREQAENIIHLFTLTNLGAPAAFKYFDTTIDRKRYTSTKEVLDATLIHQSITGLYETRIDL |
| | SQLGGDSGGSSGGSKRTADGSEFESPKKKRKVSGGSSGGSVISYDNYVTILDEETLKAWIAKLEKAPV |
| | FAFATATDSLDNISANLVGLSFAIEPGVAAYIPVAHDYLDAPDQISRERALELLKPLLEDEKALKVGQ |
| | NLKYDRGILANYGIELRGIAFDTMLESYILNSVAGRHDMDSLAERWLKHKTITFEEIAGKGKNQLTFN |
| | QIALEEAGRYAAEDADVTLQLHLKMWPDLQKHKGPLNVFENIEMPLVPVLSRIERNGVKIDPKVLHNH |
| | SEELTLRLAELEKKAHEIAGEEFNLSSTKQLQTILFEKQGIKPLKKTPGGAPSTSEEVLEELALDYPL |
| | PKVILEYRGLAKLKSTYTDKLPLMINPKTGRVHTSYHQAVTATGRLSSTDPNLQNIPVRNEEGRRIRQ |
| | AFIAPEDYVIVSADYSQIELRIMAHLSRDKGLLTAFAEGKDIHRATAAEVFGLPLETVTSEQRRSAKA |
| | INFGLIYGMSAFGLARQLNIPRKEAQKYMDLYFERYPGVLEYMERTRAQAKEQGYVETLDGRRLYLPD |
| | IKSSNGARRAAAERAAINAPMQGTAADIIKRAMIAVDAWLQAEQPRVRMIMQVHDELVFEVHKDDVDA |
| | VAKQIHQLMENCTRLDVPLLVEVGSGENWDQAHSGGSKRTADGSEFESPKKKRKV (SEQ ID |
| | NO: 182) |
| pCMV- | MAKKSDYGYKRWVFTINNPTFEDYVSVIEFCTAENCKFAIVGEEKGEKEGTPHLQGFLSLRKNARAAA |
| T7- | LEENLGGRAWLSRAVGSDEENEEYCSKETTYLRVGTPNRKGRSSGGSSGSETPGTSESATPESSGGSD |
| SwCV- | KKYSIGLDIGTNSVGWAVITDEYKVPSKKFKVLGNTDRHSIKKNLIGALLFDSGETAEATRLKRTARR |
| XTEN24- | RYTRRKNRICYLQEIFSNEMAKVDDSFFHRLEESFLVEEDKKHERHPIFGNIVDEVAYHEKYPTIYHL |
| nCas9 | RKKLVDSTDKADLRLIYLALAHMIKFRGHFLIEGDLNPDNSDVDKLFIQLVQTYNQLFEENPINASGV |
| (H840A)- | DAKAILSARLSKSRRLENLIAQLPGEKKNGLFGNLIALSLGLTPNFKSNFDLAEDAKLQLSKDTYDDD |
| SGGSx2 | LDNLLAQIGDQYADLFLAAKNLSDAILLSDILRVNTEITKAPLSASMIKRYDEHHQDLTLLKALVRQQ |
| (SEQ ID | LPEKYKEIFFDQSKNGYAGYIDGGASQEEFYKFIKPILEKMDGTEELLVKLNREDLLRKQRTFDNGSI |
| NO: | PHQIHLGELHAILRRQEDFYPFLKDNREKIEKILTFRIPYYVGPLARGNSRFAWMTRKSEETITPWNF |
| 572)- | EEVVDKGASAQSFIERMTNFDKNLPNEKVLPKHSLLYEYFTVYNELTKVKYVTEGMRKPAFLSGEQKK |
| SV40NLS- | AIVDLLFKTNRKVTVKQLKEDYFKKIECFDSVEISGVEDRFNASLGTYHDLLKIIKDKDFLDNEENED |
| SGGSx2 | ILEDIVLTLTLFEDREMIEERLKTYAHLFDDKVMKQLKRRRYTGWGRLSRKLINGIRDKQSGKTILDF |
| (SEQ ID | LKSDGFANRNFMQLIHDDSLTFKEDIQKAQVSGQGDSLHEHIANLAGSPAIKKGILQTVKVVDELVKV |
| NO: | MGRHKPENIVIEMARENQTTQKGQKNSRERMKRIEEGIKELGSQILKEHPVENTQLQNEKLYLYYLQN |
| 572)- | GRDMYVDQELDINRLSDYDVDAIVPQSFLKDDSIDNKVLTRSDKNRGKSDNVPSEEVVKKMKNYWRQL |
| Klenow | LNAKLITQRKFDNLTKAERGGLSELDKAGFIKRQLVETRQITKHVAQILDSRMNTKYDENDKLIREVK |
| (E.coli)- | VITLKSKLVSDFRKDFQFYKVREINNYHHAHDAYLNAVVGTALIKKYPKLESEFVYGDYKVYDVRKMI |
| SV40NLS | AKSEQEIGKATAKYFFYSNIMNFFKTEITLANGEIRKRPLIETNGETGEIVWDKGRDFATVRKVLSMP |
| | QVNIVKKTEVQTGGFSKESILPKRNSDKLIARKKDWDPKKYGGFDSPTVAYSVLVVAKVEKGKSKKLK |
| | SVKELLGITIMERSSFEKNPIDFLEAKGYKEVKKDLIIKLPKYSLFELENGRKRMLASAGELQKGNEL |
| | ALPSKYVNFLYLASHYEKLKGSPEDNEQKQLFVEQHKHYLDEIIEQISEFSKRVILADANLDKVLSAY |
| | NKHRDKPIREQAENIIHLFTLTNLGAPAAFKYFDTTIDRKRYTSTKEVLDATLIHQSITGLYETRIDL |
| | SQLGGDSGGSSGGSKRTADGSEFESPKKKRKVSGGSSGGSVISYDNYVTILDEETLKAWIAKLEKAPV |
| | FAFATATDSLDNISANLVGLSFAIEPGVAAYIPVAHDYLDAPDQISRERALELLKPLLEDEKALKVGQ |
| | NLKYDRGILANYGIELRGIAFDTMLESYILNSVAGRHDMDSLAERWLKHKTITFEEIAGKGKNQLTFN |
| | QIALEEAGRYAAEDADVTLQLHLKMWPDLQKHKGPLNVFENIEMPLVPVLSRIERNGVKIDPKVLHNH |
| | SEELTLRLAELEKKAHEIAGEEFNLSSTKQLQTILFEKQGIKPLKKTPGGAPSTSEEVLEELALDYPL |
| | PKVILEYRGLAKLKSTYTDKLPLMINPKTGRVHTSYHQAVTATGRLSSTDPNLQNIPVRNEEGRRIRQ |
| | AFIAPEDYVIVSADYSQIELRIMAHLSRDKGLLTAFAEGKDIHRATAAEVFGLPLETVTSEQRRSAKA |
| | INFGLIYGMSAFGLARQLNIPRKEAQKYMDLYFERYPGVLEYMERTRAQAKEQGYVETLDGRRLYLPD |
| | IKSSNGARRAAAERAAINAPMQGTAADIIKRAMIAVDAWLQAEQPRVRMIMQVHDELVFEVHKDDVDA |
| | VAKQIHQLMENCTRLDVPLLVEVGSGENWDQAHSGGSKRTADGSEFESPKKKRKV (SEQ ID |
| | NO: 183) |
| pCMV- | MESKRKKRQNPAKRWCFTLNNYTQLEAITIEQLLCTEEVQYAIVGEEIGENGTPHLQGFFNLKKKKRL |
| T7- | TSLKAWLNDRAHYEEAKGSDEQNRRYCSKSGNILISFGSPQKQGQRSGGSSGSETPGTSESATPESSG |
| NG13- | GSDKKYSIGLDIGTNSVGWAVITDEYKVPSKKFKVLGNTDRHSIKKNLIGALLFDSGETAEATRLKRT |
| XTEN24- | ARRRYTRRKNRICYLQEIFSNEMAKVDDSFFHRLEESFLVEEDKKHERHPIFGNIVDEVAYHEKYPTI |
| nCas9 | YHLRKKLVDSTDKADLRLIYLALAHMIKFRGHFLIEGDLNPDNSDVDKLFIQLVQTYNQLFEENPINA |
| (H840A)- | SGVDAKAILSARLSKSRRLENLIAQLPGEKKNGLFGNLIALSLGLTPNFKSNFDLAEDAKLQLSKDTY |
| SGGSx2 | DDDLDNLLAQIGDQYADLFLAAKNLSDAILLSDILRVNTEITKAPLSASMIKRYDEHHQDLTLLKALV |
| (SEQ ID | RQQLPEKYKEIFFDQSKNGYAGYIDGGASQEEFYKFIKPILEKMDGTEELLVKLNREDLLRKQRTFDN |
| NO: | GSIPHQIHLGELHAILRRQEDFYPFLKDNREKIEKILTFRIPYYVGPLARGNSRFAWMTRKSEETITP |
| 572)- | WNFEEVVDKGASAQSFIERMTNFDKNLPNEKVLPKHSLLYEYFTVYNELTKVKYVTEGMRKPAFLSGE |
| SV40NLS- | QKKAIVDLLFKTNRKVTVKQLKEDYFKKIECFDSVEISGVEDRFNASLGTYHDLLKIIKDKDFLDNEE |
| SGGSx2 | NEDILEDIVLTLTLFEDREMIEERLKTYAHLFDDKVMKQLKRRRYTGWGRLSRKLINGIRDKQSGKTI |
| (SEQ ID | LDFLKSDGFANRNFMQLIHDDSLTFKEDIQKAQVSGQGDSLHEHIANLAGSPAIKKGILQTVKVVDEL |
| NO: | VKVMGRHKPENIVIEMARENQTTQKGQKNSRERMKRIEEGIKELGSQILKEHPVENTQLQNEKLYLYY |
| 572)- | LQNGRDMYVDQELDINRLSDYDVDAIVPQSFLKDDSIDNKVLTRSDKNRGKSDNVPSEEVVKKMKNYW |
| Klenow | RQLLNAKLITQRKFDNLTKAERGGLSELDKAGFIKRQLVETRQITKHVAQILDSRMNTKYDENDKLIR |
| (E.coli)- | EVKVITLKSKLVSDFRKDFQFYKVREINNYHHAHDAYLNAVVGTALIKKYPKLESEFVYGDYKVYDVR |
| SV40NLS | KMIAKSEQEIGKATAKYFFYSNIMNFFKTEITLANGEIRKRPLIETNGETGEIVWDKGRDFATVRKVL |
| | SMPQVNIVKKTEVQTGGFSKESILPKRNSDKLIARKKDWDPKKYGGFDSPTVAYSVLVVAKVEKGKSK |
| | KLKSVKELLGITIMERSSFEKNPIDFLEAKGYKEVKKDLIIKLPKYSLFELENGRKRMLASAGELQKG |
| | NELALPSKYVNFLYLASHYEKLKGSPEDNEQKQLFVEQHKHYLDEIIEQISEFSKRVILADANLDKVL |
| | SAYNKHRDKPIREQAENIIHLFTLTNLGAPAAFKYFDTTIDRKRYTSTKEVLDATLIHQSITGLYETR |
| | IDLSQLGGDSGGSSGGSKRTADGSEFESPKKKRKVSGGSSGGSVISYDNYVTILDEETLKAWIAKLEK |
| | APVFAFATATDSLDNISANLVGLSFAIEPGVAAYIPVAHDYLDAPDQISRERALELLKPLLEDEKALK |
| | VGQNLKYDRGILANYGIELRGIAFDTMLESYILNSVAGRHDMDSLAERWLKHKTITFEEIAGKGKNQL |
| | TFNQIALEEAGRYAAEDADVTLQLHLKMWPDLQKHKGPLNVFENIEMPLVPVLSRIERNGVKIDPKVL |
| | HNHSEELTLRLAELEKKAHEIAGEEFNLSSTKQLQTILFEKQGIKPLKKTPGGAPSTSEEVLEELALD |
| | YPLPKVILEYRGLAKLKSTYTDKLPLMINPKTGRVHTSYHQAVTATGRLSSTDPNLQNIPVRNEEGRR |
| | IRQAFIAPEDYVIVSADYSQIELRIMAHLSRDKGLLTAFAEGKDIHRATAAEVFGLPLETVTSEQRRS |
| | AKAINFGLIYGMSAFGLARQLNIPRKEAQKYMDLYFERYPGVLEYMERTRAQAKEQGYVETLDGRRLY |
| | LPDIKSSNGARRAAAERAAINAPMQGTAADIIKRAMIAVDAWLQAEQPRVRMIMQVHDELVFEVHKDD |
| | VDAVAKQIHQLMENCTRLDVPLLVEVGSGENWDQAHSGGSKRTADGSEFESPKKKRKV (SEQ ID |
| | NO: 184) |
| pCMV- | MPPQKREAAAKRWCFTLNNYTDEEVSAVKAWNASEYHYAVVGREKGENGTPHLQGYIHLKKKARLSTL |
| T7- | KKLLSRAHWEKARGSDSDNEAYCTKDGDVILTLGMPVEGNRSGGSSGSETPGTSESATPESSGGSDKK |
| RaCV- | YSIGLDIGTNSVGWAVITDEYKVPSKKFKVLGNTDRHSIKKNLIGALLFDSGETAEATRLKRTARRRY |
| XTEN24- | TRRKNRICYLQEIFSNEMAKVDDSFFHRLEESFLVEEDKKHERHPIFGNIVDEVAYHEKYPTIYHLRK |
| nCas9 | KLVDSTDKADLRLIYLALAHMIKFRGHFLIEGDLNPDNSDVDKLFIQLVQTYNQLFEENPINASGVDA |
| (H840A)- | KAILSARLSKSRRLENLIAQLPGEKKNGLFGNLIALSLGLTPNFKSNFDLAEDAKLQLSKDTYDDDLD |
| SGGSx2 | NLLAQIGDQYADLFLAAKNLSDAILLSDILRVNTEITKAPLSASMIKRYDEHHQDLTLLKALVRQQLP |
| (SEQ ID | EKYKEIFFDQSKNGYAGYIDGGASQEEFYKFIKPILEKMDGTEELLVKLNREDLLRKQRTFDNGSIPH |
| NO: | QIHLGELHAILRRQEDFYPFLKDNREKIEKILTFRIPYYVGPLARGNSRFAWMTRKSEETITPWNFEE |
| 572)- | VVDKGASAQSFIERMTNFDKNLPNEKVLPKHSLLYEYFTVYNELTKVKYVTEGMRKPAFLSGEQKKAI |
| SV40NLS- | VDLLFKTNRKVTVKQLKEDYFKKIECFDSVEISGVEDRFNASLGTYHDLLKIIKDKDFLDNEENEDIL |
| SGGSx2 | EDIVLTLTLFEDREMIEERLKTYAHLFDDKVMKQLKRRRYTGWGRLSRKLINGIRDKQSGKTILDFLK |
| (SEQ ID | SDGFANRNFMQLIHDDSLTFKEDIQKAQVSGQGDSLHEHIANLAGSPAIKKGILQTVKVVDELVKVMG |
| NO: | RHKPENIVIEMARENQTTQKGQKNSRERMKRIEEGIKELGSQILKEHPVENTQLQNEKLYLYYLQNGR |
| 572)- | DMYVDQELDINRLSDYDVDAIVPQSFLKDDSIDNKVLTRSDKNRGKSDNVPSEEVVKKMKNYWRQLLN |
| Klenow | AKLITQRKFDNLTKAERGGLSELDKAGFIKRQLVETRQITKHVAQILDSRMNTKYDENDKLIREVKVI |
| (E.coli)- | TLKSKLVSDFRKDFQFYKVREINNYHHAHDAYLNAVVGTALIKKYPKLESEFVYGDYKVYDVRKMIAK |
| SV40NLS | SEQEIGKATAKYFFYSNIMNFFKTEITLANGEIRKRPLIETNGETGEIVWDKGRDFATVRKVLSMPQV |
| | NIVKKTEVQTGGFSKESILPKRNSDKLIARKKDWDPKKYGGFDSPTVAYSVLVVAKVEKGKSKKLKSV |
| | KELLGITIMERSSFEKNPIDFLEAKGYKEVKKDLIIKLPKYSLFELENGRKRMLASAGELQKGNELAL |
| | PSKYVNFLYLASHYEKLKGSPEDNEQKQLFVEQHKHYLDEIIEQISEFSKRVILADANLDKVLSAYNK |
| | HRDKPIREQAENIIHLFTLTNLGAPAAFKYFDTTIDRKRYTSTKEVLDATLIHQSITGLYETRIDLSQ |
| | LGGDSGGSSGGSKRTADGSEFESPKKKRKVSGGSSGGSVISYDNYVTILDEETLKAWIAKLEKAPVFA |
| | FATATDSLDNISANLVGLSFAIEPGVAAYIPVAHDYLDAPDQISRERALELLKPLLEDEKALKVGQNL |
| | KYDRGILANYGIELRGIAFDTMLESYILNSVAGRHDMDSLAERWLKHKTITFEEIAGKGKNQLTFNQI |
| | ALEEAGRYAAEDADVTLQLHLKMWPDLQKHKGPLNVFENIEMPLVPVLSRIERNGVKIDPKVLHNHSE |
| | ELTLRLAELEKKAHEIAGEEFNLSSTKQLQTILFEKQGIKPLKKTPGGAPSTSEEVLEELALDYPLPK |
| | VILEYRGLAKLKSTYTDKLPLMINPKTGRVHTSYHQAVTATGRLSSTDPNLQNIPVRNEEGRRIRQAF |
| | IAPEDYVIVSADYSQIELRIMAHLSRDKGLLTAFAEGKDIHRATAAEVFGLPLETVTSEQRRSAKAIN |
| | FGLIYGMSAFGLARQLNIPRKEAQKYMDLYFERYPGVLEYMERTRAQAKEQGYVETLDGRRLYLPDIK |
| | SSNGARRAAAERAAINAPMQGTAADIIKRAMIAVDAWLQAEQPRVRMIMQVHDELVFEVHKDDVDAVA |
| | KQIHQLMENCTRLDVPLLVEVGSGENWDQAHSGGSKRTADGSEFESPKKKRKV (SEQ ID |
| | NO: 185) |
| pCMV- | MRMSKMARKDPTIEFCQLGLDTFETKYITMFGMLVSCSFDKPAFISFVFSDFTKNDIVQNYLYDRYLI |
| T7 | DYENKLELNEGFKAIMYKNQFETFDSKLRKIFNNGLRDLQNGRDENLSQYGIVCKMNIKVKMYNGKLN |
| Cdc13- | AIVRECEPVPHSQISSIASPSQCEHLRLFYQRAFKRIGESAISRYFEEYRRFFPIHRNGSHLASGGSS |
| XTEN24- | GSETPGTSESATPESSGGSDKKYSIGLDIGTNSVGWAVITDEYKVPSKKFKVLGNTDRHSIKKNLIGA |
| nCas9 | LLFDSGETAEATRLKRTARRRYTRRKNRICYLQEIFSNEMAKVDDSFFHRLEESFLVEEDKKHERHPI |
| (H840A)- | FGNIVDEVAYHEKYPTIYHLRKKLVDSTDKADLRLIYLALAHMIKFRGHFLIEGDLNPDNSDVDKLFI |
| SGGSx2 | QLVQTYNQLFEENPINASGVDAKAILSARLSKSRRLENLIAQLPGEKKNGLFGNLIALSLGLTPNFKS |
| (SEQ ID | NFDLAEDAKLQLSKDTYDDDLDNLLAQIGDQYADLFLAAKNLSDAILLSDILRVNTEITKAPLSASMI |
| NO: | KRYDEHHQDLTLLKALVRQQLPEKYKEIFFDQSKNGYAGYIDGGASQEEFYKFIKPILEKMDGTEELL |
| 572)- | VKLNREDLLRKQRTFDNGSIPHQIHLGELHAILRRQEDFYPFLKDNREKIEKILTFRIPYYVGPLARG |
| SV40NLS- | NSRFAWMTRKSEETITPWNFEEVVDKGASAQSFIERMTNFDKNLPNEKVLPKHSLLYEYFTVYNELTK |
| SGGSx2 | VKYVTEGMRKPAFLSGEQKKAIVDLLFKTNRKVTVKQLKEDYFKKIECFDSVEISGVEDRFNASLGTY |
| (SEQ ID | HDLLKIIKDKDFLDNEENEDILEDIVLTLTLFEDREMIEERLKTYAHLFDDKVMKQLKRRRYTGWGRL |
| NO: | SRKLINGIRDKQSGKTILDFLKSDGFANRNFMQLIHDDSLTFKEDIQKAQVSGQGDSLHEHIANLAGS |
| 572)- | PAIKKGILQTVKVVDELVKVMGRHKPENIVIEMARENQTTQKGQKNSRERMKRIEEGIKELGSQILKE |
| Klenow | HPVENTQLQNEKLYLYYLQNGRDMYVDQELDINRLSDYDVDAIVPQSFLKDDSIDNKVLTRSDKNRGK |
| (E.coli)- | SDNVPSEEVVKKMKNYWRQLLNAKLITQRKFDNLTKAERGGLSELDKAGFIKRQLVETRQITKHVAQI |
| SV40NLS | LDSRMNTKYDENDKLIREVKVITLKSKLVSDFRKDFQFYKVREINNYHHAHDAYLNAVVGTALIKKYP |
| | KLESEFVYGDYKVYDVRKMIAKSEQEIGKATAKYFFYSNIMNFFKTEITLANGEIRKRPLIETNGETG |
| | EIVWDKGRDFATVRKVLSMPQVNIVKKTEVQTGGFSKESILPKRNSDKLIARKKDWDPKKYGGFDSPT |
| | VAYSVLVVAKVEKGKSKKLKSVKELLGITIMERSSFEKNPIDFLEAKGYKEVKKDLIIKLPKYSLFEL |
| | ENGRKRMLASAGELQKGNELALPSKYVNFLYLASHYEKLKGSPEDNEQKQLFVEQHKHYLDEIIEQIS |
| | EFSKRVILADANLDKVLSAYNKHRDKPIREQAENIIHLFTLTNLGAPAAFKYFDTTIDRKRYTSTKEV |
| | LDATLIHQSITGLYETRIDLSQLGGDSGGSSGGSKRTADGSEFESPKKKRKVSGGSSGGSVISYDNYV |
| | TILDEETLKAWIAKLEKAPVFAFATATDSLDNISANLVGLSFAIEPGVAAYIPVAHDYLDAPDQISRE |
| | RALELLKPLLEDEKALKVGQNLKYDRGILANYGIELRGIAFDTMLESYILNSVAGRHDMDSLAERWLK |
| | HKTITFEEIAGKGKNQLTFNQIALEEAGRYAAEDADVTLQLHLKMWPDLQKHKGPLNVFENIEMPLVP |
| | VLSRIERNGVKIDPKVLHNHSEELTLRLAELEKKAHEIAGEEFNLSSTKQLQTILFEKQGIKPLKKTP |
| | GGAPSTSEEVLEELALDYPLPKVILEYRGLAKLKSTYTDKLPLMINPKTGRVHTSYHQAVTATGRLSS |
| | TDPNLQNIPVRNEEGRRIRQAFIAPEDYVIVSADYSQIELRIMAHLSRDKGLLTAFAEGKDIHRATAA |
| | EVFGLPLETVTSEQRRSAKAINFGLIYGMSAFGLARQLNIPRKEAQKYMDLYFERYPGVLEYMERTRA |
| | QAKEQGYVETLDGRRLYLPDIKSSNGARRAAAERAAINAPMQGTAADIIKRAMIAVDAWLQAEQPRVR |
| | MIMQVHDELVFEVHKDDVDAVAKQIHQLMENCTRLDVPLLVEVGSGENWDQAHSGGSKRTADGSEFES |
| | PKKKRKV (SEQ ID NO: 186) |
| pCMV- | MRMSKMARKDPTIEFCQLGLDTFETKYITMFGMLVSCSFDKPAFISFVFSDFTKNDIVQNALYDRYLI |
| T7- | DYENKLELNEGFKAIMYKNQFETFDSKLRKIFNNGLRDLQNGRDENLSQYGIVCKMNIKVKMYNGKLN |
| Cdc13 | AIVRECEPVPHSQISSIASPSQCEHLRLFYQRAFKRIGESAISRYFEEYRRFFPIHRNGSHLASGGSS |
| (Y556A)- | GSETPGTSESATPESSGGSDKKYSIGLDIGTNSVGWAVITDEYKVPSKKFKVLGNTDRHSIKKNLIGA |
| XTEN24- | LLFDSGETAEATRLKRTARRRYTRRKNRICYLQEIFSNEMAKVDDSFFHRLEESFLVEEDKKHERHPI |
| nCas9 | FGNIVDEVAYHEKYPTIYHLRKKLVDSTDKADLRLIYLALAHMIKFRGHFLIEGDLNPDNSDVDKLFI |
| (H840A)- | QLVQTYNQLFEENPINASGVDAKAILSARLSKSRRLENLIAQLPGEKKNGLFGNLIALSLGLTPNFKS |
| SGGSx2 | NFDLAEDAKLQLSKDTYDDDLDNLLAQIGDQYADLFLAAKNLSDAILLSDILRVNTEITKAPLSASMI |
| (SEQ ID | KRYDEHHQDLTLLKALVRQQLPEKYKEIFFDQSKNGYAGYIDGGASQEEFYKFIKPILEKMDGTEELL |
| NO: | VKLNREDLLRKQRTFDNGSIPHQIHLGELHAILRRQEDFYPFLKDNREKIEKILTFRIPYYVGPLARG |
| 572)- | NSRFAWMTRKSEETITPWNFEEVVDKGASAQSFIERMTNFDKNLPNEKVLPKHSLLYEYFTVYNELTK |
| SV40NLS- | VKYVTEGMRKPAFLSGEQKKAIVDLLFKTNRKVTVKQLKEDYFKKIECFDSVEISGVEDRFNASLGTY |
| SGGSx2 | HDLLKIIKDKDFLDNEENEDILEDIVLTLTLFEDREMIEERLKTYAHLFDDKVMKQLKRRRYTGWGRL |
| (SEQ ID | SRKLINGIRDKQSGKTILDFLKSDGFANRNFMQLIHDDSLTFKEDIQKAQVSGQGDSLHEHIANLAGS |
| NO: | PAIKKGILQTVKVVDELVKVMGRHKPENIVIEMARENQTTQKGQKNSRERMKRIEEGIKELGSQILKE |
| 572)- | HPVENTQLQNEKLYLYYLQNGRDMYVDQELDINRLSDYDVDAIVPQSFLKDDSIDNKVLTRSDKNRGK |
| Klenow | SDNVPSEEVVKKMKNYWRQLLNAKLITQRKFDNLTKAERGGLSELDKAGFIKRQLVETRQITKHVAQI |
| (E.coli)- | LDSRMNTKYDENDKLIREVKVITLKSKLVSDFRKDFQFYKVREINNYHHAHDAYLNAVVGTALIKKYP |
| SV40NLS | KLESEFVYGDYKVYDVRKMIAKSEQEIGKATAKYFFYSNIMNFFKTEITLANGEIRKRPLIETNGETG |
| | EIVWDKGRDFATVRKVLSMPQVNIVKKTEVQTGGFSKESILPKRNSDKLIARKKDWDPKKYGGFDSPT |
| | VAYSVLVVAKVEKGKSKKLKSVKELLGITIMERSSFEKNPIDFLEAKGYKEVKKDLIIKLPKYSLFEL |
| | ENGRKRMLASAGELQKGNELALPSKYVNFLYLASHYEKLKGSPEDNEQKQLFVEQHKHYLDEIIEQIS |
| | EFSKRVILADANLDKVLSAYNKHRDKPIREQAENIIHLFTLTNLGAPAAFKYFDTTIDRKRYTSTKEV |
| | LDATLIHQSITGLYETRIDLSQLGGDSGGSSGGSKRTADGSEFESPKKKRKVSGGSSGGSVISYDNYV |
| | TILDEETLKAWIAKLEKAPVFAFATATDSLDNISANLVGLSFAIEPGVAAYIPVAHDYLDAPDQISRE |
| | RALELLKPLLEDEKALKVGQNLKYDRGILANYGIELRGIAFDTMLESYILNSVAGRHDMDSLAERWLK |
| | HKTITFEEIAGKGKNQLTFNQIALEEAGRYAAEDADVTLQLHLKMWPDLQKHKGPLNVFENIEMPLVP |
| | VLSRIERNGVKIDPKVLHNHSEELTLRLAELEKKAHEIAGEEFNLSSTKQLQTILFEKQGIKPLKKTP |
| | GGAPSTSEEVLEELALDYPLPKVILEYRGLAKLKSTYTDKLPLMINPKTGRVHTSYHQAVTATGRLSS |
| | TDPNLQNIPVRNEEGRRIRQAFIAPEDYVIVSADYSQIELRIMAHLSRDKGLLTAFAEGKDIHRATAA |
| | EVFGLPLETVTSEQRRSAKAINFGLIYGMSAFGLARQLNIPRKEAQKYMDLYFERYPGVLEYMERTRA |
| | QAKEQGYVETLDGRRLYLPDIKSSNGARRAAAERAAINAPMQGTAADIIKRAMIAVDAWLQAEQPRVR |
| | MIMQVHDELVFEVHKDDVDAVAKQIHQLMENCTRLDVPLLVEVGSGENWDQAHSGGSKRTADGSEFES |
| | PKKKRKV (SEQ ID NO: 187) |
| LTR- | MSPSKKNGRSGPQPHKRWVFTLNNPSEDERKKIRDLPISLFDYFIVGEEGNEEGRTPHLQGFANFVKK |
| pEF1a- | QTFNKVKWYLGARCHIEKAKGTDQQNKEYCSKEGNLLMECGAPRSQGQRSGGSSGSETPGTSESATPE |
| PCV2- | SSGGSDKKYSIGLDIGTNSVGWAVITDEYKVPSKKFKVLGNTDRHSIKKNLIGALLFDSGETAEATRL |
| nCas9 | KRTARRRYTRRKNRICYLQEIFSNEMAKVDDSFFHRLEESFLVEEDKKHERHPIFGNIVDEVAYHEKY |
| (H840A)- | PTIYHLRKKLVDSTDKADLRLIYLALAHMIKFRGHFLIEGDLNPDNSDVDKLFIQLVQTYNQLFEENP |
| Ecklenow | INASGVDAKAILSARLSKSRRLENLIAQLPGEKKNGLFGNLIALSLGLTPNFKSNFDLAEDAKLQLSK |
| (-exo; | DTYDDDLDNLLAQIGDQYADLFLAAKNLSDAILLSDILRVNTEITKAPLSASMIKRYDEHHQDLTLLK |
| D355A/E357A)- | ALVRQQLPEKYKEIFFDQSKNGYAGYIDGGASQEEFYKFIKPILEKMDGTEELLVKLNREDLLRKQRT |
| P2A- | FDNGSIPHQIHLGELHAILRRQEDFYPFLKDNREKIEKILTFRIPYYVGPLARGNSRFAWMTRKSEET |
| PuroR | ITPWNFEEVVDKGASAQSFIERMTNFDKNLPNEKVLPKHSLLYEYFTVYNELTKVKYVTEGMRKPAFL |
| | SGEQKKAIVDLLFKTNRKVTVKQLKEDYFKKIECFDSVEISGVEDRFNASLGTYHDLLKIIKDKDFLD |
| | NEENEDILEDIVLTLTLFEDREMIEERLKTYAHLFDDKVMKQLKRRRYTGWGRLSRKLINGIRDKQSG |
| | KTILDFLKSDGFANRNFMQLIHDDSLTFKEDIQKAQVSGQGDSLHEHIANLAGSPAIKKGILQTVKVV |
| | DELVKVMGRHKPENIVIEMARENQTTQKGQKNSRERMKRIEEGIKELGSQILKEHPVENTQLQNEKLY |
| | LYYLQNGRDMYVDQELDINRLSDYDVDAIVPQSFLKDDSIDNKVLTRSDKNRGKSDNVPSEEVVKKMK |
| | NYWRQLLNAKLITQRKFDNLTKAERGGLSELDKAGFIKRQLVETRQITKHVAQILDSRMNTKYDENDK |
| | LIREVKVITLKSKLVSDFRKDFQFYKVREINNYHHAHDAYLNAVVGTALIKKYPKLESEFVYGDYKVY |
| | DVRKMIAKSEQEIGKATAKYFFYSNIMNFFKTEITLANGEIRKRPLIETNGETGEIVWDKGRDFATVR |
| | KVLSMPQVNIVKKTEVQTGGFSKESILPKRNSDKLIARKKDWDPKKYGGFDSPTVAYSVLVVAKVEKG |
| | KSKKLKSVKELLGITIMERSSFEKNPIDFLEAKGYKEVKKDLIIKLPKYSLFELENGRKRMLASAGEL |
| | QKGNELALPSKYVNFLYLASHYEKLKGSPEDNEQKQLFVEQHKHYLDEIIEQISEFSKRVILADANLD |
| | KVLSAYNKHRDKPIREQAENIIHLFTLTNLGAPAAFKYFDTTIDRKRYTSTKEVLDATLIHQSITGLY |
| | ETRIDLSQLGGDSGGSSGGSKRTADGSEFESPKKKRKVSGGSSGGSVISYDNYVTILDEETLKAWIAK |
| | LEKAPVFAFATATDSLDNISANLVGLSFAIEPGVAAYIPVAHDYLDAPDQISRERALELLKPLLEDEK |
| | ALKVGQNLKYDRGILANYGIELRGIAFDTMLESYILNSVAGRHDMDSLAERWLKHKTITFEEIAGKGK |
| | NQLTFNQIALEEAGRYAAEDADVTLQLHLKMWPDLQKHKGPLNVFENIEMPLVPVLSRIERNGVKIDP |
| | KVLHNHSEELTLRLAELEKKAHEIAGEEFNLSSTKQLQTILFEKQGIKPLKKTPGGAPSTSEEVLEEL |
| | ALDYPLPKVILEYRGLAKLKSTYTDKLPLMINPKTGRVHTSYHQAVTATGRLSSTDPNLQNIPVRNEE |
| | GRRIRQAFIAPEDYVIVSADYSQIELRIMAHLSRDKGLLTAFAEGKDIHRATAAEVFGLPLETVTSEQ |
| | RRSAKAINFGLIYGMSAFGLARQLNIPRKEAQKYMDLYFERYPGVLEYMERTRAQAKEQGYVETLDGR |
| | RLYLPDIKSSNGARRAAAERAAINAPMQGTAADIIKRAMIAVDAWLQAEQPRVRMIMQVHDELVFEVH |
| | KDDVDAVAKQIHQLMENCTRLDVPLLVEVGSGENWDQAHSGGSKRTADGSEFESPKKKRKVGSGATNF |
| | SLLKQAGDVEENPGPTEYKPTVRLATRDDVPRAVRTLAAAFADYPATRHTVDPDRHIERVTELQELFL |
| | TRVGLDIGKVWVADDGAAVAVWTTPESVEAGAVFAEIGPRMAELSGSRLAAQQQMEGLLAPHRPKEPA |
| | WFLATVGVSPDHQGKGLGSAVVLPGVEAAERAGVPAFLETSAPRNLPFYERLGFTVTADVEVPEGPRT |
| | WCMTRKPGA (SEQ ID NO: 188) |
| pCMV- | MSPSKKNGRSGPQPHKRWVFTLNNPSEDERKKIRDLPISLFDYFIVGEEGNEEGRTPHLQGFANFVKK |
| T7- | QTFNKVKWYLGARCHIEKAKGTDQQNKEYCSKEGNLLMECGAPRSQGQRSGGSSGSETPGTSESATPE |
| PCV2- | SSGGSDKKYSIGLDIGTNSVGWAVITDEYKVPSKKFKVLGNTDRHSIKKNLIGALLFDSGETAEATRL |
| nCas9 | KRTARRRYTRRKNRICYLQEIFSNEMAKVDDSFFHRLEESFLVEEDKKHERHPIFGNIVDEVAYHEKY |
| (H840A)- | PTIYHLRKKLVDSTDKADLRLIYLALAHMIKFRGHFLIEGDLNPDNSDVDKLFIQLVQTYNQLFEENP |
| M- | INASGVDAKAILSARLSKSRRLENLIAQLPGEKKNGLFGNLIALSLGLTPNFKSNFDLAEDAKLQLSK |
| MLV (PE1) | DTYDDDLDNLLAQIGDQYADLFLAAKNLSDAILLSDILRVNTEITKAPLSASMIKRYDEHHQDLTLLK |
| | ALVRQQLPEKYKEIFFDQSKNGYAGYIDGGASQEEFYKFIKPILEKMDGTEELLVKLNREDLLRKQRT |
| | FDNGSIPHQIHLGELHAILRRQEDFYPFLKDNREKIEKILTFRIPYYVGPLARGNSRFAWMTRKSEET |
| | ITPWNFEEVVDKGASAQSFIERMTNFDKNLPNEKVLPKHSLLYEYFTVYNELTKVKYVTEGMRKPAFL |
| | SGEQKKAIVDLLFKTNRKVTVKQLKEDYFKKIECFDSVEISGVEDRFNASLGTYHDLLKIIKDKDFLD |
| | NEENEDILEDIVLTLTLFEDREMIEERLKTYAHLFDDKVMKQLKRRRYTGWGRLSRKLINGIRDKQSG |
| | KTILDFLKSDGFANRNFMQLIHDDSLTFKEDIQKAQVSGQGDSLHEHIANLAGSPAIKKGILQTVKVV |
| | DELVKVMGRHKPENIVIEMARENQTTQKGQKNSRERMKRIEEGIKELGSQILKEHPVENTQLQNEKLY |
| | LYYLQNGRDMYVDQELDINRLSDYDVDAIVPQSFLKDDSIDNKVLTRSDKNRGKSDNVPSEEVVKKMK |
| | NYWRQLLNAKLITQRKFDNLTKAERGGLSELDKAGFIKRQLVETRQITKHVAQILDSRMNTKYDENDK |
| | LIREVKVITLKSKLVSDFRKDFQFYKVREINNYHHAHDAYLNAVVGTALIKKYPKLESEFVYGDYKVY |
| | DVRKMIAKSEQEIGKATAKYFFYSNIMNFFKTEITLANGEIRKRPLIETNGETGEIVWDKGRDFATVR |
| | KVLSMPQVNIVKKTEVQTGGFSKESILPKRNSDKLIARKKDWDPKKYGGFDSPTVAYSVLVVAKVEKG |
| | KSKKLKSVKELLGITIMERSSFEKNPIDFLEAKGYKEVKKDLIIKLPKYSLFELENGRKRMLASAGEL |
| | QKGNELALPSKYVNFLYLASHYEKLKGSPEDNEQKQLFVEQHKHYLDEIIEQISEFSKRVILADANLD |
| | KVLSAYNKHRDKPIREQAENIIHLFTLTNLGAPAAFKYFDTTIDRKRYTSTKEVLDATLIHQSITGLY |
| | ETRIDLSQLGGDSGGSSGGSSGSETPGTSESATPESSGGSSGGSSTLNIEDEYRLHETSKEPDVSLGS |
| | TWLSDFPQAWAETGGMGLAVRQAPLIIPLKATSTPVSIKQYPMSQEARLGIKPHIQRLLDQGILVPCQ |
| | SPWNTPLLPVKKPGTNDYRPVQDLREVNKRVEDIHPTVPNPYNLLSGLPPSHQWYTVLDLKDAFFCLR |
| | LHPTSQPLFAFEWRDPEMGISGQLTWTRLPQGFKNSPTLFDEALHRDLADFRIQHPDLILLQYVDDLL |
| | LAATSELDCQQGTRALLQTLGNLGYRASAKKAQICQKQVKYLGYLLKEGQRWLTEARKETVMGQPTPK |
| | TPRQLREFLGTAGFCRLWIPGFAEMAAPLYPLTKTGTLFNWGPDQQKAYQEIKQALLTAPALGLPDLT |
| | KPFELFVDEKQGYAKGVLTQKLGPWRRPVAYLSKKLDPVAAGWPPCLRMVAAIAVLTKDAGKLTMGQP |
| | LVILAPHAVEALVKQPPDRWLSNARMTHYQALLLDTDRVQFGPVVALNPATLLPLPEEGLQHNCLDIL |
| | AEAHGTRPDLTDQPLPDADHTWYTDGSSLLQEGQRKAGAAVTTETEVIWAKALPAGTSAQRAELIALT |
| | QALKMAEGKKLNVYTDSRYAFATAHIHGEIYRRRGLLTSEGKEIKNKDEILALLKALFLPKRLSIIHC |
| | PGHQKGHSAEARGNRMADQAARKAAITETPDTSTLLIENSSPSGGSKRTADGSEFEPKKKRKV |
| | (SEQ ID NO: 189) |
| pCMV- | MSPSKKNGRSGPQPHKRWVFTLNNPSEDERKKIRDLPISLFDYFIVGEEGNEEGRTPHLQGFANFVKK |
| T7- | QTFNKVKWYLGARCHIEKAKGTDQQNKEYCSKEGNLLMECGAPRSQGQRSGGSSGSETPGTSESATPE |
| PCV2- | SSGGSGKRNYILGLDIGITSVGYGIIDYETRDVIDAGVRLFKEANVENNEGRRSKRGARRLKRRRRHR |
| nSaCas9 | IQRVKKLLFDYNLLTDHSELSGINPYEARVKGLSQKLSEEEFSAALLHLAKRRGVHNVNEVEEDTGNE |
| (N580A)- | LSTKEQISRNSKALEEKYVAELQLERLKKDGEVRGSINRFKTSDYVKEAKQLLKVQKAYHQLDQSFID |
| Ecklenow | TYIDLLETRRTYYEGPGEGSPFGWKDIKEWYEMLMGHCTYFPEELRSVKYAYNADLYNALNDLNNLVI |
| (-exo; D355A/ | TRDENEKLEYYEKFQIIENVFKQKKKPTLKQIAKEILVNEEDIKGYRVTSTGKPEFTNLKVYHDIKDI |
| E357A) | TARKEIIENAELLDQIAKILTIYQSSEDIQEELTNLNSELTQEEIEQISNLKGYTGTHNLSLKAINLI |
| | LDELWHTNDNQIAIFNRLKLVPKKVDLSQQKEIPTTLVDDFILSPVVKRSFIQSIKVINAIIKKYGLP |
| | NDIIIELAREKNSKDAQKMINEMQKRNRQTNERIEEIIRTTGKENAKYLIEKIKLHDMQEGKCLYSLE |
| | AIPLEDLLNNPFNYEVDHIIPRSVSFDNSFNNKVLVKQEEASKKGNRTPFQYLSSSDSKISYETFKKH |
| | ILNLAKGKGRISKTKKEYLLEERDINRFSVQKDFINRNLVDTRYATRGLMNLLRSYFRVNNLDVKVKS |
| | INGGFTSFLRRKWKFKKERNKGYKHHAEDALIIANADFIFKEWKKLDKAKKVMENQMFEEKQAESMPE |
| | IETEQEYKEIFITPHQIKHIKDFKDYKYSHRVDKKPNRELINDTLYSTRKDDKGNTLIVNNLNGLYDK |
| | DNDKLKKLINKSPEKLLMYHHDPQTYQKLKLIMEQYGDEKNPLYKYYEETGNYLTKYSKKDNGPVIKK |
| | IKYYGNKLNAHLDITDDYPNSRNKVVKLSLKPYRFDVYLDNGVYKFVTVKNLDVIKKENYYEVNSKCY |
| | EEAKKLKKISNQAEFIASFYNNDLIKINGELYRVIGVNNDLLNRIEVNMIDITYREYLENMNDKRPPR |
| | IIKTIASKTQSIKKYSTDILGNLYEVKSKKHPQIIKKGSGGSSGGSKRTADGSEFESPKKKRKVSGGS |
| | SGGSVISYDNYVTILDEETLKAWIAKLEKAPVFAFATATDSLDNISANLVGLSFAIEPGVAAYIPVAH |
| | DYLDAPDQISRERALELLKPLLEDEKALKVGQNLKYDRGILANYGIELRGIAFDTMLESYILNSVAGR |
| | HDMDSLAERWLKHKTITFEEIAGKGKNQLTFNQIALEEAGRYAAEDADVTLQLHLKMWPDLQKHKGPL |
| | NVFENIEMPLVPVLSRIERNGVKIDPKVLHNHSEELTLRLAELEKKAHEIAGEEFNLSSTKQLQTILF |
| | EKQGIKPLKKTPGGAPSTSEEVLEELALDYPLPKVILEYRGLAKLKSTYTDKLPLMINPKTGRVHTSY |
| | HQAVTATGRLSSTDPNLQNIPVRNEEGRRIRQAFIAPEDYVIVSADYSQIELRIMAHLSRDKGLLTAF |
| | AEGKDIHRATAAEVFGLPLETVTSEQRRSAKAINFGLIYGMSAFGLARQLNIPRKEAQKYMDLYFERY |
| | PGVLEYMERTRAQAKEQGYVETLDGRRLYLPDIKSSNGARRAAAERAAINAPMQGTAADIIKRAMIAV |
| | DAWLQAEQPRVRMIMQVHDELVFEVHKDDVDAVAKQIHQLMENCTRLDVPLLVEVGSGENWDQAHSGG |
| | SKRTADGSEFESPKKKRKV (SEQ ID NO: 190) |
| pCMV- | MGAQPARSMAEAGITGTWYNQSGSTFTVTAGADGNLTGQYENRAQGTGCQNSPYTLTGRYNGTKLEWR |
| T7-mSA- | VEWNNSTENCHSRTEWRGQYQGGAEARINTQWNLTYEGGSGPATEQGQDTFTKVKPSAASGSSAGGGG |
| nSpCas9 | SGGGGSGGGGSGPKKKRKVAAAGSDKKYSIGLDIGTNSVGWAVITDEYKVPSKKFKVLGNTDRHSIKK |
| (H840A)- | NLIGALLFDSGETAEATRLKRTARRRYTRRKNRICYLQEIFSNEMAKVDDSFFHRLEESFLVEEDKKH |
| Ecklenow | ERHPIFGNIVDEVAYHEKYPTIYHLRKKLVDSTDKADLRLIYLALAHMIKFRGHFLIEGDLNPDNSDV |
| (-exo; D355A/ | DKLFIQLVQTYNQLFEENPINASGVDAKAILSARLSKSRRLENLIAQLPGEKKNGLFGNLIALSLGLT |
| E357A) | PNFKSNFDLAEDAKLQLSKDTYDDDLDNLLAQIGDQYADLFLAAKNLSDAILLSDILRVNTEITKAPL |
| | SASMIKRYDEHHQDLTLLKALVRQQLPEKYKEIFFDQSKNGYAGYIDGGASQEEFYKFIKPILEKMDG |
| | TEELLVKLNREDLLRKQRTFDNGSIPHQIHLGELHAILRRQEDFYPFLKDNREKIEKILTFRIPYYVG |
| | PLARGNSRFAWMTRKSEETITPWNFEEVVDKGASAQSFIERMTNFDKNLPNEKVLPKHSLLYEYFTVY |
| | NELTKVKYVTEGMRKPAFLSGEQKKAIVDLLFKTNRKVTVKQLKEDYFKKIECFDSVEISGVEDRFNA |
| | SLGTYHDLLKIIKDKDFLDNEENEDILEDIVLTLTLFEDREMIEERLKTYAHLFDDKVMKQLKRRRYT |
| | GWGRLSRKLINGIRDKQSGKTILDFLKSDGFANRNFMQLIHDDSLTFKEDIQKAQVSGQGDSLHEHIA |
| | NLAGSPAIKKGILQTVKVVDELVKVMGRHKPENIVIEMARENQTTQKGQKNSRERMKRIEEGIKELGS |
| | QILKEHPVENTQLQNEKLYLYYLQNGRDMYVDQELDINRLSDYDVDAIVPQSFLKDDSIDNKVLTRSD |
| | KNRGKSDNVPSEEVVKKMKNYWRQLLNAKLITQRKFDNLTKAERGGLSELDKAGFIKRQLVETRQITK |
| | HVAQILDSRMNTKYDENDKLIREVKVITLKSKLVSDFRKDFQFYKVREINNYHHAHDAYLNAVVGTAL |
| | IKKYPKLESEFVYGDYKVYDVRKMIAKSEQEIGKATAKYFFYSNIMNFFKTEITLANGEIRKRPLIET |
| | NGETGEIVWDKGRDFATVRKVLSMPQVNIVKKTEVQTGGFSKESILPKRNSDKLIARKKDWDPKKYGG |
| | FDSPTVAYSVLVVAKVEKGKSKKLKSVKELLGITIMERSSFEKNPIDFLEAKGYKEVKKDLIIKLPKY |
| | SLFELENGRKRMLASAGELQKGNELALPSKYVNFLYLASHYEKLKGSPEDNEQKQLFVEQHKHYLDEI |
| | IEQISEFSKRVILADANLDKVLSAYNKHRDKPIREQAENIIHLFTLTNLGAPAAFKYFDTTIDRKRYT |
| | STKEVLDATLIHQSITGLYETRIDLSQLGGDSGGSSGGSKRTADGSEFESPKKKRKVSGGSSGGSVIS |
| | YDNYVTILDEETLKAWIAKLEKAPVFAFATATDSLDNISANLVGLSFAIEPGVAAYIPVAHDYLDAPD |
| | QISRERALELLKPLLEDEKALKVGQNLKYDRGILANYGIELRGIAFDTMLESYILNSVAGRHDMDSLA |
| | ERWLKHKTITFEEIAGKGKNQLTFNQIALEEAGRYAAEDADVTLQLHLKMWPDLQKHKGPLNVFENIE |
| | MPLVPVLSRIERNGVKIDPKVLHNHSEELTLRLAELEKKAHEIAGEEFNLSSTKQLQTILFEKQGIKP |
| | LKKTPGGAPSTSEEVLEELALDYPLPKVILEYRGLAKLKSTYTDKLPLMINPKTGRVHTSYHQAVTAT |
| | GRLSSTDPNLQNIPVRNEEGRRIRQAFIAPEDYVIVSADYSQIELRIMAHLSRDKGLLTAFAEGKDIH |
| | RATAAEVFGLPLETVTSEQRRSAKAINFGLIYGMSAFGLARQLNIPRKEAQKYMDLYFERYPGVLEYM |
| | ERTRAQAKEQGYVETLDGRRLYLPDIKSSNGARRAAAERAAINAPMQGTAADIIKRAMIAVDAWLQAE |
| | QPRVRMIMQVHDELVFEVHKDDVDAVAKQIHQLMENCTRLDVPLLVEVGSGENWDQAHSGGSKRTADG |
| | SEFESPKKKRKV (SEQ ID NO: 191) |
| pCMV- | MAEAGITGTWYNQSGSTFTVTAGADGNLTGQYENRAQGTGCQNSPYTLTGRYNGTKLEWRVEWNNSTE |
| T7-mSA- | NCHSRTEWRGQYQGGAEARINTQWNLTYEGGSGPATEQGQDTFTKVKPSAASGSSAGGGGSGGGGSGG |
| nCas9 | GGSGPKKKRKVAAAGSDKKYSIGLDIGTNSVGWAVITDEYKVPSKKFKVLGNTDRHSIKKNLIGALLF |
| (H840A)- | DSGETAEATRLKRTARRRYTRRKNRICYLQEIFSNEMAKVDDSFFHRLEESFLVEEDKKHERHPIFGN |
| ePhi29 | IVDEVAYHEKYPTIYHLRKKLVDSTDKADLRLIYLALAHMIKFRGHFLIEGDLNPDNSDVDKLFIQLV |
| (D169A, | QTYNQLFEENPINASGVDAKAILSARLSKSRRLENLIAQLPGEKKNGLFGNLIALSLGLTPNFKSNFD |
| M8R, V51A, | LAEDAKLQLSKDTYDDDLDNLLAQIGDQYADLFLAAKNLSDAILLSDILRVNTEITKAPLSASMIKRY |
| M97T, G197D, | DEHHQDLTLLKALVRQQLPEKYKEIFFDQSKNGYAGYIDGGASQEEFYKFIKPILEKMDGTEELLVKL |
| E221K, | NREDLLRKQRTFDNGSIPHQIHLGELHAILRRQEDFYPFLKDNREKIEKILTFRIPYYVGPLARGNSR |
| Q497P, | FAWMTRKSEETITPWNFEEVVDKGASAQSFIERMTNFDKNLPNEKVLPKHSLLYEYFTVYNELTKVKY |
| K512E, | VTEGMRKPAFLSGEQKKAIVDLLFKTNRKVTVKQLKEDYFKKIECFDSVEISGVEDRFNASLGTYHDL |
| F526L) | LKIIKDKDFLDNEENEDILEDIVLTLTLFEDREMIEERLKTYAHLFDDKVMKQLKRRRYTGWGRLSRK |
| | LINGIRDKQSGKTILDFLKSDGFANRNFMQLIHDDSLTFKEDIQKAQVSGQGDSLHEHIANLAGSPAI |
| | KKGILQTVKVVDELVKVMGRHKPENIVIEMARENQTTQKGQKNSRERMKRIEEGIKELGSQILKEHPV |
| | ENTQLQNEKLYLYYLQNGRDMYVDQELDINRLSDYDVDAIVPQSFLKDDSIDNKVLTRSDKNRGKSDN |
| | VPSEEVVKKMKNYWRQLLNAKLITQRKFDNLTKAERGGLSELDKAGFIKRQLVETRQITKHVAQILDS |
| | RMNTKYDENDKLIREVKVITLKSKLVSDFRKDFQFYKVREINNYHHAHDAYLNAVVGTALIKKYPKLE |
| | SEFVYGDYKVYDVRKMIAKSEQEIGKATAKYFFYSNIMNFFKTEITLANGEIRKRPLIETNGETGEIV |
| | WDKGRDFATVRKVLSMPQVNIVKKTEVQTGGFSKESILPKRNSDKLIARKKDWDPKKYGGFDSPTVAY |
| | SVLVVAKVEKGKSKKLKSVKELLGITIMERSSFEKNPIDFLEAKGYKEVKKDLIIKLPKYSLFELENG |
| | RKRMLASAGELQKGNELALPSKYVNFLYLASHYEKLKGSPEDNEQKQLFVEQHKHYLDEIIEQISEFS |
| | KRVILADANLDKVLSAYNKHRDKPIREQAENIIHLFTLTNLGAPAAFKYFDTTIDRKRYTSTKEVLDA |
| | TLIHQSITGLYETRIDLSQLGGDSGGSSGGSKRTADGSEFESPKKKRKVSGGSSGGSKHMPRKRYSCD |
| | FETTTKVEDCRVWAYGYMNIEDHSEYKIGNSLDEFMAWALKVQADLYFHNLKFDGAFIINWLERNGFK |
| | WSADGLPNTYNTIISRTGQWYMIDICLGYKGKRKIHTVIYDSLKKLPFPVKKIAKDFKLTVLKGDIDY |
| | HKERPVGYKITPEEYAYIKNAIQIIAEALLIQFKQGLDRMTAGSDSLKDFKDIITTKKFKKVFPTLSL |
| | GLDKKVRYAYRGGFTWLNDRFKEKEIGEGMVFDVNSLYPAQMYSRLLPYGEPIVFEGKYVWDEDYPLH |
| | IQHIRCEFELKEGYIPTIQIKRSRFYKGNEYLKSSGGEIADLWLSNVDLELMKEHYDLYNVEYISGLK |
| | FKATTGLFKDFIDKWTYIKTTSEGAIKQLAKLMLNSLYGKFASNPDVTGKVPYLKENGALGFRLGEEE |
| | TKDPVYTPMGVFITAWARYTTITAAQACYDRIIYCDTDSIHLTGTEIPDVIKDIVDPKKLGYWAHEST |
| | FKRAKYLRPKTYIQDIYMKEVDGELVEGSPDDYTDIKLSVKCAGMTDKIKKEVTFENFKVGFSRKMKP |
| | KPVQVPGGVVLVDDTFTIKSGGSKRTADGSEFESPKKKRKV (SEQ ID NO: 192) |
| pCMV- | MSPSKKNGRSGPQPHKRWVFTLNNPSEDERKKIRDLPISLFDYFIVGEEGNEEGRTPHLQGFANFVKK |
| T7- | QTFNKVKWYLGARCHIEKAKGTDQQNKEYCSKEGNLLMECGAPRSQGQRSGGSSGGSSGSETPGTSES |
| PCV2- | ATPESSGGSSGGSSVISYDNYVTILDEETLKAWIAKLEKAPVFAFATATDSLDNISANLVGLSFAIEP |
| Ecklenow | GVAAYIPVAHDYLDAPDQISRERALELLKPLLEDEKALKVGQNLKYDRGILANYGIELRGIAFDTMLE |
| (-exo; D355A/ | SYILNSVAGRHDMDSLAERWLKHKTITFEEIAGKGKNQLTFNQIALEEAGRYAAEDADVTLQLHLKMW |
| E357A) | PDLQKHKGPLNVFENIEMPLVPVLSRIERNGVKIDPKVLHNHSEELTLRLAELEKKAHEIAGEEFNLS |
| | STKQLQTILFEKQGIKPLKKTPGGAPSTSEEVLEELALDYPLPKVILEYRGLAKLKSTYTDKLPLMIN |
| | PKTGRVHTSYHQAVTATGRLSSTDPNLQNIPVRNEEGRRIRQAFIAPEDYVIVSADYSQIELRIMAHL |
| | SRDKGLLTAFAEGKDIHRATAAEVFGLPLETVTSEQRRSAKAINFGLIYGMSAFGLARQLNIPRKEAQ |
| | KYMDLYFERYPGVLEYMERTRAQAKEQGYVETLDGRRLYLPDIKSSNGARRAAAERAAINAPMQGTAA |
| | DIIKRAMIAVDAWLQAEQPRVRMIMQVHDELVFEVHKDDVDAVAKQIHQLMENCTRLDVPLLVEVGSG |
| | ENWDQAHSGGSSGGSKRTADGSEFESPKKKRKV (SEQ ID NO: 193) |
| pCMV- | MSPSKKNGRSGPQPHKRWVFTLNNPSEDERKKIRDLPISLFDYFIVGEEGNEEGRTPHLQGFANFVKK |
| T7- | QTFNKVKWYLGARCHIEKAKGTDQQNKEYCSKEGNLLMECGAPRSQGQRSGGSSGGSSGSETPGTSES |
| PCV2- | ATPESSGGSSGGSSKHMPRKRYSCDFETTTKVEDCRVWAYGYMNIEDHSEYKIGNSLDEFMAWALKVQ |
| ePhi29 | ADLYFHNLKFDGAFIINWLERNGFKWSADGLPNTYNTIISRTGQWYMIDICLGYKGKRKIHTVIYDSL |
| (D169A, | KKLPFPVKKIAKDFKLTVLKGDIDYHKERPVGYKITPEEYAYIKNAIQIIAEALLIQFKQGLDRMTAG |
| M8R, V51A, | SDSLKDFKDIITTKKFKKVFPTLSLGLDKKVRYAYRGGFTWLNDRFKEKEIGEGMVFDVNSLYPAQMY |
| M97T, | SRLLPYGEPIVFEGKYVWDEDYPLHIQHIRCEFELKEGYIPTIQIKRSRFYKGNEYLKSSGGEIADLW |
| G197D, | LSNVDLELMKEHYDLYNVEYISGLKFKATTGLFKDFIDKWTYIKTTSEGAIKQLAKLMLNSLYGKFAS |
| E221K, | NPDVTGKVPYLKENGALGFRLGEEETKDPVYTPMGVFITAWARYTTITAAQACYDRIIYCDTDSIHLT |
| Q497P, | GTEIPDVIKDIVDPKKLGYWAHESTFKRAKYLRPKTYIQDIYMKEVDGELVEGSPDDYTDIKLSVKCA |
| K512E, | GMTDKIKKEVTFENFKVGFSRKMKPKPVQVPGGVVLVDDTFTIKSGGSSGGSKRTADGSEFESPKKKR |
| F526L) | KV (SEQ ID NO: 194) |
| pCMV- | MGAQPARSMAEAGITGTWYNQSGSTFTVTAGADGNLTGQYENRAQGTGCQNSPYTLTGRYNGTKLEWR |
| T7-mSA- | VEWNNSTENCHSRTEWRGQYQGGAEARINTQWNLTYEGGSGPATEQGQDTFTKVKPSAASGSSAGGGG |
| Ecklenow | SGGGGSGGGGSGPKKKRKVAAAGSVISYDNYVTILDEETLKAWIAKLEKAPVFAFATATDSLDNISAN |
| (-exo; D355A/ | LVGLSFAIEPGVAAYIPVAHDYLDAPDQISRERALELLKPLLEDEKALKVGQNLKYDRGILANYGIEL |
| E357A) | RGIAFDTMLESYILNSVAGRHDMDSLAERWLKHKTITFEEIAGKGKNQLTFNQIALEEAGRYAAEDAD |
| | VTLQLHLKMWPDLQKHKGPLNVFENIEMPLVPVLSRIERNGVKIDPKVLHNHSEELTLRLAELEKKAH |
| | EIAGEEFNLSSTKQLQTILFEKQGIKPLKKTPGGAPSTSEEVLEELALDYPLPKVILEYRGLAKLKST |
| | YTDKLPLMINPKTGRVHTSYHQAVTATGRLSSTDPNLQNIPVRNEEGRRIRQAFIAPEDYVIVSADYS |
| | QIELRIMAHLSRDKGLLTAFAEGKDIHRATAAEVFGLPLETVTSEQRRSAKAINFGLIYGMSAFGLAR |
| | QLNIPRKEAQKYMDLYFERYPGVLEYMERTRAQAKEQGYVETLDGRRLYLPDIKSSNGARRAAAERAA |
| | INAPMQGTAADIIKRAMIAVDAWLQAEQPRVRMIMQVHDELVFEVHKDDVDAVAKQIHQLMENCTRLD |
| | VPLLVEVGSGENWDQAHSGGSSGGSKRTADGSEFESPKKKRKV (SEQ ID NO: 195) |
| pCMV- | MGAQPARSMAEAGITGTWYNQSGSTFTVTAGADGNLTGQYENRAQGTGCQNSPYTLTGRYNGTKLEWR |
| T7-mSA- | VEWNNSTENCHSRTEWRGQYQGGAEARINTQWNLTYEGGSGPATEQGQDTFTKVKPSAASGSSAGGGG |
| ePhi29 | SGGGGSGGGGSGPKKKRKVAAAGSKHMPRKRYSCDFETTTKVEDCRVWAYGYMNIEDHSEYKIGNSLD |
| D169A, | EFMAWALKVQADLYFHNLKFDGAFIINWLERNGFKWSADGLPNTYNTIISRTGQWYMIDICLGYKGKR |
| (M8R, V51A, | KIHTVIYDSLKKLPFPVKKIAKDFKLTVLKGDIDYHKERPVGYKITPEEYAYIKNAIQIIAEALLIQF |
| M97T, | KQGLDRMTAGSDSLKDFKDIITTKKFKKVFPTLSLGLDKKVRYAYRGGFTWLNDRFKEKEIGEGMVFD |
| G197D, | VNSLYPAQMYSRLLPYGEPIVFEGKYVWDEDYPLHIQHIRCEFELKEGYIPTIQIKRSRFYKGNEYLK |
| E221K, | SSGGEIADLWLSNVDLELMKEHYDLYNVEYISGLKFKATTGLFKDFIDKWTYIKTTSEGAIKQLAKLM |
| Q497P, | LNSLYGKFASNPDVTGKVPYLKENGALGFRLGEEETKDPVYTPMGVFITAWARYTTITAAQACYDRII |
| K512E, | YCDTDSIHLTGTEIPDVIKDIVDPKKLGYWAHESTFKRAKYLRPKTYIQDIYMKEVDGELVEGSPDDY |
| | TDIKLSVKCAGMTDKIKKEVTFENFKVGFSRKMKPKPVQVPGGVVLVDDTFTIKSGGSSGGSKRTADG |
| | SEFESPKKKRKV (SEQ ID NO: 196) |
| F526L) | MSPSKKNGRSGPQPHKRWVFTLNNPSEDERKKIRDLPISLFDYFIVGEEGNEEGRTPHLQGFANFVKKQ |
| T7- | TFNKVKWYLGARCHIEKAKGTDQQNKEYCSKEGNLLMECGAPRSQGQRSGGSSGSETPGTSESATPESS |
| 5′UTR- | GGSDKKYSIGLDIGTNSVGWAVITDEYKVPSKKFKVLGNTDRHSIKKNLIGALLFDSGETAEATRLKRTARR |
| PCV2- | RYTRRKNRICYLQEIFSNEMAKVDDSFFHRLEESFLVEEDKKHERHPIFGNIVDEVAYHEKYPTIYHLRKKL |
| nCas9 | VDSTDKADLRLIYLALAHMIKFRGHFLIEGDLNPDNSDVDKLFIQLVQTYNQLFEENPINASGVDAKAILSA |
| (H840A)- | RLSKSRRLENLIAQLPGEKKNGLFGNLIALSLGLTPNFKSNFDLAEDAKLQLSKDTYDDDLDNLLAQIGDQY |
| Ecklenow | ADLFLAAKNLSDAILLSDILRVNTEITKAPLSASMIKRYDEHHQDLTLLKALVRQQLPEKYKEIFFDQSKNGY |
| (exo-)- | AGYIDGGASQEEFYKFIKPILEKMDGTEELLVKLNREDLLRKQRTFDNGSIPHQIHLGELHAILRRQEDFYP |
| 3′UTR- | FLKDNREKIEKILTFRIPYYVGPLARGNSRFAWMTRKSEETITPWNFEEVVDKGASAQSFIERMTNFDKNL |
| polyA | PNEKVLPKHSLLYEYFTVYNELTKVKYVTEGMRKPAFLSGEQKKAIVDLLFKTNRKVTVKQLKEDYFKKIEC |
| | FDSVEISGVEDRFNASLGTYHDLLKIIKDKDFLDNEENEDILEDIVLTLTLFEDREMIEERLKTYAHLFDDKV |
| | MKQLKRRRYTGWGRLSRKLINGIRDKQSGKTILDFLKSDGFANRNFMQLIHDDSLTFKEDIQKAQVSGQ |
| | GDSLHEHIANLAGSPAIKKGILQTVKVVDELVKVMGRHKPENIVIEMARENQTTQKGQKNSRERMKRIE |
| | EGIKELGSQILKEHPVENTQLQNEKLYLYYLQNGRDMYVDQELDINRLSDYDVDAIVPQSFLKDDSIDNKV |
| | LTRSDKNRGKSDNVPSEEVVKKMKNYWRQLLNAKLITQRKFDNLTKAERGGLSELDKAGFIKRQLVETR |
| | QITKHVAQILDSRMNTKYDENDKLIREVKVITLKSKLVSDFRKDFQFYKVREINNYHHAHDAYLNAVVGT |
| | ALIKKYPKLESEFVYGDYKVYDVRKMIAKSEQEIGKATAKYFFYSNIMNFFKTEITLANGEIRKRPLIETNGE |
| | TGEIVWDKGRDFATVRKVLSMPQVNIVKKTEVQTGGFSKESILPKRNSDKLIARKKDWDPKKYGGFDSP |
| | TVAYSVLVVAKVEKGKSKKLKSVKELLGITIMERSSFEKNPIDFLEAKGYKEVKKDLIIKLPKYSLFELENGRK |
| | RMLASAGELQKGNELALPSKYVNFLYLASHYEKLKGSPEDNEQKQLFVEQHKHYLDEIIEQISEFSKRVILA |
| | DANLDKVLSAYNKHRDKPIREQAENIIHLFTLTNLGAPAAFKYFDTTIDRKRYTSTKEVLDATLIHQSITGLY |
| | ETRIDLSQLGGDSGGSSGGSKRTADGSEFESPKKKRKVSGGSSGGSVISYDNYVTILDEETLKAWIAKLEKA |
| | PVFAFATATDSLDNISANLVGLSFAIEPGVAAYIPVAHDYLDAPDQISRERALELLKPLLEDEKALKVGQNL |
| | KYDRGILANYGIELRGIAFDTMLESYILNSVAGRHDMDSLAERWLKHKTITFEEIAGKGKNQLTFNQIALE |
| | EAGRYAAEDADVTLQLHLKMWPDLQKHKGPLNVFENIEMPLVPVLSRIERNGVKIDPKVLHNHSEELTL |
| | RLAELEKKAHEIAGEEFNLSSTKQLQTILFEKQGIKPLKKTPGGAPSTSEEVLEELALDYPLPKVILEYRGLAK |
| | LKSTYTDKLPLMINPKTGRVHTSYHQAVTATGRLSSTDPNLQNIPVRNEEGRRIRQAFIAPEDYVIVSADY |
| | SQIELRIMAHLSRDKGLLTAFAEGKDIHRATAAEVFGLPLETVTSEQRRSAKAINFGLIYGMSAFGLARQL |
| | NIPRKEAQKYMDLYFERYPGVLEYMERTRAQAKEQGYVETLDGRRLYLPDIKSSNGARRAAAERAAINA |
| | PMQGTAADIIKRAMIAVDAWLQAEQPRVRMIMQVHDELVFEVHKDDVDAVAKQIHQLMENCTRLDV |
| | PLLVEVGSGENWDQAHSGGSKRTADGSEFESPKKKRKV (SEQ ID NO: 131) |
| pCMV- | MASNFTQFVLVDNGGTGDVTVAPSNFANGVAEWISSNSRSQAYKVTCSVRQSSAQKRKYTIKVEVPKV |
| T7- | ATQTVGGVELPVAAWRSYLNMELTIPIFATNSDCELIVKAMQGLLKDGNPIPSAIAANSGIYSGGSSG |
| MCP | GSSGSETPGTSESATPESSGGSSGGSSDKKYSIGLDIGTNSVGWAVITDEYKVPSKKFKVLGNTDRHS |
| (N55K)- | IKKNLIGALLFDSGETAEATRLKRTARRRYTRRKNRICYLQEIFSNEMAKVDDSFFHRLEESFLVEED |
| nCas9- | KKHERHPIFGNIVDEVAYHEKYPTIYHLRKKLVDSTDKADLRLIYLALAHMIKFRGHFLIEGDLNPDN |
| Ecklenow | SDVDKLFIQLVQTYNQLFEENPINASGVDAKAILSARLSKSRRLENLIAQLPGEKKNGLFGNLIALSL |
| | GLTPNFKSNFDLAEDAKLQLSKDTYDDDLDNLLAQIGDQYADLFLAAKNLSDAILLSDILRVNTEITK |
| | APLSASMIKRYDEHHQDLTLLKALVRQQLPEKYKEIFFDQSKNGYAGYIDGGASQEEFYKFIKPILEK |
| | MDGTEELLVKLNREDLLRKQRTFDNGSIPHQIHLGELHAILRRQEDFYPFLKDNREKIEKILTFRIPY |
| | YVGPLARGNSRFAWMTRKSEETITPWNFEEVVDKGASAQSFIERMTNFDKNLPNEKVLPKHSLLYEYF |
| | TVYNELTKVKYVTEGMRKPAFLSGEQKKAIVDLLFKTNRKVTVKQLKEDYFKKIECFDSVEISGVEDR |
| | FNASLGTYHDLLKIIKDKDFLDNEENEDILEDIVLTLTLFEDREMIEERLKTYAHLFDDKVMKQLKRR |
| | RYTGWGRLSRKLINGIRDKQSGKTILDFLKSDGFANRNFMQLIHDDSLTFKEDIQKAQVSGQGDSLHE |
| | HIANLAGSPAIKKGILQTVKVVDELVKVMGRHKPENIVIEMARENQTTQKGQKNSRERMKRIEEGIKE |
| | LGSQILKEHPVENTQLQNEKLYLYYLQNGRDMYVDQELDINRLSDYDVDAIVPQSFLKDDSIDNKVLT |
| | RSDKNRGKSDNVPSEEVVKKMKNYWRQLLNAKLITQRKFDNLTKAERGGLSELDKAGFIKRQLVETRQ |
| | ITKHVAQILDSRMNTKYDENDKLIREVKVITLKSKLVSDFRKDFQFYKVREINNYHHAHDAYLNAVVG |
| | TALIKKYPKLESEFVYGDYKVYDVRKMIAKSEQEIGKATAKYFFYSNIMNFFKTEITLANGEIRKRPL |
| | IETNGETGEIVWDKGRDFATVRKVLSMPQVNIVKKTEVQTGGFSKESILPKRNSDKLIARKKDWDPKK |
| | YGGFDSPTVAYSVLVVAKVEKGKSKKLKSVKELLGITIMERSSFEKNPIDFLEAKGYKEVKKDLIIKL |
| | PKYSLFELENGRKRMLASAGELQKGNELALPSKYVNFLYLASHYEKLKGSPEDNEQKQLFVEQHKHYL |
| | DEIIEQISEFSKRVILADANLDKVLSAYNKHRDKPIREQAENIIHLFTLTNLGAPAAFKYFDTTIDRK |
| | RYTSTKEVLDATLIHQSITGLYETRIDLSQLGGDSGGSSGGSKRTADGSEFESPKKKRKVSGGSSGGS |
| | VISYDNYVTILDEETLKAWIAKLEKAPVFAFATATDSLDNISANLVGLSFAIEPGVAAYIPVAHDYLD |
| | APDQISRERALELLKPLLEDEKALKVGQNLKYDRGILANYGIELRGIAFDTMLESYILNSVAGRHDMD |
| | SLAERWLKHKTITFEEIAGKGKNQLTFNQIALEEAGRYAAEDADVTLQLHLKMWPDLQKHKGPLNVFE |
| | NIEMPLVPVLSRIERNGVKIDPKVLHNHSEELTLRLAELEKKAHEIAGEEFNLSSTKQLQTILFEKQG |
| | IKPLKKTPGGAPSTSEEVLEELALDYPLPKVILEYRGLAKLKSTYTDKLPLMINPKTGRVHTSYHQAV |
| | TATGRLSSTDPNLQNIPVRNEEGRRIRQAFIAPEDYVIVSADYSQIELRIMAHLSRDKGLLTAFAEGK |
| | DIHRATAAEVFGLPLETVTSEQRRSAKAINFGLIYGMSAFGLARQLNIPRKEAQKYMDLYFERYPGVL |
| | EYMERTRAQAKEQGYVETLDGRRLYLPDIKSSNGARRAAAERAAINAPMQGTAADIIKRAMIAVDAWL |
| | QAEQPRVRMIMQVHDELVFEVHKDDVDAVAKQIHQLMENCTRLDVPLLVEVGSGENWDQAHSGGSKRT |
| | ADGSEFESPKKKRKV (SEQ ID NO: 198) |
| pCMV- | MASNFTQFVLVDNGGTGDVTVAPSNFANGVAEWISSNSRSQAYKVTCSVRQSSAQKRKYTIKVEVPKV |
| T7- | ATQTVGGVELPVAAWRSYLNMELTIPIFATNSDCELIVKAMQGLLKDGNPIPSAIAANSGIYSGGSSG |
| MCP | GSSGSETPGTSESATPESSGGSSGGSSDKKYSIGLDIGTNSVGWAVITDEYKVPSKKFKVLGNTDRHS |
| (N55K)- | IKKNLIGALLFDSGETAEATRLKRTARRRYTRRKNRICYLQEIFSNEMAKVDDSFFHRLEESFLVEED |
| nCas9- | KKHERHPIFGNIVDEVAYHEKYPTIYHLRKKLVDSTDKADLRLIYLALAHMIKFRGHFLIEGDLNPDN |
| ePhi29 | SDVDKLFIQLVQTYNQLFEENPINASGVDAKAILSARLSKSRRLENLIAQLPGEKKNGLFGNLIALSL |
| (D169A) | GLTPNFKSNFDLAEDAKLQLSKDTYDDDLDNLLAQIGDQYADLFLAAKNLSDAILLSDILRVNTEITK |
| | APLSASMIKRYDEHHQDLTLLKALVRQQLPEKYKEIFFDQSKNGYAGYIDGGASQEEFYKFIKPILEK |
| | MDGTEELLVKLNREDLLRKQRTFDNGSIPHQIHLGELHAILRRQEDFYPFLKDNREKIEKILTFRIPY |
| | YVGPLARGNSRFAWMTRKSEETITPWNFEEVVDKGASAQSFIERMTNFDKNLPNEKVLPKHSLLYEYF |
| | TVYNELTKVKYVTEGMRKPAFLSGEQKKAIVDLLFKTNRKVTVKQLKEDYFKKIECFDSVEISGVEDR |
| | FNASLGTYHDLLKIIKDKDFLDNEENEDILEDIVLTLTLFEDREMIEERLKTYAHLFDDKVMKQLKRR |
| | RYTGWGRLSRKLINGIRDKQSGKTILDFLKSDGFANRNFMQLIHDDSLTFKEDIQKAQVSGQGDSLHE |
| | HIANLAGSPAIKKGILQTVKVVDELVKVMGRHKPENIVIEMARENQTTQKGQKNSRERMKRIEEGIKE |
| | LGSQILKEHPVENTQLQNEKLYLYYLQNGRDMYVDQELDINRLSDYDVDAIVPQSFLKDDSIDNKVLT |
| | RSDKNRGKSDNVPSEEVVKKMKNYWRQLLNAKLITQRKFDNLTKAERGGLSELDKAGFIKRQLVETRQ |
| | ITKHVAQILDSRMNTKYDENDKLIREVKVITLKSKLVSDFRKDFQFYKVREINNYHHAHDAYLNAVVG |
| | TALIKKYPKLESEFVYGDYKVYDVRKMIAKSEQEIGKATAKYFFYSNIMNFFKTEITLANGEIRKRPL |
| | IETNGETGEIVWDKGRDFATVRKVLSMPQVNIVKKTEVQTGGFSKESILPKRNSDKLIARKKDWDPKK |
| | YGGFDSPTVAYSVLVVAKVEKGKSKKLKSVKELLGITIMERSSFEKNPIDFLEAKGYKEVKKDLIIKL |
| | PKYSLFELENGRKRMLASAGELQKGNELALPSKYVNFLYLASHYEKLKGSPEDNEQKQLFVEQHKHYL |
| | DEIIEQISEFSKRVILADANLDKVLSAYNKHRDKPIREQAENIIHLFTLTNLGAPAAFKYFDTTIDRK |
| | RYTSTKEVLDATLIHQSITGLYETRIDLSQLGGDSGGSSGGSKRTADGSEFESPKKKRKVSGGSSGGS |
| | KHMPRKRYSCDFETTTKVEDCRVWAYGYMNIEDHSEYKIGNSLDEFMAWALKVQADLYFHNLKFDGAF |
| | IINWLERNGFKWSADGLPNTYNTIISRTGQWYMIDICLGYKGKRKIHTVIYDSLKKLPFPVKKIAKDF |
| | KLTVLKGDIDYHKERPVGYKITPEEYAYIKNAIQIIAEALLIQFKQGLDRMTAGSDSLKDFKDIITTK |
| | KFKKVFPTLSLGLDKKVRYAYRGGFTWLNDRFKEKEIGEGMVFDVNSLYPAQMYSRLLPYGEPIVFEG |
| | KYVWDEDYPLHIQHIRCEFELKEGYIPTIQIKRSRFYKGNEYLKSSGGEIADLWLSNVDLELMKEHYD |
| | LYNVEYISGLKFKATTGLFKDFIDKWTYIKTTSEGAIKQLAKLMLNSLYGKFASNPDVTGKVPYLKEN |
| | GALGFRLGEEETKDPVYTPMGVFITAWARYTTITAAQACYDRIIYCDTDSIHLTGTEIPDVIKDIVDP |
| | KKLGYWAHESTFKRAKYLRPKTYIQDIYMKEVDGELVEGSPDDYTDIKLSVKCAGMTDKIKKEVTFEN |
| | FKVGFSRKMKPKPVQVPGGVVLVDDTFTIKSGGSKRTADGSEFESPKKKRKV (SEQ ID NO: 199) |
| pCMV- | MASNFTQFVLVDNGGTGDVTVAPSNFANGVAEWISSNSRSQAYKVTCSVRQSSAQKRKYTIKVEVPKV |
| T7- | ATQTVGGVELPVAAWRSYLNMELTIPIFATNSDCELIVKAMQGLLKDGNPIPSAIAANSGIYSGGSSG |
| MCP | GSSGSETPGTSESATPESSGGSSGGSSVISYDNYVTILDEETLKAWIAKLEKAPVFAFATATDSLDNI |
| (N55K)- | SANLVGLSFAIEPGVAAYIPVAHDYLDAPDQISRERALELLKPLLEDEKALKVGQNLKYDRGILANYG |
| Ecklenow | IELRGIAFDTMLESYILNSVAGRHDMDSLAERWLKHKTITFEEIAGKGKNQLTFNQIALEEAGRYAAE |
| | DADVTLQLHLKMWPDLQKHKGPLNVFENIEMPLVPVLSRIERNGVKIDPKVLHNHSEELTLRLAELEK |
| | KAHEIAGEEFNLSSTKQLQTILFEKQGIKPLKKTPGGAPSTSEEVLEELALDYPLPKVILEYRGLAKL |
| | KSTYTDKLPLMINPKTGRVHTSYHQAVTATGRLSSTDPNLQNIPVRNEEGRRIRQAFIAPEDYVIVSA |
| | DYSQIELRIMAHLSRDKGLLTAFAEGKDIHRATAAEVFGLPLETVTSEQRRSAKAINFGLIYGMSAFG |
| | LARQLNIPRKEAQKYMDLYFERYPGVLEYMERTRAQAKEQGYVETLDGRRLYLPDIKSSNGARRAAAE |
| | RAAINAPMQGTAADIIKRAMIAVDAWLQAEQPRVRMIMQVHDELVFEVHKDDVDAVAKQIHQLMENCT |
| | RLDVPLLVEVGSGENWDQAHSGGSSGGSKRTADGSEFESPKKKRKV (SEQ ID NO: 200) |
| pCMV- | MASNFTQFVLVDNGGTGDVTVAPSNFANGVAEWISSNSRSQAYKVTCSVRQSSAQKRKYTIKVEVPKV |
| T7- | ATQTVGGVELPVAAWRSYLNMELTIPIFATNSDCELIVKAMQGLLKDGNPIPSAIAANSGIYSGGSSG |
| MCP | GSSGSETPGTSESATPESSGGSSGGSSKHMPRKRYSCDFETTTKVEDCRVWAYGYMNIEDHSEYKIGN |
| (N55K)- | SLDEFMAWALKVQADLYFHNLKFDGAFIINWLERNGFKWSADGLPNTYNTIISRTGQWYMIDICLGYK |
| ePhi28 | GKRKIHTVIYDSLKKLPFPVKKIAKDFKLTVLKGDIDYHKERPVGYKITPEEYAYIKNAIQIIAEALL |
| (D169A) | IQFKQGLDRMTAGSDSLKDFKDIITTKKFKKVFPTLSLGLDKKVRYAYRGGFTWLNDRFKEKEIGEGM |
| | VFDVNSLYPAQMYSRLLPYGEPIVFEGKYVWDEDYPLHIQHIRCEFELKEGYIPTIQIKRSRFYKGNE |
| | YLKSSGGEIADLWLSNVDLELMKEHYDLYNVEYISGLKFKATTGLFKDFIDKWTYIKTTSEGAIKQLA |
| | KLMLNSLYGKFASNPDVTGKVPYLKENGALGFRLGEEETKDPVYTPMGVFITAWARYTTITAAQACYD |
| | RIIYCDTDSIHLTGTEIPDVIKDIVDPKKLGYWAHESTFKRAKYLRPKTYIQDIYMKEVDGELVEGSP |
| | DDYTDIKLSVKCAGMTDKIKKEVTFENFKVGFSRKMKPKPVQVPGGVVLVDDTFTIKSGGSSGGSKRT |
| | ADGSEFESPKKKRKV (SEQ ID NO: 201) |
| pCMV- | MASNFTQFVLVDNGGTGDVTVAPSNFANGVAEWISSNSRSQAYKVTCSVRQSSAQNRKYTIKVEVPKV |
| T7-MCP- | ATQTVGGVELPVAAWRSYLNMELTIPIFATNSDCELIVKAMQGLLKDGNPIPSAIAANSGIYSGGSSG |
| nCas9- | GSSGSETPGTSESATPESSGGSSGGSSDKKYSIGLDIGTNSVGWAVITDEYKVPSKKFKVLGNTDRHS |
| Ecklenow | IKKNLIGALLFDSGETAEATRLKRTARRRYTRRKNRICYLQEIFSNEMAKVDDSFFHRLEESFLVEED |
| | KKHERHPIFGNIVDEVAYHEKYPTIYHLRKKLVDSTDKADLRLIYLALAHMIKFRGHFLIEGDLNPDN |
| | SDVDKLFIQLVQTYNQLFEENPINASGVDAKAILSARLSKSRRLENLIAQLPGEKKNGLFGNLIALSL |
| | GLTPNFKSNFDLAEDAKLQLSKDTYDDDLDNLLAQIGDQYADLFLAAKNLSDAILLSDILRVNTEITK |
| | APLSASMIKRYDEHHQDLTLLKALVRQQLPEKYKEIFFDQSKNGYAGYIDGGASQEEFYKFIKPILEK |
| | MDGTEELLVKLNREDLLRKQRTFDNGSIPHQIHLGELHAILRRQEDFYPFLKDNREKIEKILTFRIPY |
| | YVGPLARGNSRFAWMTRKSEETITPWNFEEVVDKGASAQSFIERMTNFDKNLPNEKVLPKHSLLYEYF |
| | TVYNELTKVKYVTEGMRKPAFLSGEQKKAIVDLLFKTNRKVTVKQLKEDYFKKIECFDSVEISGVEDR |
| | FNASLGTYHDLLKIIKDKDFLDNEENEDILEDIVLTLTLFEDREMIEERLKTYAHLFDDKVMKQLKRR |
| | RYTGWGRLSRKLINGIRDKQSGKTILDFLKSDGFANRNFMQLIHDDSLTFKEDIQKAQVSGQGDSLHE |
| | HIANLAGSPAIKKGILQTVKVVDELVKVMGRHKPENIVIEMARENQTTQKGQKNSRERMKRIEEGIKE |
| | LGSQILKEHPVENTQLQNEKLYLYYLQNGRDMYVDQELDINRLSDYDVDAIVPQSFLKDDSIDNKVLT |
| | RSDKNRGKSDNVPSEEVVKKMKNYWRQLLNAKLITQRKFDNLTKAERGGLSELDKAGFIKRQLVETRQ |
| | ITKHVAQILDSRMNTKYDENDKLIREVKVITLKSKLVSDFRKDFQFYKVREINNYHHAHDAYLNAVVG |
| | TALIKKYPKLESEFVYGDYKVYDVRKMIAKSEQEIGKATAKYFFYSNIMNFFKTEITLANGEIRKRPL |
| | IETNGETGEIVWDKGRDFATVRKVLSMPQVNIVKKTEVQTGGFSKESILPKRNSDKLIARKKDWDPKK |
| | YGGFDSPTVAYSVLVVAKVEKGKSKKLKSVKELLGITIMERSSFEKNPIDFLEAKGYKEVKKDLIIKL |
| | PKYSLFELENGRKRMLASAGELQKGNELALPSKYVNFLYLASHYEKLKGSPEDNEQKQLFVEQHKHYL |
| | DEIIEQISEFSKRVILADANLDKVLSAYNKHRDKPIREQAENIIHLFTLTNLGAPAAFKYFDTTIDRK |
| | RYTSTKEVLDATLIHQSITGLYETRIDLSQLGGDSGGSSGGSKRTADGSEFESPKKKRKVSGGSSGGS |
| | VISYDNYVTILDEETLKAWIAKLEKAPVFAFATATDSLDNISANLVGLSFAIEPGVAAYIPVAHDYLD |
| | APDQISRERALELLKPLLEDEKALKVGQNLKYDRGILANYGIELRGIAFDTMLESYILNSVAGRHDMD |
| | SLAERWLKHKTITFEEIAGKGKNQLTFNQIALEEAGRYAAEDADVTLQLHLKMWPDLQKHKGPLNVFE |
| | NIEMPLVPVLSRIERNGVKIDPKVLHNHSEELTLRLAELEKKAHEIAGEEFNLSSTKQLQTILFEKQG |
| | IKPLKKTPGGAPSTSEEVLEELALDYPLPKVILEYRGLAKLKSTYTDKLPLMINPKTGRVHTSYHQAV |
| | TATGRLSSTDPNLQNIPVRNEEGRRIRQAFIAPEDYVIVSADYSQIELRIMAHLSRDKGLLTAFAEGK |
| | DIHRATAAEVFGLPLETVTSEQRRSAKAINFGLIYGMSAFGLARQLNIPRKEAQKYMDLYFERYPGVL |
| | EYMERTRAQAKEQGYVETLDGRRLYLPDIKSSNGARRAAAERAAINAPMQGTAADIIKRAMIAVDAWL |
| | QAEQPRVRMIMQVHDELVFEVHKDDVDAVAKQIHQLMENCTRLDVPLLVEVGSGENWDQAHSGGSKRT |
| | ADGSEFESPKKKRKV (SEQ ID NO: 202) |
| pCMV- | MASNFTQFVLVDNGGTGDVTVAPSNFANGVAEWISSNSRSQAYKVTCSVRQSSAQNRKYTIKVEVPKV |
| T7-MCP- | ATQTVGGVELPVAAWRSYLNMELTIPIFATNSDCELIVKAMQGLLKDGNPIPSAIAANSGIYSGGSSG |
| nCas9- | GSSGSETPGTSESATPESSGGSSGGSSDKKYSIGLDIGTNSVGWAVITDEYKVPSKKFKVLGNTDRHS |
| ePhi29 | IKKNLIGALLFDSGETAEATRLKRTARRRYTRRKNRICYLQEIFSNEMAKVDDSFFHRLEESFLVEED |
| (D169A) | KKHERHPIFGNIVDEVAYHEKYPTIYHLRKKLVDSTDKADLRLIYLALAHMIKFRGHFLIEGDLNPDN |
| | SDVDKLFIQLVQTYNQLFEENPINASGVDAKAILSARLSKSRRLENLIAQLPGEKKNGLFGNLIALSL |
| | GLTPNFKSNFDLAEDAKLQLSKDTYDDDLDNLLAQIGDQYADLFLAAKNLSDAILLSDILRVNTEITK |
| | APLSASMIKRYDEHHQDLTLLKALVRQQLPEKYKEIFFDQSKNGYAGYIDGGASQEEFYKFIKPILEK |
| | MDGTEELLVKLNREDLLRKQRTFDNGSIPHQIHLGELHAILRRQEDFYPFLKDNREKIEKILTFRIPY |
| | YVGPLARGNSRFAWMTRKSEETITPWNFEEVVDKGASAQSFIERMTNFDKNLPNEKVLPKHSLLYEYF |
| | TVYNELTKVKYVTEGMRKPAFLSGEQKKAIVDLLFKTNRKVTVKQLKEDYFKKIECFDSVEISGVEDR |
| | FNASLGTYHDLLKIIKDKDFLDNEENEDILEDIVLTLTLFEDREMIEERLKTYAHLFDDKVMKQLKRR |
| | RYTGWGRLSRKLINGIRDKQSGKTILDFLKSDGFANRNFMQLIHDDSLTFKEDIQKAQVSGQGDSLHE |
| | HIANLAGSPAIKKGILQTVKVVDELVKVMGRHKPENIVIEMARENQTTQKGQKNSRERMKRIEEGIKE |
| | LGSQILKEHPVENTQLQNEKLYLYYLQNGRDMYVDQELDINRLSDYDVDAIVPQSFLKDDSIDNKVLT |
| | RSDKNRGKSDNVPSEEVVKKMKNYWRQLLNAKLITQRKFDNLTKAERGGLSELDKAGFIKRQLVETRQ |
| | ITKHVAQILDSRMNTKYDENDKLIREVKVITLKSKLVSDFRKDFQFYKVREINNYHHAHDAYLNAVVG |
| | TALIKKYPKLESEFVYGDYKVYDVRKMIAKSEQEIGKATAKYFFYSNIMNFFKTEITLANGEIRKRPL |
| | IETNGETGEIVWDKGRDFATVRKVLSMPQVNIVKKTEVQTGGFSKESILPKRNSDKLIARKKDWDPKK |
| | YGGFDSPTVAYSVLVVAKVEKGKSKKLKSVKELLGITIMERSSFEKNPIDFLEAKGYKEVKKDLIIKL |
| | PKYSLFELENGRKRMLASAGELQKGNELALPSKYVNFLYLASHYEKLKGSPEDNEQKQLFVEQHKHYL |
| | DEIIEQISEFSKRVILADANLDKVLSAYNKHRDKPIREQAENIIHLFTLTNLGAPAAFKYFDTTIDRK |
| | RYTSTKEVLDATLIHQSITGLYETRIDLSQLGGDSGGSSGGSKRTADGSEFESPKKKRKVSGGSSGGS |
| | KHMPRKRYSCDFETTTKVEDCRVWAYGYMNIEDHSEYKIGNSLDEFMAWALKVQADLYFHNLKFDGAF |
| | IINWLERNGFKWSADGLPNTYNTIISRTGQWYMIDICLGYKGKRKIHTVIYDSLKKLPFPVKKIAKDF |
| | KLTVLKGDIDYHKERPVGYKITPEEYAYIKNAIQIIAEALLIQFKQGLDRMTAGSDSLKDFKDIITTK |
| | KFKKVFPTLSLGLDKKVRYAYRGGFTWLNDRFKEKEIGEGMVFDVNSLYPAQMYSRLLPYGEPIVFEG |
| | KYVWDEDYPLHIQHIRCEFELKEGYIPTIQIKRSRFYKGNEYLKSSGGEIADLWLSNVDLELMKEHYD |
| | LYNVEYISGLKFKATTGLFKDFIDKWTYIKTTSEGAIKQLAKLMLNSLYGKFASNPDVTGKVPYLKEN |
| | GALGFRLGEEETKDPVYTPMGVFITAWARYTTITAAQACYDRIIYCDTDSIHLTGTEIPDVIKDIVDP |
| | KKLGYWAHESTFKRAKYLRPKTYIQDIYMKEVDGELVEGSPDDYTDIKLSVKCAGMTDKIKKEVTFEN |
| | FKVGFSRKMKPKPVQVPGGVVLVDDTFTIKSGGSKRTADGSEFESPKKKRKV (SEQ ID NO: 203) |
| pCMV- | MDKKYSIGLDIGTNSVGWAVITDEYKVPSKKFKVLGNTDRHSIKKNLIGALLFDSGETAEATRLKRTA |
| T7- | RRRYTRRKNRICYLQEIFSNEMAKVDDSFFHRLEESFLVEEDKKHERHPIFGNIVDEVAYHEKYPTIY |
| nCas9- | HLRKKLVDSTDKADLRLIYLALAHMIKFRGHFLIEGDLNPDNSDVDKLFIQLVQTYNQLFEENPINAS |
| Ecklenow- | GVDAKAILSARLSKSRRLENLIAQLPGEKKNGLFGNLIALSLGLTPNFKSNFDLAEDAKLQLSKDTYD |
| PCV2 | DDLDNLLAQIGDQYADLFLAAKNLSDAILLSDILRVNTEITKAPLSASMIKRYDEHHQDLTLLKALVR |
| | QQLPEKYKEIFFDQSKNGYAGYIDGGASQEEFYKFIKPILEKMDGTEELLVKLNREDLLRKQRTFDNG |
| | SIPHQIHLGELHAILRRQEDFYPFLKDNREKIEKILTFRIPYYVGPLARGNSRFAWMTRKSEETITPW |
| | NFEEVVDKGASAQSFIERMTNFDKNLPNEKVLPKHSLLYEYFTVYNELTKVKYVTEGMRKPAFLSGEQ |
| | KKAIVDLLFKTNRKVTVKQLKEDYFKKIECFDSVEISGVEDRFNASLGTYHDLLKIIKDKDFLDNEEN |
| | EDILEDIVLTLTLFEDREMIEERLKTYAHLFDDKVMKQLKRRRYTGWGRLSRKLINGIRDKQSGKTIL |
| | DFLKSDGFANRNFMQLIHDDSLTFKEDIQKAQVSGQGDSLHEHIANLAGSPAIKKGILQTVKVVDELV |
| | KVMGRHKPENIVIEMARENQTTQKGQKNSRERMKRIEEGIKELGSQILKEHPVENTQLQNEKLYLYYL |
| | QNGRDMYVDQELDINRLSDYDVDAIVPQSFLKDDSIDNKVLTRSDKNRGKSDNVPSEEVVKKMKNYWR |
| | QLLNAKLITQRKFDNLTKAERGGLSELDKAGFIKRQLVETRQITKHVAQILDSRMNTKYDENDKLIRE |
| | VKVITLKSKLVSDFRKDFQFYKVREINNYHHAHDAYLNAVVGTALIKKYPKLESEFVYGDYKVYDVRK |
| | MIAKSEQEIGKATAKYFFYSNIMNFFKTEITLANGEIRKRPLIETNGETGEIVWDKGRDFATVRKVLS |
| | MPQVNIVKKTEVQTGGFSKESILPKRNSDKLIARKKDWDPKKYGGFDSPTVAYSVLVVAKVEKGKSKK |
| | LKSVKELLGITIMERSSFEKNPIDFLEAKGYKEVKKDLIIKLPKYSLFELENGRKRMLASAGELQKGN |
| | ELALPSKYVNFLYLASHYEKLKGSPEDNEQKQLFVEQHKHYLDEIIEQISEFSKRVILADANLDKVLS |
| | AYNKHRDKPIREQAENIIHLFTLTNLGAPAAFKYFDTTIDRKRYTSTKEVLDATLIHQSITGLYETRI |
| | DLSQLGGDSGGSSGGSKRTADGSEFESPKKKRKVSGGSSGGSVISYDNYVTILDEETLKAWIAKLEKA |
| | PVFAFATATDSLDNISANLVGLSFAIEPGVAAYIPVAHDYLDAPDQISRERALELLKPLLEDEKALKV |
| | GQNLKYDRGILANYGIELRGIAFDTMLESYILNSVAGRHDMDSLAERWLKHKTITFEEIAGKGKNQLT |
| | FNQIALEEAGRYAAEDADVTLQLHLKMWPDLQKHKGPLNVFENIEMPLVPVLSRIERNGVKIDPKVLH |
| | NHSEELTLRLAELEKKAHEIAGEEFNLSSTKQLQTILFEKQGIKPLKKTPGGAPSTSEEVLEELALDY |
| | PLPKVILEYRGLAKLKSTYTDKLPLMINPKTGRVHTSYHQAVTATGRLSSTDPNLQNIPVRNEEGRRI |
| | RQAFIAPEDYVIVSADYSQIELRIMAHLSRDKGLLTAFAEGKDIHRATAAEVFGLPLETVTSEQRRSA |
| | KAINFGLIYGMSAFGLARQLNIPRKEAQKYMDLYFERYPGVLEYMERTRAQAKEQGYVETLDGRRLYL |
| | PDIKSSNGARRAAAERAAINAPMQGTAADIIKRAMIAVDAWLQAEQPRVRMIMQVHDELVFEVHKDDV |
| | DAVAKQIHQLMENCTRLDVPLLVEVGSGENWDQAHSGGSSGSETPGTSESATPESSGGSSPSKKNGRS |
| | GPQPHKRWVFTLNNPSEDERKKIRDLPISLFDYFIVGEEGNEEGRTPHLQGFANFVKKQTFNKVKWYL |
| | GARCHIEKAKGTDQQNKEYCSKEGNLLMECGAPRSQGQR (SEQ ID NO: 204) |
| pCMV- | MDKKYSIGLDIGTNSVGWAVITDEYKVPSKKFKVLGNTDRHSIKKNLIGALLFDSGETAEATRLKRTA |
| T7- | RRRYTRRKNRICYLQEIFSNEMAKVDDSFFHRLEESFLVEEDKKHERHPIFGNIVDEVAYHEKYPTIY |
| nCas9- | HLRKKLVDSTDKADLRLIYLALAHMIKFRGHFLIEGDLNPDNSDVDKLFIQLVQTYNQLFEENPINAS |
| PCV2- | GVDAKAILSARLSKSRRLENLIAQLPGEKKNGLFGNLIALSLGLTPNFKSNFDLAEDAKLQLSKDTYD |
| Ecklenow | DDLDNLLAQIGDQYADLFLAAKNLSDAILLSDILRVNTEITKAPLSASMIKRYDEHHQDLTLLKALVR |
| | QQLPEKYKEIFFDQSKNGYAGYIDGGASQEEFYKFIKPILEKMDGTEELLVKLNREDLLRKQRTFDNG |
| | SIPHQIHLGELHAILRRQEDFYPFLKDNREKIEKILTFRIPYYVGPLARGNSRFAWMTRKSEETITPW |
| | NFEEVVDKGASAQSFIERMTNFDKNLPNEKVLPKHSLLYEYFTVYNELTKVKYVTEGMRKPAFLSGEQ |
| | KKAIVDLLFKTNRKVTVKQLKEDYFKKIECFDSVEISGVEDRFNASLGTYHDLLKIIKDKDFLDNEEN |
| | EDILEDIVLTLTLFEDREMIEERLKTYAHLFDDKVMKQLKRRRYTGWGRLSRKLINGIRDKQSGKTIL |
| | DFLKSDGFANRNFMQLIHDDSLTFKEDIQKAQVSGQGDSLHEHIANLAGSPAIKKGILQTVKVVDELV |
| | KVMGRHKPENIVIEMARENQTTQKGQKNSRERMKRIEEGIKELGSQILKEHPVENTQLQNEKLYLYYL |
| | QNGRDMYVDQELDINRLSDYDVDAIVPQSFLKDDSIDNKVLTRSDKNRGKSDNVPSEEVVKKMKNYWR |
| | QLLNAKLITQRKFDNLTKAERGGLSELDKAGFIKRQLVETRQITKHVAQILDSRMNTKYDENDKLIRE |
| | VKVITLKSKLVSDFRKDFQFYKVREINNYHHAHDAYLNAVVGTALIKKYPKLESEFVYGDYKVYDVRK |
| | MIAKSEQEIGKATAKYFFYSNIMNFFKTEITLANGEIRKRPLIETNGETGEIVWDKGRDFATVRKVLS |
| | MPQVNIVKKTEVQTGGFSKESILPKRNSDKLIARKKDWDPKKYGGFDSPTVAYSVLVVAKVEKGKSKK |
| | LKSVKELLGITIMERSSFEKNPIDFLEAKGYKEVKKDLIIKLPKYSLFELENGRKRMLASAGELQKGN |
| | ELALPSKYVNFLYLASHYEKLKGSPEDNEQKQLFVEQHKHYLDEIIEQISEFSKRVILADANLDKVLS |
| | AYNKHRDKPIREQAENIIHLFTLTNLGAPAAFKYFDTTIDRKRYTSTKEVLDATLIHQSITGLYETRI |
| | DLSQLGGDSGGSSGSETPGTSESATPESSGGSSPSKKNGRSGPQPHKRWVFTLNNPSEDERKKIRDLP |
| | ISLFDYFIVGEEGNEEGRTPHLQGFANFVKKQTFNKVKWYLGARCHIEKAKGTDQQNKEYCSKEGNLL |
| | MECGAPRSQGQRSGGSSGGSKRTADGSEFESPKKKRKVSGGSSGGSVISYDNYVTILDEETLKAWIAK |
| | LEKAPVFAFATATDSLDNISANLVGLSFAIEPGVAAYIPVAHDYLDAPDQISRERALELLKPLLEDEK |
| | ALKVGQNLKYDRGILANYGIELRGIAFDTMLESYILNSVAGRHDMDSLAERWLKHKTITFEEIAGKGK |
| | NQLTFNQIALEEAGRYAAEDADVTLQLHLKMWPDLQKHKGPLNVFENIEMPLVPVLSRIERNGVKIDP |
| | KVLHNHSEELTLRLAELEKKAHEIAGEEFNLSSTKQLQTILFEKQGIKPLKKTPGGAPSTSEEVLEEL |
| | ALDYPLPKVILEYRGLAKLKSTYTDKLPLMINPKTGRVHTSYHQAVTATGRLSSTDPNLQNIPVRNEE |
| | GRRIRQAFIAPEDYVIVSADYSQIELRIMAHLSRDKGLLTAFAEGKDIHRATAAEVFGLPLETVTSEQ |
| | RRSAKAINFGLIYGMSAFGLARQLNIPRKEAQKYMDLYFERYPGVLEYMERTRAQAKEQGYVETLDGR |
| | RLYLPDIKSSNGARRAAAERAAINAPMQGTAADIIKRAMIAVDAWLQAEQPRVRMIMQVHDELVFEVH |
| | KDDVDAVAKQIHQLMENCTRLDVPLLVEVGSGENWDQAHSGGSKRTADGSEFESPKKKRKV (SEQ |
| | ID NO: 205) |
| pCMV- | MSPSKKNGRSGPQPHKRWVFTLNNPSEDERKKIRDLPISLFDYFIVGEEGNEEGRTPHLQGFANFVKK |
| T7- | QTFNKVKWYLGARCHIEKAKGTDQQNKEYCSKEGNLLMECGAPRSQGQRSGGSSGSETPGTSESATPE |
| PCV2- | SSGGSVISYDNYVTILDEETLKAWIAKLEKAPVFAFATATDSLDNISANLVGLSFAIEPGVAAYIPVA |
| Ecklenow- | HDYLDAPDQISRERALELLKPLLEDEKALKVGQNLKYDRGILANYGIELRGIAFDTMLESYILNSVAG |
| nCas9 | RHDMDSLAERWLKHKTITFEEIAGKGKNQLTFNQIALEEAGRYAAEDADVTLQLHLKMWPDLQKHKGP |
| | LNVFENIEMPLVPVLSRIERNGVKIDPKVLHNHSEELTLRLAELEKKAHEIAGEEFNLSSTKQLQTIL |
| | FEKQGIKPLKKTPGGAPSTSEEVLEELALDYPLPKVILEYRGLAKLKSTYTDKLPLMINPKTGRVHTS |
| | YHQAVTATGRLSSTDPNLQNIPVRNEEGRRIRQAFIAPEDYVIVSADYSQIELRIMAHLSRDKGLLTA |
| | FAEGKDIHRATAAEVFGLPLETVTSEQRRSAKAINFGLIYGMSAFGLARQLNIPRKEAQKYMDLYFER |
| | YPGVLEYMERTRAQAKEQGYVETLDGRRLYLPDIKSSNGARRAAAERAAINAPMQGTAADIIKRAMIA |
| | VDAWLQAEQPRVRMIMQVHDELVFEVHKDDVDAVAKQIHQLMENCTRLDVPLLVEVGSGENWDQAHSG |
| | GSSGGSKRTADGSEFESPKKKRKVSGGSSGGSDKKYSIGLDIGTNSVGWAVITDEYKVPSKKFKVLGN |
| | TDRHSIKKNLIGALLFDSGETAEATRLKRTARRRYTRRKNRICYLQEIFSNEMAKVDDSFFHRLEESF |
| | LVEEDKKHERHPIFGNIVDEVAYHEKYPTIYHLRKKLVDSTDKADLRLIYLALAHMIKFRGHFLIEGD |
| | LNPDNSDVDKLFIQLVQTYNQLFEENPINASGVDAKAILSARLSKSRRLENLIAQLPGEKKNGLFGNL |
| | IALSLGLTPNFKSNFDLAEDAKLQLSKDTYDDDLDNLLAQIGDQYADLFLAAKNLSDAILLSDILRVN |
| | TEITKAPLSASMIKRYDEHHQDLTLLKALVRQQLPEKYKEIFFDQSKNGYAGYIDGGASQEEFYKFIK |
| | PILEKMDGTEELLVKLNREDLLRKQRTFDNGSIPHQIHLGELHAILRRQEDFYPFLKDNREKIEKILT |
| | FRIPYYVGPLARGNSRFAWMTRKSEETITPWNFEEVVDKGASAQSFIERMTNFDKNLPNEKVLPKHSL |
| | LYEYFTVYNELTKVKYVTEGMRKPAFLSGEQKKAIVDLLFKTNRKVTVKQLKEDYFKKIECFDSVEIS |
| | GVEDRFNASLGTYHDLLKIIKDKDFLDNEENEDILEDIVLTLTLFEDREMIEERLKTYAHLFDDKVMK |
| | QLKRRRYTGWGRLSRKLINGIRDKQSGKTILDFLKSDGFANRNFMQLIHDDSLTFKEDIQKAQVSGQG |
| | DSLHEHIANLAGSPAIKKGILQTVKVVDELVKVMGRHKPENIVIEMARENQTTQKGQKNSRERMKRIE |
| | EGIKELGSQILKEHPVENTQLQNEKLYLYYLQNGRDMYVDQELDINRLSDYDVDAIVPQSFLKDDSID |
| | NKVLTRSDKNRGKSDNVPSEEVVKKMKNYWRQLLNAKLITQRKFDNLTKAERGGLSELDKAGFIKRQL |
| | VETRQITKHVAQILDSRMNTKYDENDKLIREVKVITLKSKLVSDFRKDFQFYKVREINNYHHAHDAYL |
| | NAVVGTALIKKYPKLESEFVYGDYKVYDVRKMIAKSEQEIGKATAKYFFYSNIMNFFKTEITLANGEI |
| | RKRPLIETNGETGEIVWDKGRDFATVRKVLSMPQVNIVKKTEVQTGGFSKESILPKRNSDKLIARKKD |
| | WDPKKYGGFDSPTVAYSVLVVAKVEKGKSKKLKSVKELLGITIMERSSFEKNPIDFLEAKGYKEVKKD |
| | LIIKLPKYSLFELENGRKRMLASAGELQKGNELALPSKYVNFLYLASHYEKLKGSPEDNEQKQLFVEQ |
| | HKHYLDEIIEQISEFSKRVILADANLDKVLSAYNKHRDKPIREQAENIIHLFTLTNLGAPAAFKYFDT |
| | TIDRKRYTSTKEVLDATLIHQSITGLYETRIDLSQLGGDSGGSKRTADGSEFESPKKKRKV (SEQ |
| | ID NO: 206) |
| pCMV- | MVISYDNYVTILDEETLKAWIAKLEKAPVFAFATATDSLDNISANLVGLSFAIEPGVAAYIPVAHDYL |
| T7- | DAPDQISRERALELLKPLLEDEKALKVGQNLKYDRGILANYGIELRGIAFDTMLESYILNSVAGRHDM |
| Ecklenow- | DSLAERWLKHKTITFEEIAGKGKNQLTFNQIALEEAGRYAAEDADVTLQLHLKMWPDLQKHKGPLNVF |
| PCV2- | ENIEMPLVPVLSRIERNGVKIDPKVLHNHSEELTLRLAELEKKAHEIAGEEFNLSSTKQLQTILFEKQ |
| nCas9 | GIKPLKKTPGGAPSTSEEVLEELALDYPLPKVILEYRGLAKLKSTYTDKLPLMINPKTGRVHTSYHQA |
| | VTATGRLSSTDPNLQNIPVRNEEGRRIRQAFIAPEDYVIVSADYSQIELRIMAHLSRDKGLLTAFAEG |
| | KDIHRATAAEVFGLPLETVTSEQRRSAKAINFGLIYGMSAFGLARQLNIPRKEAQKYMDLYFERYPGV |
| | LEYMERTRAQAKEQGYVETLDGRRLYLPDIKSSNGARRAAAERAAINAPMQGTAADIIKRAMIAVDAW |
| | LQAEQPRVRMIMQVHDELVFEVHKDDVDAVAKQIHQLMENCTRLDVPLLVEVGSGENWDQAHSGGSSG |
| | GSKRTADGSEFESPKKKRKVSGGSSGGSSPSKKNGRSGPQPHKRWVFTLNNPSEDERKKIRDLPISLF |
| | DYFIVGEEGNEEGRTPHLQGFANFVKKQTFNKVKWYLGARCHIEKAKGTDQQNKEYCSKEGNLLMECG |
| | APRSQGQRSGGSSGSETPGTSESATPESSGGSDKKYSIGLDIGTNSVGWAVITDEYKVPSKKFKVLGN |
| | TDRHSIKKNLIGALLFDSGETAEATRLKRTARRRYTRRKNRICYLQEIFSNEMAKVDDSFFHRLEESF |
| | LVEEDKKHERHPIFGNIVDEVAYHEKYPTIYHLRKKLVDSTDKADLRLIYLALAHMIKFRGHFLIEGD |
| | LNPDNSDVDKLFIQLVQTYNQLFEENPINASGVDAKAILSARLSKSRRLENLIAQLPGEKKNGLFGNL |
| | IALSLGLTPNFKSNFDLAEDAKLQLSKDTYDDDLDNLLAQIGDQYADLFLAAKNLSDAILLSDILRVN |
| | TEITKAPLSASMIKRYDEHHQDLTLLKALVRQQLPEKYKEIFFDQSKNGYAGYIDGGASQEEFYKFIK |
| | PILEKMDGTEELLVKLNREDLLRKQRTFDNGSIPHQIHLGELHAILRRQEDFYPFLKDNREKIEKILT |
| | FRIPYYVGPLARGNSRFAWMTRKSEETITPWNFEEVVDKGASAQSFIERMTNFDKNLPNEKVLPKHSL |
| | LYEYFTVYNELTKVKYVTEGMRKPAFLSGEQKKAIVDLLFKTNRKVTVKQLKEDYFKKIECFDSVEIS |
| | GVEDRFNASLGTYHDLLKIIKDKDFLDNEENEDILEDIVLTLTLFEDREMIEERLKTYAHLFDDKVMK |
| | QLKRRRYTGWGRLSRKLINGIRDKQSGKTILDFLKSDGFANRNFMQLIHDDSLTFKEDIQKAQVSGQG |
| | DSLHEHIANLAGSPAIKKGILQTVKVVDELVKVMGRHKPENIVIEMARENQTTQKGQKNSRERMKRIE |
| | EGIKELGSQILKEHPVENTQLQNEKLYLYYLQNGRDMYVDQELDINRLSDYDVDAIVPQSFLKDDSID |
| | NKVLTRSDKNRGKSDNVPSEEVVKKMKNYWRQLLNAKLITQRKFDNLTKAERGGLSELDKAGFIKRQL |
| | VETRQITKHVAQILDSRMNTKYDENDKLIREVKVITLKSKLVSDFRKDFQFYKVREINNYHHAHDAYL |
| | NAVVGTALIKKYPKLESEFVYGDYKVYDVRKMIAKSEQEIGKATAKYFFYSNIMNFFKTEITLANGEI |
| | RKRPLIETNGETGEIVWDKGRDFATVRKVLSMPQVNIVKKTEVQTGGFSKESILPKRNSDKLIARKKD |
| | WDPKKYGGFDSPTVAYSVLVVAKVEKGKSKKLKSVKELLGITIMERSSFEKNPIDFLEAKGYKEVKKD |
| | LIIKLPKYSLFELENGRKRMLASAGELQKGNELALPSKYVNFLYLASHYEKLKGSPEDNEQKQLFVEQ |
| | HKHYLDEIIEQISEFSKRVILADANLDKVLSAYNKHRDKPIREQAENIIHLFTLTNLGAPAAFKYFDT |
| | TIDRKRYTSTKEVLDATLIHQSITGLYETRIDLSQLGGDSGGSKRTADGSEFESPKKKRKV (SEQ |
| | ID NO: 207) |
| pCMV- | MVISYDNYVTILDEETLKAWIAKLEKAPVFAFATATDSLDNISANLVGLSFAIEPGVAAYIPVAHDYL |
| T7- | DAPDQISRERALELLKPLLEDEKALKVGQNLKYDRGILANYGIELRGIAFDTMLESYILNSVAGRHDM |
| Ecklenow- | DSLAERWLKHKTITFEEIAGKGKNQLTFNQIALEEAGRYAAEDADVTLQLHLKMWPDLQKHKGPLNVF |
| nCas9- | ENIEMPLVPVLSRIERNGVKIDPKVLHNHSEELTLRLAELEKKAHEIAGEEFNLSSTKQLQTILFEKQ |
| PCV2 | GIKPLKKTPGGAPSTSEEVLEELALDYPLPKVILEYRGLAKLKSTYTDKLPLMINPKTGRVHTSYHQA |
| | VTATGRLSSTDPNLQNIPVRNEEGRRIRQAFIAPEDYVIVSADYSQIELRIMAHLSRDKGLLTAFAEG |
| | KDIHRATAAEVFGLPLETVTSEQRRSAKAINFGLIYGMSAFGLARQLNIPRKEAQKYMDLYFERYPGV |
| | LEYMERTRAQAKEQGYVETLDGRRLYLPDIKSSNGARRAAAERAAINAPMQGTAADIIKRAMIAVDAW |
| | LQAEQPRVRMIMQVHDELVFEVHKDDVDAVAKQIHQLMENCTRLDVPLLVEVGSGENWDQAHSGGSSG |
| | GSKRTADGSEFESPKKKRKVSGGSSGGSDKKYSIGLDIGTNSVGWAVITDEYKVPSKKFKVLGNTDRH |
| | SIKKNLIGALLFDSGETAEATRLKRTARRRYTRRKNRICYLQEIFSNEMAKVDDSFFHRLEESFLVEE |
| | DKKHERHPIFGNIVDEVAYHEKYPTIYHLRKKLVDSTDKADLRLIYLALAHMIKFRGHFLIEGDLNPD |
| | NSDVDKLFIQLVQTYNQLFEENPINASGVDAKAILSARLSKSRRLENLIAQLPGEKKNGLFGNLIALS |
| | LGLTPNFKSNFDLAEDAKLQLSKDTYDDDLDNLLAQIGDQYADLFLAAKNLSDAILLSDILRVNTEIT |
| | KAPLSASMIKRYDEHHQDLTLLKALVRQQLPEKYKEIFFDQSKNGYAGYIDGGASQEEFYKFIKPILE |
| | KMDGTEELLVKLNREDLLRKQRTFDNGSIPHQIHLGELHAILRRQEDFYPFLKDNREKIEKILTFRIP |
| | YYVGPLARGNSRFAWMTRKSEETITPWNFEEVVDKGASAQSFIERMTNFDKNLPNEKVLPKHSLLYEY |
| | FTVYNELTKVKYVTEGMRKPAFLSGEQKKAIVDLLFKTNRKVTVKQLKEDYFKKIECFDSVEISGVED |
| | RFNASLGTYHDLLKIIKDKDFLDNEENEDILEDIVLTLTLFEDREMIEERLKTYAHLFDDKVMKQLKR |
| | RRYTGWGRLSRKLINGIRDKQSGKTILDFLKSDGFANRNFMQLIHDDSLTFKEDIQKAQVSGQGDSLH |
| | EHIANLAGSPAIKKGILQTVKVVDELVKVMGRHKPENIVIEMARENQTTQKGQKNSRERMKRIEEGIK |
| | ELGSQILKEHPVENTQLQNEKLYLYYLQNGRDMYVDQELDINRLSDYDVDAIVPQSFLKDDSIDNKVL |
| | TRSDKNRGKSDNVPSEEVVKKMKNYWRQLLNAKLITQRKFDNLTKAERGGLSELDKAGFIKRQLVETR |
| | QITKHVAQILDSRMNTKYDENDKLIREVKVITLKSKLVSDFRKDFQFYKVREINNYHHAHDAYLNAVV |
| | GTALIKKYPKLESEFVYGDYKVYDVRKMIAKSEQEIGKATAKYFFYSNIMNFFKTEITLANGEIRKRP |
| | LIETNGETGEIVWDKGRDFATVRKVLSMPQVNIVKKTEVQTGGFSKESILPKRNSDKLIARKKDWDPK |
| | KYGGFDSPTVAYSVLVVAKVEKGKSKKLKSVKELLGITIMERSSFEKNPIDFLEAKGYKEVKKDLIIK |
| | LPKYSLFELENGRKRMLASAGELQKGNELALPSKYVNFLYLASHYEKLKGSPEDNEQKQLFVEQHKHY |
| | LDEIIEQISEFSKRVILADANLDKVLSAYNKHRDKPIREQAENIIHLFTLTNLGAPAAFKYFDTTIDR |
| | KRYTSTKEVLDATLIHQSITGLYETRIDLSQLGGDSGGSSGSETPGTSESATPESSGGSSPSKKNGRS |
| | GPQPHKRWVFTLNNPSEDERKKIRDLPISLFDYFIVGEEGNEEGRTPHLQGFANFVKKQTFNKVKWYL |
| | GARCHIEKAKGTDQQNKEYCSKEGNLLMECGAPRSQGQR (SEQ ID NO: 208) |
| T7- | MKRTADGSEFESPKKKRKVSGGSRALVVIRLSRVTDATTSPERQLESCQQLCAQRGWDVVGVAEDLDV |
| 5′UTR- | SGAVDPFDRKRRPNLARWLAFEEQPFDVIVAYRVDRLTRSIRHLQQLVHWAEDHKKLVVSATEAHFDT |
| NLS- | TTPFAAVVIALMGTVAQMELEAIKERNRSAAHFNIRAGKYRGSLPPWGYLPTRVDGEWRLVPDPVQRE |
| BxbI- | RILEVYHRVVDNHEPLHLVAHDLNRRGVLSPKDYFAQLQGREPQGREWSATALKRSMISEAMLGYATL |
| 3′UTR- | NGKTVRDDDGAPLVRAEPILTREQLEALRAELVKTSRAKPAVSTPSLLLRVLFCAVCGEPAYKFAGGG |
| polyA | RKHPRYRCRSMGFPKHCGNGTVAMAEWDAFCEEQVLDLLGDAERLEKVWVAGSDSAVELAEVNAELVD |
| | LTSLIGSPAYRAGSPQREALDARIAALAARQEELEGLEARPSGWEWRETGQRFGDWWREQDTAAKNTW |
| | LRSMNVRLTFDVRGGLTRTIDFGDLQEYEQHLRLGSVVERLHTGMS (SEQ ID NO: 209) |

### EXAMPLES

The invention is further described in the following examples, which do not limit the scope of the invention described in the claims.

### Methods

The following materials and methods were used in the examples below.

### Plasmids and Oligonucleotides

Plasmids were generated via isothermal assembly or Golden Gate assembly (Table 1). Expression plasmids for human U6 promoter-driven gRNAs were generated by annealing and ligating duplexed oligonucleotides corresponding to spacer sequences into BsmBI-digested BPK1520 (Addgene plasmid 65777)⁶². Target site sequences for gRNAs are available in Table 2. Oligonucleotides used in this study for amplicon sequencing (Table 3) and clkDNA oligonucleotides (Tables 3-4) were purchased from Integrated DNA Technologies (IDT); gene fragments were ordered from Twist Biosciences.

### Human Cell Culture

Human HEK 293T cells (ATCC) were cultured at 37° C. with 5% CO₂ in Dulbecco's modified Eagle medium supplemented with 10% heat-inactivated fetal bovine serum and 1% penicillin-streptomycin (ThermoFisher). HCT-116 cells (ATCC) were cultured at 37° C. with 5% CO₂ in McCoy's medium supplemented with 10% heat-inactivated fetal bovine serum and 1% penicillin-streptomycin (ThermoFisher). Primary human fibroblasts were obtained via donor skin biopsies under written informed consent approved by the Massachusetts General Hospital institutional ethics review board (protocol number 2007P002248). Biopsies were collected into a transport medium of DMEM (Gibco) with 1% Penicillin-Streptomycin-L-Glutamine (Corning) and washed twice in PBS (Gibco) with 10% Penicillin-Streptomycin-L-Glutamine. Dissected sections of the biopsy were plated with dermis side down into tissue culture dishes and incubated at 37° C. for 15 min. DMEM with 10% FBS (Sigma) and 1% Penicillin-Streptomycin-Glutamine was gently added over the sections of skin to enable transformation. Primary human fibroblasts were cultured at 37° C. with 5% CO₂ in Dulbecco's modified Eagle medium (DMEM) supplemented with 10% heat-inactivated fetal bovine serum and 1% penicillin-streptomycin (ThermoFisher).

The supernatant medium from cell cultures was analyzed monthly for the presence of mycoplasma using MycoAlert PLUS (Lonza) or via polymerase chain reaction (PCR).

### PCE and LCE Transfection

All experiments were performed with at least 3 replicates; we define biological replicates as results from transfections performed using cells seeded from different passages of cells, and technical replicates as results obtained from transfections performed using the same set of seeded cells. Transfections were performed between 20 and 24 hours following seeding of ~2.2×10⁴ HEK 293T cells per well in 96-well plates. Standard transfections included 80 ng of CE expression plasmid, 25 ng of gRNA expression plasmid, 13 ng of nicking gRNA (ngRNA) expression plasmid, and either 16 or 12 pmol of clkDNA unless otherwise indicated (for more details, see Example 7). The DNA mixtures were mixed with TransIT-X2 (Mirus) at a ratio of 0.5 μL of TransIT-X2 per 100 ng of total DNA, in a total volume of 20 μL Opti-MEM (Thermo Fisher Scientific), following manufacturer recommended protocols. This TransIT-X2:DNA solution was mixed gently (very brief low speed vortexing, as aggressive vortexing can negatively impact TransIT-X2:DNA complexing), was incubated for 15 minutes at room temperature, and then gently distributed across the seeded HEK 293T cells, taking care to follow the manufacturer recommendations for preparing the TransIT-X2:DNA complexes (including not leaving the TransIT-X2:DNA complexes in solution for longer than the manufacturer recommended times (e.g. ensuring <30 minutes), pipetting gently to mix the TransIT-X2 and DNA solutions together, and only gently spinning the mixed complexes in a centrifuge for a brief period of time).

HEK 293 cells were transfected between 20-24 hours following seeding of ~2.2×10⁴ cells per well in 96-well plates. Standard HEK 293 transfections included 60 ng of CE expression plasmid, 18.75 ng of gRNA expression plasmid, 9.75 ng of ngRNA expression plasmid, and 9 pmol of clkDNA. The DNA mixtures were mixed with 0.5 μL of TransIT-X2 per 100 ng of total DNA in a total volume of 20 μL Opti-MEM following the protocol described above. HeLa cells were transfected 20-24 hours following seeding of ~8×10³ cells per well in 96-well plates. Standard HeLa transfections included 40 ng of CE expression plasmid, 12.5 ng of gRNA expression plasmid, 6.5 ng of ngRNA expression plasmid, and 6 pmol of clkDNA, which were then mixed with 0.5 μL of TransIT-X2 per 100 ng of total DNA in a total volume of 20 μL Opti-MEM following the procedure described above. For U2OS, 3.5×10⁴ cells were seeded per well in 48-well plates ~20-24 hours prior to transfection. Standard U2OS transfections included 120 ng of CE expression plasmid, 37.5 ng of gRNA expression plasmid, 19.5 ng of ngRNA expression plasmid, and 18 pmol of clkDNA, which were then mixed with 0.5 μL of TransIT-X2 per 100 ng of total DNA in a total volume of 50 μL Opti-MEM following the procedure described above.

### Generation of Stable Primary Human Fibroblast CE Cell Line for Click Editing

Lentiviral vectors encoding the PCV2-nCas9-EcKlenow CE1 construct were produced in HEK 293T cells upon transfection with a packaging plasmid (psPAX2; Addgene plasmid 12260), an envelope plasmid (pMD2.G; Addgene plasmid 12259), as well as the CE1 expression plasmid (LTR-pEF1a-XTEN-nSpCas9-BPNLS-EcKlenow(-exo)-BPNLS; DRR921). Six hours post-transfection, HEK 293T cells were washed with PBS and incubated with new culture media for 48-72 hours. Lentiviral vectors were isolated through differential centrifugation by collecting conditioned media, centrifuging at 300 g for 10 min, followed by a 10 min 2,000 g spin to remove cells and cell debris. Lentiviral vectors were concentrated through ultracentrifugation at 70,000 g, and the vector pellet was resuspended using iced PBS. Human fibroblasts were stably transduced with lentiviral vectors encoding CE1 and further selected with puromycin (2 g/mL) over two passages.

Nucleofection was performed using the 4D-Nucleofector X Unit (Lonza) and P3 Primary Cell Kit, following the manufacturer's recommendations. For each nucleofection, 20 μL of nucleofector solution P3 was utilized to suspend ~2×10⁵ pelleted cells. The mixture was then combined with 680 ng of gRNA expression plasmid, 353.6 ng of the ngRNA expression plasmid, and 336 pmol clkDNA. Cells were transferred into a 16-well cuvette and electroporated using the CA-137 program. Immediately after pulsing, cells were recovered in pre-warmed Dulbecco's Modified Eagle's Medium (DMEM, Gibco) supplemented with 20% fetal bovine serum (FBS) and 1% of penicillin/streptomycin, transferred to a 48-well flat-bottom plate, and incubated at 37° C. under 5% CO2 for 72 hours prior to genomic DNA isolation.

mRNA Production and Transfections

An in vitro transcription template was cloned to encode the human codon optimized PCV2-nCas9-EcKlenow click editor (CE1) coding sequence into pIVT-nCas9(H840A) (Addgene plasmid 207455)(Liu et al., Nat Biotechnol. 2023 Sep. 14. doi: 10.1038/s41587-023-01947-w), which is a vector containing a CleanCap reagent AG-compatible T7 promoter backbone, 5′ and 3′ UTRs, and a 110-nt poly(A) (SEQ ID NO: 197) tract. The cloned plasmid was linearized with Esp3I (NEB) and column purified with a QIAquick PCR purification kit (Qiagen). The linearized template was transcribed using a HiScribe T7 mRNA Kit with Cleancap Reagent AG (NEB) with replacement of UTP by N1-methylpseudo-UTP (Trilink Biotechnologies) according to the manufacturer's instructions. The resulting mRNA was purified using a Monarch RNA Cleanup Kit (NEB).

HEK 293T, HCT116, or HeLa cells were seeded as described above in 96-well plates. For HEK 239T and HCT116 cells, 100 ng of mRNA and 10 pmol each of clkDNA, primary gRNA, and nicking gRNA was transfected 20 hrs after seeding using 0.7 μL of TransIT-mRNA and 0.7 μL of Boost reagent (Mirus Bio). For HeLa cells, 100 ng of mRNA and 5 pmol each of clkDNA, primary sgRNA, and nicking sgRNA were transfected 20 hrs after seeding using 0.5 μL of TransIT-mRNA and 0.5 μL of Boost reagent.

### Amplicon Sequencing and Data Analysis

Genomic DNA was harvested about 72 hours after transfection, by discarding the media, resuspending the cells in 100 μL of quick lysis buffer (20 mM Hepes pH 7.5, 100 mM KCl, 5 mM MgCl2, 5% glycerol, 25 mM DTT, 0.1% Triton X-100, and 60 ng/μL Proteinase K (NEB)), heating the lysate for 6 minutes at 66° C., heating at 98° C. for 2 minutes. Following incubation, genomic DNA was purified using 0.8× ratio of paramagnetic beads, prepared as previously described^{63,64}. The efficiency of genome modification by CE editing was determined by next-generation sequencing using a 2-step PCR-based Illumina library construction method. Briefly, genomic loci were amplified from approximately 100 ng of genomic DNA using Q5 High-fidelity DNA Polymerase (NEB) and the primers listed in Table 3, with cycling conditions of 1 cycle at 98° C. for 2 min; 35 cycles of 98° C. for 10 sec, 65° C. for 10 sec, 72° C. for 20 sec; and 1 cycle of 72° C. for 1 min. PCR products were purified using paramagnetic beads at a ratio of 1.8×. Approximately 20 ng of purified PCR product was used as template for a second PCR (PCR-2) to add Illumina barcodes with adapter sequences using Q5 and the primers listed in Table 3, with cycling conditions of 1 cycle at 98° C. for 2 min; 10 cycles at 98° C. for 10 sec, 65° C. for 30 sec, 72° C. 30 sec; and 1 cycle at 72° C. for 5 min. PCR-2 products were pooled based on concentrations from capillary electrophoresis (QIAxcel, Qiagen). Final libraries were quantified by QUBIT dsDNA High Sensitivity assay (ThermoFisher) and sequenced on a MiSeq sequencer using a 300-cycle v2 kit (Illumina). On-target genome editing activities were determined from sequencing data using CRISPResso2⁶⁵.

### Next Generation Sequencing Data Analysis

Using CRISPResso2, amplicon sequences were aligned to a reference sequence in HDR mode using the intended editing outcome as the expected allele (-e) and the parameters “-q 30” and “-discard_indel_reads TRUE”. For each amplicon, the quantification window (-qwc) was defined as the entire sequence between sgRNA- and nicking sgRNA-directed cut sites plus an additional 10 bp on either side of each sgRNA nicking site. The same quantification window was used for each amplicon, whether or not a ngRNA was transfected. Editing efficiencies were quantified by determining: (# of reads aligned to HDR/number of total reads). Indel efficiencies were quantified as (number of discarded indel-containing reads/number of total reads). The analysis of experiments containing clkDNAs with MMR evading mutations was ran using CRISPResso2 in standard mode, providing the reference amplicon and the gRNA sequence, and using the same quantification window as in HDR mode.

Assessment of gRNA-Dependent Off-Targets

We examined putative gRNA-dependent off-target editing with CEs by first designing 12 off-target sites per primary gRNA using CasOFFinder²⁴ with search parameters of 1-3 mismatches, 20 nt spacer, NRG protospacer-adjacent motif (PAM), and no RNA or DNA bulge off-targets. When possible, to increase potential sensitivity to detect off-targets, the CasOFFinder output was currated to select off-target sites with minimal mismatches in seed region (~10 bp adjacent to PAM). Amplicon-specific primers to amplify off-target sites were designed using Primer3 with amplicon length between 150-250 bp, and off-target sequence dictated as the target region and ideal melting temperature between 63 and 68° C. The PCR-1 amplicon-specific primers were designed by adding Illumina adapter sequences to gene-specific sequences. Transfections for CEs were performed as described above; nuclease transfections contained 80 ng of SpCas9 nuclease or nickase expression plasmid and 25 ng of the primary gRNA. gDNA was harvested, amplicon PCRs were performed, sequencing libraries were prepared and sequenced as described above. For analysis, CRISPResso2 was run in standard mode and indels were calculated using the CRISPResso_quantification_of_editing_frequency.txt output as (SUM(Insertions, Deletions)−Insertions and Deletions)/(Reads_Total)*100. On target editing for the DNMT1, ACTB, and VEGFA targets was analyzed using CRISPREsso2 HDR mode.

### Assessment of Potential HUHe Off-Targets

HUH pseudo-site targets were designed using TagScan⁶⁶ to search for sites within the human reference genome with the PCV2 binding sequence (AAGTATTACCAGC (SEQ ID NO: 210)) within 20 bp of an NGG PAM, optimally placing the PCV2 binding site in the solvent-accessible PAM-distal region of the non-target strand (Table 5). Once putative target sites were identified, oligonucleotides were ordered for spacer sequences and gRNA plasmids were cloned as described above (Table 5). Transfections were performed as described above, containing 80 ng of enzyme expression plasmid (CE, CE-deadPCV2, CE-deadCas9, SpCas9(H840A), or SpCas9), 25 ng of HUH pseudo-site targeting gRNA plasmid, optionally, 12 pmol of clkDNA, and 0.5 μL/100 ng of TransIT-X2 were mixed into a total volume 20 μL of Opti-MEM, and transfections, gDNA preparation, and sequencing protocols were performed as described above.

### Example 1. Development of Polymerase Click Editors (PCEs)

To test the PCE concept, we constructed an initial expression plasmid consisting of (1) the HUH endonuclease from porcine circovirus type 2 (PCV2) and (2) the large fragment of Escherichia coli DNA polymerase I (containing mutations D355A and E357A that deactivate its 3′-5′ exonuclease domain; EcKlenow), both fused to nCas9 (H840A, capable of nicking the NTS) to create the PCV2-nCas9-EcKlenow polymerase click editor (PCE; FIG. 1B). We cloned two sgRNA expression plasmids containing spacers that target opposite strands of the DNMT1 locus in human cells, for the initial PCE nick and for the secondary nick to enhance edit incorporation (PCE2 design; FIG. 1C). To assess the performance of the PCE2 designs, we performed a titration of the clkDNA dosage to identify an optimal clkDNA amount that leads to maximum editing efficiency, while minimizing cellular toxicity and unwanted indels. We transfected HEK 293T cells in 96-well format with the PCE plasmid, the two sgRNAs, and a range of clkDNA amounts from 0.0625 pmol to 32 pmol (including a no-clkDNA control). The clkDNA template encodes a trinucleotide AGG deletion at the DNMT1 target site (FIG. 2A). To minimize degradation of the clkDNA by cellular exonucleases (e.g., TREX1), we also explored the use of two phosphothiorate (PS) linkages added between the last three 3′ bases on the clkDNA. As a control, we also transfected nCas9 alone with an equivalent clkDNA lacking the HUH-recognition motif.

To evaluate PCE editing efficiency and indels at the DNMT1 locus using PCE2, we extracted genomic DNA (gDNA) from transfected cells and performed next generation sequencing (NGS) on the region spanning the target site. We observed that the percentage of reads containing the precise AGG deletion (without other indels) increased with the amount of transfected clkDNA up until ~16 μmol, reaching peak editing efficiency at ~7% precise editing (FIG. 2A, 2B). Strikingly, minimal levels of editing occurred in the nCas9 control condition (no PCV2 or EcKlenow polymerase fused to nCas9), reaching a maximum of 0.12% with 16 pmols of clkDNA (FIG. 2A, 2b), suggesting that clkDNA tethering via the HUH domain, and/or recruitment of a DNA-dependent DNA polymerase, are critical to achieve higher levels of editing. Indel rates were higher in the nCas9 alone condition (~6% with 16 pmols clkDNA) compared to PCV2-nCas9-EcKlenow (~2.5% with 16 pmols clkDNA) (FIG. 2A). Next, we tested PCE2 with clkDNAs encoding an AGC insertion in the NOLC1 locus via a similar clkDNA titration as described for DNMT1. A separate control was included in this experiment, containing a PCE with a catalytically inactivated PCV2 domain, harboring a Y96F mutation (dPCV2). This experiment yielded similar conclusions to the titration at the DMNT1 locus, where higher doses of clkDNA led to superior overall editing (albeit at lower overall efficiencies compared to DNMT1; FIG. 2C). The dPCV2 control exhibited low levels of editing analogous to those observed with nCas9 alone, again suggesting the essentiality of clkDNA recruitment to the target site to achieve higher levels of editing. Together, these results demonstrate that PCEs can efficiently install small edits at target loci, and indicate different methods by which the technology can be optimized.

One approach to increase PCE editing efficiency would be to increase Cas9 binding affinity for its target site and/or improve NTS nicking efficiency. We constructed a PCE (similarly fused to PCV2 and EcKlenow) using a nCas9 additionally containing R221K and N394K mutations that have previously been shown to exhibit increased Cas9 editing efficiency in certain contexts^{18,60}. When testing this construct at DNMT1 with 16 pmol of clkDNA, we observed similar levels of editing with or without these two additional Cas9 mutations (FIG. 2D). Therefore, we proceeded with the PCE construct that did not contain these mutations. Additional or alternate activity enhancing mutations that improve SpCas9 editing efficiency (Table E) may be tested.

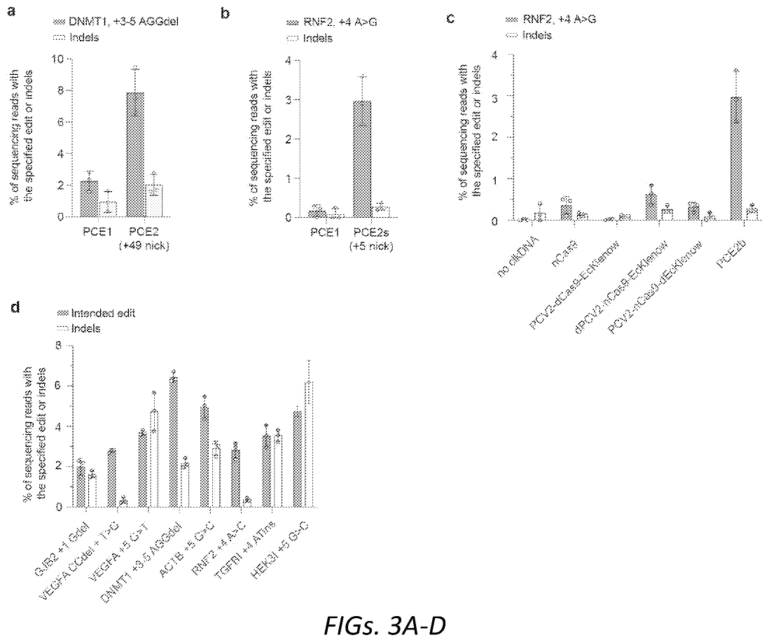

We directly compared PCE1 and PCE2 approaches (where PCE1 contains only the primary gRNA, PCE2 contains an additional gRNA to create a secondary nick to favor incorporation of the edit, and PCE2s (“spacer”) contains an additional gRNA whose spacer overlaps the edit so is less likely to nick the unedited allele) at two endogenous human loci in HEK 293T cells including in the DNMT1 gene (to create an +3-5AGG deletion) and in the RNF2 gene (to install a +4 A to G substitution). As expected, due to the secondary nick, the PCE2 or PCE2s approach led to higher editing efficiencies in both cases (FIGS. 3A and 3B). When using a set of controls that inactivated PCV2 (dPCV2), inactivated nCas9 (dCas9), or inactivated EcKlenow (dEcKlenow) in the PCE2 architecture, or when nCas9 alone was used (without any additional fusions), editing was either unobservable or suppressed when compared to the conventional PCE2 (FIG. 3C).

Next, we tested PCEs at additional genomic sites using clkDNAs encoding various other edits, including those that would generate (1) a 1 base pair (bp) deletion in the GJB2 gene to replicate a c35delG mutation, (2) a 2 bp deletion combined with a T-to-G transversion in the VEGFA gene (3) a 2 bp insertion in the TGFBI gene and (4) a G-to-C transversion at the HEK3 target site⁶⁷ (in the LINC01509 gene), in addition to repeating the 3 bp deletion in the DNMT1 gene and A-to-C transversion in the RNF2 gene (FIG. 3D). Across all target sites, we observed installation of the desired edit at varying efficiencies. Importantly, these levels of editing were achieved with PCEs with minimal PCE construct optimization (e.g., fusion points, linker lengths, standard domains without activity altering mutations, or other recruitment methods, etc.) and only a titration of clkDNAs bearing standard parameters (i.e. without thoroughly assessing the impact of the lengths of the FBR or PT of the clkDNA, as well as different chemical modifications for clkDNA protection from cellular exo- and endonucleases). Hence, further optimization of PCEs will likely lead to improved levels of editing and purity.

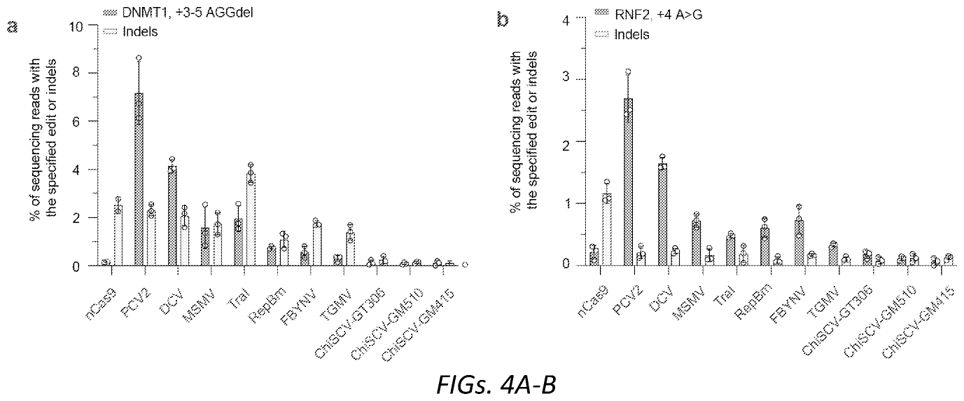

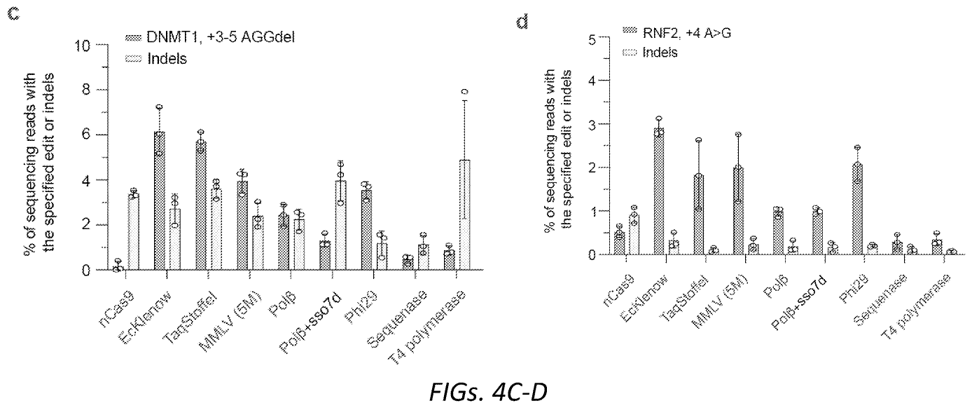

We then explored whether alternate HUH endonucleases may alter PCE efficiency. We tested a diversity of HUH rep and relaxase domains, including PCV2 (in our original design), DCV, MSMV, TraI, RepBm, FBYNV, TGMV, MSMV, and ChiSCV. Across two target sites, we observed that of the HUH enzymes tested, PCV2-containing PCE2 led to the highest editing efficiencies (FIG. 4A, 4B).

We then explored whether alternate polymerase domains may improve PCE efficiency. In addition to EcKlenow, we tested a diversity of DNA polymerases in the PCE architecture to generate clkDNA-encoded edits at the DNMT1 and HEK3 loci. The polymerases included Taq Stoffel fragment (StofTaq or TaqStoffel), a highly engineered MMLV reverse transcriptase used in prime editing³⁰ (but instead priming from a DNA:DNA template in this context), phi29 DNA polymerase, a modified version of T7 DNA polymerase (Sequenase)⁶⁸, T4 DNA polymerase, and human polymerase beta with or without Sso7d (shown to increase processivity of various polymerases⁴⁷). Across the two sites, we observed the highest levels of editing when using PCEs with wild-type EcKlenow (as in our original PCE design), wild-type StofTaq, Phi29, and the engineered MMLV RT (FIG. 4C, 4D). These results indicate that alternate polymerase domains may enhance PCE activity and that certain polymerases are less functional in the context of PCE fusions to Cas9.

### Example 2. PCEs for Targeted, in Cellulo Diversification

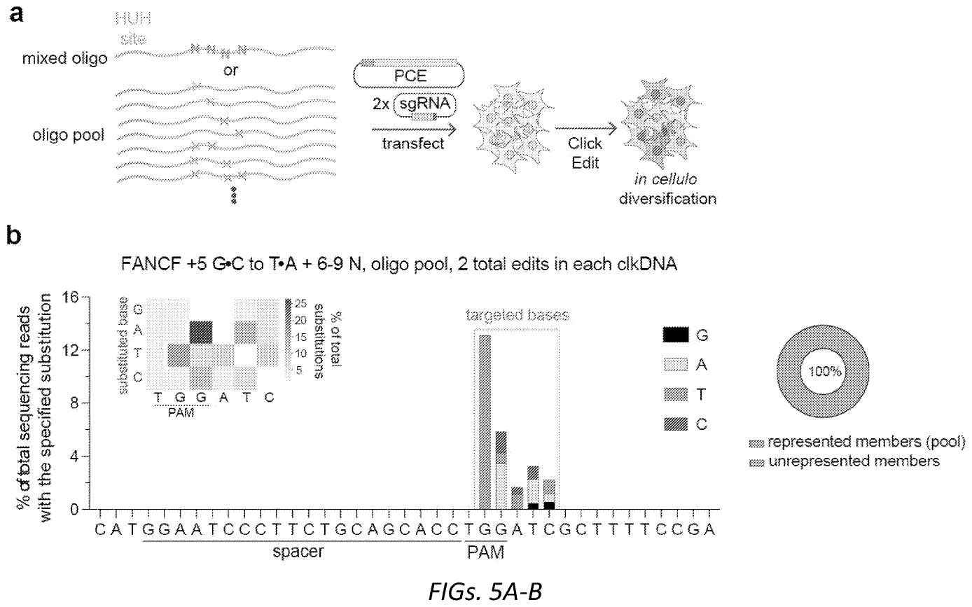

We hypothesized that PCEs would also be an innovative way to facilitate in cellulo, targeted diversification of native DNA sequences via use of simple pooled oligo libraries (FIG. 5A). Each member of the library is a clkDNA containing an HUH recognition sequence, FBR, and a PT containing various diversified edits within a desired target window. In addition to applications in forward genetics, deep mutational scanning, and drug screening, this method could also provide a pooled approach to identify optimal clkDNA designs for corrective edits (e.g. silent mutations, identification of the most efficiently installed edit, etc).

To pilot this approach, we pooled four PCE clkDNAs, where each clkDNA encoded a G to T PAM mutation and one additional edit from bases +6 to +9 bp of the nick site (covering all types of single base mutations in this window). This pool was then mixed equimolar and transfected into HEK 293T cells along with PCE and two sgRNAs targeting the FANCF locus. NGS analysis revealed targeted diversification at the target site precisely within the defined window and with 100% of library members represented (FIG. 5B).

### Example 3. Dual-Flap PCE (“Double Click”) for More Complex Edits

Deletion and/or replacement of larger segments of endogenous DNA (>30 nt) with user-defined DNA may facilitate numerous applications. Some examples include exon replacement or recoding (enabling a single editing approach capable of treating a larger portion of mutations simultaneously), precise pathogenic repeat deletion and recoding (i.e., to treat diseases caused by trinucleotide repeat expansions), and placement of naturally occurring or engineered recombinase attachment sites (i.e. attB or attP) into the genome, which can be combined with serine integrases to enable gene-sized insertions (see section “PCEs, LCEs, or dual-overhang ligation combined with serine recombinases for gene-sized DNA insertions”) (FIGS. 40A-B).

### Example 4. Development of Ligase Click Editors (LCEs)

To test the LCE2 strategy, we transfected HEK 293T cells with an LCE construct (PCV2-nCas9(H840A)-T4Ligase), two sgRNAs targeting the FANCF locus (a primary sgRNA and a nicking sgRNA that directs a Cas9 nick 48 bp downstream of the primary nick site—LCE2 strategy), and a clkDNA encoding a +5 G to T transversion on the attachment sequence. As a control, we scrambled the HUH recognition site on the clkDNA, which should prevent clkDNA localization to the target site. NGS results revealed 1.7% editing efficiency when using the clkDNA with the correct HUH recognition site and only 0.28% editing efficiency when using the scrambled site (FIG. 7C). Lack of LCE led to no editing. Thus, the LCE construct itself and clkDNA recruitment to the target site is required for editing.

### Example 5. Dual-Overhang Ligation-Based Approach for Larger DNA Edits

To explore larger sequence insertions in the ~50 nt range, we first generated an initial construct composed of nCas9 (H840A) fused to T4 DNA ligase via a linker (encoding a bipartite SV40 NLS). We also co-expressed two sgRNAs targeting the FANCF locus in human cells, programmed to enable LCE nicking on opposite strands at 48 bp apart. In this design, we did not fuse or express an HUH endonuclease (unlike LCEs for small edits) since the 5′ ends of the ligDNA are required for ligation. We designed a ligDNA composed of a 48 bp duplex of genome-orthogonal sequence, that additionally harbored 20 bp overhangs on both ends. Notably, 5′ ends were phosphorylated and 3′ ends contained two phosphorothioate (PS) linkages between two added thymine bases to reduce clkDNA degradation by cellular exonucleases.

We sought to programmably replace the endogenous 48 bp sequence at FANCF with our duplexed ligDNA. We transfected HEK 293T cells with the ligDNA and plasmids encoding nCas9(H840A) or nCas9-T4Ligase and both sgRNAs. Next-generation sequencing (NGS) of the targeted locus revealed 10.6% perfect sequence replacement and 9.1% indels with nCas9 (FIG. 8D). Constructs harboring the fused T4 ligase led to similar editing efficiencies (11.2%) but with decreased indels (4.9%), resulting in a 2-4 fold increase in editing purity (FIG. 8D). Use of a catalytically inactive T4 Ligase also decreased indels, potentially due to residual ssDNA binding capability of the ligase; however, this occurred with a small concurrent decrease in editing efficiency (FIG. 8D).

Next, we hypothesized that ligDNA localization to the target site might increase replacement efficiency. To do so, we fused monomeric streptavidin (mSA) to the N-terminal end of our nCas9-ligase (T4) construct and designed a ligDNA with biotin conjugated to one or both 3′ ends (FIG. 8E). Transfection of the biotin-conjugated ligDNA with plasmids encoding nCas9-T4Ligase, the two gRNAs followed by NGS revealed comparable replacement efficiencies regardless of mSA fusion and decreased purity in the LCE conditions (FIG. 8F) relative to the unrecruited clkDNA (FIG. 8D).

### Example 6. Click-to-Install Genome Editing Using RNA-Programmable DNA-Dependent Polymerases and HUH Endonucleases

We constructed an initial CE fusion protein (CE1) that combined the HUHe from porcine circovirus 2 (PCV2) and a 3′-5′ exonuclease-deficient Klenow fragment from E. coil DNA polymerase I (EcKlenow⁶⁹) with nCas9 (H840A) (FIGS. 12A and 12E). Transfections were performed using separate CE and single gRNA expression plasmids along with a clkDNA, an approach that we termed CE1 or CE1.n1 due to use of a single primary gRNA (where CE1 defines the CE enzyme and n1 defines the gRNA/nicking strategy). The clkDNAs encoded a 3-bp deletion at DNMT1 or an A-to-C transversion at RNF2 (FIGS. 12F and 12G, respectively), and were modified with two 3′ phosphorothioate (PS) linkages. Amplicon sequencing from experiments using CE1 revealed 3.33% and 0.18% precise editing at DNMT1 and RNF2, respectively, with minimal indel byproducts (FIGS. 12E-G).

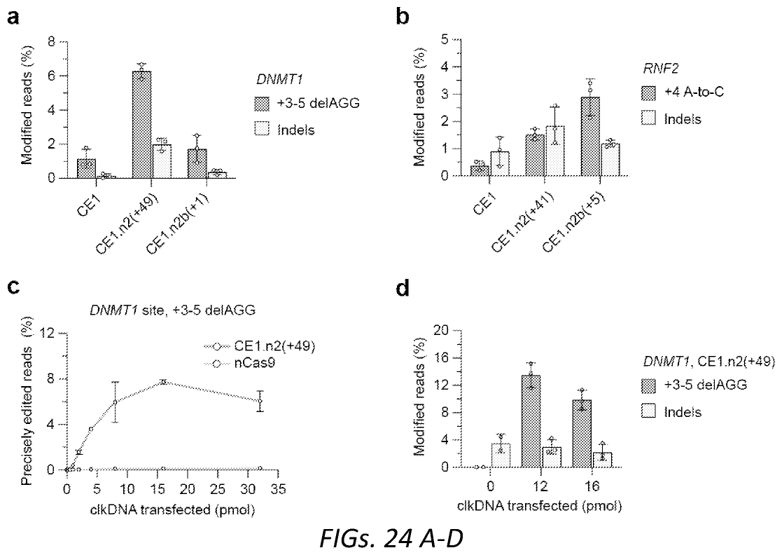

To improve edit efficiency and explore CE dependencies, we tested various gRNA and CE configurations. A secondary nick on the opposite strand should bias DNA repair to preferentially utilize the edited strand as the correct template, similar to BEs⁷⁰ and PEs²⁰. We performed experiments with CE1 and a secondary gRNA to direct CE-mediated nicking (ngRNA), leading to CE1.n2 or CE1.n2b conditions where the ngRNA is either distal from or overlaps the intended edit, respectively (FIG. 23). With CE1.n2 we observed ~3-fold increase in precise editing compared to CE1 at DNMT1, achieving 9.85% editing (FIG. 12F, FIG. 24A); a clkDNA titration using these conditions revealed that 16 pmol of clkDNA led to optimal editing efficiency in our initial experiments (FIGS. 24C-D, Example 7). When using an n2b approach at RNF2, we observed a ~18-fold increase in editing versus CE1, reaching 3.26% (FIG. 12G, FIG. 24B). Importantly, transfecting various control conditions nearly or fully abolished click editing, when using plasmids encoding nCas9 only (no DDP or HUHe fusions) or CE1.n2 containing catalytically inactive Cas9 (no NTS nick with HUH-less clkDNA), inactive PCV2 (diminished clkDNA recruitment), or inactive Klenow (attenuated polymerization) (FIGS. 12F,G, and Example 8). Notably, the n2b-mediated indels for RNF2 were lower compared to n2-induced indels at DNMT1, consistent with the hypothesis that ngRNAs that bind only after editing has occurred reduce the co-occurrence of nicks and the subsequent generation of DSBs (analogous to the PE2b strategy²⁰) (FIGS. 12F,G, and Example 8). These experiments demonstrate that CEs can achieve precise genome edits and that all components (nCas9, DDP, HUHe, and clkDNA) are required for productive click editing.

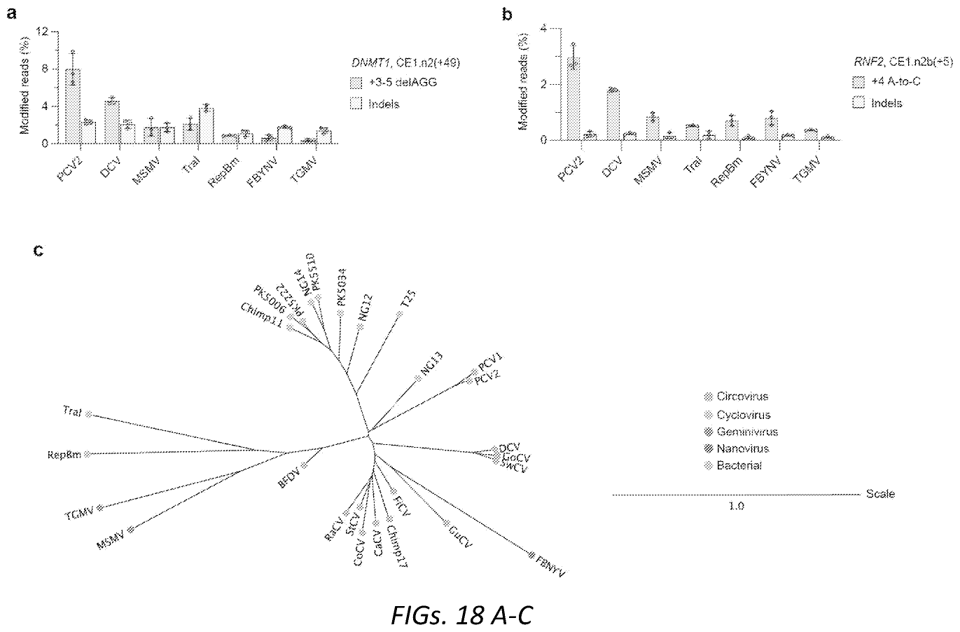

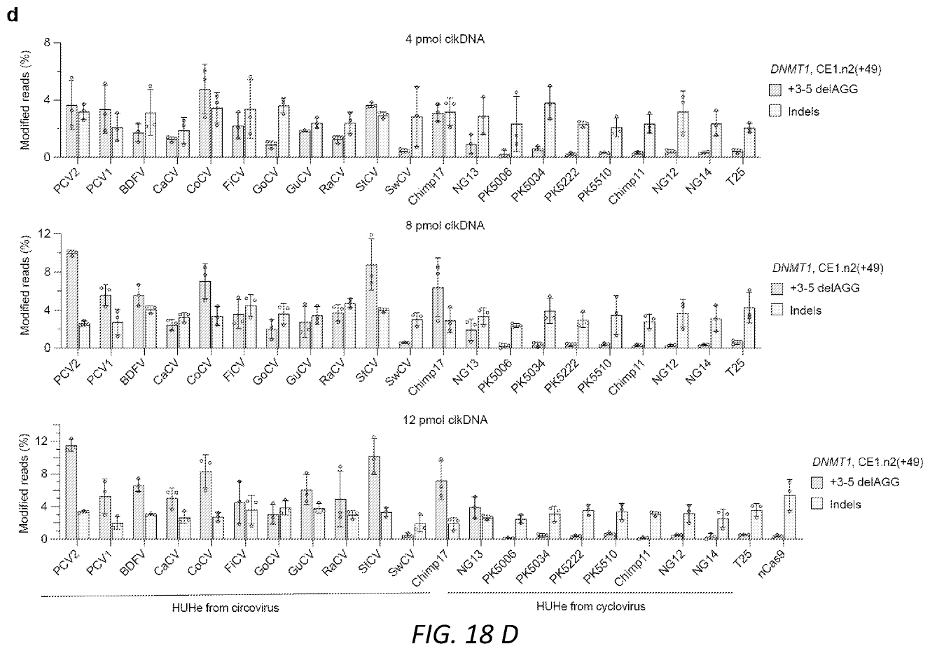

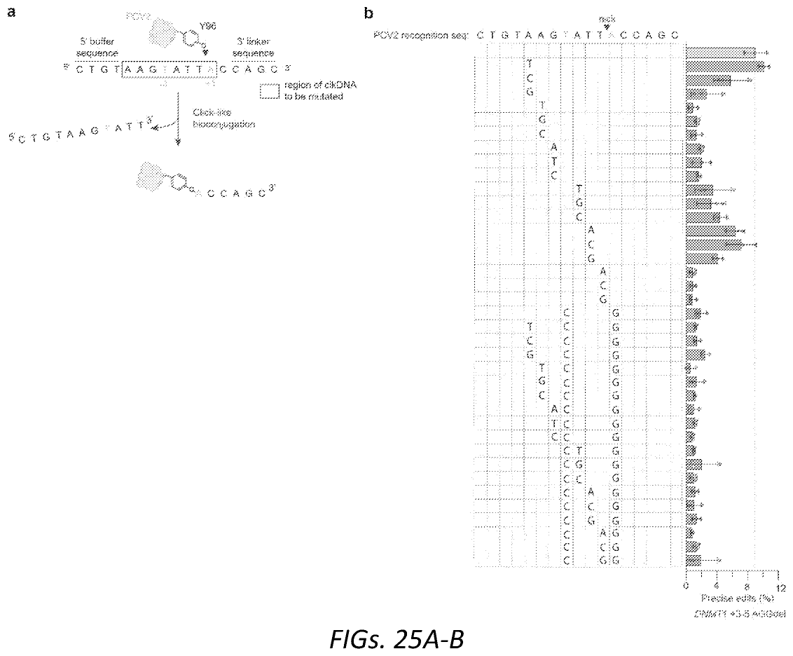

Next, we explored template recruitment modalities to determine the impact on click editing efficiency. Given the diversity of HUHes (FIG. 12B), we tested a variety of domains involved in the replication of circoviruses, geminiviruses and nanoviruses, and the conjugative relaxase TraI³⁵. At both DNMT1 and RNF2, CE1.n2 editing was most efficient with our original CE1 construct containing the PCV2 HUHe fused to nCas9 (FIGS. 1211-J and FIGS. 18A-B). We then tested an expanded catalog of HUHes, including 20 additional orthologs from the circovirus and cyclovirus families⁴⁸ (FIG. 18C). Several HUHe orthologs were similarly efficient to PCV2 for editing at the DNMT1 locus while many were ineffective (FIG. 18D), highlighting that exploration of the diversity of HUHes found in nature can potentiate click editing efficiencies. Next, we investigated telomere binding proteins (TBPs) as a separate effector class that non-covalently binds ssDNA with sequence specificity and high affinity (FIG. 19A). Experiments using wild-type (WT) TBP Cdc13 from S. cerevisiae (Chandra et al., Genes Dev. 2001 Feb. 15; 15(4):404-14) or an engineered Cdc13(Y556A) variant with enhanced binding affinity (Glustrom et al., Proc Natl Acad Sci USA. 2018 Oct. 9; 115(41):10315-10320) in place of PCV2 led to productive click editing, but at lower efficiencies than PCV2-based CEs (FIG. 19 B-C). Finally, we explored whether the PCV2 recognition sequence could be modified to improve editing efficiencies (FIG. 25). We assessed clkDNAs that maintained the −4 and +1 positions as Watson-Crick pairs (Simley et al., mBio. 2023 Feb. 28; 14(1):e0258722) and modified surrounding bases to every other possible base. One variant clkDNA resulted in a modest improvement in editing efficiency (FIG. 25B), indicating that the WT PCV2 sequence may already be near optimal and that further engineering of HUHe binding sites may improve click editing efficiency.

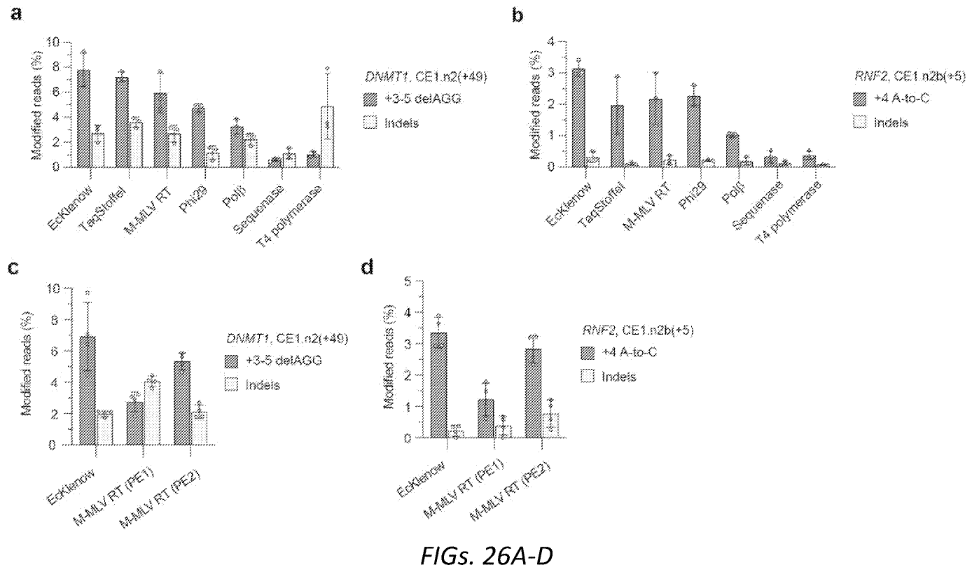

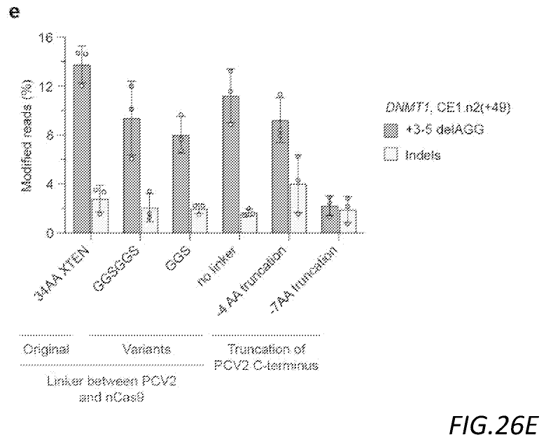

We then explored whether the use of different family A, B, or X DDP enzymes, as well as the engineered reverse transcriptase from the Moloney Murine Leukemia Virus (M-MLV RT) utilized in prime editors (Anzalone, Nature 2019, supra) might alter click editing efficiency. Experiments revealed that at the DNMT1 and RNF2 sites, the Stoffel fragment of Thermus aquaticus DNA polymerase (TaqStoffel⁵²), engineered M-MLV RT (Anzalone, Nature 2019, supra), and bacteriophage Phi29 DNA Polymerase (Phi29 (Blanco et al., J Biol Chem. 1989 May 25; 264(15):8935-40; D169A for exonuclease inactivation (Esteban et al., J Biol Chem. 1994 Dec. 16; 269(50):31946-54) exhibited nearly comparable precise editing to our original CE design containing EcKlenow (FIGS. 12K,L and FIGS. 26A-B). To determine to what extent click editing efficiencies were limited by the use of a WT DDP in the CE, we compared editing efficiencies using WT M-MLV RT (found in the original PE1 construct (Anzalone, Nature 2019, supra) and the engineered M-MLV RT pentamutant (in PE2) which, in the context of prime editing, has been engineered to enhanced binding to the template-PBS complex, enzyme processivity, and thermostability (Anzalone, Nature 2019, supra). Although neither of the M-MLV RT constructs were as efficient as our original EcKlenow-based CE, the engineered M-MLV RT-based CE led to higher editing efficiencies compared to the WT M-MLV RT by 1.9- and 2.3-fold in the DNMT1 and the RNF2 loci, respectively (FIGS. 26C,D). In a separate optimization, decreasing the clkDNA dosage from 16 to 12 pmol increased editing efficiency (FIG. 24D and Example 7), potentially by decreasing gRNA sequestration (from excess clkDNA interactions with the gRNA spacer (Yu et al., Nat Commun. 2022 Dec. 12; 13(1):7545, and Liang et al., Nat Commun. 2022 Jan. 21; 13(1):437) and/or reducing potential cleavage of the gRNA by RNAseH due to the RNA:DNA duplex.

Additional experiments testing alternative linkers between PCV2 and nCas9, or truncations of a flexible C-terminal region of PCV2, did not improve editing (FIG. 26e). Therefore, we proceeded with the PCV2-nCas9-EcKlenow CE construct for further characterization.

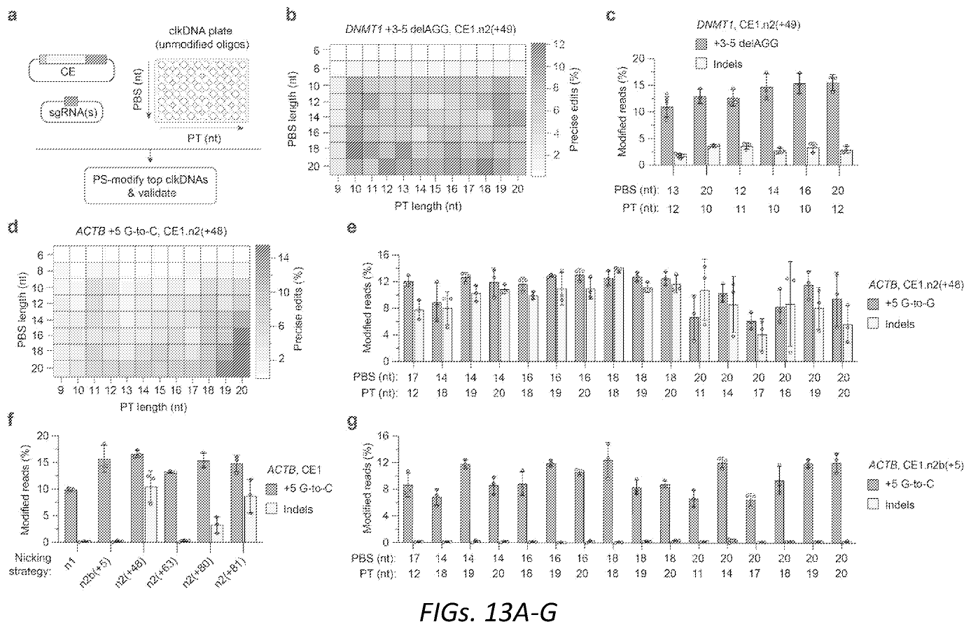

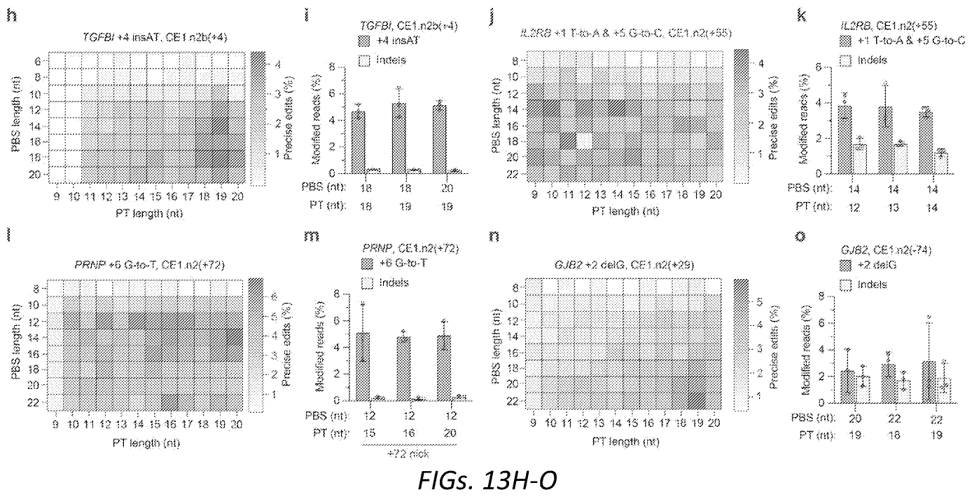

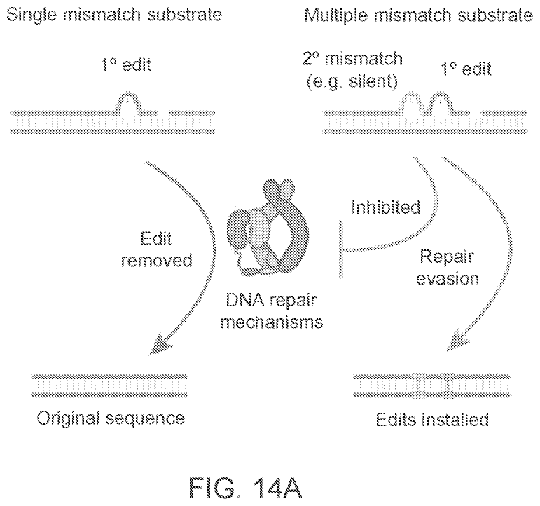

We hypothesized that click-editing efficiency might be improved by testing additional parameters of clkDNA design. For instance, optimal annealing of the PBS to the genomic NTS flap could be crucial to form a stable template-primer junction for polymerase initiation, or the length of the PT could influence flap-genome hybridization and/or flap equilibration (FIG. 12D). The use of HUHes uniquely permits the rapid and high-throughput assessment of clkDNA properties, since HUHes form covalent protein-clkDNA adducts with simple unmodified ssDNA oligos (without requiring chemical or specialized modifications; FIG. 12C). To implement clkDNA optimizations, pre-normalized 96-well plates of simple unmodified DNA oligos can be purchased at relatively low cost, a rapid process that does not require additional cloning steps (FIG. 13A).

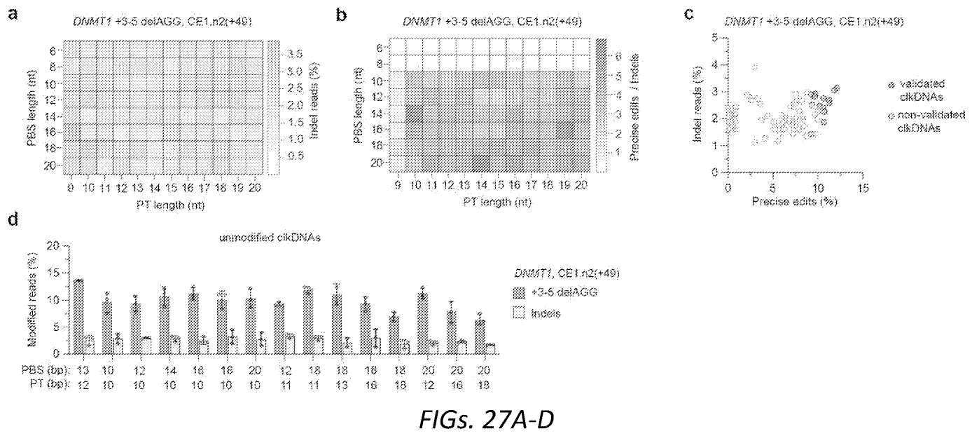

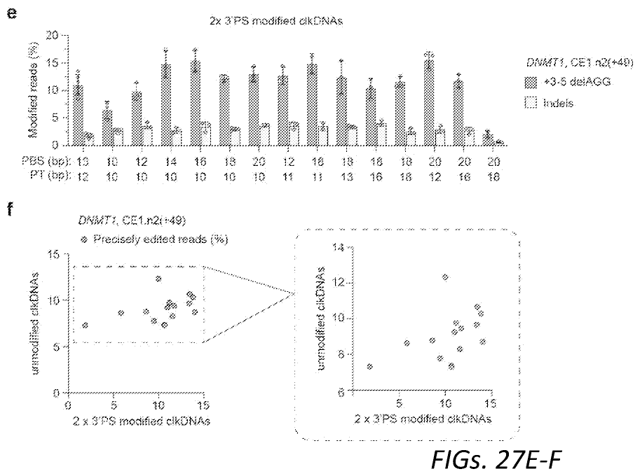

To scalably assess clkDNA parameters, we ordered and screened 96 clkDNA configurations including combinations of PBSs from 6-20 nucleotides (nt) and PTs from 9-20 nt. We initially tested this approach for the DNMT1 +3-5 AGG deletion, which previously yielded nearly 10% precise editing when using a 13 nt PBS and a 12 nt PT clkDNA (PBS13-PT12) (FIG. 12F). Experiments to test all 96 unmodified clkDNA oligos yielded editing with several clkDNAs up to ~12% (FIG. 13B and FIGS. 27A-B). We then performed a validation experiment by selecting 15 clkDNAs that yielded higher efficiencies in the primary screen (FIG. 27C), testing them with two 3′ phosphorothioate (PS) linkages which should improve clkDNA stability (FIG. 27D,E). We observed good correlation between the efficiencies observed with unmodified and PS-modified clkDNAs (FIG. 27F), reaching up to 15.4% precise editing with a PBS16-PT10 modified clkDNA (FIG. 13c) and leading to >50% improvement in efficiency compared to the initial unmodified clkDNA that we had selected (FIG. 12f).

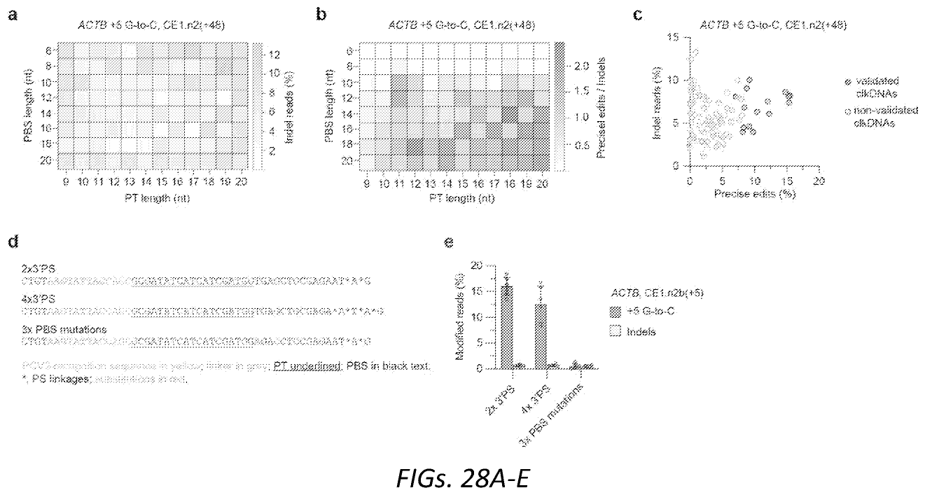

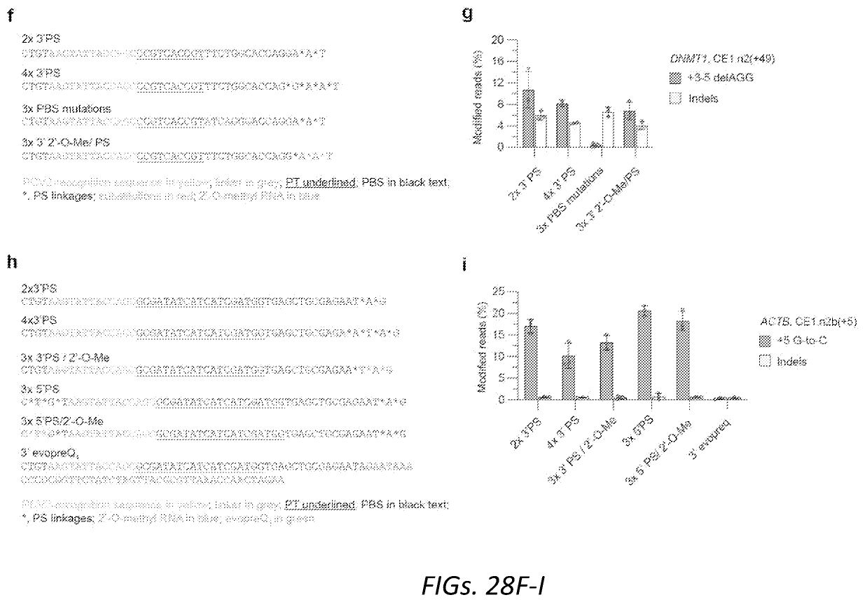

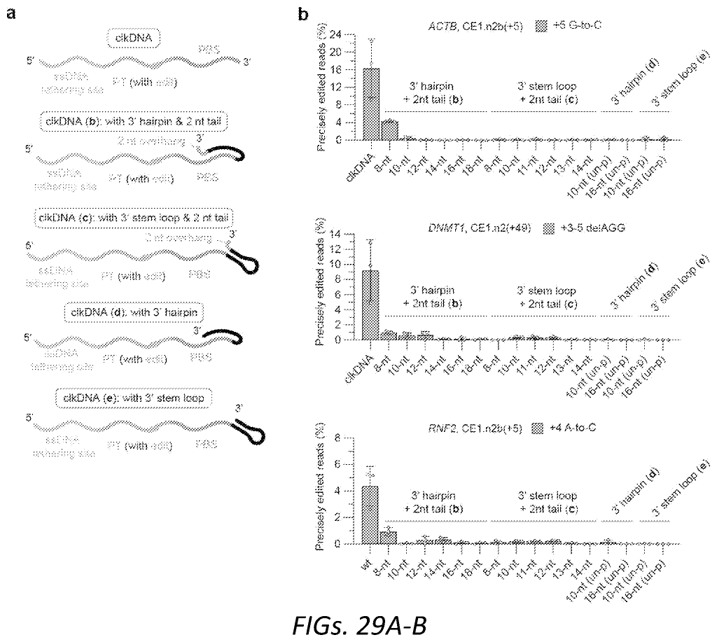

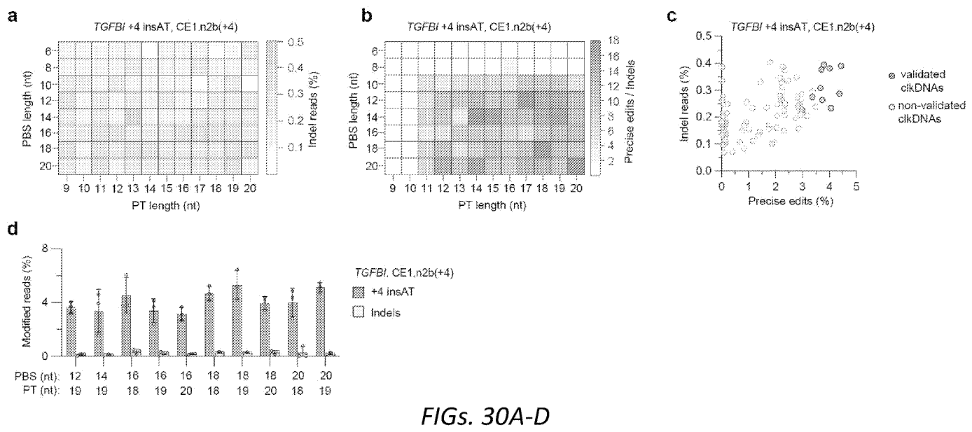

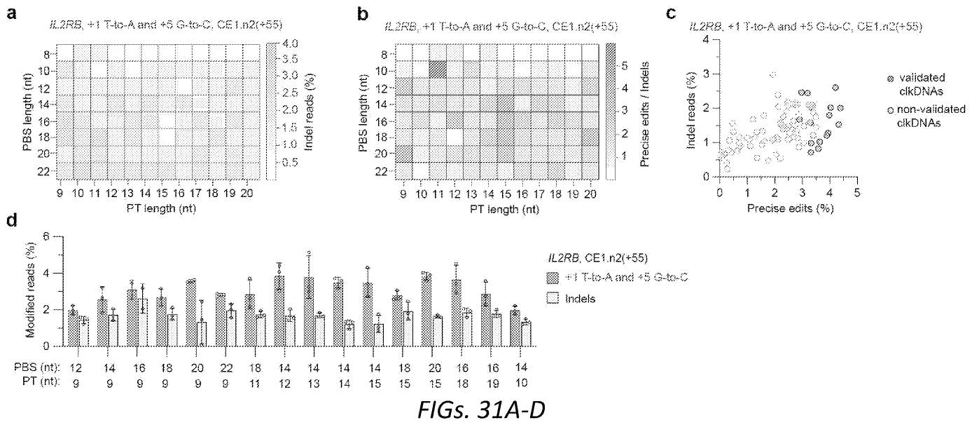

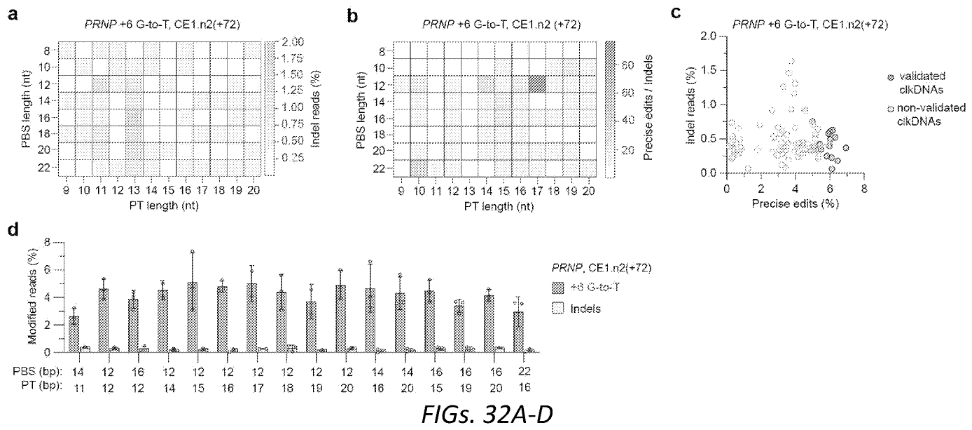

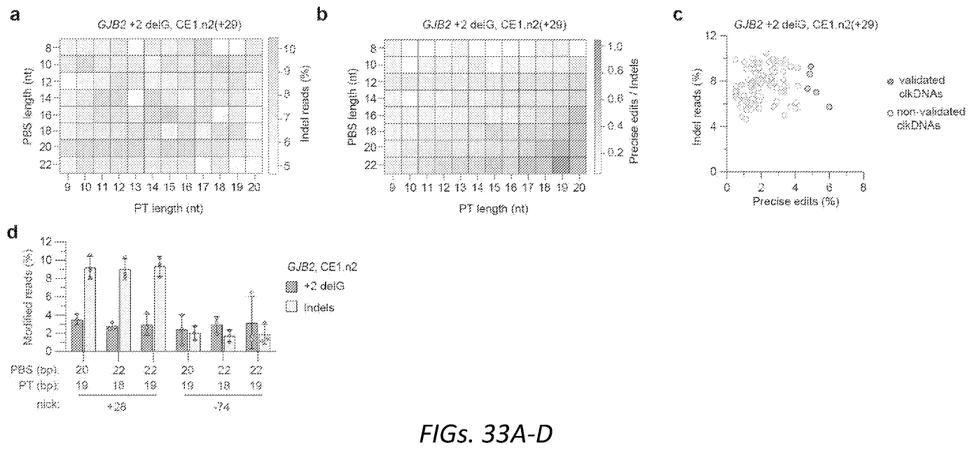

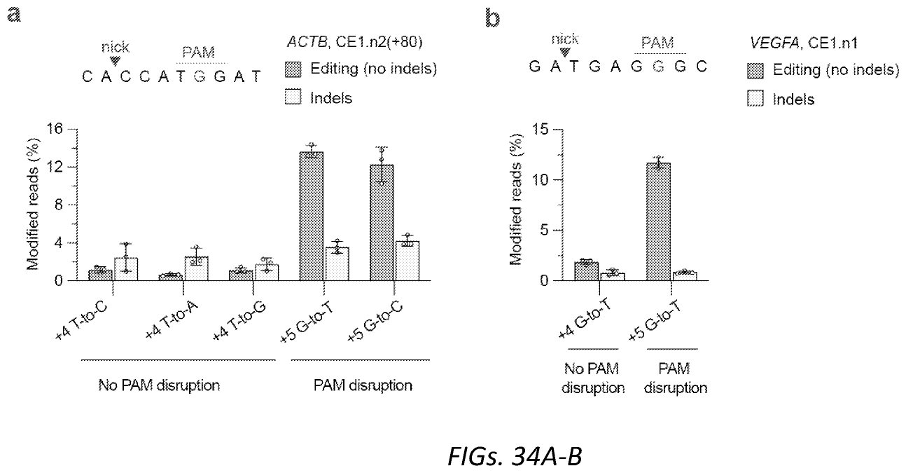

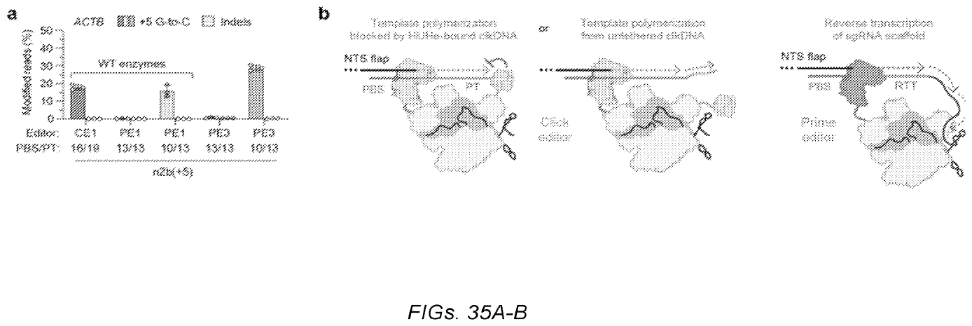

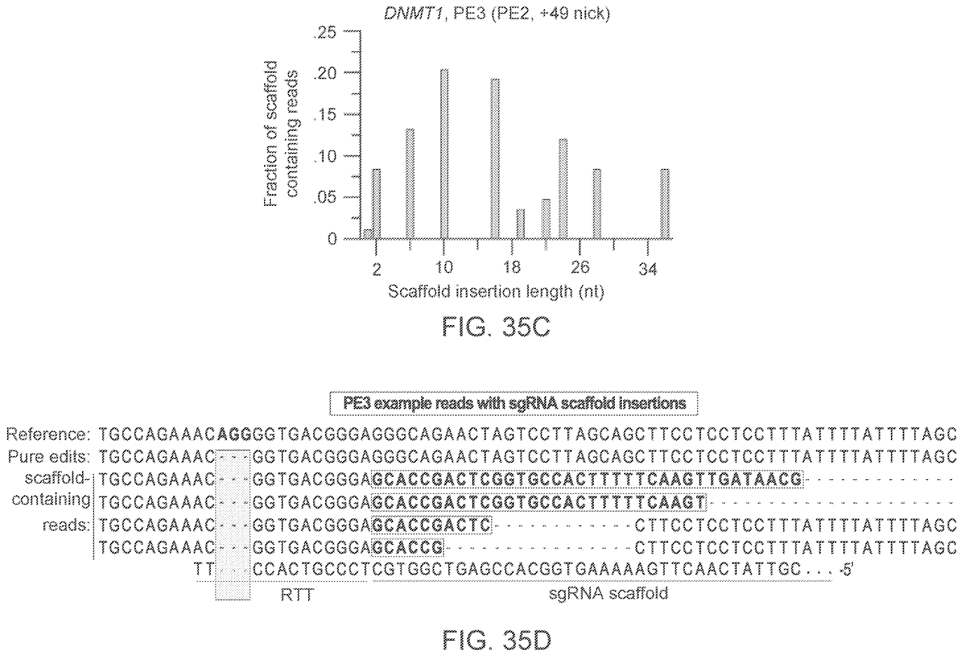

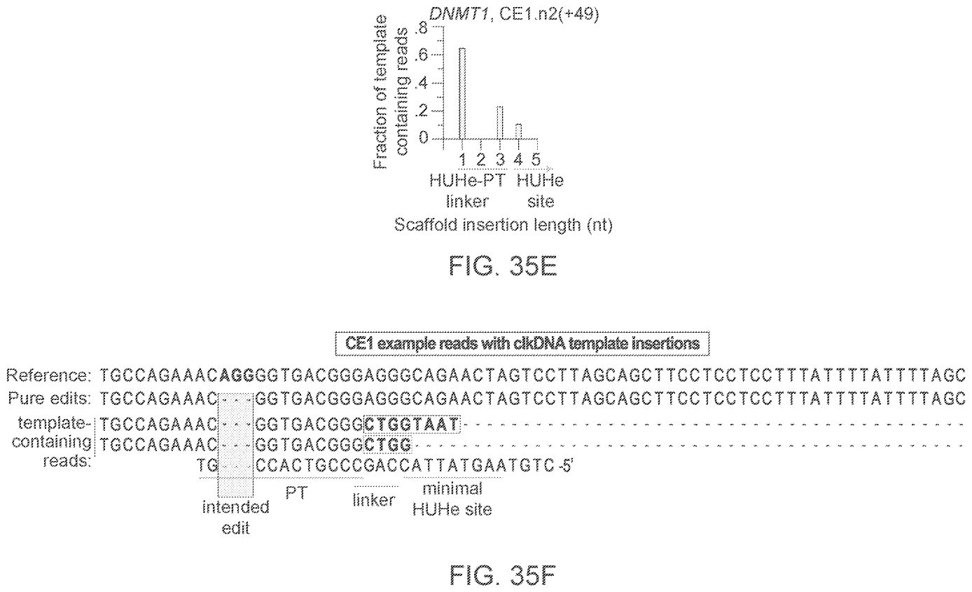

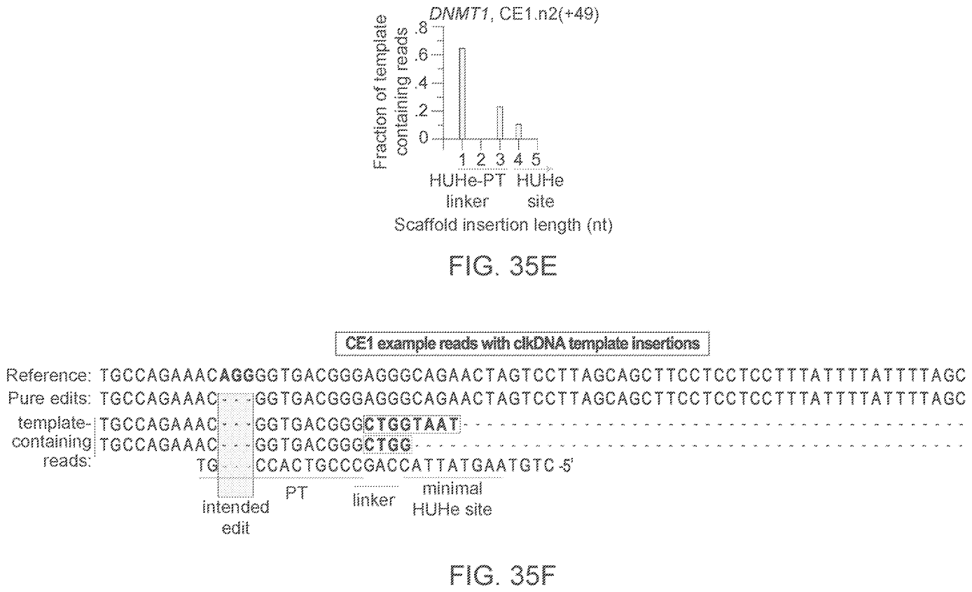

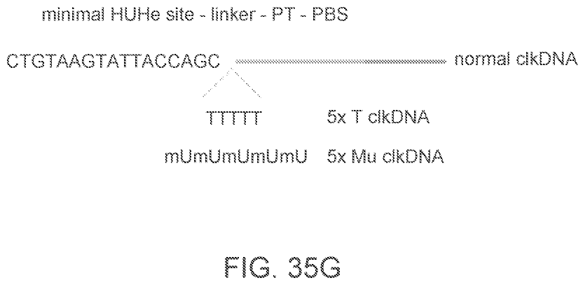

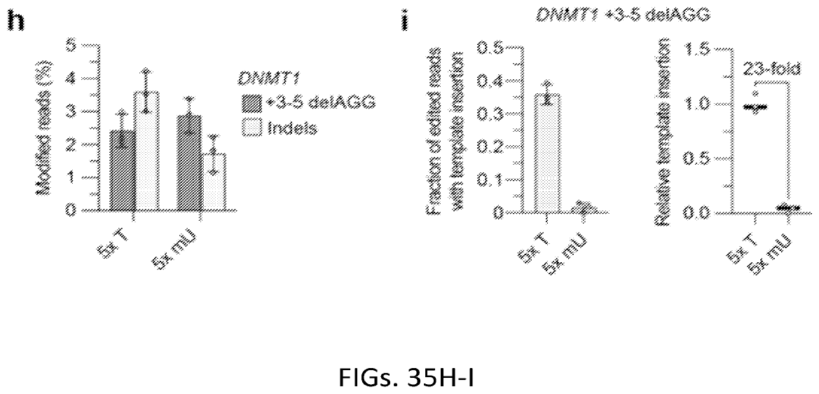

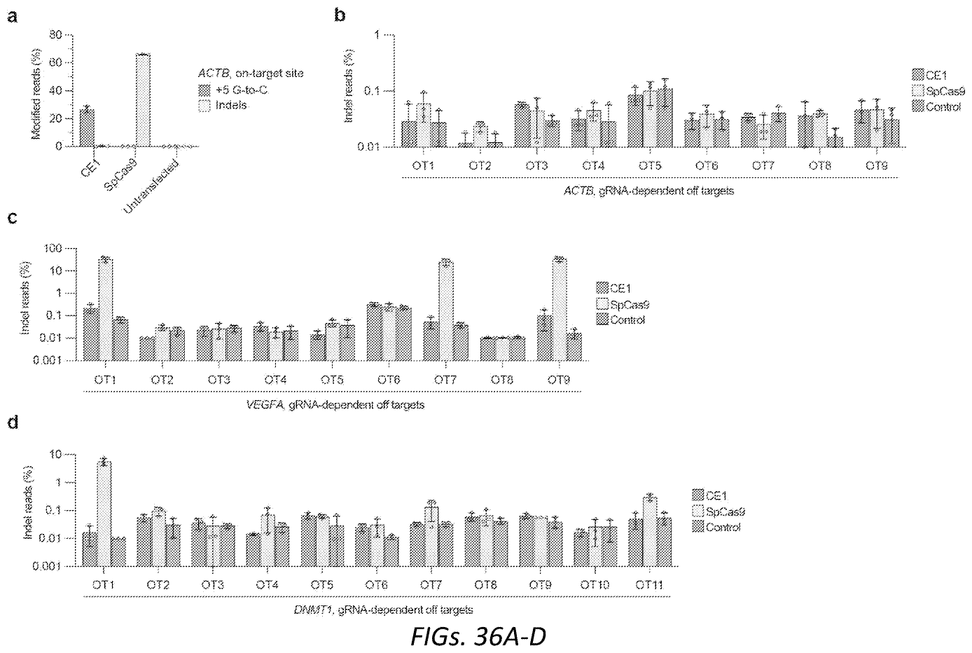

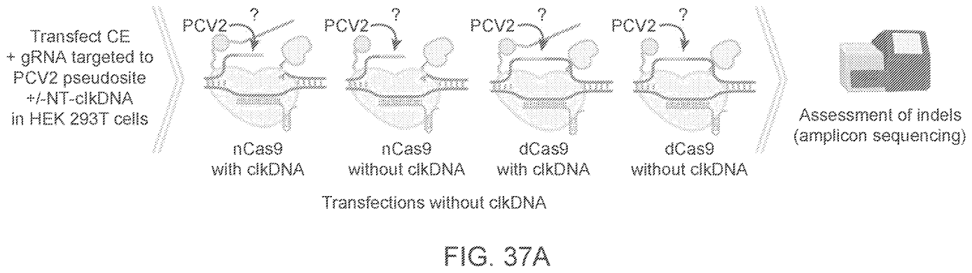

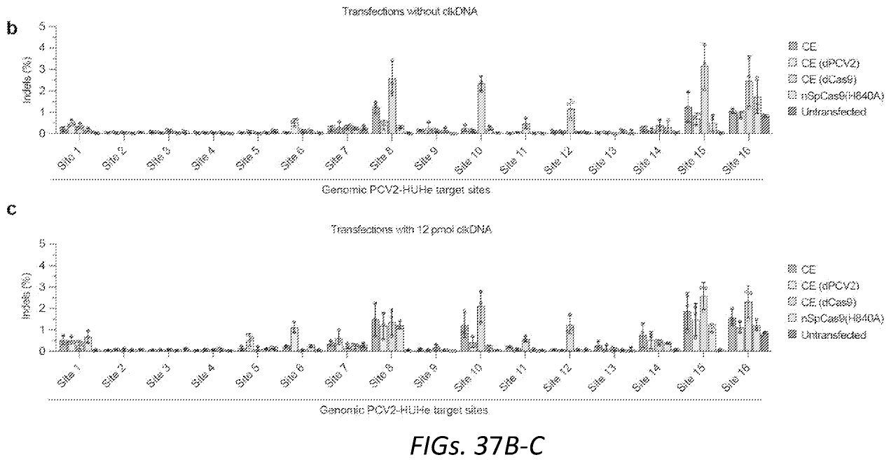

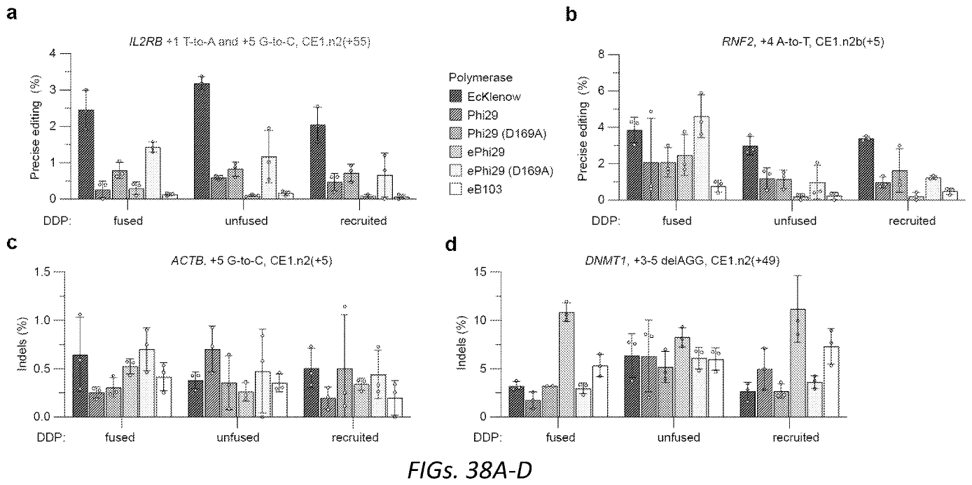

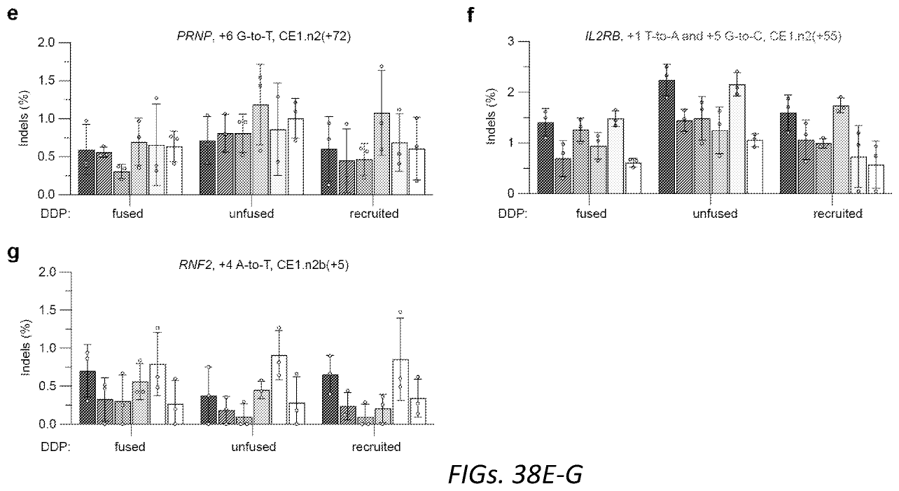

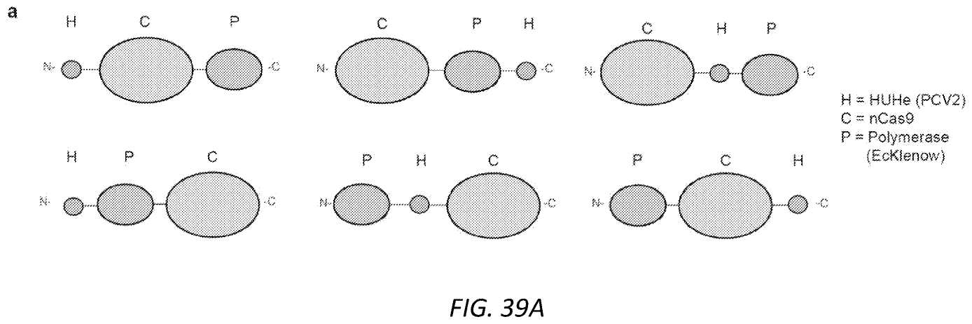

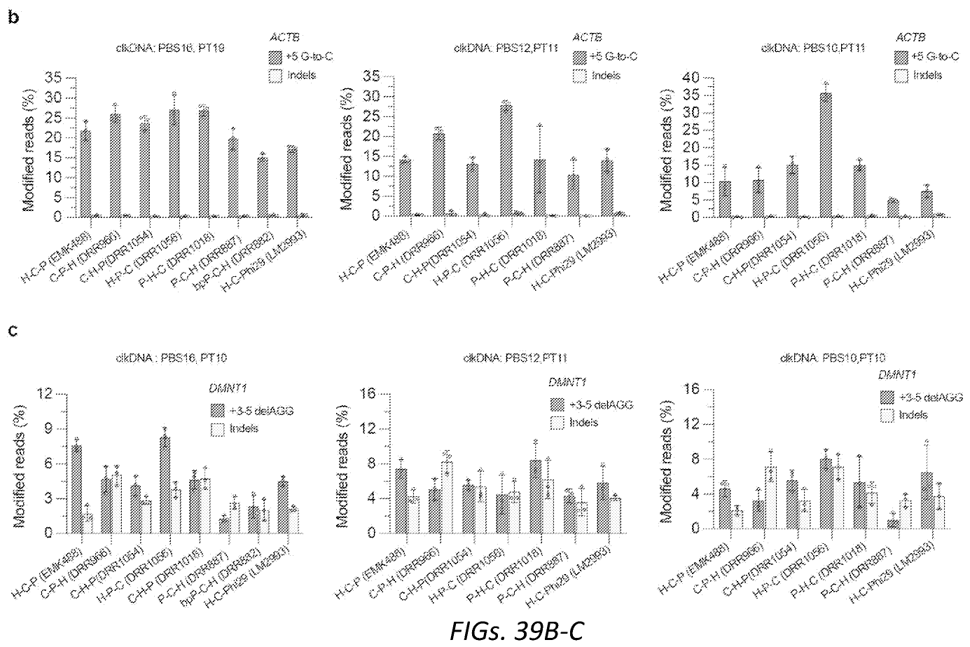

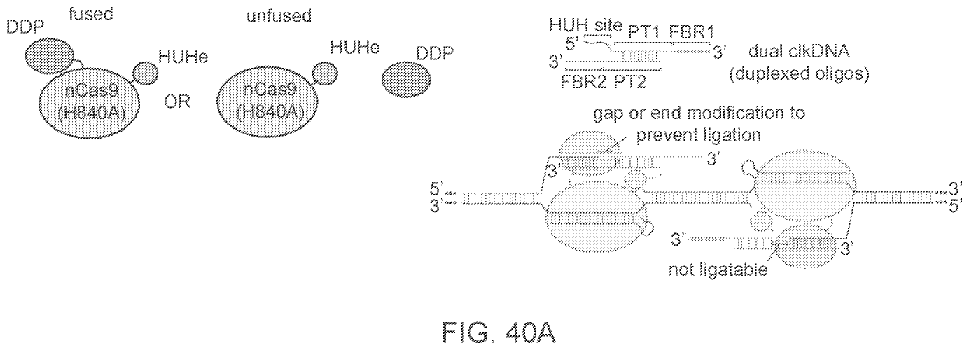

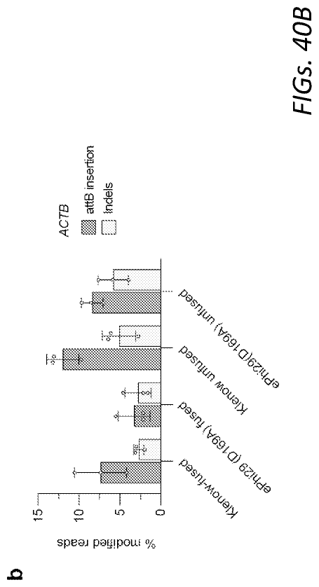

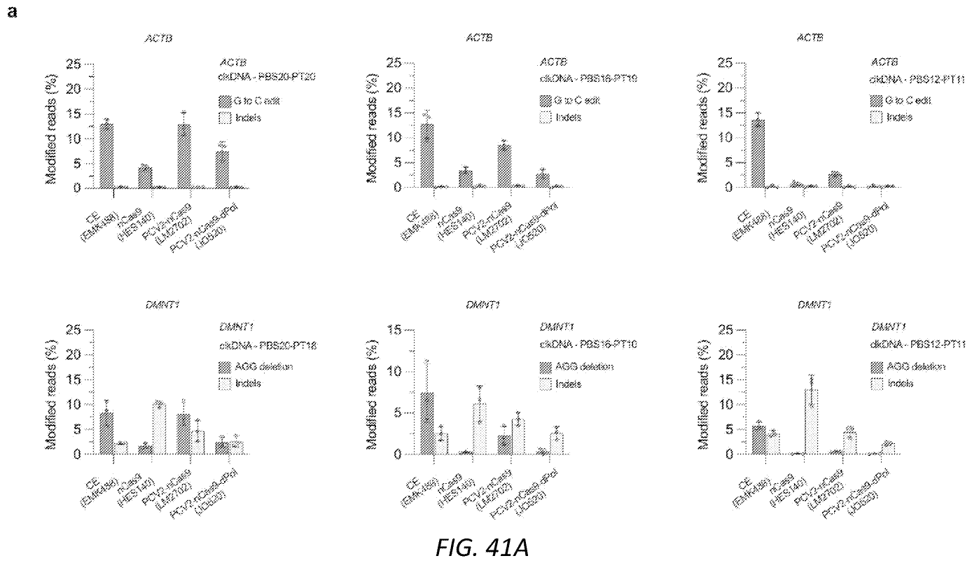

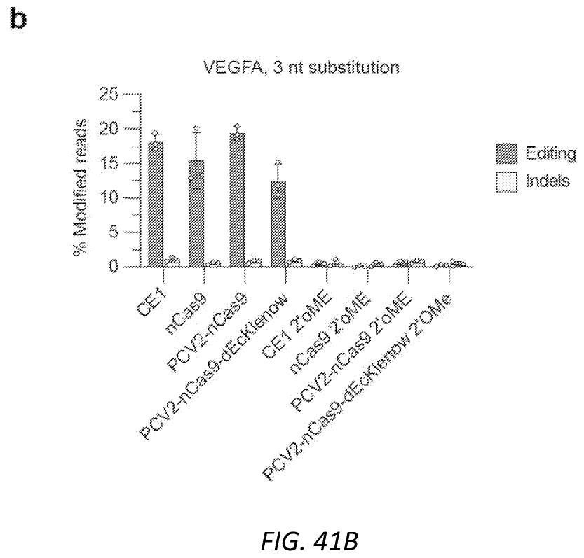

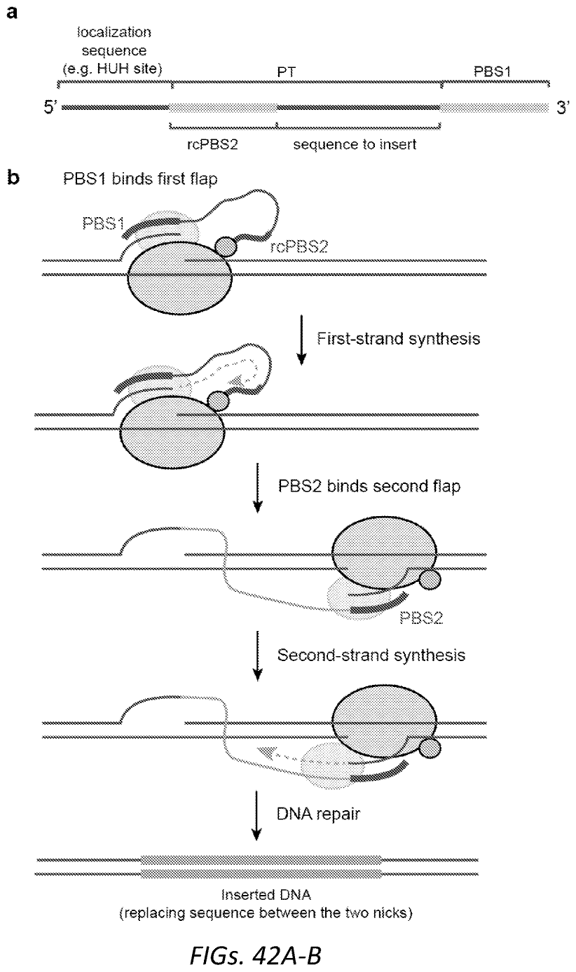

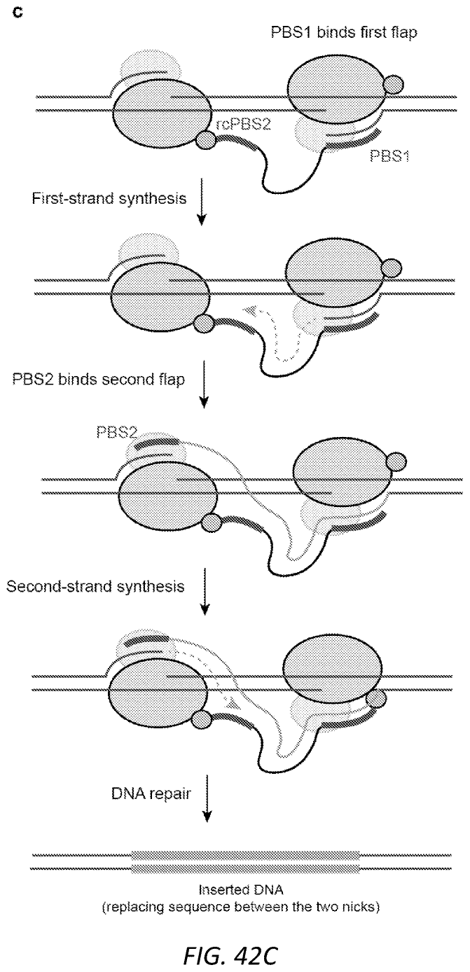

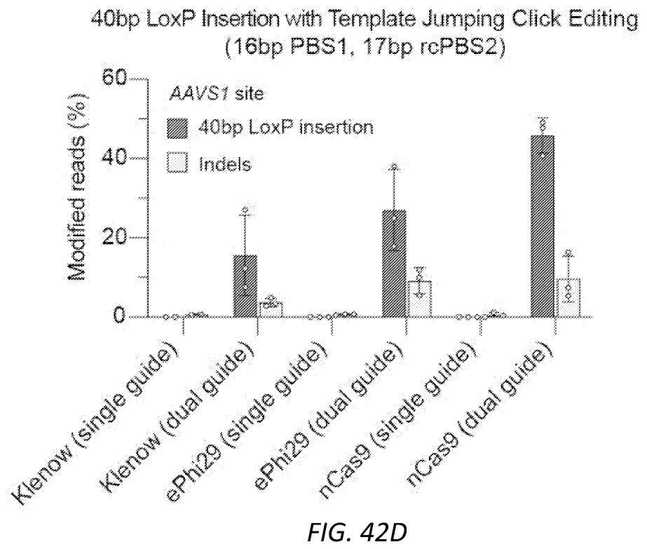

We then explored the generalizability of our scalable clkDNA optimization across other new sites to install various edits. Using an ACTB-targeted gRNA and 96 different clkDNAs encoding a G-to-C transversion, we observed a distinct trend towards higher efficiencies with longer PT and PBS lengths, reaching up to 15.5% precise editing (FIG. 13D). However, we also observed high levels of indels when using the n2(+48) ngRNA (FIG. 2E, FIGS. 28A-C), motivating us to explore additional ngRNAs. Among the CE1 condition with no ngRNA, the +48 ngRNA, and four additional ngRNAs, the n2b(+5) strategy in combination with an optimal clkDNA configuration (PBS16-PT19) yielded 15.74% precise editing with minimal indels (FIG. 13F-G). These results highlight how careful ngRNA and clkDNA selection can dramatically improve edit efficiency and purity.

Next, we tested whether structural modifications or the addition of chemical modifications to the optimal ACTB and DNMT1 clkDNAs (PBS16-PT19 and PBS16-PT10, respectively) could improve editing efficiencies (FIGS. 28D-G). The addition of point mutations within the PBS has been shown to enhance prime editing efficiencies by reducing PBS/spacer complementarity and improving pegRNA folding (Zhang et al., bioRxiv [Preprint]. 2023 Aug. 15:2023.08.14.553324); however, this strategy abrogated click editing, possibly through the reduced affinity between the clkDNA and the NTS genomic flap (FIGS. 28E,G). Similarly, use of a DNA version of a typically RNA evopreQ₁ motif to the 3′ end of the clkDNA, which has been shown to stabilize pegRNAs (Nelson et al., Nat Biotechnol. 2022 March; 40(3):402-410), abrogated click editing in the ACTB locus (FIGS. 28H, I). Interestingly, exploration of different 3′ clkDNA structures largely abrogated editing and induced cell toxicity (FIGS. 29A,B). Separately, we observed that while the addition of 4×3′ PS linkages to the clkDNA reduced editing efficiencies (FIGS. 28 E,G,I), the addition of 2×5′ PS linkages to the clkDNA or 3×5′ PS/2′-O-methyl RNA bases to the clkDNA resulted in a modest increase in click editing efficiencies (FIGS. 28H,I).

We then performed clkDNA screens at additional genomic sites using unmodified clkDNAs. We achieved nearly 5% precise editing at TGFBI for a +4 AT insertion (FIGS. 13H,I, and FIGS. 30A-D), at IL2RB for dual substitutions (FIGS. 13J,K, and FIGS. 31A-D), PRNP for a +6 G-to-T transversion (FIGS. 13L,M, and FIGS. 32A-D), and at GJB2 for a +2 G deletion (FIGS. 13N,O and FIGS. 33A-D). Using over 600 oligos across these six distinct clkDNA screens to install various edits at multiple genomic sites and using several ngRNA types, we observed distinct clkDNA parameter patterns with some common trends, including that PBSs <10 nt do not generally support productive click editing with our current CE configuration, and that longer PBSs and PTs are typically more effective (FIGS. 13B-0). Among these sites and edits, precise click editing using parameter-optimized clkDNAs ranged from 3.50% to 15.74% with minimal indels. Together, the results from our clkDNA optimizations demonstrate that highly precise editing can be achieved through facile clkDNA and ngRNA screening.

Prior studies demonstrated that inhibition of DNA mismatch repair (MMR) can improve prime editing efficiencies by counteracting excision of the edited flap^{76,77}. Design of strategies that install additional edits (e.g. silent mutations) along with desired edit can suppress recognition of the installed mismatch by MMR or other DNA repair mechanisms, thereby increasing editing efficiencies (FIG. 14A). Since the resolution of nascent 3′ DNA flaps encoding mismatches installed by click or prime editing likely proceed through similar repair mechanisms, we investigated whether varying the base composition of the clkDNA PT could enhance MMR evasion to improve precise click editing. We selected a clkDNA to install a dual T-to-A and G-to-C edit at the IL2RB locus, for which we previously achieved nearly 4% precise editing with the PBS14-PT14 clkDNA and CE1.n2 (FIG. 13J). In editing experiments with CE1 and CE1.n2 and various clkDNAs encoding additional silent substitutions, we observed 24.7- and 4.1-fold increases in precise click editing, respectively, with the most effective clkDNA design containing 2 additional silent substitutions, compared to the original design (FIG. 14B). We then designed six clkDNAs for a new edit to install a G-to-T transversion at the VEGFA locus, with various combinations of five encoding additional substitutions for MMR evasion (FIG. 14C). Compared to 12.4% precise editing when using CE1 and a clkDNA encoding only the primary edit, we observed an average of 26.6% editing (with 2.7% indels) using a clkDNA encoding three additional MMR-evading mutations (a 2.2-fold increase; FIG. 14C).

Given the improvement in click editing efficiencies that we observed with MMR-evading mutations for the IL2RB and VEGFA edits, we then took a similar approach for the ACTB edit. However, our initial ACTB clkDNA designs bearing additional mutations did not substantially increase precise editing (FIG. 14D). Considering that the nature of the mutation(s) may bias MMR-engagement, we sought to leverage the scalability of clkDNA synthesis and screening to test a larger and more diverse set of mismatch-harboring clkDNAs. We tested a total of 64 clkDNAs containing all possible combinations of bases in three specific positions within the ACTB clkDNA, along with the +5 G-to-C edit. Interestingly, our screen yielded clkDNAs with diverse impacts on editing efficiency (FIG. 14E) and provided interesting insights into which positions, and types of modifications, within the clkDNA are favorable or detrimental (FIG. 14F). This type of experiment again highlights the scalability of CEs, while also demonstrating how click editing may be utilized to gain insight into biological processes like mismatch repair. Future studies to extend this approach across other loci may provide more generalizable insight into the types and positions of mismatches that maximize edit efficiency.

Beyond the installation of repair evading edits, the precise positioning of the primary or secondary substitutions in the Cas9 target site might impact click editing efficiencies. For instance, the installation of mutations to disrupt the PAM has been shown to improve HDR or prime editing efficiencies (Mathis et al., bioRxiv 2023 https://doi.org/10.1101/2023.10.09.561414; Mathis et al., Nat Biotechnol. 2023 August; 41(8):1151-1159; Paquet et al., Nature. 2016 May 5; 533(7601):125-9. We observed increased editing efficiencies when the substitution was within the guanines of the NGG PAM (FIGS. 34A,B), consistent with prior results that suggest PAM disruption prevents the CE from retargeting the locus after edit installation (Mathis et al., bioRxiv 2023, supra, Mathis et al., Nat Biotechnol 2023, supra; Paquet et al., Nature 2016, supra).

Next, we sought to compare click and prime editing. Since CE1 utilizes an unevolved wild-type EcKlenow polymerase, we included both PE1 and PE2 enzymes in our comparison (which contain a wild-type or an engineered MMLV reverse transcriptase domain, respectively²⁰). The PE2 enzyme combined with an additional ngRNA to direct PE2 nicking is referred to as PE3. In experiments using gRNAs and clkDNAs for CEs, and previously optimized pegRNAs for PEs²⁰, we targeted VEGFA with n1 (no ngRNA), DNMT1 with n2(+49), and ACTB with n2b(+5) nicks. We observed comparable or higher efficiencies with CE1 when compared to PE1, suggesting a similarity in systems when both strategies utilize wild-type polymerases (FIGS. 15A-C). Compared to PE3 at the DNMT1 or ACTB target sites, or to PE2 at VEGFA, CE1 displayed ~2.37-fold lower average precise editing (FIGS. 15A-C), attributed to PE2's engineered polymerase domain. Future efforts to engineer EcKlenow or other DDPs may increase CE efficiency to match or exceed PE2 and PE3 levels.

When comparing CEs and PEs we also analyzed unwanted insertion mutations at the on-target site (FIG. 35B). Since the pegRNA is a fusion of the PBS/RTT with the gRNA scaffold, the RT domain of PEs can install unwanted gRNA scaffold bases into the target site^{10,20,76,78-80} (FIG. 35A). With PE-treated samples, we observed >3% pegRNA scaffold incorporation at the DNMT1 target site but no scaffold incorporation when indels were minimized at ACTB and VEGFA (FIG. 15D and FIGS. 35C,D). Similarly, in our CE condition, we observed that an average of 2.5% of edited reads harbored clkDNA template insertions at DNMT1 but no or minimal incorporation at ACTB and VEGFA (FIG. 15D and FIGS. 35E,F). Further analysis of the CE1 data revealed that most (>70%) template-mediated insertions were derived from a 4 nt linker between the clkDNA PCV2 site and PT (FIG. 35G), which falls outside of the PCV2 protein footprint (Tompkins et al., Nucleic Acids Res. 2021 Jan. 25; 49(2):1046-1064). Only 0.7% of edited reads at DNMT1, and zero reads at ACTB and VEGFA, contained templated byproducts derived only from the PCV2 recognition sequence (FIG. 15D), suggesting that reducing the linker between the clkDNA PCV2 site and PT may minimize undesired base incorporation. Given the HUHe's covalent nature and the fact that most of the HUHe site is cleaved off after binding, we speculate that the minimal incorporation of the consensus HUHe site may be due to free, untethered clkDNA that can bind the genomic flap (FIG. 35B). Because many DDPs are substantially impaired on templates containing consecutive RNA bases (Ricchetti et al., EMBO J. 1993 February; 12(2):387-96; and Krzywkowski et al., Nucleic Acids Res. 2018 Apr. 20; 46(7):3625-3632), we also sought to minimize template byproducts via the use of modified RNA bases between the HUHe site and the PT. clkDNAs harboring of 5×5-methoxyuridine (mU) between the HUHe site and the PT decreased HUHe incorporation by 23-fold compared to an analogous clkDNA harboring 5×T bases (FIGS. 35G-I). Together, CEs maintain highly pure editing outcomes, and various strategies including clkDNA sequence optimization and clkDNA modifications may minimize or eliminate template incorporation.

Like the high fidelity mechanism of prime editing⁸²⁻⁸⁵, click editing also requires several proof-reading steps that may reduce the likelihood of gRNA-dependent off-target editing, including pairing of the gRNA spacer with the genomic target site, clkDNA PBS annealing to the NTS, annealing of the nascent 3′ flap to the genomic locus for edit resolution, along with use of a nickase rather than a nuclease. To investigate potential off-target edits when using CEs, we targeted a CE or Cas9 nuclease to three target sites in the VEGFA, ACTB, and DNMT1 loci. We simultaneously assessed on-target editing and potential off-target editing at putative off-target sites closely related in sequence to the on-target sites²⁴ (see Methods; FIGS. 15e-i and FIGS. 36A-D). Across 29 off-target sites, when using SpCas9 nuclease we observed considerable indels at three VEGFA off-target sites (33.9%, 25.6%, 34.2%) and two DNMT1 off-target sites (5.7%, 0.3%) (FIGS. 15f,h, and FIGS. 36B-D). With CEs, we observed dramatically lower off-target indels near or below the level of detection, as determined by untransfected samples. We observed no detectable precise off-target edit installation, supporting a potential high-fidelity mechanism of click editing (FIGS. 15F,H, and FIGS. 36B-D).

Since HUHes are DNA endonucleases, we also investigated whether spurious interaction of PCV2 HUHe with genomic ssDNA could result in undesired indels at endogenous PCV2 pseudosites (FIG. 15J). We identified 16 PCV2 consensus sites in the human genome near NGG protospacer-adjacent motifs (PAMs) (Example 9). Using CE-treated samples containing DNMT1-, RNF2, or ACTB-targeted gRNAs and clkDNAs (resulting in efficient click editing at the intended on-target sites; FIG. 15K), we amplified and sequenced four PCV2 genomic sites, which revealed no elevation in indels compared to untreated control samples (FIG. 5L). When we intentionally induced stable R-loops and accessible ssDNA at the PCV target sites in a reporter assay (using the CE construct and gRNAs targeted to the PCV sites; FIG. 37A), we observed slightly elevated HUH-mediated indels at these four sites (FIGS. 37B-E and Example 9). Together, these results demonstrate that CE-fused HUHe enzymes are specific towards the HUHe ssDNA recognition sequence encoded on the clkDNA and carry little risk of genomic off-targets, but also suggest that rare occurrences of gRNAs with spacers matching the HUHe sequence should be avoided.

Lastly, we performed a CE architecture optimization to determine the importance of DDP fusion to nCas9, and that also included alternative DDPs from Phi29 and Phi29-like phages given their inherently high fidelity and processivity. While we previously observed slightly lower click editing efficiencies in our initial CE constructs when testing Phi29 DDP compared to EcKlenow CEs (FIGS. 12K,L, and FIGS. 26A-B), we recognized that our previous N-terminal fusion could be detrimental to polymerization activity⁸⁶⁻⁸⁸ and also that Phi29 is optimally active at 30° C. rather than 37° C.^{86,87}. To explore whether Phi29 or similar polymerases could support click editing, we tested wild-type Phi29, an engineered thermostable Phi29 (ePhi29)⁸⁹, and an engineered thermostable Phi29 ortholog (eB103)⁹⁰ in fused, unfused, or polymerase recruited CE architectures (the latter via N5/N6 coiled-coil domains^{21,22}) (FIG. 15M). Analysis of precise editing at DNMT1, ACTB, PRNP, IL2RB and RNF2 revealed optimal activities with our original EcKlenow polymerase, demonstrating comparable efficiencies across each of the three nCas9-DDP configurations (FIGS. 15N-P and FIGS. 38A-B). Phi29 could also support click editing, with generally increased efficiencies in the unfused configuration and with exonuclease inactivation (D169A). The previously engineered thermostable ePhi29 or B103 mutants did not lead to increased editing efficiencies. Indels varied across configurations, with generally higher rates in the unfused configurations and when using ePhi29 (FIGS. 38C-38G). With the exception of RNF2, these comparisons were performed using clkDNAs resulting from our screens that have been optimized for EcKlenow activity; it's therefore possible that alternative polymerases may differ in optimal clkDNA parameters. Together, these results demonstrate that the modularity of the CE complex can enable productive click editing with various polymerases and architectures.

### Example 7. MSA-Biotin and MCP-MS2 clkDNA Recruitment

Recently, a complementary study also harnessed the advantageous properties of DDPs to create DNA polymerization editors (DPEs) (Liu et al., Nat Biotechnol. 2023 Sep. 14. doi: 10.1038/s41587-023-01947-w). DPEs consist of Phi29 polymerase fused to a template a recruitment module (monomeric streptavidin, mSA, or MS2 coat protein, MCP), which is co-expressed with a separate nCas9(H840A) construct. We therefore tested CEs comprised of either mSA-nCas9-DDP or mSA-DDP+nCas9 architectures with EcKlenow or ePhi29, along with 5′ biotin labeled substrates (FIG. 20A). In experiments comparing HUHe to biotin-mediated template interaction, we observed that while click editing works efficiently in the configuration with PCV2-DDP (EcKlenow and ePhi29) and nCas9 expressed separately, replacing PCV2 by mSA led to generally lower editing efficiencies when editing the ACTB or DNMT1 loci (FIG. 20B,C).

Additionally, we tested CEs that utilize MCP as the template recruitment module for the clkDNA (which encodes an MS2 stem-loop (Tutucci et al., Nat Methods. 2018 January; 15(1):81-89.), and also tested CEs that are comprised of EcKlenow, Phi29, or ePhi29 DNA polymerases (FIG. 21A). Two distinct configurations for the MS2-encoding clkDNA were tested, where the 5′-end either did or did not include three 2′-O-Methyl RNA bases. The results demonstrated that MCP-MS2 recruitment of the clkDNA is highly efficient and enables precise click editing, reaching up to 50% precise editing efficiency in the ACTB locus and up to 20% in the DNMT1 locus (FIG. 21B). No substantial differences were observed in editing efficiencies obtained when using a CE expressing a wild-type or engineered MCP (N55K) (FIG. 21C). This observation might be explained by the usage of an engineered MS2 stem-loop which already possesses increased affinity to MCP (tenfold higher when compared with the wild-type MS2 stem-loop) (Tutucci et al., Nat Methods 2018, supra). Next, we tested the MCP-MS2 CE system in an unfused architecture (MCP-DDP+nCas9) (FIG. 21D). Our results showed that this architecture was compatible with productive editing, achieving approximately 40% precise editing in the ACTB locus and 13% in the DNMT1 locus (FIG. 21E).

### Example 8. Testing CEs with Other Cas9 Orthologs

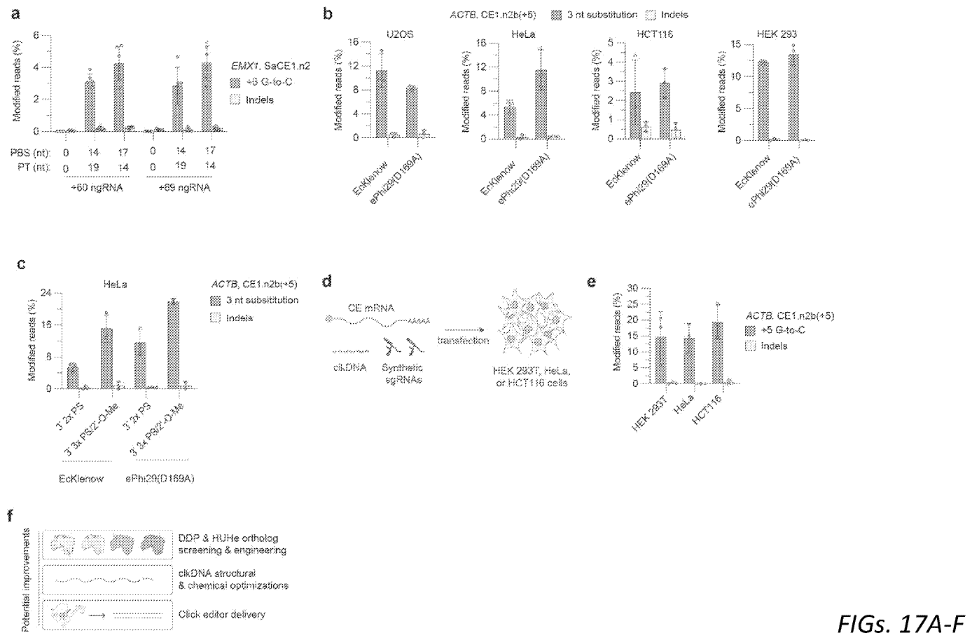

We wondered whether CEs were functional with other Cas9 orthologs. We generated a CE comprised of the Staphylococcus aureus Cas9 (PCV2-nSaCas9(N580A)-EcKlenow). Experiments using SaCas9-CE1, two different n2 ngRNAs, and a clkDNA harboring a +5 G-to-C substitution in the EMX1 locus led to click editing efficiencies ranging from 3-4% precise editing, depending on the clkDNA and ngRNA used (FIG. 17A). These results demonstrate the compatibility of CEs with other DNA-targeting domains, like SaCas9.

### Example 9. Extensibility of CEs to Other Cell Systems

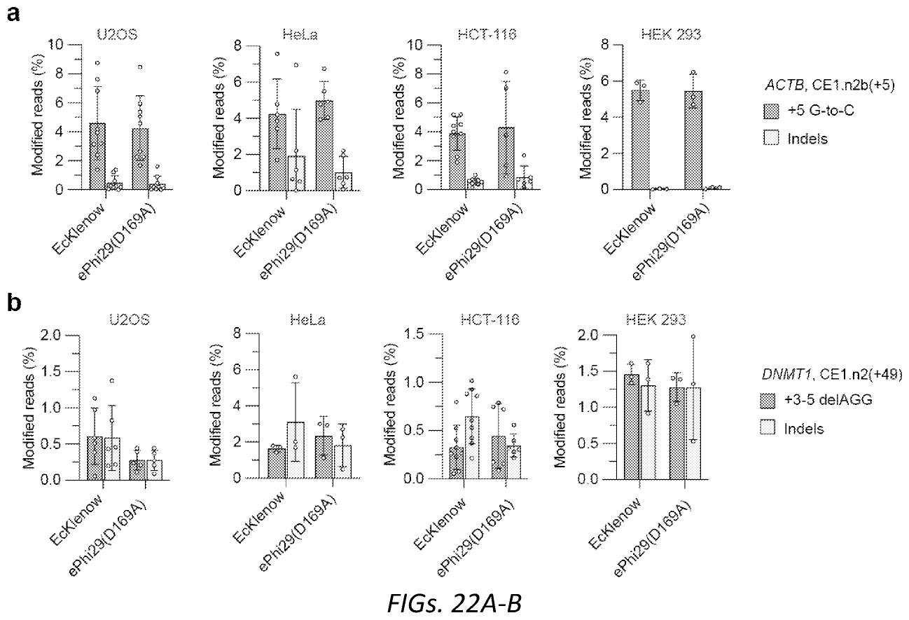

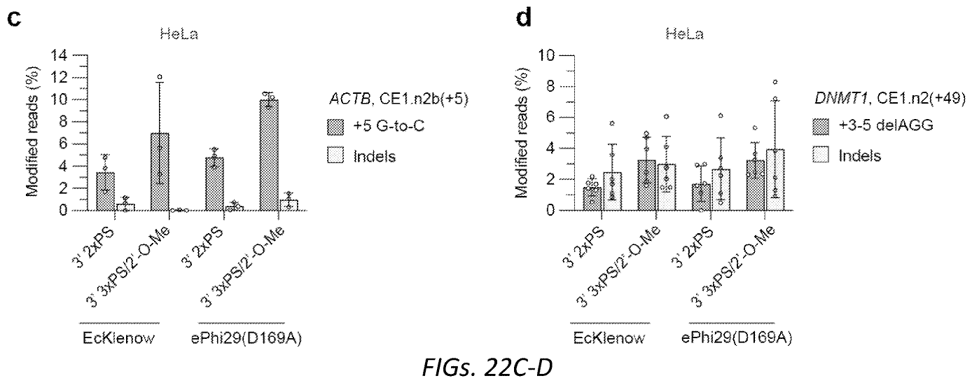

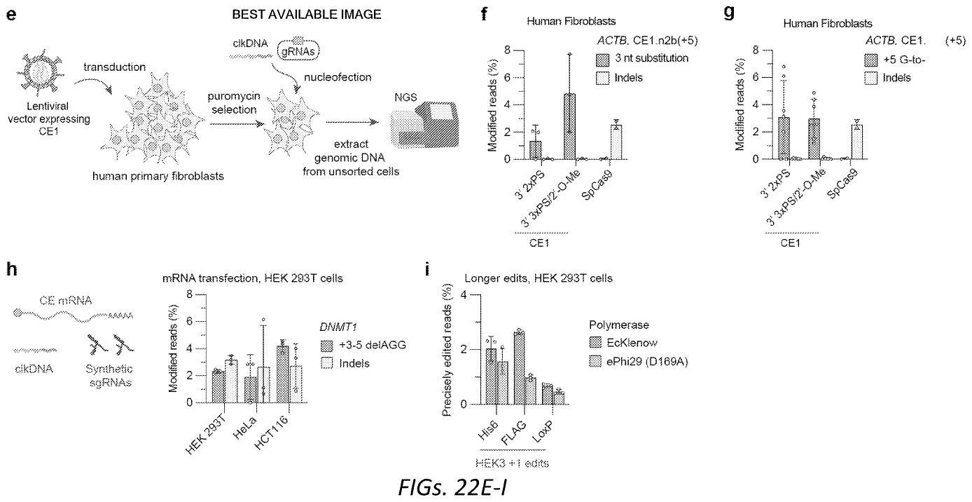

Finally, we tested the extensibility of click editing across other human cell lines and edit types. Using human U2OS cells (bone osteosarcoma epithelium), HeLa cells (cervical cancer), HCT-116 cells (colorectal cancer), HEK 293 cells (embryonic kidney; this cell line does not express the T antigen from SV40 and is mismatch repair proficient⁸⁶), we installed three different edits across the ACTB and DNMT1 loci. Click editing efficiencies in these cell lines ranged from 0.32-13.5%, depending on the edit and the DDP used (FIG. 17B and FIGS. 22A,B). Moreover, the use of clkDNAs with 3×3′ PS/2′-O-methyl RNA bases led to a 1.89-to-2.8-fold improvement in editing efficiencies in HeLa cells, for up to 21.9% precise editing (FIG. 17C and FIGS. 22C,D). Click editing in primary human fibroblasts stably expressing the CE1 construct (FIG. 22E) resulted in up to 7-8% editing efficiencies for two different edits in the ACTB locus using unsorted and unselected cells (FIGS. 22F,G). Furthermore, delivery of CE mRNA, synthetic sgRNAs, and clkDNAs in HEK 293T, HeLa, and HCT-116 cells resulted in productive click editing at both ACTB and DNMT1 loci up to 25.2% and 4.6%, respectively (FIG. 17D,E and FIG. 22H). To test the ability of CEs to mediate longer and more complex edits, we tested clkDNAs to install various sequences (18-40 bp) at the HEK3 target site and observed evidence of insertion (FIG. 22I). Overall, these results demonstrate the applicability of CEs to other cell models, various delivery modalities, and the feasibility of installing larger insertions.

### Example 10. Optimization of clkDNA Dose

To determine the optimal dose of clkDNA to use for click editing experiments, we co-transfected HEK 293T cells with CE1 (PCV2-nSpCas9(H840A)-EcKlenow), a primary gRNA targeting DNMT1, and a secondary ngRNA (n2(+49)), together with increasing doses of a clkDNA (from 0-32 pmols) encoding a +3-5 AGG deletion (FIG. 24C). In a control condition, we co-transfected an nSpCas9(H840A) with both gRNAs and a similar clkDNA lacking the PCV2 recognition site (i.e. only PT and PBS) (FIG. 24C). Our results confirmed that productive click editing only occurs in the CE condition. Moreover, the optimal clkDNA dose for this experiment ranged between 10 and 16 pmols which, considering the length and composition of this particular oligonucleotide, is equivalent to 128.8-206.1 ng of ssDNA. The highest dose tested (32 pmols) induced some toxicity to the cells, which might explain the decrease in click editing efficiency. Considering these results, for a selection of subsequent experiments (FIG. 12 F-L; FIGS. 18 A-B and FIGS. 26A and B) we used 16 pmols of clkDNA and 0.6 μL per 100 ng of total DNA of transfection reagent (TransIT-X2, Mirus). However, upon repeating transfections several times, we noticed that in some cases these conditions could induce toxicity, a potential cofounding factor for accurately determining editing efficiencies. We also hypothesized that high doses of clkDNA may decrease editing efficiencies through spacer sequestration (due to sequence complementarity⁷⁵), or RNaseH-mediated degradation of DNA:RNA hybrid created by pairing in solution between a clkDNA and the spacer of a gRNA. We then performed an additional test to determine whether decreasing the clkDNA dose to 12 pmols would improve editing and viability (FIG. 24D). In this experiment, we also decreased the concentration of transfection reagent to 0.5 μL per 100 ng of total DNA. These new conditions led to a 1.37-fold increase in click editing efficiency compared to the previous experimental setup with no alteration in indel levels (FIG. 24B), and significantly less general toxicity to cells. All experiments for the remainder of our study were performed with these optimized conditions (12 pmols of clkDNA and 0.5 μL of TransIT-x2 per 100 ng of total transfected DNA). We envision that further optimization of clkDNA chemistry, as well as alternative delivery modalities (e.g. RNPs), will permit a reduction of clkDNA dosage to alleviate any clkDNA:spacer sequestration of RNaseH-mediated gRNA cleavage without compromising editing efficiencies.

### Example 11. Analysis of Click Editor Controls and Indels

We achieved productive and precise click editing when using our original CE construct (PCV2-nCas9(H840A)-EcKlenow; FIGS. 12E-G). To confirm that all components were essential for productive click editing, we performed experiments using a series of control conditions that included: (1) a nickase Cas9 (H840A) (nCas9) with a clkDNA that did not contain an HUH site (i.e. PBS/PT only); (2) a CE containing a dead Cas9 (H840A, D10A) but a functional PCV2 and EcKlenow; (3) a CE containing a dead PCV2 (Y96F) (dPCV2)³⁶ but a fully functional nCas9 and EcKlenow; and (4) a CE containing an attenuated EcKlenow (D355A, D357A) (dKlenow)^{91,92} but a fully functional nCas9 and PCV2 (FIGS. 12F,G), and clkNAs shown in Table 4. Although our control conditions confirmed the essentiality of all active components for productive click editing, we could still detect low level precise editing when using a CE containing a dPCV2 or dKlenow. For click editing with a dPCV2-CE, it is possible that the catalytically inactivated PCV2 domain may retain some residual DNA binding affinity (but be unable to catalyze covalent adduct formation with a clkDNA), enabling weak localization of the clkDNA to the target site-bound dPCV2-CE (which contains a fused, fully active EcKlenow). Alternatively, the PBS from a clkDNA from solution could hybridize with the nCas9-induced NTS flap, providing weak but sufficient annealing of the clkDNA:NTS duplex to initiate polymerization. When testing the CE construct fused to dKlenow, a clkDNA should be covalently localized to the target site via PCV2 but polymerization should be substantially attenuated from the fused polymerase⁹² (or any other CE-fused polymerase in trans). In this case, the low levels of precise click editing that we observed may be the result of incomplete inactivation or EcKlenow, or endogenous polymerases interacting with the clkDNA-NTS hybrid to initiate polymerization.

In this experiment we also compared CE1 with CE1.n2 and CE1.n2b (FIGS. 12F,G). While CE1.n2 employs a secondary gRNA to direct CE-mediated nicking (ngRNA) against the non-edited strand at a certain distance upstream or downstream of the primary nick site, CE1.n2b uses a ngRNA to nick the non-edited strand only after the edit is installed (FIG. 23). Because the CE1.n2b strategy minimizes concurrent nicks on opposite strands, the probability of the reaction resulting in insertion or deletion mutations (indels) is reduced (similarly to as observed with PE3 and PE3b nicking events for prime editing²⁰) (FIG. 12G).

### Example 12: Expanded Discussion of HUHe Off-Target Analysis

Aside from their use to tether ssDNA templates as homology-directed repair donors to Cas9-induced sites of DNA breaks³⁷, the uses of HUHes have been largely unexplored in the context of genome editing experiments. We sought to better understand any potential impacts of overpressing an HUHe in human cells, which we imagined might be relatively innocuous given HUHes dual modes of specificity: (1) being specific for ssDNA (and not dsDNA) and (2) requiring a specific DNA binding motif on the substrate³⁴. While the vast majority of the human genome is double-stranded in most cell types, regions of ssDNA can become transiently exposed during transcription and/or DNA replication, including genomic hotspots that are prone to ssDNA deamination events from endogenous deaminases⁹³⁻⁹⁵. Fortunately, RPA, hSSB, and other process-specific factors can occupy these regions to limit access or damage to ssDNA. Moreover, these ssDNA regions tend to be transient; thus, sustained solvent exposure of a specific region of ssDNA containing the full HUHe recognition sequence is unlikely. Thus, we wondered whether intentionally overexpressed HUHes could act on perfectly or partially matched HUHe binding pseudosites, which may exist in the human genome during click editing.

In an initial experiment, we identified genomic sites bearing a perfectly matched PCV2 HUHe binding sequence (AAGTATTACCAGC (SEQ ID NO: 210)) using TagScan⁶⁶ (Table 5). After constitutively overexpressing a CE or HUHe domain in cells for ~72 hours, we extracted genomic DNA and sequenced these regions to determine whether we observed an increase in indels in HUHe-treated conditions. Despite performing our experiments in rapidly dividing and highly transcriptionally active HEK 293T cells, we were unable to detect indels at these genomic HUHe sites at levels above untransfected controls (FIGS. 15J-L and FIGS. 36D,E).

To improve our sensitivity to detect potential HUHe-genomic interactions, we searched for and nominated additional PCV2-HUHe sites within 20 bp of an NGG PAM, optimally placing the PCV2 binding site in the solvent-accessible PAM-distal region of the non-target strand (to improve the sensitivity of potentially detecting HUHe-based indels by artificially maximizing access of the CE or HUHe to the ssDNA NTS).

To test this possibility, we performed a reporter experiment where we intentionally targeted the CE (with nCas9 or dCas9) to HUHe genomic sites to induce an R-loop (FIG. 37A). By creating contrived stable R-loops, we were able to detect low level indels at levels slightly greater than control conditions (FIGS. 37B,C). We observed indels only when the HUHe pseudosite was in a very specific region of the Cas9 target site, an effect that was dampened slightly by the addition of a clkDNA in the transfections. We anticipate that stable R-loops at these very sparse/rare genomic locations containing an HUHe recognition sequences should occur infrequently if at all, though future studies are required to more thoroughly interrogate the genome-scale impact of HUHe overexpression.

### Example 13: Testing Various Click Editor Architectures

Our initial CE architecture consisted of an N-terminal fusion of an HUHe (PCV2) to nCas9 and a C-terminal fusion of a DDP to nCas9—making HUHe-nCas9-DDP. We wondered whether alternative architectures could also lead to productive click editing and whether any alternative architecture could improve efficiencies past that of our initial design. To test this, we constructed 5 additional CE architectures and compared editing efficiencies at ACTB (1 bp transversion) and DNMT1 (3 bp deletion) with a range of PBS lengths, where shorter PBS lengths may impose more stringent requirements for having more optimal CE configurations (FIG. 39A-C). While we observed productive editing with all configurations and PBS lengths across both sites, both our initial architecture (PCV2-nCas9-DDP) and other alternative architectures (e.g. PCV2-DDP-nCas9, Cas9-PCV2-DDP, etc.) led to high editing efficiencies. Notably, for a short 10 bp PBS, the PCV2-DDP-nCas9 architecture gave higher efficiencies than our initial architecture.

### Example 14: Double Click Editing and Kilobase DNA Insertion Using Click Editors and Recombinases

To test whether we could install larger segments of DNA using a “double click” approach, we pre-annealed two clkDNA templates—each containing a respective FBR (also known as a PBS) and complementary sequences encoding the desired insertion (in this instance, a BxbI attB site; sequence

(SEQ ID NO: 211))
GGCTTGTCGACGACGGCGGTCTCCGTCGTCAGGATCAT.

One of the oligos also contained an HUHe recognition sequence for template recruitment to the target site (FIG. 40A). We targeted a CE containing either fused or unfused EcKlenow or ePhi29 DDP to the ACTB locus, and observed productive click editing across each CE architecture tested at this site, with up to ~12.5% attB site installation when using a CE with an unfused EcKlenow (FIG. 40B). A tyrosine or serine recombinase can be used to integrate a donor sequence into the installed recombinase target site.

### Example 15: Click Editing Using Endogenous DNA-Dependent Polymerases

While canonical PCEs harness an exogenous natural or engineered DDP to perform the extension reaction, certain clkNA configurations may allow usage of endogenous polymerases—simplifying PCE size and/or component number. Architectures described in FIG. 10 but which omit the DDP may be used for PCEs that leverage endogenous, instead of exogenous, DDPs. To test whether endogenous DNA polymerases can be used for click editing, we targeted ACTB and DNMT1 with a PCV2-nCas9-EcKlenow, PCV2-nCas9-dKlenow, PCV2-nCas9 (no exogenous DDP fused or expressed), or nCas9 (no exogenous DDP fused or expressed), using clkDNAs with long, medium, or short PBSs (20 nt, 16 nt, and 12 nt, respectively). We observed that PCV2-nCas9 conditions could lead to comparably efficient editing as the PCV2-nCas9-EcKlenow conditions with longer PBS lengths (as well as nCas9 to a lesser extent) (FIG. 41A); however, medium and short PBS lengths led to relatively lower editing at both sites for all constructs aside from the canonical CE (PCV2-nCas9-EcKlenow). Similarly, at the VEGFA locus we observed similar editing levels with the conventional PCV2-nCas9-EcKlenow construct when compared to the PCV2-nCas9 lacking a fused exogenous DDP (or to a slightly lesser extent with nCas9) with a long 17 bp PBS (FIG. 41B). These results demonstrate that leveraging endogenous DNA-dependent polymerases for click editing is possible (via PCV2-nCas9 or nCas9) and is further enhanced with active template recruitment (e.g. via HUHes, TBPs, etc.), but this process requires a long PBS to enable editing levels comparable to our PCE1 editor which uses exogenous DNA-dependent polymerases.

Despite the success of CEs which utilize endogenous polymerases, there may be certain limitations with this approach. First, due to the general requirement for longer PBSs on the clkDNA, the use of endogenous polymerases may lead to interactions between the spacer of the gRNA and the PBS of the clkDNA (so-called gRNA-clkDNA sequestration) and/or potentially gRNA cleavage from RNAseH1 due to the RNA:DNA duplex generated between the gRNA spacer and the PBS of the clkDNA, which limits maximal editing efficiencies. To obviate sgRNA-PBS sequestration in canonical CEs (e.g. HUH-nCas9-DDP) or CEs that use endogenous polymerases (e.g. HUH-nCas9), precomplexed CE ribonucleoproteins (RNPs) can be used. Second, the differing native expression levels of a variety of endogenous DNA polymerases, each with their own processivity and fidelity, in diverse cell types may differentially affect click editing efficiency and fidelity. Third, the absolute efficiency ceiling and compatibility with designer chemical modifications on the clkDNA may be lower compared to CE constructs that utilize engineered exogenous DNA-dependent polymerases with desirable properties (due to incompatibility of endogenous human DDPs with certain chemical modifications).

### Example 16: Template Jumping Polymerase Click Editing Methodology

In addition to canonical and dual flap (double click) polymerase click editing methodologies, “template jumping” polymerase click editing can be performed to install or replace DNA segments (FIGS. 42A-C), similar to as previously described (Zheng & Liu, Nat Commun. 2023 Jun. 8; 14(1):3369). In a click editing template jumping approach, a clkNA is designed that contains a first PBS (PBS1), an edit of interest to be inserted, and the reverse complement of a second PBS (rcPBS2) (FIG. 42A). Two guide RNAs are used to induce nicking at two separate sites. During a click editing template jumping reaction, the PBS1 sequence anneals to the genomic flap released by the DNA binding domain (e.g. nCas9) at the first nicked site and an endogenous or exogenous DDP carries out clkDNA-templated DNA polymerization to synthesize the strand complementary to the clkDNA, which is extended from the genomic 3′ flap (FIG. 42B). The synthesized strand (i.e., the extended genomic flap) contains the desired edited sequence as well as PBS2. Next, once the clkDNA dissociates from the synthesized strand, the PBS2 sequence from the synthesizes strand can then hybridize to the genomic flap released by the DNA binding domain at the second nick site, and an endogenous or exogenous DDP carries out second-strand synthesis. The sequence between the two nicks is replaced by the newly synthesized sequence encoded originally on the clkDNA, following DNA repair. Notably, an alternative version where PBS1 binds the genomic flap at the second nick site instead of the first nick site is also possible (FIG. 42C)

To test this approach using exogenous or endogenous polymerases, we attempted to replace a 90 bp segment of DNA at the AAVS1 locus with a 40 bp LoxP sequence using nCas9 only or a CE containing PCV2-nCas9-EcKlenow or PCV2-nCas9-ePhi29. The clkDNA contained a PCV2 recognition site, a 16 bp PBS1, the reverse complement of a 40 bp LoxP sequence, and a 17 bp rcPBS2. In all conditions we observed productive CE-mediated installation of the 40 bp LoxP sequence at the targeted site (FIG. 42D), with the ePhi29 CE reaching nearly 30% installation. Notably, a long PBS1 permitted efficient editing with the nCas9 only condition via usage of endogenous polymerases, and editing efficiencies of PCE-ePhi29 was higher than that of PCE-EcKlenow, which may be due to the superior processivity and secondary structure read through capabilities of ePhi29 over wild-type EcKlenow.

### Example 17: Opportunities for Click Editor Development

Genome writing technologies that employ DNA-dependent DNA polymerases (DDPs), as opposed to reverse transcriptases (RTs), may offer potential advantages that might result in a higher ceiling of editing efficiency and fidelity. DDPs have been shown to be capable of higher-fidelity polymerization, exhibit higher substrate processivity, and are more likely to be highly active in any cell type due to their high dNTP affinity (Berdis, Chem Rev. 2009 July; 109(7):2862-79; Ponnienselvan et al., bioRxiv [Preprint]. 2023 Oct. 29:2023.10.21.563443, Johansson and Dixon, Cold Spring Harb Perspect Biol. 2013 Jun. 1; 5(6):a012799, Levesque et al, bioRxiv [Preprint]. 2023 doi.org/10.1101/2023.10.22.563434). Our results demonstrate that first generation click editors that utilize a wild-type DDP have similar or greater efficiency when compared to first generation prime editors (PE1) that utilize a wild-type RT (Anzalone et al., Nature. 2019 December; 576(7785):149-157), whereas subsequent prime editors (PE2) that utilize engineered RT domains exhibited superior efficiencies. Additional factors that may have impacted our comparison of CEs and PEs include:
1. Cell type used for comparisons. We utilized HEK 293T cells for our experiments, which divide rapidly and therefore do not have limited dNTP availability. Instead, experiments in slowly dividing or non-cycling cells may be better suited to probe the impact of DDP and RT domain affinity for dNTPs, as has been shown recently (Ponnienselvan et al., bioRxiv [Preprint]. 2023 Oct. 29:2023.10.21.563443, Levesque et al, bioRxiv [Preprint]. 2023 doi.org/10.1101/2023.10.22.563434).
2. Template recruitment. In contrast to prime editing, for which the template is recruited efficiently to the target site given physical tethering to the prime editing guide RNA (pegRNA; although decoupled pegRNAs have been shown to be effective as well (Liu et al., Nat Biotechnol. 2022 September; 40(9):1388-1393), in click editing the clkDNA DNA oligonucleotide template is recruited to the target site via HUHes (or other recruitment modules, such as telomere binding proteins or monomeric streptavidin). Improvements to template recruitment, through HUHe engineering, ortholog screening, or otherwise, should improve clkDNA template recruitment to the CE and thus click editing efficiencies. Additionally, most of our results were obtained with unmodified, or minimally modified, clkDNAs. Exploring additional modifications to the clkDNA via nucleic acid structures or chemical modifications might improve clkDNA stability, folding, and binding to the recruitment module, which together may improve click editing efficiencies.
3. DDP improvements and initiation sequence. Our experiments utilized CEs harboring wild-type DDPs. Future studies to engineer key features of DDPs in the context of CEs (such as processivity, binding affinity to the template-primer junction, etc.) should improve editing efficiencies. Moreover, DDP initiation sequence preferences17 may also differ between DDPs and RTs and could therefore impact editing efficiencies. A better understanding of these sequence biases will inform the development of optimized CEs.

TABLE 1
Plasmids used in this study

Click Editor Expression Plasmids

| plasmid | | |
|---|---|---|
| ID | plasmid description | plasmid use |
| EMK488 | pCMV-T7-PCV2-nSpCas9(H840A)- | Expresses the CE1 fusion protein used in most |
| | EcKlenow(-exo; D355A/E357A) | datasets in the manuscript |
| EMK492 | pCMV-T7-deadPCV2(Y96F)- | Control CE1 plasmid that has a catalytically |
| | nSpCas9(H840A)-EcKlenow(-exo; | inactivated PCV2-HUHe |
| | D355A/E357A) | |
| EMK500 | pCMV-T7-PCV2- | Control CE1 plasmid that has a catalytically |
| | deadSpCas9(D10A/H840A)-EcKlenow(- | inactivated Cas9 |
| | exo; D355A/E357A) | |
| JO520 | pCMV-T7-PCV2-XTEN24-nCas9(H840A)- | Control CE1 plasmid that has a catalytically |
| | SGGSx2-sv40NLS-SGGSx2- | inactivated Ecklenow |
| | dKlenow(D355A, D357A, D705A, D882A)- | |
| | SV40NLS | |
| JO595 | pCMV-T7-PCV2-nSpCas9(H840A)- | CE1 plasmid expressing Taq Stoffel |
| | TaqStoffel | polymerase |
| JO608 | pCMV-T7-DCV-XTEN24-nCas9(H840A)- | CE1 plasmid expressing DCV as an HUHe |
| | SGGSx2-SV40NLS-SGGSx2-Klenow(E. coli)- | |
| | SV40NLS | |
| JO661 | pCMV-T7-MSMV-XTEN24-nCas9(H840A)- | CE1 plasmid expressing MSMV as an HUHe |
| | SGGSx2-SV40NLS-SGGSx2-Klenow(E. coli)- | |
| | SV40NLS | |
| JO604 | pCMV-T7-Tral-XTEN24-nCas9(H840A)- | CE1 plasmid expressing Tral as an HUHe |
| | SGGSx2-SV40NLS-SGGSx2-Klenow(E. coli)- | |
| | SV40NLS | |
| JO606 | pCMV-T7-RepBm-XTEN24-nCas9(H840A)- | CE1 plasmid expressing RepBm as an HUHe |
| | SGGSx2-SV40NLS-SGGSx2-Klenow(E. coli)- | |
| | SV40NLS | |
| JO610 | pCMV-T7-FBYNV-XTEN24-nCas9(H840A)- | CE1 plasmid expressing FBYNV as an HUHe |
| | SGGSx2-SV40NLS-SGGSx2-Klenow(E. coli)- | |
| | SV40NLS | |
| JO659 | pCMV-T7-TGMV-XTEN24-nCas9(H840A)- | CE1 plasmid expressing TGMV as an HUHe |
| | SGGSx2-SV40NLS-SGGSx2-Klenow(E. coli)- | |
| | SV40NLS | |
| JO1271 | pCMV-T7-BDFV-XTEN24-nCas9(H840A)- | CE1 plasmid expressing BDFV as an HUHe |
| | SGGSx2-SV40NLS-SGGSx2-Klenow(E. coli)- | |
| | SV40NLS | |
| JO1262 | pCMV-T7-CoCV-XTEN24-nCas9(H840A)- | CE1 plasmid expressing CoCV as an HUHe |
| | SGGSx2-SV40NLS-SGGSx2-Klenow(E. coli)- | |
| | SV40NLS | |
| JO1263 | pCMV-T7-FiCV-XTEN24-nCas9(H840A)- | CE1 plasmid expressing FiCV as an HUHe |
| | SGGSx2-SV40NLS-SGGSx2-Klenow(E. coli)- | |
| | SV40NLS | |
| JO1279 | pCMV-T7-GuCV-XTEN24-nCas9(H840A)- | CE1 plasmid expressing GuCV as an HUHe |
| | SGGSx2-SV40NLS-SGGSx2-Klenow(E. coli)- | |
| | SV40NLS | |
| JO1315 | pCMV-T7-PCV1-XTEN24-nCas9(H840A)- | CE1 plasmid expressing PCV1 as an HUHe |
| | SGGSx2-SV40NLS-SGGSx2-Klenow(E. coli)- | |
| | SV40NLS | |
| JO1281 | pCMV-T7-NG12-XTEN24-nCas9(H840A)- | CE1 plasmid expressing NG12 as an HUHe |
| | SGGSx2-SV40NLS-SGGSx2-Klenow(E. coli)- | |
| | SV40NLS | |
| JO1283 | pCMV-T7-NG14-XTEN24-nCas9(H840A)- | CE1 plasmid expressing NG14 as an HUHe |
| | SGGSx2-SV40NLS-SGGSx2-Klenow(E. coli)- | |
| | SV40NLS | |
| JO1266 | pCMV-T7-PK5006-XTEN24-nCas9(H840A)- | CE1 plasmid expressing PK5006 as an HUHe |
| | SGGSx2-SV40NLS-SGGSx2-Klenow(E. coli)- | |
| | SV40NLS | |
| JO1287 | pCMV-T7-PK5034-XTEN24-nCas9(H840A)- | CE1 plasmid expressing PK5034 as an HUHe |
| | SGGSx2-SV40NLS-SGGSx2-Klenow(E. coli)- | |
| | SV40NLS | |
| JO1290 | pCMV-T7-PK5222-XTEN24-nCas9(H840A)- | CE1 plasmid expressing PK5222 as an HUHe |
| | SGGSx2-SV40NLS-SGGSx2-Klenow(E. coli)- | |
| | SV40NLS | |
| JO1324 | pCMV-T7-PK5510-XTEN24-nCas9(H840A)- | CE1 plasmid expressing PK5510 as an HUHe |
| | SGGSx2-SV40NLS-SGGSx2-Klenow(E. coli)- | |
| | SV40NLS | |
| JO1298 | pCMV-T7-T25-XTEN24-nCas9(H840A)- | CE1 plasmid expressing T25 as an HUHe |
| | SGGSx2-SV40NLS-SGGSx2-Klenow(E. coli)- | |
| | SV40NLS | |
| JO1273 | pCMV-T7-Chimp11-XTEN24- | CE1 plasmid expressing Chimp11 as an HUHe |
| | nCas9(H840A)-SGGSx2-SV40NLS-SGGSx2- | |
| | Klenow(E. coli)-SV40NLS | |
| JO1269 | pCMV-T7-StCV-XTEN24-nCas9(H840A)- | CE1 plasmid expressing StCV as an HUHe |
| | SGGSx2-SV40NLS-SGGSx2-Klenow(E. coli)- | |
| | SV40NLS | |
| JO1261 | pCMV-T7-CaCV-XTEN24-nCas9(H840A)- | CE1 plasmid expressing CaCV as an HUHe |
| | SGGSx2-SV40NLS-SGGSx2-Klenow(E. coli)- | |
| | SV40NLS | |
| JO1275 | pCMV-T7-Chimp17-XTEN24- | CE1 plasmid expressing Chimp17 as an HUHe |
| | nCas9(H840A)-SGGSx2-SV40NLS-SGGSx2- | |
| | Klenow(E. coli)-SV40NLS | |
| JO1277 | pCMV-T7-GoCV-XTEN24-nCas9(H840A)- | CE1 plasmid expressing GoCV as an HUHe |
| | SGGSx2-SV40NLS-SGGSx2-Klenow(E. coli)- | |
| | SV40NLS | |
| JO1270 | pCMV-T7-SwCV-XTEN24-nCas9(H840A)- | CE1 plasmid expressing SwCV as an HUHe |
| | SGGSx2-SV40NLS-SGGSx2-Klenow(E. coli)- | |
| | SV40NLS | |
| JO1264 | pCMV-T7-NG13-XTEN24-nCas9(H840A)- | CE1 plasmid expressing NG13 as an HUHe |
| | SGGSx2-SV40NLS-SGGSx2-Klenow(E. coli)- | |
| | SV40NLS | |
| JO1268 | pCMV-T7-RaCV-XTEN24-nCas9(H840A)- | CE1 plasmid expressing RaCV as an HUHe |
| | SGGSx2-SV40NLS-SGGSx2-Klenow(E. coli)- | |
| | SV40NLS | |
| JO1293 | pCMV-T7-Cdc13-XTEN24-nCas9(H840A)- | CE1 plasmid expressing Cdc13 as a TBP for |
| | SGGSx2-SV40NLS-SGGSx2-Klenow(E. coli)- | clkDNA recruitment |
| | SV40NLS | |
| JO1329 | pCMV-T7-Cdc13(Y556A)-XTEN24- | CE1 plasmid expressing Cdc13(Y556A) as a TBP |
| | nCas9(H840A)-SGGSx2-SV40NLS-SGGSx2- | for clkDNA recruitment |
| | Klenow(E. coli)-SV40NLS | |
| JO574 | pCMV-T7-PCV2-XTEN24-nCas9(H840A)- | CE1 plasmid expressing Polymerase Beta |
| | SGGSx2-SV40NLS-SGGSx2-PolBeta- | |
| | SV40NLS | |
| JO582 | pCMV-T7-PCV2-XTEN24-nCas9(H840A)- | CE1 plasmid expressing Phi29 (D169A) DNA |
| | SGGSx2-SV40NLS-SGGSx2-Phi29(D169A)- | polymerase |
| | SV40NLS | |
| JO584 | pCMV-T7-PCV2-XTEN24-nCas9(H840A)- | CE1 plasmid expressing Sequenase DNA |
| | SGGSx2-SV40NLS-SGGSx2-Sequenase(T7)- | polymerase |
| | SV40NLS | |
| JO638 | pCMV-T7-PCV2-XTEN24-nCas9(H840A)- | CE1 plasmid expressing T4 DNA polymerase |
| | SGGSx2-SV40NLS-SGGSx2- | |
| | T4pol(D189A, E191A, L412M)-SV40NLS | |
| LM2938 | pCMV-T7-PCV2-nCas9(H840A)-Phi29 | CE1 plasmid encoding Phi29 DNA polymerase |
| LM2990 | pCMV-T7-PCV2-nCas9(H840A)- | CE1 plasmid encoding ePhi29 DNA polymerase |
| | ePhi29(M8R, V51A, M97T, G197D, E221K, | |
| | Q497P, K512E, F526L) | |
| LM2993 | pCMV-T7-PCV2-nCas9(H840A)- | CE1 plasmid encoding ePhi29 (D169A) DNA |
| | ePhi29(D169A, M8R, V51A, M97T, G197D, | polymerase |
| | E221K, Q497P, K512E, F526L) | |
| LM2963 | pCMV-T7-PCV2-nCas9(H840A)-B103 | CE1 plasmid encoding B103 DNA polymerase |
| LM2702 | pCMV-T7-PCV2-nCas9(H840A)-BPNLS | PCV2-nCas9 plasmid used for unfused |
| | | architecture |
| LM2705 | pCMV-T7-PCV2-nCas9(H840A)-BPNLS- | PCV2-nCas9-CC (N6) plasmid used for |
| | CC(N6) | recruited architecture |
| LM2692 | pCMV-T7-EcKlenow-BPNLS | Plamid expressing Ecklenow for unfused |
| | | architecture |
| LM2759 | pCMV-T7-Phi29-BPNLS | Plamid expressing Phi29 for unfused |
| | | architecture |
| LM2708 | pCMV-T7-Phi29(D169A)-BPNLS | Plamid expressing Phi29(D169A) for unfused |
| | | architecture |
| LM2995 | pCMV-T7-ePhi29-BPNLS (M8R, V51A, | Plamid expressing ePhi29 for unfused |
| | M97T, G197D, E221K, Q497P, K512E, | architecture |
| | F526L) | |
| LM2802 | pCMV-T7-ePhi29-BPNLS (D169A, M8R, | Plamid expressing ePhi29(D169A) for unfused |
| | V51A, M97T, G197D, E221K, Q497P, | architecture |
| | K512E, F526L) | |
| LM2907 | pCMV-T7-B103-BPNLS | Plamid expressing B103 for unfused |
| | | architecture |
| LM2696 | pCMV-T7-Ecklenow-BPNLS-CC(N5) | Plamid expressing Ecklenow-CC(N5) for |
| | | recruited architecture |
| LM2761 | pCMV-T7-Phi29-BPNLS-CC(N5) | Plamid expressing Phi29-CC(N5) for recruited |
| | | architecture |
| LM2726 | pCMV-T7-Phi29(D169A)-BPNLS-CC | Plamid expressing Phi29(D169A)-CC(N5) for |
| | | recruited architecture |
| LM2982 | pCMV-T7-ePhi29-BPNLS-CC(N5) (M8R, | Plamid expressing ePhi29-CC(N5) for recruited |
| | V51A, M97T, G197D, E221K, Q497P, | architecture |
| | K512E, F5) | |
| LM2719 | pCMV-T7-ePhi29-BPNLS-CC(N5) (D169A, | Plamid expressing ePhi29(D169A)-CC(N5) for |
| | V51A, M97T, G197D, E221K, Q497P, | recruited architecture |
| | K512E, F5) | |
| LM2946 | pCMV-T7-B103-BPNLS-CC(N5) | Plamid expressing B103-CC(N5) for recruited |
| | | architecture |
| DRR921 | LTR-pEF1a-PCV2-nCas9(H840A)- | Lentiviral plasmid of CE1 fusion used to |
| | Ecklenow(-exo; D355A/E357A)-P2A-PuroR | transduced fibroblasts |
| DRR1012 | pCMV-T7-PCV2-nCas9(H840A)-M- | CE1 plasmid encoding wild-type M-MLV |
| | MLV(PE1) | reverse transcriptase (found in PE1) |
| JO586 | pCMV-T7-PCV2-nCas9(H840A)-M- | CE1 plasmid encoding an engineered |
| | MLV(PE2) | pentamutant M-MMLV revese transcriptase |
| | | (found in PE2) |
| MLE194 | pCMV-T7-PCV2-nSaCas9(N580A)- | CE1 plasmid encoding SaCas9 for DNA |
| | EcKlenow(-exo; D355A/E357A) | targeting |
| JO1391 | pCMV-T7-mSA-nSpCas9(H840A)- | CE1 plasmid encoding mSA for recruitment of |
| | EcKlenow(-exo; D355A/E357A) | the clkDNA |
| JO1398 | pCMV-T7-mSA-nCas9(H840A)- | CE1 plasmid encoding mSA for recruitment of |
| | ePhi29(D169A, M8R, V51A, M97T, G197D, | the clkDNA and ePhi29(D169A) DNA |
| | E221K, Q497P, K512E, F526L) | polymerase |
| JO1421 | pCMV-T7-PCV2-EcKlenow(-exo; | PCV2-EcKlenow plasmid for unfused |
| | D355A/E357A) | architecture |
| JO1420 | pCMV-T7-PCV2-ePhi29(D169A, M8R, | PCV2-ePhi29(D169A) plasmid for unfused |
| | V51A, M97T, G197D, E221K, Q497P, | architecture |
| | K512E, F526L) | |
| JO1401 | pCMV-T7-mSA-EcKlenow(-exo; | mSA-EcKlenow plasmid for unfused |
| | D355A/E357A) | architecture |
| JO1411 | pCMV-T7-mSA-ePhi29(D169A, M8R, | mSA-ePhi29(D169A) plasmid for unfused |
| | V51A, M97T, G197D, E221K, Q497P, | architecture |
| | K512E, F526L) | |
| CJT472 | T7-5′UTR-PCV2-nCas9(H840A)- | Vector for producing CE1 mRNA (PCV2-nCas9- |
| | EcKlenow(exo-)-3′UTR-polyA | Ecklenow) |
| JO1369 | pCMV-T7-MCP(N55K)-nCas9-EcKlenow | CE plasmid encoding MCP(N55K) for |
| | | recruitment of the clkDNA |
| JO1379 | pCMV-T7-MCP(N55K)-nCas9- | CE plasmid encoding MCP(N55K) for |
| | ePhi29(D169A) | recruitment of the clkDNA and ePhi29(D169A) |
| | | DNA polymerase |
| JO1357 | pCMV-T7-MCP(N55K)-EcKlenow | MCP(N55K)-EcKlenow plasmid for fused |
| | | archtecture |
| JO1365 | pCMV-T7-MCP(N55K)-ePhi28(D169A) | MCP(N55K)-ePhi29 for unfused architecture |
| JO1412 | pCMV-T7-MCP-nCas9-EcKlenow | CE plasmid encoding MCP for recruitment of |
| | | the clkDNA |
| JO1415 | pCMV-T7-MCP-nCas9-ePhi29(D169A) | CE plasmid encoding MCP for recruitment of |
| | | the clkDNA and ePhi29(D169A) DNA |
| | | polymerase |
| DRR966 | pCMV-T7-nCas9-Ecklenow-PCV2 | C-P-H architecture plasmid |
| DRR1054 | pCMV-T7-nCas9-PCV2-Ecklenow | C-H-P architecture plasmid |
| DRR1056 | pCMV-T7-PCV2-Ecklenow-nCas9 | H-P-C architecture plasmid |
| DRR1018 | pCMV-T7-EcKlenow-PCV2-nCas9 | P-H-C architecture plasmid |
| DRR887 | pCMV-T7-Ecklenow-nCas9-PCV2 | P-C-H architecture plasmid |
| CJT476 | T7-5′UTR-NLS-Bxbl-3′UTR-polyA | Vector for producing Bxbl mRNA |

| | | | |
|---|---|---|---|
| plasmid | Addgene | | |
| ID | ID | plasmid description | plasmid use |

Nuclease and Nickase Expression Plasmids

| | | | |
|---|---|---|---|
| HES140 | 208976 | pCMV-T7- | SpCas9 nickase (HNH inactive), nicks the non-target |
| | | (FZ)SpCas9(H840A)- | strand |
| | | BPNLS(SV40)-3xFLAG- | |
| | | P2A-EGFP | |
| RTW3027 | 139987 | pCMV-T7-(FZ)SpCas9- | SpCas9 nuclease, induces DNA double-strand breaks |
| | | BPNLS(SV40)-3xFLAG- | and indels at guide specified target site |
| | | P2A-EGFP | |

Prime Editor Expression Plasmids

| | | | |
|---|---|---|---|
| pCMV-PE1 | 132774 | pCMV-T7-NLS- | Prime editing in mammalian cells. This plasmid is used by |
| (MNW94) | | nSpCas9(H840A)- | PE1 |
| | | M-MLV-RT-NLS | |
| pCMV-PE2 | 132775 | pCMV-T7-NLS- | Prime editing in mammalian cells. This plasmid is used by |
| (MNW95) | | nSpCas9(H840A)- | PE2, PE3, and PE3b |
| | | M-MLV- | |
| | | RT(D200N, T306K, | |
| | | W313F, T330P, | |
| | | L603W)-NLS | |

TABLE 2
gRNAs used in this study
Primary click editing gRNAs used in this study

| Plasmid | target | | SEQ ID | PAM |
|---|---|---|---|---|
| ID | site name | spacer sequence | NO(s): | (NGGN) |
| CJT296 | DNMT1 | GATTCCTGGTGCCAGAAACA | 212 | GGGG |
| LM2363 | RNF2 | GTCATCTTAGTCATTACCTG | 213 | AGGT |
| CJT335 | ACTB | GCTATTCTCGCAGCTCACCA | 214 | TGGA |
| LM2367 | TGFBI | CAGGCCTCAGCTTCTCCGTG | 215 | CGGT |
| JO617 | VEGFA | GATGTCTGCAGGCCAGATGA | 216 | GGGC |
| JO620 | IL2RB | CCAGGTGTCTTTCAAAGTAG | 217 | TGGG |
| JO1209 | PRNP | GCAGTGGTGGGGGGCCTTGG | 218 | CGGC |
| LM2372 | GJB2 | ACGCTGCAGACGATCCTGGG | 219 | GGG |
| J0601 | HEK3 site | GGGCCCAGACTGAGCACGTGA | 220 | TGGC |
| EMK426 | EMX1 | GCAACCACAAACCCACGAGGG | 221 | CAGAGT |

Nicking gRNAs used in this study

| | | position | | | |
|---|---|---|---|---|---|
| | | compared | | | |
| | target | to | | | |
| Plasmid | site | primary | PAM | | SEQ ID |
| ID | name | guide | (NGGN) | spacer sequence | NO(S): |
| CJT297 | DNMT1 | plus 49 | AGGA | CCCTTCAGCTAAAATAAAGG | 222 |
| J0598 | DNMT1 | plus 1 | TGG | GTGCCCTCCCGTCACCGTTTC | 223 |
| JO550 | RNF2 | plus 5 | AGGT | TGAGTTACAACGAACACCGC | 224 |
| LM2375 | RNF2 | plus 41 | GGG | GTCAACCATTAAGCAAAACAT | 225 |
| CJT334 | ACTB | plus 48 | CGGA | GAAGCCGGCCTTGCACATGC | 226 |
| JO1175 | ACTB | plus 5 | TGGT | CGCGGCGATATCATCATCGA | 227 |
| JO1177 | ACTB | plus 63 | CGGC | GGCATCGTCGCCCGCGAAGC | 228 |
| JO1179 | ACTB | plus 80 | GGGC | GGAGGGGAAGACGGCCCGGG | 229 |
| JO1181 | ACTB | plus 81 | GGGG | TGGAGGGGAAGACGGCCCGG | 230 |
| JO1233 | TGFBI | plus 4 | CGGA | AGCTGTACACGGACCATGCA | 231 |
| JO1211 | PRNP | plus 72 | CGGT | GCATGTTTTCACGATAGTAA | 232 |
| JO622 | IL2RB | plus 55 | TGGA | CTCCCTCCAAGTTGTCCACG | 233 |
| LM2381 | GJB2 | plus 29 | TGG | ACGCTGCAGACGATCCTGGG | 219 |
| MLE84 | GJB2 | minus 74 | AGG | AAGACGAATGCACACAACAC | 234 |
| J0561 | HEK3 | plus 90 | AGG | GGTCAACCAGTATCCCGGTGC | 235 |
| | site | | | | |
| MLE261 | EMX1 | plus 60 | CAGAGT | GAAGCCTGGCCAGGGAGTGGC | 236 |
| MLE265 | EMX1 | plus 69 | GGGAGT | GGCCTCCCCAAAGCCTGGCCA | 237 |

Prime editing guide RNAs (pegRNAs)

| site | | SEQ ID | PAM | | SEQ ID |
|---|---|---|---|---|---|
| name | spacer sequence | NO(s): | (NGGN) | target molecule | NO(s): |
| LM3000 | GCTATTCTCGCAGCTCAC | 214 | TGGA | ATATCATCATCGATGGTGAGC | 238 |
| | CA | | | TGCGAGAA | |
| LM3001 | GCTATTCTCGCAGCTCAC | 214 | TGGA | CCATCGATGATGATGAGCTGC | 239 |
| | CA | | | GAGAA | |
| LM3002 | GCTATTCTCGCAGCTCAC | 214 | TGGA | CCATCGATGATGAGCTGCGAG | 240 |
| | CA | | | AA | |
| LM3004 | GATTCCTGGTGCCAGAAA | 212 | GGGG | TCCCGTCACCGTTTCTGGCAC | 241 |
| | CA | | | CAGG | |
| LM3008 | GATGTCTGCAGGCCAGAT | 216 | GGGC | AATGTGCCATCTGGCGTACGC | 242 |
| | GA | | | ATCTGGCCTGCAGA | |
| LM3012 | GTCATCTTAGTCATTACC | 213 | AGGT | AACGAACACCGCAGGTAATGA | 243 |
| | TG | | | CTAAGATG | |

PCV2 pseudosite targeting guide RNAs

| site | | PAM | | SEQ ID |
|---|---|---|---|---|
| name | Target | (NGGN) | spacer sequnce | NO(s): |
| EMK936 | PCV2 pseudosite 1 | NGG | AATGTAAGTATTACCAGCAA | 244 |
| EMK938 | PCV2 pseudosite 2 | NGG | TTGGGAAGTATTACCAGCAT | 245 |
| EMK939 | PCV2 pseudosite 3 | NGG | TITCTAAGTATTACCAGCCC | 246 |
| EMK940 | PCV2 pseudosite 4 | NGG | CACACAAGTATTACCAGCTT | 247 |
| EMK941 | PCV2 pseudosite 5 | NGG | CTAACCAAGTATTACCAGCA | 248 |
| EMK942 | PCV2 pseudosite 6 | NGG | AAGGCTAAGTATTACCAGCC | 249 |
| EMK944 | PCV2 pseudosite 7 | NGG | CCTTACAAGTATTACCAGCC | 250 |
| EMK945 | PCV2 pseudosite 8 | NGG | GGCAAGTATTACCAGCCCTA | 251 |
| EMK946 | PCV2 pseudosite 9 | NGG | AAAAAGTATTACCAGCAGAA | 252 |
| EMK948 | PCV2 pseudosite 10 | NGG | ATGAAGTATTACCAGCATCC | 253 |
| EMK951 | PCV2 pseudosite 11 | NGG | GGGCAAGTATTACCAGCCCT | 254 |
| EMK952 | PCV2 pseudosite 12 | NGG | CGTAAAGTATTACCAGCCGT | 255 |
| EMK953 | PCV2 pseudosite 13 | NGG | TGGGAAGTATTACCAGCATA | 256 |
| EMK957 | PCV2 pseudosite 14 | NGG | AAGTATTACCAGCAGAATGG | 257 |
| EMK958 | PCV2 pseudosite 15 | NGG | AAGTATTACCAGCCCTTCAT | 258 |
| EMK959 | PCV2 pseudosite 16 | NGG | AAGTATTACCAGCAATTATT | 259 |

TABLE 3
Oligonucleotides used for NGS library preparation in this study

| primer | primer | | SEQ ID |
|---|---|---|---|
| ID | description | full primer sequence | NO(s): |
| oCT1192 | p5 flap primer | ACACTCTTTCCCTACACGACGCTCTTCCGATCTCACAACAG | 260 |
| | DNMT1 (+3-5 | CTTCATGTCAGCC | |
| | AGGdel) | | |
| oCT1193 | p7 flap primer | GACTGGAGTTCAGACGTGTGCTCTTCCGATCTCGTTAATGT | 261 |
| | DNMT1 (+3-5 | TTCCTGATGGTCC | |
| | AGGdel) | | |
| oJO336 | p5 flap primer | ACACTCTTTCCCTACACGACGCTCTTCCGATCTGACCATAG | 262 |
| | RNF2 (+4 A>C) | CACTTCCCTTCC | |
| oJO337 | p7 flap primer | GACTGGAGTTCAGACGTGTGCTCTTCCGATCTTTAGCCAAC | 263 |
| | RNF2 (+4 A>C) | ATACAGAAGTCAGG | |
| oCT1321 | p5 flap primer | ACACTCTTTCCCTACACGACGCTCTTCCGATCTGGCGACTT | 264 |
| | ACTB (+5 G>C) | CGGCTCACAGC | |
| oCT1322 | p7 flap primer | GACTGGAGTTCAGACGTGTGCTCTTCCGATCTACCCAGCCA | 265 |
| | ACTB (+5 G>C) | GCTCCCCTAC | |
| oKAC1593 | p5 flap primer | ACACTCTTTCCCTACACGACGCTCTTCCGATCTCGAAGATG | 266 |
| | TGFB (+4 AT | GTGAAGCTGCCG | |
| | ins | | |
| oKAC1594 | p7 flap primer | GACTGGAGTTCAGACGTGTGCTCTTCCGATCTCATCCCTCC | 267 |
| | TGFB (+4 AT | TTCTGTCTTCTG | |
| | ins | | |
| oJO391 | p5 flap primer | ACACTCTTTCCCTACACGACGCTCTTCCGATCTACTTGGTG | 268 |
| | VEGFA (+5 | CCAAATTCTTCTCC | |
| | G>T) | | |
| oJO392 | p7 flap primer | GACTGGAGTTCAGACGTGTGCTCTTCCGATCTAGAGGGAAT | 269 |
| | VEGFA (+5 | GGGCTTTGGA | |
| | G>T) | | |
| oJO1402 | p5 flap primer | ACACTCTTTCCCTACACGACGCTCTTCCGATCTGTCAGTGG | 270 |
| | PRNP (+6 G>T) | AACAAGCCGAGT | |
| oJO1403 | p7 flap primer | GACTGGAGTTCAGACGTGTGCTCTTCCGATCTGTGACCGTG | 271 |
| | PRNP (+6 G>T) | TGCTGCTTGATTG | |
| oJO1394 | p5 primer flap | ACACTCTTTCCCTACACGACGCTCTTCCGATCTGGACCAGG | 272 |
| | IL2RB (+1 T>A, | ACAGGAAGGAGGAA | |
| | +5 G>C) | | |
| oJO1395 | p7 primer flap | GACTGGAGTTCAGACGTGTGCTCTTCCGATCTGGCAGGTAC | 273 |
| | IL2RB (+1 T>A, | AAAGTGGGAGG | |
| | +5 G>C) | | |
| oJO338 | p5 primer | ACACTCTTTCCCTACACGACGCTCTTCCGATCTCTGCTGCA | 274 |
| | flap HEK3 | AGTAAGCATGC | |
| | site | | |
| oJO339 | p7 primer | GACTGGAGTTCAGACGTGTGCTCTTCCGATCTCCTCCCTAG | 275 |
| | flap HEK3 | GTGCTGGCTTC | |
| | site | | |
| oRW1323 | p5 flap | ACACTCTTTCCCTACACGACGCTCTTCCGATCTGAAGCAGG | 276 |
| | primer for | CCAATGGGGAGG | |
| | EMX1 (+6 | | |
| | G>C) | | |
| oRW1385 | p7 flap | GACTGGAGTTCAGACGTGTGCTCTTCCGATCTCTTGTCCCT | 277 |
| | primer for | CTGTCAATGGCGG | |
| | EMX1 (+6 | | |
| | G>C) | | |
| oEMK1143 | p5 flap primer | ACACTCTTTCCCTACACGACGCTCTTCCGATCTTCTGTGAG | 278 |
| | ACTB off | AGGGGTGGAGCC | |
| | target 9 | | |
| oEMK1144 | p7 flap primer | GACTGGAGTTCAGACGTGTGCTCTTCCGATCTCCAGGCTAC | 279 |
| | ACTB off | TGCAGTAGGGGA | |
| | target 9 | | |
| oEMK1145 | p5 flap primer | ACACTCTTTCCCTACACGACGCTCTTCCGATCTGTGTCTCT | 280 |
| | ACTB off | GGCCTGTCCTGC | |
| | target 8 | | |
| oEMK1146 | p7 flap primer | GACTGGAGTTCAGACGTGTGCTCTTCCGATCTCCCATGAGG | 281 |
| | ACTB off | TGTGGCAAGGA | |
| | target 8 | | |
| oEMK1147 | p5 flap primer | ACACTCTTTCCCTACACGACGCTCTTCCGATCTTGCCAGTC | 282 |
| | ACTB off | ACCCAAGTCTGG | |
| | target 7 | | |
| oEMK1148 | p7 flap primer | GACTGGAGTTCAGACGTGTGCTCTTCCGATCTTCTTGACTG | 283 |
| | ACTB off | TGGCCTCCAGA | |
| | target 7 | | |
| oEMK1149 | p5 flap primer | ACACTCTTTCCCTACACGACGCTCTTCCGATCTGCTGCTCA | 284 |
| | ACTB off | GGCCTTTGACCA | |
| | target 6 | | |
| oEMK1150 | p7 flap primer | GACTGGAGTTCAGACGTGTGCTCTTCCGATCTCCAACCTGC | 285 |
| | ACTB off | GGCCTCACAAT | |
| | target 6 | | |
| oEMK1151 | p5 flap primer | ACACTCTTTCCCTACACGACGCTCTTCCGATCTATTGCAAC | 286 |
| | ACTB off | AGGGGCAGGTCC | |
| | target 5 | | |
| oEMK1152 | p7 flap primer | GACTGGAGTTCAGACGTGTGCTCTTCCGATCTTGCAGAACA | 287 |
| | ACTB off | GGCTTCCCACG | |
| | target 5 | | |
| oEMK1155 | p5 flap primer | ACACTCTTTCCCTACACGACGCTCTTCCGATCTGGGTCTCT | 288 |
| | ACTB off | TCTCGCTTGGGC | |
| | target 4 | | |
| oEMK1156 | p7 flap primer | GACTGGAGTTCAGACGTGTGCTCTTCCGATCTGCGGGCAGA | 289 |
| | ACTB off | TCACTTGAGCC | |
| | target 4 | | |
| oEMK1157 | p5 flap primer | ACACTCTTTCCCTACACGACGCTCTTCCGATCTAGCTGCTC | 290 |
| | ACTB off | TTTGCCAACCCT | |
| | target 3 | | |
| oEMK1158 | p7 flap primer | GACTGGAGTTCAGACGTGTGCTCTTCCGATCTCGGAGTCAG | 291 |
| | ACTB off | GCCTAACGCTG | |
| | target 3 | | |
| oEMK1163 | p5 flap primer | ACACTCTTTCCCTACACGACGCTCTTCCGATCTAGGAATGT | 292 |
| | ACTB off | GCCCTCGGTTGC | |
| | target 2 | | |
| oEMK1164 | p7 flap primer | GACTGGAGTTCAGACGTGTGCTCTTCCGATCTAGTTGGGTG | 293 |
| | ACTB off | CTTCTCCAGGC | |
| | target 2 | | |
| oEMK1165 | p5 flap primer | ACACTCTTTCCCTACACGACGCTCTTCCGATCTACTAGCCC | 294 |
| | ACTB off | CTGATGCACTGC | |
| | target 1 | | |
| oEMK1166 | p7 flap primer | GACTGGAGTTCAGACGTGTGCTCTTCCGATCTGCCCAGATT | 295 |
| | ACTB off | CAAGGTAGGGAGG | |
| | target 1 | | |
| oEMK1167 | p5 flap primer | ACACTCTTTCCCTACACGACGCTCTTCCGATCTGGTGGCAT | 296 |
| | DNMT1 off | GTGAGCTGAGGC | |
| | target 11 | | |
| oEMK1168 | p7 flap primer | GACTGGAGTTCAGACGTGTGCTCTTCCGATCTTGCCCTGCA | 297 |
| | DNMT1 off | CTACACATGTCA | |
| | target 11 | | |
| oEMK1169 | p5 flap primer | ACACTCTTTCCCTACACGACGCTCTTCCGATCTAGCTCTGC | 298 |
| | DNMT1 off | CTTCCACCCAGA | |
| | target 10 | | |
| oEMK1170 | p7 flap primer | GACTGGAGTTCAGACGTGTGCTCTTCCGATCTCTCGAAGTG | 299 |
| | DNMT1 off | CCCAGCTGGAG | |
| | target 10 | | |
| oEMK1171 | p5 flap primer | ACACTCTTTCCCTACACGACGCTCTTCCGATCTGGGTGAGT | 300 |
| | DNMT1 off | GGGGTGAGGTGA | |
| | target 9 | | |
| oEMK1172 | p7 flap primer | GACTGGAGTTCAGACGTGTGCTCTTCCGATCTTCTGCCTCC | 301 |
| | DNMT1 off | CTGAGCTTCCA | |
| | target 9 | | |
| oEMK1173 | p5 flap primer | ACACTCTTTCCCTACACGACGCTCTTCCGATCTCAGGCCCT | 302 |
| | DNMT1 off | GTCCTCCCTCAT | |
| | target 8 | | |
| oEMK1174 | p7 flap primer | GACTGGAGTTCAGACGTGTGCTCTTCCGATCTCTCACATGA | 303 |
| | DNMT1 off | GAAAGGGGCTT | |
| | target 8 | | |
| oEMK1175 | p5 flap primer | ACACTCTTTCCCTACACGACGCTCTTCCGATCTTCCCGCCC | 304 |
| | DNMT1 off | TAAGTCACCTCA | |
| | target 7 | | |
| oEMK1176 | p7 flap primer | GACTGGAGTTCAGACGTGTGCTCTTCCGATCTAGGGAAGTG | 305 |
| | DNMT1 off | GGGAACTGAACCT | |
| | target 7 | | |
| oEMK1177 | p5 flap primer | ACACTCTTTCCCTACACGACGCTCTTCCGATCTCCCTTTCC | 306 |
| | DNMT1 off | AGCCAGCCAGAC | |
| | target 6 | | |
| oEMK1178 | p7 flap primer | GACTGGAGTTCAGACGTGTGCTCTTCCGATCTACATGTGGA | 307 |
| | DNMT1 off | AATGGAGGGCC | |
| | target 6 | | |
| oEMK1179 | p5 flap primer | ACACTCTTTCCCTACACGACGCTCTTCCGATCTTCTCTGTT | 308 |
| | DNMT1 off | TGCCCTGCACCA | |
| | target 5 | | |
| oEMK1180 | p7 flap primer | GACTGGAGTTCAGACGTGTGCTCTTCCGATCTCCTACGGAA | 309 |
| | DNMT1 off | GCAACGTGGCA | |
| | target 5 | | |
| oEMK1181 | p5 flap primer | ACACTCTTTCCCTACACGACGCTCTTCCGATCTAGGTGACC | 310 |
| | DNMT1 off | AGAAAAGTCTTGT | |
| | target 4 | | |
| oEMK1182 | p7 flap primer | GACTGGAGTTCAGACGTGTGCTCTTCCGATCTTCAGTCGCA | 311 |
| | DNMT1 off | GCTCAGGTCTT | |
| | target 4 | | |
| oEMK1183 | p5 flap primer | ACACTCTTTCCCTACACGACGCTCTTCCGATCTCCTGCTCT | 312 |
| | DNMT1 off | CAGCTGCCAGTG | |
| | target 3 | | |
| oEMK1184 | p7 flap primer | GACTGGAGTTCAGACGTGTGCTCTTCCGATCTTGGAAACCA | 313 |
| | DNMT1 off | GGCACCCACAT | |
| | target 3 | | |
| oEMK1187 | p5 flap primer | ACACTCTTTCCCTACACGACGCTCTTCCGATCTCGGCACAC | 314 |
| | DNMT1 off | TATGAGGCTCCC | |
| | target 2 | | |
| oEMK1188 | p7 flap primer | GACTGGAGTTCAGACGTGTGCTCTTCCGATCTGGCCATCTT | 315 |
| | DNMT1 off | TTAGCCTGGGT | |
| | target 2 | | |
| oEMK1189 | p5 flap primer | ACACTCTTTCCCTACACGACGCTCTTCCGATCTCCAGGTCT | 316 |
| | DNMT1 off | GGGCTGGAAGAG | |
| | target 1 | | |
| oEMK1190 | p7 flap primer | GACTGGAGTTCAGACGTGTGCTCTTCCGATCTGATGTCCTC | 317 |
| | DNMT1 off | ATGGCCCCAGC | |
| | target 1 | | |
| oEMK1191 | p5 flap primer | ACACTCTTTCCCTACACGACGCTCTTCCGATCTAGAGGAGG | 318 |
| | VEGFA off | CCAAAGAGCTAGCT | |
| | target 1 | | |
| oEMK1192 | p7 flap primer | GACTGGAGTTCAGACGTGTGCTCTTCCGATCTAGTTTGGTG | 319 |
| | VEGFA off | TCTTAGTTCAGGTTGGT | |
| | target 1 | | |
| oEMK1197 | p5 flap primer | ACACTCTTTCCCTACACGACGCTCTTCCGATCTCAGAGGAT | 320 |
| | VEGFA off | GATGAAGCTGTGCT | |
| | target 2 | | |
| oEMK1198 | p7 flap primer | GACTGGAGTTCAGACGTGTGCTCTTCCGATCTGCACACACA | 321 |
| | VEGFA off | GAACAGGAGAATGA | |
| | target 2 | | |
| oEMK1199 | p5 flap primer | ACACTCTTTCCCTACACGACGCTCTTCCGATCTGTGAACCA | 322 |
| | VEGFA off | GGCAGGGATGGG | |
| | target 3 | | |
| oEMK1200 | p7 flap primer | GACTGGAGTTCAGACGTGTGCTCTTCCGATCTACAAGCCCT | 323 |
| | VEGFA off | GGAGTCCCCTT | |
| | target 3 | | |
| oEMK1201 | p5 flap primer | ACACTCTTTCCCTACACGACGCTCTTCCGATCTGGCCCAAG | 324 |
| | VEGFA off | ATGGAAGGAATGGT | |
| | target 4 | | |
| oEMK1202 | p7 flap primer | GACTGGAGTTCAGACGTGTGCTCTTCCGATCTAAGCTCAGC | 325 |
| | VEGFA off | AGGCCACCAAG | |
| | target 4 | | |
| oEMK1203 | p5 flap primer | ACACTCTTTCCCTACACGACGCTCTTCCGATCTGCCAAGGC | 326 |
| | VEGFA off | TCTGACTCGGTC | |
| | target 5 | | |
| oEMK1204 | p7 flap primer | GACTGGAGTTCAGACGTGTGCTCTTCCGATCTGCTTGGCCT | 327 |
| | VEGFA off | CCAGCTGTACC | |
| | target 5 | | |
| oEMK1205 | p5 flap primer | ACACTCTTTCCCTACACGACGCTCTTCCGATCTTCAACTCT | 328 |
| | VEGFA off | CTGGCTTTGTGACAT | |
| | target 6 | | |
| oEMK1206 | p7 flap primer | GACTGGAGTTCAGACGTGTGCTCTTCCGATCTCTCCACACC | 329 |
| | VEGFA off | ACTGGACCCCT | |
| | target 6 | | |
| oEMK1207 | p5 flap primer | ACACTCTTTCCCTACACGACGCTCTTCCGATCTTGGACCTT | 330 |
| | VEGFA off | CCCTCATGCCCA | |
| | target 7 | | |
| oEMK1208 | p7 flap primer | GACTGGAGTTCAGACGTGTGCTCTTCCGATCTCGCCTGCCA | 331 |
| | VEGFA off | TTTCCCTGTCA | |
| | target 7 | | |
| oEMK1209 | p5 flap primer | ACACTCTTTCCCTACACGACGCTCTTCCGATCTTGAGAGTG | 332 |
| | VEGFA off | GCCAGGTCTACCA | |
| | target 8 | | |
| oEMK1210 | p7 flap primer | GACTGGAGTTCAGACGTGTGCTCTTCCGATCTCCACTGGCC | 333 |
| | VEGFA off | CATGTGTCCTT | |
| | target 8 | | |
| oEMK1213 | p5 flap primer | ACACTCTTTCCCTACACGACGCTCTTCCGATCTCCCGGCAT | 334 |
| | VEGFA off | TCCTCACTTCCC | |
| | target 9 | | |
| oEMK1214 | p7 flap primer | GACTGGAGTTCAGACGTGTGCTCTTCCGATCTGTGGAGCCT | 335 |
| | VEGFA off | GCGTGTTTTGC | |
| | target 9 | | |
| oEMK717 | p5 flap primer | ACACTCTTTCCCTACACGACGCTCTTCCGATCTCATCCATA | 336 |
| | for PCV2 | GACATACTTCGACTCCC | |
| | pseudosite 01 | | |
| oEMK718 | p7 flap primer | GACTGGAGTTCAGACGTGTGCTCTTCCGATCTCCAACTTCC | 337 |
| | for PCV2 | AATGTACAGACAATCC | |
| | pseudosite 01 | | |
| oEMK721 | p5 flap primer | ACACTCTTTCCCTACACGACGCTCTTCCGATCTGACCTTGG | 338 |
| | for PCV2 | CAAGCTAACTCCTCC | |
| | pseudosite 02 | | |
| oEMK722 | p7 flap primer | GACTGGAGTTCAGACGTGTGCTCTTCCGATCTGCCTGACAA | 339 |
| | for PCV2 | GCTCTCCAGGC | |
| | pseudosite 02 | | |
| oEMK723 | p5 flap primer | ACACTCTTTCCCTACACGACGCTCTTCCGATCTGCGAGTTT | 340 |
| | for PCV2 | GCTCAACAACTCTGCC | |
| | pseudosite 03 | | |
| oEMK724 | p7 flap primer | GACTGGAGTTCAGACGTGTGCTCTTCCGATCTCACAGCTAG | 341 |
| | for PCV2 | TGAGAGGAAGCCAAG | |
| | pseudosite 03 | | |
| oEMK725 | p5 flap primer | ACACTCTTTCCCTACACGACGCTCTTCCGATCTCCATGCGG | 342 |
| | for PCV2 | AGAACTTGCACAACGT | |
| | pseudosite 04 | | |
| oEMK726 | p7 flap primer | GACTGGAGTTCAGACGTGTGCTCTTCCGATCTCACGAGAAC | 343 |
| | for PCV2 | ACCAGGTAAACAGTTC | |
| | pseudosite 04 | | |
| oEMK727 | p5 flap primer | ACACTCTTTCCCTACACGACGCTCTTCCGATCTCATCAGCC | 344 |
| | for PCV2 | CATGACCGACATGTGA | |
| | pseudosite 05 | | |
| oEMK728 | p7 flap primer | GACTGGAGTTCAGACGTGTGCTCTTCCGATCTCCTGTGGGC | 345 |
| | for PCV2 | ATTTCACTGAACATC | |
| | pseudosite 05 | | |
| oEMK729 | p5 flap primer | ACACTCTTTCCCTACACGACGCTCTTCCGATCTGAAGGTGC | 346 |
| | for PCV2 | AAGCTTCCAGGTGTC | |
| | pseudosite 06 | | |
| oEMK730 | p7 flap primer | GACTGGAGTTCAGACGTGTGCTCTTCCGATCTCCTTACAGT | 347 |
| | for PCV2 | GCGATTTAGGGCAAAC | |
| | pseudosite 06 | | |
| oEMK731 | p5 flap primer | ACACTCTTTCCCTACACGACGCTCTTCCGATCTCTATGTCT | 348 |
| | for PCV2 | CAGTGGACAAAGAGAATC | |
| | pseudosite 07 | | |
| oEMK732 | p7 flap primer | GACTGGAGTTCAGACGTGTGCTCTTCCGATCTGATTCCAAA | 349 |
| | for PCV2 | GTCAGAACATGGGAAGG | |
| | pseudosite 07 | | |
| oEMK733 | p5 flap primer | ACACTCTTTCCCTACACGACGCTCTTCCGATCTGGCTTCCT | 350 |
| | for PCV2 | TTGCAACCTCTACATG | |
| | pseudosite 08 | | |
| | and 12 | | |
| oEMK734 | p7 flap primer | GACTGGAGTTCAGACGTGTGCTCTTCCGATCTCCATAGCCA | 351 |
| | for PCV2 | TATTCCAAGTGCCCT | |
| | pseudosite 08 | | |
| | and 12 | | |
| oEMK735 | p5 flap primer | ACACTCTTTCCCTACACGACGCTCTTCCGATCTCTCATTAT | 352 |
| | for PCV2 | GGAATCATTCATGTGTTCTTATC | |
| | pseudosite 09 | | |
| | and 15 | | |
| oEMK736 | p7 flap primer | GACTGGAGTTCAGACGTGTGCTCTTCCGATCTGGAGAGGCA | 353 |
| | for PCV2 | GACAGATATGACCC | |
| | pseudosite 09 | | |
| | and 15 | | |
| oEMK739 | p5 flap primer | ACACTCTTTCCCTACACGACGCTCTTCCGATCTCATCCTTA | 354 |
| | for PCV2 | TCCCACTGCCACACAG | |
| | pseudosite 10 | | |
| oEMK740 | p7 flap primer | GACTGGAGTTCAGACGTGTGCTCTTCCGATCTGGAAATTGC | 355 |
| | for PCV2 | TGTGGTACCATGGGTA | |
| | pseudosite 10 | | |
| oEMK743 | p5 flap primer | ACACTCTTTCCCTACACGACGCTCTTCCGATCTGATTACAG | 356 |
| | for PCV2 | GCATGAGCCACCACAC | |
| | pseudosite 11 | | |
| oEMK744 | p7 flap primer | GACTGGAGTTCAGACGTGTGCTCTTCCGATCTCAGGTGATC | 357 |
| | for PCV2 | TGCCGACCTTTGC | |
| | pseudosite 11 | | |
| oEMK745 | p5 flap primer | ACACTCTTTCCCTACACGACGCTCTTCCGATCTCCAGCCTG | 358 |
| | for PCV2 | GGTGACAGAGTTAG | |
| | pseudosite 13 | | |
| oEMK746 | p7 flap primer | GACTGGAGTTCAGACGTGTGCTCTTCCGATCTCCCACCAAG | 359 |
| | for PCV2 | ATCCTGAAGCCTG | |
| | pseudosite 13 | | |
| oEMK753 | p5 flap primer | ACACTCTTTCCCTACACGACGCTCTTCCGATCTCTCACAGG | 360 |
| | for PCV2 | ACATCAGATTAAGCTGC | |
| | pseudosite 14 | | |
| oEMK754 | p7 flap primer | GACTGGAGTTCAGACGTGTGCTCTTCCGATCTCCACCACAC | 361 |
| | for PCV2 | CTGGCCTTGAAC | |
| | pseudosite 14 | | |
| oEMK755 | p5 flap primer | ACACTCTTTCCCTACACGACGCTCTTCCGATCTCTTTGATC | 362 |
| | for PCV2 | TCCCTTGGCTTGGTC | |
| | pseudosite 16 | | |
| oEMK756 | p7 flap primer | GACTGGAGTTCAGACGTGTGCTCTTCCGATCTGGTGGTCTC | 363 |
| | for PCV2 | CTCCCTTATTTGTAC | |
| | pseudosite 16 | | |

TABLE 4
standard and PS-modified clkDNA oligos used in this study

| HUH | recognition sequence |
|---|---|
| PCV2 | CTGTAAGTATTACCAGC (SEQ ID |
| | NO: 41) [INSERT PT-PBS] |
| DCV | GTTATATTATTACCGGC (SEQ ID |
| | NO: 42) [INSERT PT-PBS] |
| FBNYV | GGCTTAGTATTACCCCC (SEQ ID |
| | NO: 43) [INSERT PT-PBS] |
| RepBm | TGCTTCCGTACTACGACCCCCCA |
| | (SEQ ID NO: 44) [INSERT |
| | PT-PBS] |
| Tral | TTTGCGTGGGGTGTGGTGCT |
| | (SEQ ID NO: 45) [INSERT |
| | PT-PBS] |
| MMSV | ATAATAATATTACGCGC (SEQ ID |
| | NO: 50) [INSERT PT-PBS] |
| TGMV | CGTTTAATATTACCGGA (SEQ ID |
| | NO: 52) [INSERT PT-PBS] |
| BDFV/COCV/FiCV/GuCV/PCV1 | CTGTTAGTATTACCACG (SEQ |
| | ID NO: 364) |
| NG12/NG14/PK5006/PK5034/ | CTGTTAATACTATCACG (SEQ |
| PK5222/PK5510/T25/Chimp11 | ID NO: 365) |
| StCV, CaCV, Chimp17 | CTGTCAGTATTACCAGC (SEQ |
| | ID NO: 366) |
| GoCV, SwCV | CTGTTATTATTACCAGC (SEQ |
| | ID NO: 367) |
| NG13 | CTGTTAGTATTACCAGC (SEQ |
| | ID NO: 368) |
| RaCV | CTGTGAGTATTACCAGC (SEQ |
| | ID NO: 369) |
| Cdc13/Cdc13(Y556A) | CTGTGTGTGGGTGTGCACG |
| | (SEQ ID NO: 370) |

Minially Optimized clkDNAs with PS Linkages

| oligo | | | | SEQ ID | HUH | PT | PBS |
|---|---|---|---|---|---|---|---|
| ID | target | edit | clkDNA sequence | NO(s): | rec | length | length |
| OCT1171 | DNMT1 | 3-5 | CTGTAAGTATTACCAGCTCCCG | 371 | PCV2 | 12 | 13 |
| | | delAGG | TCACCGTTTCTGGCACCA*G*G | | | | |
| oJO332 | RNF2 | 4 A-to-C | CTGTAAGTATTACCAGCAACGA | 372 | PCV2 | 14 | 15 |
| | | | ACACCGCAGGTAATGACTAAGA | | | | |
| | | | *T*G | | | | |
| oCT1225 | DNMT1 | 3-5 | TTTGCGTGGGGTGTGGTGCTTC | 373 | Tral | 12 | 13 |
| | | delAGG | CCGTCACCGTTTCTGGCACCA* | | | | |
| | | | G*G | | | | |
| oCT1224 | DNMT1 | 3-5 | TGCTTCCGTACTACGACCCCCC | 374 | RepBm | 12 | 13 |
| | | delAGG | ATCCCGTCACCGTTTCTGGCAC | | | | |
| | | | CA*G*G | | | | |
| oCT1222 | DNMT1 | 3-5 | GTTATATTATTACCGGCTCCCG | 375 | DCV | 12 | 13 |
| | | delAGG | TCACCGTTTCTGGCACCA*G*G | | | | |
| oCT1223 | DNMT1 | 3-5 | GGCTTAGTATTACCCCCTCCCG | 376 | FBYNV | 12 | 13 |
| | | delAGG | TCACCGTTTCTGGCACCA*G*G | | | | |
| oJO435 | DNMT1 | 3-5 | CGTTTAATATTACCGGATCCCG | 377 | TGMV | 12 | 13 |
| | | delAGG | TCACCGTTTCTGGCACCA*G*G | | | | |
| oJO434 | DNMT1 | 3-5 | ATAATAATATTACGCGCTCCCG | 378 | MSMV | 12 | 13 |
| | | delAGG | TCACCGTTTCTGGCACCA*G*G | | | | |
| oJO440 | RNF2 | 4 A-to-C | TTTGCGTGGGGTGTGGTGCTAA | 379 | Tral | 14 | 15 |
| | | | CGAACACCGCAGGTAATGACTA | | | | |
| | | | AGA*T*G | | | | |
| oJO439 | RNF2 | 4 A-to-C | TGCTTCCGTACTACGACCCCCC | 380 | RepBm | 14 | 15 |
| | | | AAACGAACACCGCAGGTAATGA | | | | |
| | | | CTAAGA*T*G | | | | |
| oJO437 | RNF2 | 4 A-to-C | GTTATATTATTACCGGCAACGA | 381 | DCV | 14 | 15 |
| | | | ACACCGCAGGTAATGACTAAGA | | | | |
| | | | *T*G | | | | |
| oJO438 | RNF2 | 4 A-to-C | GGCTTAGTATTACCCCCAACGA | 382 | FBYNV | 14 | 15 |
| | | | ACACCGCAGGTAATGACTAAGA | | | | |
| | | | *T*G | | | | |
| oJO442 | RNF2 | 4 A-to-C | CGTTTAATATTACCGGAAACGA | 383 | TGMV | 14 | 15 |
| | | | ACACCGCAGGTAATGACTAAGA | | | | |
| | | | *T*G | | | | |
| oJO441 | RNF2 | 4 A-to-C | ATAATAATATTACGCGCAACGA | 384 | MSMV | 14 | 15 |
| | | | ACACCGCAGGTAATGACTAAGA | | | | |
| | | | *T*G | | | | |
| oJO2354 | DNMT1 | 3-5 | CTGTTAGTATTACCAGCCCGTC | 385 | BDFV/ | 10 | 16 |
| | | delAGG | ACCGTTTCTGGCACCAGGA*A* | | COCV/ | | |
| | | | T | | FiCV/ | | |
| | | | | | GuCV/ | | |
| | | | | | PCV1 | | |
| oJO2355 | DNMT1 | 3-5 | CTGTCAGTATTACCAGCCCGTC | 386 | NG12/ | 10 | 16 |
| | | delAGG | ACCGTTTCTGGCACCAGGA*A* | | NG14/ | | |
| | | | T | | PK5006/ | | |
| | | | | | PK5034/ | | |
| | | | | | PK5222/ | | |
| | | | | | PK5510/ | | |
| | | | | | T25/ | | |
| | | | | | Chimp11 | | |
| oJO2356 | DNMT1 | 3-5 | CTGTTAATACTATCAGCCCGTC | 387 | StCV, | 10 | 16 |
| | | delAGG | ACCGTTTCTGGCACCAGGA*A* | | CaCV, | | |
| | | | T | | Chimp17 | | |
| oJO2357 | DNMT1 | 3-5 | CTGTTATTATTACCAGCCCGTC | 388 | GoCV, | 10 | 16 |
| | | delAGG | ACCGTTTCTGGCACCAGGA*A* | | SwCV | | |
| | | | T | | | | |
| oJO2358 | DNMT1 | 3-5 | CTGTTAGTATTACCAGCCCGTC | 385 | NG13 | 10 | 16 |
| | | delAGG | ACCGTTTCTGGCACCAGGA*A* | | | | |
| | | | T | | | | |
| oJO2359 | DNMT1 | 3-5 | CTGTGAGTATTACCAGCCCGTC | 389 | RaCV | 10 | 16 |
| | | delAGG | ACCGTTTCTGGCACCAGGA*A* | | | | |
| | | | T | | | | |
| oJO2360 | DNMT1 | 3-5 | CTGTGTGTGGGTGTGCAGCCCG | 390 | Cdc13/ | 10 | 16 |
| | | delAGG | TCACCGTTTCTGGCACCAGGA* | | Cdc13 | | |
| | | | A*T | | (Y556A) | | |
| oJO1343 | ACTB | 5 G-to-C | CTGTAAGTATTACCAGCGCGAT | 391 | PCV2 | 19 | 16 |
| | | | ATCATCATCGATGGTGAGCTGC | | | | |
| | | | GAGAAT*A*G | | | | |
| oJO1339 | ACTB | 5 G-to-C | CTGTAAGTATTACCAGCCGATA | 392 | PCV2 | 18 | 14 |
| | | | TCATCATCGATGGTGAGCTGCG | | | | |
| | | | AGA*A*T | | | | |
| oJO1340 | ACTB | 5 G-to-C | CTGTAAGTATTACCAGCGCGAT | 393 | PCV2 | 19 | 14 |
| | | | ATCATCATCGATGGTGAGCTGC | | | | |
| | | | GAGA*A*T | | | | |
| oJO1341 | ACTB | 5 G-to-C | CTGTAAGTATTACCAGCGGCGA | 394 | PCV2 | 20 | 14 |
| | | | TATCATCATCGATGGTGAGCTG | | | | |
| | | | CGAGA*A*T | | | | |
| oJO1342 | ACTB | 5 G-to-C | CTGTAAGTATTACCAGCCGATA | 395 | PCV2 | 18 | 16 |
| | | | TCATCATCGATGGTGAGCTGCG | | | | |
| | | | AGAAT*A*G | | | | |
| oJO1344 | ACTB | 5 G-to-C | CTGTAAGTATTACCAGCGGCGA | 396 | PCV2 | 20 | 16 |
| | | | TATCATCATCGATGGTGAGCTG | | | | |
| | | | CGAGAAT*A*G | | | | |
| oJO1345 | ACTB | 5 G-to-C | CTGTAAGTATTACCAGCCGATA | 397 | PCV2 | 18 | 18 |
| | | | TCATCATCGATGGTGAGCTGCG | | | | |
| | | | AGAATAG*C*C | | | | |
| oJO1346 | ACTB | 5 G-to-C | CTGTAAGTATTACCAGCGCGAT | 398 | PCV2 | 19 | 18 |
| | | | ATCATCATCGATGGTGAGCTGC | | | | |
| | | | GAGAATAG*C*C | | | | |
| oJO1347 | ACTB | 5 G-to-C | CTGTAAGTATTACCAGCGGCGA | 399 | PCV2 | 20 | 18 |
| | | | TATCATCATCGATGGTGAGCTG | | | | |
| | | | CGAGAATAG*C*C | | | | |
| oJO1348 | ACTB | 5 G-to-C | CTGTAAGTATTACCAGCATCAT | 400 | PCV2 | 11 | 20 |
| | | | CGATGGTGAGCTGCGAGAATAG | | | | |
| | | | CC*G*G | | | | |
| oJO1349 | ACTB | 5 G-to-C | CTGTAAGTATTACCAGCATCAT | 401 | PCV2 | 14 | 20 |
| | | | CATCGATGGTGAGCTGCGAGAA | | | | |
| | | | TAGCC*G*G | | | | |
| oJO1350 | ACTB | 5 G-to-C | CTGTAAGTATTACCAGCGATAT | 402 | PCV2 | 17 | 20 |
| | | | CATCATCGATGGTGAGCTGCG | | | | |
| | | | AGAATAGCC*G*G | | | | |
| oJO1351 | ACTB | 5 G-to-C | CTGTAAGTATTACCAGCCGATA | 403 | PCV2 | 18 | 20 |
| | | | TCATCATCGATGGTGAGCTGCG | | | | |
| | | | AGAATAGCC*G*G | | | | |
| oJO1352 | ACTB | 5 G-to-C | CTGTAAGTATTACCAGCGCGAT | 404 | PCV2 | 19 | 20 |
| | | | ATCATCATCGATGGTGAGCTGC | | | | |
| | | | GAGAATAGCC*G*G | | | | |
| oJO1353 | ACTB | 5 G-to-C | CTGTAAGTATTACCAGCGGCGA | 405 | PCV2 | 20 | 20 |
| | | | TATCATCATCGATGGTGAGCTG | | | | |
| | | | CGAGAATAGCC*G*G | | | | |
| oJO2303 | ACTB | 3 nt | CTGTAAGTATTACCAGCGCGAT | 406 | PCV2 | 19 | 16 |
| | | substi- | ATCATCATGGATGTTGAGCTGC | | | | |
| | | tution | GAGAAT*A*G | | | | |
| oJO1264 | DNMT1 | 3-5 | CTGTAAGTATTACCAGCCCGTC | 407 | PCV2 | 10 | 10 |
| | | delAGG | ACCGTTTCTGGCA*C*C | | | | |
| oJO1265 | DNMT1 | 3-5 | CTGTAAGTATTACCAGCCCGTC | 408 | PCV2 | 10 | 12 |
| | | delAGG | ACCGTTTCTGGCACC*A*G | | | | |
| oJO1266 | DNMT1 | 3-5 | CTGTAAGTATTACCAGCCCGTC | 409 | PCV2 | 10 | 14 |
| | | delAGG | ACCGTTTCTGGCACCAG*G*A | | | | |
| oJO1267 | DNMT1 | 3-5 | CTGTAAGTATTACCAGCCCGTC | 410 | PCV2 | 10 | 16 |
| | | delAGG | ACCGTTTCTGGCACCAGGA*A* | | | | |
| | | | T | | | | |
| oJO1268 | DNMT1 | 3-5 | CTGTAAGTATTACCAGCCCGTC | 411 | PCV2 | 10 | 18 |
| | | delAGG | ACCGTTTCTGGCACCAGGAAT* | | | | |
| | | | C*C | | | | |
| oJO1269 | DNMT1 | 3-5 | CTGTAAGTATTACCAGCCCGTC | 412 | PCV2 | 10 | 20 |
| | | delAGG | ACCGTTTCTGGCACCAGGAATC | | | | |
| | | | C*C*C | | | | |
| oJO1270 | DNMT1 | 3-5 | CTGTAAGTATTACCAGCCCCGT | 413 | PCV2 | 11 | 12 |
| | | delAGG | CACCGTTTCTGGCACC*A*G | | | | |
| oJO1271 | DNMT1 | 3-5 | CTGTAAGTATTACCAGCCCCGT | 414 | PCV2 | 11 | 18 |
| | | delAGG | CACCGTTTCTGGCACCAGGAAT | | | | |
| | | | *C*C | | | | |
| oJO1272 | DNMT1 | 3-5 | CTGTAAGTATTACCAGCCTCCC | 415 | PCV2 | 13 | 18 |
| | | | GTCACCGTTTCTGGCACCAGGA | | | | |
| | | delAGG | AT*C*C | | | | |
| oJO1273 | DNMT1 | 3-5 | CTGTAAGTATTACCAGCGCCCT | 416 | PCV2 | 16 | 18 |
| | | delAGG | CCCGTCACCGTTTCTGGCACCA | | | | |
| | | | GGAAT*C*C | | | | |
| oJO1274 | DNMT1 | 3-5 | CTGTAAGTATTACCAGCCTGCC | 417 | PCV2 | 18 | 18 |
| | | delAGG | CTCCCGTCACCGTTTCTGGCAC | | | | |
| | | | CAGGAAT*C*C | | | | |
| oJO1275 | DNMT1 | 3-5 | CTGTAAGTATTACCAGCTCCCG | 418 | PCV2 | 12 | 20 |
| | | delAGG | TCACCGTTTCTGGCACCAGGAA | | | | |
| | | | TCC*C*C | | | | |
| oJO1276 | DNMT1 | 3-5 | CTGTAAGTATTACCAGCGCCCT | 419 | PCV2 | 16 | 20 |
| | | delAGG | CCCGTCACCGTTTCTGGCACCA | | | | |
| | | | GGAATCC*C*C | | | | |
| oJO1277 | DNMT1 | 3-5 | CTGTAAGTATTACCAGCCTGCC | 420 | PCV2 | 18 | 20 |
| | | delAGG | CTCCCGTCACCGTTTCTGGCAC | | | | |
| | | | CAGGAATCC*C*C | | | | |
| oJO2080 | TGFBI | 4 ATins | CTGTAAGTATTACCAGCCTGTA | 421 | PCV2 | 19 | 12 |
| | | | CACGGACCATGCACGGAGAAGC | | | | |
| | | | TG*A*G | | | | |
| oJO2081 | TGFBI | 4 ATins | CTGTAAGTATTACCAGCCTGTA | 422 | PCV2 | 19 | 14 |
| | | | CACGGACCATGCACGGAGAAGC | | | | |
| | | | TGAG*G*C | | | | |
| oJO2082 | TGFBI | 4 ATins | CTGTAAGTATTACCAGCTGTAC | 423 | PCV2 | 18 | 16 |
| | | | ACGGACCATGCACGGAGAAGCT | | | | |
| | | | GAGGC*C*T | | | | |
| oJO2083 | TGFBI | 4 ATins | CTGTAAGTATTACCAGCCTGTA | 424 | PCV2 | 19 | 16 |
| | | | CACGGACCATGCACGGAGAAGC | | | | |
| | | | TGAGGC*C*T | | | | |
| oJO2084 | TGFBI | 4 ATins | CTGTAAGTATTACCAGCGCTGT | 425 | PCV2 | 20 | 16 |
| | | | ACACGGACCATGCACGGAGAAG | | | | |
| | | | CTGAGGC*C*T | | | | |
| oJO2085 | TGFBI | 4 ATins | CTGTAAGTATTACCAGCTGTAC | 426 | PCV2 | 18 | 18 |
| | | | ACGGACCATGCACGGAGAAGCT | | | | |
| | | | GAGGCCT*G*A | | | | |
| oJO2086 | TGFBI | 4 ATins | CTGTAAGTATTACCAGCCTGTA | 427 | PCV2 | 19 | 18 |
| | | | CACGGACCATGCACGGAGAAGC | | | | |
| | | | TGAGGCCT*G*A | | | | |
| oJO2087 | TGFBI | 4 ATins | CTGTAAGTATTACCAGCGCTGT | 428 | PCV2 | 20 | 18 |
| | | | ACACGGACCATGCACGGAGAAG | | | | |
| | | | CTGAGGCCT*G*A | | | | |
| oJO2088 | TGFBI | 4 ATins | CTGTAAGTATTACCAGCTGTAC | 429 | PCV2 | 18 | 20 |
| | | | ACGGACCATGCACGGAGAAGCT | | | | |
| | | | GAGGCCTGA*G*A | | | | |
| oJO2089 | TGFBI | 4 ATins | CTGTAAGTATTACCAGCCTGTA | 430 | PCV2 | 19 | 20 |
| | | | CACGGACCATGCACGGAGAAGC | | | | |
| | | | TGAGGCCTGA*G*A | | | | |
| oJO1894 | IL2RB | 1 T-to-A, | CTGTAAGTATTACCAGCCTCCG | 431 | PCV2 | 9 | 12 |
| | | 5 G-to-C | ACTTCTTTGAAAGA*C*A | | | | |
| oJO1895 | IL2RB | 1 T-to-A, | CTGTAAGTATTACCAGCCTCCG | 432 | PCV2 | 9 | 14 |
| | | 5 G-to-C | ACTTCTTTGAAAGACA*C*C | | | | |
| oJO1896 | IL2RB | 1 T-to-A, | CTGTAAGTATTACCAGCCTCCG | 433 | PCV2 | 9 | 16 |
| | | 5 G-to-C | ACTTCTTTGAAAGACACC*T*G | | | | |
| oJO1897 | IL2RB | 1 T-to-A, | CTGTAAGTATTACCAGCCTCCG | 434 | PCV2 | 9 | 18 |
| | | 5 G-to-C | ACTTCTTTGAAAGACACCTG*G | | | | |
| | | | *A | | | | |
| oJO1898 | IL2RB | 1 T-to-A, | CTGTAAGTATTACCAGCCTCCG | 435 | PCV2 | 9 | 20 |
| | | 5 G-to-C | ACTTCTTTGAAAGACACCTGGA | | | | |
| | | | *G*T | | | | |
| oJO1899 | IL2RB | 1 T-to-A, | CTGTAAGTATTACCAGCCTCCG | 436 | PCV2 | 9 | 22 |
| | | 5 G-to-C | ACTTCTTTGAAAGACACCTGGA | | | | |
| | | | GT*T*C | | | | |
| oJO1900 | IL2RB | 1 T-to-A, | CTGTAAGTATTACCAGCGCCTC | 437 | PCV2 | 11 | 18 |
| | | 5 G-to-C | CGACTTCTTTGAAAGACACCTG | | | | |
| | | | *G*A | | | | |
| oJO1901 | IL2RB | 1 T-to-A, | CTGTAAGTATTACCAGCAGCCT | 438 | PCV2 | 12 | 14 |
| | | 5 G-to-C | CCGACTTCTTTGAAAGACA*C* | | | | |
| | | | C | | | | |
| oJO1902 | IL2RB | 1 T-to-A, | CTGTAAGTATTACCAGCAAGCC | 439 | PCV2 | 13 | 14 |
| | | 5 G-to-C | TCCGACTTCTTTGAAAGACA*C | | | | |
| | | | *C | | | | |
| oJO1903 | IL2RB | 1 T-to-A, | CTGTAAGTATTACCAGCCAAGC | 440 | PCV2 | 14 | 14 |
| | | 5 G-to-C | CTCCGACTTCTTTGAAAGACA* | | | | |
| | | | C*C | | | | |
| oJO1904 | IL2RB | 1 T-to-A, | CTGTAAGTATTACCAGCCCAAG | 441 | PCV2 | 15 | 14 |
| | | 5 G-to-C | CCTCCGACTTCTTTGAAAGACA | | | | |
| | | | *C*C | | | | |
| oJO1905 | IL2RB | 1 T-to-A, | CTGTAAGTATTACCAGCCCAAG | 442 | PCV2 | 15 | 18 |
| | | 5 G-to-C | CCTCCGACTTCTTTGAAAGACA | | | | |
| | | | CCTG*G*A | | | | |
| oJO1906 | IL2RB | 1 T-to-A, | CTGTAAGTATTACCAGCCCAAG | 443 | PCV2 | 15 | 20 |
| | | 5 G-to-C | CCTCCGACTTCTTTGAAAGACA | | | | |
| | | | CCTGGA*G*T | | | | |
| oJO1907 | IL2RB | 1 T-to-A, | CTGTAAGTATTACCAGCCTCCC | 444 | PCV2 | 18 | 16 |
| | | 5 G-to-C | AAGCCTCCGACTTCTTTGAAAG | | | | |
| | | | ACACC*T*G | | | | |
| oJO1908 | IL2RB | 1 T-to-A, | CTGTAAGTATTACCAGCTCTCC | 445 | PCV2 | 19 | 16 |
| | | 5 G-to-C | CAAGCCTCCGACTTCTTTGAAA | | | | |
| | | | GACACC*T*G | | | | |
| oJO1909 | IL2RB | 1 T-to-A, | CTGTAAGTATTACCAGCCCTCC | 446 | PCV2 | 10 | 14 |
| | | 5 G-to-C | GACTTCTTTGAAAGACA*C*C | | | | |
| oJO1878 | PRNP | 6 G-to-T | CTGTAAGTATTACCAGCTGTAG | 447 | PCV2 | 11 | 14 |
| | | | ACGCCAAGGCCCCCCACC*A*C | | | | |
| oJO1879 | PRNP | 6 G-to-T | CTGTAAGTATTACCAGCATGTA | 448 | PCV2 | 12 | 12 |
| | | | GACGCCAAGGCCCCCCA*C*C | | | | |
| oJO1880 | PRNP | 6 G-to-T | CTGTAAGTATTACCAGCATGTA | 449 | PCV2 | 12 | 16 |
| | | | GACGCCAAGGCCCCCCACCAC* | | | | |
| | | | T*G | | | | |
| oJO1881 | PRNP | 6 G-to-T | CTGTAAGTATTACCAGCGCATG | 450 | PCV2 | 14 | 12 |
| | | | TAGACGCCAAGGCCCCCCA*C* | | | | |
| | | | C | | | | |
| oJO1882 | PRNP | 6 G-to-T | CTGTAAGTATTACCAGCAGCAT | 451 | PCV2 | 15 | 12 |
| | | | GTAGACGCCAAGGCCCCCCA*C | | | | |
| | | | *C | | | | |
| oJO1883 | PRNP | 6 G-to-T | CTGTAAGTATTACCAGCCAGCA | 452 | PCV2 | 16 | 12 |
| | | | TGTAGACGCCAAGGCCCCCCA* | | | | |
| | | | C*C | | | | |
| oJO1884 | PRNP | 6 G-to-T | CTGTAAGTATTACCAGCCCAGC | 453 | PCV2 | 17 | 12 |
| | | | ATGTAGACGCCAAGGCCCCCCA | | | | |
| | | | *C*C | | | | |
| oJO1885 | PRNP | 6 G-to-T | CTGTAAGTATTACCAGCCCCAG | 454 | PCV2 | 18 | 12 |
| | | | CATGTAGACGCCAAGGCCCCCC | | | | |
| | | | A*C*C | | | | |
| oJO1886 | PRNP | 6 G-to-T | CTGTAAGTATTACCAGCTCCCA | 455 | PCV2 | 19 | 12 |
| | | | GCATGTAGACGCCAAGGCCCCC | | | | |
| | | | CA*C*C | | | | |
| oJO1887 | PRNP | 6 G-to-T | CTGTAAGTATTACCAGCTTCCC | 456 | PCV2 | 20 | 12 |
| | | | AGCATGTAGACGCCAAGGCCCC | | | | |
| | | | CCA*C*C | | | | |
| oJO1888 | PRNP | 6 G-to-T | CTGTAAGTATTACCAGCCAGCA | 457 | PCV2 | 16 | 14 |
| | | | TGTAGACGCCAAGGCCCCCCAC | | | | |
| | | | C*A*C | | | | |
| oJO1889 | PRNP | 6 G-to-T | CTGTAAGTATTACCAGCTTCCC | 458 | PCV2 | 20 | 14 |
| | | | AGCATGTAGACGCCAAGGCCCC | | | | |
| | | | CCACC*A*C | | | | |
| oJO1890 | PRNP | 6 G-to-T | CTGTAAGTATTACCAGCAGCAT | 459 | PCV2 | 15 | 16 |
| | | | GTAGACGCCAAGGCCCCCCACC | | | | |
| | | | AC*T*G | | | | |
| oJO1891 | PRNP | 6 G-to-T | CTGTAAGTATTACCAGCTCCCA | 460 | PCV2 | 19 | 16 |
| | | | GCATGTAGACGCCAAGGCCCCC | | | | |
| | | | CACCAC*T*G | | | | |
| oJO1892 | PRNP | 6 G-to-T | CTGTAAGTATTACCAGCTTCCC | 461 | PCV2 | 20 | 16 |
| | | | AGCATGTAGACGCCAAGGCCCC | | | | |
| | | | CCACCAC*T*G | | | | |
| oJO1893 | PRNP | 6 G-to-T | CTGTAAGTATTACCAGCCAGCA | 462 | PCV2 | 16 | 22 |
| | | | TGTAGACGCCAAGGCCCCCCAC | | | | |
| | | | CACTGCCCC*A*G | | | | |
| oJO1775 | ACTB | 5 G-to-C, | CTGTAAGTATTACCAGCGCGAT | 463 | PCV2 | 19 | 16 |
| | | 3 A-to-G | ATCATCATCGACGGTGAGCTGC | | | | |
| | | | GAGAAT*A*G | | | | |
| oJO1776 | ACTB | 5 G-to-C, | CTGTAAGTATTACCAGCGCGAT | 464 | PCV2 | 19 | 16 |
| | | 3 A-to-G, | ATCATCATCGACGTTGAGCTGC | | | | |
| | | 1 C-to-A | GAGAAT*A*G | | | | |
| oJO1777 | ACTB | 5 G-to-C, | CTGTAAGTATTACCAGCGCGAT | 465 | PCV2 | 19 | 16 |
| | | 3 A-to-G, | ATCATCATAGACGTTGAGCTGC | | | | |
| | | 1 C-to-A, | GAGAAT*A*G | | | | |
| | | 6 G-to-T | | | | | |
| oJO1778 | ACTB | 5 G-to-C, | CTGTAAGTATTACCAGCGCGAT | 466 | PCV2 | 19 | 16 |
| | | 3 A-to-G, | ATCATCAGAGACGTTGAGCTGC | | | | |
| | | 1 C-to-A, | GAGAAT*A*G | | | | |
| | | 6 G-to-T, | | | | | |
| | | 7 A-to-C | | | | | |
| oJO1780 | ACTB | 5 G-to-C, | CTGTAAGTATTACCAGCGCGAT | 467 | PCV2 | 19 | 16 |
| | | 3 A-to-G, | ATCATGAGAGACGTTGAGCTGC | | | | |
| | | 1 C-to-A, | GAGAAT*A*G | | | | |
| | | 6 G-to-T, | | | | | |
| | | 7 A-to-C, | | | | | |
| | | 9 G-to-C | | | | | |
| oJO1781 | ACTB | 5 G-to-C, | CTGTAAGTATTACCAGCGCGAT | 468 | PCV2 | 19 | 16 |
| | | 3 A-to-G, | ATCATGAGAGCCGTTGAGCTGC | | | | |
| | | 1 C-to-A, | GAGAAT*A*G | | | | |
| | | 6 G-to-T, | | | | | |
| | | 7 A-to-C, | | | | | |
| | | 9 G-to-C, | | | | | |
| | | 2 C-to-G | | | | | |
| oJO2076 | IL2RB | 1 T-to-A, | CTGTAAGTATTACCAGCCAAGC | 469 | PCV2 | 14 | 14 |
| | | 5 G-to-C, | CTCCGATTTCTTTGAAAGACA* | | | | |
| | | 3 G-to-A | C*C | | | | |
| oJO2077 | IL2RB | 1 T-to-A, | CTGTAAGTATTACCAGCCAAGC | 470 | PCV2 | 14 | 14 |
| | | 5 G-to-C, | CTCAGATTTCTTTGAAAGACA* | | | | |
| | | 3 G-to-A | C*C | | | | |
| | | 6 G-to-T | | | | | |
| oJO2078 | IL2RB | 1 T-to-A, | CTGTAAGTATTACCAGCCAAGC | 471 | PCV2 | 14 | 14 |
| | | 5 G-to-C, | CTCTGATTTCTTTGAAAGACA* | | | | |
| | | 3 G-to-A | C*C | | | | |
| | | 6 G-to-C | | | | | |
| oJO2079 | IL2RB | 1 T-to-A, | CTGTAAGTATTACCAGCCAAGC | 472 | PCV2 | 14 | 14 |
| | | 5 G-to-C, | CTCGGATTTCTTTGAAAGACA* | | | | |
| | | 3 G-to-A, | C*C | | | | |
| | | 6 G-to-A | | | | | |
| oJO1967 | GJB2 | 2 delG | CTGTAAGTATTACCAGCAGTGT | 473 | PCV2 | 19 | 20 |
| | | | TTGTTCACACCCCCAGGATCGT | | | | |
| | | | CTGCAGCGTG*C*C | | | | |
| oJO1968 | GJB2 | 2 delG | CTGTAAGTATTACCAGCGTGTT | 474 | PCV2 | 18 | 22 |
| | | | TGTTCACACCCCCAGGATCGTC | | | | |
| | | | TGCAGCGTGCC*C*C | | | | |
| oJO1969 | GJB2 | 2 delG | CTGTAAGTATTACCAGCAGTGT | 475 | PCV2 | 19 | 22 |
| | | | TTGTTCACACCCCCAGGATCGT | | | | |
| | | | CTGCAGCGTGCC*C*C | | | | |
| oJO333 | HEK3 | used as | CTGTAAGTATTACCAGCTCTGC | 476 | PCV2 | 10 | 13 |
| | | non- | GATCACGTGCTCAGTC*T*G | | | | |
| | | targeting | | | | | |
| | | clkDNA | | | | | |
| oMLE57 | EMX1 | 6 G-to-C | CTGTAAGTATTACCAGCAGCAA | 477 | PCV2 | 19 | 14 |
| | | | GCAGCACTGTGCCCTCGTGGGT | | | | |
| | | | TTGT*G*G | | | | |
| oMLE58 | EMX1 | 6 G-to-C | CTGTAAGTATTACCAGCGCAGC | 478 | PCV2 | 14 | 17 |
| | | | ACTGTGCCCTCGTGGGTTTGTG | | | | |
| | | | GT*T*G | | | | |
| oJO2575 | HEK3 | 1 His6 | CTGTAAGTATTACCAGCTGGAG | 479 | PCV2 | 52 | 13 |
| | | (SEQ ID | GAAGCAGGGCTTCCTTTCCTCT | | | | |
| | | NO: 164) | GCCATCAATGATGGTGATGATG | | | | |
| | | ins | GTGCGTGCTCAGTC*T*G | | | | |
| oJO2576 | HEK3 | 1 FLAG | CTGTAAGTATTACCAGCTGGAG | 480 | PCV2 | 58 | 13 |
| | | ins | GAAGCAGGGCTTCCTTTCCTCT | | | | |
| | | | GCCATCACTTATCGTCGTCATC | | | | |
| | | | CTTGTAATCCGTGCTCAGTC*T | | | | |
| | | | *G | | | | |
| 0JO2577 | HEK3 | 1 LoxP | CTGTAAGTATTACCAGCTGGAG | 481 | PCV2 | 74 | 13 |
| | | ins | GAAGCAGGGCTTCCTTTCCTCT | | | | |
| | | | GCCATCAATAACTTCGTATAAT | | | | |
| | | | GTATGCTATACGAAGTTATAAC | | | | |
| | | | AATCGTGCTCAGTC*T*G | | | | |
| oCJT2083 | AAVS1 | 40bp | CTGTAAGTATTACCAGCGATGG | 482 | PCV2 | 57 | 17 |
| | | LoxP | AGCCAGAGAGGAATTGTTATAA | | | | |
| | | ins | CTTCGTATAGCATACATTATAC | | | | |
| | | | GAAGTTATCCCAGAACCTGAGC | | | | |
| | | | *T*G | | | | |
| oCJT2084 | AAVS1 | 40bp | CTGTAAGTATTACCAGCGCAGC | 483 | PCV2 | 57 | 17 |
| | | LoxP | TCAGGTTCTGGGATAACTTCGT | | | | |
| | | ins | ATAATGTATGCTATACGAAGTT | | | | |
| | | | ATAACAATTCCTCTCTGGCTCC | | | | |
| | | | *A*T | | | | |
| oJO1979 | ACTB | attB | CTGTAAGTATTACCAGCTGTCG | 484 | PCV2 | 34 | 16 |
| | | ins | ACGACGGCGGTCTCCGTCGTCA | | | | |
| | | (dual | GGATCATTGAGCTGCGAGAAT* | | | | |
| | | flap) | A*G | | | | |
| oJO1983 | ACTB | attB | TCCTGACGACGGAGACCGCCGT | 485 | n/a | 34 | 16 |
| | | ins | CGTCGACAAGCCGGGCCGTCTT | | | | |
| | | (dual | CCCC*T*C | | | | |
| | | flap) | | | | | |

Optimized clkDNAs with Additional End Modifications
(2′oME, hairpins, hairpins, 5′biotin)

| oligo | target | | | SEQ ID | | recuit- |
|---|---|---|---|---|---|---|
| ID | gene | edit | clkDNA sequence | NO(s): | modification | ment |
| oJO2407 | ACTB | 5 G- | CTGTAAGTATTACCAGCGCGATATCATCATCGA | 486 | 4x 3′PS | PCV2 |
| | | to-C | TGGTGAGCTGCGAGA*A*T*A*G | | | |
| oJ)2562 | ACTB | 5 G- | CTGTAAGTATTACCAGCGCGATATCATCATCGA | 487 | 3x PBS | PCV2 |
| | | to-C | TGGAGACCTCCGAGAAT*A*G | | mutations | |
| oCJT2136 | ACTB | 5 G- | CTGTAAGTATTACCAGCGCGATATCATCATCGA | 488 | 3x 3′PS/2′- | PCV2 |
| | | to-C | TGGTGAGCTGCGAGAA*mU*mA*mG | | O-Me | |
| oJO2595 | ACTB | 5 G- | C*T*G*TAAGTATTACCAGCGCGATATCATCAT | 489 | 3x 5′PS | PCV2 |
| | | to-C | CGATGGTGAGCTGCGAGAAT*A*G | | | |
| oJO2586 | ACTB | 5 G | mC*mU*mG*TAAGTATTACCAGCGCGATATCAT | 490 | 3x | PCV2 |
| | | to-C | CATCGATGGTGAGCTGCGAGAAT*A*G | | 5′PBS/2′oME | |
| oCRC174 | ACTB | 5 G- | CTGTAAGTATTACCAGCGCGATATCATCATCGA | 491 | 3′ evopreQ1 | PCV2 |
| | | to-C | TGGTGAGCTGCGAGAATAGAATAAACCCGCGGT | | | |
| | | | TCTATCTAGTTACGCGTTAAACCAACTAGAA | | | |
| oJO2408 | DNMT1 | 3-5 | CTGTAAGTATTACCAGCCCGTCACCGTTTCTGG | 492 | 4x 3′PS | PCV2 |
| | | AGG | CACCAG*G*A*A*T | | | |
| | | del | | | | |
| oJO2563 | DNMT1 | 3-5 | CTGTAAGTATTACCAGCCCGTCACCGTATCAGG | 493 | 3x PBS | PCV2 |
| | | AGG | GACCAGGA*A*T | | mutations | |
| | | del | | | | |
| oJO2560 | DNMT1 | 3-5 | CTGTAAGTATTACCAGCCCGTCACCGTTTCTGG | 494 | 3x 3′PS/2′- | PCV2 |
| | | AGG | CACCAGG*mA*mA*mU | | O-Me | |
| | | del | | | | |
| oJO2520 | ACTB | 5 G | /5BiosG/GCGATATCATCATCGATGGTGAGCT | 495 | 5′ biotin | mSA |
| | | to-C | GCGAGAAT*A*G | | | |
| oJO2521 | DNMT1 | 3-5 | /5BiosG/CCGTCACCGTTTCTGGCACCAGGA* | 496 | 5′ biotin | mSA |
| | | AGG | A*T | | | |
| | | del | | | | |
| oCIT2033 | ACTB | 3nt | CTGTAAGTATTACCAGCGCGATATCATCATGGA | 497 | 3x3′PS/2′-O- | PCV2 |
| | | substi- | TGTTGAGCTGCGAGAA*mU*mA*mG | | Me | |
| | | tution | | | | |
| oCRC113 | ACTB | 5 G- | CTGTAAGTATTACCAGCGCGATATCATCATCGA | 498 | 8 nt GAAA | PCV2 |
| | | to-C | TGGTGAGCTGCGAGAATAGGAAACTATTCTCCG | | hairpin + 2 | |
| | | | | | nt non- | |
| | | | | | complemen- | |
| | | | | | tary tail | |
| oCRC114 | ACTB | 5 G | CTGTAAGTATTACCAGCGCGATATCATCATCGA | 499 | 10 nt GAAA | PCV2 |
| | | to-C | TGGTGAGCTGCGAGAATAGGAAACTATTCTCGC | | (SEQ ID NO: | |
| | | | TC | | 708) | |
| | | | | | hairpin + | |
| | | | | | 2 nt non- | |
| | | | | | complemen- | |
| | | | | | tary tail | |
| oCRC115 | ACTB | 5 G | CTGTAAGTATTACCAGCGCGATATCATCATCGA | 500 | 12 nt GAAA | PCV2 |
| | | to-C | TGGTGAGCTGCGAGAATAGGAAACTATTCTCGC | | (SEQ ID NO: | |
| | | | AGGA | | 710) | |
| | | | | | hairpin + | |
| | | | | | 2 nt non- | |
| | | | | | complemen- | |
| | | | | | tary tail | |
| oCRC116 | ACTB | 5 G | CTGTAAGTATTACCAGCGCGATATCATCATCGA | 501 | 14 nt GAAA | PCV2 |
| | | to-C | TGGTGAGCTGCGAGAATAGGAAACTATTCTCGC | | (SEQ ID NO: | |
| | | | AGCTGT | | 712) | |
| | | | | | hairpin + | |
| | | | | | 2 nt non- | |
| | | | | | complemen- | |
| | | | | | tary tail | |
| oCRC117 | ACTB | 5 G- | CTGTAAGTATTACCAGCGCGATATCATCATCGA | 502 | 16 nt GAAA | PCV2 |
| | | to-C | TGGTGAGCTGCGAGAATAGGAAACTATTCTCGC | | (SEQ ID NO: | |
| | | | AGCTCAGG | | 713) | |
| | | | | | hairpin + | |
| | | | | | 2 nt non- | |
| | | | | | complemen- | |
| | | | | | tary tail | |
| oCRC118 | ACTB | 5 G- | CTGTAAGTATTACCAGCGCGATATCATCATCGA | 503 | 18 nt GAAA | PCV2 |
| | | to-C | TGGTGAGCTGCGAGAATAGGAAACTATTCTCGC | | (SEQ ID NO: | |
| | | | AGCTCACCTA | | 714) | |
| | | | | | hairpin + | |
| | | | | | 2 nt non- | |
| | | | | | complemen- | |
| | | | | | tary tail | |
| oCRC119 | ACTB | 5 G- | CTGTAAGTATTACCAGCGCGATATCATCATCGA | 504 | 8 nt GAAA | PCV2 |
| | | to-C | TGGTGAGCTGCGAGAATAGCCAGGTCCGAAAGG | | stem loop + | |
| | | | ACCTGGGA | | 2 nt non- | |
| | | | | | complemen | |
| | | | | | tary tail | |
| oCRC120 | ACTB | 5 G | CTGTAAGTATTACCAGCGCGATATCATCATCGA | 505 | 10 nt GAAA | PCV2 |
| | | to-C | TGGTGAGCTGCGAGAATAGCCAGGTCCAGGAAA | | (SEQ ID NO: | |
| | | | CTGGACCTGGGA | | 708) stem | |
| | | | | | loop + 2 nt | |
| | | | | | non- | |
| | | | | | complemen- | |
| | | | | | tary tail | |
| oCRC121 | ACTB | 5 G- | CTGTAAGTATTACCAGCGCGATATCATCATCGA | 506 | 11 nt GAAA | PCV2 |
| | | to-C | TGGTGAGCTGCGAGAATAGCCAGGTCCAGGGAA | | (SEQ ID NO: | |
| | | | ACCTGGACCTGGGA | | 709) stem | |
| | | | | | loop + 2 nt | |
| | | | | | non- | |
| | | | | | complemen- | |
| | | | | | tary tail | |
| oCRC122 | ACTB | 5 G- | CTGTAAGTATTACCAGCGCGATATCATCATCGA | 507 | 12 nt GAAA | PCV2 |
| | | to-C | TGGTGAGCTGCGAGAATAGCCAGGTCCAGGTGA | | (SEQ ID NO: | |
| | | | AAACCTGGACCTGGGA | | 710) stem | |
| | | | | | loop + 2 nt | |
| | | | | | non- | |
| | | | | | complemen- | |
| | | | | | tary tail | |
| oCRC123 | ACTB | 5 G- | CTGTAAGTATTACCAGCGCGATATCATCATCGA | 508 | 13 nt GAAA | PCV2 |
| | | to-C | TGGTGAGCTGCGAGAATAGCCAGGTCCAGGTCG | | (SEQ ID NO: | |
| | | | AAAGACCTGGACCTGGGA | | 711) stem | |
| | | | | | loop + 2 nt | |
| | | | | | non- | |
| | | | | | complemen- | |
| | | | | | tary tail | |
| oCRC124 | ACTB | 5 G- | CTGTAAGTATTACCAGCGCGATATCATCATCGA | 509 | 14 nt GAAA | PCV2 |
| | | to-C | TGGTGAGCTGCGAGAATAGCCAGGTCCAGGTCC | | (SEQ ID NO: | |
| | | | GAAAGGACCTGGACCTGGGA | | 712) stem | |
| | | | | | loop + 2 nt | |
| | | | | | non- | |
| | | | | | complemen- | |
| | | | | | tary tail | |
| oCRC150 | ACTB | 5 G- | CTGTAAGTATTACCAGCGCGATATCATCATCGA | 510 | 10 nt GAAA | PCV2 |
| | | to-C | TGGTGAGCTGCGAGAATAGGAAACTATTCTCGC | | (SEQ ID NO: | |
| | | | | | 708) hairpin | |
| | | | | | (un- | |
| | | | | | protected) | |
| oCRC151 | ACTB | 5 G- | CTGTAAGTATTACCAGCGCGATATCATCATCGA | 511 | 16 nt GAAA | PCV2 |
| | | to-C | TGGTGAGCTGCGAGAATAGGAAACTATTCTCGC | | (SEQ ID NO: | |
| | | | AGCTCA | | 713) hairpin | |
| | | | | | (un- | |
| | | | | | protected) | |
| oCRC152 | ACTB | 5 G- | CTGTAAGTATTACCAGCGCGATATCATCATCGA | 512 | 10 nt GAAA | PCV2 |
| | | to-C | TGGTGAGCTGCGAGAATAGCCAGGTCCAGGAAA | | (SEQ ID NO: | |
| | | | CTGGACCTGG | | 708) stem | |
| | | | | | loop (un- | |
| | | | | | protected) | |
| oCRC153 | ACTB | 5 G- | CTGTAAGTATTACCAGCGCGATATCATCATCGA | 513 | 16 nt GAAA | PCV2 |
| | | to-C | TGGTGAGCTGCGAGAATAGCCAGGTCCAGGTCG | | (SEQ ID NO: | |
| | | | AAAGACCTGGACCTGG | | 713) stem | |
| | | | | | loop (un- | |
| | | | | | protected) | |
| oCRC101 | DNMT1 | 3-5 | CTGTAAGTATTACCAGCCCGTCACCGTTTCTGG | 514 | 8 nt GAAA | PCV2 |
| | | AGG | CACCAGGAATGAAAATTCCTGGAC | | hairpin + | |
| | | del | | | 2 nt non- | |
| | | | | | complemen- | |
| | | | | | tary tail | |
| oCRC102 | DNMT1 | 3-5 | CTGTAAGTATTACCAGCCCGTCACCGTTTCTGG | 515 | 10 nt GAAA | PCV2 |
| | | AGG | CACCAGGAATGAAAATTCCTGGTGGG | | (SEQ ID NO: | |
| | | del | | | 708) | |
| | | | | | hairpin + | |
| | | | | | 2 nt non- | |
| | | | | | complemen- | |
| | | | | | tary tail | |
| oCRC103 | DNMT1 | 3-5 | CTGTAAGTATTACCAGCCCGTCACCGTTTCTGG | 516 | 12 nt GAAA | PCV2 |
| | | AGG | CACCAGGAATGAAAATTCCTGGTGCCTC | | (SEQ ID NO: | |
| | | del | | | 710) | |
| | | | | | hairpin + | |
| | | | | | 2 nt non- | |
| | | | | | complemen- | |
| | | | | | tary tail | |
| oCRC104 | DNMT1 | 3-5 | CTGTAAGTATTACCAGCCCGTCACCGTTTCTGG | 517 | 14 nt GAAA | PCV2 |
| | | AGG | CACCAGGAATGAAAATTCCTGGTGCCAGTT | | (SEQ ID NO: | |
| | | del | | | 712) | |
| oCRC105 | DNMT1 | 3-5 | CTGTAAGTATTACCAGCCCGTCACCGTTTCTGG | 518 | 16 nt GAAA | PCV2 |
| | | AGG | CACCAGGAATGAAAATTCCTGGTGCCAGAATG | | (SEQ ID NO: | |
| | | del | | | 713) | |
| | | | | | hairpin + | |
| | | | | | 2 nt non- | |
| | | | | | complemen- | |
| | | | | | tary tail | |
| oCRC106 | DNMT1 | 3-5 | CTGTAAGTATTACCAGCCCGTCACCGTTTCTGG | 519 | 18 nt GAAA | PCV2 |
| | | AGG | CACCAGGAATGAAAATTCCTGGTGCCAGAAACC | | (SEQ ID NO: | |
| | | del | C | | 714) | |
| | | | | | hairpin + | |
| | | | | | 2 nt non- | |
| | | | | | complemen- | |
| | | | | | tary tail | |
| oCRC107 | DNMT1 | 3-5 | CTGTAAGTATTACCAGCCCGTCACCGTTTCTGG | 520 | 8 nt GAAA | PCV2 |
| | | AGG | CACCAGGAATCCAGGTCCGAAAGGACCTGGTA | | stem loop + | |
| | | del | | | 2 nt non- | |
| | | | | | complemen- | |
| | | | | | tary tail | |
| oCRC108 | DNMT1 | 3-5 | CTGTAAGTATTACCAGCCCGTCACCGTTTCTGG | 521 | 10 nt GAAA | PCV2 |
| | | AGG | CACCAGGAATCCAGGTCCAGGAAACTGGACCTG | | (SEQ ID NO: | |
| | | del | GTA | | 708) stem | |
| | | | | | loop + 2 nt | |
| | | | | | non- | |
| | | | | | complemen- | |
| | | | | | tary tail | |
| oCRC109 | DNMT1 | 3-5 | CTGTAAGTATTACCAGCCCGTCACCGTTTCTGG | 522 | 11 nt GAAA | PCV2 |
| | | AGG | CACCAGGAATCCAGGTCCAGGGAAACCTGGACC | | (SEQ ID NO: | |
| | | del | TGGTA | | 709) stem | |
| | | | | | loop + 2 nt | |
| | | | | | non- | |
| | | | | | complemen- | |
| | | | | | tary tail | |
| oCRC110 | DNMT1 | 3-5 | CTGTAAGTATTACCAGCCCGTCACCGTTTCTGG | 523 | 12 nt GAAA | PCV2 |
| | | AGG | CACCAGGAATCCAGGTCCAGGTGAAAACCTGGA | | (SEQ ID NO: | |
| | | del | CCTGGTA | | 710) stem | |
| | | | | | loop + 2 nt | |
| | | | | | non- | |
| | | | | | complemen- | |
| | | | | | tary tail | |
| oCRC111 | DNMT1 | 3-5 | CTGTAAGTATTACCAGCCCGTCACCGTTTCTGG | 524 | 13 nt GAAA | PCV2 |
| | | AGG | CACCAGGAATCCAGGTCCAGGTCGAAAGACCTG | | (SEQ ID NO: | |
| | | del | GACCTGGTA | | 711) stem | |
| | | | | | loop + 2 nt | |
| | | | | | non- | |
| | | | | | complemen- | |
| | | | | | tary tail | |
| oCRC112 | DNMT1 | 3-5 | CTGTAAGTATTACCAGCCCGTCACCGTTTCTGG | 525 | 14 nt | PCV2 |
| | | AGG | CACCAGGAATCCAGGTCCAGGTCCGAAAGGACC | | GAAA(SEQ | |
| | | del | TGGACCTGGTA | | ID NO: 712) | |
| | | | | | stem loop + | |
| | | | | | 2 nt non- | |
| | | | | | complemen- | |
| | | | | | tary tail | |
| oCRC144 | DNMT1 | 3-5 | CTGTAAGTATTACCAGCCCGTCACCGTTTCTGG | 526 | 10 nt GAAA | PCV2 |
| | | AGG | CACCAGGAATGAAAATTCCTGGTG | | (SEQ ID NO: | |
| | | del | | | 708) | |
| | | | | | hairpin | |
| | | | | | (un- | |
| | | | | | protected) | |
| oCRC145 | DNMT1 | 3-5 | CTGTAAGTATTACCAGCCCGTCACCGTTTCTGG | 527 | 16 nt GAAA | PCV2 |
| | | AGG | CACCAGGAATGAAAATTCCTGGTGCCAGAA | | (SEQ ID NO: | |
| | | del | | | 713) hairpin | |
| | | | | | (un- | |
| | | | | | protected) | |
| oCRC146 | DNMT1 | 3-5 | CTGTAAGTATTACCAGCCCGTCACCGTTTCTGG | 528 | 10 nt GAAA | PCV2 |
| | | AGG | CACCAGGAATCCAGGTCCAGGAAACTGGACCTG | | (SEQ ID NO: | |
| | | del | G | | 708) stem | |
| | | | | | loop (un- | |
| | | | | | protected) | |
| oCRC147 | DNMT1 | 3-5 | CTGTAAGTATTACCAGCCCGTCACCGTTTCTGG | 529 | 16 nt GAAA | PCV2 |
| | | AGG | CACCAGGAATCCAGGTCCAGGTCGAAAGACCTG | | (SEQ ID NO: | |
| | | del | GACCTGG | | 713) stem | |
| | | | | | loop (un- | |
| | | | | | protected) | |
| oCRC89 | RNF2 | 4 A- | CTGTAAGTATTACCAGCAACGAACACCGCAGGT | 530 | 8 nt GAAA | PCV2 |
| | | to-C | AATGACTAAGATGACGAAAGTCATCTTTC | | hairpin + 2 | |
| | | | | | nt non- | |
| | | | | | complemen- | |
| | | | | | tary tail | |
| oCRC90 | RNF2 | 4 A- | CTGTAAGTATTACCAGCAACGAACACCGCAGGT | 531 | 10 nt GAAA | PCV2 |
| | | to-C | AATGACTAAGATGACGAAAGTCATCTTAGAG | | (SEQ ID NO: | |
| | | | | | 708) | |
| | | | | | hairpin + | |
| | | | | | 2 nt non- | |
| | | | | | complemen- | |
| | | | | | tary tail | |
| oCRC91 | RNF2 | 4 A- | CTGTAAGTATTACCAGCAACGAACACCGCAGGT | 532 | 12 nt GAAA | PCV2 |
| | | to-C | AATGACTAAGATGACGAAAGTCATCTTAGTCTA | | (SEQ ID NO: | |
| | | | | | 710) | |
| | | | | | hairpin + | |
| | | | | | 2 nt non- | |
| | | | | | complemen- | |
| | | | | | tary tail | |
| oCRC92 | RNF2 | 4 A- | CTGTAAGTATTACCAGCAACGAACACCGCAGGT | 533 | 14 nt GAAA | PCV2 |
| | | to-C | AATGACTAAGATGACGAAAGTCATCTTAGTCAT | | (SEQ ID NO: | |
| | | | AT | | 712) | |
| | | | | | hairpin + | |
| | | | | | 2 nt non- | |
| | | | | | complemen- | |
| | | | | | tary tail | |
| oCRC93 | RNF2 | 4 A- | CTGTAAGTATTACCAGCAACGAACACCGCAGGT | 534 | 16 nt GAAA | PCV2 |
| | | to-C | AATGACTAAGATGACGAAAGTCATCTTAGTCAT | | (SEQ ID NO: | |
| | | | TAGG | | 713) | |
| | | | | | hairpin + | |
| | | | | | 2 nt non- | |
| | | | | | complemen- | |
| | | | | | tary tail | |
| oCRC94 | RNF2 | 4 A- | CTGTAAGTATTACCAGCAACGAACACCGCAGGT | 535 | 18 nt GAAA | PCV2 |
| | | to-C | AATGACTAAGATGACGAAAGTCATCTTAGTCAT | | (SEQ ID NO: | |
| | | | TACCAC | | 714) | |
| | | | | | hairpin + | |
| | | | | | 2 nt non- | |
| | | | | | complemen- | |
| | | | | | tary tail | |
| oCRC95 | RNF2 | 4 A- | CTGTAAGTATTACCAGCAACGAACACCGCAGGT | 536 | 8 nt GAAA | PCV2 |
| | | to-C | AATGACTAAGATGACCCAGGTCCGAAAGGACCT | | stem loop + | |
| | | | GGCA | | 2 nt non- | |
| | | | | | complemen- | |
| | | | | | tary tail | |
| 0CRC96 | RNF2 | 4 A- | CTGTAAGTATTACCAGCAACGAACACCGCAGGT | 537 | 10 nt GAAA | PCV2 |
| | | to-C | AATGACTAAGATGACCCAGGTCCAGGAAACTGG | | (SEQ ID NO: | |
| | | | ACCTGGCA | | 708) stem | |
| | | | | | loop + 2 nt | |
| | | | | | non- | |
| | | | | | complemen | |
| | | | | | tary tail | |
| oCRC97 | RNF2 | 4 A- | CTGTAAGTATTACCAGCAACGAACACCGCAGGT | 538 | 11 nt GAAA | PCV2 |
| | | to-C | AATGACTAAGATGACCCAGGTCCAGGGAAACCT | | (SEQ ID NO: | |
| | | | GGACCTGGCA | | 709) stem | |
| | | | | | loop + 2 nt | |
| | | | | | non- | |
| | | | | | complemen- | |
| | | | | | tary tail | |
| oCRC98 | RNF2 | 4 A- | CTGTAAGTATTACCAGCAACGAACACCGCAGGT | 539 | 12 nt GAAA | PCV2 |
| | | to-C | AATGACTAAGATGACCCAGGTCCAGGTGAAAAC | | (SEQ ID NO: | |
| | | | CTGGACCTGGCA | | 710) stem | |
| | | | | | loop + 2 nt | |
| | | | | | non- | |
| | | | | | complemen- | |
| | | | | | tary tail | |
| oCRC99 | RNF2 | 4 A- | CTGTAAGTATTACCAGCAACGAACACCGCAGGT | 540 | 13 nt GAAA | PCV2 |
| | | to-C | AATGACTAAGATGACCCAGGTCCAGGTCGAAAG | | (SEQ ID NO: | |
| | | | ACCTGGACCTGGCA | | 711) stem | |
| | | | | | loop + 2 nt | |
| | | | | | non- | |
| | | | | | complemen- | |
| | | | | | tary tail | |
| oCRC100 | RNF2 | 4 A- | CTGTAAGTATTACCAGCAACGAACACCGCAGGT | 541 | 14 nt GAAA | PCV2 |
| | | to-C | AATGACTAAGATGACCCAGGTCCAGGTCCGAAA | | (SEQ ID NO: | |
| | | | GGACCTGGACCTGGCA | | 712) stem | |
| | | | | | loop + 2 nt | |
| | | | | | non- | |
| | | | | | complemen- | |
| | | | | | tary tail | |
| oCRC137 | RNF2 | 4 A- | CTGTAAGTATTACCAGCAACGAACACCGCAGGT | 542 | 10 nt GAAA | PCV2 |
| | | to-C | AATGACTAAGATGACGAAAGTCATCTTAG | | (SEQ ID NO: | |
| | | | | | 708) hairpin | |
| | | | | | (un- | |
| | | | | | protected) | |
| oCRC138 | RNF2 | 4 A- | CTGTAAGTATTACCAGCAACGAACACCGCAGGT | 543 | 16 nt GAAA | PCV2 |
| | | to-C | AATGACTAAGATGACGAAAGTCATCTTAGTCAT | | (SEQ ID NO: | |
| | | | TA | | 713) hairpin | |
| | | | | | (un- | |
| | | | | | protected) | |
| oCRC139 | RNF2 | 4 A- | CTGTAAGTATTACCAGCAACGAACACCGCAGGT | 544 | 10 nt GAAA | PCV2 |
| | | to-C | AATGACTAAGATGACCCAGGTCCAGGAAACTGG | | (SEQ ID NO: | |
| | | | ACCTGG | | 708) stem | |
| | | | | | loop (un- | |
| | | | | | protected) | |
| oCRC140 | RNF2 | 4 A- | CTGTAAGTATTACCAGCAACGAACACCGCAGGT | 545 | 16 nt GAAA | PCV2 |
| | | to-C | AATGACTAAGATGACCCAGGTCCAGGTCGAAAG | | (SEQ ID NO: | |
| | | | ACCTGGACCTGG | | 713) stem | |
| | | | | | loop (un- | |
| | | | | | protected) | |
| oJO2517 | ACTB | 5 G | rGrCrArCrArUrGrArGrGrArUrCrArCrCr | 546 | 5′ MS2 stem | PCV2 |
| | | to C | CrArUrGrUrGrCGCGATATCATCATCGATGGT | | loop (un- | |
| | | | GAGCTGCGAGAAT*A*G | | protected) | |
| oJO2514 | ACTB | 5 G | mG*mC*mA*rCrArUrGrArGrGrArUrCrArC | 547 | 5′ MS2 stem | PCV2 |
| | | to C | rCrCrArUrGrUrGrCGCGATATCATCATCGAT | | loop | |
| | | | GGTGAGCTGCGAGAAT*A*G | | (protected) | |
| oJO2518 | DNMT1 | 3-5 | rGrCrArCrArUrGrArGrGrArUrCrArCrCr | 548 | 5′ MS2 stem | PCV2 |
| | | AGG | CrArUrGrUrGrCCCGTCACCGTTTCTGGCACC | | loop (un- | |
| | | del | AGGA*A*T | | protected) | |
| oJO2515 | DNMT1 | 3-5 | mG*mC*mA*rCrArUrGrArGrGrArUrCrArC | 549 | 5′ MS2 stem | PCV2 |
| | | AGG | rCrCrArUrGrUrGrCCCGTCACCGTTTCTGGC | | loop | |
| | | del | ACCAGGA*A*T | | (protected) | |

bold/underlined = HUHe/TBP recognition sequence. Extra 5′ sequence added to ensure efficient HUHe/TBP binding to clkDNA (sequences from Lovendahl et al., J. Am. Chem. Soc. 2017 and Li et al. Journal of Virology 2010)

TABLE 5
HUH pseudosites, identified using TagScan

| | SEQ ID | | FIGs. |
|---|---|---|---|
| Context | NO(S): | Original | 36B-D ID |
| AAGTATTACCAGCAAAGG | 550 | NC_000006.11[99191761 . . . 99191778] | Site 1 |
| AAGTATTACCAGCCCAGG | 551 | NC_000001.10[55506595 . . . 55506612] | |
| AAGTATTACCAGCATAGG | 552 | NC_000006.11[109987010 . . . 109987027] | Site 2 |
| AAGTATTACCAGCCTGGG | 553 | NC_000001.10[8847951 . . . 8847968] | Site 3 |
| AAGTATTACCAGCTTTGG | 554 | NC_000016.9[11128128 . . . 11128145] | Site 4 |
| AAGTATTACCAGCAAGG | 555 | NC_000002.11[1367494 . . . 1367510] | Site 5 |
| AAGTATTACCAGCCAGG | 556 | NC_000003.11[69863983 . . . 69863999] | Site 6 |
| AAGTATTACCAGCCTGG | 557 | NC_000001.10[8847951 . . . 8847967] | |
| AAGTATTACCAGCCTGG | 557 | NC_000009.11[73587679 . . . 73587695] | Site 7 |
| AAGTATTACCAGCCCTAGGG | 558 | NC_000003.11[193799404 . . . 193799423] | Site 8 |
| AAGTATTACCAGCAGAATGG | 257 | NC_000011.9[84267991 . . . 84268010] | Site 9 |
| AAGTATTACCAGCAACCTGG | 559 | NC_000010.10[57632781 . . . 57632800] | |
| AAGTATTACCAGCATCCTGG | 560 | NC_000001.10[215599270 . . . 215599289] | Site 10 |
| AAGTATTACCAGCTAATTGG | 561 | NC_000019.9[44412076 . . . 44412095] | |
| AAGTATTACCAGCTAATTGG | 561 | NC_000019.9[44412119 . . . 44412138] | |
| AAGTATTACCAGCTATTTGG | 562 | NC_000023.10[100286297 . . . 100286316] | Site 11 |
| AAGTATTACCAGCCCTAGG | 563 | NC_000003.11[193799404 . . . 193799422] | Site 12 |
| AAGTATTACCAGCCGTAGG | 564 | NC_000012.11[122864906 . . . 122864924] | Site 13 |
| AAGTATTACCAGCATAGGG | 565 | NC_000006.11[109987009 . . . 109987027] | |
| AAGTATTACCAGCAACAGTCGG | 566 | NC_000010.10[57306021 . . . 57306042] | |
| AAGTATTACCAGCAATTATTGG | 567 | NC_000002.11[201318184 . . . 201318205] | |
| AAGTATTACCAGCAAGAAAGAGG | 568 | NC_000016.9[16115093 . . . 16115115] | Site 14 |
| AAGTATTACCAGCAGAATGGAGG | 569 | NC_000011.9[84267991 . . . 84268013] | Site 15 |
| AAGTATTACCAGCCCTTCATCGG | 570 | NC_000012.11[50127697 . . . 50127719] | Site 16 |
| AAGTATTACCAGCAATTATTGGG | 571 | NC_000002.11[201318183 . . . 201318205] | |

TABLE 6
Sequences to construct HUHe phylogeny
ID/Name

>UniRef50_W1I5Y6 Uncultured bacterium plasmid pRGF00066 n = 1 Tax = uncultured bacterium
TaxID = 77133 RepID = W1I5Y6_9BACT
>pdb|3DKX|A Chain A, Replication protein repB
>UniRef100_A0A1Y4QQC0 Replication protein RepB n = 1 Tax = Enterococcus cecorum TaxID = 44008
RepID = A0A1Y4QQC0_9ENTE
>UniRef100_A0A174GG61 Replication protein repB n = 1 Tax = Collinsella aerofaciens TaxID = 74426
RepID = A0A174GG61_9ACTN
>UniRef50_W1I697 Uncultured bacterium plasmid pRGI00782 n = 1 Tax = uncultured bacterium
TaxID = 77133 RepID = W1I697_9BACT
>UniRef100_A0A1C6AUS2 Replication protein repB n = 1 Tax = uncultured Ruminococcus sp.
TaxID = 165186 RepID = A0A1C6AUS2_9FIRM
>UniRef50_G8CNT2 Putative replication protein n = 2 Tax = Bacteria TaxID = 2 RepID = G8CNT2_9BACT
>UniRef50_A0A0H5PZI7 Replication protein n = 1 Tax = uncultured prokaryote TaxID = 198431
RepID = A0A0H5PZI7_9ZZZZ
>UniRef50_W1I557 Uncultured bacterium plasmid pRGI00247 n = 1 Tax = uncultured bacterium
TaxID = 77133 RepID = W1I557_9BACT
>UniRef100_U2EU97 OrfA n = 8 Tax = Bacteria TaxID = 2 RepID = U2EU97_9BACT
>UniRef50_A0A0H5Q0X0 Plasmid replication protein n = 1 Tax = uncultured prokaryote TaxID = 198431
RepID = A0A0H5Q0X0_9ZZZZ
>UniRef50_K9RZV9 Plasmid replication initiation protein n = 5 Tax = Mycoplasma TaxID = 2093
RepID = K9RZV9_MYCCA
>UniRef50_A0A1W6BZG0 Replication protein n = 2 Tax = Campylobacterales TaxID = 213849
RepID = A0A1W6BZG0_9BACT
>UniRef50_A0A0R3QHC2 Replication protein n = 1 Tax = Brugia timori TaxID = 42155
RepID = A0A0R3QHC2_9BILA
>UniRef50_A0A0H5PZW4 Plasmid replication protein n = 1 Tax = uncultured prokaryote TaxID = 198431
RepID = A0A0H5PZW4_9ZZZZ
>UniRef50_A0A1Y4G0S9 Replication protein RepB n = 1 Tax = Gordonibacter sp. An230 TaxID = 1965592
RepID = A0A1Y4G0S9_9ACTN
>UniRef100_UPI000300949E replication protein n = 1 Tax = Streptococcus agalactiae TaxID = 1311
RepID = UPI000300949E
>UniRef50_A0A0H5PV05 Replication protein n = 1 Tax = uncultured prokaryote TaxID = 198431
RepID = A0A0H5PV05_9ZZZZ
>UniRef50_W7D2V3 RepB n = 4 Tax = Bacilli TaxID = 91061 RepID = W7D2V3_9LIST
>UniRef100_A0A0H5PZG0 Replication protein n = 1 Tax = uncultured prokaryote TaxID = 198431
RepID = A0A0H5PZG0_9ZZZZ
>UniRef50_A0A0H5QIL6 Replication protein n = 2 Tax = uncultured prokaryote TaxID = 198431
RepID = A0A0H5QIL6_9ZZZZ
>UniRef50_A0A0H5PZA0 Plasmid replication protein n = 3 RepID = A0A0H5PZA0_9ZZZZ
>UniRef50_A0A0Z8IYX5 Plasmid replication protein n = 16 Tax = Bacilli TaxID = 91061
RepID = A0A0Z8IYX5_STRSU
>UniRef100_A0A087EKU7 Replication protein RepB n = 2 Tax = Bifidobacterium tsurumiense
TaxID = 356829 RepID = A0A087EKU7_9BIFI
>UniRef100_A0A0S2MGE2 Plasmid replication protein n = 2 Tax = Bifidobacterium TaxID = 1678
RepID = A0A0S2MGE2_BIFLN
>UniRef50_U2QZX4 Plasmid replication protein n = 17 Tax = Bacillota TaxID = 1239 RepID = U2QZX4_9FIRM
>UniRef50_A0A0H5Q8X5 Plasmid replication protein n = 1 Tax = uncultured prokaryote TaxID = 198431
RepID = A0A0H5Q8X5_9ZZZZ
>UniRef100_F0HMF3 Plasmid replication protein n = 11 Tax = Terrabacteria group TaxID = 1783272
RepID = F0HMF3_9ACTN
>UniRef50_U2TJ01 Plasmid replication protein (Fragment) n = 1 Tax = Olsenella profusa F0195
TaxID = 1125712 RepID = U2TJ01_9ACTN
>UniRef50_A0A1Y3UDM3 Plasmid replication protein n = 1 Tax = Collinsella sp. An7 TaxID = 1965651
RepID = A0A1Y3UDM3_9ACTN
>UniRef50_A0A1B1IHL4 Replication protein n = 4 Tax = Streptococcus TaxID = 1301
RepID = A0A1B1IHL4_9STRE
>UniRef50_K7YFJ8 RepH n = 4 Tax = Helicobacter pylori TaxID = 210 RepID = K7YFJ8_HELPX
>UniRef50_O31070 Putative Rep protein n = 3 Tax = Helicobacter pylori TaxID = 210 RepID = O31070_HELPX
>UniRef100_S6F6F1 Plasmid replication protein repB n = 1 Tax = Lactococcus lactis subsp. lactis A12
TaxID = 1137134 RepID = S6F6F1_LACLL
>UniRef50_K9RYD5 Plasmid replication initiation protein n = 6 Tax = Mycoplasma mycoides group
TaxID = 656088 RepID = K9RYD5_MYCCA
>UniRef50_A0A0R1P770 Rep50 n = 4 Tax = Limosilactobacillus mucosae TaxID = 97478
RepID = A0A0R1P770_LIMMU
>UniRef50_Q48831 RepB protein n = 6 Tax = Lactobacillies TaxID = 186826 RepID = Q48831_LACPN
>ADN84041.1 replication associated protein [Rhynchosia golden mosaic Havana virus-
[Cuba:Havana:28:2007]]
>AAN76737.1 replication-associated protein AC1 [Macroptilium mosaic Puerto Rico virus-[Bean]]
>AAB87607.1 replication-associated protein [Cowpea golden mosaic virus-[Nigeria]]
>AGK24653.1 C1 [Soybean chlorotic blotch virus]
>ABG90906.1 C1 [Merremia leaf curl virus]
>ACY79450.1 Rep [Sweet potato mosaic virus - [Brazil:Brasilia1:2007]]
>YP_001040016.1 replication associated protein [Tomato leaf curl Seychelles virus]
>YP_002224032.1 AC1 [Jatropha leaf curl virus]
>ACB44970.1 replication-associated protein [Tomato leaf curl Palampur virus-[Roodan:tomato]]
>AGJ03640.1 replicase [Chilli leaf curl Bijnour virus-India]
>CBJ17676.1 replication initiator protein [Croton yellow vein mosaic virus]
>AMK07575.1 AC1 protein [Lycianthes yellow mosaic virus]
>AFA26437.2 replication-associated protein [Pedilanthus leaf curl virus-[Crape Jasmine]]
>CAJ85998.1 AC1 protein [Malvastrum leaf curl Guangdong virus]
>AGF41094.1 C1 [Papaya leaf curl virus]
>AHL29198.1 AC1 protein [Sida yellow vein Vietnam virus]
>YP_764516.1 replication-associated protein [Okra yellow crinkle virus]
>AGG08895.1 AC1 [Lindernia anagallis yellow vein virus]
>CDW92215.1 Replication Associate Protein [Tomato leaf curl Mali virus]
>NP_620741.1 Rep protein [Tomato yellow leaf curl Sardinia virus]
>YP_006905839.1 C1 [Jatropha mosaic Nigeria virus]
>BAF02752.1 Replication initiation protein (Rep) [Pepper yellow leaf curl Indonesia virus-[Ageratum]]
>AFF58888.1 AC1 [Clerodendrum golden mosaic China virus]
>AAX39336.1 replication-associated protein [African cassava mosaic virus]
>AGV02071.1 AC1 protein [Okra leaf curl virus-[India:Mau:2012]]
>AAF75542.1 AC1 [Tobacco curly shoot virus - [Y1]]
>ACV60535.1 AC1 protein [Tomato leaf curl Vietnam virus]
>NP_050017.1 AC1 replicase-associated protein [Pepper leaf curl virus]
>AFB81519.1 replication associated protein [Cotton leaf curl Rajasthan virus]
>ACI06063.1 replication initiator protein [Mesta yellow vein mosaic virus [India:Amadalavalasa:2008]]
>AFB83419.1 replication associated protein [Cotton leaf curl Burewala virus]
>AFH68197.1 replication-associated protein [Hollyhock yellow vein mosaic virus]
>ABD35287.1 AC1 [Alternanthera yellow vein virus]
>AEY63664.1 Rep [Watermelon chlorotic stunt virus]
>YP_003622552.1 replication-associated protein [Soybean mild mottle virus]
>ADW24243.1 Rep protein [Kudzu mosaic virus]
>AKS48121.1 replication initiator protein [Rhynchosia yellow mosaic virus]
>YP_009129272.1 C1 protein [Apple geminivirus 1]
>CAM91896.1 replication associated protein [Tomato leaf curl Anjouan virus]
>AJM13604.1 replication-associated protein [Asystasia mosaic Madagascar virus]
>YP_004958233.1 replication associated protein [West African Asystasia virus 1]
>AIY31184.1 AC1 protein [Tomato yellow leaf curl virus]
>AHA82274.1 AC1 [Tomato leaf curl New Delhi virus 4]
>YP_001333687.1 rep protein [Corchorus golden mosaic virus]
>YP_115511.1 rep protein [Corchorus yellow vein virus - [Hoa Binh]]
>YP_001285764.1 rep protein [Spilanthes yellow vein virus]
>AMP46444.1 replication initiator protein [Deinbollia mosaic virus]
>YP_008411025.1 AC1 [Hemidesmus yellow mosaic virus]
>AAP73446.1 replication initiation protein AC1 [Dolichos yellow mosaic virus]
>NP_671468.1 replication-associated protein [Macroptilium yellow mosaic Florida virus]
>AMW86999.1 replication associated protein [Sida golden yellow vein virus]
>AGH29892.1 replication-associated protein [Tomato leaf distortion virus]
>CBH28932.1 replication-associated protein [Sida micrantha mosaic virus]
>CBA18089.1 replication associated protein [Sida golden mosaic Brazil virus]
>ALV85583.1 replication-associated protein [Pavonia mosaic virus]
>AFD54490.1 replication-associated protein [Macroptilium yellow spot virus]
>YP_002941855.1 replication-associated protein [Passionfruit severe leaf distortion virus]
>YP_006590064.1 replication-associated protein [Soybean chlorotic spot virus]
>ACV83312.1 AC1 [Tomato yellow vein streak virus]
>AER09339.1 replication-associated protein [Cleome leaf crumple virus]
>YP_007250561.1 replication-associated protein [Tomato yellow mottle virus]
>YP_619883.1 replication protein [Tomato golden mottle virus]
>AHX57826.1 replication-associated protein [Jacquemontia yellow mosaic virus]
>NP_040557.1 replication-associated protein [Beet curly top virus]
>ALF37659.1 rolling circle replication initiator protein [Beet curly top virus]
>YP_003778178.1 Rep [Turnip curly top virus]
>YP_009226627.1 Rep [Turnip leaf roll virus]
>ALR86823.1 Rep [Turnip leaf roll virus]
>ABD67440.1 Rep [Mungbean yellow mosaic India virus]
>AEE99005.1 C1 protein [Tomato yellow leaf curl virus - KISR]
>APP87725.1 Rep/C1 [Grapevine geminivirus A]
>AGV02076.1 Rep [Cotton chlorotic spot virus]
>AAL96826.1 replication associated protein AC1 [Cotton leaf crumple virus - [TX]]
>YP_003828907.1 AC1 [Melon chlorotic mosaic virus]
>NP_066185.1 rep [Horseradish curly top virus]
>YP_003966137.1 replication initiator protein [Spinach severe curly top virus]
>AFM38721.1 replication initiator protein [Jacquemontia mosaic Yucatan virus]
>CRI68211.1 Replication protein [Pepper golden mosaic virus]
>ACO88014.1 replication associated protein [Eragrostis curvula streak virus]
>AFV91331.1 replication-associated protein [Dragonfly-associated mastrevirus]
>YP_006666531.1 Rep [Sporobolus striate mosaic virus 2]
>AIT39773.1 replication-associated protein RepB [Switchgrass mosaic-associated virus]
>YP_006666527.1 Rep [Sporobolus striate mosaic virus 1]
>AAK73446.1 Rep [Maize streak virus - A[MatB]]
>AHM88378.1 replication associated protein [Sugarcane streak Reunion virus]
>sp|Q80GM6.2|REP_SSVN RecName: Full = Replication-associated protein; Short = Rep
>AHM88382.1 replication associated protein [Urochloa streak virus]
>YP_003288768.1 RepA [Saccharum streak virus]
>sp|P0C647.1|REP_PASVK RecName: Full = Replication-associated protein; Short = Rep
>AHM88370.1 replication associated protein [Maize streak Reunion virus]
>YP_006273070.1 replicase [Wheat dwarf India virus]
>YP_009026388.1 replication associated protein [Sugarcane white streak virus]
>YP_003915159.1 replication associated protein [Digitaria didactyla striate mosaic virus]
>AFN80669.1 Rep [Chloris striate mosaic virus]
>AFN80601.1 Rep [Paspalum striate mosaic virus]
>YP_004089627.1 Rep [Bromus catharticus striate mosaic virus]
>YP_006666523.1 Rep [Paspalum dilatatum striate mosaic virus]
>YP_006666535.1 Rep [Digitaria ciliaris striate mosaic virus]
>YP_009021763.1 replication associated protein [Axonopus compressus streak virus]
>YP_009115514.1 replication-associated protein [Sewage-associated gemycircularvirus 6]
>YP_003104796.1 replication-associated protein [Sclerotinia sclerotiorum hypovirulence associated
DNA virus 1]
>YP_009252368.1 replication associated protein [Faeces associated gemycircularvirus 22]
>YP_009021043.1 replication-associated protein [Cassava associated gemycircularvirus 1]
>YP_009115515.1 replication-associated protein [Sewage-associated gemycircularvirus-7a]
>YP_009115519.1 replication-associated protein [Sewage-associated gemycircularvirus 9]
>AIF34843.1 replication-associated protein [Sewage-associated gemycircularvirus-7b]
>YP_009252356.1 replication associated protein [Lama associated gemycircularvirus 1]
>YP_009252353.1 replication associated protein [Faeces associated gemycircularvirus 20]
>AMH87666.1 replication-associated protein [Pacific flying fox faeces associated gemycircularvirus-2]
>AGS12486.1 replication-associated protein [hypericum associated gemycircularvirus 1]
>AMH87733.1 replication-associated protein [Pteropus associated gemycircularvirus 4]
>AMH87708.1 replication-associated protein [Pteropus associated gemycircularvirus 8]
>YP_009252365.1 replication associated protein [Faeces associated gemycircularvirus 19]
>YP_009252359.1 replication associated protein [Faeces associated gemycircularvirus 18]
>AMH87678.1 replication-associated protein [Pacific flying fox faeces associated gemycircularvirus-4]
>YP_009109733.1 replication associated protein [Gerygone associated gemycircularvirus 3]
>YP_009181999.1 replication associated protein [Soybean associated gemycircularvirus 1]
>YP_009109727.1 replication associated protein [Gerygone associated gemycircularvirus 1]
>YP_009252362.1 replication associated protein [Faeces associated gemycircularvirus 16]
>AMH87702.1 replication-associated protein [Pacific flying fox faeces associated gemycircularvirus-10]
>YP_009109725.1 replication associated protein [Mallard associated gemycircularvirus 1]
>YP_009164036.1 replication-associated protein [Gemycircularvirus gemy-ch-rat1]
>AJD07464.1 replication-associated protein [Odonata associated gemycircularvirus 2]
>AMH87693.1 replication-associated protein [Pteropus associated gemycircularvirus 5]
>YP_009109729.1 replication associated protein [Gerygone associated gemycircularvirus 2]
>AUM61807.1 Rep [uncultured virus]
>YP_009351871.1 putative rep protein [Phomopsis longicolla circular virus 1]
>YP_007008175.1 replication-associated protein (plasmid) [Wheat blue dwarf phytoplasma]
>WP_015083745.1 replication-associated protein [Candidatus Phytoplasma tritici]
>ATL14544.1 replication associated protein (plasmid) [Rice orange leaf phytoplasma]
>YP_001708784.1 replication-associated protein (plasmid) [Paulownia witches'-broom phytoplasma]
>YP_001965310.1 replication associated protein (plasmid) [Candidatus Phytoplasma australiense]
>YP_001708790.1 replication-associated protein (plasmid) [Paulownia witches'-broom phytoplasma]
>YP_007008179.1 replication-associated protein (plasmid) [Wheat blue dwarf phytoplasma]
>WP_013747472.1 replication-associated protein [Chinaberry witches'-broom phytoplasma]
>WP_011412958.1 hypothetical protein [Aster yellows witches'-broom phytoplasma]
>ABC65268.1 conserved hypothetical protein [Aster yellows witches'-broom phytoplasma AYWB]
>YP_006961991.1 replication-associated protein (plasmid) [Periwinkle little leaf phytoplasma]
>WP_011412950.1 hypothetical protein [Aster yellows witches'-broom phytoplasma]
>CBX25033.1 Rep protein [New Jersey aster yellows phytoplasma]
>YP_001965305.1 replication associated protein (plasmid) [Candidatus Phytoplasma australiense]
>WP_017193171.1 hypothetical protein [Italian clover phyllody phytoplasma]
>WP_017193695.1 hypothetical protein [Vaccinium witches'-broom phytoplasma]
>ABC65385.1 conserved hypothetical protein [Aster yellows witches'-broom phytoplasma AYWB]
>YP_006959585.1 replication protein (plasmid) [Onion yellows phytoplasma]
>WP_015060110.1 replication protein [Onion yellows phytoplasma]
>WP_011264167.1 replication protein [Onion yellows phytoplasma]
>BAD36752.1 replication associated protein [Onion yellows phytoplasma]
>WP_042068233.1 hypothetical protein [‘Chrysanthemum coronarium’ phytoplasma]
>WP_012662291.1 replication protein [Onion yellows phytoplasma]
>KXT29032.1 geminivirus Rep catalytic domain protein [Candidatus Phytoplasma oryzae]
>KXT29014.1 geminivirus Rep catalytic domain protein [Candidatus Phytoplasma oryzae]
>AJD07486.1 replication-associated protein [Odonata-associated circular virus-15]
>OLY79419.1 Replication-associated protein [Smittium mucronatum]
>OLY79389.1 Replication-associated protein [Smittium mucronatum]
>OMJ21113.1 Replication-associated protein [Smittium culicis]
>OMJ28371.1 Replication-associated protein A [Smittium culicis]
>OMJ13215.1 Replication-associated protein A, partial [Smittium culicis]
>OMJ11569.1 Replication-associated protein, partial [Smittium culicis]
>OMJ09562.1 Replication-associated protein, partial [Smittium culicis]
>ETO15557.1 rep [Reticulomyxa filosa]
>AUM61713.1 Rep [uncultured virus]
>AUM61719.1 Rep [uncultured virus]
>AUM61624.1 Rep [uncultured virus]
>AUM62043.1 Rep [uncultured virus]
>AUM61738.1 Rep [uncultured virus]
>OLY79699.1 hypothetical protein AYI68_g6225 [Smittium mucronatum]
>AAF36424.1 replicase [Pyropia pulchra]
>AAF36423.1 replicase [Pyropia pulchra]
>AAF36422.1 replicase [Pyropia pulchra]
>YP_007974237.1 replicase [Bat associated circovirus 2]
>AAZ78351.1 rep protein [Porcine circovirus 2]
>AIF76280.1 Rep [Bat associated circovirus 9]
>AGL09969.1 relicase [Bat associated circovirus 1]
>YP_009021891.1 replication-associated protein [Mink circovirus]
>AKO84203.1 replicase [Fox circovirus]
>AIF76265.1 Rep [Bat associated circovirus 5]
>AIF76253.1 Rep [Bat circovirus]
>AIF76248.1 Rep [Bat associated circovirus 8]
>AIF76261.1 Rep [Bat associated circovirus 7]
>YP_009170674.1 replicase [Bat associated circovirus 4]
>ADD62475.1 replication-association protein [Human stool-associated circular virus NG13]
>YP_004376332.1 putative replication protein [Barbel circovirus]
>YP_009091696.1 replication-associated protein [European catfish circovirus]
>YP_009000900.1 replication-associated protein [Anguilla anguilla circovirus]
>AFL02442.1 replication associated protein [Beak and feather disease virus]
>ADU77009.1 Rep protein [Circovirus NGchicken38/NGA/2009]
>YP_764455.1 rep protein [Raven circovirus]
>YP_009134739.1 replicase [Zebra finch circovirus]
>NP_573442.1 replicase [Canary circovirus]
>YP_803546.1 replication-associated protein [Gull circovirus]
>AEL28794.1 replication-associated protein [Bat circovirus ZS/Yunnan-China/2009]
>ABU48445.1 putative replication-associated protein [Swan circovirus]
>AHK80894.1 replication protein [Duck circovirus]
>ADD62451.1 replication-association protein [Cyclovirus PK5006]
>ADD62455.1 replication-association protein [Cyclovirus PK5222]
>ADU77011.1 Rep protein [Cyclovirus NGchicken8/NGA/2009]
>AGJ74758.1 replication-associated protein [Dragonfly associated cyclovirus 7]
>ADD62461.1 replication-association protein [Cyclovirus Chimp11]
>AKE49355.1 replication-associated protein [Cyclovirus NI-204]
>AGJ74756.1 replication-associated protein [Dragonfly associated cyclovirus 6]
>YP_009110680.1 capsid [Bat associated cyclovirus 12]
>AGJ74760.1 replication-associated protein [Dragonfly associated cyclovirus 8]
>YP_009021843.1 replication-associated protein [Dragonfly associated cyclovirus 5]
>AEL87786.1 putative replication-associated protein [Bat associated cyclovirus 2]
>YP_004152331.1 Rep protein [Cyclovirus bat/USA/2009]
>ADD62471.1 replication-association protein [Cyclovirus NG12]
>AIF76252.1 Rep [Bat associated cyclovirus 8]
>AEL87790.1 putative replication-associated protein [Bat associated cyclovirus 3]
>ADI48251.1 putative Rep [Bat cyclovirus GF-4c]
>ADD62453.1 replication-association protein [Cyclovirus PK5034]
>ADY17982.1 replication associated protein [Dragonfly associated cyclovirus 1]
>ADD62457.1 replication-association protein [Cyclovirus PK5510]
>AIF76266.1 Rep [Bat associated cyclovirus 13]
>ADD62473.1 replication-association protein [Cyclovirus NG14]
>AEL87792.1 putative replication-associated protein [Bat faeces associated cyclovirus 4]
>AIF76249.1 Rep [Bat associated cyclovirus 6]
>AIF76254.1 Rep [Bat associated cyclovirus 11]
>YP_009021870.1 replication-associated protein [Human cyclovirus]
>AFS65290.1 replication-associated protein, partial [Dragonfly cyclovirus 2]
>YP_009047065.1 replication-association protein [Cyclovirus VN]
>ADD62477.1 replication-association protein [Cyclovirus TN25]
>YP_008130363.1 replication association protein [Human cyclovirus VS5700009]
>ADU76993.1 Rep protein [Cyclovirus PKbeef23/PAK/2009]
>YP_009237526.1 replication associated protein [Lake Sarah-associated circular virus-14]
>YP_009116910.1 replication-associated protein [Sewage-associated circular DNA virus-23]
>AMH87650.1 replication-associated protein [Pacific flying fox associated cyclovirus-1]
>AMH87652.1 replication-associated protein [Pacific flying fox associated cyclovirus-2]
>AIF76251.1 Rep [Bat circovirus]
>CUO57637.1 Uncharacterised protein [Fusicatenibacter saccharivorans]
>CUO23215.1 Uncharacterised protein [Coprococcus comes]
>CDF01935.1 putative uncharacterized protein [Ruminococcus sp. CAG:624]
>WP_026669310.1 hypothetical protein [Butyrivibrio sp. AE3006]
>WP_026524352.1 hypothetical protein [Butyrivibrio sp. MB2005]
>WP_053982727.1 hypothetical protein [Niameybacter massiliensis]
>WP_003102166.1 hypothetical protein [Streptococcus iniae]
>WP_029176105.1 replication protein RepA [Streptococcus suis]
>WP_000032131.1 hypothetical protein [Streptococcus oralis]
>WP_062004798.1 hypothetical protein [Streptococcus cristatus]
>WP_003030931.1 hypothetical protein [Streptococcus anginosus]
>WP_047207334.1 replication protein RepA [Streptococcus agalactiae]
>WP_029690610.1 hypothetical protein [Streptococcus hyovaginalis]
>WP_029694263.1 hypothetical protein [Streptococcus suis]
>CVH76026.1 hypothetical protein BN3658_00547 [Coriobacteriaceae bacterium CHKCI002]
>SCH60086.1 Putative viral replication protein [uncultured Ruminococcus sp.]
>WP_036328238.1 hypothetical protein [Candidatus Izimaplasma sp. HR2]
>WP_044942941.1 viral replication protein [Flavonifractor plautii]
>WP_021629801.1 hypothetical protein [Flavonifractor plautii]
>CCZ45692.1 putative uncharacterized protein [Firmicutes bacterium CAG:129]
>CBL15233.1 Putative viral replication protein [Ruminococcus bromii L2-63]
>WP_021882760.1 viral replication protein [Ruminococcus sp. CAG:108]
>WP_013978550.1 hypothetical protein [Eggerthella sp. YY7918]
>WP_051600858.1 hypothetical protein [Eubacterium sp. AB3007]
>WP_009301216.1 hypothetical protein [Thomasclavelia ramosa]
>WP_038350939.1 replication protein [Eubacterium limosum]
>KJZ87129.1 hypothetical protein ClosIBUN125C_CONTIG36g01985 [Clostridium sp. IBUN125C]
>WP_023977019.1 replication protein [Clostridium beijerinckii]
>WP_018597672.1 hypothetical protein [Blautia producta]
>CUP05665.1 Viral replication-associated protein [Hungatella hathewayi]
>SCH17786.1 Putative viral replication protein [uncultured Clostridium sp.]
>CDC44519.1 putative replication protein [Clostridium sp. CAG:58]
>BAK32345.1 putative replication protein [Erysipelothrix rhusiopathiae str. Fujisawa]
>WP_044928503.1 replication protein [Roseburia inulinivorans]
>WP_037404274.1 hypothetical protein [Solobacterium moorei]
>CDE72464.1 putative replication protein [Acidaminococcus sp. CAG:917]
>WP_053167095.1 hypothetical protein [Planococcus glaciei]
>CDB27189.1 putative replication protein [Oscillibacter sp. CAG:241]
>WP_009246639.1 hypothetical protein [Blautia hansenii]
>CCY69022.1 putative uncharacterized protein [Eubacterium sp. CAG:161]
>WP_052011064.1 hypothetical protein [bacterium LF-3]
>CCX75435.1 putative uncharacterized protein [Dorea sp. CAG:105]
>WP_024346025.1 hypothetical protein [[Clostridium] methoxybenzovorans]
>WP_038278663.1 hypothetical protein [Lacrimispora celerecrescens]
>WP_007865724.1 MULTISPECIES: hypothetical protein [Clostridia]
>WP_013270924.1 Viral replication-associated protein [Lacrimispora saccharolytica]
>WP_066550639.1 hypothetical protein [Clostridium sp. AT4]
>WP_020072285.1 hypothetical protein [Faecalispora sporosphaeroides]
>WP_051639324.1 hypothetical protein [Lachnospiraceae bacterium AC2028]
>EES75484.2 hypothetical protein RSAG_03452 [Ruminococcus sp. 5_1_39BFAA]
>WP_051546484.1 hypothetical protein [[Clostridium] viride]
>WP_066546553.1 hypothetical protein [Clostridium sp. AT4]
>WP_013271491.1 hypothetical protein [Lacrimispora saccharolytica]
>YP_007517186.1 replication-associated protein [Gastropod associated circular ssDNA virus]
>YP_009126903.1 replication-associated protein [Avon-Heathcote Estuary associated circular virus 12]
>WP_055838650.1 hypothetical protein [Microbacterium sp. Leaf436]
>WP_016667133.1 hypothetical protein [Cutibacterium avidum]
>WP_002529618.1 MULTISPECIES: hypothetical protein [Actinomycetia]
>WP_036342632.1 hypothetical protein, partial [Mobiluncus mulieris]
>NP_613078.1 putative replication protein RepA (plasmid) [Bifidobacterium pseudocatenulatum]
>WP_021975256.1 MULTISPECIES: replication protein RepA [Bifidobacterium]
>KFI81686.1 putative replication protein RepA [Bifidobacterium pullorum]
>WP_043170238.1 hypothetical protein [Bifidobacterium pullorum]
>WP_052825216.1 hypothetical protein [Bifidobacterium animalis]
>KFI87454.1 putative replication protein RepA [Bifidobacterium ruminantium]
>WP_022856850.1 replication-associated family protein [Alloscardovia omnicolens]
>WP_023022037.1 MULTISPECIES: hypothetical protein [unclassified Corynebacterium]
>AKO38848.1 hypothetical protein RZ62_08290 [[Haemophilus] ducreyi]
>WP_052119337.1 hypothetical protein [Bifidobacterium callitrichos]
>WP_025221073.1 hypothetical protein [Bifidobacterium breve]
>WP_033495900.1 hypothetical protein [Bifidobacterium biavatii]
>YP_009163936.1 putative replication initiation protein [Palaemonetes sp. common grass shrimp
associated circular virus]
>AHH31482.1 replication-associated protein [Dragonfly larvae associated circular virus-10]
>YP_009021888.1 replication associated protein [Cyanoramphus nest associated circular X DNA virus]
>YP_009237559.1 replication associated protein [Lake Sarah-associated circular virus-11]
>YP_003104737.1 master replication initiator protein [Faba bean necrotic stunt virus]
>AKO71308.1 Replication associated protein [Banana bunchy top virus]
>AIF34798.1 replication-associated protein [Sewage-associated circular DNA virus-10]
>AAA51422.1 V1 [Banana bunchy top virus]
>ACB86656.1 replication initiation protein [Banana bunchy top virus]
>AAA51426.1 V2 [Banana bunchy top alphasatellite 1]
>YP_009058890.1 orf1 [Faba bean necrotic yellows C1 alphasatellite]
>NP_619760.1 viral replication-associated protein [Milk vetch dwarf C2 alphasatellite]
>NP_619759.1 viral replication-associated protein [Milk vetch dwarf C1 alphasatellite]
>YP_008169853.1 Rep [Cuban alphasatellite 1]
>YP_009246456.1 replication associated protein [Cucurbit yellow mosaic alphasatellite]
>ALK03646.1 replication initiator protein [Whitefly associated Guatemala alphasatellite 2]
>AIY31250.1 putative replicase protein [Camel associated porprismacovirus 1]
>YP_009030025.1 replication associated protein [PoSCV Kor J481]
>YP_009054985.1 Rep protein [Porcine associated porprismacovirus 5]
>YP_009022025.1 replicase [Turkey associated porprismacovirus 1]
>AMR73073.1 replication associated protein [Human feces smacovirus 3]
>YP_009252320.1 replication associated protein [Porcine associated porprismacovirus 10]
>YP_009054987.1 Rep protein [Porcine associated porprismacovirus 7]
>YP_009118276.1 rep protein [Gorilla associated porprismacovirus 1]
>AIY31246.1 putative replicase protein [Camel associated porprismacovirus 2]
>YP_009163761.1 rep protein [Rat associated porprismacovirus 1]
>YP_009054993.1 Rep protein [Porcine associated porprismacovirus 6]
>AMR73071.1 replication associated protein [Human feces smacovirus 2]
>ADB24799.1 putative replicase protein [Chimpanzee associated porprismacovirus 2]
>YP_009118278.1 rep protein [Lemur associated porprismacovirus 1]
>YP_009252308.1 replication associated protein [Sheep faeces associated smacovirus 3]
>YP_009252310.1 replication associated protein [Sheep faeces associated smacovirus 1]
>YP_009252314.1 replication associated protein [Bovine faeces associated smacovirus 3]
>AEW47007.1 putative replicase [Circoviridae bovine stool/BK/KOR/2011]
>YP_009252316.1 replication associated protein [Bovine faeces associated smacovirus 1]
>AJF23062.1 rep protein [Human smacovirus 1]
>AJF23060.1 rep protein [Human associated huchismacovirus 3]
>AJE25847.1 Rep [Human smacovirus 1]
>AJE25851.1 Rep [Human smacovirus 1]
>AJE25845.1 Rep [Human smacovirus 1]
>YP_009252326.1 replication associated protein [Bovine faeces associated smacovirus 6]
>AJD07511.1 replication-associated protein [Odonata-associated circular virus 5]
>AIY31243.1 putative replicase protein [Camel associated drosmacovirus 1]
>AIY31256.1 putative replicase protein [Camel associated drosmacovirus 2]
>WP_034704841.1 Rep family protein [Enterococcus gallinarum]
>WP_067483596.1 MULTISPECIES: Rep family protein [Enterococcus]
>WP_049499636.1 Rep family protein [Streptococcus parasanguinis]
>WP_045759092.1 Rep family protein [Streptococcus parasanguinis]
>KXT86702.1 Replication protein [Streptococcus oralis]
>WP_014623544.1 Rep family protein [Streptococcus equi]
>WP_032497992.1 Rep family protein [Streptococcus suis]
>KEQ49321.1 RNA helicase family protein [Streptococcus oralis]
>WP_053092713.1 Rep family protein [Streptococcus oralis]
>WP_027972054.1 Rep family protein [Streptococcus plurextorum]
>WP_049476139.1 MULTISPECIES: Rep family protein [Streptococcus anginosus group]
>WP_003035134.1 Rep family protein [Streptococcus anginosus]
>WP_017649267.1 Rep family protein [Streptococcus agalactiae]
>ABJ73998.1 Plasmid replication protein Rep and AAA-class ATPase domain [Lactococcus cremoris
subsp. cremoris SK11]
>WP_015647385.1 Rep family protein [Streptococcus suis]
>CGE81062.1 plasmid replication protein Rep and AAA-class ATPase domain protein [Streptococcus
pneumoniae]
>WP_039677656.1 Rep family protein [Streptococcus constellatus]
>WP_044774450.1 Rep family protein [Streptococcus suis]
>WP_020997784.1 Rep family protein [Streptococcus constellatus]
>WP_003024533.1 Rep family protein [Streptococcus anginosus]
>ADX23728.1 plasmid replication protein Rep and AAA-class ATPase domain protein [Streptococcus
dysgalactiae subsp. equisimilis ATCC 12394]
>WP_056938517.1 Rep family protein [Paucilactobacillus suebicus]
>WP_000201649.1 MULTISPECIES: Rep family protein [unclassified Streptococcus]
>WP_047206721.1 MULTISPECIES: Rep family protein [Streptococcus]
>CMU27730.1 plasmid replication protein Rep and AAA-class ATPase domain protein [Streptococcus
pneumoniae]
>WP_000044268.1 Rep family protein [Streptococcus sp. SK140]
>WP_001034312.1 Rep family protein [Streptococcus agalactiae]
>WP_024385235.1 Rep family protein [Streptococcus suis]
>WP_004183001.1 MULTISPECIES: Rep family protein [Streptococcus]
>WP_024400359.1 Rep family protein [Streptococcus suis]
>WP_000791389.1 Rep family protein [Streptococcus sp. oral taxon 071]
>WP_003032217.1 Rep family protein [Streptococcus anginosus]
>WP_018376545.1 Rep family protein [Streptococcus orisratti]
>WP_020999261.1 Rep family protein [Streptococcus intermedius]
>WP_044762265.1 Rep family protein [Streptococcus suis]
>WP_054952722.1 Rep family protein [Flaviflexus massiliensis]
>WP_041290927.1 Rep family protein [Kytococcus sedentarius]
>WP_036321578.1 Rep family protein [Microbacterium gubbeenense]
>WP_051176704.1 Rep family protein [Propionimicrobium lymphophilum]
>WP_052506726.1 Rep family protein [Streptococcus suis]
>WP_022765681.1 Rep family protein [Butyrivibrio sp. XPD2006]
>AEU41945.1 plasmid replication protein (plasmid) [Lactococcus cremoris subsp. cremoris A76]
>WP_025016923.1 Rep family protein [Lactococcus lactis]
>KST89836.1 Replication protein [Lactococcus lactis subsp. lactis]
>WP_018030886.1 MULTISPECIES: Rep family protein [Streptococcus]
>WP_003104234.1 Rep family protein [Streptococcus parauberis]
>WP_039670385.1 Rep family protein [Streptococcus macedonicus]
>WP_018380019.1 Rep family protein [Streptococcus thoraltensis]
>EOB33201.1 plasmid replication protein [Streptococcus mitis 13/39]
>WP_044671103.1 Rep family protein [Streptococcus suis]
>WP_003048523.1 Rep family protein [Streptococcus canis]
>WP_039694464.1 Rep family protein [Streptococcus gallolyticus]
>WP_000746010.1 MULTISPECIES: Rep family protein [Streptococcus]
>WP_019299400.1 Rep family protein [Lactococcus garvieae]
>BAM66968.1 replication protein [Lactococcus lactis]
>WP_032941943.1 Rep family protein, partial [Lactococcus lactis]
>WP_058223604.1 Rep family protein [Lactococcus lactis]
>WP_061343647.1 Rep family protein [Enterococcus faecium]
>WP_017371219.1 MULTISPECIES: Rep family protein [Lactococcus]
>WP_014571792.1 MULTISPECIES: Rep family protein [Lactococcus]
>CDE19587.1 replication protein [Acidiphilium sp. CAG:727]
>CCZ68460.1 putative uncharacterized protein [Mediterraneibacter gnavus CAG:126]
>SCG87263.1 Plasmid replication protein [uncultured Clostridium sp.]
>YP_006961027.1 replication protein (plasmid) [Periwinkle leaf yellowing phytoplasma]
>YP_003617079.1 Rep (plasmid) [‘Rehmannia glutinosa’ phytoplasma]
>ABC65805.1 replication protein (plasmid) [Aster yellows witches'-broom phytoplasma AYWB]
>WP_011161011.1 Rep family protein [Onion yellows phytoplasma]
>ABC65794.1 replication protein (plasmid) [Aster yellows witches'-broom phytoplasma AYWB]
>YP_001966814.1 Rep (plasmid) [Candidatus Phytoplasma australiense]
>KXT29039.1 plasmid replication family protein [Candidatus Phytoplasma oryzae]
>ODR34583.1 hypothetical protein BEI60_22705 [Eisenbergiella tayi]
>CCZ93342.1 aTP-dependent protease Clp ATPase subunit [Coprococcus eutactus CAG:665]
>CCY61699.1 rep [Clostridium sp. CAG:264]
>WP_019282500.1 Rep family protein [Vibrio anguillarum]
>WP_002578150.1 Rep family protein [Enterocloster bolteae]
>CUN62864.1 Plasmid replication protein [Hungatella hathewayi]
>WP_028509833.1 Rep family protein [Ruminococcus sp. NK3A76]
>WP_033683822.1 Rep family protein [Streptococcus mitis]
>EFO53527.1 replication protein [Streptococcus infantis SK1302]
>WP_049523992.1 Rep family protein [Streptococcus pseudopneumoniae]
>WP_024410839.1 Rep family protein [Streptococcus suis]
>CYX46115.1 replication initiator protein [Streptococcus suis]
>CYW87437.1 replication initiator protein [Streptococcus suis]
>WP_051448806.1 Rep family protein [Viridibacillus arenosi]
>WP_050444210.1 Rep family protein [Enterococcus faecalis]
>WP_010817837.1 MULTISPECIES: Rep family protein [Enterococcus]
>WP_016226904.1 Rep family protein [Lachnospiraceae bacterium 10-1]
>SCH55298.1 Plasmid replication protein [uncultured Collinsella sp.]
>WP_042900192.1 Rep family protein, partial [Streptococcus mitis]
>WP_050492321.1 Rep family protein [Streptococcus mitis]
>KXA58447.1 putative plasmid replication protein [Streptococcus mitis]
>WP_000093566.1 Rep family protein [Streptococcus mitis]
>EEJ43069.1 hypothetical protein HMPREF0555_0290 [Leuconostoc mesenteroides subsp. cremoris
ATCC 19254]
>WP_004900270.1 Rep family protein [Leuconostoc citreum]
>WP_062359070.1 Rep family protein [Weissella sp. DD23]
>WP_008469878.1 Rep family protein [Lactobacillus hominis]
>WP_046324376.1 MULTISPECIES: Rep family protein [Lactobacillus]
>WP_049150683.1 Rep family protein [Lactobacillus gasseri]
>WP_014567781.1 Rep family protein [Lactobacillus johnsonii]
>WP_007125042.1 Rep family protein [Lactobacillus ultunensis]
>WP_060461663.1 Rep family protein [Lactobacillus crispatus]
>WP_013641481.1 Rep family protein [Lactobacillus amylovorus]
>WP_003549058.1 Rep family protein [Lactobacillus acidophilus]
>KRN00682.1 replication protein [Lactobacillus taiwanensis DSM 21401]
>CDA26462.1 putative replication initiator protein [Lactobacillus amylovorus CAG: 719]
>CDI43023.1 Putative replication initiator protein [Lactobacillus helveticus CIRM-BIA 953]
>KRK41125.1 replication protein [Lactobacillus amylovorus DSM 20531]
>WP_012845653.1 Rep family protein [Lactobacillus johnsonii]
>CDI42894.1 Putative replication initiator protein [Lactobacillus helveticus CIRM-BIA 953]
>WP_011254167.1 Rep family protein [Lactobacillus acidophilus]
>WP_056985318.1 Rep family protein [Lactobacillus amylovorus]
>WP_008472153.1 Rep family protein [Lactobacillus gigeriorum]
>WP_013641468.1 Rep family protein [Lactobacillus amylovorus]
>WP_057906729.1 Rep family protein [Ligilactobacillus araffinosus]
>WP_057827851.1 Rep family protein [Ligilactobacillus aviarius]
>WP_057827085.1 Rep family protein [Ligilactobacillus aviarius]
>WP_016356676.1 Rep family protein [Carnobacterium maltaromaticum]
>WP_016622553.1 Rep family protein [Enterococcus faecalis]
>ABP89830.1 putative replication initiator protein [Streptococcus suis 05ZYH33]
>CUR41281.1 Replication protein [Limosilactobacillus reuteri]
>KRN07545.1 hypothetical protein FD15_GL000830 [Liquorilactobacillus sucicola DSM 21376 = JCM
15457]
>WP_003665528.1 Rep family protein [Limosilactobacillus reuteri]
>WP_006499656.1 Rep family protein [Limosilactobacillus mucosae]
>WP_046923918.1 Rep family protein [Ligilactobacillus ruminis]
>AKG47101.1 replication initiation protein (plasmid) [Limosilactobacillus vaginalis]
>WP_046025501.1 Rep family protein [Limosilactobacillus fermentum]
>WP_034540695.1 Rep family protein [Limosilactobacillus mucosae]
>WP_002821392.1 Rep family protein [Oenococcus oeni]
>YP_006939186.1 putative replication initiator protein (plasmid) [Staphylococcus epidermidis]
>WP_000818357.1 Rep family protein [Streptococcus agalactiae]
>WP_000186194.1 Rep family protein [Streptococcus agalactiae]
>CBL40434.1 RNA helicase./Plasmid replication protein [butyrate-producing bacterium SS3/4]
>CRY93789.1 hypothetical protein (plasmid) [uncultured prokaryote]
>WP_021639163.1 Rep family protein [Clostridium sp. KLE 1755]
>WP_007889993.1 hypothetical protein [Roseburia inulinivorans]
>CRY97508.1 hypothetical protein (plasmid) [uncultured prokaryote]
>CDA18875.1 rNA helicase./Plasmid replication protein [Ruminococcus sp. CAG: 488]
>WP_017824301.1 Rep family protein [Brachybacterium muris]
>CEI31812.1 Plasmid replication protein [Propionibacterium freudenreichii]
>WP_044572803.1 hypothetical protein [Arthrobacter alpinus]
>WP_067940518.1 RNA helicase [Actinomyces radicidentis]
>WP_005464724.1 RNA helicase [Saccharomonospora glauca]
>WP_043534193.1 RNA helicase [Actinomyces polynesiensis]
>GAC78794.1 hypothetical protein GM1_004_02390 [Gordonia malaquae NBRC 108250]
>WP_006681830.1 Rep family protein [Schaalia turicensis]
>WP_052038917.1 Rep family protein [Actinomyces sp. S4-C9]
>WP_024390948.1 Rep family protein [Streptococcus suis]
>WP_029176301.1 Rep family protein [Streptococcus suis]
>WP_024393234.1 Rep family protein [Streptococcus suis]
>WP_050238550.1 Rep family protein [Streptococcus pneumoniae]
>WP_061866456.1 Rep family protein [Streptococcus oralis]
>WP_058211405.1 Rep family protein [Lactococcus lactis]
>WP_017368666.1 Rep family protein [Lactococcus garvieae]
>WP_038978316.1 Rep family protein [Paenibacillus polymyxa]
>WP_046467524.1 Rep family protein [Staphylococcus pasteuri]
>WP_061417941.1 Rep family protein [Streptococcus oralis]
>WP_061863770.1 MULTISPECIES: Rep family protein [Streptococcus]
>WP_049535277.1 Rep family protein [Streptococcus pseudopneumoniae]
>WP_049478725.1 MULTISPECIES: Rep family protein [Streptococcus]
>WP_067193806.1 Rep family protein [Streptococcus sp. DD10]
>WP_044771983.1 Rep family protein [Streptococcus suis]
>WP_024408358.1 Rep family protein [Streptococcus suis]
>WP_039694423.1 Rep family protein [Streptococcus gallolyticus]
>WP_033583888.1 MULTISPECIES: Rep family protein [Streptococcus]
>WP_049481849.1 Rep family protein [Streptococcus constellatus]
>WP_029171254.1 Rep family protein [Streptococcus suis]
>WP_053863690.1 Rep family protein [Streptococcus suis]
>WP_014735272.1 Rep family protein [Streptococcus suis]
>WP_018166163.1 Rep family protein [Streptococcus minor]
>WP_024382134.1 Rep family protein [Streptococcus suis]
>WP_024399566.1 Rep family protein [Streptococcus suis]
>WP_024389873.1 Rep family protein [Streptococcus suis]
>AAC34816.1 BFDV nuclease domain
>CAC44748.2 CaCV nuclease domain
>YP_009506307.1 Chimp11 nuclease domain
>YP_009506281.1 Chimp17 nuclease domain
>AF252610 CoCV nuclease domain
>ALO24183.1 DCV nuclease domain
>ASL05580.1 FBYNV nuclease domain
>ABI54257.1 FiCV nuclease domain
>CAC50261.1 GoCV nuclease domain
>ABI54254.1 GuCV nuclease domain
>AST11805.1 MSMV nuclease domain
>YP_009506325.1 NG12 nuclease domain
>YP_009506283.1 NG13 nuclease domain
>YP_009506327.1 NG14 nuclease domain
>AAT72755.1 PCV1 nuclease domain
>NP_937956.1 PCV2 nuclease domain
>YP_009458619.1 PK5006 nuclease domain
>YP_009506323.1 PK5034 nuclease domain
>YP_009506319.1 PK5222 nuclease domain
>YP_009506317.1 PK5510 nuclease domain
>ABA53822.1 RaCV nuclease domain
>WP_050203274.1 RepBm nuclease domain
>DQ1729062 StCV nuclease domain
>ABU48445.1 SwCV nuclease domain
>YP_009506321.1 TN25 nuclease domain
>AEI91450.1 TGMV nuclease domain
>CAD5542681.1 Tral nuclease domain

### REFERENCES

1. Yeh, C. D., Richardson, C. D. & Corn, J. E. Advances in genome editing through control of DNA repair pathways. Nat. Cell Biol. 21, 1468-1478 (2019).
2. van Overbeek, M. et al. DNA Repair Profiling Reveals Nonrandom Outcomes at Cas9-Mediated Breaks. Mol. Cell 63, 633-646 (2016).
3. Kosicki, M., Tomberg, K. & Bradley, A. Repair of double-strand breaks induced by CRISPR-Cas9 leads to large deletions and complex rearrangements. Nat. Biotechnol. 36, 765-771 (2018).
4. Leibowitz, M. L. et al. Chromothripsis as an on-target consequence of CRISPR-Cas9 genome editing. Nat. Genet. 53, 895-905 (2021).
5. Alanis-Lobato, G. et al. Frequent loss of heterozygosity in CRISPR-Cas9-edited early human embryos. Proc. Natl. Acad. Sci. 118, (2021).
6. Enache, O. M. et al. Cas9 activates the p53 pathway and selects for p53-inactivating mutations. Nat. Genet. 52, 662-668 (2020).
7. Morgens, D. W. et al. Genome-scale measurement of off-target activity using Cas9 toxicity in high-throughput screens. Nat. Commun. 8, 15178 (2017).
8. Cilley, C. D. & Williamson, J. R. Analysis of bacteriophage N protein and peptide binding to boxB RNA using polyacrylamide gel coelectrophoresis (PACE). RNA N. Y. N 3, 57-67 (1997).
9. Smiley, A. T. & Gordon, W. R. Engineering sequence-directed protein-RNA covalent linkages for compact RNA-labeling tools. Biophys. J. 122, 216a (2023).
10. Doman, J. L. et al. Phage-assisted evolution and protein engineering yield compact, efficient prime editors. Cell 186, 3983-4002.e26 (2023).
11. Austin, R. J., Xia, T., Ren, J., Takahashi, T. T. & Roberts, R. W. Differential Modes of Recognition in N Peptide-BoxB Complexes. Biochemistry 42, 14957-14967 (2003).
12. CILLEY, C. D. & WILLIAMSON, J. R. Structural mimicry in the phage cφ21 N peptide-boxB RNA complex. RNA 9, 663-676 (2003).
13. Wolfram-Schauerte, M. et al. A viral ADP-ribosyltransferase attaches RNA chains to host proteins. Nature 1-9 (2023) doi:10.1038/s41586-023-06429-2.
14. Peabody, D. S. The RNA binding site of bacteriophage MS2 coat protein. EMBO J. 12, 595-600 (1993).
15. Lim, F. & Peabody, D. S. RNA recognition site of PP7 coat protein. Nucleic Acids Res. 30, 4138-4144 (2002).
16. Gebauer, F., Schwarzl, T., Valcarcel, J. & Hentze, M. W. RNA-binding proteins in human genetic disease. Nat. Rev. Genet. 22, 185-198 (2021).
17. Castello, A. et al. Comprehensive Identification of RNA-Binding Domains in Human Cells. Mol. Cell 63, 696-710 (2016).
18. Spencer, J. M. & Zhang, X. Deep mutational scanning of S. pyogenes Cas9 reveals important functional domains. Sci. Rep. 7, 16836 (2017).
19. Kazlauskas, D., Varsani, A., Koonin, E. V. & Krupovic, M. Multiple origins of prokaryotic and eukaryotic single-stranded DNA viruses from bacterial and archaeal plasmids. Nat. Commun. 10, 3425 (2019).
20. Anzalone, A. V. et al. Search-and-replace genome editing without double-strand breaks or donor DNA. Nature 576, 149-157 (2019).
21. Plaper, T. et al. Coiled-coil heterodimers with increased stability for cellular regulation and sensing SARS-CoV-2 spike protein-mediated cell fusion. Sci. Rep. 11, 9136 (2021).
22. Lainšček, D. et al. Coiled-coil heterodimer-based recruitment of an exonuclease to CRISPR/Cas for enhanced gene editing. Nat. Commun. 13, 3604 (2022).
23. Clement, K. et al. CRISPResso2 provides accurate and rapid genome editing sequence analysis. Nat. Biotechnol. 37, 224-226 (2019).
24. Bae, S., Park, J. & Kim, J.-S. Cas-OFFinder: a fast and versatile algorithm that searches for potential off-target sites of Cas9 RNA-guided endonucleases. Bioinformatics 30, 1473-1475 (2014).
25. Anzalone, A. V., Koblan, L. W. & Liu, D. R. Genome editing with CRISPR-Cas nucleases, base editors, transposases and prime editors. Nat. Biotechnol. 38, 824-844 (2020).
26. Komor, A. C., Kim, Y. B., Packer, M. S., Zuris, J. A. & Liu, D. R. Programmable editing of a target base in genomic DNA without double-stranded DNA cleavage. Nature 533, 420-424 (2016).
27. Koblan, L. W. et al. Improving cytidine and adenine base editors by expression optimization and ancestral reconstruction. Nat. Biotechnol. 36, 843-846 (2018).
28. Gaudelli, N. M. et al. Programmable base editing of A·T to G·C in genomic DNA without DNA cleavage. Nature 551, 464-471 (2017).
29. Richter, M. F. et al. Phage-assisted evolution of an adenine base editor with improved Cas domain compatibility and activity. Nat. Biotechnol. 38, 883-891 (2020).
30. Anzalone, A. V. et al. Search-and-replace genome editing without double-strand breaks or donor DNA. Nature 576, 149-157 (2019).
31. Berdis, A. J. Mechanisms of DNA Polymerases. Chem. Rev. 109, 2862-2879 (2009).
32. Johansson, E. & Dixon, N. Replicative DNA Polymerases. Cold Spring Harb. Perspect. Biol. 5, a012799 (2013).
33. Egli, M. & Manoharan, M. Chemistry, structure and function of approved oligonucleotide therapeutics. Nucleic Acids Res. gkad067 (2023) doi:10.1093/nar/gkad067.
34. Chandler, M. et al. Breaking and joining single-stranded DNA: the HUH endonuclease superfamily. Nat. Rev. Microbiol. 11, 525-538 (2013).
35. Lovendahl, K. N., Hayward, A. N. & Gordon, W. R. Sequence-Directed Covalent Protein-DNA Linkages in a Single Step Using HUH-Tags. J. Am. Chem. Soc. 139, 7030-7035 (2017).
36. Tompkins, K. J. et al. Molecular underpinnings of ssDNA specificity by Rep HUH-endonucleases and implications for HUH-tag multiplexing and engineering. Nucleic Acids Res. 49, 1046-1064 (2021).
37. Aird, E. J., Lovendahl, K. N., St. Martin, A., Harris, R. S. & Gordon, W. R. Increasing Cas9-mediated homology-directed repair efficiency through covalent tethering of DNA repair template. Commun. Biol. 1, 1-6 (2018).
38. Richardson, C. D., Ray, G. J., DeWitt, M. A., Curie, G. L. & Corn, J. E. Enhancing homology-directed genome editing by catalytically active and inactive CRISPR-Cas9 using asymmetric donor DNA. Nat. Biotechnol. 34, 339-344 (2016).
39. Zalatan, J. G. et al. Engineering Complex Synthetic Transcriptional Programs with CRISPR RNA Scaffolds. Cell 160, 339-350 (2015).
40. Nowak, C. M., Lawson, S., Zerez, M. & Bleris, L. Guide RNA engineering for versatile Cas9 functionality. Nucleic Acids Res. 44, 9555-9564 (2016).
41. Li, L. et al. Multiple Diverse Circoviruses Infect Farm Animals and Are Commonly Found in Human and Chimpanzee Feces. J. Virol. 84, 1674-1682 (2010).
42. Barnes, W. M., Zhang, Z. & Kermekchiev, M. B. A Single Amino Acid Change to Taq DNA Polymerase Enables Faster PCR, Reverse Transcription and Strand-Displacement. Front. Bioeng. Biotechnol. 8, 553474 (2021).
43. Arezi, B. et al. Compartmentalized self-replication under fast PCR cycling conditions yields Taq DNA polymerase mutants with increased DNA-binding affinity and blood resistance. Front. Microbiol. 5, 408 (2014).
44. Ignatov, K. B., Bashirova, A. A., Miroshnikov, A. I. & Kramarov, V. M. Mutation S543N in the thumb subdomain of the Taq DNA polymerase large fragment suppresses pausing associated with the template structure. FEBS Lett. 448, 145-148 (1999).
45. Patel, P. H. & Loeb, L. A. DNA polymerase active site is highly mutable: Evolutionary consequences. Proc. Natl. Acad. Sci. 97, 5095-5100 (2000).
46. Yamagami, T., Ishino, S., Kawarabayasi, Y. & Ishino, Y. Mutant Taq DNA polymerases with improved elongation ability as a useful reagent for genetic engineering. Front. Microbiol. 5, (2014).
47. Wang, Y. A novel strategy to engineer DNA polymerases for enhanced processivity and improved performance in vitro. Nucleic Acids Res. 32, 1197-1207 (2004).
48. Paik, I., Bhadra, S. & Ellington, A. D. Charge Engineering Improves the Performance of Bst DNA Polymerase Fusions. ACS Synth. Biol. 11, 1488-1496 (2022).
49. Olszewski, M., Spibida, M., Bilek, M. & Krawczyk, B. Fusion of Taq DNA polymerase with single-stranded DNA binding-like protein of Nanoarchaeum equitans-Expression and characterization. PloS One 12, e0184162 (2017).
50. Villbrandt, B., Sobek, H., Frey, B. & Schomburg, D. Domain exchange: chimeras of Thermus aquaticus DNA polymerase, Escherichia coli DNA polymerase I and Thermotoga neapolitana DNA polymerase. Protein Eng. 13, 645-654 (2000).
51. Durrant, M. G. et al. Systematic discovery of recombinases for efficient integration of large DNA sequences into the human genome. Nat. Biotechnol. (2022) doi:10.1038/s41587-022-01494-w.
52. Chandler, M. et al. Breaking and joining single-stranded DNA: the HUH endonuclease superfamily. Nat. Rev. Microbiol. 11, 525-538 (2013).
53. Lovendahl, K. N., Hayward, A. N. & Gordon, W. R. Sequence-Directed Covalent Protein-DNA Linkages in a Single Step Using HUH-Tags. J. Am. Chem. Soc. 139, 7030-7035 (2017).
54. Zdechlik, A. C., He, Y., Aird, E. J., Gordon, W. R. & Schmidt, D. Programmable Assembly of Adeno-Associated Virus-Antibody Composites for Receptor-Mediated Gene Delivery. Bioconjug. Chem. 31, 1093-1106 (2020).
55. Zhou, K. et al. Toward Precise Manipulation of DNA-Protein Hybrid Nanoarchitectures. Small 15, 1804044 (2019).
56. Ngo, T. A. et al. Protein adaptors assemble functional proteins on DNA scaffolds. Chem. Commun. 55, 12428-12446 (2019).
57. Sagredo, S. et al. Orthogonal Protein Assembly on DNA Nanostructures Using Relaxases. Angew. Chem. Int. Ed. 55, 4348-4352 (2016).
58. Guo, W., Mashimo, Y., Kobatake, E. & Mie, M. Construction of DNA-displaying nanoparticles by enzymatic conjugation of DNA and elastin-like polypeptides using a replication initiation protein. Nanotechnology 31, 255102 (2020).
59. Aird, E. J., Lovendahl, K. N., St. Martin, A., Harris, R. S. & Gordon, W. R. Increasing Cas9-mediated homology-directed repair efficiency through covalent tethering of DNA repair template. Commun. Biol. 1, 54 (2018).
60. Chen, P. J. et al. Enhanced prime editing systems by manipulating cellular determinants of editing outcomes. Cell 184, 5635-5652.e29 (2021).
61. Ferreira da Silva, J. et al. Prime editing efficiency and fidelity are enhanced in the absence of mismatch repair. Nat. Commun. 13, 760 (2022).
62. Kleinstiver, B. P. et al. Engineered CRISPR-Cas9 nucleases with altered PAM specificities. Nature 523, 481-485 (2015).
63. Rohland, N. & Reich, D. Cost-effective, high-throughput DNA sequencing libraries for multiplexed target capture. Genome Res. 22, 939-946 (2012).
64. Kleinstiver, B. P. et al. Engineered CRISPR-Cas12a variants with increased activities and improved targeting ranges for gene, epigenetic and base editing. Nat. Biotechnol. 37, 276-282 (2019).
65. Clement, K. et al. CRISPResso2 provides accurate and rapid genome editing sequence analysis. Nat. Biotechnol. 37, 224-226 (2019).
66. Iseli, C., Ambrosini, G., Bucher, P. & Jongeneel, C. V. Indexing Strategies for Rapid Searches of Short Words in Genome Sequences. PLOS ONE 2, e579 (2007).
67. Tsai, S. Q. et al. GUIDE-seq enables genome-wide profiling of off-target cleavage by CRISPR-Cas nucleases. Nat. Biotechnol. 33, 187-197 (2015).
68. Tabor, S. & Richardson, C. C. Selective inactivation of the exonuclease activity of bacteriophage T7 DNA polymerase by in vitro mutagenesis. J. Biol. Chem. 264, 6447-6458 (1989).
69. Klenow, H. & Overgaard-Hansen, K. Proteolytic cleavage of DNA polymerase from Escherichia Coli B into an exonuclease unit and a polymerase unit. FEBS Lett. 6, 25-27 (1970).
70. Komor, A. C., Kim, Y. B., Packer, M. S., Zuris, J. A. & Liu, D. R. Programmable editing of a target base in genomic DNA without double-stranded DNA cleavage. Nature 533, 420-424 (2016).
71. Lawyer, F. C. et al. High-level expression, purification, and enzymatic characterization of full-length Thermus aquaticus DNA polymerase and a truncated form deficient in 5′ to 3′ exonuclease activity. Genome Res. 2, 275-287 (1993).
72. Blanco, L. et al. Highly efficient DNA synthesis by the phage phi 29 DNA polymerase.

Symmetrical mode of DNA replication. J. Biol. Chem. 264, 8935-8940 (1989).
73. Esteban, J. A., Soengas, M. S., Salas, M. & Blanco, L. 3′->5′ exonuclease active site of phi 29 DNA polymerase. Evidence favoring a metal ion-assisted reaction mechanism. J. Biol. Chem. 269, 31946-31954 (1994).
74. Thyme, S. B., Akhmetova, L., Montague, T. G., Valen, E. & Schier, A. F. Internal guide RNA interactions interfere with Cas9-mediated cleavage. Nat. Commun. 7, 11750 (2016).
75. Ponnienselvan, K. et al. Reducing the inherent auto-inhibitory interaction within the pegRNA enhances prime editing efficiency. Nucleic Acids Res. 51, 6966-6980 (2023).
76. Chen, P. J. et al. Enhanced prime editing systems by manipulating cellular determinants of editing outcomes. Cell 184, 5635-5652.e29 (2021).
77. Ferreira da Silva, J. et al. Prime editing efficiency and fidelity are enhanced in the absence of mismatch repair. Nat. Commun. 13, 760 (2022).
78. Petri, K. et al. CRISPR prime editing with ribonucleoprotein complexes in zebrafish and primary human cells. Nat. Biotechnol. 40, 189-193 (2022).
79. Grunewald, J. et al. Engineered CRISPR prime editors with compact, untethered reverse transcriptases. Nat. Biotechnol. 41, 337-343 (2023).
80. Li, X. et al. Highly efficient prime editing by introducing same-sense mutations in pegRNA or stabilizing its structure. Nat. Commun. 13, 1669 (2022).
81. Liu, B. et al. A split prime editor with untethered reverse transcriptase and circular RNA template. Nat. Biotechnol. 40, 1388-1393 (2022).
82. Kim, D. Y., Moon, S. B., Ko, J.-H., Kim, Y.-S. & Kim, D. Unbiased investigation of specificities of prime editing systems in human cells. Nucleic Acids Res. 48, 10576-10589 (2020).
83. Yu, Z. et al. PEAC-seq adopts Prime Editor to detect CRISPR off-target and DNA translocation. Nat. Commun. 13, 7545 (2022).
84. Liang, S.-Q. et al. Genome-wide detection of CRISPR editing in vivo using GUIDE-tag. Nat. Commun. 13, 437 (2022).
85. Liang, S.-Q. et al. Genome-wide profiling of prime editor off-target sites in vitro and in vivo using P E-tag. Nat. Methods 20, 898-907 (2023).
86. Kamtekar, S. et al. Insights into strand displacement and processivity from the crystal structure of the protein-primed DNA polymerase of bacteriophage phi29. Mol. Cell 16, 609-618 (2004).
87. Rodriguez, I. et al. A specific subdomain in phi29 DNA polymerase confers both processivity and strand-displacement capacity. Proc. Natl. Acad. Sci. U.S.A 102, 6407-6412 (2005).
88. de Vega, M., Lázaro, J. M., Mencía, M., Blanco, L. & Salas, M. Improvement of φ29 DNA polymerase amplification performance by fusion of DNA binding motifs. Proc. Natl. Acad. Sci. U.S.A 107, 16506-16511 (2010).
89. Povilaitis, T., Alzbutas, G., Sukackaite, R., Siurkus, J. & Skirgaila, R. In vitro evolution of phi29 DNA polymerase using isothermal compartmentalized self replication technique. Protein Eng. Des. Sel. 29, 617-628 (2016).
90. Ong, J., Tanner, N., Zhang, Y., Bei, Y. & Potapov, V. Variant DNA Polymerases Having Improved Properties and Method for Improved Isothermal Amplification of a Target DNA. (2021).
91. Derbyshire, V. et al. Genetic and crystallographic studies of the 3′,5′-exonucleolytic site of DNA polymerase I. Science 240, 199-201 (1988).
92. Polesky, A. H., Dahlberg, M. E., Benkovic, S. J., Grindley, N. D. & Joyce, C. M. Side chains involved in catalysis of the polymerase reaction of DNA polymerase I from Escherichia coli. J. Biol. Chem. 267, 8417-8428 (1992).
93. Khil, P. P., Smagulova, F., Brick, K. M., Camerini-Otero, R. D. & Petukhova, G. V. Sensitive mapping of recombination hotspots using sequencing-based detection of ssDNA. Genome Res. 22, 957-965 (2012).
94. Nesta, A. V., Tafur, D. & Beck, C. R. Hotspots of Human Mutation. Trends Genet. 37, 717-729 (2021).
95. Blitzblau, H. G., Bell, G. W., Rodriguez, J., Bell, S. P. & Hochwagen, A. Mapping of Meiotic Single-Stranded DNA Reveals Double-Strand-Break Hotspots near Centromeres and Telomeres. Curr. Biol. 17, 2003-2012 (2007).
96. Merrick J Z, et al., ACS Synth. Biol. 2018. Serine Integrases: Advancing Synthetic Biology; Rutherford K, et al., Curr Opin Struct Biol. 2014. The ins and outs of serine integrase site-specific recombination

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A click editor fusion protein comprising a DNA binding domain, a clkNA tethering domain,
and an effector domain, with optional linkers therebetween, optionally wherein:
(i) the DNA binding domain, clkNA tethering domain, and effector domain are fused in any order; or
(ii) the clkNA tethering domain and/or the effector domain is inlaid internally into the DNA binding domain,
wherein the DNA binding domain comprises an RNA-programmable DNA nickase or nuclease.

2. (canceled)

3. The click editor fusion proteins of claim 1, wherein the clkNA tethering domain is:
(i) a sequence-specific covalent or non-covalent ssDNA binding domain optionally an HUH endonuclease or telomere binding protein;
(ii) a domain that covalently or noncovalently binds a chemical moiety on the clkNA, optionally Avidin, SNAP-tag, CLIP-tag, HALO-tag; or
(iii) an RNA-binding domain, optionally an engineered RNA-binding HUH endonuclease or Telomere binding protein, or a Phage coat protein (CP), optionally MCP, PCP, N21p, N22p, BoxB, or Com.

4. The click editor fusion proteins of claim 1, wherein the effector domain is a DNA-dependent DNA polymerase or ligase.

5.-6. (canceled)

7. A click nucleic acid (clkNA) template, optionally 15-500 nt long, comprising (i) a localization moiety, (ii) a polymerization template (PT) comprising at least one desired edit or an attachment duplex region (ADR) comprising at least one desired edit, and (iii) a flap binding region (FBR), wherein the clkNA template comprises RNA, DNA (clkDNA) or both RNA and DNA, wherein:
the localization moiety is an HUH endonuclease recognition sequence (RNA or DNA), a Telomere Binding Protein recognition sequence, biotin, benzylguanine derivative, benzylcytosine derivative, a chloroalkane, or an RNA sequence that is bound by an RNA binding protein, optionally a phage coat protein (CP) or phage antitermination signal, optionally MS2, PP7, BoxB, or Com;
the polymerization template (PT) comprises a portion that binds to the target genome, optionally at least 3, 4, 5, 6, 7, 8, 9, or 10 nt long, and up to 500 nt long, and a portion that includes at least one desired edit that is at least 1 nt long:
the ADR comprises a dsDNA portion that comprises a homology to the target genome, optionally at least 3, 4, 5, 6, 7, 8, 9, or 10 nt long, and up to 500 nt long with at least one desired edit that is at least 1 nt long; and/or
the flap binding region is complementary to a genomic flap released by a nickase, optionally wherein the flap binding region is 5-50 nt in length, and is immediately 3′ of the PT or ADR.

8.-11. (canceled)

12. A composition comprising:
(i) the click editor fusion protein of claim 1;
(ii) a clkNA template comprising (i) a localization moiety, (ii) a polymerization template (PT) comprising at least one desired edit or an attachment duplex region (ADR) comprising at least one desired edit, and (iii) a flap binding region (FBR), wherein the clkNA template comprises RNA, DNA (clkDNA) or both RNA and DNA, and further wherein the localization moiety of the clkNA template binds to the clkNA tethering domain of the click editor fusion protein; and
(iii) at least one guide RNA that directs the RNA-programmable DNA nickase to a target DNA sequence, and optionally a second guide RNA that directs the RNA-programmable DNA nickase to a target DNA sequence on the opposite strand.

13. A click editor composition comprising a DNA binding domain, a clkNA tethering domain, and an effector domain, wherein the clkNA tethering domain and the DNA binding domain are in a single fusion protein, and the effector domain is separate, wherein;
the DNA binding domain is an RNA-programmable nickase or nuclease;
the clkNA tethering domain is an HUH endonuclease or a Telomere Binding Protein:
the effector domain is a DNA-dependent DNA polymerase or DNA ligase, and
optionally wherein the fusion protein and the effector domain can form a non-covalent complex by interaction of protein recruitment domains on each of the fusion protein and the effector domain, optionally wherein the protein recruitment domains are interacting leucine zipper domains.

14.-24. (canceled)

25. The composition of claim 12, further comprising a recombinase fused to the complex or fusion protein comprising the DBD, recruited to the DBD by a protein recruitment domain, or expressed separately in trans.

26. A method of altering a target DNA sequence, optionally a genomic sequence, the method comprising contacting the DNA sequence with:
(i) a DNA binding domain (comprising an RNA-programmable DNA nickase or nuclease, a clkNA tethering domain, and an effector domain, optionally in one or more fusion proteins;
(ii) the clkNA template of claim 7, wherein the localization moiety of the clkNA template binds to the clkNA tethering domain of the click editor fusion protein; and
(iii) a guide RNA that directs the RNA-programmable DNA nickase to the target DNA sequence,
wherein:
the RNA-programmable DNA nickase nicks the non-target strand at the target site;
the FBR on the clkNA template anneals to the non-target DNA strand; and
an extended 3′ DNA flap is generated by the effector domain,
wherein the 3′ DNA flap is incorporated into the target DNA, leading to altering the target DNA.

27. A method of altering a target DNA sequence, optionally by deletion, replacement, or duplication of the target DNA sequence, the method comprising contacting the DNA sequence with:
(i) a DNA binding domain (comprising an RNA-programmable nickase or nuclease, a clkNA tethering domain, and an effector domain, optionally in one or more fusion proteins;
(ii) the clkNA template of claim 7, wherein the localization moiety of the clkNA template binds to the clkNA tethering domain of the click editor fusion protein; and
(iii) a pair of sgRNAs, each targeting opposite DNA strands, to generate two extended 3′ flaps that are either complementary to upstream sequence or to each other.

28. A method of altering a target DNA sequence, optionally a genomic sequence, the method comprising contacting the DNA sequence with:
(i) a DNA binding domain (comprising an RNA-programmable DNA nickase or nuclease linked to a clkNA tethering domain with optional linkers therebetween,
(ii) the clkNA template of claim 7, wherein the localization moiety of the clkNA template binds to the clkNA tethering domain;
(iii) an effector domain linked to an RNA binding protein, optionally MCP, PCP, or Com RNA binding protein; and
(iv) one or a pair of sgRNAs, each targeting opposite DNA strands, to generate two extended 3′ flaps that are either complementary to upstream sequence or to each other, wherein one or both of the sgRNAs comprises a MS2, PP7, or com sequence.

29. A method of altering a target DNA sequence, optionally a genomic sequence, the method comprising contacting the DNA sequence with:
(i) a DNA binding domain (comprising an RNA-programmable DNA nickase or nuclease, a clkNA tethering domain, and an effector domain, optionally in one or more fusion proteins;
(ii) the clkNA template of claim 7, comprising a localization moiety, a polymerization template (PT) comprising at least one desired edit or an attachment duplex region (ADR) comprising at least one desired edit, a first flap binding region (PBS1), and the reverse complement of a second PBS (rcPBS 2), wherein the localization moiety of the clkNA template binds to the clkNA tethering domain of the click editor fusion protein; and
(iii) a pair of guide RNAs that direct the RNA-programmable DNA nickase to first and second sites on the target DNA sequence,
wherein:
the RNA-programmable DNA nickase creates first and second nicks on the non-target strand at the target site;
the PBS1 on the clkNA template anneals to the non-target DNA strand at the first nick site;
an extended 3′ DNA flap comprising a sequence complementary to PBS2 is generated by the effector domain,
the extended 3′ DNA flap comprising PBS2 anneals to the 3′ flap at the second nick site;
the effector domain carries out second strand synthesis; and
the newly synthesized DNA is incorporated into the target DNA, leading to altering the target DNA.

30. The method of claim 26, wherein the edit comprises insertion of an attP or attB sequence, and the method further comprises contacting the DNA with a donor template comprising attR and attL sequences, and a serine recombinase, optionally BxBl or Pa01, optionally fused to the click editor fusion protein.

31. The clkNA template of claim 7, wherein the edit comprises insertion of an attP or attB sequence.

32. The clkNA template of claim 7, wherein the clkNA template is all DNA (clkNA) or partly DNA and partly RNA, wherein the HUH endonuclease sequence is DNA and the rest is RNA; the HUH endonuclease sequence is DNA, all or a portion of the FBR is RNA and the PT is DNA; or the HUH endonuclease sequence is DNA, and all or a portion of the FBR is RNA and all or a portion of the PT is RNA; or any of the above where the HUH endonuclease sequence is RNA instead of DNA.

33. The clkNA template of claim 7 wherein the clkNA template comprises one or more chemical modifications, optionally a modified sugar moiety and/or a modified internucleoside linkage.
